SEQUENCE LISTING (182 sequences)

SEQ ID NO: 1 - protein, 22 aa, Saccharomyces cerevisiae
MFSLKALLPLALLLVSANQVAA

SEQ ID NO: 2 - protein, 54 aa, Saccharomyces cerevisiae
KVHKAKIYKHELSDEMKEVTFEQHLAHLGQKYLTQFEKANPEVVFSREHPFFTE

SEQ ID NO: 3 - protein, 18 aa, Homo sapiens
MVRMVPVLLSLLLLLGPA

SEQ ID NO: 4 - protein, 19 aa, Kluyveromyces marxianus
MLFSNTLLIAAASALLAEA

SEQ ID NO: 5 - protein, 19 aa, Saccharomycopsis fibuligera
MKFGVLFSVFAAIVSALPA

SEQ ID NO: 6 - protein, 6 aa, Saccharomyces cerevisiae
GRRAKL

SEQ ID NO: 7-8 - skipped (2 entries)

SEQ ID NO: 9 - protein, 9 aa, Saccharomyces cerevisiae
RRMLSSKQL

SEQ ID NO: 10 - protein, 709 aa, synthetic construct
  REGION 1..709: note = Arthrobacter
MTEVVDRASSPASPGSTTAAADGAKVAVEPRVDVAALGEQLLGRWADIRLHARDLAGREVVQKVEGLTHTEHRSRVFGQLKYLVDNNAVHRAFPSRLGGSDDHGGNIAGFEELVTADPSLQIKAGVQWGLFGSAVMHLGTREHHDKWLPGIMSLEIPGCFAMTETGHGSDVASIATTATYDEETQEFVIDTPFRAAWKDYIGNAANDGLAAVVFAQLITRKVNHGVHAFYVDLRDPATGDFLPGIGGEDDGIKGGLNGIDNGRLHFTNVRIPRTNLLNRYGDVAVDGTYSSTIESPGRRFFTMLGTLVQGRVSLDGAAVAASKVALQSAIHYAAERRQFNATSPTEEEVLLDYQRHQRRLFTRLATTYAASFAHEQLLQKFDDVFSGAHDTDADRQDLETLAAALKPLSTWHALDTLQECREACGGAGFLIENRFASLRADLDVYVTFEGDNTVLLQLVAKRLLADYAKEFRGANFGVLARYVVDQAAGVALHRTGLRQVAQFVADSGSVQKSALALRDEEGQRTLLTDRVQSMVAEVGAALKGAGKLPQHQAAALFNQHQNELIEAAQAHAELLQWEAFTEALAKVDDAGTKEVLTRLRDLFGLSLIEKHLSWYLMNGRLSMQRGRTVGTYINRLLVKIRPHALDLVDAFGYGAEHLRAAIATGAEATRQDEARTYFRQQRASGSAPADEKTLLAIKAGKSRGRRAKL

SEQ ID NO: 11 - protein, 700 aa, Yarrowia lipolytica
MNPNNTGTIEINGKEYNTFTEPPVAMAQERAKTSFPVREMTYFLDGGEKNTLKNEQIMEEIERDPLFNNDNYYDLNKEQIRELTMERVAKLSLFVRDQPEDDIKKRFALIGIADMGTYTRLGVHYGLFFGAVRGTGTAEQFGHWISKGAGDLRKFYGCFSMTELGHGSNLAGLETTAIYDEETDEFIINTPHIAATKWWIGGAAHTATHTVVFARLIVKGKDYGVKTFVVQLRNINDHSLKVGISIGDIGKKMGRDGIDNGWIQFTNVRIPRQNLLMKYTKVDREGNVTQPPLAQLTYGSLITGRVSMASDSHQVGKRFITIALRYACIRRQFSTTPGQPETKIIDYPYHQRRLLPLLAYVYALKMTADEVGALFSRTMLKMDDLKPDDKAGLNEVVSDVKELFSVSAGLKAFSTWACADVIDKTRQACGGHGYSGYNGFGQAYADWVVQCTWEGDNNILTLSAGRALIQSAVALRKGEPVGNAVSYLKRYKDLANAKLNGRSLTDPKVLVEAWEVAAGNIINRATDQYEKLIGEGLNADQAFEVLSQQRFQAAKVHTRRHLIAAFFSRIDTEAGEAIKQPLLNLALLFALWSIEEDSGLFLREGFLEPKDIDTVTELVNKYCTTVREEVIGYTDAFNLSDYFINAPIGCYDGDAYRHYFQKVNEQNPARDPRPPYYASTLKPFLFREEEDDDICELDEE

SEQ ID NO: 12 - protein, 661 aa, Rattus norvegicus
MNPDLRKERASATFNPELITHILDGSPENTRRRREIENLILNDPDFQHEDYNFLTRSQRYEVAVKKSATMVKKMREYGISDPEEIMWFKKLYLANFVEPVGLNYSMFIPTLLNQGTTAQQEKWMRPSQELQIIGTYAQTEMGHGTHLRGLETTATYDPKTQEFILNSPTVTSIKWWPGGLGKTSNHAIVLAQLITQGECYGLHAFVVPIREIGTHKPLPGITVGDIGPKFGYEEMDNGYLKMDNYRIPRENMLMKYAQVKPDGTYVKPLSNKLTYGTMVFVRSFLVGNAAQSLSKACTIAIRYSAVRRQSEIKQSEPEPQILDFQTQQYKLFPLLATAYAFHFVGRYMKETYLRINESIGQGDLSELPELHALTAGLKAFTTWTANAGIEECRMACGGHGYSHSSGIPNIYVTFTPACTFEGENTVMMLQTARFLMKIYDQVRSGKLVGGMVSYLNDLPSQRIQPQQVAVWPTMVDINSLEGLTEAYKLRAARLVEIAAKNLQTHVSHRKSKEVAWNLTSVDLVRASEAHCHYVVVKVFSDKLPKIQDKAVQAVLRNLCLLYSLYGISQKGGDFLEGSIITGAQLSQVNARILELLTLIRPNAVALVDAFDFKDMTLGSVLGRYDGNVYENLFEWAKKSPLNKTEVHESYHKHLKPLQSKL

SEQ ID NO: 13 - protein, 661 aa, Rattus norvegicus
MNPDLRKERASATFNPELITHILDGSPENTRRRREIENLILNDPDFQHEDYNFLTRSQRYEVAVKKSATMVKKMREYGISDPEEIMWFKNSVHRGHPEPLDLHLGMFLPTLLHQATAEQQERFFMPAWNLEITGTYAQTEMGHGTHLRGLETTATYDPKTQEFILNSPTVTSIKWWPGGLGKTSNHAIVLAQLITQGECYGLHAFVVPIREIGTHKPLPGITVGDIGPKFGYEEMDNGYLKMDNYRIPRENMLMKYAQVKPDGTYVKPLSNKLTYGTMVFVRSFLVGNAAQSLSKACTIAIRYSAVRRQSEIKQSEPEPQILDFQTQQYKLFPLLATAYAFHFVGRYMKETYLRINESIGQGDLSELPELHALTAGLKAFTTWTANAGIEECRMACGGHGYSHSSGIPNIYVTFTPACTFEGENTVMMLQTARFLMKIYDQVRSGKLVGGMVSYLNDLPSQRIQPQQVAVWPTMVDINSLEGLTEAYKLRAARLVEIAAKNLQTHVSHRKSKEVAWNLTSVDLVRASEAHCHYVVVKVFSDKLPKIQDKAVQAVLRNLCLLYSLYGISQKGGDFLEGSIITGAQLSQVNARILELLTLIRPNAVALVDAFDFKDMTLGSVLGRYDGNVYENLFEWAKKSPLNKTEVHESYHKHLKPLQSKL

SEQ ID NO: 14 - protein, 664 aa, Glycine max
MEDGVDHLAFERNKAQFDVEDMKIIWAGSRQDFELSDRISRLVASDPAFRKDDRTRLIGRLFKNTLRKAAYAWKRINELRLNEQEAYKLRSFVDQPAFTDLHWGMFVPAIQGQGTDEQQQKWLPLAYGMQIIGCYAQTELGHGSNVQGLETTATFDPKTDEFVIHSPTLTSSKWWPGGLGKISTHAVAYARLIIGGEDHGVHGFIVQLRSLDDHLPLPGITIGDIGMKFGNAAYNTMDNGVLRFDHVRIPRNQMLMRVSQVTREGRYVSSNVPRQLVYGTMVNVRQKIVADASVALSRAVCIATRYSAVRRQFGSHNGGLETQVIDYKTQQARLFPLLASAYAFRFVGGWLKWLYMDVTERLQANDFSTLPEAHACTAGLKSLTTTATADGIEECRKLCGGHGYLCSSGLPELFAVYVPACTYEGDNVVLLLQVARHLMKTVSQLGSGNKPVGTTAYMARVEQLMQYHSDVEKAEDWLKPNVVLEAFEARASRMSVACAQNLSKFANPEEGFQELAADLVDAAVAHCQLIVVSKFIEKLQQDIPGKGVKKQLEVLCSIYALFLLHKHLGDFLSTGCINPKQGSLASEQLRNLYSQVRPNAIALVDAFNYTDHYLGSILGRYDGNVYPKMNEEAWKDPLNDSVVPDGFKEYIQPMLKQQLRNARL

SEQ ID NO: 15 - protein, 665 aa, Glycine max
MEGMVDHLAFERNNSQFDVDEMKIVWAGSRHAFEVSDKMARLVASDPAFRKDDRVVLDRKALFKNTLRKAAYAWKRIIELRLSEEEAAMLRSFVDQPAFTDLHWGMFVPAIKGQGTEEQQKKWLPLAHKMQIIGCYAQTELGHGSNVQGLETTATFDPRTDEFVIHSPTLTSSKWWPGGLGKVSTHAVVYARLITDGQDHGVHGFIVQLRSLDDHLPLPGITVGDIGMKFGNGAYNSMDNGMLRFDHVRIPRNQMLMRVSQVTREGKYVQSSVPRQLVYGTMVYVRQTIVSDASVALSRAVCIATRYSAVRRQFGSKEGGLETQVIDYKTQQARLFPLLASAYAFRFVGEWLKWLYMDVMKRLQASDFSTLPEAHACTAGLKSLTTSATADGIEECRKLCGGHGYLCSSGLPELFAVYIPTCTYEGDNTVLLLQVARHLIKTISQLGSRNKPVGTTSYIGRVEQLMQYRSDVQKVEDWLKPNAVLGAFEARAAKKVVACAQNLSKFTNPEEGFQELSVDLVEAAVAHCQLIVVSKFIEKLQQDIPGKGVKQQLELLCSIYALFLLHKHLGDFLATGCITPKQGSLANELLRSLYSQVRPNAIALVDAFNYTDHYLGSVLGRYDGDVYPKLYEEAWKDPLNDSVVPDGFQEYIRPMLKQQLRNARL

SEQ ID NO: 16 - protein, 684 aa, Beuvaria bassiana
MSFPDNLKPKEPSGSSLLEKERRQSPVDVDALGKHIFAGTSFLERQARVLRAIEQEPLFDKSRQQQLSRVERVKLGLARGKLMRRLQDRHGWDMDDYHMAAYLVGEQSPYRLHVGMFRTTVEEQSSDAQRAYWMPRVNGWEVSGAYSQTELGHGSNVRGVELEARWDPAAREFVVHSPTLTAAKWWNGSLGRTANHAILMAQLMVPDPKREGQYISHGPQAFIAQIRDLKTNLPLEGVVIGDIGVKIGFTSMDNGYMLFNQFRIPHSALLSRYVQLDPETGVFSKSPNPALAYGTMTSIRTMLVEEAGTHLARAVTIAIRYTAIRQQFRDKDSQDPSSAELQVLDYPTVQVRLFPLLAAAFALQYTGKVMRQDYAKTRGEVEKGNLEGLAVMHSNSSGLKSLSTEITNAGIETCRRAMGGHGYGSGSGLVEMQKDYQAKPILEGDNWMITQQTSSFLIKRMTAAAKTRNEPPKDQIDAQLKTFLHQKDKGRTFDILNSDSDIEESFKWRAASMTYDAYEARVIKKKRHNDLLIQFHKLSHAHSQSIMVSSFLTTLTSSNDLAHETKEIVFDLYRLFAYTTIQAESYEFLRCGAASSKDLDALPERIQALLTRIRPHAVKLVDAWKIPDYLLDSALGRYDGNVYEDLFNRAHRLNPLNDIVFNPDYKDDEIVKGSGERKPLSPKL

SEQ ID NO: 17 - protein, 696 aa, Aspergillus nidulans
MPNPPPAWVQALKPASPQGTELLTQERAQSNIDVDTLGDLLHTKEALKKQDEILSVLKSEKVFDKSRNHVLGRTEKIQLALARGKRLQQLKKAHNWSDEDVHVANDLVSEPTPYGLHASMFLVTLREQGTPEQHKLFYERARNYEIIGCYAQTELGHGSNVRGLETTATWDPSDQTFIIHSPTLTASKWWIGSLGRTANHAVVMAQLYIGGKNYGPHPFVVQIRDMETHQPLENVYVGDIGPKFGYNTMDNGFLLFNKLKIPHVNMLARFAQVDKATNKYIRPASPSLMYGTMTWVRSNIVLQAGGVLARGVTIAVRYCAVRRQFQDRDAKANAEENQVLNYKMVQIRLLPLLAAMYALHFTGRGMMRLYEENQERMKAAAQADQEKRGAGPEQLRAGSDLLADLHATSCGLKALASTTAGEGLEVCRRACGGHGYSNYSGIGPWYADYLPTLTWEGDNYMLTQQVARYLLKSARAVLAGKGTANDTSRILQAYLARRDKGASFDILGNDADIVAAFAWRTAHLTFETLKYRDVEKRSWNSLLINFWRLSTALSQYLVVKNFYEAVNSPEIRSSLDKDTASTLRSLFRLHALHTLDREASEFFSSAAVTVRQIGLTQTSEVPKLLDEIRPHAVRLVDSWKIPDWQLDSALGRSDGDVYPDLFKRASMQNPVNDLVFDPYPWNENVLKNAGEIKSKL

SEQ ID NO: 18 - protein, 646 aa, Arabidopsis thaliana
MESRREKNPMTEEESDGLIAARRIQRLSLHLSPSLTPSPSLPLVQTETCSARSKKLDVNGEALSLYMRGKHIDIQEKIFDFFNSRPDLQTPIEISKDDHRELCMNQLIGLVREAGVRPFRYVADDPEKYFAIMEAVGSVDMSLGIKMGVQYSLWGGSVINLGTKKHRDKYFDGIDNLDYTGCFAMTELHHGSNVQGLQTTATFDPLKDEFVIDTPNDGAIKWWIGNAAVHGKFATVFARLILPTHDSKGVSDMGVHAFIVPIRDMKTHQTLPGVEIQDCGHKVGLNGVDNGALRFRSVRIPRDNLLNRFGDVSRDGTYTSSLPTINKRFGATLGELVGGRVGLAYASVGVLKISATIAIRYSLLRQQFGPPKQPEVSILDYQSQQHKLMPMLASTYAYHFATVYLVEKYSEMKKTHDEQLVADVHALSAGLKSYVTSYTAKALSVCREACGGHGYAAVNRFGSLRNDHDIFQTFEGDNTVLLQQVAADLLKRYKEKFQGGTLTVTWSYLRESMNTYLSQPNPVTARWEGEDHLRDPKFQLDAFRYRTSRLLQNVAARLQKHSKTLGGFGAWNRCLNHLLTLAESHIETVILAKFIEAVKNCPDPSAKAALKLACDLYALDRIWKDIGTYRNVDYVAPNKAKVCFLV

SEQ ID NO: 19 - protein, 692 aa, Arabidopsis thaliana
MESRREKNPMTEEESDGLIAARRIQRLSLHLSPSLTPSPSLPLVQTETCSARSKKLDVNGEALSLYMRGKHIDIQEKIFDFFNSRPDLQTPIEISKDDHRELCMNQLIGLVREAGVRPFRYVADDPEKYFAIMEAVGSVDMSLGIKMGVQYSLWGGSVINLGTKKHRDKYFDGIDNLDYTGCFAMTELHHGSNVQGLQTTATFDPLKDEFVIDTPNDGAIKWWIGNAAVHGKFATVFARLILPTHDSKGVSDMGVHAFIVPIRDMKTHQTLPGVEIQDCGHKVGLNGVDNGALRFRSVRIPRDNLLNRFGDVSRDGTYTSSLPTINKRFGATLGELVGGRVGLAYASVGVLKISATIAIRYSLLRQQFGPPKQPEVSILDYQSQQHKLMPMLASTYAYHFATVYLVEKYSEMKKTHDEQLVADVHALSAGLKSYVTSYTAKALSVCREACGGHGYAAVNRFGSLRNDHDIFQTFEGDNTVLLQQVAADLLKRYKEKFQGGTLTVTWSYLRESMNTYLSQPNPVTARWEGEDHLRDPKFQLDAFRYRTSRLLQNVAARLQKHSKTLGGFGAWNRCLNHLLTLAESHIETVILAKFIEAVKNCPDPSAKAALKLACDLYALDRIWKDIGTYRNVDYVAPNKAKAIHKLTEYLSFQVRNVAKELVDAFELPDHVTRAPIAMQSDAYSQYTQVVGF

SEQ ID NO: 20 - protein, 711 aa, Candida viswanathii
  VARIANT 711: note = Xaa = Any Amino Acid
  VARIANT 711: note = Xaa = Any Amino Acid
MTFTKKNVSVSQGPDPRTSIQTERANSKFDPVTMNYFLEGSKERSELMKSLAQQIERDPILFTDGSYYDLTKDQQRELTVLKINRLSRYREGDSVDTFNKRLSIMGVVDPQVATRIGVNLGLFLSCISGNGTAEQFKYWAIDKGTHNIQGLYGCFGMTELGHGSNVAGVETTATFDKETDEFVINTPHIGATKWWIGGAAHSATHCSVYARLVVDGKDYGVKTFVVPLRDSNHDLMPGVTVGDIGAKMGRDGIDNGWIQFSNVRIPRFFMLQKFCKVSAEGEVVLPPLEQLSYSALLGGRVMMVLDSYRMLARVSTIALRYAIGRRQFKGDNVDQNDPNALETQLIDYPLHQKRLFPYLAAAYVVSTGALKVEHTIQSTLATLDAAVENNDTTAIFKSIDDMKSLFIDSGSLKATTTWLAAEAIDQCRQACGGHGYSSYNGFAKAFNDWVVQCTWEGDNNVLSLSVGKPIIKQIIGIEDNGKTVRGSTAFLNQVKDFTGSNASKVVLNNTSDLNDINKVIKSIEVAIIRLAHEAAISVRKESLDFAGAELVQISKLKAHHYLLTEFVKRVGEFEHKELVPFLNTIGRLYSATVVLDKFAGVFLTFNVASPQAITDLASTQIPKLCAEVRPNVVAYTDSFQQSDMVINSAIGKYDGDVYENYFDLVKQLNPPKNTKAPYTAALEGMLNRPSLEARERYEKSDETAAILSKPX

SEQ ID NO: 21 - protein, 662 aa, Candida viswanathii
MPTELQKERELTKFNPKELNYFLEGSQERSEIISNMVEQMQKDPILKVDASYYNLTKDQQREVTAKKIARLSRYFEHEYPDQQAQRLSILGVFDPQVFTRIGVNLGLFVSCVRGNGTNSQFFYWTINKGIDKLRGIYGCFGMTELAHGSNVQGIETTATFDEDTDEFVINTPHIGATKWWIGGAAHSATHCSVYARLKVKGKDYGVKTFVVPLRDSNHDLEPGVTVGDIGAKMGRDGIDNGWIQFSNVRIPRFFMLQKYCKVSRSGEVTMPPSEQLSYSALIGGRVTMMMDSYRMTSRFITIALRYAIHRRQFKKKDTDTIETKLIDYPLHQKRLFPFLAAAYLFSQGALYLEQTMNATNDKLDEAVSAGEKEAIDAAIVESKKLFVASGCLKSTCTWLTAEAIDEARQACGGHGYSSYNGFGKAYSDWVVQCTWEGDNNILAMNVAKPMVRDLLKEPEQKGLVLSSVADLDDPAKLVKAFDHALSGLARDIGAVAEDKGFDITGPSLVLVSKLNAHRFLIDGFFKRITPEWSEVLRPLGFLYADWILTNFGATFLQYGIITPDVSRKISSEHFPALCAKVRPNVVGLTDGFNLTDMMTNAAIGRYDGNVYEHYFETVKALNPPENTKAPYSKALEDMLNRPDLEVRERGEKSEEAAEILSS

SEQ ID NO: 22 - protein, 689 aa, Yarrowia lipolytica
MTTNTFTDPPVEMAKERGKTQFTVRDVTNFLNGGEEETQIVEKIMSSIERDPVLSVTADYDCNLQQARKQTMERVAALSPYLVTDTEKLSLWRAQLHGMVDMSTRTRLSIHNNLFIGSIRGSGTPEQFKYWVKKGAVAVKQFYGCFAMTELGHGSNLKGLETTATYDQDSDQFIINTPHIGATKWWIGGAAHTSTHCVCFAKLIVHGKDYGTRNFVVPLRNVHDHSLKVGVSIGDIGKKMGRDGVDNGWIQFTNVRIPRQNMLMRYAKVSDTGVVTKPALDQLTYGALIRGRVSMIADSFHVSKRFLTIALRYACVRRQFGTSGDTKETKIIDYPYHQRRLLPLLAYCYAMKMGADEAQKTWIETTDRILALNPNDPAQKNDLEKAVTDTKELFAASAGMKAFTTWGCAKIIDECRQACGGHGYSGYNGFGQGYADWVVQCTWEGDNNVLCLSMGRGLVQSALQILAGKHVGASIQYVGDKSKISQNGQGTPREQLLSPEFLVEAFRTASRNNILRTTDKYQELVKTLNPDQAFEELSQQRFQCARIHTRQHLISSFYARIATAKDDIKPHLLKLANLFALWSIEEDTGIFLRENILTPGDIDLINSLVDELCVAVRDQVIGLTDAFGLSDFFINAPIGSYDGNVYEKYFAKVNQQNPATNPRPPYYESTLKPFLFREEEDDEICDLDE

SEQ ID NO: 23 - protein, 700 aa, Yarrowia lipolytica; identical to SEQ ID NO: 11

SEQ ID NO: 24 - protein, 700 aa, Yarrowia lipolytica
MISPNLTANVEIDGKQYNTFTEPPKALAGERAKVKFPIKDMTEFLHGGEENVTMIERLMTELERDPVLNVSGDYDMPKEQLRETAVARIAALSGHWKKDTEKEALLRSQLHGIVDMGTRIRLGVHTGLFMGAIRGSGTKEQYDYWVRKGAADVKGFYGCFAMTELGHGSNVAGLETTATYIQDTDEFIINTPNTGATKWWIGGAAHSATHTACFARLLVDGKDYGVKIFVVQLRDVSSHSLMPGIALGDIGKKMGRDAIDNGWIQFTNVRIPRQNMLMKYAKVSSTGKVSQPPLAQLTYGALIGGRVTMIADSFFVSQRFITIALRYACVRRQFGTTPGQPETKIIDYPYHQRRLLPLLAFTYAMKMAADQSQIQYDQTTDLLQTIDPKDKGALGKAIVDLKELFASSAGLKAFTTWTCANIIDQCRQACGGHGYSGYNGFGQAYADWVVQCTWEGDNNVLCLSMGRGLIQSCLGHRKGKPLGSSVGYLANKGLEQATLSGRDLKDPKVLIEAWEKVANGAIQRATDKFVELTKGGLSPDQAFEELSQQRFQCAKIHTRKHLVTAFYERINASAKADVKPYLINLANLFTLWSIEEDSGLFLREGFLQPKDIDQVTELVNHYCKEVRDQVAGYTDAFGLSDWFINAPIGNYDGDVYKHYFAKVNQQNPAQNPRPPYYESTLRPFLFREDEDDDICELDEE

SEQ ID NO: 25 - protein, 701 aa, Yarrowia lipolytica
MITPNPANDIVHDGKLYDTFTEPPKLMAQERAQLDFDPRDITYFLDGSKEETELLESLMLMYERDPLFNNQNEYDESFETLRERSVKRIFQLSKSIAMDPEPMSFRKIGFLGILDMGTYARLGVHYALFCNSIRGQGTPDQLMYWLDQGAMVIKGFYGCFAMTEMGHGSNLSRLETIATFDKETDEFIINTPHVGATKWWIGGAAHTATHTLAFARLQVDGKDYGVKSFVVPLRNLDDHSLRPGIATGDIGKKMGRDAVDNGWIQFTNVRVPRNYMLMKHTKVLRDGTVKQPPLAQLTYGSLITGRVQMTTDSHNVSKKFLTIALRYATIRRQFSSTPGEPETRLIDYLYHQRRLLPLMAYSYAMKLAGDHVRELFFASQEKAESLKEDDKAGVESYVQDIKELFSVSAGLKAATTWACADIIDKARQACGGHGYSAYNGFGQAFQDWVVQCTWEGDNTVLTLSAGRALIQSALVYRKEGKLGNATKYLSRSKELANAKRNGRSLEDPKLLVEAWEAVSAGAINAATDAYEELSKQGVSVDECFEQVSQERFQAARIHTRRALIEAFYSRIATADEKVKPHLIPLANLFALWSIEEDSALFLAEGYFEPEDIIEVTSLVNKYCGIVRKNVIGYTDAFNLSDYFINAAIGRYDGDVYKNYFEKVKQQYPPEGGKPHYYEDVMKPFLHRERIPDVPMEPEDIQ

SEQ ID NO: 26 - protein, 699 aa, Yarrowia lipolytica
MNNNPTNVILGGKEYDTFTEPPAQMELERAKTQFKVRDVTNFLTGSEQETLLTERIMREIERDPVLNVAGDYDADLPTKRRQAVERIGALARYLPKDSEKEAILRGQLHGIVDMGTRTRIAVHYGLFMGAIRGSGTKEQYDYWVAKGAATLHKFYGCFAMTELGHGSNVAGLETTATLDKDTDEFIINTPNSGATKWWIGGAAHSATHTACLARLIVDGKDYGVKIFIVQLRDLNSHSLLNGIAIGDIGKKMGRDAIDNGWIQFTDVRIPRQNMLMRYDRVSRDGEVTTSELAQLTYGALLSGRVTMIAESHLLSARFLTIALRYACIRRQFGAVPDKPETKLIDYPYHQRRLLPLLAYTYAMKMGADEAQQQYNSSFGALLKLNPVKDAEKFAVATADLKALFASSAGMKAFTTWAAAKIIDECRQACGGHGYSGYNGFGQAYADWVVQCTWEGDNNVLCLSMGRSLIQSCIAMRKKKGHVGKSVEYLQRRDELQNARVDNKPLTDPAVLITAWEKVACEAINRATDSFIKLTQEGLSPDQAFEELSQQRFECARIHTRKHLITSFYARISKAKARVKPHLTVLANLFAVWSIEEDSGLFLREGCFEPAEMDEITALVDELCCEAREQVIGFTDAFNLSDFFINAPIGRFDGDAYKHYMDEVKAANNPRNTHAPYYETKLRPFLFRPDEDEEICDLDE

SEQ ID NO: 27 - protein, 689 aa, Yarrowia lipolytica
MLSQQSLNTFTEPPVEMARERNQTSFNPRLLTYFLDGGEKNTLLMDRLMQEYERDPVFRNEGDYDITDVAQSRELAFKRIAKLIEYVHTDDEETYLYRCMLLGQIDMGAFARYAIHHGVWGGAIRGAGTPEQYEFWVKKGSLSVKKFYGSFSMTELGHGSNLVGLETTATLDKNADEFVINTPNVAATKWWIGGAADTATHTAVFARLIVDGEDHGVKTFVVQLRDVETHNLMPGIAIGDCGKKMGRQGTDNGWIQFTHVRIPRQNMLMRYCHVDSDGNVTEPMMAQMAYGALLAGRVGMAMDSYFTSRKFLTIALRYATIRRAFAAGGGQETKLIDYPYHQRRLLPLMAQTYAIKCTADKVRDQFVKVTDMLLNLDVSDQEAVPKAIAEAKELFSVSAGVKATTTWACAHTIDQCRQACGGHGYSAYNGFGRAYSDWVIQCTWEGDNNILCLSAGRALVQSNRAVRAGKPIGGPTAYLAAPAGSPKLAGRNLYDPKVMIGAWETVSRALINRTTDEFEVLAKKGLSTAQAYEELSQQRFLCTRIHTRLYMVKNFYERIAEEGTEFTKEPLTRLANLYAFWSVEEEAGIFLREGYITPQELKYISAEIRKQLLEVRKDVIGYTDAFNVPDFFLNSAIGRADGDVYKNYFKVVNTQNPPQDPRPPYYESVIRPFLFRKDEDEEICSLEDE

SEQ ID NO: 28 - protein, 906 aa, Candida viswanathii
MSPVDFKDKVVIITGAGGGLGKYYSLEFAKLGAKVVVNDLGGALNGQGGNSKAADVVVDEIVKNGGVAVADYNNVLDGDKIVETAVKNFGTVHVIINNAGILRDASMKKMTEKDYKLVIDVHLNGAFAVTKAAWPYFQKQKYGRIVNTSSPAGLYGNFGQANYASAKSALLGFAETLAKEGAKYNIKANAIAPLARSRMTESILPPPMLEKLGPEKVAPLVLYLSSAENELTGQFFEVAAGFYAQIRWERSGGVLFKPDQSFTAEVVAKRFSEILDYDDSRKPEYLKNQYPFMLNDYATLTNEARKLPANDASGAPTVSLKDKVVLITGAGAGLGKEYAKWFAKYGAKVVVNDFKDATKTVDEIKAAGGEAWPDQHDVAKDSEAIIKNVIDKYGTIDILVNNAGILRDRSFAKMSKQEWDSVQQVHLIGTFNLSRLAWPYFVEKQFGRIINITSTSGIYGNFGQANYSSSKAGILGLSKTMAIEGAKNNIKVNIVAPHAETAMTLTIFREQDKNLYHADQVAPLLVYLGTDDVPVTGETFEIGGGWIGNTRWQRAKGAVSHDEHTTVEFIKEHLNEITDFTTDTENPKSTTESSMAILSAVGGDDDDDDEDEEEDEGDEEEDEEDEEEDDPVWRFDDRDVILYNIALGATTKQLKYVYENDSDFQVIPTFGHLITFNSGKSQNSFAKLLRNFNPMLLLHGEHYLKVHSWPPPTEGEIKTTFEPIATTPKGTNVVIVHGSKSVDNKSGELIYSNEATYFIRNCQADNKVYADRPAFATNQFLAPKRAPDYQVDVPVSEDLAALYRLSGDRNPLHIDPNFAKGAKFPKPILHGMCTYGLSAKALIDKFGMFNEIKARFTGIVFPGETLRVLAWKESDDTIVFQTHVVDRGTIAINNAAIKLVGDKAKI

SEQ ID NO: 29 - protein, 472 aa, Candida viswanathii
MMWKFLIAIGLIFSYCCNAQLLDSLSFDNNWVNTHYIRTIDLSKGFVKETDLIQIKNINDKPQDEYYFVVNDGFDSIDELSIFSAFVGDQALEVEVDEVVPDKVFKLKLPVPIAPNSDLELRINFVYIDSLVSVPSKIAMDATQQLLYKTNKFPFSPYVTQEYTLALSGMSKGQEMDLHIDVEDTPGLPDLKPRVESQVLKYGPIAEDIPAFALKPMGLMYDHNRPLTKAVSLNRSIWLPASDINKVSIEEYYELTNTGAELDKGFSRVDWMKGRFESTRNHWALSHLEIPLLERGFDDYYYTDKVGVVSTHKIFKNHLLLQPRYPVFGGWKYNFTLGWSEELSKFLHKLHDNQDEYIIKFPILNSLRDVTYQDVYLEFYLPENAEFQNISSPIAFESISIENELSYLDVSKGHTKITVHYTNLFDDLHKLDVFVKYQYTQVAFIYKIAKISGFVFLGLVSYYLLGLLDLSI

SEQ ID NO: 30 - protein, 519 aa, Saccharomyces fibuligera
MKFGVLFSVFAAIVSALPLQEGPLNKRAYPSFEAYSNYKVDRTDLETFLDKQKEVSLYYLLQNIAYPEGQFNNGVPGTVIASPSTSNPDYYYQWTRDSAITFLTVLSELEDNNFNTTLAKAVEYYINTSYNLQRTSNPSGSFDDENHKGLGEPKFNTDGSAYTGAWGRPQNDGPALRAYAISRYLNDVNSLNEGKLVLTDSGDINFSSTEDIYKNIIKPDLEYVIGYWDSTGFDLWEENQGRHFFTSLVQQKALAYAVDIAKSFDDGDFANTLSSTASTLESYLSGSDGGFVNTDVNHIVENPDLLQQNSRQGLDSATYIGPLLTHDIGESSSTPFDVDNEYVLQSYYLLLEDNKDRYSVNSAYSAGAAIGRYPEDVYNGDGSSEGNPWFLATAYAAQVPYKLAYDAKSASNDITINKINYDFFNKYIVDLSTINSAYQSSDSVTIKSGSDEFNTVADNLVTFGDSFLQVILDHINDDGSLNEQLNRYTGYSTGAYSLTWSSGALLEAIRLRNKVKALA

SEQ ID NO: 31 - protein, 403 aa, Rattus norvegicus
MIHTNLKKKFSLFILVFLLFAVICVWKKGSDYEALTLQAKEFQMPKSQEKVAMGSASQVVFSNSKQDPKEDIPILSYHRVTAKVKPQPSFQVWDKDSTYSKLNPRLLKIWRNYLNMNKYKVSYKGPGPGVKFSVEALRCHLRDHVNVSMIEATDFPFNTTEWEGYLPKENFRTKVGPWQRCAVVSSAGSLKNSQLGREIDNHDAVLRFNGAPTDNFQQDVGSKTTIRLMNSQLVTTEKRFLKDSLYTEGILIVWDPSVYHADIPKWYQKPDYNFFETYKSYRRLNPSQPFYILKPQMPWELWDIIQEISADLIQPNPPSSGMLGIIIMMTLCDQVDIYEFLPSKRKTDVCYYHQKFFDSACTMGAYDPLLFEKNMVKHLNEGTDEDIYLFGKATLSGFRNIRC

SEQ ID NO: 32 - protein, 219 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQISF

SEQ ID NO: 33 - protein, 815 aa, Candida viswanathii
MTLLLKPTSELDATSRKIIERIQSNSPTFQHLFDLLLNLLPFFDKTVSLLGSIGYCDYEVAYVTYQTCIQVVGLMKPKTNSLNQDIFKGVQLQTRKRASTFKAILSYFAEPETQEEDPLLNRFKSLSGGGSKTKSSQDEVFHEWITSSELQRELSSKKVLLIDFRPRKDYLNNHIKYKDLVHIEPTQLETLLDSASDQDLETLVKKSAPYDQYHIFLERHKYDLIVVYNYNYGSESTDRLLGIIDVVSKPNPFTKLITILMNNKYISSRLKVKPLFLSGGVLNWYKTFGIEYLERTLVQNGVAHTSDNQYLKSFNDYVSTSKETPKTQVKTQNGDYIRPSQRKVNQFDPVPVKSGPTVFASAKVDLPPTPGSPAVSTPSPPRAPAPPTKTTSLTHVPEKEAKSPSPVTKEVTVSSKKSQFLELYTTGLVNLGNSCYMNCVVQCLAAAPQLTSFFFPTITESFSDHSYRQHINSNNKLGTKGELTTSFVELILNMLNNNGKAFSPTKFKRTMGSLSPSQQFLTYDQQDCIEFLNFLLDALHEDLNNVTITDPSERKLITDLSPEQEKSRETLPVRLASTIEWERYLKLNFSVIVDYFQGQHLSQLKCLECGFTSTTYNAFSILSLPIPQKLNNLGKVLLKDCLEEFVTTELLDDNNKWYCPQCKRFTRLTKKIAITRLPQVLIVNFNRFKMTNTGGFNKLETFVTYPVNEELDMTPYWPDVGSRINENSTMSIEMEQDLLQSFPIRNQTPPFKYKLFGVANHFGNLTTGHYTSYVYKHSDSKKTRNWCYFDDSKITYNVSPSQVVNKNAYCLFFQR

SEQ ID NO: 34 - protein, 421 aa, Candida viswanathii
MQLSLSVLSTVATALLSLTTAVDAKSHNIKLSKLSNEETLDASTFQEYTSSLANKYMNLFNAAHGNPTSFGLQHVLSNQEAEVPFVTPQKGGKYDAPLTNYLNAQYFTEIEIGTPGQPFKVILDTGSSNLWVPSQDCTSLACFLHSKYDHDASSTYKANGSEFSIQYGSGSMEGYISQDILTIGDLVIPKQDFAEATSEPGLAFAFGKFDGILGLAYDSISVNHIVPPVYNAINQGLLDKPQVSFYLGNTEKDENDGGLATFGGYDASLFQGKITWLPVRRKAYWEVSFEGIGLGDEYAELQKTGAAIDTGTSLITLPSSLAEIINAKIGATKSWSGQYQIDCAKRDELPDLTLTFAGHNFTLTAHDYILEVSGSCISVFTPMDFPKPIGDLAIIGDAFLRKYYSIYDLDKNAVGLAPSKA

SEQ ID NO: 35 - protein, 551 aa, Candida viswanathii
MGITNETQALLGGDSLSCLNKKKSNTKRNLSYLLNIITVSIIAYLCFFATHNHHNDSGIPKVDPHKKKNIIMMVTDGMGPASLSAARSFRQFRDKLAINDILTLDQYLIGSSRTRSSSSLVTDSAAGATAFSCALKSYNGAIGVSPDKSPCGTILEALKLQGYYTGLVVTTRITDATPAAFSAHVDYRFQEDLIAEHQLGEYPFGRAVDLILGGGRCHFLPTAQGGCRADDRNLIKESSDTWQYVGDRQQFDQLKGGKNVSLPLLGLLANTDIPYAIDRDEKEYPSLAEQVKVALTALSDATKDSDQGFFLLIEGSRIDHAGHHNDPTAQVREVLAYDEAFGEVIKFIDSTDVETVAISTSDHETGGLVVSRQVTPEYPDYIWYPEVLLNSTHSGDYLAHKIADYKNKDDTAKLTKFIKHEILETDLGVTDYTDKDVQAILDKVNDPANLLYVLNDIVSFRAQIGWTTHGHSAVDVNIYAHTNSPAIRAKLASAKAYHGLSGNHENIEIGAFMEEITGSNLSRVTELIKKTAHSPSLSKKEFSVDEFHGNV

SEQ ID NO: 36 - protein, 140 aa, Saccharomyces cerevisiae
MCCYCVCCTVSDFILYIVAFFFPPAAVLLRSGPCSSDFLLNVLLTLLGFLPGMLHAFYYITITSPLRNAEYVYYYQQGWVDSERNVPSNRPQNSQTPQNRPQQGSSARNVYPSVETPLLQGAAPHDNKQSLVESPPPYVP

SEQ ID NO: 37 - protein, 377 aa, Candida viswanathii
MRLKDIKLILIGILTISVTYFLISSFSGPRAYTTSDPNSSKMQFLRALESHPNWKETGLNFQPTKKLEVDDSSTPVRQQLAARFPYDPTQPFPKNIWQTWKVGIEDETFPKRYLKFQLSWDTKNPEYKHHVIPDDQCDELVAQLFEDVPDVARAYKVMPKSILKADFFRYLILFARGGVYTDIDTVGLKPIDTWMSNMELLWGEPNRAGLVVGIEADPDRPDWADWYARRIQFCQWTIQLKKGHPMLRELITKITDITLTREKRNELKKVLGKDEGGDIMNWTGPGIFTDTVFSYMNAILQAPEVITGKYKWDNIVDWKVFTGMQMPIAIDDVLVLPITSFSPDVSQMGSKSSTDPMAYAKHMFLGSWKDDGMPEME

SEQ ID NO: 38 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 39 - protein, 385 aa, Cannabis sativa
MNHLRAEGPASVLAIGTANPENILIQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSDSDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLDLKKEKFVDSRHVLSEHGNMSSSTVLFVMDELRKRSLEEGKSTTGDGFEWGVLFGFGPGLTVERVVVRSVPIKY

SEQ ID NO: 40 - protein, 391 aa, synthetic construct
  REGION 1..391: note = Cannabis sativa
MNHLRAEGPASVLAIGTANPENILIQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSDSDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLDLKKEKFVDSRHVLSEHGNMSSSTVLFVMDELRKRSLEEGKSTTGDGFEWGVLFGFGPGLTVERVVVRSVPIKYGRRAKL

SEQ ID NO: 41 - protein, 105 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRKLKPK

SEQ ID NO: 42 - protein, 111 aa, synthetic construct
  REGION 1..111: note = Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRKLKPKGRRAKL

SEQ ID NO: 43 - protein, 393 aa, Cannabis sativa
MGLSSVCTFSFQTNYHTLLNPHNNNPKTSLLCYRHPKTPIKYSYNNFPSKHTKSFHLQNKCSESLSIAKNSIRAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 44 - protein, 321 aa, synthetic construct
  REGION 1..321: note = Cannabis sativa
MAATTNQTEPPESDNHSVATKILNFGKACWKLQRPYTIIAFTSCACGLFGKELLHNTNLISWSLMFKAFFFLVAILCIASFTTTINQIYDLHIDRINKPDLPLASGEISVNTAWIMSIIVALFGLIITIKMKGGPLYIFGYCFGIFGGIVYSVPPFRWKQNPSTAFLLNFLAHIITNFTFYYASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 45 - protein, 398 aa, Cannabis sativa
MGLSLVCTFSFQTNYHTLLNPHNKNPKNSLLSYQHPKTPIIKSSYDNFPSKYCLTKNFHLLGLNSHNRISSQSRSIRAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 46 - protein, 394 aa, Cannabis sativa
MGLSSVCTFSFQTNYHTLLNPHNNNPKTSLLYRHPKTPIKYSYNNFPSKHCSTKSFHLQNKCSESLSIAKNSIRAATTNQTEPPESDNHSVATKILNFGKACWKLQRPYTIIAFTSCACGLFGKELLHNTNLISWSLMFKAFFFLVAVLCIASFTTTINQIYDLHIDRINKPDLPLASGEISVNTAWIMSIIVALFGLIITIKMKGGPLYIFGYCFGIFGGTVYSVPPFRWKQNPSTAFLLNFLAHIITNFTFYHASRAALGLPFELRPSFTFLLAFMKSMGSALALIKDASDVEGDTKFGISTLASKYGSRNLTLFCSGIVLLSYVAAILAGIIWPQAFNSNVMLLSHAILAFWLILQTRDFALTNYDPEAGRRFYEFMWKLYYAEYLVYVFI

SEQ ID NO: 47 - protein, 397 aa, Cannabis sativa
MELSSICNFSFQTNYHTLLNPHNKNPKSSLLSHQHPKTPIITSSYNNFPSNYCSNKNFHLQNRCSKSLLIAKNSIRTDTANQTEPPESNTKYSVVTKILSFGHTCWKLQRPYTFIGVISCACGLFGRELFHNTNLLSWSLMLKAFSSLMVILSVNLCTNIINQITDLDIDRINKPDLPLASGEMSIETAWIMSIIVALTGLILTIKLNCGPLFISLYCVSILVGALYSVPPFRWKQNPNTAFSSYFMGLVIVNFTCYYASRAAFGLPFEMSPPFTFILAFVKSMGSALFLCKDVSDIEGDSKHGISTLATRYGAKNITFLCSGIVLLTYVSAILAAIIWPQAFKSNVMLLSHATLAFWLIFQTREFALTNYNPEAGRKFYEFMWKLHYAEYLVYVFI

SEQ ID NO: 48 - protein, 400 aa, Cannabis sativa
MVFSSVCSFPSSLGTNFKLVPRSNFKASSSHYHEINNFINNKPIKFSYFSSRLYCSAKPIVHRENKFTKSFSLSHLQRKSSIKAHGEIEADGSNGTSEFNVMKSGNAIWRFVRPYAAKGVLFNSAAMFAKELVGNLNLFSWPLMFKILSFTLVILCIFVSTSGINQIYDLDIDRLNKPNLPVASGEISVELAWLLTIVCTISGLTLTIITNSGPFFPFLYSASIFFGFLYSAPPFRWKKNPFTACFCNVMLYVGTSVGVYYACKASLGLPANWSPAFCLLFWFISLLSIPISIAKDLSDIEGDRKFGIITFSTKFGAKPIAYICHGLMLLNYVSVMAAAIIWPQFFNSSVILLSHAFMAIWVLYQAWILEKSNYATETCQKYYIFLWIIFSLEHAFYLFM

SEQ ID NO: 49 - protein, 384 aa, Cannabis sativa
MELSLSLGGPTIFPRYRASYTSTKLTTHFSNFPSKFSTKNFHQTLSFYGPTRGSKSLLNTHQWRNSIRACAEAGAAGSNPVLNKVSDFRDACWRFLRPHTIRGTTLGSIALVARALIENPNLIKWSLLLKAFSGLLALICGNGYIVGINQIYDIGIDKVNKPYLPIAAGDLSVQSAWYLVILFAVAGLLTVGFNFGPFITSLYCLGLVLGTIYSVPPFRMKRFPVAAFLIIATVRGFLLNFGVYYATRAALGLTFEWSSAVAFITTFVTLFALVIAITKDLPDVEGDRKFQISTFATKLGVRNIAYLGSGLLLLNYIGAIAAAIYMPQAFKRNLMLPIHTILALSLVFQAWVLEQANYTKEAIAGFYRFIWNLFYVEYIIFPFI

SEQ ID NO: 50 - protein, 387 aa, Cannabis sativa
MAIALWLPRISRSTTRRFLKPSSSLTLFSVSHSHNYIVTSNRSPIPRLFTVPNQSHGREWVSVSEVRLGYVSHISTAGKSDENRSRDAQVADVSWIDLYLPRQIHPYVRLARLDKPIGTWLLAWPCMWSISLAANPGHLPDIKMMTLFGCGALLLRGAGCTINDLLDRDIDTMVERTKLRPVASGIITPFQGICFLGFQLLLGLGILLQLNNYSRILGASSLLLVFSYPLMKRLTFWPQAYLGLTFNWGALLGWAAVKGNIDPAIVLPLYASGVFWTLVYDTIYAHQDKEDDVRVGIKSTALRFGDLTKQWNMGFGAACISSLALSGYNAEIGWPFYASLVAASGQLAWQISTVDLSSRDDCNKKFVSNKWFGAIIFSGIVLARISS

SEQ ID NO: 51 - protein, 308 aa, Cannabis sativa
MSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLGAYYHITDVQRGLLKAFDSLEDG

SEQ ID NO: 52 - protein, 852 aa, Streptomyces
MSEAADVERVYAAMEEAAGLLGVACARDKIYPLLSTFQDTLVEGGSVVVFSMASGRHSTELDFSISVPTSHGDPYATVVEKGLFPATGHPVDDLLADTQKHLPVSMFAIDGEVTGGFKKTYAFFPTDNMPGVAELSAIPSMPPAVAENAELFARYGLDKVQMTSMDYKKRQVNLYFSELSAQTLEAESVLALVRELGLHVPNELGLKFCKRSFSVYPTLNWETGKIDRLCFAVISNDPTLVPSSDEGDIEKFHNYATKAPYAYVGEKRTLVYGLTLSPKEEYYKLGAYYHITDVQRGLLKAFDSLEDGMKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 53 - protein, 573 aa, synthetic construct
  REGION 1..573: note = Cannabis sativa
MFLKHIFVALAFALLADATPAQKRSPGFVALDFDIVKVQKNVTANDDAAAIVAKRQTNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 54 - protein, 543 aa, synthetic construct
  REGION 1..543: note = Cannabis sativa
MQLSLSVLSTVATALLSLTTAVDAKSHNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 55 - protein, 608 aa, synthetic construct
  REGION 1..608: note = Cannabis sativa
MQLSLSVLSTVATALLSLTTAVDAKSHNIKLSKLSNEETLDASTFQEYTSSLANKYMNLFNAAHGNPTSFGLQHVLSNQEAEVPFVTPQKGGNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 56 - protein, 523 aa, synthetic construct
  REGION 1..523: note = Cannabis sativa
MNPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRHGRRAKL

SEQ ID NO: 57 - protein, 545 aa, Cannabis sativa
MNCSAFSFWFVCKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGMSYISQVPFVVVDLRNMHSIKIDVHSQTAWVEAGATLGEVYYWINEKNENFSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGVGMYVLYPYGGIMEEISESAIPFPHRAGIMYELWYTASWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPHHH

SEQ ID NO: 58 - protein, 574 aa, synthetic construct
  REGION 1..574: note = Cannabis sativa
MFLKHIFVALAFALLADATPAQKRSPGFVALDFDIVKVQKNVTANDDAAAIVAKRQTNPQENFLKCFSEYIPNNPANPKFIYTQHDQLYMSVLNSTIQNLRFTSDTTPKPLVIVTPSNVSHIQASILCSKKVGLQIRTRSGGHDAEGMSYISQVPFVVVDLRNMHSIKIDVHSQTAWVEAGATLGEVYYWINEKNENFSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGVGMYVLYPYGGIMEEISESAIPFPHRAGIMYELWYTASWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPHHH

SEQ ID NO: 59 - protein, 544 aa, synthetic construct
  REGION 1..544: note = Cannabis sativa
MQLSLSVLSTVATALLSLTTAVDAKSHNPQENFLKCFSEYIPNNPANPKFIYTQHDQLYMSVLNSTIQNLRFTSDTTPKPLVIVTPSNVSHIQASILCSKKVGLQIRTRSGGHDAEGMSYISQVPFVVVDLRNMHSIKIDVHSQTAWVEAGATLGEVYYWINEKNENFSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGVGMYVLYPYGGIMEEISESAIPFPHRAGIMYELWYTASWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPHHH

SEQ ID NO: 60 - protein, 609 aa, synthetic construct
  REGION 1..609: note = Cannabis sativa
MQLSLSVLSTVATALLSLTTAVDAKSHNIKLSKLSNEETLDASTFQEYTSSLANKYMNLFNAAHGNPTSFGLQHVLSNQEAEVPFVTPQKGGNPQENFLKCFSEYIPNNPANPKFIYTQHDQLYMSVLNSTIQNLRFTSDTTPKPLVIVTPSNVSHIQASILCSKKVGLQIRTRSGGHDAEGMSYISQVPFVVVDLRNMHSIKIDVHSQTAWVEAGATLGEVYYWINEKNENFSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGVGMYVLYPYGGIMEEISESAIPFPHRAGIMYELWYTASWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPHHH

SEQ ID NO: 61 - protein, 525 aa, synthetic construct
  REGION 1..525: note = Cannabis sativa
MSNPQENFLKCFSEYIPNNPANPKFIYTQHDQLYMSVLNSTIQNLRFTSDTTPKPLVIVTPSNVSHIQASILCSKKVGLQIRTRSGGHDAEGMSYISQVPFVVVDLRNMHSIKIDVHSQTAWVEAGATLGEVYYWINEKNENFSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGVGMYVLYPYGGIMEEISESAIPFPHRAGIMYELWYTASWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPHHHGRRAKL

SEQ ID NO: 62 - protein, 545 aa, Cannabis sativa
MNCSTFSFWFVCKIIFFFLSFNIQISIANPQENFLKCFSEYIPNNPANPKFIYTQHDQLYMSVLNSTIQNLRFTSDTTPKPLVIVTPSNVSHIQASILCSKKVGLQIRTRSGGHDAEGLSYISQVPFAIVDLRNMHTVKVDIHSQTAWVEAGATLGEVYYWINEMNENFSFPGGYCPTVGVGGHFSGGGYGALMRNYGLAADNIIDAHLVNVDGKVLDRKSMGEDLFWAIRGGGGENFGIIAACKIKLVVVPSKATIFSVKKNMEIHGLVKLFNKWQNIAYKYDKDLMLTTHFRTRNITDNHGKNKTTVHGYFSSIFLGGVDSLVDLMNKSFPELGIKKTDCKELSWIDTTIFYSGVVNYNTANFKKEILLDRSAGKKTAFSIKLDYVKKLIPETAMVKILEKLYEEEVGVGMYVLYPYGGIMDEISESAIPFPHRAGIMYELWYTATWEKQEDNEKHINWVRSVYNFTTPYVSQNPRLAYLNYRDLDLGKTNPESPNNYTQARIWGEKYFGKNFNRLVKVKTKADPNNFFRNEQSIPPLPPRHH

SEQ ID NO: 63 - DNA, 567 bp, Candida viswanathii
  source 1..567: mol_type = other DNA
gaggatgaagaagacgaagacgaattggatgaagatgaagcgtatgagtattatgagtactgtcggacgttggaaggtggcagagttaagcccgagaaagcaaggaaggagtgggagatgatgagtgatgcggccaagaggatgtgaaggctgcgtatctgtttttgatagctggtggtagccgaatagaggaaggcaagcttgttcatattggatgatgatggtagatggtggctgccaaagtggttgtaaatagaaaaaagtgggtttgggtctgttgatagttagtggtggcggctgtctgtgattacgtcagcaagtagcacctcggcagttaaaacagcagcaacagaaaaaaaatgtgtgaaagtttgattcccccacagtctaccacacccagagttccatttatccataatatcacaagcaatagaaaaataaaaaattatcaacaaatcacaacgaaaagattctgcaaaattattttcacttcttcttttgacttcctcttcttcttgttaggttctttccatattttccccttaaacccatacacaacgcagccat

SEQ ID NO: 64 - DNA, 529 bp, synthetic construct
  misc_feature 1..529: note = PoX4_Promoter_CV
  source 1..529: mol_type = other DNA
gagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataa

SEQ ID NO: 65 - DNA, 861 bp, Candida viswanathii
  source 1..861: mol_type = other DNA
gtgccgacggagtaacaaaaatgttacagtgcgcactacttatcccgctaaggtgataaactgcaaaaacacaccgtcaaggagaagtcgacgttttgccgccacttgtgaagggaagaagagtcgttgagttgatgtaattaagctggcacgtagataccagaaggttctagagtagagcttgggtggtgtttggccctgtttggaccacggatagagatggagaatcccttggttagagcggagaggaaaaaattgaaactttgcatatcccacttcattatccttgatgtaaccgttttatggggtaattaaagtgtggaaaaataatcagggagacatattcccgatcaattgggtggtggtcgctcaatttctgtgagtagtaggctcagtggtgtgtattgggattggtagtagtctgtataagcagtgttatataacccattgcttgttgattcctattttgctggcaaaagtgacaactgtagttgtgagataatcctcggttattacgcctgggggggcagacagccaaagttgtgcccgtgcgacaatggcatcagaagaaacagaaaaaaaaaacacaggcatttttatccacatgcacactacccccactattcctgtctgcagtgtgcttgtgtgtggccccccgcagaatcaacagggcaaactctggagcctgaatctttatataaacttcaggcattggccccccttttcacaattcttcacatccaccattttttttcttctttcctaccatattagtttttttttattcttttcctacctatctgattattatcaaacatctggtcatcctcaaaagaaagaaagaaactataacaatcaatc

SEQ ID NO: 66 - DNA, 599 bp, Candida viswanathii
  source 1..599: mol_type = unassigned DNA
tggattatgtaatcacaggctgtttctccattcctgcatgtaggctggcccgcggtatcaaccatcgtcccgcttcttctggttttttttttttttccgctatgatatttttgatctcttgggggatttggtgggtctgccccccccgctactacaagctcaaacacccgaaaccttacaacacacacacacacatcccgcttagttgcgggttgaagaacgtgtattcccgtagggttaatggtgtgtccccccctagtcacccgcttctgccattctgggtttgccttcaaagctggcataaatgacgaaaaaaaagcacagcatcctgcacacaaccctgctcagtgtgacaggtggtggtgtaatagaaaacctcggcttaaaacctctggtcagagcatcaactgcaatcttgtctttttctgctgccctcacattctccccacacattcccaccctcaagatttcacaggcaaaaactgcaaatatatataaatctacaaccaattctttccccagatggaaaatctaatttttgttcaccctttttctttctcctgctatcactgctactgcacatattcaacaccacaacc

SEQ ID NO: 67 - DNA, 502 bp, synthetic construct
  misc_feature 1..502: note = TEF1_promoter_CV
  source 1..502: mol_type = other DNA
gactgggagaacagaccaaggatgcccatacaccattaataacaagcacaccttgataaatctctagtgttgacaaattggtgatttgaacatgactgtagagagagagacaagtaaccactgatggattggtggtggcaaataaccactttaaataaagaccaactcacacacaaagacagcaggtcgtgttcctatttccaattttcacaaggagaagaataaaaatttttcaatgagattaactaaagaaacagacaggcagcccacaagaagaagaagaagaagaggaagaggaagaagaagaagaagaagaagaagaagagaaaaaaaatttttccctctgcgttgcgttgggcttgggttgccagcacccaccatatataactctcatcaaatacccagtagagaaaaatttttcctccctctttttctttctttcttctccttcttttgctactctttctgtttttcatcaaaaagatatatataatcaatcatg

SEQ ID NO: 68 - DNA, 502 bp, Candida viswanathii
  source 1..502: mol_type = other DNA
accaaacgttactttttttttgcaatcggatggtatgggtctggggttcacctgttttgtaaagctacagaaggtggcatatttctctgatcaggtgttttttttttcggctgctgctgctcgtggtggtgtagtggtagtggtgtgtgtgtgtgtgtgtgcgtgcgtgtggaaggacgctttttgctctctgactcctcccaatcagaagttgctatagtggtgaaacaacaatggatgataatgccccgggcggtgcgtgtccgacacaaaccactacattttttagctgggagcctactgccactacgacccacccacccatggtcaacaaaaaaattctgacaaattataaaataacccttgaattcccccttggaaaaatttttggtatttctctctctcttttcctttccctcttctttttctctccatcaatcaattgacgttcagtaactcaattaattacatcacatccctcaattaaagaatttaaacaatg

SEQ ID NO: 69 - DNA, 1006 bp, synthetic construct
  misc_feature 1..1006: note = POX2_Promoter_Yl
  source 1..1006: mol_type = other DNA
acgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgcttgagtatcgaagtcttgtacctccctgtcaatcatggcaccactggtcttgacttgtctattcatactggacaagcgccagagttaagcttgtagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggaggcctaattttgacgtgcaaggggcgaattggggcgagaaacacgtcgtggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacgacgccatga

SEQ ID NO: 70 - DNA, 1004 bp, Yarrowia lipolytica
  source 1..1004: mol_type = other DNA
aaacttctccgagtctgtgccttcaggtgggcatagttgatgggtgttttgaagttaatagtggggaagaactatggcaaacaagcagatgcaggcaccttgtaactgcagaccggttcttgtctaccgactccgctgcacctgtgccgcggtacatgtcgtcacaggctgcggggttcggaggcccccttgcaacctcctttgatagttgctatggccccaaagagttatacgagatagacccacagatctacttgactgttgtcacagaacctgctaggtttgcttattgtacccgctttgtagctactgtacaacgacaacgtcaaaaattgagacgcgaacaaactccagatgcagaacccaaacctctctctcagagtttcgagtgcttctacctcacagtaaagtggaggtggacctgcaagggaattcagtcacaaggccccgaatgtctccgaaactccaatcggaccgtttaaacagactaatatcacgtcattgattgatattagcatccggcaagagccgcaaggttatctcctcaccaatgagcctgttgtacggctcattccgcatctgcggctgattcagtttcgagtggggatggtagacttcattgcagcattcctaaccttctacttggtccgtggagatgtcatggacatcgattttgggctgagaagccttttgacgatgttgatatcactgaccgctaatttactctggcagtttctccggctctcgaggcatcgtcgatcaccaaacactatctgctagtctaaatgtccgacacgacagcttttgatcgccgtgaacggcgcagacctcatgcaccatgcaccagggccaaatcaattacgggtcgcttagcgttgcagtcggggcattatggtggaagttccgatacggcacagacacattccatagtggggggattggattataaaagggccatagaaagccctcaattgatacccaagtaccagctctcctcactatga

SEQ ID NO: 71 - DNA, 1367 bp, synthetic construct
  misc_feature 1..1367: note = ICL1_Promoter_Yk
  source 1..1367: mol_type = other DNA
gaccccctccttttgccagtatatccaccgcaacacccaccatgagcgacatctgataccgtgccgcgaccactaccccaaataagctccaactaatatgccgaggcaggtgggaaactatgcactccagacgacgctgtagaagcacatggaaggtgcggaggcggtggcaacgaggggcatgagccatcaacgagtaaccacagacaaggcaaggggggaaacgcgaccggaatctctcgcggtcacgtgacccgcccgggttccactcgtccatgttgtgtctctggtgtcttcggccgactcgcattggttaaacttccaccaccgcaatcacgtcccactggccaaactttttctgctttctctgactttttctggccaaaaggcaacgtcggaaagggtcgggaggattcggaaccgacgaaaatcggccggctccagcgggggtagttcggcagtcctggtgggagctctaggggagctgtggtctgtgtagggcgcgggtccgggtttgttgggtgtcaaatcacgtgtttttgcccccccgctgagccggactccgacaaccgtgtctccaacggcctgactaagctgctcccagcactctgccgtagcgttggtctgtcctgtcgcactctgttcaaagacagaagaaagaaaaagctaacctccacgtcagagacaatggtagaaggcttgttccttgcaaccgaggagagtgagtgttctcggcacgagcatcatgggcgatctggagggtatttttgaggggaaaaaacgggatcaggacaaacagaggccacagaccgggaatctgggccccaaaacggccttttcccgtcgcaaaaccggtctacatacaccccttcggcccgccacaggccggtgtgaaaaaccctaaagcttgcttcaaaccagacggacgcacagcaagacacatcatgaagagtcacctgcagtatatatagatctggggattcccagtagactgaccaagcatacaaaagtgagtatccaacagcgacacgtgagatggcagagacacagagacgtgtctacatggttggacaagtctccacattcgccagagacgtatccacatacaaacacaatctcacagctgatctgctcctgtgacagcacagtacatgttagtggatgaggtgttgtgtagtgggttaaatgggtggactgattcagtggcatcggtggcgacaccctctactcttcatgtcgtcacctaccgttcggaatcccaattatctgatgaactaaacgatttctggccaaaacacaattttgccaaagaagtcgttctcaccaatgcaagtgtcacatcaaacatctgtcccgtactaacccag

SEQ ID NO: 72 - DNA, 1004 bp, synthetic construct
  misc_feature 1..1004: note = POT1_Promoter+Yl
  source 1..1004: mol_type = other DNA
ccacaataccccacagtgtgcatatcaaacctaccggttgttgctctctccagccttactaagaaggaggcgacgtggcagtggctcgcgggaggatcggcgggaaactccgggatatccgtcgagagtttacacgtgaatgggcagcgcaatccgttgacgacgatacgactggcaaagtagcgacgatacctgccagacaggtgacatgtgcaggccgcactaacaaggaaacgggcgctggggggggcgggcttctagactttgcccttgaacaggaatctagtgggggcttgtctttccgccaatgggggagcgcctgttgagcgaccgtgcatgctggaacgccaagtgtatgtacagctggtgttctcgcagcggtatgtgacgggacttacatctctcgttttttcatgaccacgttttcacaggctcggaggtacgttaaagttttgaaggctgcatctgaaccgaggtatgggggagtttgaggagcaacagtgttggggctgagggggccaagatcggggcaagcagaggtcttagatcaattgtgggaatcccaaagggctcgttatcacctttttccacccaattcgggtcccaattgatccactactggcttgcccaagttaccccagaaatgccgccccggatttctccaaaaacctaataagcttcatggaacttggtggaagtgactttctacagagtggagagaaccgtggacacgtggcaatggcgctgaccgtgtccccgagccgaatcgacgtgaggggagaacggagtatctgcggtcatgtgaccttccagagcggcgtcgccagtgtgcacgcggtgacccccagtttggttctctgtcacacgcatactacctcggctctccacatgctgaactttatctttcgtggggatcataccgaaagttgcaactaccaggtgtatataaagcctggtagactccccccactttggacctcatccaaccaagacacacaaaaatg

SEQ ID NO: 73 - DNA, 174 bp, Candida viswanathii
  source 1..174: mol_type = other DNA
gagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgac

SEQ ID NO: 74 - DNA, 200 bp, Candida viswanathii
  source 1..200: mol_type = other DNA
agctccaggcttgttatgactctagagagaagtgtgtgtgtttgcgtttgttttactatacattcaacatgttctttttcttttttgatatttattccaactataattatacacagattcgtatatactttactttaccctctttcgtagttttttaatttgatgatttttgagtttcatatccaaggtcaaaacccgac

SEQ ID NO: 75 - DNA, 251 bp, Yarrowia lipolytica
  source 1..251: mol_type = other DNA
taggcaattaacagatagtttgccggtgataattctcttaacctcccacactcctttgacataacgatttatgtaacgaaactgaaatttgaccagatattgttgtaaatagaaaatctggcttgtaggtggcaaaatgcggcgtctttgttcatcaattccctctgtgactactcgtcatccctttatgttcgactgtcgtatttcttattttccatacatatgcaagtgagatgcccgtgtccgaattc

SEQ ID NO: 76 - DNA, 308 bp, synthetic construct
  misc_feature 1..308: note = TDH3_terminator_YL
  source 1..308: mol_type = other DNA
cttccgagtagctatccgaagatcaagagcgaagcaagttgtaagtccaggacatgtttcccgcccacgcgagtgatttataacacctctcttttttgacacccgctcgccttgaaattcatgtcacataaattatagtcaacgacgtttgaataacttgtcttgtagttcgatgatgatcatatgattacattaatagtaattactgtatttgatatatatactaattacaatagtacatattagaacatacaatagttagtgccgtgaagtggcttaaaataccgcgagtcgattacgtaatatta

SEQ ID NO: 77 - DNA, 2133 bp, synthetic construct
  misc_feature 1..2133: note = LEU2_Marker_Yl
  source 1..2133: mol_type = other DNA
aacgtaccactgtcctccactacaaacacacccaatctgcttcttctagtcaaggttgctacaccggtaaattataaatcatcatttcattagcagggctgggccctttttatagagtcttatacactagcggaccctgccggtagaccaacccgcaggcgcgtcagtttgctccttccatcaatgcgtcgtagaaacgacttactccttcttgagcagctccttgaccttgttggcaacaaagtctccgacctcggaggtggaggaggagcctccgatatcggcggtagtgataccagcctcgacggactccttgacggcagcctcaacagcgtcaccggcgggcttcatgttaagagagaacttgagcatcatggcggcagacagaatggtggcaatggggttgaccttctgcttgccgagatcgggggcagatccgtgacagggctcgtacagaccgaacgcctcgttggtgtcgggcagagaagccagagaggcggagggcagcagacccagagaaccggggatgacggaggcctcgtcggagatgatatcgccaaacatgttggtggtgatgatgataccattcatcttggagggctgcttgatgaggatcatggcggccgagtcgatcagctggtggttgagctccagctgggggaattcgtccttgaggactcgggtgacggtctttcgccaaagtcgagaggaggccagcacgttggccttgtcaagggaccacacgggaagaggggggttgtgctgaagggccaggaaggcggccattcgggcaattcgctcaacctcaggaacggagtaagtctcagtgtcggaagcgacgccagatccgtcatcctcctttcgctctccaaagtagatacctccgacgagctctcggacaatgatgaagtcggtgccctcaacgtttcggatgggggagagatcggcgagcttgggcgacagcagctggcagggtcgcaggttggcgtacaggttcaggtcctttcgcagcttgagaagaccctgctcgggtcgcacgtcggttcgtccgtcgggagtggtccatacggtgttggcagcgcctccgacagcaccgagcataatagagtcagcctttcggcagatgtcgagagtagcgtcggtgatgggctcgccctccttctcaatggcagctcctccaatgagtcggtcctcaaacacaaactcggtgccggaggcctcagcaacagacttgagcaccttgacggcctcggcaatcacctcggggccacagaagtcgccgccgagaagaacaatcttcttggagtcagtcttggtcttcttagtttcgggttccattgtggatgtgtgtggttgtatgtgtgatgtggtgtgtggagtgaaaatctgtggctggcaaacgctcttgtatatatacgcacttttgcccgtgctatgtggaagactaaacctccgaagattgtgactcaggtagtgcggtatcggctagggacccaaaccttgtcgatgccgatagcgctatcgaacgtaccccagccggccgggagtatgtcggaggggacatacgagatcgtcaagggtttgtggccaactggtaaataaatgatgactcaggcgacgacggaattcgacagcaactactcctttcaccaaccatgtgcattttagctcgaataacattcacaggcttggtgatctacatccatggtgtctggccgattaccgtggtgttttggcagtaacgagaatattgagtgaactcttcccatcaccaataaagactcatactacaatcacgagcgcttcagctgccactatagtgttggtgacacaatacccctcgatgctgggcattactgtagcaagagatattatttcatggcgcattttccagtctacctgactttttagtgtgatttcttctccacattttatgctcagtgtgaaaagttggagtgcacacttaattatcgccggttttcggaaagtactatgtgctcaaggttgcaccccacgttacgtatgcagcacattgagcagcctttggaccgtggagataacggtgtggagatagcaacgggtagtcttcgtattaattcaatgcattgttagttttatatgatatggtgtcga

SEQ ID NO: 78 - DNA, 1142 bp, synthetic construct
  misc_feature 1..1142: note = URA3_Marker_Yl
  source 1..1142: mol_type = other DNA
tttctaatttggaccgatagccgtatagtccagtctatctataagttcaactaactcgtaactattaccataacatatacttcactgccccagataaggttccgataaaaagttgtgcagactaaatttatttcagtctcctcttcaccaccaaaatgccctcctacgaagcgcgagctaacgtccacaagtccgcctttgccgcccgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggaactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggatgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagatgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtcatttaactgtgtatatagagagcgtgcaagtatggagcgcttgttcagcttgtatgatggtcagacgacctgtctgatcgagtatgtatgatactgcacaacctg

SEQ ID NO: 79 - protein, 9 aa, synthetic construct
  REGION 1..9: note = FLAG
DYKDDDDKG

SEQ ID NO: 80 - protein, 14 aa, paramyxovirus simian parainfluenza virus 5
GKPIPNPLLGLDST

SEQ ID NO: 81 - protein, 10 aa, Homo sapiens
EQKLISEEDL

SEQ ID NO: 82 - protein, 11 aa, Herpes simplex virus
QPELAPEDPED

SEQ ID NO: 83 - protein, 9 aa, Vesicular stomatitis virus
YPYDVPDYA

SEQ ID NO: 84 - protein, 261 aa, Influenza
MTKFIGCIDLHNGEVKQIVGGTLTSKKEDVPKTNFVSQHPSSYYAKLYKDRDVQGCHVIKLGPNNDDAAREALQESPQFLQVGGGINDTNCLEWLKWASKVIVTSWLFTKEGHFQLKRLERLTELCGKDRIVVDLSCRKTQDGRWIVAMNKWQTLTDLELNADTFRELRKYTNEFLIHAADVEGLCGGIDELLVSKLFEWTKDYDDLKIVYAGGAKSVDDLKLVDELSHGKVDLTFGSSLDIFGGNLVKFEDCCRWNEKQG

SEQ ID NO: 85 - protein, 5 aa, synthetic construct
  VARIANT 3: note = Xaa = Any Amino Acid
  REGION 1..5: note = Cysteine-rich tag
CCXCC

SEQ ID NO: 86 - protein, 6 aa, synthetic construct
  REGION 1..6: note = VSV-G
CCPGCC

SEQ ID NO: 87 - protein, 6 aa, synthetic construct
  REGION 1..6: note = polyhistidine element
CCPGCC

SEQ ID NO: 88 - protein, 261 aa, Saccharomyces cerevisiae; identical to SEQ ID NO: 84

SEQ ID NO: 89 - protein, 6 aa, Cricetulus griseus
LVPRGS

SEQ ID NO: 90 - protein, 5 aa, synthetic construct
  REGION 1..5: note = enterokinase
DDDDK

SEQ ID NO: 91 - protein, 7 aa, synthetic construct
  REGION 1..7: note = TEV protease
ENLYFQG

SEQ ID NO: 92 - protein, 8 aa, synthetic construct
  REGION 1..8: note = PreScission protease
LEVLFQGP

SEQ ID NO: 93 - DNA, 2130 bp, synthetic construct
  misc_feature 1..2130: note = CvACO1P Gene
  source 1..2130: mol_type = other DNA
atgacagaagtagtagatagagcaagttccccagcaagtccaggatctacgaccgccgccgcagacggtgctaaggtggcggtggagccacgcgtagatgtagcggcccttggcgagcagttgctagggcgatgggctgacatcagattgcacgcacgagacttagcaggccgcgaagtggtccaaaaggttgaaggacttacgcacactgagcatcggagtagagtctttggacagttgaagtacttggtagacaacaatgctgttcacagagcttttccctccaggctaggtggatcagatgaccatggcggtaatatagctggattcgaggaattagttactgctgatccatcattgcaaataaaggccggcgttcagtggggtttgtttggttctgcagtgatgcacttgggaacccgtgaacatcatgacaagtggttgccaggtattatgtcgttagaaataccggggtgtttcgcgatgacagaaaccgggcacggtagcgacgtggcctctattgctacaacagcaacttatgatgaggaaacccaagagtttgttattgataccccgttcagagccgcttggaaagattatatcggtaatgcagcgaacgatggtttggcggcagttgttttcgcacaattaatcacgaggaaagtgaaccatggtgtacacgccttttacgtggatctcagagatcctgcgactggagacttcctacccggaataggaggagaggacgatggaatcaaggggggattgaatggcattgacaacggtagactacattttacgaacgtacgcattcctagaactaatcttcttaacagatatggcgatgtggcggtcgacggcacatacctgtcgaccatcgaatcaccagggcgccggttctttacgatgcttggtactctagtccagggtagagttagtctcgatggagcagctgtcgctgcactgaaggtcgcattgcaaagtgcaattcactacgctgcggagaggagacaatttaatgcgacttcacctactgaagaagaggtccttcttgattatcagaggcatcaaaggagactctttacacgacttgcaacgacgtacgccgcatctttcgcccacgagcagctattgcaaaagttcgatgatgtcttttcaggggcacatgataccgacgccgaccggcaggacttggaaaccctagccgccgctttgaagccattgagcacatggcatgcacttgacacgttacaagaatgcagagaggcctgtgggggggccggatttttgatagaaaaccgtttcgcgagcttgcgtgctgacttggacgtttacgtcacattcgagggtgataacacagttttattgcaattggttgctaaacggctcttggcagactacgcaaaagagttcagaggggccaacttcggcgttcttgccaggtatgtggttgaccaagccgcgggagtggcgctccaccgaacaggactaaggcaagtcgctcaatttgttgcagacagcgggtccgttcagaagtcggctcttgcgcttcgcgatgaagagggtcaacgaacattgttaacggacagagtacagagcatggttgccgaagtgggggctgccttgaaaggcgcaggcaaattaccccaacatcaagcagctgcattgttcaaccaacaccagaacgaacttattgaggctgcccaggcccatgcagaactcctccaatgggaggcatttacagaagctctcgctaaagtcgacgatgctggtacaaaggaagtgcttactcgattgcgagatctctttggtttgtccttgattgaaaaacacttgctgtggtatcttatgaatggacgtttgtccatgcaaagaggcaggacagttggaacttacattaatcgtttacttgtcaagatccgtccacacgcactagacttggttgatgccttcggttacggcgcggagcatttgcgtgctgctatcgccaccggagcggaagcaacccgacaggatgaagcccgaacgtattttagacaacaacgggcatcgggactggccccggccgatgaaaagaccttactcgctatcaaagctggtaaatcaagagggcgaagggcaaagctatga

SEQ ID NO: 94 - DNA, 2163 bp, synthetic construct
  misc_feature 1..2163: note = CvHXS1 Gene
  source 1..2163: mol_type = other DNA
atgggaaaaaattataaatctttggactcagttgtggctagtgacttcattgcacttgggatcacatcagaagttgctgagacattgcacggacgcttggcagagatagtttgcaactacggcgccgcaacacctcagacctggattaacatcgcaaaccatattctaagtccagatcttccatttagtctccatcagatgttgttctacggttgttataaggactttggtccagcacccccagcttggataccagaccccgaaaaagtaaagtccacgaacttaggtgccttgttagaaaagcggggaaaggagtttctaggcgttaagtataaggacccaataagtctgttttctcacttccaggagtttagcgttcgaaatccggaagtctactggcggacggtacttatggatgaaatgaagatactgttcagcaaagatcccgaatgtatcctcagacgcgacgacattaacaacccagggggctctgagtggctaccaggtggatatctcaacctggccaagaactgtttgaatgtaaatagtaacaaaaaacttaacgacactatgatagtgtggagagatgaaggaaatgacgatctcccattgaataaattgactcttgatcaattacgaaaacgagtctggttggttggatacgccctagaagagatgggccttgagaagggatgtgcgattgcaattgacatgcccatgcacgtagatgcggttgtgatctatttagctatcgtcttggcaggctacgtcgttgtctccattgcagattcattctcagcaccggaaatttccacaagattgcgtctatcaaaggctaaggctatttttacacaagatcatatcatccgagggaaaaagcgtatacctttgtacctgcgtgtcgtcgaggccaagtctccgatggcaatagttatcccgtgttcgggttcaaatattggtgcggaattgcgggatggtgatattctgtgggattacttcttagaacgcgcaaaggaatttaagaactgcgaatttacagcccgtgaacagccagtggacgcgtacacaaatattttgttctcatcgggaaccaccggagagccaaaggcgataccatggactcaagctacgcctctcaaggcggctgctgatggttggtcacacttggacattagaaagggtgacgtaattgtatggcctaccaatttggggtggatgatggggccttggttggtctatgcttcactccttaacggggcaagcatcgcattgtataacggatctccactagtgtccggctttgccaaattcgttcaagatgcgaaagttactatgctaggagttgtcccctccatcgtacgaagctggaaaagcactaattgcgttagtgggtacgattggtctacaatcagatgcttctcctcatcgggtgaggcatcgaatgtcgatgaatacttatggctaatgggaagggctaactacaaaccggtcatcgaaatgtgcggtggcacagagatcgggggtgccttcagcgccggttcgtttttacaagcccaatctttgagtagcttctcatcccaatgtatgggatgcaccttgtacattctcgacaagaatggctacccgatgccaaagaacaagccgggtataggtgaattggccttgggacccgtgatgttcggtgcttccaagactttacttaacggaaaccatcatgacgtttatttcaaaggcatgcccaccttgaacggagaagtcttgaggagacacggagatatcttcgaactcacttcgaacggctattatcacgctcatggtagagcagatgacacgatgaatatcggggggattaaaatttcctcaatcgagattgaaagggtgtgtaatgaagttgacgatagagtgtttgagactacggccattggagtgcctccattgggcggaggtccagagcagctcgttatcttttttgttcttaaggacagcaatgatacgaccatcgacctaaaccaattgcgacttagttttaatcttgggttacaaaagaaattgaacccactttttaaggtgacgagggttgtgccactttcgctgttgcctaggacagccaccaacaaaataatgagaagagtgcttagacagcaatttagtcatttcgagtga

SEQ ID NO: 95 - DNA, 1158 bp, synthetic construct
  misc_feature 1..1158: note = CvTKS1 Gene
  source 1..1158: mol_type = other DNA
atgaatcatttaagagcagaaggacccgcatcagtgttagcgataggtacagctaacccagagaatatcttaatccaagatgaatttcctgactactatttccgtgttactaaatcggaacatatgactcaacttaaagagaagttccggaaaatctgcgataaatccatgatccgaaagagaaactgtttccttaacgaagaacatctcaagcaaaacccgaggttggtagagcacgaaatgcagaccttggatgctaggcaggacatgttggtggtcgaagtgccaaaactcggcaaggacgcgtgcgctaaggcaatcaaggagtggggtcaaccgaagtctaaaatcacgcatctaatatttacatctgcactgacaaccgacatgccgggtgccgattatcactgcgccaagctacttggattgagtccactggttaagagagttatgatgtatcaattggggtgttacggagggggcacagtcctcagaattgctaaggatattgcggaaaataacaagggcgcgagggtccttgctgtatgttgtgatattatggcctgtttgtttcgcgggccctcggattcagatttggaattgcttgtcggacaggcaatttttggtgacggggccgcagcagtcatagtgggagccgaaccagacgaaagcgtgggtgaaagaccaatctttgagttggttctgaccggacaaacgatcttacctaactcggaaggtacgattggaggacatattagagaagccggcctaattttcgatcttcacaaagacgttccaatgttaatctccaataacatagaaaagtgcttgatagaagcatttactcccattggtattagtgactggaacagcattttctggatcacccaccctggaggaaaagctatactcgataaggttgaagagaaactcgacttgaaaaaggagaaattcgttgactcacgacatgtgttatcagagcacgggaatatgagttcatccacagtcttgttcgtaatggatgaattgcgaaaacgctctcttgaggagggaaagagcacaaccggtgacgggtttgagtggggcgtgctattcggttttggcccaggtttgactgtcgagcgggttgttgttcgtagtgtaccaattaagtactga

SEQ ID NO: 96 - DNA, 1176 bp, synthetic construct
  misc_feature 1..1176: note = CvTKS1P Gene
  source 1..1176: mol_type = other DNA
atgaatcatttaagagcagaaggacccgcatcagtgttagcgataggtacagctaacccagagaatatcttaatccaagatgaatttcctgactactatttccgtgttactaaatcggaacatatgactcaacttaaagagaagttccggaaaatctgcgataaatccatgatccgaaagagaaactgtttccttaacgaagaacatctcaagcaaaacccgaggttggtagagcacgaaatgcagaccttggatgctaggcaggacatgttggtggtcgaagtgccaaaactcggcaaggacgcgtgcgctaaggcaatcaaggagtggggtcaaccgaagtctaaaatcacgcatctaatatttacatctgcactgacaaccgacatgccgggtgccgattatcactgcgccaagctacttggattgagtccactggttaagagagttatgatgtatcaattggggtgttacggagggggcacagtcctcagaattgctaaggatattgcggaaaataacaagggcgcgagggtccttgctgtatgttgtgatattatggcctgtttgtttcgcgggccctcggattcagatttggaattgcttgtcggacaggcaatttttggtgacggggccgcagcagtcatagtgggagccgaaccagacgaaagcgtgggtgaaagaccaatctttgagttggttctgaccggacaaacgatcttacctaactcggaaggtacgattggaggacatattagagaagccggcctaattttcgatcttcacaaagacgttccaatgttaatctccaataacatagaaaagtgcttgatagaagcatttactcccattggtattagtgactggaacagcattttctggatcacccaccctggaggaaaagctatactcgataaggttgaagagaaactcgacttgaaaaaggagaaattcgttgactcacgacatgtgttatcagagcacgggaatatgagttcatccacagtcttgttcgtaatggatgaattgcgaaaacgctctcttgaggagggaaagagcacaaccggtgacgggtttgagtggggcgtgctattcggttttggcccaggtttgactgtcgagcgggttgttgttcgtagtgtaccaattaagtacggaagaagggcaaagttgtga

SEQ ID NO: 97 - DNA, 318 bp, synthetic construct
  misc_feature 1..318: note = CvOAC1 Gene
  source 1..318: mol_type = other DNA
atggcagtcaaacacctaatagttctcaaatttaaagacgagattactgaagctcagaaggaagagttctttaagacatatgttaacttagtcaacatcatccccgcgatgaaggacgtctactggggcaaggatgtgacgcaaaaaaataaggaagaaggatacacacatatcgttgaggtgacctttgagagtgtggaaactattcaagattatattattcacccagcccatgtagggttcggtgacgtttatcgatcattctgggaaaagttgcttatatttgattacaccccaagaaaattgaagcctaagtga

SEQ ID NO: 98 - DNA, 336 bp, synthetic construct
  misc_feature 1..336: note = CvOACP1 Gene
  source 1..336: mol_type = other DNA
atggcagtcaaacacctaatagttctcaaatttaaagacgagattactgaagctcagaaggaagagttctttaagacatatgttaacttagtcaacatcatccccgcgatgaaggacgtctactggggcaaggatgtgacgcaaaaaaataaggaagaaggatacacacatatcgttgaggtgacctttgagagtgtggaaactattcaagattatattattcacccagcccatgtagggttcggtgacgtttatcgatcattctgggaaaagttgcttatatttgattacaccccaagaaaattgaagcctaagggaagacgagctaagttgtga

SEQ ID NO: 99 - DNA, 1188 bp, synthetic construct
  misc_feature 1..1188: note = CvPTS1 Gene
  source 1..1188: mol_type = other DNA
atgggtttatcgtcagtgtgcactttttcttttcaaacaaactaccacaccctcctaaaccctcacaataataacccaaaaacctccttgctatgttacagacatccaaagacaccgatcaagtattcatacaacaattttcccagtaaacattgctcaacgaagtccttccacttgcaaaacaaatgcagcgaatcattgtcgatagctaaaaactcgatacgtgcggcaaccactaaccaaactgagccaccagagagcgataatcattcagtcgccaccaagattttgaactttggaaaagcctgttggaaacttcaaaggccttacaccattatcgcatttaccagttgcgcatgtggtttgttcgggaaggaattattacacaacacaaatttgatcagctggagcctaatgtttaaggcatttttcttcttagttgcaattttgtgtatagcttcgtttacaacgaccattaatcagatttacgaccttcacatcgatcggatcaataaaccagacttgccccttgcctctggggaaatctctgtaaatactgcatggatcatgctgataatcgtggctttgtttggattgattattacaattaagatgaaggggggtccattatatatattcgggtactgcttcggcattttcggtggtatcgtttactccgtcccaccctttagatggaaacagaaccccagtacggcctttctactcaatttcttggctcatatcatcacaaacttcacattctattatgcaagccgagcggcgcttggtttgccgttcgaactcagaccgagttttacatttctccttgccttcatgaaactgatgggactggcccttgcattgatcaaggatgcgtcagatgtcgaaggcgacactaagttcggcattctgacgcttgcttccaagtatggaagtagaaatctaacgcttttttgttcaggaatagtgctacttagttatgttgctgctatactcgctggcattatttggcctcaggccttcaactctaacgtaatgttgttatcccatgctattttggcgttctggttgatcttgcaaacgcgagattttgcactcactaactacgacccagaggcaggaaggcgcttttacgagtttatgtggaagttgtattatgccgaatacttggtttatgttttcatttga

SEQ ID NO: 100 - DNA, 966 bp, synthetic construct
  misc_feature 1..966: note = CVPTS1dN Gene
  source 1..966: mol_type = other DNA
atggcggcaaccactaaccaaactgagccaccagagagcgataatcattcagtcgccaccaagattttgaactttggaaaagcctgttggaaacttcaaaggccttacaccattatcgcatttaccagttgcgcatgtggtttgttcgggaaggaattattacacaacacaaatttgatcagctggagcctaatgtttaaggcatttttcttcttagttgcaattttgtgtatagcttcgtttacaacgaccattaatcagatttacgaccttcacatcgatcggatcaataaaccagacttgccccttgcctctggggaaatctctgtaaatactgcatggatcatgctgataatcgtggctttgtttggattgattattacaattaagatgaaggggggtccattatatatattcgggtactgcttcggcattttcggtggtatcgtttactccgtcccaccctttagatggaaacagaaccccagtacggcctttctactcaatttcttggctcatatcatcacaaacttcacattctattatgcaagccgagcggcgcttggtttgccgttcgaactcagaccgagttttacatttctccttgccttcatgaaactgatgggactggcccttgcattgatcaaggatgcgtcagatgtcgaaggcgacactaagttcggcattctgacgcttgcttccaagtatggaagtagaaatctaacgcttttttgttcaggaatagtgctacttagttatgttgctgctatactcgctggcattatttggcctcaggccttcaactctaacgtaatgttgttatcccatgctattttggcgttctggttgatcttgcaaacgcgagattttgcactcactaactacgacccagaggcaggaaggcgcttttacgagtttatgtggaagttgtattatgccgaatacttggtttatgttttcatttga

SEQ ID NO: 101 - DNA, 1197 bp, synthetic construct
  misc_feature 1..1197: note = CvPTS2 Gene
  source 1..1197: mol_type = other DNA
atggggttgtccttagtttgtacgttcagtttccaaactaactaccacacactactaaatccgcacaacaaaaacccgaaaaattcattgctctcctatcagcacccaaaaacacccattatcaagtctagttacgacaactttccatcaaaatactgtctaacgaaaaactttcatttgttgggcttaaattctcataatcgtatttccagtcagtcccgatcgatcagggccgggagtgaccaaattgaaggttctccacatcatgaaagtgacaattcaattgctacgaagattttaaactttgggcatacatgctggaagctacagcgaccgtatgtagttaaggggatgatcagcattgcctgcggcctattcggaagggaactcttcaataatagacatcttttttcttggggtttaatgtggaaagcttttttcgctttggttcctatccttagttttaacttcttcgccgctattatgaatcaaatttacgatgttgacatcgaccgtattaacaaacccgatctcccccttgtttcaggcgagatgtccattgaaacggcatggattttgtccatcattgttgcgcttactggcttgattgttaccattaagcttaaaagcgctcccttgttcgtttttatatacattttcggcatttttgccggattcgcatacagtgtcccgcctatacgttggaaacaatatccattcacgaacttcttgatcacgatctcatcacatgttggattggcctttacgtcctacagtgctaccacatctgcccttggattgcctttcgtttggaggcctgccttctcgtttatcattgcatttatgacagtgatgggaatgactatcgcatttgctaaagatatcagcgacatagagggcgatgcaaaatatggggtgagtactgttgcgacgaagttgggcgcccgaaatatgaccttcgttgtttccggcgttcttttacttaactatttagtatcgattagcatcgggatcatctggccacaggtgtttaaatcaaatattatgatcttgtcgcatgccatcctagctttctgtcttatatttcaaacaagagaattagccctagcgaactacgcctcagcaccaagtcgtcagttcttcgaatttatatggctactctactacgccgaatacttcgtctatgtcttcatttag

SEQ ID NO: 102 - DNA, 1635 bp, synthetic construct
  misc_feature 1..1635: note = CvCBD1 Gene
  source 1..1635: mol_type = other DNA
atgaagtgttctacgtttagtttttggtttgtttgtaaaattatattcttctttttttccttcaacattcagacatcaatcgccaacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacactga

SEQ ID NO: 103 - DNA, 1722 bp, synthetic construct
  misc_feature 1..1722: note = CBD1dNS1
  source 1..1722: mol_type = other DNA
atgttcttgaaacacatttttgttgctctcgcttttgccttgttagctgacgctaccccagcccagaagagatctcccggcttcgttgctttagactttgacatcgtcaaggttcaaaagaacgtgactgccaacgacgacgccgctgccattgttgccaagagacagaccaacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacactga

SEQ ID NO: 104 - DNA, 1632 bp, synthetic construct
  misc_feature 1..1632: note = CBD1dNS2
  source 1..1632: mol_type = other DNA
atgcaattgtcattgtcagttttgtcaacagttgcaacagcattgttgtcattgacaacagcagttgatgcaaagtcacataacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacactga

SEQ ID NO: 105 - DNA, 1827 bp, synthetic construct
  misc_feature 1..1827: note = CBD1dNV1
  source 1..1827: mol_type = other DNA
atgcaattgtccttgtcggttttatcaaccgttgccacggccttgttgtccctaaccaccgccgtcgatgctaagtcccacaacatcaagttgtccaagttgtccaacgaagaaacattggacgcctccacattccaagaatacacgagctccttggccaacaagtacatgaacttgttcaacgccgctcacggtaacccaaccagctttggcttgcaacacgtcttgtccaaccaagaagctgaagtcccattcgttaccccacaaaagggtggcaacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacactga

SEQ ID NO: 106 - DNA, 1572 bp, synthetic construct
  misc_feature 1..1572: note = CvCBD1dNP1
  source 1..1572: mol_type = other DNA
atgaacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacacggaagaagggcaaagttgtaa

SEQ ID NO: 107 - DNA, 1638 bp, synthetic construct
  misc_feature 1..1638: note = CvTHC1 Gene
  source 1..1638: mol_type = other DNA
atgaattgttcagcatttagtttttggtttgtttgtaagattattttcttctttttgtcatttaacattcaaatttcaattgcaaacccacaagaaaactttttgaagtgtttttcagaatacattccaaacaatccagctaacccaaagtttatttacacacaacatgatcaattgtacatgtcagttttgaactcaacaattcaaaacttgagatttacatcagataccacaccaaagccattggttattgttacaccatcaaacgtttcccatattcaagcatcaatcttgtgttcaaagaaggttggattgcaaattagaaccagatcaggaggacacgatgcagaaggaatgtcatacatttcacaagttccattcgttgttgttgatttgagaaacatgcactcaattaagattgatgttcattcacaaacagcatgggttgaagcaggagcaacattgggtgaagtttactactggattaacgaaaagaacgaaaacttcagttttccaggaggttactgtccaacagttggagttggaggacatttttcaggtggaggatacggagcattgatgagaaactacggattggcagcagataacattattgatgcacacttggttaacgttgatggaaaggttttggatagaaagtcaatgggagaagatttgttttgggcaattagaggaggtggtggagagaactttggaattattgcagcatggaagatcaagttggttgcagttccatcaaagtcaacaatcttttcagttaagaagaacatggaaattcatggtttggttaagttgtttaacaagtggcaaaacattgcatacaagtacgataaggatttggttttgatgacacattttattacaaagaacattacagataaccatggaaagaacaagacaacagttcacggatacttttcatcaatttttcacggaggagttgattcattggttgacttgatgaacaagtcatttccagaattgggaatcaagaagacagattgtaaggaattttcatggattgatacaacaattttctactcaggagttgttaactttaacacagcaaactttaagaaggaaattttgttggacagatcagcaggaaagaagaccgcattttccattaagttggattacgttaagaaaccaattccagaaacagcaatggttaagattttggaaaagttgtacgaagaagatgttggtgttggaatgtacgttttgtacccatacggaggaattatggaagaaatctcagaatcagcaattccatttccacatagagcaggtattatgtacgaattgtggtacacagcatcatgggaaaagcaagaagataatgaaaagcatattaactgggttagatcagtttacaactttacaacaccatacgtttcacaaaacccaagattggcatacttgaactacagagatttggatttgggaaagacaaacccagaatcaccaaacaactatacacaagctagaatttggggagaaaagtactttggtaagaacttcaacagattggttaaagttaagacaaaggcagatccaaataacttctttagaaacgaacaatcaattccaccattgccaccacatcatcattaa

SEQ ID NO: 108 - DNA, 1728 bp, synthetic construct
  misc_feature 1..1728: note = THC1dNS1 Gene
  source 1..1728: mol_type = other DNA
atgttcttgaaacacatttttgttgctctcgcttttgccttgttagctgacgctaccccagcccagaagagatctcccggcttcgttgctttagactttgacatcgtcaaggttcaaaagaacgtgactgccaacgacgacgccgctgccattgttgccaagagacagaccaacccacaagaaaactttttgaagtgtttttcagaatacattccaaacaatccagctaacccaaagtttatttacacacaacatgatcaattgtacatgtcagttttgaactcaacaattcaaaacttgagatttacatcagataccacaccaaagccattggttattgttacaccatcaaacgtttcccatattcaagcatcaatcttgtgttcaaagaaggttggattgcaaattagaaccagatcaggaggacacgatgcagaaggaatgtcatacatttcacaagttccattcgttgttgttgatttgagaaacatgcactcaattaagattgatgttcattcacaaacagcatgggttgaagcaggagcaacattgggtgaagtttactactggattaacgaaaagaacgaaaacttcagttttccaggaggttactgtccaacagttggagttggaggacatttttcaggtggaggatacggagcattgatgagaaactacggattggcagcagataacattattgatgcacacttggttaacgttgatggaaaggttttggatagaaagtcaatgggagaagatttgttttgggcaattagaggaggtggtggagagaactttggaattattgcagcatggaagatcaagttggttgcagttccatcaaagtcaacaatcttttcagttaagaagaacatggaaattcatggtttggttaagttgtttaacaagtggcaaaacattgcatacaagtacgataaggatttggttttgatgacacattttattacaaagaacattacagataaccatggaaagaacaagacaacagttcacggatacttttcatcaatttttcacggaggagttgattcattggttgacttgatgaacaagtcatttccagaattgggaatcaagaagacagattgtaaggaattttcatggattgatacaacaattttctactcaggagttgttaactttaacacagcaaactttaagaaggaaattttgttggacagatcagcaggaaagaagaccgcattttccattaagttggattacgttaagaaaccaattccagaaacagcaatggttaagattttggaaaagttgtacgaagaagatgttggtgttggaatgtacgttttgtacccatacggaggaattatggaagaaatctcagaatcagcaattccatttccacatagagcaggtattatgtacgaattgtggtacacagcatcatgggaaaagcaagaagataatgaaaagcatattaactgggttagatcagtttacaactttacaacaccatacgtttcacaaaacccaagattggcatacttgaactacagagatttggatttgggaaagacaaacccagaatcaccaaacaactatacacaagctagaatttggggagaaaagtactttggtaagaacttcaacagattggttaaagttaagacaaaggcagatccaaataacttctttagaaacgaacaatcaattccaccattgccaccacatcatcattaataa

SEQ ID NO: 109 - DNA, 1638 bp, synthetic construct
  misc_feature 1..1638: note = THC1dNS2
  source 1..1638: mol_type = other DNA
atgcaattgtcattgtcagttttgtcaacagttgcaacagcattgttgtcattgacaacagcagttgatgcaaagtcacataacccacaagaaaactttttgaagtgtttttcagaatacattccaaacaatccagctaacccaaagtttatttacacacaacatgatcaattgtacatgtcagttttgaactcaacaattcaaaacttgagatttacatcagataccacaccaaagccattggttattgttacaccatcaaacgtttcccatattcaagcatcaatcttgtgttcaaagaaggttggattgcaaattagaaccagatcaggaggacacgatgcagaaggaatgtcatacatttcacaagttccattcgttgttgttgatttgagaaacatgcactcaattaagattgatgttcattcacaaacagcatgggttgaagcaggagcaacattgggtgaagtttactactggattaacgaaaagaacgaaaacttcagttttccaggaggttactgtccaacagttggagttggaggacatttttcaggtggaggatacggagcattgatgagaaactacggattggcagcagataacattattgatgcacacttggttaacgttgatggaaaggttttggatagaaagtcaatgggagaagatttgttttgggcaattagaggaggtggtggagagaactttggaattattgcagcatggaagatcaagttggttgcagttccatcaaagtcaacaatcttttcagttaagaagaacatggaaattcatggtttggttaagttgtttaacaagtggcaaaacattgcatacaagtacgataaggatttggttttgatgacacattttattacaaagaacattacagataaccatggaaagaacaagacaacagttcacggatacttttcatcaatttttcacggaggagttgattcattggttgacttgatgaacaagtcatttccagaattgggaatcaagaagacagattgtaaggaattttcatggattgatacaacaattttctactcaggagttgttaactttaacacagcaaactttaagaaggaaattttgttggacagatcagcaggaaagaagaccgcattttccattaagttggattacgttaagaaaccaattccagaaacagcaatggttaagattttggaaaagttgtacgaagaagatgttggtgttggaatgtacgttttgtacccatacggaggaattatggaagaaatctcagaatcagcaattccatttccacatagagcaggtattatgtacgaattgtggtacacagcatcatgggaaaagcaagaagataatgaaaagcatattaactgggttagatcagtttacaactttacaacaccatacgtttcacaaaacccaagattggcatacttgaactacagagatttggatttgggaaagacaaacccagaatcaccaaacaactatacacaagctagaatttggggagaaaagtactttggtaagaacttcaacagattggttaaagttaagacaaaggcagatccaaataacttctttagaaacgaacaatcaattccaccattgccaccacatcatcattaataa

SEQ ID NO: 110 - DNA, 1833 bp, synthetic construct
  misc_feature 1..1833: note = THC1dNV1
  source 1..1833: mol_type = other DNA
atgcaattgtccttgtcggttttatcaaccgttgccacggccttgttgtccctaaccaccgccgtcgatgctaagtcccacaacatcaagttgtccaagttgtccaacgaagaaacattggacgcctccacattccaagaatacacgagctccttggccaacaagtacatgaacttgttcaacgccgctcacggtaacccaaccagctttggcttgcaacacgtcttgtccaaccaagaagctgaagtcccattcgttaccccacaaaagggtggcaacccacaagaaaactttttgaagtgtttttcagaatacattccaaacaatccagctaacccaaagtttatttacacacaacatgatcaattgtacatgtcagttttgaactcaacaattcaaaacttgagatttacatcagataccacaccaaagccattggttattgttacaccatcaaacgtttcccatattcaagcatcaatcttgtgttcaaagaaggttggattgcaaattagaaccagatcaggaggacacgatgcagaaggaatgtcatacatttcacaagttccattcgttgttgttgatttgagaaacatgcactcaattaagattgatgttcattcacaaacagcatgggttgaagcaggagcaacattgggtgaagtttactactggattaacgaaaagaacgaaaacttcagttttccaggaggttactgtccaacagttggagttggaggacatttttcaggtggaggatacggagcattgatgagaaactacggattggcagcagataacattattgatgcacacttggttaacgttgatggaaaggttttggatagaaagtcaatgggagaagatttgttttgggcaattagaggaggtggtggagagaactttggaattattgcagcatggaagatcaagttggttgcagttccatcaaagtcaacaatcttttcagttaagaagaacatggaaattcatggtttggttaagttgtttaacaagtggcaaaacattgcatacaagtacgataaggatttggttttgatgacacattttattacaaagaacattacagataaccatggaaagaacaagacaacagttcacggatacttttcatcaatttttcacggaggagttgattcattggttgacttgatgaacaagtcatttccagaattgggaatcaagaagacagattgtaaggaattttcatggattgatacaacaattttctactcaggagttgttaactttaacacagcaaactttaagaaggaaattttgttggacagatcagcaggaaagaagaccgcattttccattaagttggattacgttaagaaaccaattccagaaacagcaatggttaagattttggaaaagttgtacgaagaagatgttggtgttggaatgtacgttttgtacccatacggaggaattatggaagaaatctcagaatcagcaattccatttccacatagagcaggtattatgtacgaattgtggtacacagcatcatgggaaaagcaagaagataatgaaaagcatattaactgggttagatcagtttacaactttacaacaccatacgtttcacaaaacccaagattggcatacttgaactacagagatttggatttgggaaagacaaacccagaatcaccaaacaactatacacaagctagaatttggggagaaaagtactttggtaagaacttcaacagattggttaaagttaagacaaaggcagatccaaataacttctttagaaacgaacaatcaattccaccattgccaccacatcatcattaataa

SEQ ID NO: 111 - DNA, 1638 bp, synthetic construct
  misc_feature 1..1638: note = CvCBC1 Gene
  source 1..1638: mol_type = other DNA
atgaattgtagcactttctcattctggtttgtttgtaagattattttctttttcttgtcatttaacattcaaatttcaattgcaaacccacaagagaactttttgaagtgtttctcagaatacattccaaacaacccagctaacccaaagtttatttacacccaacacgatcaattgtacatgtcagttttgaactcaacaattcaaaacttgagatttacatcagatacaacaccaaagccattggttattgttacaccatcaaacgttagtcatattcaagcatcaatcttgtgttcaaagaaggttggattgcaaattagaactagatcaggaggacatgatgcagaaggattgtcatacatttcacaagttccatttgcaattgttgatttgagaaacatgcacacagttaaggttgatattcattcacaaacagcatgggttgaagcaggagcaacattgggtgaagtttactactggattaacgaaatgaacgaaaacttctcatttccaggaggatactgtccaacagttggtgttggaggacacttttcaggtggtggatacggagcattgatgagaaactacggattggcagcagataacattattgatgcacatttggttaacgttgatggaaaggttttggatagaaagtcaatgggagaagatttgttttgggcaattagaggaggtggaggagaaaactttggaatcattgcagcatgtaagatcaagttggttgttgttccatcaaaggcaacaatcttttcagttaagaagaacatggaaatccatggattggttaagttgtttaacaagtggcaaaacattgcatacaagtacgataaggatttgatgttgacaacacattttagaacaagaaacattacagataaccacggaaagaataagacaacagttcatggatacttttcatcaattttcttgggaggagttgattcattggttgacttgatgaacaagagttttccagaattgggaatcaagaagacagattgtaaggaattgtcatggatcgatacaaccattttctactcaggagttgttaactacaacacagctaactttaagaaggaaattttgttggacagatcagcaggtaaaaagacagcattttcaattaagttggattacgttaagaaattgattccagaaacagcaatggttaagattttggaaaagttgtacgaagaagaagttggagttggaatgtacgttttgtacccatacggaggaattatggatgaaatttcagaatcagcaattccatttccacatagagcaggtattatgtacgaattgtggtacacagcaacatgggaaaagcaagaagataacgaaaagcatattaactgggttagatcagtttacaactttacaaccccatacgtttcacaaaacccaagattggcatacttgaactacagagatttggatttgggaaagacaaacccagaatcaccaaataactacacacaagctagaatttggggagaaaagtactttggtaagaactttaacagattggtgaaggttaagacaaaggcagacccaaacaatttctttagaaacgaacaatcaattccaccattgccaccaagacatcattaa

SEQ ID NO: 112 - DNA, 2130 bp, synthetic construct
  misc_feature 1..2130: note = YlACO1P Gene
  source 1..2130: mol_type = other DNA
atgaccgaagtagttgacagagcctcatcccccgcatcccctggctcaactacggccgccgcagacggtgctaaggtggccgtcgagccccgagtagatgtggctgcgctgggagagcagctgctgggccgatgggctgatatccgtctccacgcccgggaccttgcgggacgagaggtagttcagaaggtggagggtctgactcatacagagcaccgctctcgcgtctttggccagctcaagtacttggtcgataacaacgcagttcaccgagcctttccttctcgactgggtggtagtgacgaccacggcggaaacatcgctggttttgaggagcttgtcacggcggacccctccctccagatcaaggccggcgtccagtggggactgttcggctccgctgttatgcacttgggaactagggagcaccacgacaagtggctcccaggcatcatgtctctggaaatccctggttgctttgccatgactgagactggccatggctccgatgtcgcttccattgctacaacggccacctatgatgaggaaacccaggagttcgttattgacaccccgttccgagccgcctggaaggactacattggaaacgccgctaacgacggtttggccgctgtcgtgtttgcccaactgattactcgaaaggttaaccatggagtgcacgccttctacgtcgatctgagagatcccgccaccggagactttctccctggtattggtggagaggacgacggtattaaggggggactaaacggaattgataacggacgtctccatttcaccaatgttcgcattccccgaaccaacctgcttaaccgttacggcgatgttgccgtcgatggcacctacagctcaaccatcgaatctccggggcgaagattctttacaatgctaggtacgctggtccagggccgagtcagcctggacggtgctgcagtggctgcatcgaaggttgctctgcaatccgccatccactacgccgctgagcgaagacagttcaacgccacttcgcccacagaggaggaggtgctcctggattaccagcgacaccagcggcgcctctttacccgactcgccaccacctacgccgcatcgttcgcccatgagcaactgctgcagaaattcgacgacgtgttctcgggtgctcatgatactgacgccgaccgtcaggaccttgagacactggctgctgctctgaagcccctttctacctggcatgctctcgataccctacaagagtgccgagaagcgtgtgggggtgcaggttttctgattgagaaccgattcgcttctctccgggccgatctcgacgtctacgtgaccttcgaaggagacaacaccgtgcttcttcagttggtggccaagaggctgctcgctgactatgctaaggagttccgaggtgccaacttcggcgtgctcgcgcggtacgtcgtggaccaggctgccggagtcgcgctacaccgaaccggactgcgacaggtcgctcagttcgtggccgacagtggatctgtccagaaatctgctcttgccctccgagacgaagaaggtcagcgaactctgctgaccgacagagtccagtccatggttgcagaggttggcgctgctctcaaaggcgcgggcaagctcccccagcaccaggcggcagcactgttcaatcagcatcaaaacgaactgatcgaggctgcccaggcccacgctgagcttttacagtgggaggcctttactgaggctttggccaaggtggacgacgctggcactaaggaagtgttgacccgattgcgtgacctttttggtctgtcccttatcgagaagcacctcagctggtatctgatgaacggtaggctctcgatgcagagaggccgaacggtcggcacttacattaatcgtcttctcgttaagatccgaccacacgcacttgatctggttgatgccttcggctacggagccgagcaccttcgggccgctatcgccaccggcgctgaggccacccgacaggacgaggcccgaacctacttcagacagcaacgagcctccggtagcgcccctgctgacgagaagacactcctcgctatcaaggccggcaagtctcggggacgacgagccaaactgtaa

SEQ ID NO: 113 - DNA, 2163 bp, synthetic construct
  misc_feature 1..2163: note = YlHXS1 Gene
  source 1..2163: mol_type = other DNA
atgggaaagaattacaaaagtctagattctgtcgttgccagtgacttcatcgcgttaggcattacatccgaggtcgctgagactctgcacggacggcttgccgagattgtgtgcaactacggagccgctacccctcagacttggattaacatcgccaaccacattctgtcgccggacctccccttctctttgcaccagatgttattctacggatgctacaaggattttggccctgcacctcctgcctggattccggaccccgaaaaagtcaagtccaccaacctaggtgccctgctggaaaagcgaggaaaggagttccttggtgtcaagtacaaggaccccatttcttctttttctcatttccaggaattttcggtgcgtaatcctgaggtgtattggcgaactgtgctcatggacgagatgaaaatctccttcagcaaggacccagagtgtatcctgcgacgagacgacattaacaacccaggaggctcggagtggcttcccggcggatacctaaactcagctaagaattgtctcaacgtgaactctaacaagaagttgaacgacaccatgatcgtgtggcgtgacgaaggcaacgacgacctgcccctgaacaagttgactctggaccagctgcgaaagagggtctggttggttggctacgccctcgaagagatgggcttagagaagggttgcgctattgctattgatatgcccatgcacgtcgatgctgtagtgatctaccttgccattgtgttagccggttacgtggtcgtatcgattgccgattcgttctccgctccggagatttccacccgactcagacttagcaaggccaaggcaatctttactcaagatcacatcatccgaggtaagaagagaatccctctctattctcgcgtggttgaggccaagtccccaatggctatcgtcataccttgcagcggatcaaacatcggggctgagctacgggacggtgatatctcctgggattacttcctggagcgagccaaagagttcaagaactgcgagtttacagcgcgtgagcagcccgtcgatgcctacacgaacattctattctcatcgggcacaacgggagagcccaaggccatcccctggacccaagctacccccttgaaagctgccgctgatggttggtcccatctcgacatcagaaaaggcgatgtgatcgtttggcccactaacctgggctggatgatgggtccttggctggtatatgccagcctactgaacggcgcttcaatcgcactgtacaacggatctccactcgtcagcggctttgccaagtttgttcaagacgccaaagtcaccatgctgggtgttgttccttcaatcgtgcgaagttggaagagtaccaactgtgtctctggatacgactggagcaccattcgatgcttcagttcctccggcgaggcttccaacgttgatgagtacctctggcttatgggtcgtgcgaattacaagcctgtgatcgagatgtgtggtggaacagaaattggtggtgctttttcggccgggtcctttcttcaggctcagtctctctcctctttctcttcccagtgtatgggatgcaccctgtatattctcgacaagaacggttaccccatgccgaagaataaacccggtattggggagcttgctcttggccccgtcatgtttggtgcatcgaagaccctcctgaacggaaaccatcacgacgtctacttcaagggcatgcccacacttaacggcgaagttcttcggcgacatggagacattttcgaacttacatcgaacggatactaccacgcccacgggcgagcagatgatacgatgaacatcgggggcatcaagatatcttctatcgagattgaaagagtgtgtaacgaggtagacgaccgcgtcttcgagactactgcgatcggcgtcccccccctgggcggtggcccggagcagctagtcattttttttgtgctcaaggactctaacgacacgaccatcgacctcaatcagctgcgactctccttcaaccttggattgcagaagaagctgaaccctctcttcaaggtcactcgggttgttcccttgtcctctcttcctcgaaccgccaccaacaagattatgcgacgagtgctccgacagcagttctcccacttcgagtaa

SEQ ID NO: 114 - DNA, 1158 bp, synthetic construct
  misc_feature 1..1158: note = YlTKS1 Gene
  source 1..1158: mol_type = other DNA
atgaatcatctaagagccgaaggaccagcaagtgtactcgctattggtactgctaaccccgagaacattcttattcaggatgagttcccggactactatttcagggttaccaagagcgagcatatgacccagcttaaagagaagttccgcaagatatgcgacaagtccatgatccgaaagcggaactgctttctcaatgaggagcacttgaagcaaaacccccgactggttgagcacgagatgcagaccttggacgcgcgacaagacatgctcgtcgtcgaggtgcccaaactcggtaaggatgcttgcgctaaggccattaaggagtggggtcagcccaagtcgaagattacccacctaatcttcacctccgcaagcactacagacatgcccggtgcagactaccactgtgccaagctgctcggactgtcaccgtcggtcaagcgagtgatgatgtaccagctcggctgttacgggggtggaaccgttctccgtatcgccaaggatatcgctgagaacaacaaaggagctcgtgtcctggctgtgtgttgcgacatcatggcctgcttgttcagaggccctagtgattccgatctggaattacttgtcggtcaggccatctttggagatggcgccgccgctgtcatcgtgggtgccgaacccgacgagtctgttggagaaagacccatctttgagcttgtctccacgggccagaccatcctccctaacagcgagggcacaattggaggccatattcgagaggccggtctgatttttgacctgcataaggacgtgcctatgctgatttcgaacaacatcgagaagtgtctcatcgaggccttcactcccatcggcatttcggactggaactcaatcttctggatcacccacccaggaggcaaggcgattctggataaagttgaggaaaagctcgaccttaagaaggagaagtttgtggattctcgacacgtcctgtctgaacacggtaacatgtcttcctctactgtcctgttcgtaatggacgagcttcgaaagcgatctctggaggaaggaaagtccacgaccggcgacggttttgagtggggcgtgctgttcgggttcggtcctggcctcactgtggagcgagttgttgtccggtccgtgcctattaagtactaa

SEQ ID NO: 115 - DNA, 1176 bp, synthetic construct
  misc_feature 1..1176: note = YlTKS1P Gene
  source 1..1176: mol_type = other DNA
atgaatcatctaagagccgaaggaccagcaagtgtactcgctattggtactgctaaccccgagaacattcttattcaggatgagttcccggactactatttcagggttaccaagagcgagcatatgacccagcttaaagagaagttccgcaagatatgcgacaagtccatgatccgaaagcggaactgctttctcaatgaggagcacttgaagcaaaacccccgactggttgagcacgagatgcagaccttggacgcgcgacaagacatgctcgtcgtcgaggtgcccaaactcggtaaggatgcttgcgctaaggccattaaggagtggggtcagcccaagtcgaagattacccacctaatcttcacctccgcaagcactacagacatgcccggtgcagactaccactgtgccaagctgctcggactgtcaccgtcggtcaagcgagtgatgatgtaccagctcggctgttacgggggtggaaccgttctccgtatcgccaaggatatcgctgagaacaacaaaggagctcgtgtcctggctgtgtgttgcgacatcatggcctgcttgttcagaggccctagtgattccgatctggaattacttgtcggtcaggccatctttggagatggcgccgccgctgtcatcgtgggtgccgaacccgacgagtctgttggagaaagacccatctttgagcttgtctccacgggccagaccatcctccctaacagcgagggcacaattggaggccatattcgagaggccggtctgatttttgacctgcataaggacgtgcctatgctgatttcgaacaacatcgagaagtgtctcatcgaggccttcactcccatcggcatttcggactggaactcaatcttctggatcacccacccaggaggcaaggcgattctggataaagttgaggaaaagctcgaccttaagaaggagaagtttgtggattctcgacacgtcctgtctgaacacggtaacatgtcttcctctactgtcctgttcgtaatggacgagcttcgaaagcgatctctggaggaaggaaagtccacgaccggcgacggttttgagtggggcgtgctgttcgggttcggtcctggcctcactgtggagcgagttgttgtccggtccgtgcctattaagtacggaagaagggcaaagttgtaa

SEQ ID NO: 116 - DNA, 318 bp, synthetic construct
  misc_feature 1..318: note = YlOAC1 Gene
  source 1..318: mol_type = other DNA
atggccgtcaaacaccttattgtcctcaagttcaaagatgagatcactgaagcccagaaggaggagtttttcaagacctacgtcaatttggtcaacatcattccagcaatgaaggatgtgtactggggcaaggacgtgacccagaagaacaaggaagagggttatacccatatcgttgaggttacgttcgagtctgtggagacaatccaagactacatcattcaccccgctcacgtgggctttggagacgtttacagatccttctgggagaagctcctgatttttgactacactcctcgaaagctgaagcccaagtaa

SEQ ID NO: 117 - DNA, 336 bp, synthetic construct
  misc_feature 1..336: note = YlOAC1P Gene
  source 1..336: mol_type = other DNA
atggccgtcaaacaccttattgtcctcaagttcaaagatgagatcactgaagcccagaaggaggagtttttcaagacctacgtcaatttggtcaacatcattccagcaatgaaggatgtgtactggggcaaggacgtgacccagaagaacaaggaagagggttatacccatatcgttgaggttacgttcgagtctgtggagacaatccaagactacatcattcaccccgctcacgtgggctttggagacgtttacagatccttctgggagaagctcctgatttttgactacactcctcgaaagctgaagcccaagggaagaagggcaaagttgtaa

SEQ ID NO: 118 - DNA, 1197 bp, synthetic construct
  misc_feature 1..1197: note = YlPTS2 Gene
  source 1..1197: mol_type = other DNA
atgggcctctctctagtatgtaccttctctttccagaccaactatcacactctactgaacccccataacaagaaccctaaaaattctcttctcagttaccagcaccccaagacgcctatcattaagtcctcctacgacaactttccctctaagtactgcctgaccaaaaacttccatctcctgggactgaactctcataacagaattagtagccagtcccgatctatccgagctggctctgaccagattgagggctcccctcaccatgaatccgacaacagcatcgctaccaagattttgaattttggtcacacatgctggaagctccagcgaccgtacgtcgtgaagggtatgatctcgattgcctgtggactgttcggacgtgagctttttaataatcgacacttgttttcatggggcctcatgtggaaggcttttttcgccctcgtgcccattctgtctttcaacttctttgccgctattatgaaccaaatctacgacgttgatattgataggatcaacaagcctgacctgccgctcgtctcgggggagatgtctatcgagacagcgtggattctttcgattatcgtcgcgctgactggccttatcgttaccataaagttgaagtctgcacccctcttcgtgtttatctacattttcggtatttttgctggattcgcgtactccgttccccctatcagatggaagcagtacccctttactaactttctgattactatcagcagccacgtcggtttagcctttacctcatattcggccaccaccagtgcactgggcctccccttcgtctggcgacctgcattttcattcatcatcgccttcatgactgtgatgggtatgaccatcgctttcgctaaggacatctccgacatcgagggtgatgctaaatatggagtgtccaccgtggccactaagctgggagcccggaacatgacgttcgtcgtctctggtgttctgctccttaactacttggtttcgatctccattggcattatctggccacaagtcttcaagtccaacattatgattctgtcccacgccattcttgccttttgcctgatcttccagacacgcgaactcgctctcgctaactacgcctccgccccatcgcgacagttcttcgagttcatctggctgctttactacgccgagtacttcgtttacgtgttcatctaa

SEQ ID NO: 119 - DNA, 1635 bp, synthetic construct
  misc_feature 1..1635: note = YlCBD1 Gene
  source 1..1635: mol_type = other DNA
atgaagtgttcgacgttttctttttggtttgtttgtaaaatcattttctttttcttttctttcaacatccaaacgtcgatcgcaaaccctagagagaactttcttaagtgcttctcgcagtacatccctaataacgctaccaaccttaagctggtgtacacccagaacaaccctctttacatgtctgttctaaacagcaccatccacaatcttagattcacatcagacaccactcccaagccgctcgtcatcgtgaccccgagtcatgtgtcccatatccaaggcactatcctgtgctctaaaaaggtcggtctgcagattcggactcgctccggtggacatgattcggagggcatgtcctacattagccaggtcccctttgtgatcgtggacctgaggaacatgcggtctattaagattgatgtgcactcacagaccgcttgggtcgaggctggtgcgacattgggtgaggtgtactactgggtgaacgagaagaacgagaacctgagcctcgccgctggctactgtcccaccgtttgtgccggtggacacttcggcggaggcggatacggtccacttatgcgaaactacgggctcgcagctgataatatcatcgacgcacaccttgttaacgttcacggcaaggtgctggaccgaaaaagcatgggtgaggacctattttgggccttgcgaggcggtggtgccgaatccttcggaattatcgtggcctggaagatccgactggtcgctgtgccaaagtccactatgttctccgtcaagaaaattatggagatccacgaactcgtaaagctcgtcaataagtggcagaacatcgcctacaagtatgacaaggatctgctgctcatgactcacttcatcacgcgaaacattacagacaaccagggaaagaacaagaccgctatccatacctacttctcctctgtcttccttgggggtgtcgattccctcgttgatctcatgaacaaatcttttccagagctcggaatcaagaagaccgactgccgacagctctcttggatcgacaccattattttctactcaggagtcgtaaactacgatactgacaactttaacaaggagattctgttagatcgatcggccggccagaacggtgccttcaagatcaagctcgactatgtcaaaaagcccattcctgaatccgtcttcgttcaaattcttgaaaagttgtacgaggaggatatcggcgccggaatgtacgcgctgtacccctacggtggcattatggacgagatttctgaaagtgctattcccttcccccaccgtgctggcattctgtatgagctgtggtacatttgctcctgggaaaagcaggaggacaacgagaagcacttgaactggatacgaaacatttacaatttcatgaccccctatgtttcgaagaaccctcgactggcctacctgaattaccgcgacctcgacatcggaattaacgaccctaagaaccccaataactatactcaggccagaatctggggcgagaagtacttcggcaagaactttgaccgtctggttaaggtcaagaccctcgtggaccctaacaacttcttccgaaacgagcagtctatcccccctctgccccgacaccggcattaa

SEQ ID NO: 120 - DNA, 1638 bp, synthetic construct
  misc_feature 1..1638: note = YlTHC1 Gene
  source 1..1638: mol_type = other DNA
atgaattgttcagctttctctttttggtttgtctgtaagatcattttcttctttctatcctttcacatccaaatttccatagccaaccctcgtgagaacttccttaagtgcttttccaagcacattccaaataacgtcgccaatcccaagctggtgtacacgcagcatgaccagctctacatgtcgatcctcaattccaccattcaaaaccttagattcattagtgacaccactcccaagcccctagtcattgtcaccccttcgaacaactcgcatattcaggcaactattctctgctccaagaaggttggtttacagatccgaacccggtcaggtggtcacgacgctgagggcatgtcttacatttcccaggtcccctttgtggtggtcgatctgcgcaacatgcactccattaaaatcgacgtccactcgcagactgcctgggtcgaggctggagccacccttggcgaggtctactactggattaacgagaagaacgaaaacctgtcgttccctggcggctactgtccgactgttggagtcggcggacacttttctggtggcggatatggtgctctcatgcgaaactacggactggcggcagacaacatcatcgatgcccaccttgtgaacgttgacggtaaggtactggaccgaaagtctatgggcgaggacttgttttgggccatccgaggtggaggtggtgagaacttcgggatcatcgccgcctggaagatcaagctggtggatgtgcccagtaagtctaccatttttagcgtgaagaagaacatggagatccacgggctggtgaagctgttcaacaagtggcagaatattgcgtacaaatacgacaaggacctggtgcttatgacccatttcatcaccaagaacatcacggataaccacggtaaaaacaagactactgttcacggttacttctcttcaattttccatggtggtgtggattccctcgttgatttgatgaacaagtccttcccagagctgggcattaagaagacagactgcaaggaatttagctggattgataccaccatcttctactctggagttgtcaacttcaacaccgcaaacttcaagaaggaaatcctcttggaccgatctgccggcaagaagacagctttttcgattaaactggattacgtgaagaagcccatccctgagacagctatggtcaagatccttgaaaaactttatgaggaggacgtcggagccggaatgtacgttctctatccttacggcggcatcatggaggaaatttctgagtctgctatccccttcccccatcgagccggaatcatgtacgagctgtggtacaccgctagttgggagaagcaggaggataacgagaaacatatcaattgggtccgtagcgtatacaatttcacgacaccctacgtgtcccagaaccctcgactcgcttacctgaactatagggacctggacctcggcaagactaaccacgctagcccgaacaactacacccaggccagaatttggggcgaaaagtacttcggaaagaacttcaaccgactcgttaaggttaagaccaaagttgaccccaacaactttttccggaacgagcagtccatccctccactccctccccaccatcactga

SEQ ID NO: 121 - DNA, 26 bp, synthetic construct
  misc_feature 1..26: note = CvACT1
  source 1..26: mol_type = other DNA
cccaattcctgtggtgggttgattcg

SEQ ID NO: 122 - DNA, 29 bp, synthetic construct
  misc_feature 1..29: note = CvACT1
  source 1..29: mol_type = other DNA
ctctcaattcgttgtagaaggtgtggtgc

SEQ ID NO: 123 - DNA, 29 bp, synthetic construct
  misc_feature 1..29: note = CvHXS1
  source 1..29: mol_type = other DNA
ctttggactcagttgtggctagtgacttc

SEQ ID NO: 124 - DNA, 34 bp, synthetic construct
  misc_feature 1..34: note = CvHXS1
  source 1..34: mol_type = other DNA
gatcaagagtcaatttattcaatgggagatcgtc

SEQ ID NO: 125 - DNA, 23 bp, synthetic construct
  misc_feature 1..23: note = CvTKS1
  source 1..23: mol_type = other DNA
gagcagaaggacccgcatcagtg

SEQ ID NO: 126 - DNA, 28 bp, synthetic construct
  misc_feature 1..28: note = CvTKS1
  source 1..28: mol_type = other DNA
gtcaccaaaaattgcctgtccgacaagc

SEQ ID NO: 127 - DNA, 31 bp, synthetic construct
  misc_feature 1..31: note = CvOAC1
  source 1..31: mol_type = other DNA
cacgacataatggcagtcaaacacctaatag

SEQ ID NO: 128 - DNA, 31 bp, synthetic construct
  misc_feature 1..31: note = CvOAC1
  source 1..31: mol_type = other DNA
ctagttttgtgttcggagtatgcatacaacg

SEQ ID NO: 129 - DNA, 25 bp, synthetic construct
  misc_feature 1..25: note = CvPTS1
  source 1..25: mol_type = other DNA
ctaccacaccctcctaaaccctcac

SEQ ID NO: 130 - DNA, 31 bp, synthetic construct
  misc_feature 1..31: note = CvPTS1
  source 1..31: mol_type = other DNA
cgaagcagtacccgaatatatataatggacc

SEQ ID NO: 131 - DNA, 29 bp, synthetic construct
  misc_feature 1..29: note = CvCBD1
  source 1..29: mol_type = other DNA
ccttcaacattcagacatcaatcgccaac

SEQ ID NO: 132 - DNA, 22 bp, synthetic construct
  misc_feature 1..22: note = CvCBD1
  source 1..22: mol_type = other DNA
cacccaaggttgcccctgcttc

SEQ ID NO: 133 - DNA, 24 bp, synthetic construct
  misc_feature 1..24: note = CvACO1
  source 1..24: mol_type = other DNA
gagcaagttccccagcaagtccag

SEQ ID NO: 134 - DNA, 26 bp, synthetic construct
  misc_feature 1..26: note = CvACO1
  source 1..26: mol_type = other DNA
catgatgttcacgggttcccaagtgc

SEQ ID NO: 135 - DNA, 25 bp, synthetic construct
  misc_feature 1..25: note = CvPTS2
  source 1..25: mol_type = other DNA
catacatgctggaagctacagcgac

SEQ ID NO: 136 - DNA, 27 bp, synthetic construct
  misc_feature 1..27: note = CvPTS2
  source 1..27: mol_type = other DNA
cagtactcaccccatattttgcatcgc

SEQ ID NO: 137 - DNA, 36 bp, synthetic construct
  misc_feature 1..36: note = CvTHC1
  source 1..36: mol_type = other DNA
gttaagttgtttaacaagtggcaaaacattgcatac

SEQ ID NO: 138 - DNA, 34 bp, synthetic construct
  misc_feature 1..34: note = CvTHC1
  source 1..34: mol_type = other DNA
ccaaaatcttaaccattgctgtttctggaattgg

SEQ ID NO: 139 - DNA, 29 bp, synthetic construct
  misc_feature 1..29: note = Candida viswanathii
  source 1..29: mol_type = other DNA
gatttgttttgggcaattagaggaggtgg

SEQ ID NO: 140 - DNA, 32 bp, synthetic construct
  misc_feature 1..32: note = Candida viswanathii
  source 1..32: mol_type = other DNA
caactcctgagtagaaaatggttgtatcgatc

SEQ ID NO: 141 - DNA, 29 bp, synthetic construct
  misc_feature 1..29: note = Yarrowia lipolytica
  source 1..29: mol_type = other DNA
ttgttaccaactgggatgacatggagaag

SEQ ID NO: 142 - DNA, 28 bp, synthetic construct
  misc_feature 1..28: note = Yarrowia lipolytica
  source 1..28: mol_type = other DNA
ctgatggacgtgtttttcgacatgaacc

SEQ ID NO: 143 - DNA, 34 bp, synthetic construct
  misc_feature 1..34: note = Yarrowia lipolytica
  source 1..34: mol_type = other DNA
gaaaggaacatagtcatttccaaacttgaaagtc

SEQ ID NO: 144 - DNA, 24 bp, synthetic construct
  misc_feature 1..24: note = Yarrowia lipolytica
  source 1..24: mol_type = other DNA
cagacggagtactttcgctcgagg

SEQ ID NO: 145 - DNA, 29 bp, synthetic construct
  misc_feature 1..29: note = Yarrowia lipolytica
  source 1..29: mol_type = other DNA
cccaaatttagctgcatcattcatcaacc

SEQ ID NO: 146 - DNA, 27 bp, synthetic construct
  misc_feature 1..27: note = Yarrowia lipolytica
  source 1..27: mol_type = other DNA
ccgtgcttaagagcaagttccttgagg

SEQ ID NO: 147 - DNA, 23 bp, synthetic construct
  misc_feature 1..23: note = Yarrowia lipolytica
  source 1..23: mol_type = other DNA
ctacgacatgcccaaggagcagc

SEQ ID NO: 148 - DNA, 26 bp, synthetic construct
  misc_feature 1..26: note = Yarrowia lipolytica
  source 1..26: mol_type = other DNA
ggattcgcacattggtgaactggatc

SEQ ID NO: 149 - DNA, 24 bp, synthetic construct
  misc_feature 1..24: note = Yarrowia lipolytica
  source 1..24: mol_type = other DNA
ccaagcgacgacaagctgttgagc

SEQ ID NO: 150 - DNA, 21 bp, synthetic construct
  misc_feature 1..21: note = Yarrowia lipolytica
  source 1..21: mol_type = other DNA
cgtgtgggtagcagagtgggc

SEQ ID NO: 151 - DNA, 26 bp, synthetic construct
  misc_feature 1..26: note = Yarrowia lipolytica
  source 1..26: mol_type = other DNA
ctttgccatgactgagactggccatg

SEQ ID NO: 152 - DNA, 18 bp, synthetic construct
  misc_feature 1..18: note = Yarrowia lipolytica
  source 1..18: mol_type = other DNA
gaggcgccgctggtgtcg

SEQ ID NO: 153 - DNA, 27 bp, synthetic construct
  misc_feature 1..27: note = Yarrowia lipolytica
  source 1..27: mol_type = other DNA
tccgaaagcggaactgctttctcaatg

SEQ ID NO: 154 - DNA, 23 bp, synthetic construct
  misc_feature 1..23: note = Yarrowia lipolytica
  source 1..23: mol_type = other DNA
cagaccggcctctcgaatatggc

SEQ ID NO: 155 - DNA, 26 bp, synthetic construct
  misc_feature 1..26: note = Yarrowia lipolytica
  source 1..26: mol_type = other DNA
ctagactacacgggcaaccttaaccc

SEQ ID NO: 156 - DNA, 25 bp, synthetic construct
  misc_feature 1..25: note = Yarrowia lipolytica
  source 1..25: mol_type = other DNA
cttgggcttcagctttcgaggagtg

SEQ ID NO: 157 - DNA, 4435 bp, synthetic construct
  misc_feature 1..4435: note = pLD1
  source 1..4435: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataagaagagcccgggtctagatgtgtgctcttccgagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 158 - DNA, 5564 bp, synthetic construct
  misc_feature 1..5564: note = pLD10
  source 1..5564: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgaatcatttaagagcagaaggacccgcatcagtgttagcgataggtacagctaacccagagaatatcttaatccaagatgaatttcctgactactatttccgtgttactaaatcggaacatatgactcaacttaaagagaagttccggaaaatctgcgataaatccatgatccgaaagagaaactgtttccttaacgaagaacatctcaagcaaaacccgaggttggtagagcacgaaatgcagaccttggatgctaggcaggacatgttggtggtcgaagtgccaaaactcggcaaggacgcgtgcgctaaggcaatcaaggagtggggtcaaccgaagtctaaaatcacgcatctaatatttacatctgcactgacaaccgacatgccgggtgccgattatcactgcgccaagctacttggattgagtccactggttaagagagttatgatgtatcaattggggtgttacggagggggcacagtcctcagaattgctaaggatattgcggaaaataacaagggcgcgagggtccttgctgtatgttgtgatattatggcctgtttgtttcgcgggccctcggattcagatttggaattgcttgtcggacaggcaatttttggtgacggggccgcagcagtcatagtgggagccgaaccagacgaaagcgtgggtgaaagaccaatctttgagttggttctgaccggacaaacgatcttacctaactcggaaggtacgattggaggacatattagagaagccggcctaattttcgatcttcacaaagacgttccaatgttaatctccaataacatagaaaagtgcttgatagaagcatttactcccattggtattagtgactggaacagcattttctggatcacccaccctggaggaaaagctatactcgataaggttgaagagaaactcgacttgaaaaaggagaaattcgttgactcacgacatgtgttatcagagcacgggaatatgagttcatccacagtcttgttcgtaatggatgaattgcgaaaacgctctcttgaggagggaaagagcacaaccggtgacgggtttgagtggggcgtgctattcggttttggcccaggtttgactgtcgagcgggttgttgttcgtagtgtaccaattaagtactgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 159 - DNA, 4724 bp, synthetic construct
  misc_feature 1..4724: note = pLD12
  source 1..4724: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatggcagtcaaacacctaatagttctcaaatttaaagacgagattactgaagctcagaaggaagagttctttaagacatatgttaacttagtcaacatcatccccgcgatgaaggacgtctactggggcaaggatgtgacgcaaaaaaataaggaagaaggatacacacatatcgttgaggtgacctttgagagtgtggaaactattcaagattatattattcacccagcccatgtagggttcggtgacgtttatcgatcattctgggaaaagttgcttatatttgattacaccccaagaaaattgaagcctaagtgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 160 - DNA, 4742 bp, synthetic construct
  misc_feature 1..4742: note = pLD14
  source 1..4742: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatggcagtcaaacacctaatagttctcaaatttaaagacgagattactgaagctcagaaggaagagttctttaagacatatgttaacttagtcaacatcatccccgcgatgaaggacgtctactggggcaaggatgtgacgcaaaaaaataaggaagaaggatacacacatatcgttgaggtgacctttgagagtgtggaaactattcaagattatattattcacccagcccatgtagggttcggtgacgtttatcgatcattctgggaaaagttgcttatatttgattacaccccaagaaaattgaagcctaagggaagacgagctaagttgtgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 161 - DNA, 6569 bp, synthetic construct
  misc_feature 1..6569: note = pLD16
  source 1..6569: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgggaaaaaattataaatctttggactcagttgtggctagtgacttcattgcacttgggatcacatcagaagttgctgagacattgcacggacgcttggcagagatagtttgcaactacggcgccgcaacacctcagacctggattaacatcgcaaaccatattctaagtccagatcttccatttagtctccatcagatgttgttctacggttgttataaggactttggtccagcacccccagcttggataccagaccccgaaaaagtaaagtccacgaacttaggtgccttgttagaaaagcggggaaaggagtttctaggcgttaagtataaggacccaataagtctgttttctcacttccaggagtttagcgttcgaaatccggaagtctactggcggacggtacttatggatgaaatgaagatactgttcagcaaagatcccgaatgtatcctcagacgcgacgacattaacaacccagggggctctgagtggctaccaggtggatatctcaacctggccaagaactgtttgaatgtaaatagtaacaaaaaacttaacgacactatgatagtgtggagagatgaaggaaatgacgatctcccattgaataaattgactcttgatcaattacgaaaacgagtctggttggttggatacgccctagaagagatgggccttgagaagggatgtgcgattgcaattgacatgcccatgcacgtagatgcggttgtgatctatttagctatcgtcttggcaggctacgtcgttgtctccattgcagattcattctcagcaccggaaatttccacaagattgcgtctatcaaaggctaaggctatttttacacaagatcatatcatccgagggaaaaagcgtatacctttgtacctgcgtgtcgtcgaggccaagtctccgatggcaatagttatcccgtgttcgggttcaaatattggtgcggaattgcgggatggtgatattctgtgggattacttcttagaacgcgcaaaggaatttaagaactgcgaatttacagcccgtgaacagccagtggacgcgtacacaaatattttgttctcatcgggaaccaccggagagccaaaggcgataccatggactcaagctacgcctctcaaggcggctgctgatggttggtcacacttggacattagaaagggtgacgtaattgtatggcctaccaatttggggtggatgatggggccttggttggtctatgcttcactccttaacggggcaagcatcgcattgtataacggatctccactagtgtccggctttgccaaattcgttcaagatgcgaaagttactatgctaggagttgtcccctccatcgtacgaagctggaaaagcactaattgcgttagtgggtacgattggtctacaatcagatgcttctcctcatcgggtgaggcatcgaatgtcgatgaatacttatggctaatgggaagggctaactacaaaccggtcatcgaaatgtgcggtggcacagagatcgggggtgccttcagcgccggttcgtttttacaagcccaatctttgagtagcttctcatcccaatgtatgggatgcaccttgtacattctcgacaagaatggctacccgatgccaaagaacaagccgggtataggtgaattggccttgggacccgtgatgttcggtgcttccaagactttacttaacggaaaccatcatgacgtttatttcaaaggcatgcccaccttgaacggagaagtcttgaggagacacggagatatcttcgaactcacttcgaacggctattatcacgctcatggtagagcagatgacacgatgaatatcggggggattaaaatttcctcaatcgagattgaaagggtgtgtaatgaagttgacgatagagtgtttgagactacggccattggagtgcctccattgggcggaggtccagagcagctcgttatcttttttgttcttaaggacagcaatgatacgaccatcgacctaaaccaattgcgacttagttttaatcttgggttacaaaagaaattgaacccactttttaaggtgacgagggttgtgccactttcgctgttgcctaggacagccaccaacaaaataatgagaagagtgcttagacagcaatttagtcatttcgagtgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 162 - DNA, 6041 bp, synthetic construct
  misc_feature 1..6041: note = pLD20
  source 1..6041: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgaagtgttctacgtttagtttttggtttgtttgtaaaattatattcttctttttttccttcaacattcagacatcaatcgccaacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacactgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 163 - DNA, 6536 bp, synthetic construct
  misc_feature 1..6536: note = pLD22
  source 1..6536: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgacagaagtagtagatagagcaagttccccagcaagtccaggatctacgaccgccgccgcagacggtgctaaggtggcggtggagccacgcgtagatgtagcggcccttggcgagcagttgctagggcgatgggctgacatcagattgcacgcacgagacttagcaggccgcgaagtggtccaaaaggttgaaggacttacgcacactgagcatcggagtagagtctttggacagttgaagtacttggtagacaacaatgctgttcacagagcttttccctccaggctaggtggatcagatgaccatggcggtaatatagctggattcgaggaattagttactgctgatccatcattgcaaataaaggccggcgttcagtggggtttgtttggttctgcagtgatgcacttgggaacccgtgaacatcatgacaagtggttgccaggtattatgtcgttagaaataccggggtgtttcgcgatgacagaaaccgggcacggtagcgacgtggcctctattgctacaacagcaacttatgatgaggaaacccaagagtttgttattgataccccgttcagagccgcttggaaagattatatcggtaatgcagcgaacgatggtttggcggcagttgttttcgcacaattaatcacgaggaaagtgaaccatggtgtacacgccttttacgtggatctcagagatcctgcgactggagacttcctacccggaataggaggagaggacgatggaatcaaggggggattgaatggcattgacaacggtagactacattttacgaacgtacgcattcctagaactaatcttcttaacagatatggcgatgtggcggtcgacggcacatacctgtcgaccatcgaatcaccagggcgccggttctttacgatgcttggtactctagtccagggtagagttagtctcgatggagcagctgtcgctgcactgaaggtcgcattgcaaagtgcaattcactacgctgcggagaggagacaatttaatgcgacttcacctactgaagaagaggtccttcttgattatcagaggcatcaaaggagactctttacacgacttgcaacgacgtacgccgcatctttcgcccacgagcagctattgcaaaagttcgatgatgtcttttcaggggcacatgataccgacgccgaccggcaggacttggaaaccctagccgccgctttgaagccattgagcacatggcatgcacttgacacgttacaagaatgcagagaggcctgtgggggggccggatttttgatagaaaaccgtttcgcgagcttgcgtgctgacttggacgtttacgtcacattcgagggtgataacacagttttattgcaattggttgctaaacggctcttggcagactacgcaaaagagttcagaggggccaacttcggcgttcttgccaggtatgtggttgaccaagccgcgggagtggcgctccaccgaacaggactaaggcaagtcgctcaatttgttgcagacagcgggtccgttcagaagtcggctcttgcgcttcgcgatgaagagggtcaacgaacattgttaacggacagagtacagagcatggttgccgaagtgggggctgccttgaaaggcgcaggcaaattaccccaacatcaagcagctgcattgttcaaccaacaccagaacgaacttattgaggctgcccaggcccatgcagaactcctccaatgggaggcatttacagaagctctcgctaaagtcgacgatgctggtacaaaggaagtgcttactcgattgcgagatctctttggtttgtccttgattgaaaaacacttgctgtggtatcttatgaatggacgtttgtccatgcaaagaggcaggacagttggaacttacattaatcgtttacttgtcaagatccgtccacacgcactagacttggttgatgccttcggttacggcgcggagcatttgcgtgctgctatcgccaccggagcggaagcaacccgacaggatgaagcccgaacgtattttagacaacaacgggcatcgggactggccccggccgatgaaaagaccttactcgctatcaaagctggtaaatcaagagggcgaagggcaaagctatgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 164 - DNA, 5582 bp, synthetic construct
  misc_feature 1..5582: note = pLD24
  source 1..5582: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgaatcatttaagagcagaaggacccgcatcagtgttagcgataggtacagctaacccagagaatatcttaatccaagatgaatttcctgactactatttccgtgttactaaatcggaacatatgactcaacttaaagagaagttccggaaaatctgcgataaatccatgatccgaaagagaaactgtttccttaacgaagaacatctcaagcaaaacccgaggttggtagagcacgaaatgcagaccttggatgctaggcaggacatgttggtggtcgaagtgccaaaactcggcaaggacgcgtgcgctaaggcaatcaaggagtggggtcaaccgaagtctaaaatcacgcatctaatatttacatctgcactgacaaccgacatgccgggtgccgattatcactgcgccaagctacttggattgagtccactggttaagagagttatgatgtatcaattggggtgttacggagggggcacagtcctcagaattgctaaggatattgcggaaaataacaagggcgcgagggtccttgctgtatgttgtgatattatggcctgtttgtttcgcgggccctcggattcagatttggaattgcttgtcggacaggcaatttttggtgacggggccgcagcagtcatagtgggagccgaaccagacgaaagcgtgggtgaaagaccaatctttgagttggttctgaccggacaaacgatcttacctaactcggaaggtacgattggaggacatattagagaagccggcctaattttcgatcttcacaaagacgttccaatgttaatctccaataacatagaaaagtgcttgatagaagcatttactcccattggtattagtgactggaacagcattttctggatcacccaccctggaggaaaagctatactcgataaggttgaagagaaactcgacttgaaaaaggagaaattcgttgactcacgacatgtgttatcagagcacgggaatatgagttcatccacagtcttgttcgtaatggatgaattgcgaaaacgctctcttgaggagggaaagagcacaaccggtgacgggtttgagtggggcgtgctattcggttttggcccaggtttgactgtcgagcgggttgttgttcgtagtgtaccaattaagtacggaagaagggcaaagttgtgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 165 - DNA, 5606 bp, synthetic construct
  misc_feature 1..5606: note = pLD56
  source 1..5606: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatggggttgtccttagtttgtacgttcagtttccaaactaactaccacacactactaaatccgcacaacaaaaacccgaaaaattcattgctctcctatcagcacccaaaaacacccattatcaagtctagttacgacaactttccatcaaaatactgtctaacgaaaaactttcatttgttgggcttaaattctcataatcgtatttccagtcagtcccgatcgatcagggccgggagtgaccaaattgaaggttctccacatcatgaaagtgacaattcaattgctacgaagattttaaactttgggcatacatgctggaagctacagcgaccgtatgtagttaaggggatgatcagcattgcctgcggcctattcggaagggaactcttcaataatagacatcttttttcttggggtttaatgtggaaagcttttttcgctttggttcctatccttagttttaacttcttcgccgctattatgaatcaaatttacgatgttgacatcgaccgtattaacaaacccgatctcccccttgtttcaggcgagatgtccattgaaacggcatggattttgtccatcattgttgcgcttactggcttgattgttaccattaagcttaaaagcgctcccttgttcgtttttatatacattttcggcatttttgccggattcgcatacagtgtcccgcctatacgttggaaacaatatccattcacgaacttcttgatcacgatctcatcacatgttggattggcctttacgtcctacagtgctaccacatctgcccttggattgcctttcgtttggaggcctgccttctcgtttatcattgcatttatgacagtgatgggaatgactatcgcatttgctaaagatatcagcgacatagagggcgatgcaaaatatggggtgagtactgttgcgacgaagttgggcgcccgaaatatgaccttcgttgtttccggcgttcttttacttaactatttagtatcgattagcatcgggatcatctggccacaggtgtttaaatcaaatattatgatcttgtcgcatgccatcctagctttctgtcttatatttcaaacaagagaattagccctagcgaactacgcctcagcaccaagtcgtcagttcttcgaatttatatggctactctactacgccgaatacttcgtctatgtcttcatttagtaataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 166 - DNA, 5071 bp, synthetic construct
  misc_feature 1..5071: note = pLD87
  source 1..5071: mol_type = other DNA
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctttaattaagtcgacttgatgtttagagtgtccagatccgcaagatcggctcgcacttgtgttgtgttgtttcaaatcagcctgtcgttttgtgtcgtttgagatcattctgtctcactcttaggctcgcttagaaccgacaacggagaatccgggctcggtttttcggtcggccttgatctgggccttggacttgtactggtcggccatctccacgttgaccagctccttgaccttgtagagctgaccggcgataccaggagacaccttgtagtacttctgggagccgaccttgcccagaccgagggtcttgagcacgtcacgtgttctccacggcattcgcaggatagatcggacctgtgtgactttgtagaacatggcgtttcaggtggttgcgtgagtgtgtaaaatcgtgtctttcagaagttacaaatttcaccgcatttagagtttatgcagatgggcggtgtgtggttgggagttcgatttccgtgcgtgcatttgatcttgatgaattggatttgtacatggggaatagcacgtcaagaaccgcctactgcaaactcgtgaatattgagattattgaggaaattcaaggaaaattcagatcagatttgagagcaaagtccaacaatactacacaatccctttcctgtattcttccaccatcgtcatcgtcgtctgtcttctcttcagctttttaatttcactccccacaaacccaaatttagctgcatcattcatcaacctccaattataactatacatcgcgacacgaacacgaaacacgaaccacgaaccgccgctttgtcgacggagtgagacgggagagagaccatggtctctcgctcgtctcacgcttagcggccgcgtcgacataacttcgtatagcatacattatacgaagttattttctaatttggaccgatagccgtatagtccagtctatctataagttcaactaactcgtaactattaccataacatatacttcactgccccagataaggttccgataaaaagttgtgcagactaaatttatttcagtctcctcttcaccaccaaaatgccctcctacgaagcgcgagctaacgtccacaagtccgcctttgccgcccgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggaactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggatgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagatgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtcatttaactgtgtatatagagagcgtgcaagtatggagcgcttgttcagcttgtatgatggtcagacgacctgtctgatcgagtatgtatgatactgcacaacctgataacttcgtatagcatacattatacgaagttatctcgagggatccctagggaggcacatctaaacgaataacgaatattaatgataccatcatatctcagaacatgtatgactgctgcttccaaacgatatgaggatgagtcctctttcagattaagatagagtacaaatatattatctatatactggtgtctgtgcgatgtcgtatgagcggtgaatcatgtgactgtcacgtggtttggcccaagttacaccgtagctacgcctttcttgaccgactccatggtcttctgggcgggttgacagtttccactggatgagcgtccgcctcctgttcctgtcgttgtccctgcagctcagcctcaatcttctgaccgagctcggagtccagggaaatgccaacaggttgtccaagcaacatcatggtttggtgggcagccgtgatctcatcgtcgttggataccattcggtacttggcctcaatctgcacaaagtagcggtaccactggtttcgagcaaaccgctccaattgagcctctccgtcgagagagagagtaggtgattgctccaacttgcggccaaaatgaagttctcgactcacctttttgaagcggttcttcttgcccatcttggtggcgaaagtagtggctagtggtggatgactttgtataatgtaccgatgaagagggttgtatttgctcagtaagaagtagcgagtgaaatcagatcacttaacgagagcaaagggcaatggaatacctgctgcctgattaacaacagcttctgtgtcgtttctctcttgtgaatgagtgtgttgctagaggtaggttggcactccaatgttacttaattaa

SEQ ID NO: 167 - DNA, 5096 bp, synthetic construct
  misc_feature 1..5096: note = pLD101
  source 1..5096: mol_type = other DNA
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctttaattaacgatccgcgtaaattcagagcaatgccaagatccttatttccgataacagcgagatacacatcagaagaggtggagactctggccccgcacgaccaccacgtcgtcactcaaagcaaagtcacgatgtagaaatcacaatcgtccccataagcacgtggattcccctggttttgtttcggcctcggcagtggcaattctggggtatataaaccagcaaggttatgacctgattgacctgcttgtggcctgataaaccgcctgacttttgtgataaggttgggagcatgcggctcgggccattgtgagcattttcgtcaaggactggccaagtccaactggaagagacatgggcaaaatgtcgactttacagatgtacccgaaattgcttcaattgctctcagctgccggaaccggatctatcggtgcaagtatcgtacagtagacatgtgctattggtaaccctcggtattggctaggtttcgtatcagggatacagttcaacgctgatcgcatatggcatgattccggctcgacacagcgaccaagaaccaagcgtgtatgtcgtagacttgcaaatcatgtggggcttatccccggatttccccaagtcacgttttcacaaaggctgtctcccgaatgcatgagccgaggcaggctaaactggtttgttcatgtaccccacacaacgtaaagatgcaccccatgtgcagtgaaataccacaagtatatatataccgacctacccgagatagcaaattgattctacacttacactaccaattcttacatcaaaccaaaccgcttgagaccatccggtctctgatcatcctcgagataacttcgtataatgtatgctatacgaagttatcaggttgtgcagtatcatacatactcgatcagacaggtcgtctgaccatcatacaagctgaacaagcgctccatacttgcacgctctctatatacacagttaaatgacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcatctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacatcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagttccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgggcggcaaaggcggacttgtggacgttagctcgcgcttcgtaggagggcattttggtggtgaagaggagactgaaataaatttagtctgcacaactttttatcggaaccttatctggggcagtgaagtatatgttatggtaatagttacgagttagttgaacttatagatagactggactatacggctatcggtccaaattagaaaataacttcgtataatgtatgctatacgaagttatgtcgacgcggccgcacaagcactacatggacgaggtcaaggctgccaacaaccctcgtaacacccatgctccttactacgagacaaagctgcgacccttcctgttccgacccgatgaggacgaggagatttgcgacctggacgagtaggttgttgtaatactatgatttattgtgtttatatgttattgatactattgaaagagttattgtgtaattttagatgctgtatgttaactagaagctcagattctacaaagagatcctcagatctgaggaatgatctacgttctgcaatagaagggacaactgcagctctgaatgaccacaaaaagaatacaccacaagcagttgtaactgagctattagccttgctttcgcaccattcgcttttctggatggtagccctttactacaagtagctaatatggaatgtacattaccgtctcattacaatatgtatatgcaagttcatggcacttcatgcacaccagccccttcgttaagtaccttccaaaaagtgatcaatgataagtgatatatctaatttagagatctggacacacgaacaagtcgggaacacaaatcccgagatgattgcctgctcagaggagtccaattagtctttacaccattcgtgctaatgaagggcacagaatattccactttgaaagtttaagattaagctcggctcgcaattatgcatgaaaaatatgtagggagagaacgatcccacgagttctgtttggttgcgagagtgttcgggttttctcctaaaaagaataggggagggaaaaattatcgcctaagtcaccattaattaa

SEQ ID NO: 168 - DNA, 5096 bp, synthetic construct
  misc_feature 1..5096: note = pLD102
  source 1..5096: mol_type = other DNA
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctttaattaattggcaaattttactgtggccttcagaacggtaaaaatagaccaatcagaattctgaaaagcacatcttgatctcctcattgcggggagtccaacggtggtcttattcccccgaatttcccgctcaatctcgttccagaccgacccggacacagtgcttaacgccgttccgaaactctaccgcagatatgctccaacggactgggctgcatagatgtgatcctcggcttggagaaatggataaaagccggccaaaaaaaaagcggaaaaaagcggaaaaaaagagaaaaaaaatcgcaaaatttgaaaaatagggggaaaagacgcaaaaacgcaaggaggggggagtatatgacactgataagcaagctcacaacggttcctcttatttttttcctcatcttctgcctaggttcccaaaatcccagatgcttctctccagtgccaaaagtaagtaccccacaggttttcggccgaaaattccacgtgcagcaacgtcgtgtggggtgttaaaatgtgggggcggggaaccaggacaagaggctcttgtgggagccgaatgagagcacaaagcgggcgggtgtgataagggcatttttgcccattttcccttctcctgtctctccgacggtgatggcgttgtgcgtcctctatctatttctttttatttctttttgttttatttctctgactaccgatttggcttgatttcctcaaccccacacaaataagctcgggccgaggaatatatatatacacggacacagtcgccctgtggacaacacgtcactacctctacgacgcttgagaccatccggtctctgatcatcctcgagataacttcgtataatgtatgctatacgaagttatcaggttgtgcagtatcatacatactcgatcagacaggtcgtctgaccatcatacaagctgaacaagcgctccatacttgcacgctctctatatacacagttaaatgacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcatctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacatcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagttccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgggcggcaaaggcggacttgtggacgttagctcgcgcttcgtaggagggcattttggtggtgaagaggagactgaaataaatttagtctgcacaactttttatcggaaccttatctggggcagtgaagtatatgttatggtaatagttacgagttagttgaacttatagatagactggactatacggctatcggtccaaattagaaaataacttcgtataatgtatgctatacgaagttatgtcgacgcggccgcatggagcgtgtgttctgagtcgatgttttctatggagttgtgagtgttagtagacatgatgggtttatatatgatgaatgaatagatgtgattttgatttgcacgatggaattgagaactttgtaaacgtacatgggaatgtatgaatgtgggggttttgtgactggataactgacggtcagtggacgccgttgttcaaatatccaagagatgcgagaaactttgggtcaagtgaacatgtcctctctgttcaagtaaaccatcaactatgggtagtatatttagtaaggacaagagttgagattctttggagtcctagaaacgtattttcgcgttccaagatcaaattagtagagtaatacgggcacgggaatccattcatagtctcaattttcccataggtgtgctacaaggtgttgagatgtggtacagtaccaccatgattcgaggtaaagagcccagaagtcattgatgaggtcaagaaatacacagatctacagctcaatacaatgaatatcttctttcatattcttcaggtgacaccaagggtgtctattttccccagaaatgcgtgaaaaggcgcgtgtgtagcgtggagtatgggttcggttggcgtatccttcatatatcgacgaaatagtagggcaagagatgacaaaaagtatctatatgtagacagcgtagaatatggatttgattggtataaattcatttattgcgtgtctcacaaatactctcgataagttggggttaaactggagatggaacaatgtcgatatctcgacatattttgatatttgttaattaa

SEQ ID NO: 169 - DNA, 6044 bp, synthetic construct
  misc_feature 1..6044: note = pLD111
  source 1..6044: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgaattgttcagcatttagtttttggtttgtttgtaagattattttcttctttttgtcatttaacattcaaatttcaattgcaaacccacaagaaaactttttgaagtgtttttcagaatacattccaaacaatccagctaacccaaagtttatttacacacaacatgatcaattgtacatgtcagttttgaactcaacaattcaaaacttgagatttacatcagataccacaccaaagccattggttattgttacaccatcaaacgtttcccatattcaagcatcaatcttgtgttcaaagaaggttggattgcaaattagaaccagatcaggaggacacgatgcagaaggaatgtcatacatttcacaagttccattcgttgttgttgatttgagaaacatgcactcaattaagattgatgttcattcacaaacagcatgggttgaagcaggagcaacattgggtgaagtttactactggattaacgaaaagaacgaaaacttcagttttccaggaggttactgtccaacagttggagttggaggacatttttcaggtggaggatacggagcattgatgagaaactacggattggcagcagataacattattgatgcacacttggttaacgttgatggaaaggttttggatagaaagtcaatgggagaagatttgttttgggcaattagaggaggtggtggagagaactttggaattattgcagcatggaagatcaagttggttgcagttccatcaaagtcaacaatcttttcagttaagaagaacatggaaattcatggtttggttaagttgtttaacaagtggcaaaacattgcatacaagtacgataaggatttggttttgatgacacattttattacaaagaacattacagataaccatggaaagaacaagacaacagttcacggatacttttcatcaatttttcacggaggagttgattcattggttgacttgatgaacaagtcatttccagaattgggaatcaagaagacagattgtaaggaattttcatggattgatacaacaattttctactcaggagttgttaactttaacacagcaaactttaagaaggaaattttgttggacagatcagcaggaaagaagaccgcattttccattaagttggattacgttaagaaaccaattccagaaacagcaatggttaagattttggaaaagttgtacgaagaagatgttggtgttggaatgtacgttttgtacccatacggaggaattatggaagaaatctcagaatcagcaattccatttccacatagagcaggtattatgtacgaattgtggtacacagcatcatgggaaaagcaagaagataatgaaaagcatattaactgggttagatcagtttacaactttacaacaccatacgtttcacaaaacccaagattggcatacttgaactacagagatttggatttgggaaagacaaacccagaatcaccaaacaactatacacaagctagaatttggggagaaaagtactttggtaagaacttcaacagattggttaaagttaagacaaaggcagatccaaataacttctttagaaacgaacaatcaattccaccattgccaccacatcatcattaataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 170 - DNA, 6044 bp, synthetic construct
  misc_feature 1..6044: note = pLD112
  source 1..6044: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgaattgtagcactttctcattctggtttgtttgtaagattattttctttttcttgtcatttaacattcaaatttcaattgcaaacccacaagagaactttttgaagtgtttctcagaatacattccaaacaacccagctaacccaaagtttatttacacccaacacgatcaattgtacatgtcagttttgaactcaacaattcaaaacttgagatttacatcagatacaacaccaaagccattggttattgttacaccatcaaacgttagtcatattcaagcatcaatcttgtgttcaaagaaggttggattgcaaattagaactagatcaggaggacatgatgcagaaggattgtcatacatttcacaagttccatttgcaattgttgatttgagaaacatgcacacagttaaggttgatattcattcacaaacagcatgggttgaagcaggagcaacattgggtgaagtttactactggattaacgaaatgaacgaaaacttctcatttccaggaggatactgtccaacagttggtgttggaggacacttttcaggtggtggatacggagcattgatgagaaactacggattggcagcagataacattattgatgcacatttggttaacgttgatggaaaggttttggatagaaagtcaatgggagaagatttgttttgggcaattagaggaggtggaggagaaaactttggaatcattgcagcatgtaagatcaagttggttgttgttccatcaaaggcaacaatcttttcagttaagaagaacatggaaatccatggattggttaagttgtttaacaagtggcaaaacattgcatacaagtacgataaggatttgatgttgacaacacattttagaacaagaaacattacagataaccacggaaagaataagacaacagttcatggatacttttcatcaattttcttgggaggagttgattcattggttgacttgatgaacaagagttttccagaattgggaatcaagaagacagattgtaaggaattgtcatggatcgatacaaccattttctactcaggagttgttaactacaacacagctaactttaagaaggaaattttgttggacagatcagcaggtaaaaagacagcattttcaattaagttggattacgttaagaaattgattccagaaacagcaatggttaagattttggaaaagttgtacgaagaagaagttggagttggaatgtacgttttgtacccatacggaggaattatggatgaaatttcagaatcagcaattccatttccacatagagcaggtattatgtacgaattgtggtacacagcaacatgggaaaagcaagaagataacgaaaagcatattaactgggttagatcagtttacaactttacaaccccatacgtttcacaaaacccaagattggcatacttgaactacagagatttggatttgggaaagacaaacccagaatcaccaaataactacacacaagctagaatttggggagaaaagtactttggtaagaactttaacagattggtgaaggttaagacaaaggcagacccaaacaatttctttagaaacgaacaatcaattccaccattgccaccaagacatcattaataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 171 - DNA, 7167 bp, synthetic construct
  misc_feature 1..7167: note = pLD113
  source 1..7167: mol_type = other DNA
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctgatcccaatattacacccaagtagcatgcataagctaaaagtaactcgcagcgcacaccgtgcagattcataagtctatgattaattgaacgccaataacccggcttactacaagtacaagtaggtatacatagcggtaatgaatcattagaaaaataaaaaacaaaaaaaaacaaaacaaactgttgtggatgcatcaacagtagtacatagttgtacgatgtacttgtacttgtaaaagcaaaaatgtacaatatctcagggagcgcaacttttacgttcgaagaacaatgtaccgcataccgcattctagattctgcggaacgtctaacctggaaatacgattttttatttctttcattttttttgcttcttcaaaagtatggtaatttcctaccattacagttgacactgaacgaggggggattgaatttaagcaaaaaattaaatcaaaatacctttatgtatccagcccatgtaataaacaaaaggattatataacaagaaataaatatatacctttaatggatcattagaataaaaataaatacgagaagcacaccagagaagctttttgattgccactataccgctactttggtatatcttattataattgttgaatttgcaagatagaatgtcattcattggagagaaatccaaggaatatgtgggatgaaatgactagaagtatgaacaatgagaatagtacatacttgtacctgtatttctagaagagagaaagacagttgagtgtgtgattctcgtccaataataatctcaatagtaacgtgtgaatagctgttctttgatagttgatatttctcgatgactatttatgttgtacaagggatttttttcgttgctgttgatttcgaattaggcaatgcagatatcatttatgctatccatatttaagatttcccatacgcatttataacatttattctacataaattgttaaatgaacgaactgccattataaattgtttcctaaataggaagtgtttttcataaagcaagtaagttgtctaataatactaagtaataaaaataagttcatacaatatattttgagaacatcatttggaggcggtagatggagtctgtttattattaaacaatgcgagatgaccccttaaatattgagaacatcagttggaggcggcagatggagtctgtctatttagcaatgggacatgactgtcagtatcatcatatgtatatatataatacatataatattatataacacgatttttttaaattattggcccgaaaattaatcagtgtagactggatcctcgagaaccatttaattaacaagtcgagaacgtaccactgtcctccactacaaacacacccaatctgcttcttctagtcaaggttgctacaccggtaaattataaatcatcatttcattagcagggctgggccctttttatagagtcttatacactagcggaccctgccggtagaccaacccgcaggcgcgtcagtttgctccttccatcaatgcgtcgtagaaacgacttactccttcttgagcagctccttgaccttgttggcaacaaagtctccgacctcggaggtggaggaggagcctccgatatcggcggtagtgataccagcctcgacggactccttgacggcagcctcaacagcgtcaccggcgggcttcatgttaagagagaacttgagcatcatggcggcagacagaatggtggcaatggggttgaccttctgcttgccgagatcgggggcagatccgtgacagggctcgtacagaccgaacgcctcgttggtgtcgggcagagaagccagagaggcggagggcagcagacccagagaaccggggatgacggaggcctcgtcggagatgatatcgccaaacatgttggtggtgatgatgataccattcatcttggagggctgcttgatgaggatcatggcggccgagtcgatcagctggtggttgagctccagctgggggaattcgtccttgaggactcgggtgacggtctttcgccaaagtcgagaggaggccagcacgttggccttgtcaagggaccacacgggaagaggggggttgtgctgaagggccaggaaggcggccattcgggcaattcgctcaacctcaggaacggagtaagtctcagtgtcggaagcgacgccagatccgtcatcctcctttcgctctccaaagtagatacctccgacgagctctcggacaatgatgaagtcggtgccctcaacgtttcggatgggggagagatcggcgagcttgggcgacagcagctggcagggtcgcaggttggcgtacaggttcaggtcctttcgcagcttgagaagaccctgctcgggtcgcacgtcggttcgtccgtcgggagtggtccatacggtgttggcagcgcctccgacagcaccgagcataatagagtcagcctttcggcagatgtcgagagtagcgtcggtgatgggctcgccctccttctcaatggcagctcctccaatgagtcggtcctcaaacacaaactcggtgccggaggcctcagcaacagacttgagcaccttgacggcctcggcaatcacctcggggccacagaagtcgccgccgagaagaacaatcttcttggagtcagtcttggtcttcttagtttcgggttccattgtggatgtgtgtggttgtatgtgtgatgtggtgtgtggagtgaaaatctgtggctggcaaacgctcttgtatatatacgcacttttgcccgtgctatgtggaagactaaacctccgaagattgtgactcaggtagtgcggtatcggctagggacccaaaccttgtcgatgccgatagcgctatcgaacgtaccccagccggccgggagtatgtcggaggggacatacgagatcgtcaagggtttgtggccaactggtaaataaatgatgactcaggcgacgacggaattcgacagcaactactcctttcaccaaccatgtgcattttagctcgaataacattcacaggcttggtgatctacatccatggtgtctggccgattaccgtggtgttttggcagtaacgagaatattgagtgaactcttcccatcaccaataaagactcatactacaatcacgagcgcttcagctgccactatagtgttggtgacacaatacccctcgatgctgggcattactgtagcaagagatattatttcatggcgcattttccagtctacctgactttttagtgtgatttcttctccacattttatgctcagtgtgaaaagttggagtgcacacttaattatcgccggttttcggaaagtactatgtgctcaaggttgcaccccacgttacgtatgcagcacattgagcagcctttggaccgtggagataacggtgtggagatagcaacgggtagtcttcgtattaattcaatgcattgttagttttatatgatatggtgtcgagcggccgcgaccgggttggcggcgcatttgtgtcccaaaaaacagccccaattgccccaattgaccccaaattgacccagtagcgggcccaaccccggcgagagcccccttctccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaatgtccaacctcctcaccgtccaccagaaccttcccgcccttcccgtggacgccacctccgacgaggtccgaaagaacctcatggacatgttccgagatcgacaggccttctccgagcacacttggaagatgctcctctccgtctgccgatcctgggccgcctggtgcaagctcaacaaccgaaagtggttccccgccgagcccgaggacgtccgagactacctcctttacctccaggcccgaggcctcgccgtcaagaccatccagcagcacctcggccagctcaacatgctccaccgacgatccggcctcccccgaccctccgactccaacgccgtgtccctcgtcatgcgacgaatccgaaaggagaacgtggacgccggcgagcgagccaagcaggcccttgccttcgagcgaaccgacttcgaccaggtccgatccctcatggagaactccgaccgatgccaggacatccgaaacctcgccttccttggcatcgcctacaacaccctccttcgaatcgccgagatcgcccgaatccgagtcaaggacatctcccgaaccgacggcggccgaatgctcatccacatcggccgaaccaagaccctcgtgtccaccgccggcgtcgagaaggccctctccctcggcgtcaccaagctcgtcgagcgatggatctccgtgtccggcgtcgctgacgaccccaacaactacctcttctgccgagtccgaaagaacggcgtcgctgctccctccgccacctcccagctctccacccgagcccttgagggcatcttcgaggccacccaccgactcatctacggcgccaaggacgactccggccagcgatacctcgcctggtccggccactctgctcgagtcggtgccgcccgagacatggcccgagccggtgtctccatccccgagatcatgcaggccggcggctggaccaacgtcaacatcgtcatgaactacatccgaaacctcgactccgagactggcgccatggtccgacttcttgaggacggcgactgatgatcatatgattacattaatagctaattacgtgtatccgatatatatactaattacaatagtacatattagaacatacaatagttttaattaa

SEQ ID NO: 172 - DNA, 6125 bp, synthetic construct
  misc_feature 1..6125: note = pLD125
  source 1..6125: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgttcttgaaacacatttttgttgctctcgcttttgccttgttagctgacgctaccccagcccagaagagatctcccggcttcgttgctttagactttgacatcgtcaaggttcaaaagaacgtgactgccaacgacgacgccgctgccattgttgccaagagacagaccaacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacactgagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 173 - DNA, 6230 bp, synthetic construct
  misc_feature 1..6230: note = pLD127
  source 1..6230: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgcaattgtccttgtcggttttatcaaccgttgccacggccttgttgtccctaaccaccgccgtcgatgctaagtcccacaacatcaagttgtccaagttgtccaacgaagaaacattggacgcctccacattccaagaatacacgagctccttggccaacaagtacatgaacttgttcaacgccgctcacggtaacccaaccagctttggcttgcaacacgtcttgtccaaccaagaagctgaagtcccattcgttaccccacaaaagggtggcaacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacactgagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 174 - DNA, 8110 bp, synthetic construct
  misc_feature 1..8110: note = pLD131
  source 1..8110: mol_type = other DNA
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctgatcccaatattacacccaagtagcatgcataagctaaaagtaactcgcagcgcacaccgtgcagattcataagtctatgattaattgaacgccaataacccggcttactacaagtacaagtaggtatacatagcggtaatgaatcattagaaaaataaaaaacaaaaaaaaacaaaacaaactgttgtggatgcatcaacagtagtacatagttgtacgatgtacttgtacttgtaaaagcaaaaatgtacaatatctcagggagcgcaacttttacgttcgaagaacaatgtaccgcataccgcattctagattctgcggaacgtctaacctggaaatacgattttttatttctttcattttttttgcttcttcaaaagtatggtaatttcctaccattacagttgacactgaacgaggggggattgaatttaagcaaaaaattaaatcaaaatacctttatgtatccagcccatgtaataaacaaaaggattatataacaagaaataaatatatacctttaatggatcattagaataaaaataaatacgagaagcacaccagagaagctttttgattgccactataccgctactttggtatatcttattataattgttgaatttgcaagatagaatgtcattcattggagagaaatccaaggaatatgtgggatgaaatgactagaagtatgaacaatgagaatagtacatacttgtacctgtatttctagaagagagaaagacagttgagtgtgtgattctcgtccaataataatctcaatagtaacgtgtgaatagctgttctttgatagttgatatttctcgatgactatttatgttgtacaagggatttttttcgttgctgttgatttcgaattaggcaatgcagatatcatttatgctatccatatttaagatttcccatacgcatttataacatttattctacataaattgttaaatgaacgaactgccattataaattgtttcctaaataggaagtgtttttcataaagcaagtaagttgtctaataatactaagtaataaaaataagttcatacaatatattttgagaacatcatttggaggcggtagatggagtctgtttattattaaacaatgcgagatgaccccttaaatattgagaacatcagttggaggcggcagatggagtctgtctatttagcaatgggacatgactgtcagtatcatcatatgtatatatataatacatataatattatataacacgatttttttaaattattggcccgaaaattaatcagtgtagactggatcctcgagaaccatttaattaagatcgtcgacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgcttgagtatcgaagtcttgtacctccctgtcaatcatggcaccactggtcttgacttgtctattcatactggacaagcgccagagttaagcttgtagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggaggcctaattttgacgtgcaaggggcgaattggggcgagaaacacgtcgtggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacgacgccatgggcctctctctagtatgtaccttctctttccagaccaactatcacactctactgaacccccataacaagaaccctaaaaattctcttctcagttaccagcaccccaagacgcctatcattaagtcctcctacgacaactttccctctaagtactgcctgaccaaaaacttccatctcctgggactgaactctcataacagaattagtagccagtcccgatctatccgagctggctctgaccagattgagggctcccctcaccatgaatccgacaacagcatcgctaccaagattttgaattttggtcacacatgctggaagctccagcgaccgtacgtcgtgaagggtatgatctcgattgcctgtggactgttcggacgtgagctttttaataatcgacacttgttttcatggggcctcatgtggaaggcttttttcgccctcgtgcccattctgtctttcaacttctttgccgctattatgaaccaaatctacgacgttgatattgataggatcaacaagcctgacctgccgctcgtctcgggggagatgtctatcgagacagcgtggattctttcgattatcgtcgcgctgactggccttatcgttaccataaagttgaagtctgcacccctcttcgtgtttatctacattttcggtatttttgctggattcgcgtactccgttccccctatcagatggaagcagtacccctttactaactttctgattactatcagcagccacgtcggtttagcctttacctcatattcggccaccaccagtgcactgggcctccccttcgtctggcgacctgcattttcattcatcatcgccttcatgactgtgatgggtatgaccatcgctttcgctaaggacatctccgacatcgagggtgatgctaaatatggagtgtccaccgtggccactaagctgggagcccggaacatgacgttcgtcgtctctggtgttctgctccttaactacttggtttcgatctccattggcattatctggccacaagtcttcaagtccaacattatgattctgtcccacgccattcttgccttttgcctgatcttccagacacgcgaactcgctctcgctaactacgcctccgccccatcgcgacagttcttcgagttcatctggctgctttactacgccgagtacttcgtttacgtgttcatctaataagagtaggcaattaacagatagtttgccggtgataattctcttaacctcccacactcctttgacataacgatttatgtaacgaaactgaaatttgaccagatattgttgtaaatagaaaatctggcttgtaggtggcaaaatgcggcgtctttgttcatcaattccctctgtgactactcgtcatccctttatgttcgactgtcgtatttcttattttccatacatatgcaagtgagatgcccgtgtccgaattcgctatggatccatagcgtcgacaccatatcatataaaactaacaatgcattgaattaatacgaagactacccgttgctatctccacaccgttatctccacggtccaaaggctgctcaatgtgctgcatacgtaacgtggggtgcaaccttgagcacatagtactttccgaaaaccggcgataattaagtgtgcactccaacttttcacactgagcataaaatgtggagaagaaatcacactaaaaagtcaggtagactggaaaatgcgccatgaaataatatctcttgctacagtaatgcccagcatcgaggggtattgtgtcaccaacactatagtggcagctgaagcgctcgtgattgtagtatgagtctttattggtgatgggaagagttcactcaatattctcgttactgccaaaacaccacggtaatcggccagacaccatggatgtagatcaccaagcctgtgaatgttattcgagctaaaatgcacatggttggtgaaaggagtagttgctgtcgaattccgtcgtcgcctgagtcatcatttatttaccagttggccacaaacccttgacgatctcgtatgtcccctccgacatactcccggccggctggggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgtttgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtcttctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacactgagacttactccgttcctgaggttgagcgaattgcccgaatggccgccttcctggcccttcagcacaacccccctcttcccgtgtggtcccttgacaaggccaacgtgctggcctcctctcgactttggcgaaagaccgtcacccgagtcctcaaggacgaattcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcctcctccacctccgaggtcggagactttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggcccagccctgctaatgaaatgatgatttataatttaccggtgtagcaaccttgactagaagaagcagattgggtgtgtttgtagtggaggacagtggtacgttttaattaa

SEQ ID NO: 175 - DNA, 7557 bp, synthetic construct
  misc_feature 1..7557: note = pLD132
  source 1..7557: mol_type = other DNA
ttaattaagatcgtcgacgattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgcttgagtatcgaagtcttgtacctccctgtcaatcatggcaccactggtcttgacttgtctattcatactggacaagcgccagagttaagcttgtagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggaggcctaattttgacgtgcaaggggcgaattggggcgagaaacacgtcgtggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacgacgccatgaagtgttcgacgttttctttttggtttgtttgtaaaatcattttctttttcttttctttcaacatccaaacgtcgatcgcaaaccctagagagaactttcttaagtgcttctcgcagtacatccctaataacgctaccaaccttaagctggtgtacacccagaacaaccctctttacatgtctgttctaaacagcaccatccacaatcttagattcacatcagacaccactcccaagccgctcgtcatcgtgaccccgagtcatgtgtcccatatccaaggcactatcctgtgctctaaaaaggtcggtctgcagattcggactcgctccggtggacatgattcggagggcatgtcctacattagccaggtcccctttgtgatcgtggacctgaggaacatgcggtctattaagattgatgtgcactcacagaccgcttgggtcgaggctggtgcgacattgggtgaggtgtactactgggtgaacgagaagaacgagaacctgagcctcgccgctggctactgtcccaccgtttgtgccggtggacacttcggcggaggcggatacggtccacttatgcgaaactacgggctcgcagctgataatatcatcgacgcacaccttgttaacgttcacggcaaggtgctggaccgaaaaagcatgggtgaggacctattttgggccttgcgaggcggtggtgccgaatccttcggaattatcgtggcctggaagatccgactggtcgctgtgccaaagtccactatgttctccgtcaagaaaattatggagatccacgaactcgtaaagctcgtcaataagtggcagaacatcgcctacaagtatgacaaggatctgctgctcatgactcacttcatcacgcgaaacattacagacaaccagggaaagaacaagaccgctatccatacctacttctcctctgtcttccttgggggtgtcgattccctcgttgatctcatgaacaaatcttttccagagctcggaatcaagaagaccgactgccgacagctctcttggatcgacaccattattttctactcaggagtcgtaaactacgatactgacaactttaacaaggagattctgttagatcgatcggccggccagaacggtgccttcaagatcaagctcgactatgtcaaaaagcccattcctgaatccgtcttcgttcaaattcttgaaaagttgtacgaggaggatatcggcgccggaatgtacgcgctgtacccctacggtggcattatggacgagatttctgaaagtgctattcccttcccccaccgtgctggcattctgtatgagctgtggtacatttgctcctgggaaaagcaggaggacaacgagaagcacttgaactggatacgaaacatttacaatttcatgaccccctatgtttcgaagaaccctcgactggcctacctgaattaccgcgacctcgacatcggaattaacgaccctaagaaccccaataactatactcaggccagaatctggggcgagaagtacttcggcaagaactttgaccgtctggttaaggtcaagaccctcgtggaccctaacaacttcttccgaaacgagcagtctatcccccctctgccccgacaccggcattaataagagtaggcaattaacagatagtttgccggtgataattctcttaacctcccacactcctttgacataacgatttatgtaacgaaactgaaatttgaccagatattgttgtaaatagaaaatctggcttgtaggtggcaaaatgcggcgtctttgttcatcaattccctctgtgactactcgtcatccctttatgttcgactgtcgtatttcttattttccatacatatgcaagtgagatgcccgtgtccgaattcgctatggatccatagcgcaggttgtgcagtatcatacatactcgatcagacaggtcgtctgaccatcatacaagctgaacaagcgctccatacttgcacgctctctatatacacagttaaatgacatatccatagtctaacctctaacagttaatcttctggtaagcctcccagccagccttctggtatcgcttggcctcctcaataggatctcggttctggccgtacagacctcggccgacaattatgatatccgttccggtagacatgacatcctcaacagttcggtactgctgtccgagagcatctcccttgtcgtcaagacccaccccgggggtcagaataagccagtcctcagagtcgcccttaggtcggttctgggcaatgaagccaaccacaaactcggggtcggatcgggcaagctcaatggtctgcttggagtactcgccagtggccagagagcccttgcaagacagctcggccagcatgagcagacctctggccagcttctcgttgggagaggggaccaggaactccttgtactgggagttctcgtagtcagagacatcctccttcttctgttcagagacagtttcctcggcaccagctcgcaggccagcaatgattccggttccgggtacaccgtgggcgttggtgatatcggaccactcggcgattcggtagacaccgttcttgtactggtgcttgacagtgttgccaatatctgcgaactttctgtcctcgaacaggaagaaaccgtgcttaagagcaagttccttgagggggagcacagttccggcgtaggtgaagtcgtcaatgatgtcgatatgggtcttgatcatgcacacataaggtccgaccttatcggcaagctcaatgagctccttggtggtggtaacatccagagaagcacacaggttggttttcttggctgccacgagcttgagcactcgggcggcaaaggcggacttgtggacgttagctcgcgcttcgtaggagggcattttggtggtgaagaggagactgaaataaatttagtctgcacaactttttatcggaaccttatctggggcagtgaagtatatgttatggtaatagttacgagttagttgaacttatagatagactggactatacggctatcggtccaaattagaaattaattaaatggttctcgaggatccagtctacactgattaattttcgggccaataatttaaaaaaatcgtgttatataatattatatgtattatatatatacatatgatgatactgacagtcatgtcccattgctaaatagacagactccatctgccgcctccaactgatgttctcaatatttaaggggtcatctcgcattgtttaataataaacagactccatctaccgcctccaaatgatgttctcaaaatatattgtatgaacttatttttattacttagtattattagacaacttacttgctttatgaaaaacacttcctatttaggaaacaatttataatggcagttcgttcatttaacaatttatgtagaataaatgttataaatgcgtatgggaaatcttaaatatggatagcataaatgatatctgcattgcctaattcgaaatcaacagcaacgaaaaaaatcccttgtacaacataaatagtcatcgagaaatatcaactatcaaagaacagctattcacacgttactattgagattattattggacgagaatcacacactcaactgtctttctctcttctagaaatacaggtacaagtatgtactattctcattgttcatacttctagtcatttcatcccacatattccttggatttctctccaatgaatgacattctatcttgcaaattcaacaattataataagatataccaaagtagcggtatagtggcaatcaaaaagcttctctggtgtgcttctcgtatttatttttattctaatgatccattaaaggtatatatttatttcttgttatataatccttttgtttattacatgggctggatacataaaggtattttgatttaattttttgcttaaattcaatcccccctcgttcagtgtcaactgtaatggtaggaaattaccatacttttgaagaagcaaaaaaaatgaaagaaataaaaaatcgtatttccaggttagacgttccgcagaatctagaatgcggtatgcggtacattgttcttcgaacgtaaaagttgcgctccctgagatattgtacatttttgcttttacaagtacaagtacatcgtacaactatgtactactgttgatgcatccacaacagtttgttttgtttttttttgttttttatttttctaatgattcattaccgctatgtatacctacttgtacttgtagtaagccgggttattggcgttcaattaatcatagacttatgaatctgcacggtgtgcgctgcgagttacttttagcttatgcatgctacttgggtgtaatattgggatcaggtcaggcggaatggcacttccttattccagatgcgtgcggattatgcatgactggccgtcgttttacaacgtcgtgactgggaaaaccctggcgcttgtggtggatcacagttattggagacttccttggccgtacgatgggttttgtttcaggtatcccaacctggacgtctcgtataatgcgacaaacaacagataaaacgaaaggcccagtctttcgactgagcctttcgttttatttggatccactagttctagagcggcgggggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaatttgttaattggttgtaacactggcagagcggacgatttgaagccccctacccatttacgctgacttgacgggacggcggctttgttgaatagagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttccagcgacatatgccatacgccgaactgaactccacctaatgtcttggagattgtcggatagcgcggaataggattgtgccctctgagcggataacaatttcacacaggaaacagctatgaccatgacacatgcatccaccatcgcagacttatcatcactgagcctccacctagcct

SEQ ID NO: 176 - DNA, 8500 bp, synthetic construct
  misc_feature 1..8500: note = pLD135
  source 1..8500: mol_type = other DNA
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctttaattaattggcaaattttactgtggccttcagaacggtaaaaatagaccaatcagaattctgaaaagcacatcttgatctcctcattgcggggagtccaacggtggtcttattcccccgaatttcccgctcaatctcgttccagaccgacccggacacagtgcttaacgccgttccgaaactctaccgcagatatgctccaacggactgggctgcatagatgtgatcctcggcttggagaaatggataaaagccggccaaaaaaaaagcggaaaaaagcggaaaaaaagagaaaaaaaatcgcaaaatttgaaaaatagggggaaaagacgcaaaaacgcaaggaggggggagtatatgacactgataagcaagctcacaacggttcctcttatttttttcctcatcttctgcctaggttcccaaaatcccagatgcttctctccagtgccaaaagtaagtaccccacaggttttcggccgaaaattccacgtgcagcaacgtcgtgtggggtgttaaaatgtgggggcggggaaccaggacaagaggctcttgtgggagccgaatgagagcacaaagcgggcgggtgtgataagggcatttttgcccattttcccttctcctgtctctccgacggtgatggcgttgtgcgtcctctatctatttctttttatttctttttgttttatttctctgactaccgatttggcttgatttcctcaaccccacacaaataagctcgggccgaggaatatatatatacacggacacagtcgccctgtggacaacacgtcactacctctacgacgctatggatccatagcgaattcggacacgggcatctcacttgcatatgtatggaaaataagaaatacgacagtcgaacataaagggatgacgagtagtcacagagggaattgatgaacaaagacgccgcattttgccacctacaagccagattttctatttacaacaatatctggtcaaatttcagtttcgttacataaatcgttatgtcaaaggagtgtgggaggttaagagaattatcaccggcaaactatctgttaattgcctactcttattacagtttggctcgtcgtccccgagacttgccggccttgatagcgaggagtgtcttctcgtcagcaggggcgctaccggaggctcgttgctgtctgaagtaggttcgggcctcgtcctgtcgggtggcctcagcgccggtggcgatagcggcccgaaggtgctcggctccgtagccgaaggcatcaaccagatcaagtgcgtgtggtcggatcttaacgagaagacgattaatgtaagtgccgaccgttcggcctctctgcatcgagagcctaccgttcatcagataccagctgaggtgcttctcgataagggacagaccaaaaaggtcacgcaatcgggtcaacacttccttagtgccagcgtcgtccaccttggccaaagcctcagtaaaggcctcccactgtaaaagctcagcgtgggcctgggcagcctcgatcagttcgttttgatgctgattgaacagtgctgccgcctggtgctgggggagcttgcccgcgcctttgagagcagcgccaacctctgcaaccatggactggactctgtcggtcagcagagttcgctgaccttcttcgtctcggagggcaagagcagatttctggacagatccactgtcggccacgaactgagcgacctgtcgcagtccggttcggtgtagcgcgactccggcagcctggtccacgacgtaccgcgcgagcacgccgaagttggcacctcggaactccttagcatagtcagcgagcagcctcttggccaccaactgaagaagcacggtgttgtctccttcgaaggtcacgtagacgtcgagatcggcccggagagaagcgaatcggttctcaatcagaaaacctgcacccccacacgcttctcggcactcttgtagggtatcgagagcatgccaggtagaaaggggcttcagagcagcagccagtgtctcaaggtcctgacggtcggcgtcagtatcatgagcacccgagaacacgtcgtcgaatttctgcagcagttgctcatgggcgaacgatgcggcgtaggtggtggcgagtcgggtaaagaggcgccgctggtgtcgctggtaatccaggagcacctcctcctctgtgggcgaagtggcgttgaactgtcttcgctcagcggcgtagtggatggcggattgcagagcaaccttcgatgcagccactgcagcaccgtccaggctgactcggccctggaccagcgtacctagcattgtaaagaatcttcgccccggagattcgatggttgagctgtaggtgccatcgacggcaacatcgccgtaacggttaagcaggttggttcggggaatgcgaacattggtgaaatggagacgtccgttatcaattccgtttagtccccccttaataccgtcgtcctctccaccaataccagggagaaagtctccggtggcgggatctctcagatcgacgtagaaggcgtgcactccatggttaacctttcgagtaatcagttgggcaaacacgacagcggccaaaccgtcgttagcggcgtttccaatgtagtccttccaggcggctcggaacggggtgtcaataacgaactcctgggtttcctcatcataggtggccgttgtagcaatggaagcgacatcggagccatggccagtctcagtcatggcaaagcaaccagggatttccagagacatgatgcctgggagccacttgtcgtggtgctccctagttcccaagtgcataacagcggagccgaacagtccccactggacgccggccttgatctggagggaggggtccgccgtgacaagctcctcaaaaccagcgatgtttccgccgtggtcgtcactaccacccagtcgagaaggaaaggctcggtgaactgcgttgttatcgaccaagtacttgagctggccaaagacgcgagagcggtgctctgtatgagtcagaccctccaccttctgaactacctctcgtcccgcaaggtcccgggcgtggagacggatatcagcccatcggcccagcagctgctctcccagcgcagccacatctactcggggctcgacggccaccttagcaccgtctgcggcggccgtagttgagccaggggatgcgggggatgaggctctgtcaactacttcggtcatggcgtcgttgcttgtgtgatttttgaggacccatccctttggtatataagtatactctggggttaaggttgcccgtgtagtctaggttatagttttcatgtgaaataccgagagccgagggagaataaacgggggtatttggacttgtttttttcgcggaaaagcgtcgaatcaaccctgcgggccttgcaccatgtccacgacgtgtttctcgccccaattcgccccttgcacgtcaaaattaggcctccatctagacccctccataacatgtgactgtggggaaaagtataagggaaaccatgcaaccatagacgacgtgaaagacggggaggaaccaatggaggccaaagaaatggggtagcaacagtccaggagacagacaaggagacaaggagagggcgcccgaaagatcggaaaaacaaacatgtccaattggggcagtgacggaaacgacacggacacttcagtacaatggaccgaccatctccaagccagggttattccggtatcaccttggccgtaacctcccgctggtacctgatattgtacacgttcacattcaatatactttcagctacaataagagaggctgtttgtcgggcatgtgtgtccgtcgtatggggtgatgtccgagggcgaaattcgctacaagcttaactctggcgcttgtccagtatgaatagacaagtcaagaccagtggtgccatgattgacagggaggtacaagacttcgatactcaagcattactcggacttgtggcgattgaacagacgggcgatcgcttctcccccgtattgccccgtttctacgagtgccgtacgagtacgagtacaggaggtacagtacattccgatacttgcactcccaccattgctgtatatctctgctacttgtggtatcaaatccgctactcgtagtggtttttccacttgtacaaattctatcccccatgaccaccaacccttctcccgatcgccagtctcacttggcggaatcgtcgacgatcatcctcgagatgcggccgcgtcgacataacttcgtatagcatacattatacgaagttattttctaatttggaccgatagccgtatagtccagtctatctataagttcaactaactcgtaactattaccataacatatacttcactgccccagataaggttccgataaaaagttgtgcagactaaatttatttcagtctcctcttcaccaccaaaatgccctcctacgaagcgcgagctaacgtccacaagtccgcctttgccgcccgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggaactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggatgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagatgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtcatttaactgtgtatatagagagcgtgcaagtatggagcgcttgttcagcttgtatgatggtcagacgacctgtctgatcgagtatgtatgatactgcacaacctgataacttcgtatagcatacattatacgaagttatctcgaggcggccgcatggagcgtgtgttctgagtcgatgttttctatggagttgtgagtgttagtagacatgatgggtttatatatgatgaatgaatagatgtgattttgatttgcacgatggaattgagaactttgtaaacgtacatgggaatgtatgaatgtgggggttttgtgactggataactgacggtcagtggacgccgttgttcaaatatccaagagatgcgagaaactttgggtcaagtgaacatgtcctctctgttcaagtaaaccatcaactatgggtagtatatttagtaaggacaagagttgagattctttggagtcctagaaacgtattttcgcgttccaagatcaaattagtagagtaatacgggcacgggaatccattcatagtctcaattttcccataggtgtgctacaaggtgttgagatgtggtacagtaccaccatgattcgaggtaaagagcccagaagtcattgatgaggtcaagaaatacacagatctacagctcaatacaatgaatatcttctttcatattcttcaggtgacaccaagggtgtctattttccccagaaatgcgtgaaaaggcgcgtgtgtagcgtggagtatgggttcggttggcgtatccttcatatatcgacgaaatagtagggcaagagatgacaaaaagtatctatatgtagacagcgtagaatatggatttgattggtataaattcatttattgcgtgtctcacaaatactctcgataagttggggttaaactggagatggaacaatgtcgatatctcgacatattttgatatttgttaattaa

SEQ ID NO: 177 - DNA, 5655 bp, synthetic construct
  misc_feature 1..5655: note = pLD137
  source 1..5655: mol_type = other DNA
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctgatcccaatattacacccaagtagcatgcataagctaaaagtaactcgcagcgcacaccgtgcagattcataagtctatgattaattgaacgccaataacccggcttactacaagtacaagtaggtatacatagcggtaatgaatcattagaaaaataaaaaacaaaaaaaaacaaaacaaactgttgtggatgcatcaacagtagtacatagttgtacgatgtacttgtacttgtaaaagcaaaaatgtacaatatctcagggagcgcaacttttacgttcgaagaacaatgtaccgcataccgcattctagattctgcggaacgtctaacctggaaatacgattttttatttctttcattttttttgcttcttcaaaagtatggtaatttcctaccattacagttgacactgaacgaggggggattgaatttaagcaaaaaattaaatcaaaatacctttatgtatccagcccatgtaataaacaaaaggattatataacaagaaataaatatatacctttaatggatcattagaataaaaataaatacgagaagcacaccagagaagctttttgattgccactataccgctactttggtatatcttattataattgttgaatttgcaagatagaatgtcattcattggagagaaatccaaggaatatgtgggatgaaatgactagaagtatgaacaatgagaatagtacatacttgtacctgtatttctagaagagagaaagacagttgagtgtgtgattctcgtccaataataatctcaatagtaacgtgtgaatagctgttctttgatagttgatatttctcgatgactatttatgttgtacaagggatttttttcgttgctgttgatttcgaattaggcaatgcagatatcatttatgctatccatatttaagatttcccatacgcatttataacatttattctacataaattgttaaatgaacgaactgccattataaattgtttcctaaataggaagtgtttttcataaagcaagtaagttgtctaataatactaagtaataaaaataagttcatacaatatattttgagaacatcatttggaggcggtagatggagtctgtttattattaaacaatgcgagatgaccccttaaatattgagaacatcagttggaggcggcagatggagtctgtctatttagcaatgggacatgactgtcagtatcatcatatgtatatatataatacatataatattatataacacgatttttttaaattattggcccgaaaattaatcagtgtagactggatcctcgagaaccatttaattaagatcagagaccggatggtctcaagcgtcgacaccatatcatataaaactaacaatgcattgaattaatacgaagactacccgttgctatctccacaccgttatctccacggtccaaaggctgctcaatgtgctgcatacgtaacgtggggtgcaaccttgagcacatagtactttccgaaaaccggcgataattaagtgtgcactccaacttttcacactgagcataaaatgtggagaagaaatcacactaaaaagtcaggtagactggaaaatgcgccatgaaataatatctcttgctacagtaatgcccagcatcgaggggtattgtgtcaccaacactatagtggcagctgaagcgctcgtgattgtagtatgagtctttattggtgatgggaagagttcactcaatattctcgttactgccaaaacaccacggtaatcggccagacaccatggatgtagatcaccaagcctgtgaatgttattcgagctaaaatgcacatggttggtgaaaggagtagttgctgtcgaattccgtcgtcgcctgagtcatcatttatttaccagttggccacaaacccttgacgatctcgtatgtcccctccgacatactcccggccggctggggtacgttcgatagcgctatcggcatcgacaaggtttgggtccctagccgataccgcactacctgagtcacaatcttcggaggtttagtcttccacatagcacgggcaaaagtgcgtatatatacaagagcgtttgccagccacagattttcactccacacaccacatcacacatacaaccacacacatccacaatggaacccgaaactaagaagaccaagactgactccaagaagattgttcttctcggcggcgacttctgtggccccgaggtgattgccgaggccgtcaaggtgctcaagtctgttgctgaggcctccggcaccgagtttgtgtttgaggaccgactcattggaggagctgccattgagaaggagggcgagcccatcaccgacgctactctcgacatctgccgaaaggctgactctattatgctcggtgctgtcggaggcgctgccaacaccgtatggaccactcccgacggacgaaccgacgtgcgacccgagcagggtcttctcaagctgcgaaaggacctgaacctgtacgccaacctgcgaccctgccagctgctgtcgcccaagctcgccgatctctcccccatccgaaacgttgagggcaccgacttcatcattgtccgagagctcgtcggaggtatctactttggagagcgaaaggaggatgacggatctggcgtcgcttccgacactgagacttactccgttcctgaggttgagcgaattgcccgaatggccgccttcctggcccttcagcacaacccccctcttcccgtgtggtcccttgacaaggccaacgtgctggcctcctctcgactttggcgaaagaccgtcacccgagtcctcaaggacgaattcccccagctggagctcaaccaccagctgatcgactcggccgccatgatcctcatcaagcagccctccaagatgaatggtatcatcatcaccaccaacatgtttggcgatatcatctccgacgaggcctccgtcatccccggttctctgggtctgctgccctccgcctctctggcttctctgcccgacaccaacgaggcgttcggtctgtacgagccctgtcacggatctgcccccgatctcggcaagcagaaggtcaaccccattgccaccattctgtctgccgccatgatgctcaagttctctcttaacatgaagcccgccggtgacgctgttgaggctgccgtcaaggagtccgtcgaggctggtatcactaccgccgatatcggaggctcctcctccacctccgaggtcggagactttgttgccaacaaggtcaaggagctgctcaagaaggagtaagtcgtttctacgacgcattgatggaaggagcaaactgacgcgcctgcgggttggtctaccggcagggtccgctagtgtataagactctataaaaagggcccagccctgctaatgaaatgatgatttataatttaccggtgtagcaaccttgactagaagaagcagattgggtgtgtttgtagtggaggacagtggtacgttttaattaa

SEQ ID NO: 178 - DNA, 4664 bp, synthetic construct
  misc_feature 1..4664: note = pLD138
  source 1..4664: mol_type = other DNA
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacctgatcccaatattacacccaagtagcatgcataagctaaaagtaactcgcagcgcacaccgtgcagattcataagtctatgattaattgaacgccaataacccggcttactacaagtacaagtaggtatacatagcggtaatgaatcattagaaaaataaaaaacaaaaaaaaacaaaacaaactgttgtggatgcatcaacagtagtacatagttgtacgatgtacttgtacttgtaaaagcaaaaatgtacaatatctcagggagcgcaacttttacgttcgaagaacaatgtaccgcataccgcattctagattctgcggaacgtctaacctggaaatacgattttttatttctttcattttttttgcttcttcaaaagtatggtaatttcctaccattacagttgacactgaacgaggggggattgaatttaagcaaaaaattaaatcaaaatacctttatgtatccagcccatgtaataaacaaaaggattatataacaagaaataaatatatacctttaatggatcattagaataaaaataaatacgagaagcacaccagagaagctttttgattgccactataccgctactttggtatatcttattataattgttgaatttgcaagatagaatgtcattcattggagagaaatccaaggaatatgtgggatgaaatgactagaagtatgaacaatgagaatagtacatacttgtacctgtatttctagaagagagaaagacagttgagtgtgtgattctcgtccaataataatctcaatagtaacgtgtgaatagctgttctttgatagttgatatttctcgatgactatttatgttgtacaagggatttttttcgttgctgttgatttcgaattaggcaatgcagatatcatttatgctatccatatttaagatttcccatacgcatttataacatttattctacataaattgttaaatgaacgaactgccattataaattgtttcctaaataggaagtgtttttcataaagcaagtaagttgtctaataatactaagtaataaaaataagttcatacaatatattttgagaacatcatttggaggcggtagatggagtctgtttattattaaacaatgcgagatgaccccttaaatattgagaacatcagttggaggcggcagatggagtctgtctatttagcaatgggacatgactgtcagtatcatcatatgtatatatataatacatataatattatataacacgatttttttaaattattggcccgaaaattaatcagtgtagactggatcctcgagaaccatttaattaatttctaatttggaccgatagccgtatagtccagtctatctataagttcaactaactcgtaactattaccataacatatacttcactgccccagataaggttccgataaaaagttgtgcagactaaatttatttcagtctcctcttcaccaccaaaatgccctcctacgaagcgcgagctaacgtccacaagtccgcctttgccgcccgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggaactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggatgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagatgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtcatttaactgtgtatatagagagcgtgcaagtatggagcgcttgttcagcttgtatgatggtcagacgacctgtctgatcgagtatgtatgatactgcacaacctgcgcttgagaccatccggtctctgatcttaattaa

SEQ ID NO: 179 - DNA, 5975 bp, synthetic construct
  misc_feature 1..5975: note = pLD139
  source 1..5975: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgaacccaagggaaaacttccttaagtgttttctgcagtacatccctaacaatgcaacaaacctcaagttggtgtacactcaaaacaatccactctatatgagcgtgcttaatagcacaatccacaacttgcgcttcacgtcagatactacgcctaagccactagtgatcgttacaccatcacacgtcagccatattcaaggaacgatcctatgtctgaaaaaggtcgggttgcaaatcaggactcgatcaggagggcacgatagtgagggaatgagttacatctcgcaagtacccttcgtgatagttgacttgcgaaatatgcggtctattaaaattgacgtacatagccagaccgcctgggttgaagcaggggcaaccttgggtgaagtttattactgggtcaatgaaaaaaacgaaaacctaagtcttgctgctggatattgccccaccgtttgcgcgggtggtcattttggaggcggcggatatggtccgttgatgagaaattatggacttgcagcagacaatattatagatgcccacttggtgaacgttcatggaaaggtcttggaccgtaagtccatgggtgaagatcttttctgggccttgagaggtggtggagcggaatcgtttggcatcatcgttgcctggaaaattaggttggttgcggtcccgaagagtacaatgttctccgtgaagaagattatggaaatacatgagcttgtcaagttagttaacaagtggcaaaatatcgcttataagtatgataaagacttgcttttgatgactcattttattacgcgaaacataaccgataaccagggcaagaacaagactgctattcacacgtacttctcctctgtatttcttggaggagtagactccttagttgacttgatgaacaagagtttcccagaattggggattaagaagacagattgcagacaattatcgtggatagatacaatcatattctatagcggtgtcgtcaattacgatactgataattttaataaagaaatcctcctagatcgttcagctgggcaaaacggggcattcaaaattaaattggattatgtgaagaaaccaattccagagctggtgtttgttcagatattggaaaaactttacgaagaagacattggcgcaggtatgtacgctttgtatccatatggaggcattatggacgagatctcagagctggcgatccccttcccgcacagagctgggatactctacgagctatggtacatctgctcttgggagaaacaagaagacaacgagaaacatctcaattggattcggaacatatacaactttatgaccccatacgtatcaaaaaacccgcgcttagcatacttgaattacagagacttagatatcggtatcaatgatcctaagaatcctaacaattacacccaagcccgtatttggggtgagaaatatttcggcaagaattttgacagattagttaaggtcaaaacactcgtggaccccaacaactttttccgaaacgagcagtcgattccaccactacccaggcatagacacggaagaagggcaaagttgtaagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 180 - DNA, 5594 bp, synthetic construct
  misc_feature 1..5594: note = pLD19
  source 1..5594: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatgggtttatcgtcagtgtgcactttttcttttcaaacaaactaccacaccctcctaaaccctcacaataataacccaaaaacctccttgctatgttacagacatccaaagacaccgatcaagtattcatacaacaattttcccagtaaacattgctcaacgaagtccttccacttgcaaaacaaatgcagcgaatcattgtcgatagctaaaaactcgatacgtgcggcaaccactaaccaaactgagccaccagagagcgataatcattcagtcgccaccaagattttgaactttggaaaagcctgttggaaacttcaaaggccttacaccattatcgcatttaccagttgcgcatgtggtttgttcgggaaggaattattacacaacacaaatttgatcagctggagcctaatgtttaaggcatttttcttcttagttgcaattttgtgtatagcttcgtttacaacgaccattaatcagatttacgaccttcacatcgatcggatcaataaaccagacttgccccttgcctctggggaaatctctgtaaatactgcatggatcatgctgataatcgtggctttgtttggattgattattacaattaagatgaaggggggtccattatatatattcgggtactgcttcggcattttcggtggtatcgtttactccgtcccaccctttagatggaaacagaaccccagtacggcctttctactcaatttcttggctcatatcatcacaaacttcacattctattatgcaagccgagcggcgcttggtttgccgttcgaactcagaccgagttttacatttctccttgccttcatgaaactgatgggactggcccttgcattgatcaaggatgcgtcagatgtcgaaggcgacactaagttcggcattctgacgcttgcttccaagtatggaagtagaaatctaacgcttttttgttcaggaatagtgctacttagttatgttgctgctatactcgctggcattatttggcctcaggccttcaactctaacgtaatgttgttatcccatgctattttggcgttctggttgatcttgcaaacgcgagattttgcactcactaactacgacccagaggcaggaaggcgcttttacgagtttatgtggaagttgtattatgccgaatacttggtttatgttttcatttgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 181 - DNA, 5372 bp, synthetic construct
  misc_feature 1..5372: note = pLD26
  source 1..5372: mol_type = other DNA
ttaattaattccgcttaatggagtccaaaaagaccaacctctgcgcctcgatcgacgtgaccacaaccgccgagttcctttcgctcatcgacaagctcggtccccacatctgtctcgtgaagacgcacatcgatatcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgttggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctagttcggcatggcagatcatcatgcctgcaggagctccaattgtaatatttcgggagaaatatcgttggggtaaaacaacagagagagagagggagagatggttctggtagaattataatctggttgttgcaaatgctactgatcgactctggcaatgtctgtagctcgctagttgtatgcaacttaggtgttatgcatacacacggttattcggttgaattgtggagtaaaaattgtctgagttgtgtcttagctactggctggccccccgcgaaagataatcaaaattacacttgtgaatttttgcacacacaccgattaacatttcccttttttgtccaccgatacacgcttgcctcttcttattttctctgtgcttccccctcctgtgactttttccaccattgatataaaatcaactccatttccctaaaatctccccagattctaaaaacaacttcttctcttctgcttttccttatttttgttatatttatttaccatcccttattttgaatagttattccccactaacattgttcaaatcttcacgacataatggcggcaaccactaaccaaactgagccaccagagagcgataatcattcagtcgccaccaagattttgaactttggaaaagcctgttggaaacttcaaaggccttacaccattatcgcatttaccagttgcgcatgtggtttgttcgggaaggaattattacacaacacaaatttgatcagctggagcctaatgtttaaggcatttttcttcttagttgcaattttgtgtatagcttcgtttacaacgaccattaatcagatttacgaccttcacatcgatcggatcaataaaccagacttgccccttgcctctggggaaatctctgtaaatactgcatggatcatgctgataatcgtggctttgtttggattgattattacaattaagatgaaggggggtccattatatatattcgggtactgcttcggcattttcggtggtatcgtttactccgtcccaccctttagatggaaacagaaccccagtacggcctttctactcaatttcttggctcatatcatcacaaacttcacattctattatgcaagccgagcggcgcttggtttgccgttcgaactcagaccgagttttacatttctccttgccttcatgaaactgatgggactggcccttgcattgatcaaggatgcgtcagatgtcgaaggcgacactaagttcggcattctgacgcttgcttccaagtatggaagtagaaatctaacgcttttttgttcaggaatagtgctacttagttatgttgctgctatactcgctggcattatttggcctcaggccttcaactctaacgtaatgttgttatcccatgctattttggcgttctggttgatcttgcaaacgcgagattttgcactcactaactacgacccagaggcaggaaggcgcttttacgagtttatgtggaagttgtattatgccgaatacttggtttatgttttcatttgataagagtgactcttttgataagagtcgcaaatttgatttcataagtatatattcattatgtaaagtagtaaatggaaaattcattaaaaaaaaagcaaatttccgttgtatgcatactccgaacacaaaactagccccggaaaaacccttagttgatagttgcgaatttaggtcgaccatatgcgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaataaaaaagtgaaaaataaaatcaaacccaaagatcaacctccccatcatcatcgtcatcaaacccccagctcaattcgcaatggttagcacaaaaacatacacagaaagggcatcagcacacccctccaaggttgcccaacgtttattaattaaaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 182 - protein, 11 aa, synthetic construct
  REGION 1..11: note = VSV-G
YTDIEMNRLGK

(19) United States
(12) Patent Application Publication
(10) Pub. No.: US 2024/0384314 A1
(43) Pub. Date: Nov. 21, 2024

(54) PRODUCTION OF CANNABINOIDS IN YEAST USING A FATTY ACID FEEDSTOCK

(71) Applicant: Pyrone Systems, Inc., San Diego, CA (US)

(72) Inventors: Alexander Hutagalung, San Diego, CA (US); Jose Miguel Laplaza, San Diego, CA (US)

(21) Appl. No.: 18/421,170

(22) Filed: Jan. 24, 2024

Related U.S. Application Data
(63) Continuation of application No. 17/449,847, filed on Oct. 4, 2021, now Pat. No. 11,884,948.
Application No. 17/449,847 is a continuation of application No. 16/783,122, filed on Feb. 5, 2020, now Pat. No. 11,136,605.
Application No. 16/783,122 is a continuation of application No. PCT/US2019/051357, filed on Sep. 16, 2019.
(60) Provisional application No. 62/731,978, filed on Sep. 17, 2018.
(60) Provisional application No. 62/731,980, filed on Sep. 17, 2018.

(51) Int. Cl. C12P 17/06; C12N 1/16; C12P 7/42

(52) U.S. Cl. CPC C12P 17/06; C12N 1/16; C12P 7/42; C12Y 103/03006; C12Y 203/01206; C12Y 404/01026

(57) ABSTRACT

Strains of yeasts are provided containing the genes for the production of cannabinoids from fatty acids. The enzymes that mediate cannabinoid production are localized to the cytosol, peroxisome or different compartments within the secretory pathway (e.g., endoplasmic reticulum, Golgi, vacuole) to ensure efficient production. The engineered microorganisms produce cannabinoids in a controlled fermentation process.

### INCORPORATION BY REFERENCE TO RELATED APPLICATIONS

Any and all priority claims identified in the Application Data Sheet, or any correction thereto, are hereby incorporated by reference under 37 CFR 1.57. This application is a continuation of U.S. patent application Ser. No. 17/449,847, filed on Oct. 4, 2021, which is a continuation of U.S. patent application Ser. No. 16/783,122, filed Feb. 5, 2020, which issued as U.S. Pat. No. 11,136,605 on Oct. 5, 2021, which is a continuation of PCT International Application No. PCT/US2019/051357, filed Sep. 16, 2019, designating the United States and published in English, which claims the benefit of U.S. Provisional Application No. 62/731,978, filed Sep. 17, 2018, and U.S. Provisional Application No. 62/731,980, filed Sep. 17, 2018. Each of the aforementioned applications is incorporated by reference herein in its entirety, and each is hereby expressly made a part of this specification.

### FIELD

Strains of yeasts are provided containing the genes for the production of cannabinoids from fatty acids. The enzymes that mediate cannabinoid production are localized to the cytosol, peroxisome, or different compartments within the secretory pathway (e.g., endoplasmic reticulum, Golgi, vacuole) to ensure efficient production. The engineered microorganisms produce cannabinoids in a controlled fermentation process.

### REFERENCE TO SEQUENCE LISTING

This application is filed with an electronic sequence listing entitled SEQLISTING_PYSYS002C3.xml, created on Jan. 24, 2024, which is 421 KB in size. The information in the electronic sequence listing is hereby incorporated by reference in its entirety.

### BACKGROUND

Microorganisms employ various enzyme-driven biological pathways to support their own metabolism and growth. A cell synthesizes native proteins, including enzymes, in vivo from deoxyribonucleic acid (DNA). DNA first is transcribed into a complementary ribonucleic acid (RNA) that comprises a ribonucleotide sequence encoding the protein. RNA then directs translation of the encoded protein by interaction with various cellular components, such as ribosomes. The resulting enzymes participate as biological catalysts in pathways involved in production of molecules by the organism.

### SUMMARY

These pathways can be exploited for the harvesting of the naturally produced products. The pathways also can be altered to increase production or to produce different products that may be commercially valuable. Advances in recombinant molecular biology methodology allow researchers to isolate DNA from one organism and insert it into another organism, thus altering the cellular synthesis of enzymes or other proteins. Advances in recombinant molecular biology methodology also allow endogenous genes, carried in the genomic DNA of a microorganism, to be increased in copy number, thus altering the cellular synthesis of enzymes or other proteins. Such genetic engineering can change the biological pathways within the host organism, causing it to produce a desired product.

Microorganic industrial production instead of plant production can increase the availability of natural products while reducing the manufacturing and environmental cost.

### BRIEF DESCRIPTION OF THE DRAWINGS

FIG. 1 depicts production of cannabinoids by producing hexanoic acid in the peroxisome. A fatty acid (FA) enters the peroxisome and is activated by a fatty acyl-CoA synthetase/fatty acyl activating enzyme (FAA) to become a fatty acyl-CoA (FA-CoA). It undergoes multiple rounds of beta oxidation, with each round producing one acetyl-CoA. Eventually, a molecule of hexanoyl-CoA is produced. An acyl-CoA thioesterase (TES) acts on the hexanoyl-CoA to produce hexanoic acid (hexanoate). The hexanoic acid leaves the peroxisome and is activated by a hexanoyl-CoA synthetase (HXS) to become hexanoyl-CoA, which is then acted upon by a polyketide synthase (TKS) and olivetolic acid cyclase (OAC) to produce olivetolic acid. A prenyltransferase (PTS) adds a geranyl moiety to olivetolic acid to produce cannabigerolic acid, which is converted to one of several cannabinoids as described in FIG. 4.

FIG. 2 depicts production of cannabinoids by producing olivetolic acid in the peroxisome. A fatty acid (FA) enters the peroxisome and is activated by a fatty acyl-CoA synthetase/fatty acyl activating enzyme (FAA) to become a fatty acyl-CoA (FA-CoA). It undergoes multiple rounds of beta oxidation, with each round producing one acetyl-CoA. Eventually, a molecule of hexanoyl-CoA is produced, which is then acted upon by a polyketide synthase (TKS) and olivetolic acid cyclase (OAC) to produce olivetolic acid. If necessary, an acetyl-CoA carboxylase (ACC) will be localized to the peroxisome to produce additional malonyl-CoA. The olivetolic acid leaves the peroxisome and enters the cytosol. A prenyltransferase (PTS) adds a geranyl moiety to olivetolic acid to produce cannabigerolic acid, which is converted to one of several cannabinoids as described in FIG. 4.

FIG. 3 depicts production of cannabinoids by producing cannabigerolic acid in the peroxisome. A fatty acid (FA) enters the peroxisome and is activated by a fatty acyl-CoA synthetase/fatty acyl activating enzyme (FAA) to become a fatty acyl-CoA (FA-CoA). It undergoes multiple rounds of beta oxidation, with each round producing one acetyl-CoA. Eventually, a molecule of hexanoyl-CoA is produced, which is then acted upon by a polyketide synthase (TKS) and olivetolic acid cyclase (OAC) to produce olivetolic acid. If necessary, an acetyl-CoA carboxylase (ACC) will be localized to the peroxisome to produce additional malonyl-CoA. A prenyltransferase (PTS) adds a geranyl moiety to olivetolic acid to produce cannabigerolic acid, which is converted to one of several cannabinoids as described in FIG. 4.

FIG. 4 depicts compartmentalization of enzymes for cannabinoid production. Cannabidiolic acid (CBDA) synthase, tetrahydrocannabidiolic acid (THCA) synthase or cannabichromenic acid (CBCA) synthase will be localized to: A) the cytoplasm, B) the peroxisome, C) compartments of the secretory pathway, such as the endoplasmic reticulum (ER) or Golgi or D) the vacuole. The enzymes will be localized to different compartments through the use of signal sequences.

FIG. 5 provides a map of plasmid pLD1.

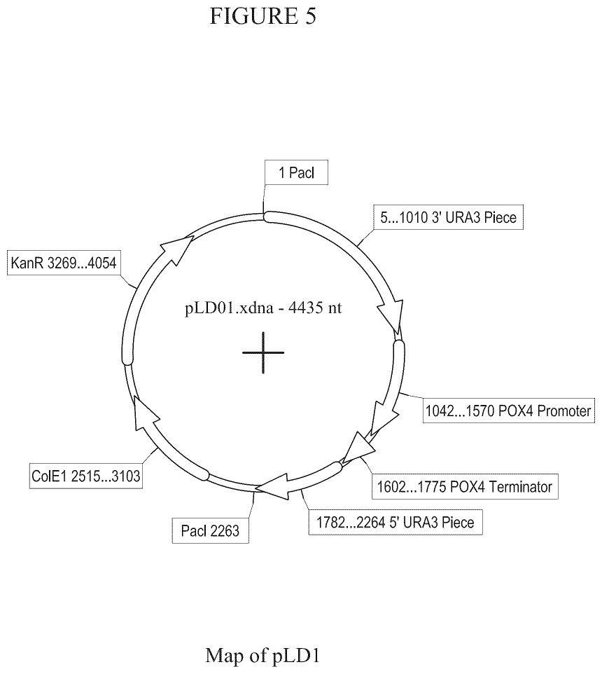

FIG. 6 provides a map of plasmid pLD10.

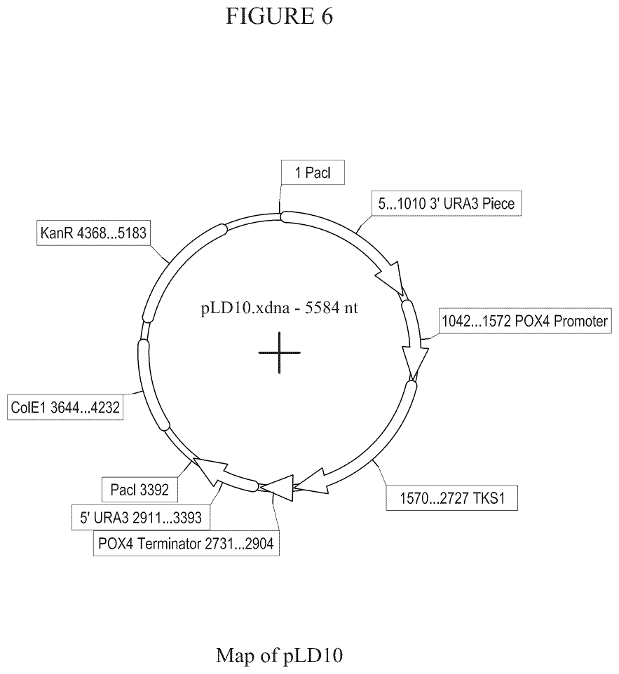

FIG. 7 provides a map of plasmid pLD12.

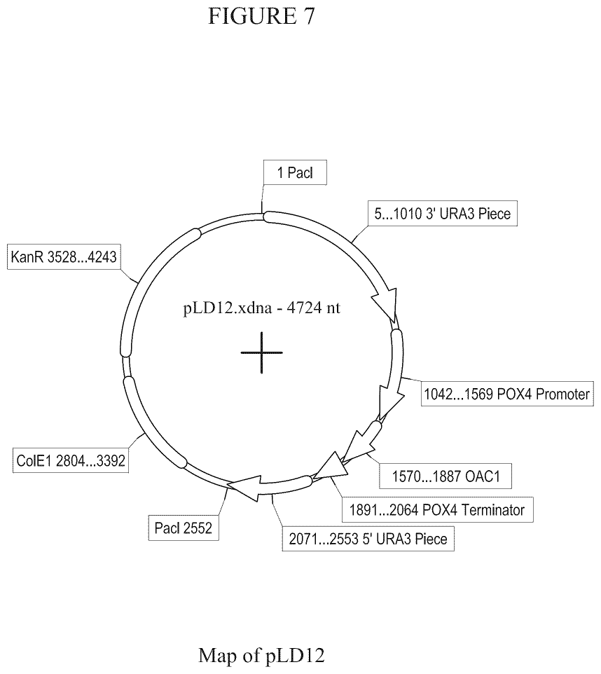

FIG. 8 provides a map of plasmid pLD14.

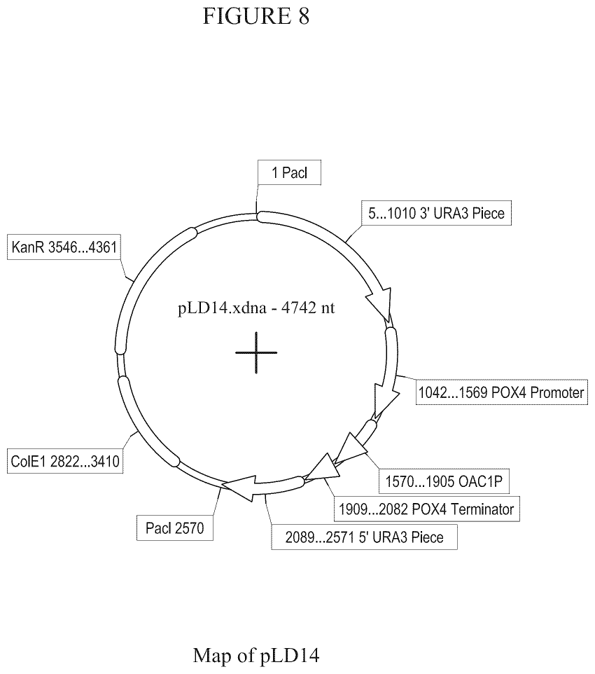

FIG. 9 provides a map of plasmid pLD16.

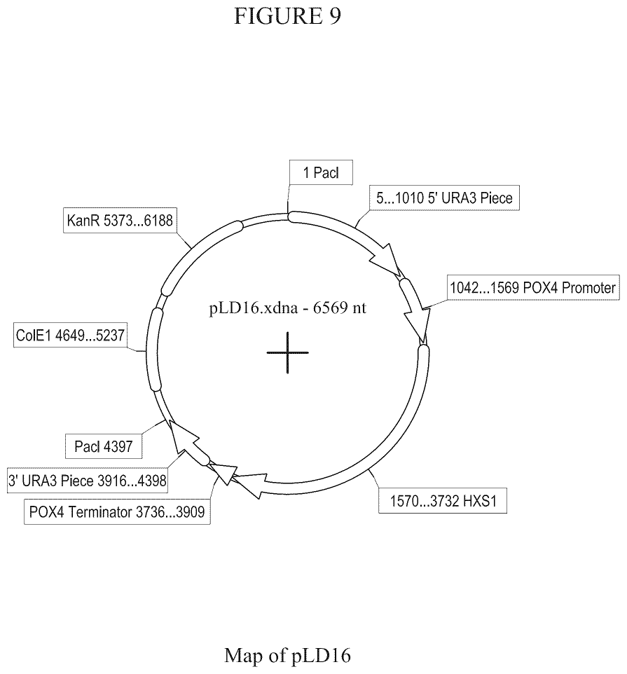

FIG. 10 provides a map of plasmid pLD20.

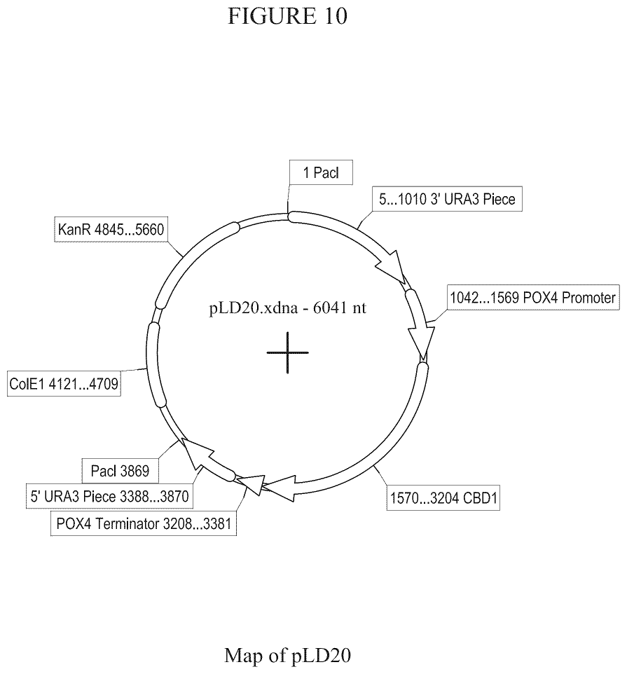

FIG. 11 provides a map of plasmid pLD22.

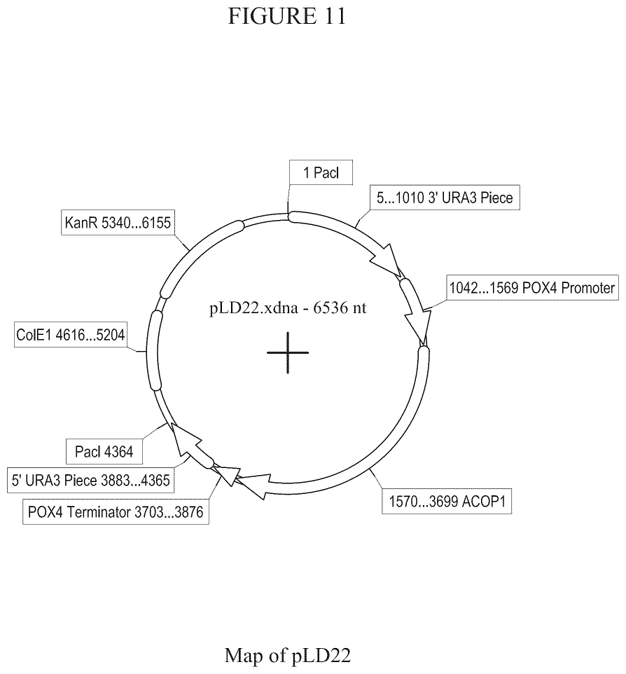

FIG. 12 provides a map of plasmid pLD24.

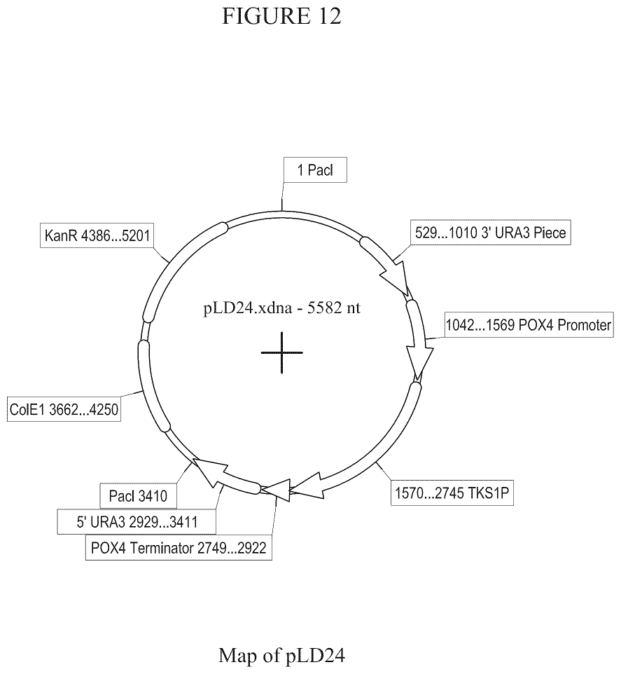

FIG. 13 provides a map of plasmid pLD56.

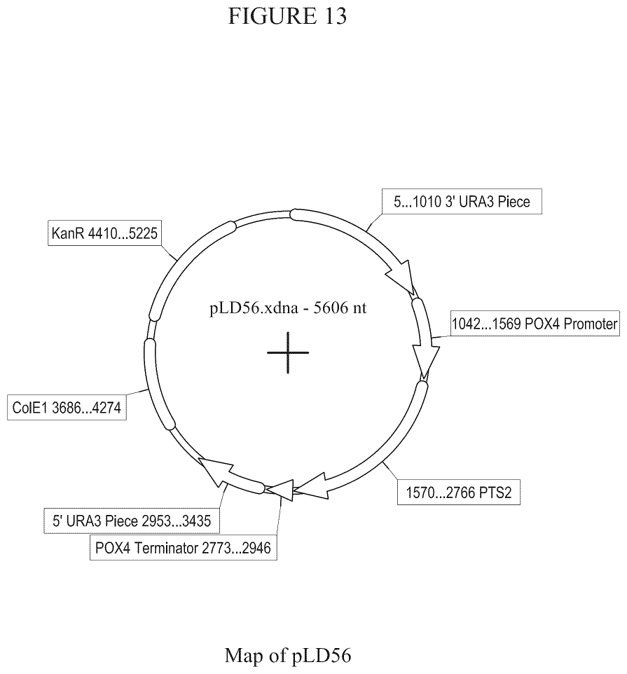

FIG. 14 provides a map of plasmid pLD87.

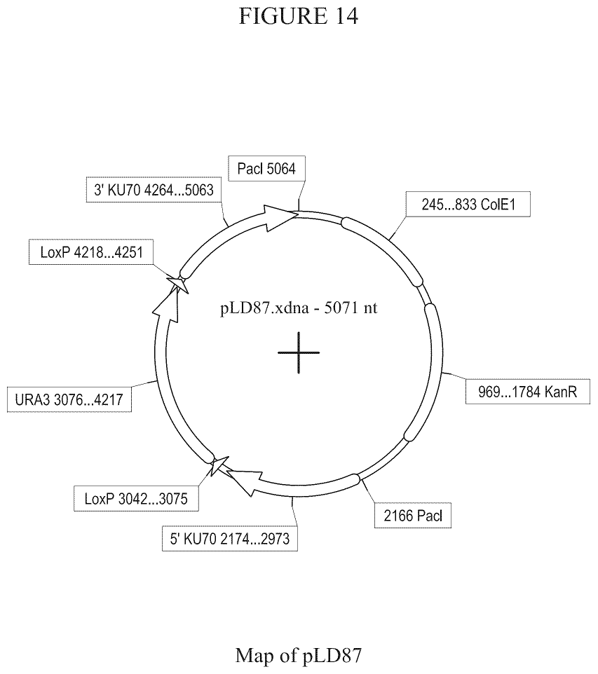

FIG. 15 provides a map of plasmid pLD101.

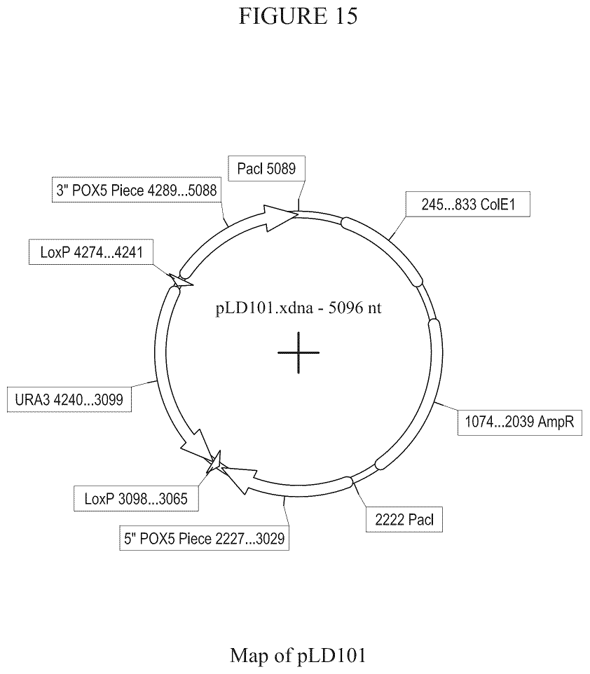

FIG. 16 provides a map of plasmid pLD102.

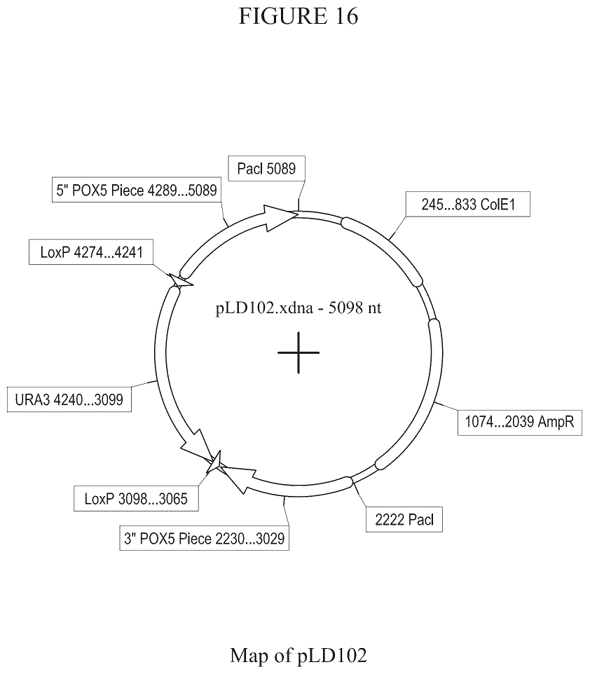

FIG. 17 provides a map of plasmid pLD111.

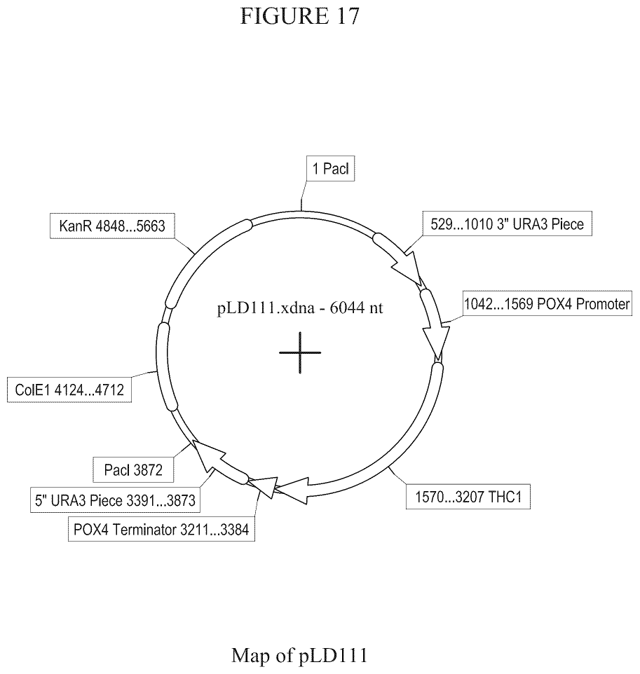

FIG. 18 provides a map of plasmid pLD112.

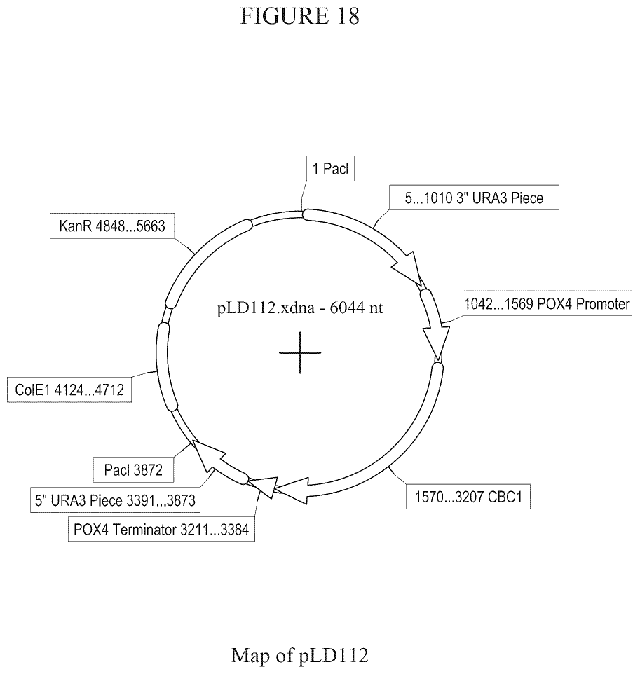

FIG. 19 provides a map of plasmid pLD113.

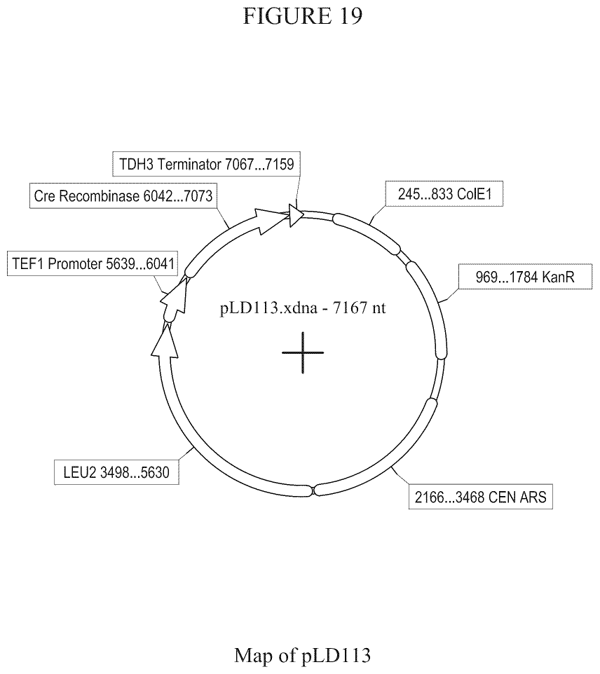

FIG. 20 provides a map of plasmid pLD125.

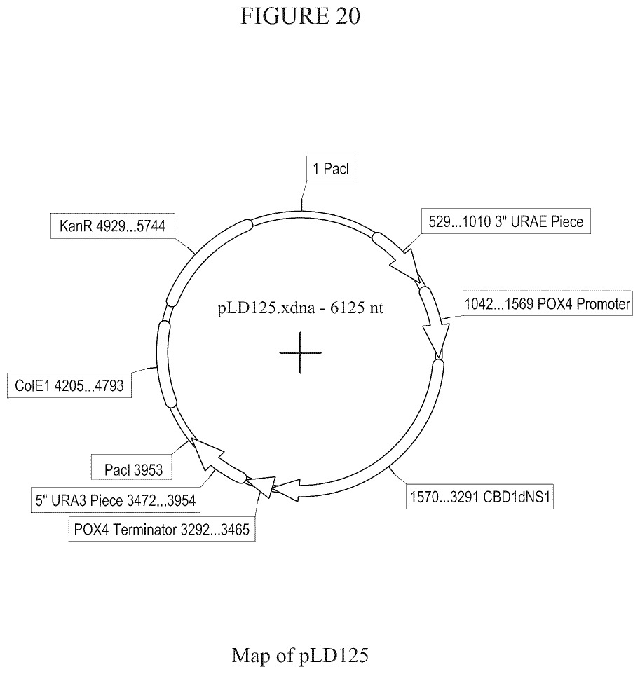

FIG. 21 provides a map of plasmid pLD127.

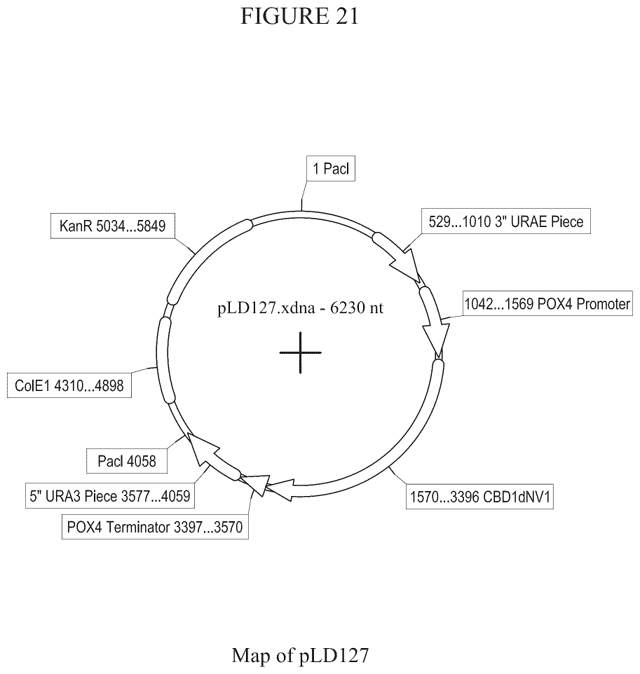

FIG. 22 provides a map of plasmid pLD131.

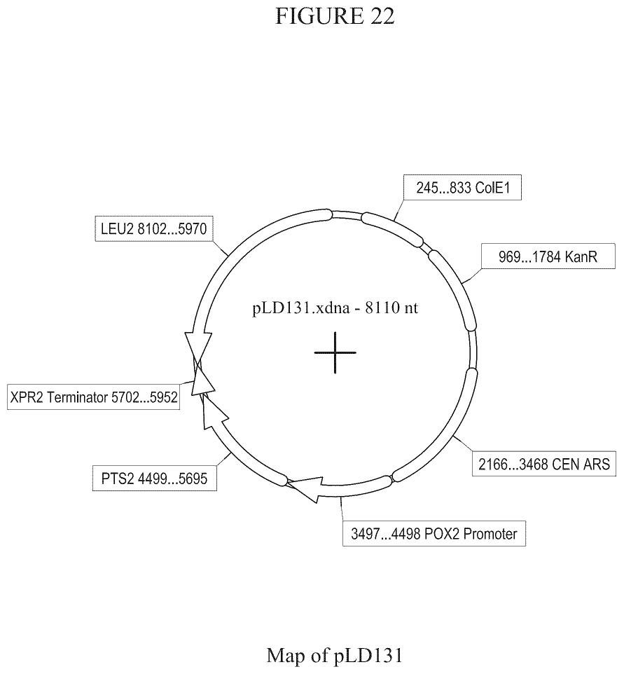

FIG. 23 provides a map of plasmid pLD132.

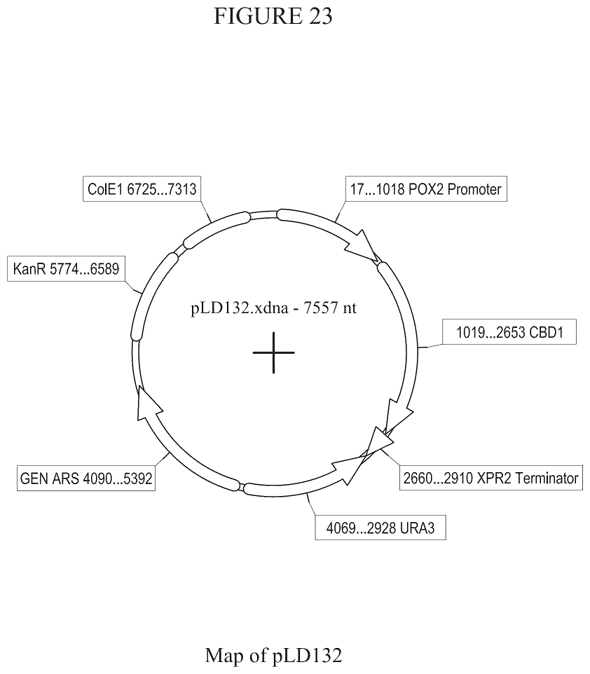

FIG. 24 provides a map of plasmid pLD135.

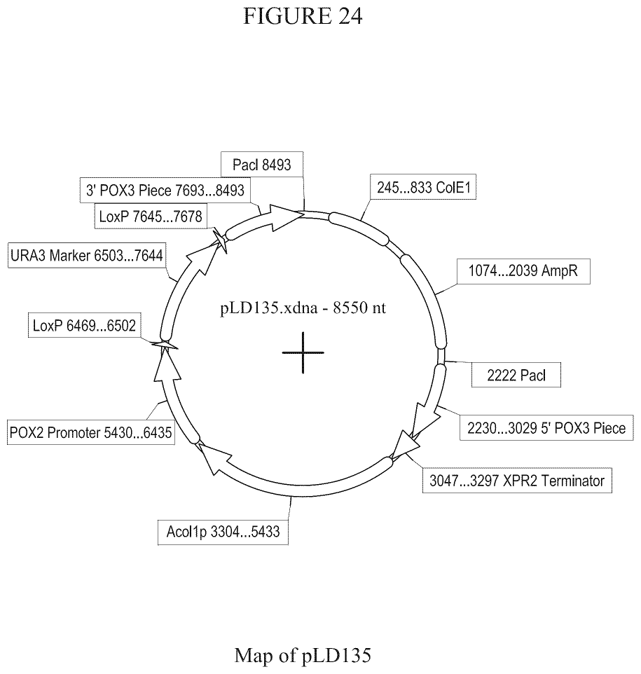

FIG. 25 provides a map of plasmid pLD137.

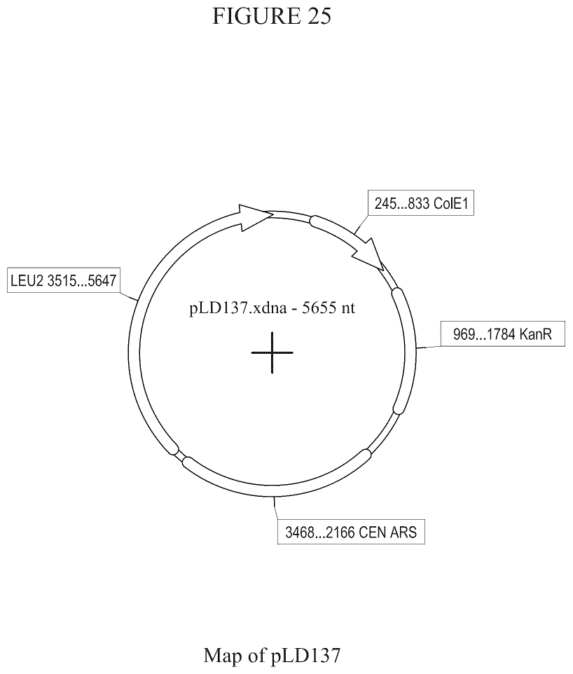

FIG. 26 provides a map of plasmid pLD138.

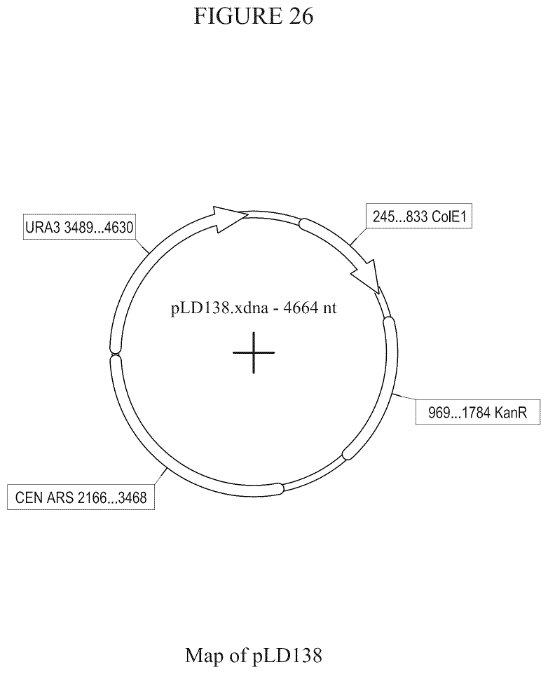

FIG. 27 provides a map of plasmid pLD139.

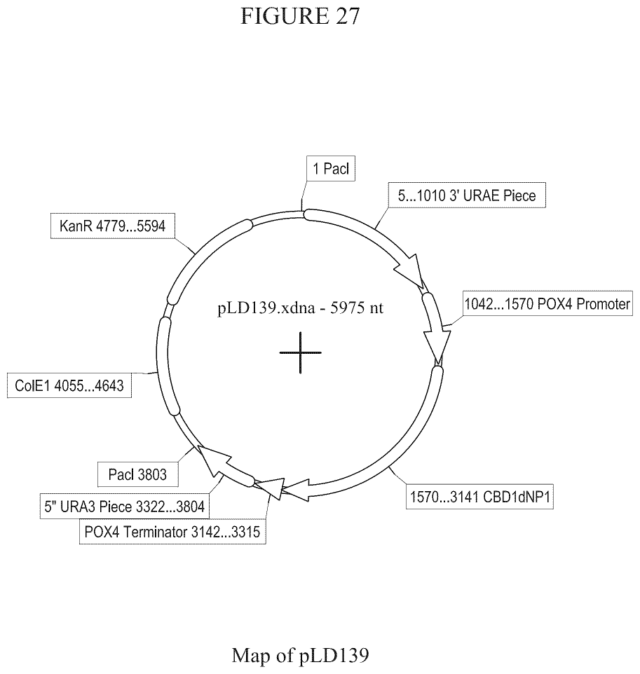

FIG. 28 provides a map of plasmid pLD19.

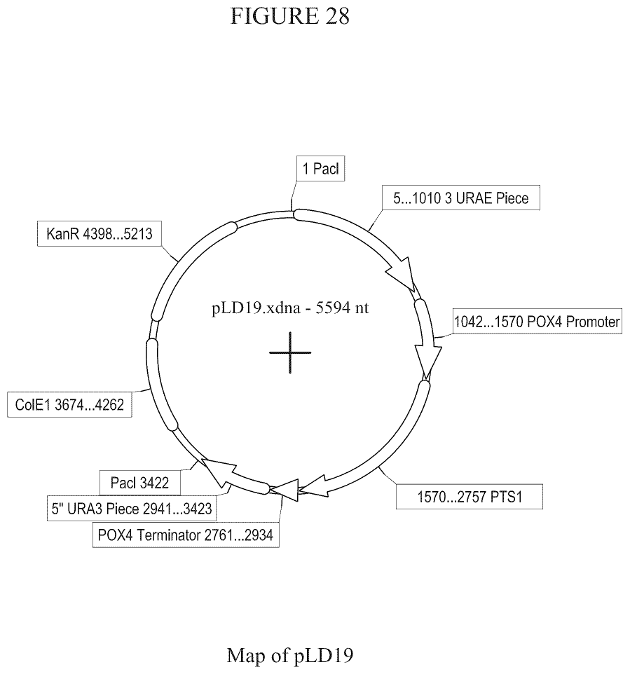

FIG. 29 provides a map of plasmid pLD26.

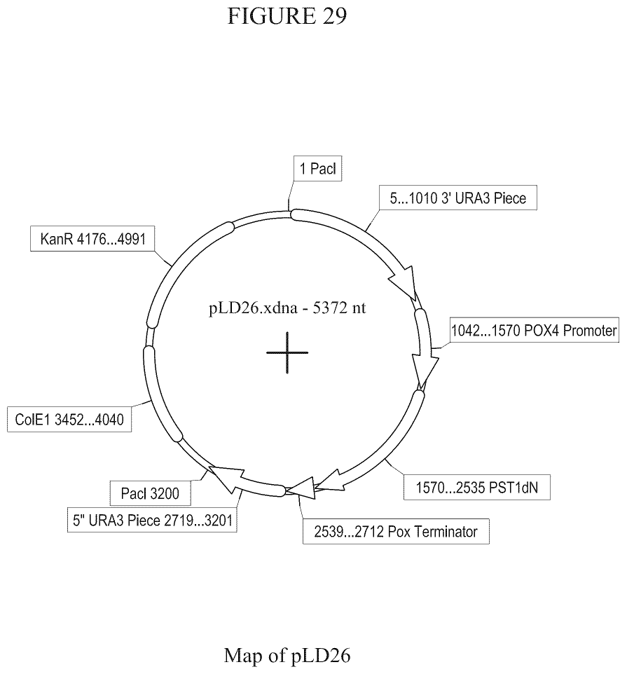

FIG. 30 depicts the use of a fatty acid ester where the fatty acid ester is transported to the cytoplasm. In the cytoplasm, a native or introduce fatty acid esterase cleaves the fatty acid ester to release the fatty acid. This fatty acid is then the substrate for an acyl-CoA synthase forming the corresponding fatty acid CoA. This fatty acid CoA is then the substrate of a polyketide synthase to crease a polyketide.

FIG. 31 depicts the use of a fatty acid ester where the fatty acid ester is cleaved outside of the cell by a native or introduced fatty acid esterase to release the fatty acid. This fatty acid is then transported into the cytoplasm where it is the substrate for an acyl-CoA synthase forming the corresponding fatty acid CoA. This fatty acid CoA is then the substrate of a polyketide synthase to crease a polyketide.

### DETAILED DESCRIPTION

Cannabis is the dried preparation of the Cannabis sativa plant and has been widely used to treat disease or alleviate disease symptoms. The flowers of the plant are used to produce cannabis, but other parts of the plant can be used as well. According to some accounts, cannabis is composed of at least 483 known chemical compounds, which include cannabinoids, terpenoids, flavonoids, nitrogenous compounds, amino acids, proteins, glycoproteins, enzymes, sugars and related compounds, hydrocarbons, alcohols, aldehydes, ketones, acids, fatty acids, esters, lactones, steroids, terpenes, non-cannabinoid phenols, vitamins, and pigments.

The cannabinoids are believed to mediate the medical and recreational properties of the plant. Cannabinoids act by binding to cannabinoid receptors found in the brain to mediate many of the effects of cannabis. The efficacy of cannabinoids for treating specific ailments is the subject of ongoing research with either a purified cannabinoid, a synthetic cannabinoid or cannabis.

For medical applications, the use of a purified cannabinoid is preferred to a mixture of molecules extracted from cannabis. One option for the production of cannabinoids is synthetic biology: the construction of specific strains of bacteria, yeast or filamentous fungi that will produce cannabinoids in a fermentation process. Producing cannabinoids with a genetically modified organism in fermentation has multiple advantages.

A fermentation-based process is more controlled and economical than the current process of isolating cannabinoids from Cannabis sativa plants, which requires expensive indoor facilities and cloning of plant strains under sterile conditions to ensure consistent distribution of cannabinoids in the final plant material.

Usually, cannabinoids are extracted from the cannabis plant as part of a crude mixture, combined with other chemical compounds found in the cannabis plant. Most extractions of cannabis plant matter aim to extract cannabinoids, particularly tetrahydrocannabinol (THC). THC is useful for relieving pain, treating glaucoma, and relieving nausea. THC is also gaining immense popularity as a recreational drug substance. Other cannabinoids of interest include, Cannabigerol (CBG), Cannabigerolic Acid (CBGA), Cannabidiol (CBD), Cannabinol (CBN), Cannabichromene (CBC), Tetrahydrocannabivarin (THCV), Cannabigerovarin (CBGV), and Cannabigerovarinic Acid (CBGVA).

A variety of growing and cultivating techniques have been developed for increasing the production of secondary compounds within plants of genus cannabis. These techniques include outdoor cultivation, indoor cultivation, hydroponics, fertilization, atmospheric manipulation, cloning, crossbreeding, Screen of Grow (SCROG), Sea of Green (SOG), pinching, training, topping, etc.

While breeding and farming techniques yield plants with high concentrations of cannabinoids, these techniques fail to provide the level of control and production needed. In addition, the production time is measured in multiple weeks if not months.

Production of a single cannabinoid by fermentation with a microorganism, will provide the cannabinoid of interest in less complex chemical matrix facilitating the isolation of purified cannabinoid. This will result in less equipment needed and lower cost of purification. In addition, a fermentation-based process timeline will be measured in days and not weeks, allowing production to quickly adapt to changing market needs. Finally, a fermentation-based process footprint will allow production of cannabinoids in a smaller facility than those required for plant-based process where big greenhouses are required.

### Microorganisms

A microorganism selected often is suitable for genetic manipulation and often can be cultured at cell densities useful for industrial production of a target fatty dicarboxylic acid product. A microorganism selected often can be maintained in a fermentation device.

The term “engineered microorganism” as used herein refers to a modified microorganism that includes one or more activities distinct from an activity present in a microorganism utilized as a starting point (hereafter a “host microorganism”). An engineered microorganism includes a heterologous polynucleotide in some embodiments, and in certain embodiments, an engineered organism has been subjected to selective conditions that alter an activity, or introduce an activity, relative to the host microorganism. Thus, an engineered microorganism has been altered directly or indirectly by a human being. A host microorganism sometimes is a native microorganism, and at times is a microorganism that has been engineered to a certain point.

In some embodiments an engineered microorganism is a single cell organism, often capable of dividing and proliferating. A microorganism can include one or more of the following features: aerobe, anaerobe, filamentous, non-filamentous, monoploid, dipoid, auxotrophic and/or non-auxotrophic. In certain embodiments, an engineered microorganism is a prokaryotic microorganism (e.g., bacterium), and in certain embodiments, an engineered microorganism is a non-prokaryotic microorganism. In some embodiments, an engineered microorganism is a eukaryotic microorganism (e.g., yeast, fungi, amoeba). In some embodiments, an engineered microorganism is a fungus. In some embodiments, an engineered organism is a yeast.

Any suitable yeast may be selected as a host microorganism, engineered microorganism, genetically modified organism, or source for a heterologous or modified polynucleotide. Yeast include, but are not limited to, Yarrowia yeast (e.g., Y. lipolytica (formerly classified as Candida lipolytica)), Candida yeast (e.g., C. revkaufi, C. viswanathii, C. pulcherrima, C. tropicalis, C. utilis), Rhodotorula yeast (e.g., R. glutinus, R. graminis), Rhodosporidium yeast (e.g., R. toruloides), Saccharomyces yeast (e.g., S. cerevisiae, S. bayanus, S. pastorianus, S. carlsbergensis), Cryptococcus yeast, Trichosporon yeast (e.g., T. pullans, T. cutaneum), Pichia yeast (e.g., P. pastoris) and Lipomyces yeast (e.g., L. starkeyii, L. lipoferus). In some embodiments, a suitable yeast is of the genus Arachniotus, Aspergillus, Aureobasidium, Auxarthron, Blastomyces, Candida, Chrysosporuim, Chrysosporuim Debaryomyces, Coccidiodes, Cryptococcus, Gymnoascus, Hansenula, Histoplasma, Issatchenkia, Kluyveromyces, Lipomyces, Lssatchenkia, Microsporum, Myxotrichum, Myxozyma, Oidiodendron, Pachysolen, Penicillium, Pichia, Rhodosporidium, Rhodotorula, Rhodotorula, Saccharomyces, Schizosaccharomyces, Scopulariopsis, Sepedonium, Trichosporon, or Yarrowia. In some embodiments, a suitable yeast is of the species Arachniotus flavoluteus, Aspergillus flavus, Aspergillus fumigatus, Aspergillus niger, Aureobasidium pullulans, Auxarthron thaxteri, Blastomyces dermatitidis, Candida albicans, Candida dubliniensis, Candida famata, Candida glabrata, Candida guilliermondii, Candida kefyr, Candida krusei, Candida lambica, Candida lipolytica, Candida lustitaniae, Candida parapsilosis, Candida pulcherrima, Candida revkaufi, Candida rugosa, Candida tropicalis, Candida utilis, Candida viswanathii, Candida xestobii, Chrysosporuim keratinophilum, Coccidiodes immitis, Cryptococcus albidus var. diffluens, Cryptococcus laurentii, Cryptococcus neofomans, Debaryomyces hansenii, Gymnoascus dugwayensis, Hansenula anomala, Histoplasma capsulatum, Issatchenkia occidentalis, lsstachenkia orientalis, Kluyveromyces lactis, Kluyveromyces marxianus, Kluyveromyces thermotolerans, Kluyveromyces waltii, Lipomyces lipoferus, Lipomyces starkeyii, Microsporum gypseum, Myxotrichum deflexum, Oidiodendron echinulatum, Pachysolen tannophilis, Penicillium notatum, Pichia anomala, Pichia pastoris, Pichia stipitis, Rhodosporidium toruloides, Rhodotorula glutinus, Rhodotorula graminis, Saccharomyces cerevisiae, Saccharomyces kluyveri, Schizosaccharomyces pombe, Scopulariopsis acremonium, Sepedonium chrysospermum, Trichosporon cutaneum, Trichosporon pullans, Yarrowia lipolytica, or Yarrowia lipolytica (formerly classified as Candida lipolytica). In some embodiments, a yeast is a Y. lipolytica strain that includes, but is not limited to, ATCC20362, ATCC8862, ATCC18944, ATCC20228, ATCC76982 and LGAM S(7)1 strains (Papanikolaou S., and Aggelis G., Bioresour. Technol. 82(1):43-9 (2002)). In certain embodiments, a yeast is a Candida species (i.e., Candida spp.) yeast. Any suitable Candida species can be used and/or genetically modified for production of a fatty dicarboxylic acid (e.g., octanedioic acid, decanedioic acid, dodecanedioic acid, tetradecanedioic acid, hexadecanedioic acid, octadecanedioic acid, eicosanedioic acid). In some embodiments, suitable Candida species include, but are not limited to Candida albicans, Candida dubliniensis, Candida famata, Candida glabrata, Candida guilliermondii, Candida kefyr, Candida krusei, Candida lambica, Candida lipolytica, Candida lustitaniae, Candida parapsilosis, Candida pulcherrima, Candida revkaufi, Candida rugosa, Candida tropicalis, Candida utilis, Candida viswanathii, Candida xestobii and any other Candida spp. yeast described herein. Non-limiting examples of Candida spp. strains include, but are not limited to, sAA001 (ATCC20336), sAA002 (ATCC20913), sAA003 (ATCC20962), sAA496 (US2012/0077252), sAA106 (US2012/0077252), SU-2 (ura3-/ura3-), H5343 (beta oxidation blocked; U.S. Pat. No. 5,648,247) strains. Any suitable strains from Candida spp. yeast may be utilized as parental strains for genetic modification.

Yeast genera, species and strains are often so closely related in genetic content that they can be difficult to distinguish, classify and/or name. In some cases, strains of C. lipolytica and Y. lipolytica can be difficult to distinguish, classify and/or name and can be, in some cases, considered the same organism. In some cases, various strains of C. tropicalis and C. viswanathii can be difficult to distinguish, classify and/or name (for example see Arie et. al., J. Gen. Appl. Microbiol., 46, 257-262 (2000). Some C. tropicalis and C. viswanathii strains obtained from ATCC as well as from other commercial or academic sources can be considered equivalent and equally suitable for the embodiments described herein. In some embodiments, some parental stains of C. tropicalis and C. viswanathii are considered to differ in name only.

Any suitable fungus may be selected as a host microorganism, engineered microorganism or source for a heterologous polynucleotide. Non-limiting examples of fungi include, but are not limited to, Aspergillus fungi (e.g., A. parasiticus, A. nidulans), Thraustochytrium fungi, Schizochytrium fungi and Rhizopus fungi (e.g., R. arrhizus, R. oryzae, R. nigricans). In some embodiments, a fungus is an A. parasiticus strain that includes, but is not limited to, strain ATCC24690, and in certain embodiments, a fungus is an A. nidulans strain that includes, but is not limited to, strain ATCC38163.

Any suitable prokaryote may be selected as a host microorganism, engineered microorganism or source for a heterologous polynucleotide. A Gram negative or Gram positive bacteria may be selected. Examples of bacteria include, but are not limited to, Bacillus bacteria (e.g., B. subtilis, B. megaterium), Acinetobacter bacteria, Norcardia bacteria, Xanthobacter bacteria, Escherichia bacteria (e.g., E. coli (e.g., strains DH10B, Stb12, DH5-alpha, DB3, DB3.1), DB4, DB5, JDP682 and ccdA-over (e.g., U.S. application Ser. No. 09/518,188)), Streptomyces bacteria, Erwinia bacteria, Klebsiella bacteria, Serratia bacteria (e.g., S. marcessans), Pseudomonas bacteria (e.g., P. aeruginosa), Salmonella bacteria (e.g., S. typhimurium, S. typhi), Megasphaera bacteria (e.g., Megasphaera elsdenii). Bacteria also include, but are not limited to, photosynthetic bacteria (e.g., green non-sulfur bacteria, Chloroflexus bacteria (e.g., C. aurantiacus), Chloronema bacteria (e.g., C. gigateum)), green sulfur bacteria (e.g., Chlorobium bacteria (e.g., C. limicola)), Pelodictyon bacteria (e.g., P. luteolum), purple sulfur bacteria (e.g., Chromatium bacteria (e.g., C. okenii)), and purple non-sulfur bacteria (e.g., Rhodospirillum bacteria (e.g., R. rubrum), Rhodobacter bacteria (e.g., R. sphaeroides, R. capsulatus), and Rhodomicrobium bacteria (e.g., R. vanellii)).

Cells from non-microbial organisms can be utilized as a host microorganism, engineered microorganism or source for a heterologous polynucleotide. Examples of such cells, include, but are not limited to, insect cells (e.g., Drosophila (e.g., D. melanogaster), Spodoptera (e.g., S. frugiperda Sf9 or Sf21 cells) and Trichoplusia (e.g., High-Five cells); nematode cells (e.g., C. elegans cells); avian cells; amphibian cells (e.g., Xenopus laevis cells); reptilian cells; mammalian cells (e.g., NIH3T3, 293, CHO, COS, VERO, C127, BHK, Per-C6, Bowes melanoma and HeLa cells); and plant cells (e.g., Arabidopsis thaliana, Nicotania tabacum, Cuphea acinifolia, Cuphea aequipetala, Cuphea angustifolia, Cuphea appendiculata, Cuphea avigera, Cuphea avigera var. pulcherrima, Cuphea axilliflora, Cuphea bahiensis, Cuphea baillonis, Cuphea brachypoda, Cuphea bustamanta, Cuphea calcarata, Cuphea calophylla, Cuphea calophylla subsp. mesostemon, Cuphea carthagenensis, Cuphea circaeoides, Cuphea confertiflora, Cuphea cordata, Cuphea crassiflora, Cuphea cyanea, Cuphea decandra, Cuphea denticulata, Cuphea disperma, Cuphea epilobiifolia, Cuphea ericoides, Cuphea flava, Cuphea flavisetula, Cuphea fuchsiifolia, Cuphea gaumeri, Cuphea glutinosa, Cuphea heterophylla, Cuphea hookeriana, Cuphea hyssopifolia (Mexican-heather), Cuphea hyssopoides, Cuphea ignea, Cuphea ingrata, Cuphea jorullensis, Cuphea lanceolata, Cuphea linarioides, Cuphea Ilavea, Cuphea lophostoma, Cuphea lutea, Cuphea lutescens, Cuphea melanium, Cuphea melvilla, Cuphea micrantha, Cuphea micropetala, Cuphea mimuloides, Cuphea nitidula, Cuphea palustris, Cuphea parsonsia, Cuphea pascuorum, Cuphea paucipetala, Cuphea procumbens, Cuphea pseudosilene, Cuphea pseudovaccinium, Cuphea pulchra, Cuphea racemosa, Cuphea repens, Cuphea salicifolia, Cuphea salvadorensis, Cuphea schumannii, Cuphea sessiliflora, Cuphea sessilifolia, Cuphea setosa, Cuphea spectabilis, Cuphea spermacoce, Cuphea splendida, Cuphea splendida var. viridiflava, Cuphea strigulosa, Cuphea subuligera, Cuphea teleandra, Cuphea thymoides, Cuphea tolucana, Cuphea urens, Cuphea utriculosa, Cuphea viscosissima, Cuphea watsoniana, Cuphea wrightii, Cuphea lanceolata)).

Microorganisms or cells used as host organisms or source for a heterologous polynucleotide are commercially available. Microorganisms and cells described herein, and other suitable microorganisms and cells are available, for example, from Invitrogen Corporation (Carlsbad, Calif.), American Type Culture Collection (Manassas, Va.), and Agricultural Research Culture Collection (NRRL; Peoria, Ill.).

Host microorganisms and engineered microorganisms may be provided in any suitable form. For example, such microorganisms may be provided in liquid culture or solid culture (e.g., agar-based medium), which may be a primary culture or may have been passaged (e.g., diluted and cultured) one or more times. Microorganisms also may be provided in frozen form or dry form (e.g., lyophilized). Microorganisms may be provided at any suitable concentration.

### Important Pathways for Cannabinoid Production—Beta Oxidation

Cellular fatty acid degradation occurs via the β-oxidation pathway in all organisms. See, e.g., European Published Application No. EP2502932A1. So far it has been established that there are two different β-oxidation systems in eukaryotes: the β-oxidation located in mitochondria for mammals and some filamentous fungi and the β-oxidation system located in peroxisomes for plants, fungi, and animals.

Fatty acid beta-oxidation begins with the addition of coenzyme A to a fatty acid and occurs by successive cycles of reactions during each of which the fatty acid is shortened by a two-carbon fragment removed as acetyl coenzyme A, generating trans-2,3 hydroxyl, and 3-keto intermediates, until only two or three carbons remain (as acetyl-CoA or propionyl-CoA respectively). The proteins involved in the mitochondrial β-oxidation and in the peroxisomal 3-oxidation are however different. Multifunctional proteins (MFPs) or multifunctional enzymes (MFEs) are involved in the peroxisomal β-oxidation pathway, whereas β-oxidation consists of monofunctional enzymes.

The peroxisomal β-oxidation process begins with oxidation of the acyl-CoA substrate into trans-2-enoyl-CoA by Acyl-CoA oxidase, namely Fox1p/Pox1p. It has been demonstrated that Pox1Δ yeasts are unable to grow on fatty acids as sole carbon atoms. Then the peroxisomal β-oxidation proceeds from trans-2-enoyl-CoA to 3-ketoacyl-CoA via the (3R)-hydroxyacyl-CoA ester intermediates. In the yeast oxidation system, the second and third reactions of the β-oxidation cycle are catalyzed by the same enzymes called Mfe2p, Fox2p or again Pox2p, which contains both the 3-hydroxyacyl-CoA and 2-enoyl-CoA hydratase activities. The 2-enoyl-CoA hydratase converts the trans-2-enoyl CoA esters into (3R)-hydroxyacyl-CoA esters, whereas the hydratase 2 produces the 3-ketoacyl-CoA. This enzyme was first isolated from Candida tropicalis and comprise a duplicated domain organization in its N-terminal region, which contains two deshydrogenase active domains A and B. Domain A was demonstrated to have highest activity with long and medium chain substrates, whereas domain B has the highest activity with short-chain substrates. The C-terminal region of the Fox2p enzyme contains the 2-enoyl-CoA hydratase 2 activity. Hiltunen et al. (JBC, Vol. 267, No. 10, Apr. 5, 1992, pp 6646-6653) showed that fatty acid catabolism in yeast was mainly based on the activity of Fox2p and that disruption of FOX2 resulted in the inability of yeast cells to grow on fatty acids as their sole carbon source. At the next reaction of the β-oxidation cycle the ketoacyl-CoA intermediate undergoes thiolytic cleavage by a 3-ketoacyl-CoA thiolase, namely Pot1p/Fox3p. The Pot1p/Fox3p is a dimeric protein with a subunit size of 45 kDa. A single subunit comprises three domains: two core domains, and a loop domain of 120 residues. The active site of yeast thiolase is shaped by residues from the two core domains and surrounded by the loop domain. The products of this last step are acetyl-CoA and a C2-shortened acyl-CoA, which acts as substrate for Pox1 p/Fox1 p for an additional cycle. The acetyl-CoA which is produced by peroxisomal beta oxidation is then used in the glyoxylic cycle, thereby allowing the transformation of acetyl-CoA into oxaloacetate. These reactions are catalyzed by two enzymes: isocitrate lyase (Icl1p) and malate synthase (Mls1p) which permits the use of two carbon atoms such as acetate, in the neoglucogenese.

### Cannabinoid Production

Acyl-CoA oxidase (EC 1.3.3.6) is the first reported enzyme of the fatty acid 3-oxidation pathway. See, e.g., U.S. Pat. No. 6,518,488. This enzyme catalyzes the desaturation of acyl-CoAs longer than eight carbons to 2-trans-enoyl-CoAs, by donating electrons directly to molecular oxygen and releasing H₂O₂(Lazarow et al., 1976). There are multiple isozymes of acyl-CoA oxidase and these isozymes show specificity towards short, medium, and long chain fatty acyl-CoAs (Hooks et al., Biochem J., 320:607-614 (1996); Hooks et al., Plant J., 20:1-13 (1999)). For example, Arabidopsis thaliana acyl-CoA oxidase isoform 1 (ACX1) has optimal activity on an acyl-CoA substrate that is fourteen carbons long and minimal activity on substrates shorter than six carbons. However, ACX2 has optimal activity on an acyl-CoA substrate that is eighteen carbons long and minimal activity on substrates shorter than ten carbons. In Y. lipolytica, there are five acyl-CoA oxidase isoforms that have different activities on acyl-CoA substrates of different lengths. For example, the protein encoded by POX3 has maximal activity on C6 and C8 acyl-CoA substrates.

Cannabinoids have their biosynthetic origins in both polyketide and terpenoid metabolism and are termed terpenophenolics or prenylated polyketides (See, e.g., US Patent Publication No. US20190169661; Page J., Nagel J. (2006) Biosynthesis of terpenophenolics in hop and cannabis. In J T Romeo, ed, Integrative Plant Biochemistry, Vol. 40. Elsevier, Oxford, pp 179-210.).

Polyketides represent a large family of diverse compounds ultimately synthesized from 2-carbon units through a series of Claisen-type condensations and subsequent modifications. See, e.g., US Patent Publication No. US20050032176. Members of this group include antibiotics such as tetracyclines, anticancer agents such as daunomycin, and immunosuppressants such as FK506 and rapamycin. Polyketides occur in many types of organisms including fungi and mycelial bacteria, in particular, the actinomycetes.

The structural diversity of polyketides is achieved through the series of reactions catalyzed by polyketide synthases (PKS), with features that contribute to diversity including the selection of various starter and extender units, final chain length, cyclization, degree of reduction, and the like. See, e.g., US20120122180. Downstream reactions such as glycosylation, hydroxylation, halogenation, prenylation, acylation, and alkylation can add additional diversity to the resulting products. This group of enzymatically active proteins is considered in a different category from the fatty acid synthases which also catalyze condensation of 2-carbon units to result in, for example, fatty acids and prostaglandins. Two major types of PKS are known which are vastly different in their construction and mode of synthesis. These are commonly referred to as Type I or “modular” and Type II, “aromatic.”

There is a third class of PKS enzymes, the Type III PKS synthases, which consist of a small homodimer containing one active site where both chain extension and cyclization take place (See. e.g., US20190078098; Austin, M. B. and J. P. Noel. Natural Product Reports, 2002. 20(1): p. 79-110; Lim, Y., et al. Molecules, 2016.21(6): p. 806; Yu, D., et al. IUBMB Life, 2012. 64(4): p. 285-295). Type III PKSs are able to produce a wide diversity of polyketide products by using a variety of larger, CoA-containing precursors as a starting unit. These starters range from small aliphatic molecules, such as acetyl-CoA, to larger ring-containing compounds derived from the phenylpropanoid pathway, such as 4-coumaroyl-CoA. Often, these CoA molecules are formed through the function of acid CoA ligases that convert carboxylic acids into corresponding CoA molecules.

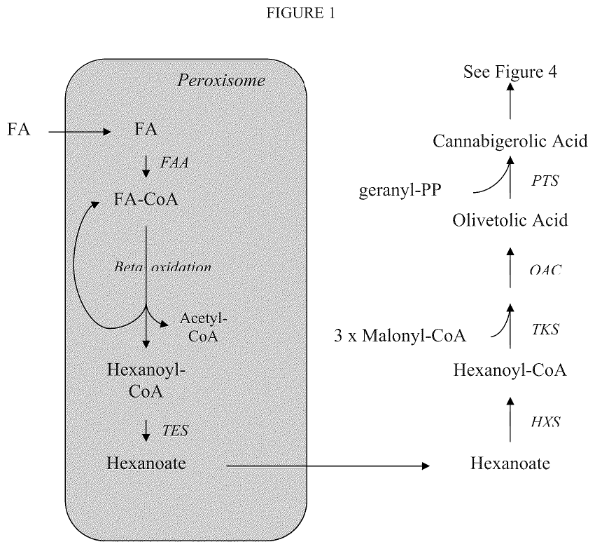

Cannabinoid biosynthesis occurs primarily in glandular trichomes that cover female flowers at a high density. See, e.g., US20190169661. Cannabinoids are formed by a three-step biosynthetic process: polyketide formation, aromatic prenylation and cyclization (see FIG. 1).

The first enzymatic step in cannabinoid biosynthesis is the formation of olivetolic acid by a putative polyketide synthase enzyme that catalyzes the condensation of hexanoyl coenzyme A (CoA) and malonyl CoA. A Type III polyketide synthase, termed “olivetol synthase” and referred to herein as polyketide synthase/olivetol synthase (CsPKS/olivetol synthase), from Cannabis sativa has recently been shown to form olivetol and several pyrone products but not olivetolic acid (Taura F, Tanaka S, Taguchi C, Fukamizu T, Tanaka H, Shoyama Y, Morimoto, S. (2009) Characterization of olivetol synthase, Type III a polyketide synthase putatively involved in cannabinoid biosynthetic pathway. FEBS Lett. 583: 2061-2066.). The nucleotide sequence of the gene encoding CsPKS/olivetol synthase is found in GenBank under accession number AB164375 with the polypeptide as accession BAG14339. The aforementioned products include the pyrones hexanoytriacetic lactone (HTAL) and pentyldiacetic lactone (PDAL). The reason for the inability of this enzyme to form olivetolic acid, which is clearly a pathway intermediate based on the carboxylate structure of the cannabinoids, is not known. The lack of olivetolic acid formation by this polyketide synthase from cannabis was confirmed by the inventors, as further described herein and also by Marks et al. (Marks M D, Tian L, Wenger J P, Omburo S N, Soto-Fuentes W, He J, Gang D R, Weiblen G D, Dixon R A. (2009) Identification of candidate genes affecting Delta9-tetrahydrocannabinol biosynthesis in Cannabis sativa. J Exp Bot. 60, 3715-3726.).

The second enzymatic step is the prenylation of olivetolic acid to form cannabigerolic acid (CBGA) by the enzyme geranylpyrophosphate:olivetolate geranyltransferase. This enzyme is an aromatic prenyltransferase and is the subject of commonly owned U.S. Provisional patent applications U.S. Ser. No. 61/272,057 filed Aug. 12, 2009 and U.S. Ser. No. 61/272,117 filed Aug. 18, 2009. CBGA is a central branch-point intermediate for the biosynthesis of the different classes of cannabinoids. Cyclization of CBGA yields Δ9-tetrahydrocannabinolic acid (THCA) or its isomers cannabidiolic acid (CBDA) or cannabichromenic acid (CBCA) (see FIG. 1). The Shoyama group has previously published the identification and purification of the three enzymes responsible for these cyclizations (Morimoto S, Komatsu K, Taura F, Shoyama, Y. (1998) Purification and characterization of cannabichromenic acid synthase from Cannabis sativa. Phytochemistry. 49: 1525-1529; Taura F, Morimoto S, Shoyama Y. (1996) Purification and characterization of cannabidiolic-acid synthase from Cannabis sativa L. Biochemical analysis of a novel enzyme that catalyzes the oxidocyclization of cannabigerolic acid to cannabidiolic acid. J Biol Chem. 271: 17411-17416; and Taura F, Morimoto S, Shoyama Y, Mechoulam R. (1995) First direct evidence for the mechanism of 1-tetrahydrocannabinolic acid biosynthesis. J Am Chem Soc. 117: 9766-9767). Cloning of THCA and CBDA synthases has also been previously published (Sirikantaramas S, Taura F, Tanaka Y, Ishikawa Y, Morimoto S, Shoyama Y. (2005) Tetrahydrocannabinolic acid synthase, the enzyme controlling marijuana psychoactivity, is secreted into the storage cavity of the glandular trichomes. Plant Cell Physiol. 46: 1578-1582.; Taura F, Sirikantaramas S, Shoyama Y, Yoshikai K, Shoyama Y, Morimoto S. (2007) Cannabidiolic-acid synthase, the chemotype-determining enzyme in the fiber-type Cannabis sativa. FEBS Lett. 581: 2929-2934. The genes for THCA synthase and CBDA synthase have been reported in Japan (Japanese Patent Publication 2000-078979; Japanese Patent Publication 2001-029082).

### Beta-Oxidation Activities

The term “beta oxidation pathway” as used herein, refers to a series of enzymatic activities utilized to metabolize fatty alcohols, fatty acids, or dicarboxylic acids. The activities utilized to metabolize fatty alcohols, fatty acids, or dicarboxylic acids include, but are not limited to, acyl-CoA ligase activity, acyl-CoA oxidase activity, acyl-CoA hydrolase activity, acyl-CoA thioesterase activity, enoyl-CoA hydratase activity, 3-hydroxyacyl-CoA dehydrogenase activity and acetyl-CoA C-acyltransferase activity. The term “beta oxidation activity” refers to any of the activities in the beta oxidation pathway utilized to metabolize fatty alcohols, fatty acids, or dicarboxylic acids.

### Beta-Oxidation—Acyl-CoA Ligase

An acyl-CoA ligase enzyme sometimes is encoded by the host organism and can be added to generate an engineered organism. In some embodiments, host acyl-CoA ligase activity can be increased by increasing the number of copies of an acyl-CoA ligase gene, by increasing the activity of a promoter that regulates transcription of an acyl-CoA ligase gene, or by increasing the number copies of the gene and by increasing the activity of a promoter that regulates transcription of the gene, thereby increasing production of target due to increased carbon flux through the pathway. In certain embodiments, the acyl-CoA ligase gene can be isolated from any suitable organism. Non-limiting examples of organisms that include, or can be used as donors for, acyl-CoA ligase enzymes include Arxula, Candida, Saccharomyces, or Yarrowia.

### Beta-Oxidation-Enoyl-CoA Hydratase

An enoyl-CoA hydratase enzyme catalyzes the addition of a hydroxyl group and a proton to the unsaturated β-carbon on a fatty-acyl CoA and sometimes is encoded by the host organism and sometimes can be added to generate an engineered organism. In certain embodiments, the enoyl-CoA hydratase activity is unchanged in a host or engineered organism. In some embodiments, the host enoyl-CoA hydratase activity can be increased by increasing the number of copies of an enoyl-CoA hydratase gene, by increasing the activity of a promoter that regulates transcription of an enoyl-CoA hydratase gene, or by increasing the number copies of the gene and by increasing the activity of a promoter that regulates transcription of the gene, thereby increasing the production of target product (due to increased carbon flux through the pathway. In certain embodiments, the enoyl-CoA hydratase gene can be isolated from any suitable organism. Non-limiting examples of organisms that include, or can be used as donors for, enoyl-CoA hydratase enzymes include Arxula, Candida, Saccharomyces, or Yarrowia.

### Beta-Oxidation-3-Hydroxyacyl-CoA Dehydrogenase

3-hydroxyacyl-CoA dehydrogenase enzyme catalyzes the formation of a 3-ketoacyl-CoA by removal of a hydrogen from the newly formed hydroxyl group created by the activity of enoyl-CoA hydratase. In some embodiments, the activity is encoded by the host organism and sometimes can be added or increased to generate an engineered organism. In certain embodiments, the 3-hydroxyacyl-CoA activity is unchanged in a host or engineered organism. In some embodiments, the host 3-hydroxyacyl-CoA dehydrogenase activity can be increased by increasing the number of copies of a 3-hydroxyacyl-CoA dehydrogenase gene, by increasing the activity of a promoter that regulates transcription of a 3-hydroxyacyl-CoA dehydrogenase gene, or by increasing the number copies of the gene and by increasing the activity of a promoter that regulates transcription of the gene, thereby increasing production of target product (e.g., sebacic or dodecanedioic acid) due to increased carbon flux through the pathway. In certain embodiments, the 3-hydroxyacyl-CoA dehydrogenase gene can be isolated from any suitable organism. Non-limiting examples of organisms that include, or can be used as donors for, 3-hydroxyacyl-CoA dehydrogenase enzymes include Arxula, Candida, Saccharomyces, or Yarrowia.

### Beta-Oxidation-Acetyl-CoA C-Acyltransferase

An Acetyl-CoA C-acyltransferase (e.g., beta-ketothiolase) enzyme catalyzes the formation of a fatty acyl-CoA shortened by 2 carbons by cleavage of the 3-ketoacyl-CoA by the thiol group of another molecule of CoA. The thiol is inserted between C-2 and C-3, which yields an acetyl CoA molecule and an acyl CoA molecule that is two carbons shorter. An Acetyl-CoA C-acyltransferase sometimes is encoded by the host organism and sometimes can be added to generate an engineered organism. In certain embodiments, the acetyl-CoA C-acyltransferase activity is unchanged in a host or engineered organism. In some embodiments, the host acetyl-CoA C-acyltransferase activity can be increased by increasing the number of copies of an acetyl-CoA C-acyltransferase gene, or by increasing the activity of a promoter that regulates transcription of an acetyl-CoA C-acyltransferase gene, thereby increasing the production of target product due to increased carbon flux through the pathway. In certain embodiments, the acetyl-CoA C-acyltransferase gene can be isolated from any suitable organism. Non-limiting examples of organisms that include, or can be used as donors for, acetyl-CoA C-acyltransferase enzymes include Arxula, Candida, Saccharomyces, or Yarrowia.

### Altered Activities and Engineering Pathways

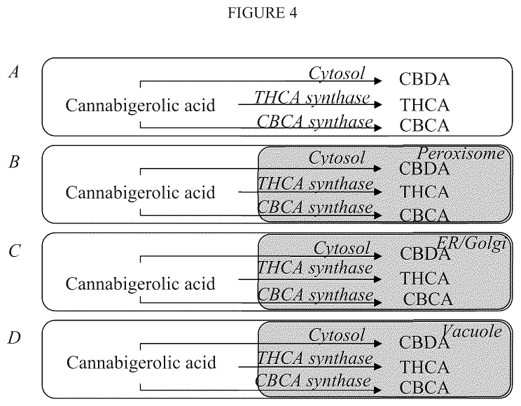

In one embodiment, which is represented by FIG. 1, the microorganism is engineered to consume fatty acids through peroxisomal beta-oxidation by insertion of a gene encoding an acyl-CoA oxidase. The acyl-CoA oxidase is targeted to the peroxisome by the addition of a peroxisomal targeting sequence (PTS) to the carboxyl-terminus to the protein. A PTS sequence can be GRRAKL or a smaller subset of those amino acids based on the consensus sequence [S/A/H/C/E/P/Q/V]-[K/R/H/Q]-[L/F]. Alternatively, a microorganism that naturally consumes fatty acids can be used. The microorganism will be constructed to express a hexanoate-acyl activating enzyme (HXS), an olivetol synthase (TKS), an olivetol cyclase (OAC), a cannabigerolic acid synthase (PTS), and either a cannabidiolic synthase (CBDAS) to produce cannabidiolic acid, a tetrahydrocannabinolic acid synthase (THCAS) to produce tetrahydrocannabinolic acid, or a cannabichromenic acid synthase (CBCAS) to produce cannabichromenic acid. The CBDAS, THCAS or CBCAS may be localized to either the cytosol, the peroxisome, a secretory traffic compartment such as the ER or Golgi or the vacuole through the use of signal sequences as described in FIG. 4. Other genetic manipulations may be performed that are known to increase the carbon flux into the isoprenoid pathway.

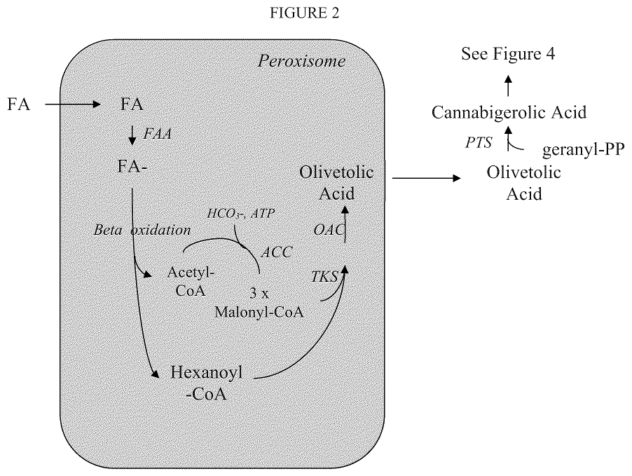

In one embodiment, which is represented by FIG. 2, the microorganism is engineered to consume fatty acids through peroxisomal beta-oxidation by insertion of a gene encoding an acyl-CoA oxidase. The acyl-CoA oxidase is targeted to the peroxisome by the addition of a peroxisomal targeting sequence (PTS) to the carboxyl-terminus of the protein. A PTS sequence can be GRRAKL or a smaller subset of those amino acids based on the consensus sequence [S/A/H/C/E/P/Q/V]-[K/R/H/Q]-[L/F]. Alternatively, a microorganism that naturally consumes fatty acids can be used. The microorganism will be constructed to express an olivetol synthase (TKS), an olivetol cyclase (OAC), a cannabigerolic acid synthase (PTS), and either a cannabidiolic synthase (CBDAS) to produce cannabidiolic acid, a tetrahydrocannabinolic acid synthase (THCAS) to produce tetrahydrocannabinolic acid, or a cannabichromenic acid synthase (CBCAS) to produce cannabichromenic acid. The TKS and OAC enzymes will be targeted to the peroxisome by the addition of a PTS sequence, such as GRRAKL or a smaller subset of those amino acids based on the consensus sequence of [S/A/H/C/E/P/Q/V]-[K/R/H/Q]-[L/F], which will be added to the carboxyl terminus of the proteins. An alternative mechanism of targeting to the peroxisome is the use of a PTS2 sequence near the N-terminus of the protein, which is defined by the consensus sequence -(R/K)(L/V/I/Q)XX(L/V/I/H/Q)(L/S/G/A/K)X(H/Q)(L/A/F)-. An example could be RRMLSSKQL as found in Pcdlp from S. cerevisiae. The CBDAS, THCAS or CBCAS may be localized to either the cytosol, the peroxisome, a secretory traffic compartment such as the ER or Golgi or the vacuole through the use of signal sequences as described in FIG. 4. Other genetic manipulations may be performed that are known to increase the carbon flux into the isoprenoid pathway.

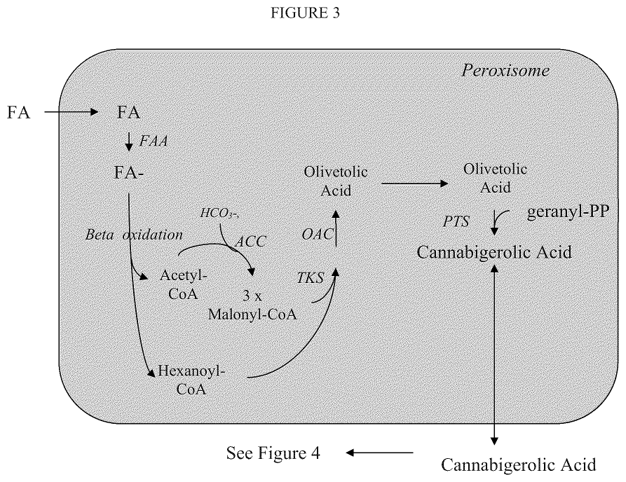

In one embodiment, which is represented by FIG. 3, the microorganism is engineered to consume fatty acids through peroxisomal beta-oxidation by insertion of a gene encoding an acyl-CoA oxidase. The acyl-CoA oxidase is targeted to the peroxisome by the addition of a peroxisomal targeting sequence (PTS) to the carboxyl-terminus of the protein. A PTS sequence can be GRRAKL or a smaller subset of those amino acids based on the consensus sequence of [S/A/H/C/E/P/Q/V]-[K/R/H/Q]-[L/F]. Alternatively, a microorganism that naturally consumes fatty acids can be used. The microorganism will be constructed to express an olivetol synthase (TKS), an olivetol cyclase (OAC), a cannabigerolic acid synthase (PTS), and either a cannabidiolic synthase (CBDAS) to produce cannabidiolic acid, a tetrahydrocannabinolic acid synthase (THCAS) to produce tetrahydrocannabinolic acid, or a cannabichromenic acid synthase (CBCAS) to produce cannabichromenic acid. The TKS, OAC and PTS enzymes will be targeted to the peroxisome by the addition of a PTS sequence, such as GRRAKL or a smaller subset of those amino acids based on the consensus sequence of [S/A/H/C/E/P/Q/V]-[K/R/H/Q]-[L/F], which will be added to the carboxyl terminus of the proteins. An alternative mechanism of targeting to the peroxisome is the use of a PTS2 sequence near the N-terminus of the protein, which is defined by the consensus sequence -(R/K)(L/V/I/Q)XX(L/V/I/H/Q)(L/S/G/A/K)X(H/Q)(L/A/F)-. An example could be RRMLSSKQL as found in Pcd1p from S. cerevisiae. If necessary, an acetyl-CoA carboxylase will be expressed and targeted to the peroxisome through a PTS1 or PTS2. The CBDAS, THCAS or CBCAS may be localized to either the cytosol, the peroxisome, a secretory traffic compartment such as the ER or Golgi or the vacuole through the use of signal sequences as described in FIG. 4. Other genetic manipulations may be performed that are known to increase the carbon flux into the isoprenoid pathway.

In one embodiment, which is represented by “A” of FIG. 4, the THCA synthase, CBDA synthase or CBCA synthase enzyme is expressed in the cytoplasm or cytosol as the native form of the enzyme.

In one embodiment, which is represented by “B” of FIG. 4, the THCA synthase, CBDA synthase or CBCA synthase enzyme is targeted to the vacuole by the addition of a KFERQ motif to the enzyme or the addition of an N-terminal QRPL motif found on carboxypeptidase Y from Saccharomyces cerevisiae. An alternative vacuolar targeting sequence is the hydrophobic 22 amino acid signal sequence, or pre sequence (MFSLKALLPLALLLVSANQVAA) (SEQ ID NO: 1), and 54 amino acid propeptide (KVHKAKIYKHELSDEMKEVTFEQHLAHLGQKYLTQFEKANPEVVFSREHPFFTE) (SEQ ID NO: 2) of pre-pro Proteinase A from Saccharomyces cerevisiae or of the particular yeast that will be engineered for cannabinoid production. Deletions in proteinases, such as PEP4, may be performed to ensure the targeted enzyme is not degraded in the vacuole.

In one embodiment, which is represented by “C” of FIG. 4, the THCA synthase, CBDA synthase or CBCA synthase enzyme is targeted to the endoplasmic reticulum (ER), Golgi or generally to the secretory pathway by the addition of some or all of the pre-pro region of alpha-factor from Saccharomyces cerevisiae or the Ostlp signal sequence from Saccharomyces cerevisiae. The alpha-factor or Ostlp secretion signal from the non-conventional yeast that will be engineered will be identified and considered as well. Other possible secretion signals include the 22 amino acid pre sequence of Proteinase A from Saccharomyces cerevisiae, MVRMVPVLLSLLLLLGPA (SEQ ID NO: 3) from human zinc-binding alpha-2-glycoprotein, MLFSNTLLIAAASALLAEA (SEQ ID NO: 4) from Kluyveromyces marxianus polygalacturonase, and MKFGVLFSVFAAIVSALPA (SEQ ID NO: 5) from Saccharomycopsis fibuligera glucoamylase. An HDEL motif can be added to the C-terminus of a protein to ensure retention in the ER.

In one embodiment, which is represented by “D” FIG. 4, the THCA synthase, CBDA synthase or CBCA synthase enzyme is targeted to the peroxisome by the addition of some or all of the PTS sequence GRRAKL (SEQ ID NO: 6) or a smaller subset of those amino acids based on the consensus sequence of [S/A/H/C/E/P/Q/V]-[K/R/H/Q]-[L/F] (SEQ ID NO: 7) to the C-terminus of each protein. An alternative mechanism of targeting to the peroxisome is the use of a PTS2 sequence near the N-terminus of the protein, which is defined by the consensus sequence -(R/K)(L/V/I/Q)XX(L/V/I/H/Q)(L/S/G/A/K)X(H/Q)(L/A/F)- (SEQ ID NO: 8). An example could be RRMLSSKQL (SEQ ID NO: 9) as found in Pcdlp from S. cerevisiae.

One of the intermediates for the production of some polyketides a fatty is acid. For example, an intermediate for cannabinoids is hexanoic acid. This six-carbon liner carboxylic acid is toxic to yeast at low concentration. This lipophilic weak acid crosses the plasma membrane by passive diffusion and dissociate in the neutral cytosol leading to a decrease in the intracellular pH and accumulating. This cause inhibition of growth and death. For example, at pH 5, the specific growth of Saccharomyces cerevisiae in minimum media drops from 0.4 hr-1 to 0.22 hr-1 at a hexanoic concentration of 2 mm (0.3 g/L). The inhibition and toxicity of hexanoic acid and some other short fatty acids makes it a challenge to run a fermentation with it. The fermentation will need to be run in a fed batch form adding hexanoic acid at a very low rate to avoid accumulating and causing toxicity. The fermentation will be needed to run at a higher pH where the toxicity of fatty acids is less. This will make the fermentation more prone to contamination. Some short fatty acids such as hexanoic acid can be corrosive to metals so special metallurgy needs to be used for the piping of the feedstock.

Herein, also disclosed is the use of fatty acid esters for the production of polyketides by fermentation. Some suitable fatty acid esters include methyl, ethyl, butyl, allyl, isobutyl, hexyl, propyl, and geranyl fatty acid esters. Of special interest are ethyl esters, as the ethyl group can be used to produce acetyl coA as a carbon source, and geraryl caproate as it will provide two intermediates for the cannabinoid pathway. In addition, this process may require the expression of an esterase to slowly cleave the fatty acid ester if there is not an endogenous esterase being produced.

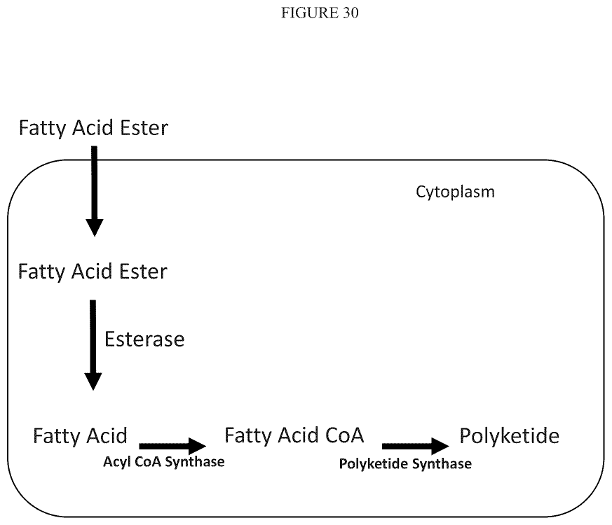

In one embodiment as illustrated in FIG. 30 for the production of the polyketides, a strain from yeast that had been modified to produce polyketides from a fatty and dextrose, is modified to express an esterase or it has an endogenous cytoplasmic fatty acid esterase.

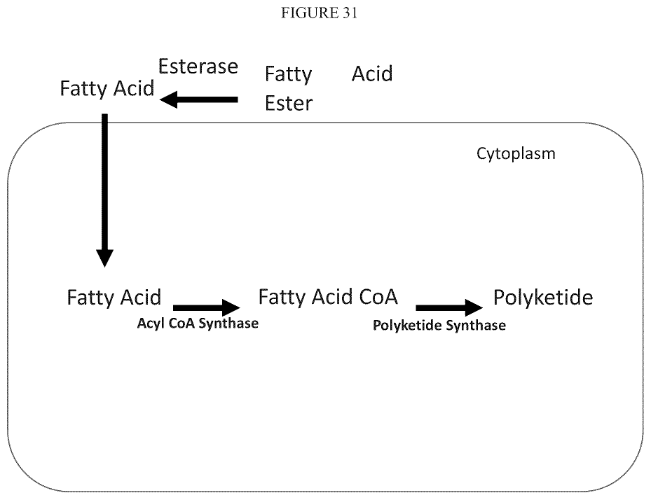

In another embodiment as illustrated in FIG. 31, a strain from a yeast that had been modified to produce cannabinoids from hexanoic acid and dextrose is modified to express a hexanoate esterase or it has an endogenous secreted hexanoate esterase. The hexanoate esterase has been targeted for secretion by a terminal fusion with a secretion motif such as the leader sequence of the yeast mating pheromone alpha-factor or invertase. In addition to the motifs mentioned here, other motifs could be used.

Some potential sources for the hexanoate ester are as follows: Lactobacillus casei EstB, Lactobacillus plantarum Lp_0796, Acinetobacter sp. ADP1 AreA, and Lactococcus lactis EstA.

The methods used to construct these strains are commonly known, have been used extensively to engineer S. cerevisiae and non-conventional yeasts and are described in numerous scientific publications and patents. Promoters will be used that are active during the preferred fermentation conditions. Some examples of promoters that could be used are those of the genes encoding glyceraldehyde 3-phosphate dehydrogenase and the translational elongation factor EF-1 alpha. Genes will be inserted in intergenic regions or non-essential genes.

### Sources for Enzymes

Expressing one of these proteins in an acyl-CoA oxidase null mutant may result in the production of hexanoate CoA.

ACO1P Protein (Source Anthrobacter)
(SEQ ID NO: 10)
MTEVVDRASSPASPGSTTAAADGAKVAVEPRVDVA
ALGEQLLGRWADIRLHARDLAGREVVQKVEGLTHT
EHRSRVFGQLKYLVDNNAVHRAFPSRLGGSDDHGG
NIAGFEELVTADPSLQIKAGVQWGLFGSAVMHLGT
REHHDKWLPGIMSLEIPGCFAMTETGHGSDVASIA
TTATYDEETQEFVIDTPFRAAWKDYIGNAANDGLA
AVVFAQLITRKVNHGVHAFYVDLRDPATGDFLPGI
GGEDDGIKGGLNGIDNGRLHFTNVRIPRTNLLNRY
GDVAVDGTYSSTIESPGRRFFTMLGTLVQGRVSLD
GAAVAASKVALQSAIHYAAERRQFNATSPTEEEVL
LDYQRHQRRLFTRLATTYAASFAHEQLLQKFDDVF
SGARDTDADRQDLETLAAALKPLSTWHALDTLQEC
REACGGAGFLIENRFASLRADLDVYVTFEGDNTVL
LQLVAKRLLADYAKEFRGANFGVLARYVVDQAAGV
ALHRTGLRQVAQFVADSGSVQKSALALRDEEGQRT
LLTDRVQSMVAEVGAALKGAGKLPQHQAAALFNQH
QNELIEAAQAHAELLQWEAFTEALAKVDDAGTKEV
LTRLRDLFGLSLIEKHLSWYLMNGRLSMQRGRTVG
TYINRLLVKIRPHALDLVDAFGYGAEHLRAAIATG
AEATRQDEARTYFRQQRASGSAPADEKTLLAIKAG
KSRGRRAKL
ACO2 Protein
(Source Yarrowia lipolytica)
(SEQ ID NO: 11)
MNPNNTGTIEINGKEYNTFTEPPVAMAQERAKTSF
PVREMTYFLDGGEKNTLKNEQIMEEIERDPLFNND
NYYDLNKEQIRELTMERVAKLSLFVRDQPEDDIKK
RFALIGIADMGTYTRLGVHYGLFFGAVRGTGTAEQ
FGHWISKGAGDLRKFYGCFSMTELGHGSNLAGLET
TAIYDEETDEFIINTPHIAATKWWIGGAAHTATHT
VVFARLIVKGKDYGVKTFVVQLRNINDHSLKVGIS
IGDIGKKMGRDGIDNGWIQFTNVRIPRQNLLMKYT
KVDREGNVTQPPLAQLTYGSLITGRVSMASDSHQV
GKRFITIALRYACIRRQFSTTPGQPETKIIDYPYH
QRRLLPLLAYVYALKMTADEVGALFSRTMLKMDDL
KPDDKAGLNEVVSDVKELFSVSAGLKAFSTWACAD
VIDKTRQACGGHGYSGYNGFGQAYADWVVQCTWEG
DNNILTLSAGRALIQSAVALRKGEPVGNAVSYLKR
YKDLANAKLNGRSLTDPKVLVEAWEVAAGNIINRA
TDQYEKLIGEGLNADQAFEVLSQQRFQAAKVHTRR
HLIAAFFSRIDTEAGEAIKQPLLNLALLFALWSIE
EDSGLFLREGFLEPKDIDTVTELVNKYCTTVREEV
IGYTDAFNLSDYFINAPIGCYDGDAYRHYFQKVNE
QNPARDPRPPYYASTLKPFLFREEEDDDICELDEE
ACO3 Protein
(Source Rattus norvegicus)
(SEQ ID NO: 12)
MNPDLRKERASATFNPELITHILDGSPENTRRRRE
IENLILNDPDFQHEDYNFLTRSQRYEVAVKKSATM
VKKMREYGISDPEEIMWFKKLYLANFVEPVGLNYS
MFIPTLLNQGTTAQQEKWMRPSQELQIIGTYAQTE
MGHGTHLRGLETTATYDPKTQEFILNSPTVTSIKW
WPGGLGKTSNHAIVLAQLITQGECYGLHAFVVPIR
EIGTHKPLPGITVGDIGPKFGYEEMDNGYLKMDNY
RIPRENMLMKYAQVKPDGTYVKPLSNKLTYGTMVF
VRSFLVGNAAQSLSKACTIAIRYSAVRRQSEIKQS
EPEPQILDFQTQQYKLFPLLATAYAFHFVGRYMKE
TYLRINESIGQGDLSELPELHALTAGLKAFTTWTA
NAGIEECRMACGGHGYSHSSGIPNIYVTFTPACTF
EGENTVMMLQTARFLMKIYDQVRSGKLVGGMVSYL
NDLPSQRIQPQQVAVWPTMVDINSLEGLTEAYKLR
AARLVEIAAKNLQTHVSHRKSKEVAWNLTSVDLVR
ASEAHCHYVVVKVFSDKLPKIQDKAVQAVLRNLCL
LYSLYGISQKGGDFLEGSIITGAQLSQVNARILEL
LTLIRPNAVALVDAFDFKDMTLGSVLGRYDGNVYE
NLFEWAKKSPLNKTEVHESYHKHLKPLQSKL
ACO4 Protein
(Source Rattus norvegicus)
(SEQ ID NO: 13)
MNPDLRKERASATFNPELITHILDGSPENTRRRRE
IENLILNDPDFQHEDYNFLTRSQRYEVAVKKSATM
VKKMREYGISDPEEIMWFKNSVHRGHPEPLDLHLG
MFLPTLLHQATAEQQERFFMPAWNLEITGTYAQTE
MGHGTHLRGLETTATYDPKTQEFILNSPTVTSIKW
WPGGLGKTSNHAIVLAQLITQGECYGLHAFVVPIR
EIGTHKPLPGITVGDIGPKFGYEEMDNGYLKMDNY
RIPRENMLMKYAQVKPDGTYVKPLSNKLTYGTMVF
VRSFLVGNAAQSLSKACTIAIRYSAVRRQSEIKQS
EPEPQILDFQTQQYKLFPLLATAYAFHFVGRYMKE
TYLRINESIGQGDLSELPELHALTAGLKAFTTWTA
NAGIEECRMACGGHGYSHSSGIPNIYVTFTPACTF
EGENTVMMLQTARFLMKIYDQVRSGKLVGGMVSYL
NDLPSQRIQPQQVAVWPTMVDINSLEGLTEAYKLR
AARLVEIAAKNLQTHVSHRKSKEVAWNLTSVDLVR
ASEAHCHYVVVKVFSDKLPKIQDKAVQAVLRNLCL
LYSLYGISQKGGDFLEGSIITGAQLSQVNARILEL
LTLIRPNAVALVDAFDFKDMTLGSVLGRYDGNVYE
NLFEWAKKSPLNKTEVHESYHKHLKPLQSKL
ACO5 Protein
(Glycine max)
(SEQ ID NO: 14)
MEDGVDHLAFERNKAQFDVEDMKIIWAGSRQDFEL
SDRISRLVASDPAFRKDDRTRLIGRLFKNTLRKAA
YAWKRINELRLNEQEAYKLRSFVDQPAFTDLHWGM
FVPAIQGQGTDEQQQKWLPLAYGMQIIGCYAQTEL
GHGSNVQGLETTATFDPKTDEFVIHSPTLTSSKWW
PGGLGKISTHAVAYARLIIGGEDHGVHGFIVQLRS
LDDHLPLPGITIGDIGMKFGNAAYNTMDNGVLRFD
HVRIPRNQMLMRVSQVTREGRYVSSNVPRQLVYGT
MVNVRQKIVADASVALSRAVCIATRYSAVRRQFGS
HNGGLETQVIDYKTQQARLFPLLASAYAFRFVGGW
LKWLYMDVTERLQANDFSTLPEAHACTAGLKSLTT
TATADGIEECRKLCGGHGYLCSSGLPELFAVYVPA
CTYEGDNVVLLLQVARHLMKTVSQLGSGNKPVGTT
AYMARVEQLMQYHSDVEKAEDWLKPNVVLEAFEAR
ASRMSVACAQNLSKFANPEEGFQELAADLVDAAVA
HCQLIVVSKFIEKLQQDIPGKGVKKQLEVLCSIYA
LFLLHKHLGDFLSTGCINPKQGSLASEQLRNLYSQ
VRPNAIALVDAFNYTDHYLGSILGRYDGNVYPKMN
EEAWKDPLNDSVVPDGFKEYIQPMLKQQLRNARL
ACO6 Protein
(Glycine Max)
(SEQ ID NO: 15)
MEGMVDHLAFERNNSQFDVDEMKIVWAGSRHAFEV
SDKMARLVASDPAFRKDDRVVLDRKALFKNTLRKA
AYAWKRBELRLSEEEAAMLRSFVDQPAFTDLHWGM
FVPAIKGQGTEEQQKKWLPLAHKMQIIGCYAQTEL
GHGSNVQGLETTATFDPRTDEFVIHSPTLTSSKWW
PGGLGKVSTHAVVYARLITDGQDHGVHGFIVQLRS
LDDHLPLPGITVGDIGMKFGNGAYNSMDNGMLRFD
HVRIPRNQMLMRVSQVTREGKYVQSSVPRQLVYGT
MVYVRQTIVSDASVALSRAVCIATRYSAVRRQFGS
KEGGLETQVIDYKTQQARLFPLLASAYAFRFVGEW
LKWLYMDVMKRLQASDFSTLPEAHACTAGLKSLTT
SATADGIEECRKLCGGHGYLCSSGLPELFAVYIPT
CTYEGDNTVLLLQVARHLIKTISQLGSRNKPVGTT
SYIGRVEQLMQYRSDVQKVEDWLKPNAVLGAFEAR
AAKKVVACAQNLSKFTNPEEGFQELSVDLVEAAVA
HCQLIVVSKFIEKLQQDIPGKGVKQQLELLCSIYA
LFLLHKHLGDFLATGCITPKQGSLANELLRSLYSQ
VRPNAIALVDAFNYTDHYLGSVLGRYDGDVYPKLY
EEAWKDPLNDSVVPDGFQEYIRPMLKQQLRNARL
ACO7 Protein
(Source Beuvaria bassiana)
(SEQ ID NO: 16)
MSFPDNLKPKEPSGSSLLEKERRQSPVDVDALGKH
IFAGTSFLERQARVLRAIEQEPLFDKSRQQQLSRV
ERVKLGLARGKLMRRLQDRHGWDMDDYHMAAYLVG
EQSPYRLHVGMFRTTVEEQSSDAQRAYWMPRVNGW
EVSGAYSQTELGHGSNVRGVELEARWDPAAREFVV
HSPTLTAAKWWNGSLGRTANHAILMAQLMVPDPKR
EGQYISHGPQAFIAQIRDLKTNLPLEGVVIGDIGV
KIGFTSMDNGYMLFNQFRIPHSALLSRYVQLDPET
GVFSKSPNPALAYGTMTSIRTMLVEEAGTHLARAV
TIAIRYTAIRQQFRDKDSQDPSSAELQVLDYPTVQ
VRLFPLLAAAFALQYTGKVMRQDYAKTRGEVEKGN
LEGLAVMHSNSSGLKSLSTEITNAGIETCRRAMGG
HGYGSGSGLVEMQKDYQAKPILEGDNWMITQQTSS
FLIKRMTAAAKTRNEPPKDQIDAQLKTFLHQKDKG
RTFDILNSDSDIEESFKWRAASMTYDAYEARVIKK
KRHNDLLIQFHKLSHAHSQSIMVSSFLTTLTSSND
LAHETKEIVFDLYRLFAYTTIQAESYEFLRCGAAS
SKDLDALPERIQALLTRIRPHAVKLVDAWKIPDYL
LDSALGRYDGNVYEDLFNRAHRLNPLNDIVFNPDY
KDDEIVKGSGERKPLSPKL
ACO8 Protein
(Source Aspergillus nidulans)
(SEQ ID NO: 17)
MPNPPPAWVQALKPASPQGTELLTQERAQSNIDVD
TLGDLLHTKEALKKQDEILSVLKSEKVFDKSRNHV
LGRTEKIQLALARGKRLQQLKKAHNWSDEDVHVAN
DLVSEPTPYGLHASMFLVTLREQGTPEQHKLFYER
ARNYEIIGCYAQTELGHGSNVRGLETTATWDPSDQ
TFIIHSPTLTASKWWIGSLGRTANHAVVMAQLYIG
GKNYGPHPFVVQIRDMETHQPLENVYVGDIGPKFG
YNTMDNGFLLFNKLKIPHVNMLARFAQVDKATNKY
IRPASPSLMYGTMTWVRSNIVLQAGGVLARGVTIA
VRYCAVRRQFQDRDAKANAEENQVLNYKMVQIRLL
PLLAAMYALHFTGRGMMRLYEENQERMKAAAQADQ
EKRGAGPEQLRAGSDLLADLHATSCGLKALASTTA
GEGLEVCRRACGGHGYSNYSGIGPWYADYLPTLTW
EGDNYMLTQQVARYLLKSARAVLAGKGTANDTSRI
LQAYLARRDKGASFDILGNDADIVAAFAWRTAHLT
FETLKYRDVEKRSWNSLLINFWRLSTALSQYLVVK
NFYEAVNSPEIRSSLDKDTASTLRSLFRLHALHTL
DREASEFFSSAAVTVRQIGLTQTSEVPKLLDEIRP
HAVRLVDSWKIPDWQLDSALGRSDGDVYPDLFKRA
SMQNPVNDLVFDPYPWNENVLKNAGEIKSKL
ACO9 Protein
(Source Arabidopsis thaliana)
(SEQ ID NO: 18)
MESRREKNPMTEEESDGLIAARRIQRLSLHLSPSL
TPSPSLPLVQTETCSARSKKLDVNGEALSLYWIRG
KHIDIQEKIFDFFNSRPDLQTPIEISKDDHRELCM
NQLIGLVREAGVRPFRYVADDPEKYFAIMEAVGSV
DMSLGIKMGVQYSLWGGSVINLGTKKHRDKYFDGI
DNLDYTGCFAMTELHEGSNVQGLQTTATFDPLKDE
FVIDTPNDGAIKWWIGNAAVHGKFATVFARLILPT
HDSKGVSDMGVHAFIVPIRDMKTHQTLPGVEIQDC
GHKVGLNGVDNGALRFRSVRIPRDNLLNRFGDVSR
DGTYTSSLPTINKRFGATLGELVGGRVGLAYASVG
VLKISATIAIRYSLLRQQFGPPKQPEVSILDYQSQ
QHKLMPMLASTYAYHFATVYLVEKYSEMKKTHDEQ
LVADVHALSAGLKSYVTSYTAKALSVCREACGGHG
YAAVNRFGSLRNDHDIFQTFEGDNTVLLQQVAADL
LKRYKEKFQGGTLTVTWSYLRESMNTYLSQPNPVT
ARWEGEDHLRDPKFQLDAFRYRTSRLLQNVAARLQ
KHSKTLGGFGAWNRCLNHLLTLAESHIETVILAKF
IEAVKNCPDPSAKAALKLACDLYALDRIWKDIGTY
RNVDYVAPNKAKVCFLV
ACO10 Protein
(Source Arabidopsis thaliana)
(SEQ ID NO: 19)
MESRREKNPMTEEESDGLIAARRIQRLSLHLSPSL
TPSPSLPLVQTETCSARSKKLDVNGEALSLYWIRG
KHIDIQEKIFDFFNSRPDLQTPIEISKDDHRELCM
NQLIGLVREAGVRPFRYVADDPEKYFAIMEAVGSV
DMSLGIKMGVQYSLWGGSVINLGTKKHRDKYFDGI
DNLDYTGCFAMTELHHGSNVQGLQTTATFDPLKDE
FVIDTPNDGAIKWWIGNAAVHGKFATVFARLILPT
HDSKGVSDMGVHAFIVPIRDMKTHQTLPGVEIQDC
GHKVGLNGVDNGALRFRSVRIPRDNLLNRFGDVSR
DGTYTSSLPTINKRFGATLGELVGGRVGLAYASVG
VLKISATIAIRYSLLRQQFGPPKQPEVSILDYQSQ
QHKLMPMLASTYAYHFATVYLVEKYSEMKKTHDEQ
LVADVHALSAGLKSYVTSYTAKALSVCREACGGHG
YAAVNRFGSLRNDHDIFQTFEGDNTVLLQQVAADL
LKRYKEKFQGGTLTVTWSYLRESMNTYLSQPNPVT
ARWEGEDHLRDPKFQLDAFRYRTSRLLQNVAARLQ
KHSKTLGGFGAWNRCLNHLLTLAESHIETVILAKF
IEAVKNCPDPSAKAALKLACDLYALDRIWKDIGTY
RNVDYVAPNKAKAIHKLTEYLSFQVRNVAKELVDA
FELPDHVTRAPIAMQSDAYSQYTQVVGF

Proteins involved in beta-oxidation that their encoding gene may be modified, disrupted or replaced to produce a short-chain fatty acid intermediate.

POX4 Acyl-CoA Oxidase (Source Candida viswanathii)
(SEQ ID NO: 20)
MTFTKKNVSVSQGPDPRTSIQTERANSKFDPVTMNYFLEGSKERSELMKSLAQQIERDPI
LFTDGSYYDLTKDQQRELTVLKINRLSRYREGDSVDTFNKRLSIMGVVDPQVATRIGVNL
GLFLSCISGNGTAEQFKYWAIDKGTHNIQGLYGCFGMTELGHGSNVAGVETTATFDKETD
EFVINTPHIGATKWWIGGAAHSATHCSVYARLVVDGKDYGVKTFVVPLRDSNHDLMPGVT
VGDIGAKMGRDGIDNGWIQFSNVRIPRFFMLQKFCKVSAEGEVVLPPLEQLSYSALLGGR
VMMVLDSYRMLARVSTIALRYAIGRRQFKGDNVDQNDPNALETQLIDYPLHQKRLFPYLA
AAYVVSTGALKVEHTIQSTLATLDAAVENNDTTAIFKSIDDMKSLFIDSGSLKATTTWLA
AEAIDQCRQACGGHGYSSYNGFAKAFNDWVVQCTWEGDNNVLSLSVGKPIIKQIIGIEDN
GKTVRGSTAFLNQVKDFTGSNASKVVLNNTSDLNDINKVIKSIEVAIIRLAHEAAISVRK
ESLDFAGAELVQISKLKAHHYLLTEFVKRVGEFEHKELVPFLNTIGRLYSATVVLDKFAG
VFLTFNVASPQAITDLASTQIPKLCAEVRPNVVAYTDSFQQSDMVINSAIGKYDGDVYEN
YFDLVKQLNPPKNTKAPYTAALEGMLNRPSLEARERYEKSDETAAILSKPOX5
POX5 Acyl-CoA Oxidase (Source Candida viswanathii)
(SEQ ID NO: 21)
MPTELQKERELTKFNPKELNYFLEGSQERSEIISNMVEQMQKDPILKVDASYYNLTKDQQ
REVTAKKIARLSRYFEHEYPDQQAQRLSILGVFDPQVFTRIGVNLGLFVSCVRGNGTNSQ
FFYWTINKGIDKLRGIYGCFGMTELAHGSNVQGIETTATFDEDTDEFVINTPHIGATKWW
IGGAAHSATHCSVYARLKVKGKDYGVKTFVVPLRDSNHDLEPGVTVGDIGAKMGRDGIDN
GWIQFSNVRIPRFFMLQKYCKVSRSGEVTMPPSEQLSYSALIGGRVTMMMDSYRMTSRFI
TIALRYAIHRRQFKKKDTDTIETKLIDYPLHQKRLFPFLAAAYLFSQGALYLEQTMNATN
DKLDEAVSAGEKEAIDAAIVESKKLFVASGCLKSTCTWLTAEAIDEARQACGGHGYSSYN
GFGKAYSDWVVQCTWEGDNNILAMNVAKPMVRDLLKEPEQKGLVLSSVADLDDPAKLVKA
FDHALSGLARDIGAVAEDKGFDITGPSLVLVSKLNAHRFLIDGFFKRITPEWSEVLRPLG
FLYADWILTNFGATFLQYGIITPDVSRKISSEHFPALCAKVRPNVVGLTDGFNLTDMMTN
AAIGRYDGNVYEHYFETVKALNPPENTKAPYSKALEDMLNRPDLEVRERGEKSEEAAEIL
SS
POX1 Acyl-CoA Oxidase (Source Yarrowia lipolytica)
(SEQ ID NO: 22)
MTTNTFTDPPVEMAKERGKTQFTVRDVTNFLNGGEEETQIVEKIMSSIERDPVLSVTADY
DCNLQQARKQTMERVAALSPYLVTDTEKLSLWRAQLHGMVDMSTRTRLSIHNNLFIGSIR
GSGTPEQFKYWVKKGAVAVKQFYGCFAMTELGHGSNLKGLETTATYDQDSDQFIINTPHI
GATKWWIGGAAHTSTHCVCFAKLIVHGKDYGTRNFVVPLRNVHDHSLKVGVSIGDIGKKM
GRDGVDNGWIQFTNVRIPRQNMLMRYAKVSDTGVVTKPALDQLTYGALIRGRVSMIADSF
HVSKRFLTIALRYACVRRQFGTSGDTKETKIIDYPYHQRRLLPLLAYCYAMKMGADEAQK
TWIETTDRILALNPNDPAQKNDLEKAVTDTKELFAASAGMKAFTTWGCAKIIDECRQACG
GHGYSGYNGFGQGYADWVVQCTWEGDNNVLCLSMGRGLVQSALQILAGKHVGASIQYVGD
KSKISQNGQGTPREQLLSPEFLVEAFRTASRNNILRTTDKYQELVKTLNPDQAFEELSQQ
RFQCARIHTRQHLISSFYARIATAKDDIKPHLLKLANLFALWSIEEDTGIFLRENILTPG
DIDLINSLVDELCVAVRDQVIGLTDAFGLSDFFINAPIGSYDGNVYEKYFAKVNQQNPAT
NPRPPYYESTLKPFLFREEEDDEICDLDE
POX2 Acyl-CoA Oxidase (Source Yarrowia lipolytica)
(SEQ ID NO: 23)
MNPNNTGTIEINGKEYNTFTEPPVAMAQERAKTSFPVREMTYFLDGGEKNTLKNEQIMEE
IERDPLFNNDNYYDLNKEQIRELTMERVAKLSLFVRDQPEDDIKKRFALIGIADMGTYTR
LGVHYGLFFGAVRGTGTAEQFGHWISKGAGDLRKFYGCFSMTELGHGSNLAGLETTAIYD
EETDEFIINTPHIAATKWWIGGAAHTATHTVVFARLIVKGKDYGVKTFVVQLRNINDHSL
KVGISIGDIGKKMGRDGIDNGWIQFTNVRIPRQNLLMKYTKVDREGNVTQPPLAQLTYGS
LITGRVSMASDSHQVGKRFITIALRYACIRRQFSTTPGQPETKIIDYPYHQRRLLPLLAY
VYALKMTADEVGALFSRTMLKMDDLKPDDKAGLNEVVSDVKELFSVSAGLKAFSTWACAD
VIDKTRQACGGHGYSGYNGFGQAYADWVVQCTWEGDNNILTLSAGRALIQSAVALRKGEP
VGNAVSYLKRYKDLANAKLNGRSLTDPKVLVEAWEVAAGNIINRATDQYEKLIGEGLNAD
QAFEVLSQQRFQAAKVHTRRHLIAAFFSRIDTEAGEAIKQPLLNLALLFALWSIEEDSGL
FLREGFLEPKDIDTVTELVNKYCTTVREEVIGYTDAFNLSDYFINAPIGCYDGDAYRHYF
QKVNEQNPARDPRPPYYASTLKPFLFREEEDDDICELDEE
POX3 Acyl-CoA Oxidase (Source Yarrowia lipolytica)
(SEQ ID NO: 24)
MISPNLTANVEIDGKQYNTFTEPPKALAGERAKVKFPIKDMTEFLHGGEENVTMIERLMT
ELERDPVLNVSGDYDMPKEQLRETAVARIAALSGHWKKDTEKEALLRSQLHGIVDMGTRI
RLGVHTGLFMGAIRGSGTKEQYDYWVRKGAADVKGFYGCFAMTELGHGSNVAGLETTATY
IQDTDEFIINTPNTGATKWWIGGAAHSATHTACFARLLVDGKDYGVKIFVVQLRDVSSHS
LMPGIALGDIGKKMGRDAIDNGWIQFTNVRIPRQNMLMKYAKVSSTGKVSQPPLAQLTYG
ALIGGRVTMIADSFFVSQRFITIALRYACVRRQFGTTPGQPETKIIDYPYHQRRLLPLLA
FTYAMKMAADQSQIQYDQTTDLLQTIDPKDKGALGKAIVDLKELFASSAGLKAFTTWTCA
NIIDQCRQACGGHGYSGYNGFGQAYADWVVQCTWEGDNNVLCLSMGRGLIQSCLGHRKGK
PLGSSVGYLANKGLEQATLSGRDLKDPKVLIEAWEKVANGAIQRATDKFVELTKGGLSPD
QAFEELSQQRFQCAKIHTRKHLVTAFYERINASAKADVKPYLINLANLFTLWSIEEDSGL
FLREGFLQPKDIDQVTELVNHYCKEVRDQVAGYTDAFGLSDWFINAPIGNYDGDVYKHYF
AKVNQQNPAQNPRPPYYESTLRPFLFREDEDDDICELDEE*
POX4 Acyl-CoA Oxidase (Source Yarrowia lipolytica)
(SEQ ID NO: 25)
MITPNPANDIVHDGKLYDTFTEPPKLMAQERAQLDFDPRDITYFLDGSKEETELLESLML
MYERDPLFNNQNEYDESFETLRERSVKRIFQLSKSIAMDPEPMSFRKIGFLGILDMGTYA
RLGVHYALFCNSIRGQGTPDQLMYWLDQGAMVIKGFYGCFAMTEMGHGSNLSRLETIATF
DKETDEFIINTPHVGATKWWIGGAAHTATHTLAFARLQVDGKDYGVKSFVVPLRNLDDHS
LRPGIATGDIGKKMGRDAVDNGWIQFTNVRVPRNYMLMKHTKVLRDGTVKQPPLAQLTYG
SLITGRVQMTTDSHNVSKKFLTIALRYATIRRQFSSTPGEPETRLIDYLYHQRRLLPLMA
YSYAMKLAGDHVRELFFASQEKAESLKEDDKAGVESYVQDIKELFSVSAGLKAATTWACA
DIIDKARQACGGHGYSAYNGFGQAFQDWVVQCTWEGDNTVLTLSAGRALIQSALVYRKEG
KLGNATKYLSRSKELANAKRNGRSLEDPKLLVEAWEAVSAGAINAATDAYEELSKQGVSV
DECFEQVSQERFQAARIHTRRALIEAFYSRIATADEKVKPHLIPLANLFALWSTEEDSAL
FLAEGYFEPEDIIEVTSLVNKYCGIVRKNVIGYTDAFNLSDYFINAAIGRYDGDVYKNYF
EKVKQQYPPEGGKPHYYEDVMKPFLHRERIPDVPMEPEDIQ
POX5 Acyl-CoA Oxidase (Source Yarrowia lipolytica)
(SEQ ID NO: 26)
MNNNPTNVILGGKEYDTFTEPPAQMELERAKTQFKVRDVTNFLTGSEQETLLTERIMREI
ERDPVLNVAGDYDADLPTKRRQAVERIGALARYLPKDSEKEAILRGQLHGIVDMGTRTRI
AVHYGLFMGAIRGSGTKEQYDYWVAKGAATLHKFYGCFAMTELGHGSNVAGLETTATLDK
DTDEFIINTPNSGATKWWIGGAAHSATHTACLARLIVDGKDYGVKIFIVQLRDLNSHSLL
NGIAIGDIGKKMGRDAIDNGWIQFTDVRIPRQNMLMRYDRVSRDGEVTTSELAQLTYGAL
LSGRVTMIAESHLLSARFLTIALRYACIRRQFGAVPDKPETKLIDYPYHQRRLLPLLAYT
YAMKMGADEAQQQYNSSFGALLKLNPVKDAEKFAVATADLKALFASSAGMKAFTTWAAAK
IIDECRQACGGHGYSGYNGFGQAYADWVVQCTWEGDNNVLCLSMGRSLIQSCIAMRKKKG
HVGKSVEYLQRRDELQNARVDNKPLTDPAVLITAWEKVACEAINRATDSFIKLTQEGLSP
DQAFEELSQQRFECARIHTRKHLITSFYARISKAKARVKPHLTVLANLFAVWSIEEDSGL
FLREGCFEPAEMDEITALVDELCCEAREQVIGFTDAFNLSDFFINAPIGRFDGDAYKHYM
DEVKAANNPRNTHAPYYETKLRPFLFRPDEDEEICDLDE
POX6 Acyl-CoA Oxidase (Source Yarrowia lipolytica)
(SEQ ID NO: 27)
MLSQQSLNTFTEPPVEMARERNQTSFNPRLLTYFLDGGEKNTLLMDRLMQEYERDPVFRN
EGDYDITDVAQSRELAFKRIAKLIEYVHTDDEETYLYRCMLLGQIDMGAFARYAIHHGVW
GGAIRGAGTPEQYEFWVKKGSLSVKKFYGSFSMTELGHGSNLVGLETTATLDKNADEFVI
NTPNVAATKWWIGGAADTATHTAVFARLIVDGEDHGVKTFVVQLRDVETHNLMPGIAIGD
CGKKMGRQGTDNGWIQFTHVRIPRQNMLMRYCHVDSDGNVTEPMMAQMAYGALLAGRVGM
AMDSYFTSRKFLTIALRYATIRRAFAAGGGQETKLIDYPYHQRRLLPLMAQTYAIKCTAD
KVRDQFVKVTDMLLNLDVSDQEAVPKAIAEAKELFSVSAGVKATTTWACAHTIDQCRQAC
GGHGYSAYNGFGRAYSDWVIQCTWEGDNNILCLSAGRALVQSNRAVRAGKPIGGPTAYLA
APAGSPKLAGRNLYDPKVMIGAWETVSRALINRTTDEFEVLAKKGLSTAQAYEELSQQRF
LCTRIHTRLYMVKNFYERIAEEGTEFTKEPLTRLANLYAFWSVEEEAGIFLREGYITPQE
LKYISAEIRKQLLEVRKDVIGYTDAFNVPDFFLNSAIGRADGDVYKNYFKVVNTQNPPQD
PRPPYYESVIRPFLFRKDEDEEICSLEDE
FOX2 Peroxisomal hydratase-dehydrogenase-epimerase
(Source Candida viswanathii)
(SEQ ID NO: 28)
MSPVDFKDKVVIITGAGGGLGKYYSLEFAKLGAKVVVNDLGGALNGQGGNSKAADVVVDE
IVKNGGVAVADYNNVLDGDKIVETAVKNFGTVHVIINNAGILRDASMKKMTEKDYKLVID
VHLNGAFAVTKAAWPYFQKQKYGRIVNTSSPAGLYGNFGQANYASAKSALLGFAETLAKE
GAKYNIKANAIAPLARSRMTESILPPPMLEKLGPEKVAPLVLYLSSAENELTGQFFEVAA
GFYAQIRWERSGGVLFKPDQSFTAEVVAKRFSEILDYDDSRKPEYLKNQYPFMLNDYATL
TNEARKLPANDASGAPTVSLKDKVVLITGAGAGLGKEYAKWFAKYGAKVVVNDFKDATKT
VDEIKAAGGEAWPDQHDVAKDSEAIIKNVIDKYGTIDILVNNAGILRDRSFAKMSKQEWD
SVQQVHLIGTFNLSRLAWPYFVEKQFGRIINITSTSGIYGNFGQANYSSSKAGILGLSKT
MAIEGAKNNIKVNIVAPHAETAMTLTIFREQDKNLYHADQVAPLLVYLGTDDVPVTGETF
EIGGGWIGNTRWQRAKGAVSHDEHTTVEFIKEHLNEITDFTTDTENPKSTTESSMAILSA
VGGDDDDDDEDEEEDEGDEEEDEEDEEEDDPVWRFDDRDVILYNIALGATTKQLKYVYEN
DSDFQVIPTFGHLITFNSGKSQNSFAKLLRNFNPMLLLHGEHYLKVHSWPPPTEGEIKTT
FEPIATTPKGTNVVIVHGSKSVDNKSGELIYSNEATYFIRNCQADNKVYADRPAFATNQF
LAPKRAPDYQVDVPVSEDLAALYRLSGDRNPLHIDPNFAKGAKFPKPILHGMCTYGLSAK
ALIDKEGMENEIKARFTGIVFPGETLRVLAWKESDDTIVFQTHVVDRGTIAINNAAIKLV
GDKAKI

Proteins with motifs or regions important for cellular localization are as follows.

OST1 (Source Candida viswanathii)
(SEQ ID NO: 29)
MMWKFLIAIGLIFSYCCNAQLLDSLSFDNNWVNTHYIRTIDLSKGFVKETDLIQIKNIND
KPQDEYYFVVNDGFDSIDELSIFSAFVGDQALEVEVDEVVPDKVFKLKLPVPIAPNSDLE
LRINFVYIDSLVSVPSKIAMDATQQLLYKTNKFPFSPYVTQEYTLALSGMSKGQEMDLHI
DVEDTPGLPDLKPRVESQVLKYGPIAEDIPAFALKPMGLMYDHNRPLTKAVSLNRSIWLP
ASDINKVSIEEYYELTNTGAELDKGFSRVDWMKGRFESTRNHWALSHLEIPLLERGFDDY
YYTDKVGVVSTHKIFKNHLLLQPRYPVFGGWKYNFTLGWSEELSKFLHKLHDNQDEYIIK
FPILNSLRDVTYQDVYLEFYLPENAEFQNISSPIAFESISIENELSYLDVSKGHTKITVH
YTNLFDDLHKLDVFVKYQYTQVAFIYKIAKISGFVFLGLVSYYLLGLLDLSI
GlU1 Glucoamylase (Source Saccharomyces fibuligera)
(SEQ ID NO: 30)
MKFGVLFSVFAAIVSALPLQEGPLNKRAYPSFEAYSNYKVDRTDLETFLDKQKEVSLYYL
LQNIAYPEGQFNNGVPGTVIASPSTSNPDYYYQWTRDSAITFLTVLSELEDNNFNTTLAK
AVEYYINTSYNLQRTSNPSGSFDDENHKGLGEPKFNTDGSAYTGAWGRPQNDGPALRAYA
ISRYLNDVNSLNEGKLVLTDSGDINFSSTEDIYKNIIKPDLEYVIGYWDSTGFDLWEENQ
GRHFFTSLVQQKALAYAVDIAKSFDDGDFANTLSSTASTLESYLSGSDGGFVNTDVNHIV
ENPDLLQQNSRQGLDSATYIGPLLTHDIGESSSTPFDVDNEYVLQSYYLLLEDNKDRYSV
NSAYSAGAAIGRYPEDVYNGDGSSEGNPWFLATAYAAQVPYKLAYDAKSASNDITINKIN
YDFFNKYIVDLSTINSAYQSSDSVTIKSGSDEFNTVADNLVTFGDSFLQVILDHINDDGS
LNEQLNRYTGYSTGAYSLTWSSGALLEAIRLRNKVKALA
SIAT1_RAT alpha-2,6-sialyltransferase (Source
Rat norvegicus)
(SEQ ID NO: 31)
MIHTNLKKKFSLFILVFLLFAVICVWKKGSDYEALTLQAKEFQMPKSQEKVAMGSASQVV
FSNSKQDPKEDIPILSYHRVTAKVKPQPSFQVWDKDSTYSKLNPRLLKIWRNYLNMNKYK
VSYKGPGPGVKFSVEALRCHLRDHVNVSMIEATDFPFNTTEWEGYLPKENFRTKVGPWQR
CAVVSSAGSLKNSQLGREIDNHDAVLRFNGAPTDNFQQDVGSKTTIRLMNSQLVTTEKRF
LKDSLYTEGILIVWDPSVYHADIPKWYQKPDYNFFETYKSYRRLNPSQPFYILKPQMPWE
LWDIIQEISADLIQPNPPSSGMLGIIIMMTLCDQVDIYEFLPSKRKTDVCYYHQKFFDSA
CTMGAYDPLLFEKNMVKHLNEGTDEDIYLFGKATLSGFRNIRC
B4galt1 Beta-1,4-galactosyltransferase 1 (Source
Homo sapiens)
(SEQ ID NO: 32)
MRLREPLLSGSAAMPGASLQRACRLLVAVCALHLGVTLVYYLAGRDLSRLPQLVGVSTPL
QGGSNSAAAIGQSSGELRTGGARPPPPLGASSQPRPGGDSSPVVDSGPGPASNLTSVPVP
HTTALSLPACPEESPLLVGPMLIEFNMPVDLELVAKQNPNVKMGGRYAPRDCVSPHKVAI
IIPFRNRQEHLKYWLYYLHPVLQRQQLDYGIYVINQISF
DOA4 Ubiquitin carboxyl-terminalhydrolase4
(Source Candida viswanathii)
(SEQ ID NO: 33)
MTLLLKPTSELDATSRKIIERIQSNSPTFQHLFDLLLNLLPFFDKTVSLLGSIGYCDYEV
AYVTYQTCIQVVGLMKPKTNSLNQDIFKGVQLQTRKRASTFKAILSYFAEPETQEEDPLL
NRFKSLSGGGSKTKSSQDEVFHEWITSSELQRELSSKKVLLIDFRPRKDYLNNHIKYKDL
VHIEPTQLETLLDSASDQDLETLVKKSAPYDQYHIFLERHKYDLIVVYNYNYGSESTDRL
LGIIDVVSKPNPFTKLITILMNNKYISSRLKVKPLFLSGGVLNWYKTFGIEYLERTLVQN
GVAHTSDNQYLKSFNDYVSTSKETPKTQVKTQNGDYIRPSQRKVNQFDPVPVKSGPTVFA
SAKVDLPPTPGSPAVSTPSPPRAPAPPTKTTSLTHVPEKEAKSPSPVTKEVTVSSKKSQF
LELYTTGLVNLGNSCYMNCVVQCLAAAPQLTSFFFPTITESFSDHSYRQHINSNNKLGTK
GELTTSFVELILNMLNNNGKAFSPTKFKRTMGSLSPSQQFLTYDQQDCIEFLNFLLDALH
EDLNNVTITDPSERKLITDLSPEQEKSRETLPVRLASTIEWERYLKLNFSVIVDYFQGQH
LSQLKCLECGFTSTTYNAFSILSLPIPQKLNNLGKVLLKDCLEEFVTTELLDDNNKWYCP
QCKRFTRLTKKIAITRLPQVLIVNFNRFKMTNTGGFNKLETFVTYPVNEELDMTPYWPDV
GSRINENSTMSIEMEQDLLQSFPIRNQTPPFKYKLFGVANHFGNLTTGHYTSYVYKHSDS
KKTRNWCYFDDSKITYNVSPSQVVNKNAYCLFFQRV
APR1 Vacuolar aspartic protease (Source
Candida viswanathii)
(SEQ ID NO: 34)
MQLSLSVLSTVATALLSLTTAVDAKSHNIKLSKLSNEETLDASTFQEYTSSLANKYMNLF
NAAHGNPTSFGLQHVLSNQEAEVPFVTPQKGGKYDAPLTNYLNAQYFTEIEIGTPGQPFK
VILDTGSSNLWVPSQDCTSLACFLHSKYDHDASSTYKANGSEFSIQYGSGSMEGYISQDI
LTIGDLVIPKQDFAEATSEPGLAFAFGKFDGILGLAYDSISVNHIVPPVYNAINQGLLDK
PQVSFYLGNTEKDENDGGLATFGGYDASLFQGKITWLPVRRKAYWEVSFEGIGLGDEYAE
LQKTGAAIDTGTSLITLPSSLAEIINAKIGATKSWSGQYQIDCAKRDELPDLTLTFAGHN
FTLTAHDYILEVSGSCISVFTPMDFPKPIGDLAIIGDAFLRKYYSIYDLDKNAVGLAPSK
A
PHO8 (Source Candida viswanathii)
(SEQ ID NO: 35)
MGITNETQALLGGDSLSCLNKKKSNTKRNLSYLLNIITVSIIAYLCFFATHNHHNDSGIP
KVDPHKKKNIIMMVTDGMGPASLSAARSFRQFRDKLAINDILTLDQYLIGSSRTRSSSSL
VTDSAAGATAFSCALKSYNGAIGVSPDKSPCGTILEALKLQGYYTGLVVTTRITDATPAA
FSAHVDYRFQEDLIAEHQLGEYPFGRAVDLILGGGRCHFLPTAQGGCRADDRNLIKESSD
TWQYVGDRQQFDQLKGGKNVSLPLLGLLANTDIPYAIDRDEKEYPSLAEQVKVALTALSD
ATKDSDQGFFLLIEGSRIDHAGHEINDPTAQVREVLAYDEAFGEVIKFIDSTDVETVATS
TSDHETGGLVVSRQVTPEYPDYIWYPEVLLNSTHSGDYLAHKIADYKNKDDTAKLTKFIK
HEILETDLGVTDYTDKDVQAILDKVNDPANLLYVLNDIVSFRAQIGWTTHGHSAVDVNIY
AHTNSPAIRAKLASAKAYHGLSGNHENIEIGAFMEEITGSNLSRVTELIKKTAHSPSLSK
KEFSVDEFHGNV
SNA4 (Source Saccharomyces cerevisiae)
(SEQ ID NO: 36)
MCCYCVCCTVSDFILYIVAFFFPPAAVLLRSGPCSSDFLLNVLLTLLGFLPGMLHAFYYI
TITSPLRNAEYVYYYQQGWVDSERNVPSNRPQNSQTPQNRPQQGSSARNVYPSVETPLLQ
GAAPHDNKQSLVESPPPYVP
OCH1 (Source Candida viswanathii)
(SEQ ID NO: 37)
MRLKDIKLILIGILTISVTYFLISSFSGPRAYTTSDPNSSKMQFLRALESHPNWKETGLN
FQPTKKLEVDDSSTPVRQQLAARFPYDPTQPFPKNIWQTWKVGIEDETFPKRYLKFQLSW
DTKNPEYKHHVIPDDQCDELVAQLFEDVPDVARAYKVMPKSILKADFFRYLILFARGGVY
TDIDTVGLKPIDTWMSNMELLWGEPNRAGLVVGIEADPDRPDWADWYARRIQFCQWTIQL
KKGHPMLRELITKITDITLTREKRNELKKVLGKDEGGDIMNWTGPGIFTDTVFSYMNAIL
QAPEVITGKYKWDNIVDWKVFTGMQMPIAIDDVLVLPITSFSPDVSQMGSKSSTDPMAYA
KHMFLGSWKDDGMPEME

Proteins for use in the cannabinoid pathway are as follows.

HXS1 Protein (Source Cannabis sativa)
(SEQ ID NO: 38)
MGKNYKSLDSVVASDFIALGITSEVAETLHGRLAEIVCNYGAATPQTWINIANHILSPDL
PFSLHQMLFYGCYKDFGPAPPAWIPDPEKVKSTNLGALLEKRGKEFLGVKYKDPISSFSH
FQEFSVRNPEVYWRTVLMDEMKISFSKDPECILRRDDINNPGGSEWLPGGYLNSAKNCLN
VNSNKKLNDTMIVWRDEGNDDLPLNKLTLDQLRKRVWLVGYALEEMGLEKGCAIAIDMPM
HVDAVVIYLAIVLAGYVVVSIADSFSAPEISTRLRLSKAKAIFTQDHIIRGKKRIPLYSR
VVEAKSPMAIVIPCSGSNIGAELRDGDISWDYFLERAKEFKNCEFTAREQPVDAYTNILF
SSGTTGEPKAIPWTQATPLKAAADGWSHLDIRKGDVIVWPTNLGWMMGPWLVYASLLNGA
SIALYNGSPLVSGFAKFVQDAKVTMLGVVPSIVRSWKSTNCVSGYDWSTIRCFSSSGEAS
NVDEYLWLMGRANYKPVIEMCGGTEIGGAFSAGSFLQAQSLSSFSSQCMGCTLYILDKNG
YPMPKNKPGIGELALGPVMFGASKTLLNGNHHDVYFKGMPTLNGEVLRRHGDIFELTSNG
YYHAHGRADDTMNIGGIKISSIEIERVCNEVDDRVFETTAIGVPPLGGGPEQLVIFFVLK
DSNDTTIDLNQLRLSFNLGLQKKLNPLFKVTRVVPLSSLPRTATNKIMRRVLRQQFSHFE
TKS1 Protein (Source Cannabis sativa)
(SEQ ID NO: 39)
MNHLRAEGPASVLAIGTANPENILIQDEFPDYYFRVTKSEHMTQLKEKFRKICDKSMIRK
RNCFLNEEHLKQNPRLVEHEMQTLDARQDMLVVEVPKLGKDACAKAIKEWGQPKSKITHL
IFTSASTTDMPGADYHCAKLLGLSPSVKRVMMYQLGCYGGGTVLRIAKDIAENNKGARVL
AVCCDIMACLFRGPSDSDLELLVGQAIFGDGAAAVIVGAEPDESVGERPIFELVSTGQTI
LPNSEGTIGGHIREAGLIFDLHKDVPMLISNNIEKCLIEAFTPIGISDWNSIFWITHPGG
KAILDKVEEKLDLKKEKFVDSRHVLSEHGNIVISSSTVLFVMDELRKRSLEEGKSTTGDG
FEWGVLFGFGPGLTVERVVVRSVPIKY
TKS1P Protein (Source Cannabis sativa)
(SEQ ID NO: 40)
MNHLRAEGPASVLAIGTANPENILIQDEFPDYYFRVTKSEHMTQLKEKFRKICDKSMIRK
RNCFLNEEHLKQNPRLVEHEMQTLDARQDMLVVEVPKLGKDACAKAIKEWGQPKSKITHL
IFTSASTTDMPGADYHCAKLLGLSPSVKRVMMYQLGCYGGGTVLRIAKDIAENNKGARVL
AVCCDIMACLFRGPSDSDLELLVGQAIFGDGAAAVIVGAEPDESVGERPIFELVSTGQTI
LPNSEGTIGGHIREAGLIFDLHKDVPMLISNNIEKCLIEAFTPIGISDWNSIFWITHPGG
KAILDKVEEKLDLKKEKFVDSRHVLSEHGNMSSSTVLFVMDELRKRSLEEGKSTTGDGFE
WGVLFGFGPGLTVERVVVRSVPIKYGRRAKL
OAC1 Protein (Source Cannabis sativa)
(SEQ ID NO: 41)
MAVKHLIVLKFKDEITEAQKEEFFKTYVNLVNIIPAMKDVYWGKDVTQKNKEEGYTHIVE
VTFESVETIQDYIIHPAHVGFGDVYRSFWEKLLIFDYTPRKLKPK
OAC1P Protein (Source Cannabis sativa)
(SEQ ID NO: 42)
MAVKHLIVLKFKDEITEAQKEEFFKTYVNLVNIIPAMKDVYWGKDVTQKNKEEGYTHIVE
VTFESVETIQDYIIHPAHVGFGDVYRSFWEKLLIFDYTPRKLKPKGRRAKL
PTS1 Protein (Source Cannabis sativa)
(SEQ ID NO: 43)
MGLSSVCTFSFQTNYHTLLNPHNNNPKTSLLCYRHPKTPIKYSYNNFPSKHTKS1FHLQN
KCSESLSIAKNSIRAATTNQTEPPESDNHSVATKILNFGKACWKLQRPYTHAFTSCACGL
FGKELLHNTNLISWSLMFKAFFFLVAILCIASFTTTINQIYDLHIDRINKPDLPLASGEI
SVNTAWIMSIIVALFGLITTIKMKGGPLYIFGYCFGIFGGIVYSVPPFRWKQNPSTAFLL
NFLAHIITNFTFYYASRAALGLPFELRPSFTFLLAFMKSMGSALALIKDASDVEGDTKFG
ISTLASKYGSRNLTLFCSGIVLLSYVAAILAGIIWPQAFNSNVMLLSHAILAFWLILQTR
DFALTNYDPEAGRRFYEFMWKLYYAEYLVYVFI
PTS1dN (Source Cannabis sativa)
(SEQ ID NO: 44)
MAATTNQTEPPESDNHSVATKILNFGKACWKLQRPYTIIAFTSCACGLFGKELLHNTNLI
SWSLMFKAFFFLVAILCIASFTTTINQIYDLHIDRINKPDLPLASGEISVNTAWIMSIIV
ALFGLIITIKMKGGPLYIFGYCFGIFGGIVYSVPPFRWKQNPSTAFLLNFLAHIITNFTF
YYASRAALGLPFELRPSFTFLLAFMKSMGSALALIKDASDVEGDTKFGISTLASKYGSRN
LTLFCSGIVLLSYVAAILAGIIWPQAFNSNVMLLSHAILAFWLILQTRDFALTNYDPEAG
RRFYEFMWKLYYAEYLVYVFI
PTS2 Protein (Source Cannabis sativa)
(SEQ ID NO: 45)
MGLSLVCTFSFQTNYHTLLNPHNKNPKNSLLSYQHPKTPIIKSSYDNFPSKYCLTKNFHL
LGLNSHNRISSQSRSIRAGSDQIEGSPHHESDNSIATKILNFGHTCWKLQRPYVVKGMIS
IACGLFGRELFNNRHLFSWGLMWKAFFALVPILSFNFFAAIMNQIYDVDIDRINKPDLPL
VSGEMSIETAWILSIIVALTGLIVTIKLKSAPLFVFIYIFGIFAGFAYSVPPIRWKQYPF
TNFLITISSHVGLAFTSYSATTSALGLPFVWRPAFSFITAFMTVMGMTIAFAKDISDIEG
DAKYGVSTVATKLGARNMTFVVSGVLLLNYLVSISIGIIWPQVFKSNIMILSHAILAFCL
IFQTRELALANYASAPSRQFFEFIWLLYYAEYFVYVFI
PTS3 Protein (Source Cannabis sativa)
(SEQ ID NO: 46)
MGLSSVCTFSFQTNYHTLLNPHNNNPKTSLLYRHPKTPIKYSYNNFPSKHCSTKSFHLQN
KCSESLSIAKNSIRAATTNQTEPPESDNHSVATKILNFGKACWKLQRPYTIIAFTSCACG
LFGKELLHNTNLISWSLMFKAFFFLVAVLCIASFTTTINQIYDLHIDRINKPDLPLASGE
ISVNTAWIMSIIVALFGLIITIKMKGGPLYIFGYCFGIFGGTVYSVPPFRWKQNPSTAFL
LNFLAHIITNFTFYHASRAALGLPFELRPSFTFLLAFMKSMGSALALIKDASDVEGDTKF
GISTLASKYGSRNLTLFCSGIVLLSYVAAILAGIIWPQAFNSNVMLLSHAILAFWLILQT
RDFALTNYDPEAGRRFYEFMWKLYYAEYLVYVFI
PTS4 Protein (Source Cannabis sativa)
(SEQ ID NO: 47)
MELSSICNFSFQTNYHTLLNPHNKNPKSSLLSHQHPKTPIITSSYNNFPSNYCSNKNFHL
QNRCSKSLLIAKNSIRTDTANQTEPPESNTKYSVVTKILSFGHTCWKLQRPYTFIGVISC
ACGLFGRELFHNTNLLSWSLMLKAFSSLMVILSVNLCTNIINQITDLDIDRINKPDLPLA
SGEMSIETAWIMSIIVALTGLILTIKLNCGPLFISLYCVSILVGALYSVPPFRWKQNPNT
AFSSYFMGLVIVNFTCYYASRAAFGLPFEMSPPFTFILAFVKSMGSALFLCKDVSDIEGD
SKHGISTLATRYGAKNITFLCSGIVLLTYVSAILAAIIWPQAFKSNVMLLSHATLAFWLI
FQTREFALTNYNPEAGRKFYEFMWKLHYAEYLVYVFI
PTS5 Protein (Source Cannabis sativa)
(SEQ ID NO: 48)
MVFSSVCSFPSSLGTNFKLVPRSNFKASSSHYHEINNFINNKPIKFSYFSSRLYCSAKPI
VHRENKFTKSFSLSHLQRKSSIKAHGEIEADGSNGTSEFNVMKSGNAIWRFVRPYAAKGV
LFNSAAMFAKELVGNLNLFSWPLMFKILSFTLVILCIFVSTSGINQIYDLDIDRLNKPNL
PVASGEISVELAWLLTIVCTISGLTLTIITNSGPFFPFLYSASIFFGFLYSAPPFRWKKN
PFTACFCNVMLYVGTSVGVYYACKASLGLPANWSPAFCLLFWFISLLSIPISIAKDLSDI
EGDRKFGIITFSTKFGAKPIAYICHGLMLLNYVSVMAAAIIWPQFFNSSVILLSHAFMAI
WVLYQAWILEKSNYATETCQKYYIFLWIIFSLEHAFYLFM
PTS6 Protein (Source Cannabis sativa)
(SEQ ID NO: 49)
MELSLSLGGPTIFPRYRASYTSTKLTTHFSNFPSKFSTKNFHQTLSFYGPTRGSKSLLNT
HQWRNSIRACAEAGAAGSNPVLNKVSDFRDACWRFLRPHTIRGTTLGSIALVARALIENP
NLIKWSLLLKAFSGLLALICGNGYIVGINQIYDIGIDKVNKPYLPIAAGDLSVQSAWYLV
ILFAVAGLLTVGFNFGPFITSLYCLGLVLGTIYSVPPFRMKRFPVAAFLIIATVRGFLLN
FGVYYATRAALGLTFEWSSAVAFITTFVTLFALVIAITKDLPDVEGDRKFQISTFATKLG
VRNIAYLGSGLLLLNYIGAIAAAIYMPQAFKRNLMLPIHTILALSLVFQAWVLEQANYTK
EAIAGFYRFIWNLFYVEYIIFPFI
PTS7 Protein (Source Cannabis sativa)
(SEQ ID NO: 50)
MAIALWLPRISRSTTRRFLKPSSSLTLFSVSHSHNYIVTSNRSPIPRLFTVPNQSHGREW
VSVSEVRLGYVSHISTAGKSDENRSRDAQVADVSWIDLYLPRQIHPYVRLARLDKPIGTW
LLAWPCMWSISLAANPGHLPDIKMMTLFGCGALLLRGAGCTINDLLDRDIDTMVERTKLR
PVASGIITPFQGICFLGFQLLLGLGILLQLNNYSRILGASSLLLVFSYPLMKRLTFWPQA
YLGLTFNWGALLGWAAVKGNIDPAIVLPLYASGVFWTLVYDTIYAHQDKEDDVRVGIKST
ALRFGDLTKQWNMGFGAACISSLALSGYNAEIGWPFYASLVAASGQLAWQISTVDLSSRD
DCNKKFVSNKWFGAIIFSGIVLARISS
PTS8 Protein (Source Cannabis sativa)
(SEQ ID NO: 51)
MSEAADVERVYAAMEEAAGLLGVACARDKIYPLLSTFQDTLVEGGSVVVFSMASGRHSTE
LDFSISVPTSHGDPYATVVEKGLFPATGHPVDDLLADTQKHLPVSMFAIDGEVTGGFKKT
YAFFPTDNMPGVAELSAIPSMPPAVAENAELFARYGLDKVQMTSMDYKKRQVNLYFSELS
AQTLEAESVLALVRELGLHVPNELGLKFCKRSFSVYPTLNWETGKIDRLCFAVISNDPTL
VPSSDEGDIEKFHNYATKAPYAYVGEKRTLVYGLTLSPKEEYYKLGAYYHITDVQRGLLK
AFDSLEDG
PTS9 Protein (ScNphB) (Source Streptomyces)
(SEQ ID NO: 52)
MSEAADVERVYAAMEEAAGLLGVACARDKIYPLLSTFQDTLVEGGSVVVFSMASGRHSTE
LDFSISVPTSHGDPYATVVEKGLFPATGHPVDDLLADTQKHLPVSMFAIDGEVTGGFKKT
YAFFPTDNMPGVAELSAIPSMPPAVAENAELFARYGLDKVQMTSMDYKKRQVNLYFSELS
AQTLEAESVLALVRELGLHVPNELGLKFCKRSFSVYPTLNWETGKIDRLCFAVISNDPTL
VPSSDEGDIEKFHNYATKAPYAYVGEKRTLVYGLTLSPKEEYYKLGAYYHITDVQRGLLK
AFDSLEDGMKCSTFSFWFVCKIIFFFFSFNIQTSIANPRENFLKCFSQYIPNNATNLKLV
YTQNNPLYMSVLNSTIHNLRFTSDTTPKPLVIVTPSHVSHIQGTILCSKKVGLQIRTRSG
GHDSEGMSYISQVPFVIVDLRNMRSIKIDVHSQTAWVEAGATLGEVYYWVNEKNENLSLA
AGYCPTVCAGGHFGGGGYGPLMRNYGLAADNIIDAHLVNVHGKVLDRKSMGEDLFWALRG
GGAESFGIIVAWKIRLVAVPKSTMFSVKKIMEIHELVKLVNKWQNIAYKYDKDLLLMTHF
ITRNITDNQGKNKTAIHTYFSSVFLGGVDSLVDLMNKSFPELGIKKTDCRQLSWIDTIIF
YSGVVNYDTDNFNKEILLDRSAGQNGAFKIKLDYVKKPIPESVFVQILEKLYEEDIGAGM
YALYPYGGIMDEISESAIPFPHRAGILYELWYICSWEKQEDNEKHLNWIRNIYNFMTPYV
SKNPRLAYLNYRDLDIGINDPKNPNNYTQARIWGEKYFGKNFDRLVKVKTLVDPNNFFRN
EQSIPPLPRHRH
CBD1dNS1 Protein (Source Cannabis sativa)
(SEQ ID NO: 53)
MFLKHIFVALAFALLADATPAQKRSPGFVALDFDIVKVQKNVTANDDAAAIVAKRQTNPR
ENFLKCFSQYIPNNATNLKLVYTQNNPLYMSVLNSTIHNLRFTSDTTPKPLVIVTPSHVS
HIQGTILCSKKVGLQIRTRSGGHDSEGMSYISQVPFVIVDLRNMRSIKIDVHSQTAWVEA
GATLGEVYYWVNEKNENLSLAAGYCPTVCAGGHFGGGGYGPLMRNYGLAADNIIDAHLVN
VHGKVLDRKSMGEDLFWALRGGGAESFGIIVAWKIRLVAVPKSTMFSVKKIMEIHELVKL
VNKWQNIAYKYDKDLLLMTHFITRNITDNQGKNKTAIHTYFSSVFLGGVDSLVDLMNKSF
PELGIKKTDCRQLSWIDTIIFYSGVVNYDTDNFNKEILLDRSAGQNGAFKIKLDYVKKPI
PESVFVQILEKLYEEDIGAGMYALYPYGGIMDEISESAIPFPHRAGILYELWYICSWEKQ
EDNEKHLNWIRNIYNFMTPYVSKNPRLAYLNYRDLDIGINDPKNPNNYTQARIWGEKYFG
KNFDRLVKVKTLVDPNNFFRNEQSIPPLPRHRH
CBD1dNS2 Protein (Source Cannabis sativa)
(SEQ ID NO: 54)
MQLSLSVLSTVATALLSLTTAVDAKSHNPRENFLKCFSQYIPNNATNLKLVYTQNNPLYM
SVLNSTIHNLRFTSDTTPKPLVIVTPSHVSHIQGTILCSKKVGLQIRTRSGGHDSEGMSY
ISQVPFVIVDLRNMRSIKIDVHSQTAWVEAGATLGEVYYWVNEKNENLSLAAGYCPTVCA
GGHFGGGGYGPLMRNYGLAADNIIDAHLVNVHGKVLDRKSMGEDLFWALRGGGAESFGII
VAWKIRLVAVPKSTMFSVKKIMEIHELVKLVNKWQNIAYKYDKDLLLMTHFITRNITDNQ
GKNKTAIHTYFSSVFLGGVDSLVDLMNKSFPELGIKKTDCRQLSWIDTIIFYSGVVNYDT
DNFNKEILLDRSAGQNGAFKIKLDYVKKPIPESVFVQILEKLYEEDIGAGMYALYPYGGI
MDEISESAIPFPHRAGILYELWYICSWEKQEDNEKHLNWIRNIYNFMTPYVSKNPRLAYL
NYRDLDIGINDPKNPNNYTQARIWGEKYFGKNFDRLVKVKTLVDPNNFFRNEQSIPPLPR
HRH
CBD1dNV1 Protein (Source Cannabis sativa)
(SEQ ID NO: 55)
MQLSLSVLSTVATALLSLTTAVDAKSHNIKLSKLSNEETLDASTFQEYTSSLANKYMNLF
NAAHGNPTSFGLQHVLSNQEAEVPFVTPQKGGNPRENFLKCFSQYIPNNATNLKLVYTQN
NPLYMSVLNSTIHNLRFTSDTTPKPLVIVTPSHVSHIQGTILCSKKVGLQIRTRSGGHDS
EGMSYISQVPFVIVDLRNMRSIKIDVHSQTAWVEAGATLGEVYYWVNEKNENLSLAAGYC
PTVCAGGHFGGGGYGPLMRNYGLAADNIIDAHLVNVHGKVLDRKSMGEDLFWALRGGGAE
SFGIIVAWKIRLVAVPKSTMFSVKKIMEIHELVKLVNKWQNIAYKYDKDLLLMTHFITRN
ITDNQGKNKTAIHTYFSSVFLGGVDSLVDLMNKSFPELGIKKTDCRQLSWIDTIIFYSGV
VNYDTDNFNKEILLDRSAGQNGAFKIKLDYVKKPIPESVFVQILEKLYEEDIGAGMYALY
PYGGIMDEISESAIPFPHRAGILYELWYICSWEKQEDNEKHLNWIRNIYNFMTPYVSKNP
RLAYLNYRDLDIGINDPKNPNNYTQARIWGEKYFGKNFDRLVKVKTLVDPNNFFRNEQSI
PPLPRHRH
CBD1dNP1 Protein (Source Cannabis sativa)
(SEQ ID NO: 56)
MNPRENFLKCFSQYIPNNATNLKLVYTQNNPLYMSVLNSTIHNLRFTSDTTPKPLVIVTP
SHVSHIQGTILCSKKVGLQIRTRSGGHDSEGMSYISQVPFVIVDLRNMRSIKIDVHSQTA
WVEAGATLGEVYYWVNEKNENLSLAAGYCPTVCAGGHFGGGGYGPLMRNYGLAADNBDAH
LVNVHGKVLDRKSMGEDLFWALRGGGAESFGIIVAWKIRLVAVPKSTMFSVKKIMEIHEL
VKLVNKWQNIAYKYDKDLLLMTHFITRNITDNQGKNKTAIHTYFSSVFLGGVDSLVDLMN
KSFPELGIKKTDCRQLSWIDTIIFYSGVVNYDTDNFNKEILLDRSAGQNGAFKIKLDYVK
KPIPESVFVQILEKLYEEDIGAGMYALYPYGGIMDEISESAIPFPHRAGILYELWYICSW
EKQEDNEKHLNWIRNIYNFMTPYVSKNPRLAYLNYRDLDIGINDPKNPNNYTQARIWGEK
YFGKNFDRLVKVKTLVDPNNFFRNEQSIPPLPRHRHGRRAKL
THC1 Protein (Source Cannabis sativa)
(SEQ ID NO: 57)
MNCSAFSFWFVCKIIFFFLSFNIQISIANPQENFLKCFSEYIPNNPANPKFIYTQHDQLY
MSVLNSTIQNLRFTSDTTPKPLVIVTPSNVSHIQASILCSKKVGLQIRTRSGGHDAEGMS
YISQVPFVVVDLRNMEISIKIDVHSQTAWVEAGATLGEVYYWINEKNENFSFPGGYCPTV
GVGGHFSGGGYGALMRNYGLAADNIIDAHLVNVDGKVLDRKSMGEDLFWAIRGGGGENFG
IIAAWKIKLVAVPSKSTIFSVKKNMEIHGLVKLFNKWQNIAYKYDKDLVLMTHFITKNIT
DNHGKNKTTVHGYFSSIFHGGVDSLVDLMNKSFPELGIKKTDCKEFSWIDTTIFYSGVVN
FNTANFKKEILLDRSAGKKTAFSIKLDYVKKPIPETAMVKILEKLYEEDVGVGMYVLYPY
GGIMEEISESAIPFPHRAGIMYELWYTASWEKQEDNEKHINWVRSVYNFTTPYVSQNPRL
AYLNYRDLDLGKTNPESPNNYTQARIWGEKYFGKNFNRLVKVKTKADPNNFFRNEQSIPP
LPPHHH
THC1dNS1 Protein (Source Cannabis sativa)
(SEQ ID NO: 58)
MFLKHIFVALAFALLADATPAQKRSPGFVALDFDIVKVQKNVTANDDAAAIVAKRQTNPQ
ENFLKCFSEYIPNNPANPKFIYTQHDQLYMSVLNSTIQNLRFTSDTTPKPLVIVTPSNVS
HIQASILCSKKVGLQIRTRSGGHDAEGMSYISQVPFVVVDLRNIVIHSIKIDVHSQTAWV
EAGATLGEVYYWINEKNENFSFPGGYCPTVGVGGHFSGGGYGALMRNYGLAADNIIDAHL
VNVDGKVLDRKSMGEDLFWAIRGGGGENFGIIAAWKIKLVAVPSKSTIFSVKKNMEIHGL
VKLFNKWQNIAYKYDKDLVLMTHFITKNITDNHGKNKTTVHGYFSSIFHGGVDSLVDLMN
KSFPELGIKKTDCKEFSWIDTTIFYSGVVNFNTANFKKEILLDRSAGKKTAFSIKLDYVK
KPIPETAMVKILEKLYEEDVGVGMYVLYPYGGIMEEISESAIPFPHRAGIMYELWYTASW
EKQEDNEKHINWVRSVYNFTTPYVSQNPRLAYLNYRDLDLGKTNPESPNNYTQARIWGEK
YFGKNFNRLVKVKTKADPNNFFRNEQSIPPLPPHHH
THC1dNS2 Protein (Source Cannabis sativa)
(SEQ ID NO: 59)
MQLSLSVLSTVATALLSLTTAVDAKSHNPQENFLKCFSEYIPNNPANPKFIYTQHDQLYM
SVLNSTIQNLRFTSDTTPKPLVIVTPSNVSHIQASILCSKKVGLQIRTRSGGHDAEGMSY
ISQVPFVVVDLRNMHSIKIDVHSQTAWVEAGATLGEVYYWINEKNENFSFPGGYCPTVGV
GGHFSGGGYGALMRNYGLAADNIIDAHLVNVDGKVLDRKSMGEDLFWAIRGGGGENFGII
AAWKIKLVAVPSKSTIFSVKKNMEIHGLVKLFNKWQNIAYKYDKDLVLMTHFITKNITDN
HGKNKTTVHGYFSSIFHGGVDSLVDLMNKSFPELGIKKTDCKEFSWIDTTIFYSGVVNFN
TANFKKEILLDRSAGKKTAFSIKLDYVKKPIPETAMVKILEKLYEEDVGVGMYVLYPYGG
IMEEISESAIPFPHRAGIMYELWYTASWEKQEDNEKHINWVRSVYNFTTPYVSQNPRLAY
LNYRDLDLGKTNPESPNNYTQARIWGEKYFGKNFNRLVKVKTKADPNNFFRNEQSIPPLP
PHHH
THC1dNV1 Protein (Source Cannabis sativa)
(SEQ ID NO: 60)
MQLSLSVLSTVATALLSLTTAVDAKSHNIKLSKLSNEETLDASTFQEYTSSLANKYMNLF
NAAHGNPTSFGLQHVLSNQEAEVPFVTPQKGGNPQENFLKCFSEYIPNNPANPKFIYTQH
DQLYMSVLNSTIQNLRFTSDTTPKPLVIVTPSNVSHIQASILCSKKVGLQIRTRSGGHDA
EGMSYISQVPFVVVDLRNMHSIKIDVHSQTAWVEAGATLGEVYYWINEKNENFSFPGGYC
PTVGVGGHFSGGGYGALMRNYGLAADNIIDAHLVNVDGKVLDRKSMGEDLFWAIRGGGGE
NFGIIAAWKIKLVAVPSKSTIFSVKKNMEIHGLVKLFNKWQNIAYKYDKDLVLMTHFITK
NITDNHGKNKTTVHGYFSSIFHGGVDSLVDLMNKSFPELGIKKTDCKEFSWIDTTIFYSG
VVNFNTANFKKEILLDRSAGKKTAFSIKLDYVKKPIPETAMVKILEKLYEEDVGVGMYVL
YPYGGIMEEISESAIPFPHRAGIMYELWYTASWEKQEDNEKHINWVRSVYNFTTPYVSQN
PRLAYLNYRDLDLGKTNPESPNNYTQARIWGEKYFGKNFNRLVKVKTKADPNNFFRNEQS
IPPLPPHHH
THC1dNP1 Protein (Source Cannabis sativa)
(SEQ ID NO: 61)
MSNPQENFLKCFSEYIPNNPANPKFIYTQHDQLYMSVLNSTIQNLRFTSDTTPKPLVIVT
PSNVSHIQASILCSKKVGLQIRTRSGGHDAEGMSYISQVPFVVVDLRNMHSIKIDVHSQT
AWVEAGATLGEVYYWINEKNENFSFPGGYCPTVGVGGHFSGGGYGALMRNYGLAADNIID
AHLVNVDGKVLDRKSMGEDLFWAIRGGGGENFGIIAAWKIKLVAVPSKSTIFSVKKNMEI
HGLVKLFNKWQNIAYKYDKDLVLMTHFITKNITDNHGKNKTTVHGYFSSIFHGGVDSLVD
LMNKSFPELGIKKTDCKEFSWIDTTIFYSGVVNFNTANFKKEILLDRSAGKKTAFSIKLD
YVKKPIPETAMVKILEKLYEEDVGVGMYVLYPYGGIMEEISESAIPFPHRAGIMYELWYT
ASWEKQEDNEKHINWVRSVYNFTTPYVSQNPRLAYLNYRDLDLGKTNPESPNNYTQARIW
GEKYFGKNFNRLVKVKTKADPNNFFRNEQSIPPLPPHHHGRRAKL
CBC1 Protein (Source Cannabis sativa)
(SEQ ID NO: 62)
MNCSTFSFWFVCKIIFFFLSFNIQISIANPQENFLKCFSEYIPNNPANPKFIYTQHDQLY
MSVLNSTIQNLRFTSDTTPKPLVIVTPSNVSHIQASILCSKKVGLQIRTRSGGHDAEGLS
YISQVPFAIVDLRNMHTVKVDIHSQTAWVEAGATLGEVYYWINEMNENFSFPGGYCPTVG
VGGHFSGGGYGALMRNYGLAADNIIDAHLVNVDGKVLDRKSMGEDLFWAIRGGGGENFGI
IAACKIKLVVVPSKATIFSVKKNMEIHGLVKLFNKWQNIAYKYDKDLMLTTHFRTRNITD
NHGKNKTTVHGYFSSIFLGGVDSLVDLMNKSFPELGIKKTDCKELSWIDTTIFYSGVVNY
NTANFKKEILLDRSAGKKTAFSIKLDYVKKLIPETAMVKILEKLYEEEVGVGMYVLYPYG
GIMDEISESAIPFPHRAGIIVIYELWYTATWEKQEDNEKHINWVRSVYNFTTPYVSQNPR
LAYLNYRDLDLGKTNPESPNNYTQARIWGEKYFGKNFNRLVKVKTKADPNNFFRNEQSIP
PLPPRHH

### Promoters for Use in Candida

Multiple promoters can be used including synthetic ones. The following are some examples.

PEX11 Promoter Cv
(SEQ ID NO: 63)
GAGGATGAAGAAGACGAAGACGAATTGGATGAAGATGAAGCGTATGAGTA
TTATGAGTACTGTCGGACGTTGGAAGGTGGCAGAGTTAAGCCCGAGAAAG
CAAGGAAGGAGTGGGAGATGATGAGTGATGCGGCCAAGAGGATGTGAAGG
CTGCGTATCTGTTTTTGATAGCTGGTGGTAGCCGAATAGAGGAAGGCAAG
CTTGTTCATATTGGATGATGATGGTAGATGGTGGCTGCCAAAGTGGTTGT
AAATAGAAAAAAGTGGGTTTGGGTCTGTTGATAGTTAGTGGTGGCGGCTG
TCTGTGATTACGTCAGCAAGTAGCACCTCGGCAGTTAAAACAGCAGCAAC
AGAAAAAAAATGTGTGAAAGTTTGATTCCCCCACAGTCTACCACACCCAG
AGTTCCATTTATCCATAATATCACAAGCAATAGAAAAATAAAAAATTATC
AACAAATCACAACGAAAAGATTCTGCAAAATTATTTTCACTTCTTCTTTT
GACTTCCTCTTCTTCTTGTTAGGTTCTTTCCATATTTTCCCCTTAAACCC
ATACACAACGCAGCCAT
PoX4 Promoter CV
(SEQ ID NO: 64)
GAGCTCCAATTGTAATATTTCGGGAGAAATATCGTTGGGGTAAAACAACA
GAGAGAGAGAGGGAGAGATGGTTCTGGTAGAATTATAATCTGGTTGTTGC
AAATGCTACTGATCGACTCTGGCAATGTCTGTAGCTCGCTAGTTGTATGC
AACTTAGGTGTTATGCATACACACGGTTATTCGGTTGAATTGTGGAGTAA
AAATTGTCTGAGTTGTGTCTTAGCTACTGGCTGGCCCCCCGCGAAAGATA
ATCAAAATTACACTTGTGAATTTTTGCACACACACCGATTAACATTTCCC
TTTTTTGTCCACCGATACACGCTTGCCTCTTCTTATTTTCTCTGTGCTTC
CCCCTCCTGTGACTTTTTCCACCATTGATATAAAATCAACTCCATTTCCC
TAAAATCTCCCCAGATTCTAAAAACAACTTCTTCTCTTCTGCTTTTCCTT
ATTTTTGTTATATTTATTTACCATCCCTTATTTTGAATAGTTATTCCCCA
CTAACATTGTTCAAATCTTCACGACATAA
POX2 Promoter CV
(SEQ ID NO: 65)
GTGCCGACGGAGTAACAAAAATGTTACAGTGCGCACTACTTATCCCGCTA
AGGTGATAAACTGCAAAAACACACCGTCAAGGAGAAGTCGACGTTTTGCC
GCCACTTGTGAAGGGAAGAAGAGTCGTTGAGTTGATGTAATTAAGCTGGC
ACGTAGATACCAGAAGGTTCTAGAGTAGAGCTTGGGTGGTGTTTGGCCCT
GTTTGGACCACGGATAGAGATGGAGAATCCCTTGGTTAGAGCGGAGAGGA
AAAAATTGAAACTTTGCATATCCCACTTCATTATCCTTGATGTAACCGTT
TTATGGGGTAATTAAAGTGTGGAAAAATAATCAGGGAGACATATTCCCGA
TCAATTGGGTGGTGGTCGCTCAATTTCTGTGAGTAGTAGGCTCAGTGGTG
TGTATTGGGATTGGTAGTAGTCTGTATAAGCAGTGTTATATAACCCATTG
CTTGTTGATTCCTATTTTGCTGGCAAAAGTGACAACTGTAGTTGTGAGAT
AATCCTCGGTTATTACGCCTGGGGGGGCAGACAGCCAAAGTTGTGCCCGT
GCGACAATGGCATCAGAAGAAACAGAAAAAAAAAACACAGGCATTTTTAT
CCACATGCACACTACCCCCACTATTCCTGTCTGCAGTGTGCTTGTGTGTG
GCCCCCCGCAGAATCAACAGGGCAAACTCTGGAGCCTGAATCTTTATATA
AACTTCAGGCATTGGCCCCCCTTTTCACAATTCTTCACATCCACCATTTT
TTTTCTTCTTTCCTACCATATTAGTTTTTTTTTATTCTTTTCCTACCTAT
CTGATTATTATCAAACATCTGGTCATCCTCAAAAGAAAGAAAGAAACTAT
AACAATCAATC
FOX3 Promoter CV
(SEQ ID NO: 66)
TGGATTATGTAATCACAGGCTGTTTCTCCATTCCTGCATGTAGGCTGGCC
CGCGGTATCAACCATCGTCCCGCTTCTTCTGGTTTTTTTTTTTTTTCCGC
TATGATATTTTTGATCTCTTGGGGGATTTGGTGGGTCTGCCCCCCCCGCT
ACTACAAGCTCAAACACCCGAAACCTTACAACACACACACACACATCCCG
CTTAGTTGCGGGTTGAAGAACGTGTATTCCCGTAGGGTTAATGGTGTGTC
CCCCCCTAGTCACCCGCTTCTGCCATTCTGGGTTTGCCTTCAAAGCTGGC
ATAAATGACGAAAAAAAAGCACAGCATCCTGCACACAACCCTGCTCAGTG
TGACAGGTGGTGGTGTAATAGAAAACCTCGGCTTAAAACCTCTGGTCAGA
GCATCAACTGCAATCTTGTCTTTTTCTGCTGCCCTCACATTCTCCCCACA
CATTCCCACCCTCAAGATTTCACAGGCAAAAACTGCAAATATATATAAAT
CTACAACCAATTCTTTCCCCAGATGGAAAATCTAATTTTTGTTCACCCTT
TTTCTTTCTCCTGCTATCACTGCTACTGCACATATTCAACACCACAACC
TEF1 promoter CV
(SEQ ID NO: 67)
GACTGGGAGAACAGACCAAGGATGCCCATACACCATTAATAACAAGCACA
CCTTGATAAATCTCTAGTGTTGACAAATTGGTGATTTGAACATGACTGTA
GAGAGAGAGACAAGTAACCACTGATGGATTGGTGGTGGCAAATAACCACT
TTAAATAAAGACCAACTCACACACAAAGACAGCAGGTCGTGTTCCTATTT
CCAATTTTCACAAGGAGAAGAATAAAAATTTTTCAATGAGATTAACTAAA
GAAACAGACAGGCAGCCCACAAGAAGAAGAAGAAGAAGAGGAAGAGGAAG
AAGAAGAAGAAGAAGAAGAAGAAGAGAAAAAAAATTTTTCCCTCTGCGTT
GCGTTGGGCTTGGGTTGCCAGCACCCACCATATATAACTCTCATCAAATA
CCCAGTAGAGAAAAATTTTTCCTCCCTCTTTTTCTTTCTTTCTTCTCCTT
CTTTTGCTACTCTTTCTGTTTTTCATCAAAAAGATATATATAATCAATCA
TG
TDH3 Promoter CV
(SEQ ID NO: 68)
ACCAAACGTTACTTTTTTTTTGCAATCGGATGGTATGGGTCTGGGGTTCA
CCTGTTTTGTAAAGCTACAGAAGGTGGCATATTTCTCTGATCAGGTGTTT
TTTTTTTCGGCTGCTGCTGCTCGTGGTGGTGTAGTGGTAGTGGTGTGTGT
GTGTGTGTGTGCGTGCGTGTGGAAGGACGCTTTTTGCTCTCTGACTCCTC
CCAATCAGAAGTTGCTATAGTGGTGAAACAACAATGGATGATAATGCCCC
GGGCGGTGCGTGTCCGACACAAACCACTACATTTTTTAGCTGGGAGCCTA
CTGCCACTACGACCCACCCACCCATGGTCAACAAAAAAATTCTGACAAAT
TATAAAATAACCCTTGAATTCCCCCTTGGAAAAATTTTTGGTATTTCTCT
CTCTCTTTTCCTTTCCCTCTTCTTTTTCTCTCCATCAATCAATTGACGTT
CAGTAACTCAATTAATTACATCACATCCCTCAATTAAAGAATTTAAACAA
TG

### Promoters for use in Yarrowia

Multiple promoters can be used including synthetic ones. The following are some examples.

POX2 Promoter Yl
(SEQ ID NO: 69)
ACGATTCCGCCAAGTGAGACTGGCGATCGGGAGAAGGGTTGGTGGTCATG
GGGGATAGAATTTGTACAAGTGGAAAAACCACTACGAGTAGCGGATTTGA
TACCACAAGTAGCAGAGATATACAGCAATGGTGGGAGTGCAAGTATCGGA
ATGTACTGTACCTCCTGTACTCGTACTCGTACGGCACTCGTAGAAACGGG
GCAATACGGGGGAGAAGCGATCGCCCGTCTGTTCAATCGCCACAAGTCCG
AGTAATGCTTGAGTATCGAAGTCTTGTACCTCCCTGTCAATCATGGCACC
ACTGGTCTTGACTTGTCTATTCATACTGGACAAGCGCCAGAGTTAAGCTT
GTAGCGAATTTCGCCCTCGGACATCACCCCATACGACGGACACACATGCC
CGACAAACAGCCTCTCTTATTGTAGCTGAAAGTATATTGAATGTGAACGT
GTACAATATCAGGTACCAGCGGGAGGTTACGGCCAAGGTGATACCGGAAT
AACCCTGGCTTGGAGATGGTCGGTCCATTGTACTGAAGTGTCCGTGTCGT
TTCCGTCACTGCCCCAATTGGACATGTTTGTTTTTCCGATCTTTCGGGCG
CCCTCTCCTTGTCTCCTTGTCTGTCTCCTGGACTGTTGCTACCCCATTTC
TTTGGCCTCCATTGGTTCCTCCCCGTCTTTCACGTCGTCTATGGTTGCAT
GGTTTCCCTTATACTTTTCCCCACAGTCACATGTTATGGAGGGGTCTAGA
TGGAGGCCTAATTTTGACGTGCAAGGGGCGAATTGGGGCGAGAAACACGT
CGTGGACATGGTGCAAGGCCCGCAGGGTTGATTCGACGCTTTTCCGCGAA
AAAAACAAGTCCAAATACCCCCGTTTATTCTCCCTCGGCTCTCGGTATTT
CACATGAAAACTATAACCTAGACTACACGGGCAACCTTAACCCCAGAGTA
TACTTATATACCAAAGGGATGGGTCCTCAAAAATCACACAAGCAACGACG
CCATGA
LIP2 promoter Yl
(SEQ ID NO: 70)
AAACTTCTCCGAGTCTGTGCCTTCAGGTGGGCATAGTTGATGGGTGTTTT
GAAGTTAATAGTGGGGAAGAACTATGGCAAACAAGCAGATGCAGGCACCT
TGTAACTGCAGACCGGTTCTTGTCTACCGACTCCGCTGCACCTGTGCCGC
GGTACATGTCGTCACAGGCTGCGGGGTTCGGAGGCCCCCTTGCAACCTCC
TTTGATAGTTGCTATGGCCCCAAAGAGTTATACGAGATAGACCCACAGAT
CTACTTGACTGTTGTCACAGAACCTGCTAGGTTTGCTTATTGTACCCGCT
TTGTAGCTACTGTACAACGACAACGTCAAAAATTGAGACGCGAACAAACT
CCAGATGCAGAACCCAAACCTCTCTCTCAGAGTTTCGAGTGCTTCTACCT
CACAGTAAAGTGGAGGTGGACCTGCAAGGGAATTCAGTCACAAGGCCCCG
AATGTCTCCGAAACTCCAATCGGACCGTTTAAACAGACTAATATCACGTC
ATTGATTGATATTAGCATCCGGCAAGAGCCGCAAGGTTATCTCCTCACCA
ATGAGCCTGTTGTACGGCTCATTCCGCATCTGCGGCTGATTCAGTTTCGA
GTGGGGATGGTAGACTTCATTGCAGCATTCCTAACCTTCTACTTGGTCCG
TGGAGATGTCATGGACATCGATTTTGGGCTGAGAAGCCTTTTGACGATGT
TGATATCACTGACCGCTAATTTACTCTGGCAGTTTCTCCGGCTCTCGAGG
CATCGTCGATCACCAAACACTATCTGCTAGTCTAAATGTCCGACACGACA
GCTTTTGATCGCCGTGAACGGCGCAGACCTCATGCACCATGCACCAGGGC
CAAATCAATTACGGGTCGCTTAGCGTTGCAGTCGGGGCATTATGGTGGAA
GTTCCGATACGGCACAGACACATTCCATAGTGGGGGGATTGGATTATAAA
AGGGCCATAGAAAGCCCTCAATTGATACCCAAGTACCAGCTCTCCTCACT
ATGA
ICL1 Promoter Yk
(SEQ ID NO: 71)
GACCCCCTCCTTTTGCCAGTATATCCACCGCAACACCCACCATGAGCGAC
ATCTGATACCGTGCCGCGACCACTACCCCAAATAAGCTCCAACTAATATG
CCGAGGCAGGTGGGAAACTATGCACTCCAGACGACGCTGTAGAAGCACAT
GGAAGGTGCGGAGGCGGTGGCAACGAGGGGCATGAGCCATCAACGAGTAA
CCACAGACAAGGCAAGGGGGGAAACGCGACCGGAATCTCTCGCGGTCACG
TGACCCGCCCGGGTTCCACTCGTCCATGTTGTGTCTCTGGTGTCTTCGGC
CGACTCGCATTGGTTAAACTTCCACCACCGCAATCACGTCCCACTGGCCA
AACTTTTTCTGCTTTCTCTGACTTTTTCTGGCCAAAAGGCAACGTCGGAA
AGGGTCGGGAGGATTCGGAACCGACGAAAATCGGCCGGCTCCAGCGGGGG
TAGTTCGGCAGTCCTGGTGGGAGCTCTAGGGGAGCTGTGGTCTGTGTAGG
GCGCGGGTCCGGGTTTGTTGGGTGTCAAATCACGTGTTTTTGCCCCCCCG
CTGAGCCGGACTCCGACAACCGTGTCTCCAACGGCCTGACTAAGCTGCTC
CCAGCACTCTGCCGTAGCGTTGGTCTGTCCTGTCGCACTCTGTTCAAAGA
CAGAAGAAAGAAAAAGCTAACCTCCACGTCAGAGACAATGGTAGAAGGCT
TGTTCCTTGCAACCGAGGAGAGTGAGTGTTCTCGGCACGAGCATCATGGG
CGATCTGGAGGGTATTTTTGAGGGGAAAAAACGGGATCAGGACAAACAGA
GGCCACAGACCGGGAATCTGGGCCCCAAAACGGCCTTTTCCCGTCGCAAA
ACCGGTCTACATACACCCCTTCGGCCCGCCACAGGCCGGTGTGAAAAACC
CTAAAGCTTGCTTCAAACCAGACGGACGCACAGCAAGACACATCATGAAG
AGTCACCTGCAGTATATATAGATCTGGGGATTCCCAGTAGACTGACCAAG
CATACAAAAGTGAGTATCCAACAGCGACACGTGAGATGGCAGAGACACAG
AGACGTGTCTACATGGTTGGACAAGTCTCCACATTCGCCAGAGACGTATC
CACATACAAACACAATCTCACAGCTGATCTGCTCCTGTGACAGCACAGTA
CATGTTAGTGGATGAGGTGTTGTGTAGTGGGTTAAATGGGTGGACTGATT
CAGTGGCATCGGTGGCGACACCCTCTACTCTTCATGTCGTCACCTACCGT
TCGGAATCCCAATTATCTGATGAACTAAACGATTTCTGGCCAAAACACAA
TTTTGCCAAAGAAGTCGTTCTCACCAATGCAAGTGTCACATCAAACATCT
GTCCCGTACTAACCCAG
POT1 Promoter Yl
(SEQ ID NO: 72)
CCACAATACCCCACAGTGTGCATATCAAACCTACCGGTTGTTGCTCTCTC
CAGCCTTACTAAGAAGGAGGCGACGTGGCAGTGGCTCGCGGGAGGATCGG
CGGGAAACTCCGGGATATCCGTCGAGAGTTTACACGTGAATGGGCAGCGC
AATCCGTTGACGACGATACGACTGGCAAAGTAGCGACGATACCTGCCAGA
CAGGTGACATGTGCAGGCCGCACTAACAAGGAAACGGGCGCTGGGGGGGG
CGGGCTTCTAGACTTTGCCCTTGAACAGGAATCTAGTGGGGGCTTGTCTT
TCCGCCAATGGGGGAGCGCCTGTTGAGCGACCGTGCATGCTGGAACGCCA
AGTGTATGTACAGCTGGTGTTCTCGCAGCGGTATGTGACGGGACTTACAT
CTCTCGTTTTTTCATGACCACGTTTTCACAGGCTCGGAGGTACGTTAAAG
TTTTGAAGGCTGCATCTGAACCGAGGTATGGGGGAGTTTGAGGAGCAACA
GTGTTGGGGCTGAGGGGGCCAAGATCGGGGCAAGCAGAGGTCTTAGATCA
ATTGTGGGAATCCCAAAGGGCTCGTTATCACCTTTTTCCACCCAATTCGG
GTCCCAATTGATCCACTACTGGCTTGCCCAAGTTACCCCAGAAATGCCGC
CCCGGATTTCTCCAAAAACCTAATAAGCTTCATGGAACTTGGTGGAAGTG
ACTTTCTACAGAGTGGAGAGAACCGTGGACACGTGGCAATGGCGCTGACC
GTGTCCCCGAGCCGAATCGACGTGAGGGGAGAACGGAGTATCTGCGGTCA
TGTGACCTTCCAGAGCGGCGTCGCCAGTGTGCACGCGGTGACCCCCAGTT
TGGTTCTCTGTCACACGCATACTACCTCGGCTCTCCACATGCTGAACTTT
ATCTTTCGTGGGGATCATACCGAAAGTTGCAACTACCAGGTGTATATAAA
GCCTGGTAGACTCCCCCCACTTTGGACCTCATCCAACCAAGACACACAAA
AATG

Terminators to Use in Candida viswanathii

Multiple terminators can be used. The following are some examples.

POX4 Terminator Cv
(SEQ ID NO: 73)
GAGTGACTCTTTTGATAAGAGTCGCAAATTTGATTTCATAAGTATATATT
CATTATGTAAAGTAGTAAATGGAAAATTCATTAAAAAAAAAGCAAATTTC
CGTTGTATGCATACTCCGAACACAAAACTAGCCCCGGAAAAACCCTTAGT
TGATAGTTGCGAATTTAGGTCGAC
PEX11 Terminator CV
(SEQ ID NO: 74)
AGCTCCAGGCTTGTTATGACTCTAGAGAGAAGTGTGTGTGTTTGCGTTTG
TTTTACTATACATTCAACATGTTCTTTTTCTTTTTTGATATTTATTCCAA
CTATAATTATACACAGATTCGTATATACTTTACTTTACCCTCTTTCGTAG
TTTTTTAATTTGATGATTTTTGAGTTTCATATCCAAGGTCAAAACCCGAC

Terminators to use in Yarrowia lipolytica

Multiple terminators can be used. The following are some examples.

XPR2 Terminato Yl
(SEQ ID NO: 75)
TAGGCAATTAACAGATAGTTTGCCGGTGATAATTCTCTTAACCTCCCACA
CTCCTTTGACATAACGATTTATGTAACGAAACTGAAATTTGACCAGATAT
TGTTGTAAATAGAAAATCTGGCTTGTAGGTGGCAAAATGCGGCGTCTTTG
TTCATCAATTCCCTCTGTGACTACTCGTCATCCCTTTATGTTCGACTGTC
GTATTTCTTATTTTCCATACATATGCAAGTGAGATGCCCGTGTCCGAATT
C
TDH3 terminator YL
(SEQ ID NO: 76)
CTTCCGAGTAGCTATCCGAAGATCAAGAGCGAAGCAAGTTGTAAGTCCAG
GACATGTTTCCCGCCCACGCGAGTGATTTATAACACCTCTCTTTTTTGAC
ACCCGCTCGCCTTGAAATTCATGTCACATAAATTATAGTCAACGACGTTT
GAATAACTTGTCTTGTAGTTCGATGATGATCATATGATTACATTAATAGT
AATTACTGTATTTGATATATATACTAATTACAATAGTACATATTAGAACA
TACAATAGTTAGTGCCGTGAAGTGGCTTAAAATACCGCGAGTCGATTACG
TAATATTA

### Markers for Use in Yarrowia

LEU2 Marker Yl
(SEQ ID NO: 77)
AACGTACCACTGTCCTCCACTACAAACACACCCAATCTGCTTCTTCTAGTCAAGGTT
GCTACACCGGTAAATTATAAATCATCATTTCATTAGCAGGGCTGGGCCCTTTTTATA
GAGTCTTATACACTAGCGGACCCTGCCGGTAGACCAACCCGCAGGCGCGTCAGTTTG
CTCCTTCCATCAATGCGTCGTAGAAACGACTTACTCCTTCTTGAGCAGCTCCTTGACC
TTGTTGGCAACAAAGTCTCCGACCTCGGAGGTGGAGGAGGAGCCTCCGATATCGGC
GGTAGTGATACCAGCCTCGACGGACTCCTTGACGGCAGCCTCAACAGCGTCACCGG
CGGGCTTCATGTTAAGAGAGAACTTGAGCATCATGGCGGCAGACAGAATGGTGGCA
ATGGGGTTGACCTTCTGCTTGCCGAGATCGGGGGCAGATCCGTGACAGGGCTCGTAC
AGACCGAACGCCTCGTTGGTGTCGGGCAGAGAAGCCAGAGAGGCGGAGGGCAGCA
GACCCAGAGAACCGGGGATGACGGAGGCCTCGTCGGAGATGATATCGCCAAACATG
TTGGTGGTGATGATGATACCATTCATCTTGGAGGGCTGCTTGATGAGGATCATGGCG
GCCGAGTCGATCAGCTGGTGGTTGAGCTCCAGCTGGGGGAATTCGTCCTTGAGGACT
CGGGTGACGGTCTTTCGCCAAAGTCGAGAGGAGGCCAGCACGTTGGCCTTGTCAAG
GGACCACACGGGAAGAGGGGGGTTGTGCTGAAGGGCCAGGAAGGCGGCCATTCGG
GCAATTCGCTCAACCTCAGGAACGGAGTAAGTCTCAGTGTCGGAAGCGACGCCAGA
TCCGTCATCCTCCTTTCGCTCTCCAAAGTAGATACCTCCGACGAGCTCTCGGACAAT
GATGAAGTCGGTGCCCTCAACGTTTCGGATGGGGGAGAGATCGGCGAGCTTGGGCG
ACAGCAGCTGGCAGGGTCGCAGGTTGGCGTACAGGTTCAGGTCCTTTCGCAGCTTGA
GAAGACCCTGCTCGGGTCGCACGTCGGTTCGTCCGTCGGGAGTGGTCCATACGGTGT
TGGCAGCGCCTCCGACAGCACCGAGCATAATAGAGTCAGCCTTTCGGCAGATGTCG
AGAGTAGCGTCGGTGATGGGCTCGCCCTCCTTCTCAATGGCAGCTCCTCCAATGAGT
CGGTCCTCAAACACAAACTCGGTGCCGGAGGCCTCAGCAACAGACTTGAGCACCTT
GACGGCCTCGGCAATCACCTCGGGGCCACAGAAGTCGCCGCCGAGAAGAACAATCT
TCTTGGAGTCAGTCTTGGTCTTCTTAGTTTCGGGTTCCATTGTGGATGTGTGTGGTTG
TATGTGTGATGTGGTGTGTGGAGTGAAAATCTGTGGCTGGCAAACGCTCTTGTATAT
ATACGCACTTTTGCCCGTGCTATGTGGAAGACTAAACCTCCGAAGATTGTGACTCAG
GTAGTGCGGTATCGGCTAGGGACCCAAACCTTGTCGATGCCGATAGCGCTATCGAAC
GTACCCCAGCCGGCCGGGAGTATGTCGGAGGGGACATACGAGATCGTCAAGGGTTT
GTGGCCAACTGGTAAATAAATGATGACTCAGGCGACGACGGAATTCGACAGCAACT
ACTCCTTTCACCAACCATGTGCATTTTAGCTCGAATAACATTCACAGGCTTGGTGATC
TACATCCATGGTGTCTGGCCGATTACCGTGGTGTTTTGGCAGTAACGAGAATATTGA
GTGAACTCTTCCCATCACCAATAAAGACTCATACTACAATCACGAGCGCTTCAGCTG
CCACTATAGTGTTGGTGACACAATACCCCTCGATGCTGGGCATTACTGTAGCAAGAG
ATATTATTTCATGGCGCATTTTCCAGTCTACCTGACTTTTTAGTGTGATTTCTTCTCCA
CATTTTATGCTCAGTGTGAAAAGTTGGAGTGCACACTTAATTATCGCCGGTTTTCGG
AAAGTACTATGTGCTCAAGGTTGCACCCCACGTTACGTATGCAGCACATTGAGCAGC
CTTTGGACCGTGGAGATAACGGTGTGGAGATAGCAACGGGTAGTCTTCGTATTAATT
CAATGCATTGTTAGTTTTATATGATATGGTGTCGA
URA3 Marker Yl
(SEQ ID NO: 78)
TTTCTAATTTGGACCGATAGCCGTATAGTCCAGTCTATCTATAAGTTCAACTAACTCG
TAACTATTACCATAACATATACTTCACTGCCCCAGATAAGGTTCCGATAAAAAGTTG
TGCAGACTAAATTTATTTCAGTCTCCTCTTCACCACCAAAATGCCCTCCTACGAAGC
GCGAGCTAACGTCCACAAGTCCGCCTTTGCCGCCCGAGTGCTCAAGCTCGTGGCAGC
CAAGAAAACCAACCTGTGTGCTTCTCTGGATGTTACCACCACCAAGGAGCTCATTGA
GCTTGCCGATAAGGTCGGACCTTATGTGTGCATGATCAAGACCCATATCGACATCAT
TGACGACTTCACCTACGCCGGAACTGTGCTCCCCCTCAAGGAACTTGCTCTTAAGCA
CGGTTTCTTCCTGTTCGAGGACAGAAAGTTCGCAGATATTGGCAACACTGTCAAGCA
CCAGTACAAGAACGGTGTCTACCGAATCGCCGAGTGGTCCGATATCACCAACGCCC
ACGGTGTACCCGGAACCGGAATCATTGCTGGCCTGCGAGCTGGTGCCGAGGAAACT
GTCTCTGAACAGAAGAAGGAGGATGTCTCTGACTACGAGAACTCCCAGTACAAGGA
GTTCCTGGTCCCCTCTCCCAACGAGAAGCTGGCCAGAGGTCTGCTCATGCTGGCCGA
GCTGTCTTGCAAGGGCTCTCTGGCCACTGGCGAGTACTCCAAGCAGACCATTGAGCT
TGCCCGATCCGACCCCGAGTTTGTGGTTGGCTTCATTGCCCAGAACCGACCTAAGGG
CGACTCTGAGGACTGGCTTATTCTGACCCCCGGGGTGGGTCTTGACGACAAGGGAG
ATGCTCTCGGACAGCAGTACCGAACTGTTGAGGATGTCATGTCTACCGGAACGGATA
TCATAATTGTCGGCCGAGGTCTGTACGGCCAGAACCGAGATCCTATTGAGGAGGCC
AAGCGATACCAGAAGGCTGGCTGGGAGGCTTACCAGAAGATTAACTGTTAGAGGTT
AGACTATGGATATGTCATTTAACTGTGTATATAGAGAGCGTGCAAGTATGGAGCGCT
TGTTCAGCTTGTATGATGGTCAGACGACCTGTCTGATCGAGTATGTATGATACTGCA
CAACCTG

### Polynucleotides and Polypeptides

A nucleic acid (e.g., also referred to herein as nucleic acid reagent, target nucleic acid, target nucleotide sequence, nucleic acid sequence of interest or nucleic acid region of interest) can be from any source or composition, such as DNA, cDNA, gDNA (genomic DNA), RNA, siRNA (short inhibitory RNA), RNAi, tRNA or mRNA, for example, and can be in any form (e.g., linear, circular, supercoiled, single-stranded, double-stranded, and the like). A nucleic acid can also comprise DNA or RNA analogs (e.g., containing base analogs, sugar analogs and/or a non-native backbone and the like). It is understood that the term “nucleic acid” does not refer to or infer a specific length of the polynucleotide chain, thus polynucleotides and oligonucleotides are also included in the definition. Deoxyribonucleotides include deoxyadenosine, deoxycytidine, deoxyguanosine and deoxythymidine. For RNA, the uracil base is uridine.

A nucleic acid sometimes is a plasmid, phage, autonomously replicating sequence (ARS), centromere, artificial chromosome, yeast artificial chromosome (e.g., YAC) or other nucleic acid able to replicate or be replicated in a host cell. In certain embodiments a nucleic acid can be from a library or can be obtained from enzymatically digested, sheared, or sonicated genomic DNA (e.g., fragmented) from an organism of interest. In some embodiments, nucleic acid subjected to fragmentation or cleavage may have a nominal, average or mean length of about 5 to about 10,000 base pairs, about 100 to about 1,000 base pairs, about 100 to about 500 base pairs, or about 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000 or 10000 base pairs. Fragments can be generated by any suitable method in the art, and the average, mean or nominal length of nucleic acid fragments can be controlled by selecting an appropriate fragment-generating procedure by the person of ordinary skill. In some embodiments, the fragmented DNA can be size selected to obtain nucleic acid fragments of a particular size range.

Nucleic acid can be fragmented by various methods known to the person of ordinary skill, which include without limitation, physical, chemical and enzymic processes. Examples of such processes are described in U.S. Patent Application Publication No. 20050112590 (published on May 26, 2005, entitled “Fragmentation-based methods and systems for sequence variation detection and discovery,” naming Van Den Boom et al.). Certain processes can be selected by the person of ordinary skill to generate non-specifically cleaved fragments or specifically cleaved fragments. Examples of processes that can generate non-specifically cleaved fragment sample nucleic acid include, without limitation, contacting sample nucleic acid with apparatus that expose nucleic acid to shearing force (e.g., passing nucleic acid through a syringe needle; use of a French press); exposing sample nucleic acid to irradiation (e.g., gamma, x-ray, UV irradiation; fragment sizes can be controlled by irradiation intensity); boiling nucleic acid in water (e.g., yields about 500 base pair fragments) and exposing nucleic acid to an acid and base hydrolysis process.

Nucleic acid may be specifically cleaved by contacting the nucleic acid with one or more specific cleavage agents. The term “specific cleavage agent” as used herein refers to an agent, sometimes a chemical or an enzyme that can cleave a nucleic acid at one or more specific sites. Specific cleavage agents often will cleave specifically according to a particular nucleotide sequence at a particular site. Examples of enzymic specific cleavage agents include without limitation endonucleases (e.g., DNase (e.g., DNase I, II); RNase (e.g., RNase E, F, H, P); Cleavase™ enzyme; Taq DNA polymerase; E. coli DNA polymerase I and eukaryotic structure-specific endonucleases; murine FEN-1 endonucleases; type I, II or III restriction endonucleases such as Acc I, Afi III, Alu I, Alw44 I, Apa I, Asn I, Ava I, Ava II, BamH I, Ban II, Bcl I, Bgl I. Bgl II, Bln I, Bsa I, Bsm I, BsmBI, BssH II, BstE II, Cfo I, Cla I, Dde I, Dpn I, Dra I, EclX I, EcoR I, EcoR I, EcoR II, EcoR V, Hae II, Hae II, Hind III, Hind III, Hpa I, Hpa II, Kpn I, Ksp I, Mlu I, MluN I, Msp I, Nci I, Nco I, Nde I, Nde II, Nhe I, Not I, Nru I, Nsi I, Pst I, Pvu I, Pvu II, Rsa I, Sac I, Sap I, Sal I, Sau3A I, Sea I, ScrF I, Sfi I, Sma I, Spe I, Sph I, Ssp I, Stu I, Sty I, Swa I, Taq I, Xba I, Xho I); glycosylases (e.g., uracil-DNA glycolsylase (UDG), 3-methyladenine DNA glycosylase, 3-methyladenine DNA glycosylase II, pyrimidine hydrate-DNA glycosylase, FaPy-DNA glycosylase, thymine mismatch-DNA glycosylase, hypoxanthine-DNA glycosylase, 5-Hydroxymethyluracil DNA glycosylase (HmUDG), 5-Hydroxymethylcytosine DNA glycosylase, or 1,N6-etheno-adenine DNA glycosylase); exonucleases (e.g., exonuclease III); ribozymes, and DNAzymes. Sample nucleic acid may be treated with a chemical agent, or synthesized using modified nucleotides, and the modified nucleic acid may be cleaved. In non-limiting examples, sample nucleic acid may be treated with (i) alkylating agents such as methylnitrosourea that generate several alkylated bases, including N3-methyladenine and N3-methylguanine, which are recognized and cleaved by alkyl purine DNA-glycosylase; (ii) sodium bisulfite, which causes deamination of cytosine residues in DNA to form uracil residues that can be cleaved by uracil N-glycosylase; and (iii) a chemical agent that converts guanine to its oxidized form, 8-hydroxyguanine, which can be cleaved by formamidopyrimidine DNA N-glycosylase. Examples of chemical cleavage processes include without limitation alkylation, (e.g., alkylation of phosphorothioate-modified nucleic acid); cleavage of acid lability of P3′-N5′-phosphoroamidate-containing nucleic acid; and osmium tetroxide and piperidine treatment of nucleic acid.

As used herein, the term “complementary cleavage reactions” refers to cleavage reactions that are carried out on the same nucleic acid using different cleavage reagents or by altering the cleavage specificity of the same cleavage reagent such that alternate cleavage patterns of the same target or reference nucleic acid or protein are generated. In certain embodiments, nucleic acids of interest may be treated with one or more specific cleavage agents (e.g., 1, 2, 3, 4, 5, 6, 7, 8, 9, 10 or more specific cleavage agents) in one or more reaction vessels (e.g., nucleic acid of interest is treated with each specific cleavage agent in a separate vessel).

A nucleic acid suitable for use in the embodiments described herein sometimes is amplified by any amplification process known in the art (e.g., PCR, RT-PCR and the like). Nucleic acid amplification may be particularly beneficial when using organisms that are typically difficult to culture (e.g., slow growing, require specialized culture conditions and the like). The terms “amplify”, “amplification”, “amplification reaction”, or “amplifying” as used herein, refer to any in vitro processes for multiplying the copies of a target sequence of nucleic acid. Amplification sometimes refers to an “exponential” increase in target nucleic acid. However, “amplifying” as used herein can also refer to linear increases in the numbers of a select target sequence of nucleic acid, but is different than a one-time, single primer extension step. In some embodiments, a limited amplification reaction, also known as pre-amplification, can be performed. Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification may also limit inaccuracies associated with depleted reactants in standard PCR reactions.

In some embodiments, a nucleic acid reagent sometimes is stably integrated into the chromosome of the host organism, or a nucleic acid reagent can be a deletion of a portion of the host chromosome, in certain embodiments (e.g., genetically modified organisms, where alteration of the host genome confers the ability to selectively or preferentially maintain the desired organism carrying the genetic modification). Such nucleic acid reagents (e.g., nucleic acids or genetically modified organisms whose altered genome confers a selectable trait to the organism) can be selected for their ability to guide production of a desired protein or nucleic acid molecule. When desired, the nucleic acid reagent can be altered such that codons encode for (i) the same amino acid, using a different tRNA than that specified in the native sequence, or (ii) a different amino acid than is normal, including unconventional or unnatural amino acids (including detectably labeled amino acids). As described herein, the term “native sequence” refers to an unmodified nucleotide sequence as found in its natural setting (e.g., a nucleotide sequence as found in an organism).

A nucleic acid or nucleic acid reagent can comprise certain elements often selected according to the intended use of the nucleic acid. Any of the following elements can be included in or excluded from a nucleic acid reagent. A nucleic acid reagent, for example, may include one or more or all of the following nucleotide elements: one or more promoter elements, one or more 5′ untranslated regions (5′UTRs), one or more regions into which a target nucleotide sequence may be inserted (an “insertion element”), one or more target nucleotide sequences, one or more 3′ untranslated regions (3′UTRs), and one or more selection elements. A nucleic acid reagent can be provided with one or more of such elements and other elements may be inserted into the nucleic acid before the nucleic acid is introduced into the desired organism. In some embodiments, a provided nucleic acid reagent comprises a promoter, 5′UTR, optional 3′UTR, and insertion element(s) by which a target nucleotide sequence is inserted (i.e., cloned) into the nucleotide acid reagent. In certain embodiments, a provided nucleic acid reagent comprises a promoter, insertion element(s) and optional 3′UTR, and a 5′ UTR/target nucleotide sequence is inserted with an optional 3′UTR. The elements can be arranged in any order suitable for expression in the chosen expression system (e.g., expression in a chosen organism, or expression in a cell free system, for example), and in some embodiments a nucleic acid reagent comprises the following elements in the 5′ to 3′ direction: (1) promoter element, 5′UTR, and insertion element(s); (2) promoter element, 5′UTR, and target nucleotide sequence; (3) promoter element, 5′UTR, insertion element(s) and 3′UTR; and (4) promoter element, 5′UTR, target nucleotide sequence and 3′UTR.

A promoter element typically is required for DNA synthesis and/or RNA synthesis. A promoter element often comprises a region of DNA that can facilitate the transcription of a particular gene, by providing a start site for the synthesis of RNA corresponding to a gene. Promoters generally are located near the genes they regulate, are located upstream of the gene (e.g., 5′ of the gene), and are on the same strand of DNA as the sense strand of the gene, in some embodiments.

A promoter often interacts with an RNA polymerase. A polymerase is an enzyme that catalyzes synthesis of nucleic acids using a preexisting nucleic acid reagent. When the template is a DNA template, an RNA molecule is transcribed before protein is synthesized. Enzymes having polymerase activity suitable for use in the present methods include any polymerase that is active in the chosen system with the chosen template to synthesize protein. In some embodiments, a promoter (e.g., a heterologous promoter) also referred to herein as a promoter element, can be operably linked to a nucleotide sequence or an open reading frame (ORF). Transcription from the promoter element can catalyze the synthesis of an RNA corresponding to the nucleotide sequence or ORF sequence operably linked to the promoter, which in turn leads to synthesis of a desired peptide, polypeptide, or protein. The term “operably linked” as used herein with respect to promoters refers to a nucleic acid sequence (e.g., a coding sequence) present on the same nucleic acid molecule as a promoter element and whose expression is under the control of said promoter element.

Promoter elements sometimes exhibit responsiveness to regulatory control. Promoter elements also sometimes can be regulated by a selective agent. That is, transcription from promoter elements sometimes can be turned on, turned off, up-regulated or down-regulated, in response to a change in environmental, nutritional or internal conditions or signals (e.g., heat inducible promoters, light regulated promoters, feedback regulated promoters, hormone influenced promoters, tissue specific promoters, oxygen and pH influenced promoters, promoters that are responsive to selective agents (e.g., kanamycin) and the like, for example). Promoters influenced by environmental, nutritional, or internal signals frequently are influenced by a signal (direct or indirect) that binds at or near the promoter and increases or decreases expression of the target sequence under certain conditions.

Non-limiting examples of selective or regulatory agents that can influence transcription from a promoter element used in embodiments described herein include, without limitation, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like). In some embodiments, the regulatory or selective agent can be added to change the existing growth conditions to which the organism is subjected (e.g., growth in liquid culture, growth in a fermentor, growth on solid nutrient plates and the like for example).

In some embodiments, regulation of a promoter element can be used to alter (e.g., increase, add, decrease, or substantially eliminate) the activity of a peptide, polypeptide, or protein (e.g., enzyme activity for example). For example, a microorganism can be engineered by genetic modification to express a nucleic acid reagent that can add a novel activity (e.g., an activity not normally found in the host organism) or increase the expression of an existing activity by increasing transcription from a homologous or heterologous promoter operably linked to a nucleotide sequence of interest (e.g., homologous or heterologous nucleotide sequence of interest), in certain embodiments. In some embodiments, a microorganism can be engineered by genetic modification to express a nucleic acid reagent that can decrease expression of an activity by decreasing or substantially eliminating transcription from a homologous or heterologous promoter operably linked to a nucleotide sequence of interest, in certain embodiments.

In some embodiments the activity can be altered using recombinant DNA and genetic techniques known to the artisan. Methods for engineering microorganisms are further described herein. Tables herein provide non-limiting lists of yeast promoters that are up-regulated by oxygen, yeast promoters that are down-regulated by oxygen, yeast transcriptional repressors and their associated genes, DNA binding motifs as determined using the MEME sequence analysis software. Potential regulator binding motifs can be identified using the program MEME to search intergenic regions bound by regulators for overrepresented sequences. For each regulator, the sequences of intergenic regions bound with p-values less than 0.001 were extracted to use as input for motif discovery. The MEME software was run using the following settings: a motif width ranging from 6 to 18 bases, the “zoops” distribution model, a 6th order Markov background model, and a discovery limit of 20 motifs. The discovered sequence motifs were scored for significance by two criteria: an E-value calculated by MEME and a specificity score. The motif with the best score using each metric is shown for each regulator. All motifs presented are derived from datasets generated in rich growth conditions with the exception of a previously published dataset for epitope-tagged Gal4 grown in galactose.

In some embodiments, the altered activity can be found by screening the organism under conditions that select for the desired change in activity. For example, certain microorganisms can be adapted to increase or decrease an activity by selecting or screening the organism in question on a media containing substances that are poorly metabolized or even toxic. An increase in the ability of an organism to grow a substance that is normally poorly metabolized would result in an increase in the growth rate of that substance, for example. A decrease in the sensitivity to a toxic substance might be manifested by growth on higher concentrations of the toxic substance, for example. Genetic modifications that are identified in this manner sometimes are referred to as naturally occurring mutations or the organisms that carry them can sometimes be referred to as naturally occurring mutants. Modifications obtained in this manner are not limited to alterations in promoter sequences. That is, screening microorganisms by selective pressure, as described above, can yield genetic alterations that can occur in non-promoter sequences, and sometimes also can occur in sequences that are not in the nucleotide sequence of interest, but in a related nucleotide sequences (e.g., a gene involved in a different step of the same pathway, a transport gene, and the like). Naturally occurring mutants sometimes can be found by isolating naturally occurring variants from unique environments, in some embodiments.

In addition to the regulated promoter sequences, regulatory sequences, and coding polynucleotides provided herein, a nucleic acid reagent may include a polynucleotide sequence 70% or more identical to the foregoing (or to the complementary sequences). That is, a nucleotide sequence that is at least 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to a nucleotide sequence described herein can be utilized. The term “identical” as used herein refers to two or more nucleotide sequences having substantially the same nucleotide sequence when compared to each other. One test for determining whether two nucleotide sequences or amino acids sequences are substantially identical is to determine the percent of identical nucleotide sequences or amino acid sequences shared.

Calculations of sequence identity can be performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70% or more, 80% or more, 90% or more, or 100% of the length of the reference sequence. The nucleotides or amino acids at corresponding nucleotide or polypeptide positions, respectively, are then compared among the two sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the http address at world wide web uniform resource locator gcg.com), using either a Blossum 62 matrix or a PAM250 matrix, and a gap weight of 16, 14, 12, 10, 8, 6, or 4 and a length weight of 1, 2, 3, 4, 5, or 6. Percent identity between two nucleotide sequences can be determined using the GAP program in the GCG software package (available at http address at world wide web uniform resource locator gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Sequence identity can also be determined by hybridization assays conducted under stringent conditions. As used herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

As noted above, nucleic acid reagents may also comprise one or more 5′ UTRs, and one or more 3′UTR's. A 5′ UTR may comprise one or more elements endogenous to the nucleotide sequence from which it originates, and sometimes includes one or more exogenous elements. A 5′ UTR can originate from any suitable nucleic acid, such as genomic DNA, plasmid DNA, RNA, or mRNA, for example, from any suitable organism (e.g., virus, bacterium, yeast, fungi, plant, insect, or mammal). The artisan may select appropriate elements for the 5′ UTR based upon the chosen expression system (e.g., expression in a chosen organism, or expression in a cell free system, for example). A 5′ UTR sometimes comprises one or more of the following elements known to the artisan: enhancer sequences (e.g., transcriptional or translational), transcription initiation site, transcription factor binding site, translation regulation site, translation initiation site, translation factor binding site, accessory protein binding site, feedback regulation agent binding sites, Pribnow box, TATA box, −35 element, E-box (helix-loop-helix binding element), ribosome binding site, replicon, internal ribosome entry site (IRES), silencer element and the like. In some embodiments, a promoter element may be isolated such that all 5′ UTR elements necessary for proper conditional regulation are contained in the promoter element fragment, or within a functional subsequence of a promoter element fragment.

A 5 ′UTR in the nucleic acid reagent can comprise a translational enhancer nucleotide sequence. A translational enhancer nucleotide sequence often is located between the promoter and the target nucleotide sequence in a nucleic acid reagent. A translational enhancer sequence often binds to a ribosome, sometimes is an 18S rRNA-binding ribonucleotide sequence (i.e., a 40S ribosome binding sequence) and sometimes is an internal ribosome entry sequence (IRES). An IRES generally forms an RNA scaffold with precisely placed RNA tertiary structures that contact a 40S ribosomal subunit via a number of specific intermolecular interactions. Examples of ribosomal enhancer sequences are known and can be identified by the artisan (e.g., Mignone et al., Nucleic Acids Research 33: D141-D146 (2005); Paulous et al., Nucleic Acids Research 31: 722-733 (2003); Akbergenov et al., Nucleic Acids Research 32: 239-247 (2004); Mignone et al., Genome Biology 3(3): reviews0004.1-0001.10 (2002); Gallie, Nucleic Acids Research 30: 3401-3411 (2002); Shaloiko et al., http address at world wide web uniform resource locator interscience.wiley.com, DOI: 10.1002/bit.20267; and Gallie et al., Nucleic Acids Research 15: 3257-3273 (1987)).

A translational enhancer sequence sometimes is a eukaryotic sequence, such as a Kozak consensus sequence or other sequence (e.g., hydroid polyp sequence, GenBank accession no. U07128). A translational enhancer sequence sometimes is a prokaryotic sequence, such as a Shine-Dalgarno consensus sequence. In certain embodiments, the translational enhancer sequence is a viral nucleotide sequence. A translational enhancer sequence sometimes is from a 5′ UTR of a plant virus, such as Tobacco Mosaic Virus (TMV), Alfalfa Mosaic Virus (AMV); Tobacco Etch Virus (ETV); Potato Virus Y (PVY); Turnip Mosaic (poty) Virus and Pea Seed Borne Mosaic Virus, for example. In certain embodiments, an omega sequence about 67 bases in length from TMV is included in the nucleic acid reagent as a translational enhancer sequence (e.g., devoid of guanosine nucleotides and includes a 25 nucleotide long poly (CAA) central region).

A 3′ UTR may comprise one or more elements endogenous to the nucleotide sequence from which it originates and sometimes includes one or more exogenous elements. A 3′ UTR may originate from any suitable nucleic acid, such as genomic DNA, plasmid DNA, RNA, or mRNA, for example, from any suitable organism (e.g., a virus, bacterium, yeast, fungi, plant, insect, or mammal). The artisan can select appropriate elements for the 3′ UTR based upon the chosen expression system (e.g., expression in a chosen organism, for example). A 3′ UTR sometimes comprises one or more of the following elements known to the artisan: transcription regulation site, transcription initiation site, transcription termination site, transcription factor binding site, translation regulation site, translation termination site, translation initiation site, translation factor binding site, ribosome binding site, replicon, enhancer element, silencer element and polyadenosine tail. A 3′ UTR often includes a polyadenosine tail and sometimes does not, and if a polyadenosine tail is present, one or more adenosine moieties may be added or deleted from it (e.g., about 5, about 10, about 15, about 20, about 25, about 30, about 35, about 40, about 45 or about 50 adenosine moieties may be added or subtracted).

In some embodiments, modification of a 5′ UTR and/or a 3′ UTR can be used to alter (e.g., increase, add, decrease, or substantially eliminate) the activity of a promoter. Alteration of the promoter activity can in turn alter the activity of a peptide, polypeptide, or protein (e.g., enzyme activity for example), by a change in transcription of the nucleotide sequence(s) of interest from an operably linked promoter element comprising the modified 5′ or 3′ UTR. For example, a microorganism can be engineered by genetic modification to express a nucleic acid reagent comprising a modified 5′ or 3′ UTR that can add a novel activity (e.g., an activity not normally found in the host organism) or increase the expression of an existing activity by increasing transcription from a homologous or heterologous promoter operably linked to a nucleotide sequence of interest (e.g., homologous or heterologous nucleotide sequence of interest), in certain embodiments. In some embodiments, a microorganism can be engineered by genetic modification to express a nucleic acid reagent comprising a modified 5′ or 3′ UTR that can decrease the expression of an activity by decreasing or substantially eliminating transcription from a homologous or heterologous promoter operably linked to a nucleotide sequence of interest, in certain embodiments.

A nucleotide reagent sometimes can comprise a target nucleotide sequence. A “target nucleotide sequence” as used herein encodes a nucleic acid, peptide, polypeptide, or protein of interest, and may be a ribonucleotide sequence or a deoxyribonucleotide sequence.

A target nucleic acid sometimes can comprise a chimeric nucleic acid (or chimeric nucleotide sequence), which can encode a chimeric protein (or chimeric amino acid sequence). The term “chimeric” as used herein refers to a nucleic acid or nucleotide sequence, or encoded product thereof, containing sequences from two or more different sources. Any suitable source can be selected, including, but not limited to, a sequence from a nucleic acid, nucleotide sequence, ribosomal nucleic acid, RNA, DNA, regulatory nucleotide sequence (e.g., promoter, URL, enhancer, repressor, and the like), coding nucleic acid, gene, nucleic acid linker, nucleic acid tag, amino acid sequence, peptide, polypeptide, protein, chromosome, and organism. A chimeric molecule can include a sequence of contiguous nucleotides or amino acids from a source including, but not limited to, a virus, prokaryote, eukaryote, genus, species, homolog, ortholog, paralog and isozyme, nucleic acid linkers, nucleic acid tags, the like and combinations thereof). A chimeric molecule can be generated by placing in juxtaposition fragments of related or unrelated nucleic acids, nucleotide sequences or DNA segments, in some embodiments. In certain embodiments the nucleic acids, nucleotide sequences or DNA segments can be native or wild type sequences, mutant sequences or engineered sequences (completely engineered or engineered to a point, for example).

In some embodiments, a chimera includes about 1, 2, 3, 4 or 5 sequences (e.g., contiguous nucleotides, contiguous amino acids) from one organism and 1, 2, 3, 4 or 5 sequences (e.g., contiguous nucleotides, contiguous amino acids) from another organism. The organisms sometimes are a microbe, such as a bacterium (e.g., gram positive, gram negative), yeast or fungus (e.g., aerobic fungus, anaerobic fungus), for example. In some embodiments, the organisms are bacteria, the organisms are yeast or the organisms are fungi (e.g., different species), and sometimes one organism is a bacterium or yeast and another is a fungus. A chimeric molecule may contain up to about 99% of sequences from one organism (e.g., about 1, 2, 3, 4, 5, 6, 7, 8, 9, 10, 15, 20, 25, 30, 35, 40, 45, 50, 55, 60, 65, 70, 75, 80, 85, 90, 95, 96, 97, 98, 99%) and the balance percentage from one or more other organisms. In certain embodiments, a chimeric molecule includes altered codons (in the case of a chimeric nucleic acid) and one or more mutations (e.g., point mutations, nucleotide substitutions, amino acid substitutions).

A chimera sometimes is the result of recombination between two or more nucleic acids, nucleotide sequences or genes, and sometimes is the result of genetic manipulation (e.g., designed and/or generated by the hand of a human being). Any suitable nucleic acid or nucleotide sequence and method for combining nucleic acids or nucleotide sequences can be used to generate a chimeric nucleic acid or nucleotide sequence. Non-limiting examples of nucleic acid and nucleotide sequence sources and methods for generating chimeric nucleic acids and nucleotide sequences are presented herein.

In some embodiments, fragments used to generate a chimera can be juxtaposed as units (e.g., nucleic acid from the sources are combined end to end and not interspersed. In embodiments where a chimera includes one stretch of contiguous nucleotides for each organism, nucleotide sequence combinations can be noted as DNA source 1 DNA source 2 or DNA source 1/DNA source 2/DNA source 3, the like and combinations thereof, for example. In certain embodiments, fragments used to generate a chimera can be juxtaposed such that one or more fragments from one or more sources can be interspersed with other fragments used to generate the chimera (e.g., DNA source 1/DNA source 2/DNA source 1/DNA source 3/DNA source 2/DNA source 1). In some embodiments, the nucleotide sequence length of the fragments used to generate a chimera can be in the range from about 5 base pairs to about 5,000 base pairs (e.g., about 5 base pairs (bp), about 10 bp, about 15 bp, about 20 bp, about 25 bp, about 30 bp, about 35 bp, about 40 bp, about 45 bp, about 50 bp, about 55 bp, about 60 bp, about bp, about 65 bp, about 70 bp, about 75 bp, about 80 bp, about 85 bp, about 90 bp, about 95 bp, about 100 bp, about 125 bp, about 150 bp, about 175 bp, about 200 bp, about 250 bp, about 300 bp, about 350 bp, about 400 bp, about 450 bp, about 500 bp, about 550 bp, about 600 bp, about 650 bp, about 700 bp, about 750 bp, about 800 bp, about 850 bp, about 900 bp, about 950 bp, about 1000 bp, about 1500 bp, about 2000 bp, about 2500 bp, about 3000 bp, about 3500 bp, about 4000 bp, about 4500 bp, or about 5000 bp).

In certain embodiments, a chimeric nucleic acid or nucleotide sequence encodes the same activity as the activity encoded by the source nucleic acids or nucleotide sequences. In some embodiments, a chimeric nucleic acid or nucleotide sequence has a similar or the same activity, but the amount of the activity, or kinetics of the activity, are altered (e.g., increased, decreased). In certain embodiments, a chimeric nucleic acid or nucleotide sequence encodes a different activity, and in some embodiments a chimeric nucleic acid or nucleotide sequences encodes a chimeric activity (e.g., a combination of two or more activities).

A target nucleic acid sometimes is an untranslated ribonucleic acid and sometimes is a translated ribonucleic acid. An untranslated ribonucleic acid may include, but is not limited to, a small interfering ribonucleic acid (siRNA), a short hairpin ribonucleic acid (shRNA), other ribonucleic acid capable of RNA interference (RNAi), an antisense ribonucleic acid, or a ribozyme. A translatable target nucleotide sequence (e.g., a target ribonucleotide sequence) sometimes encodes a peptide, polypeptide, or protein, which are sometimes referred to herein as “target peptides,” “target polypeptides” or “target proteins.”

Any peptides, polypeptides or proteins, or an activity catalyzed by one or more peptides, polypeptides or proteins may be encoded by a target nucleotide sequence and may be selected by a person of ordinary skill in the art. Representative proteins include enzymes (e.g., phosphofructokinase activity, phosphogluconate dehydratase activity, 2-keto-3-deoxygluconate-6-phosphate aldolase activity, xylose isomerase activity, phosphoenolpyruvate carboxylase activity, alcohol dehydrogenase 2 activity and thymidylate synthase activity and the like, for example), antibodies, serum proteins (e.g., albumin), membrane bound proteins, hormones (e.g., growth hormone, erythropoietin, insulin, etc.), cytokines, etc., and include both naturally occurring and exogenously expressed polypeptides. Representative activities (e.g., enzymes or combinations of enzymes which are functionally associated to provide an activity) include phosphofructokinase activity, phosphogluconate dehydratase activity, 2-keto-3-deoxygluconate-6-phosphate aldolase activity, xylose isomerase activity, phosphoenolpyruvate carboxylase activity, alcohol dehydrogenase 2 activity and thymidylate synthase activity and the like for example. The term “enzyme” as used herein refers to a protein which can act as a catalyst to induce a chemical change in other compounds, thereby producing one or more products from one or more substrates.

Specific polypeptides (e.g., enzymes) useful for embodiments described herein are listed hereafter. The term “protein” as used herein refers to a molecule having a sequence of amino acids linked by peptide bonds. This term includes fusion proteins, oligopeptides, peptides, cyclic peptides, polypeptides, and polypeptide derivatives, whether native or recombinant, and also includes fragments, derivatives, homologs, and variants thereof. A protein or polypeptide sometimes is of intracellular origin (e.g., located in the nucleus, cytosol, or interstitial space of host cells in vivo) and sometimes is a cell membrane protein in vivo. In some embodiments (described above, and in further detail below in Engineering and Alteration Methods), a genetic modification can result in a modification (e.g., increase, substantially increase, decrease, or substantially decrease) of a target activity.

A translatable nucleotide sequence generally is located between a start codon (AUG in ribonucleic acids and ATG in deoxyribonucleic acids) and a stop codon (e.g., UAA (ochre), UAG (amber) or UGA (opal) in ribonucleic acids and TAA, TAG or TGA in deoxyribonucleic acids), and sometimes is referred to herein as an “open reading frame” (ORF). A nucleic acid reagent sometimes comprises one or more ORFs. An ORF may be from any suitable source, sometimes from genomic DNA, mRNA, reverse transcribed RNA, or complementary DNA (cDNA) or a nucleic acid library comprising one or more of the foregoing and is from any organism species that contains a nucleic acid sequence of interest, protein of interest, or activity of interest. Non-limiting examples of organisms from which an ORF can be obtained include bacteria, yeast, fungi, human, insect, nematode, bovine, equine, canine, feline, rat, or mouse, for example.

A nucleic acid reagent sometimes comprises a nucleotide sequence adjacent to an ORF that is translated in conjunction with the ORF and encodes an amino acid tag. The tag-encoding nucleotide sequence is located 3′ and/or 5′ of an ORF in the nucleic acid reagent, thereby encoding a tag at the C-terminus or N-terminus of the protein or peptide encoded by the ORF. Any tag that does not abrogate in vitro transcription and/or translation may be utilized and may be appropriately selected by the artisan. Tags may facilitate isolation and/or purification of the desired ORF product from culture or fermentation media.

A tag sometimes specifically binds a molecule or moiety of a solid phase or a detectable label, for example, thereby having utility for isolating, purifying and/or detecting a protein or peptide encoded by the ORF. In some embodiments, a tag comprises one or more of the following elements: FLAG (e.g., DYKDDDDKG (SEQ ID NO: 79)), V5 (e.g., GKPIPNPLLGLDST (SEQ ID NO: 80)), c-MYC (e.g., EQKLISEEDL (SEQ ID NO: 81)), HSV (e.g., QPELAPEDPED (SEQ ID NO: 82)), influenza hemaglutinin, HA (e.g., YPYDVPDYA (SEQ ID NO: 83)), VSV-G (e.g., YTDIEMNRLGK (SEQ ID NO: 182)), bacterial glutathione-S-transferase, maltose binding protein, a streptavidin- or avidin-binding tag (e.g., pcDNA™6 BioEase™ Gateway® Biotinylation System (Invitrogen)), thioredoxin, β-galactosidase, VSV-glycoprotein, a fluorescent protein (e.g., green fluorescent protein or one of its many color variants (e.g., yellow, red, blue)), a polylysine or polyarginine sequence, a polyhistidine sequence (e.g., His6 (SEQ ID NO: 84)) or other sequence that chelates a metal (e.g., cobalt, zinc, copper), and/or a cysteine-rich sequence that binds to an arsenic-containing molecule. In certain embodiments, a cysteine-rich tag comprises the amino acid sequence CC-Xn-CC (SEQ ID NO: 85), wherein X is any amino acid and n is 1 to 3, and the cysteine-rich sequence sometimes is CCPGCC (SEQ ID NO: 86). In certain embodiments, the tag comprises a cysteine-rich element and a polyhistidine element (e.g., CCPGCC (SEQ ID NO: 87) and His6 (SEQ ID NO: 88)).

A tag often conveniently binds to a binding partner. For example, some tags bind to an antibody (e.g., FLAG) and sometimes specifically bind to a small molecule. For example, a polyhistidine tag specifically chelates a bivalent metal, such as copper, zinc, and cobalt; a polylysine or polyarginine tag specifically binds to a zinc finger; a glutathione S-transferase tag binds to glutathione; and a cysteine-rich tag specifically binds to an arsenic-containing molecule. Arsenic-containing molecules include LUMIO™ agents (Invitrogen, California), such as FIAsH™ (EDT2[4′,5′-bis(1,3,2-dithioarsolan-2-yl)fluorescein-(1,2-ethanedithiol)2]) and ReAsH reagents (e.g., U.S. Pat. No. 5,932,474 to Tsien et al., entitled “Target Sequences for Synthetic Molecules;” U.S. Pat. No. 6,054,271 to Tsien et al., entitled “Methods of Using Synthetic Molecules and Target Sequences;” U.S. Pat. Nos. 6,451,569 and 6,008,378; published U.S. Patent Application 2003/0083373, and published PCT Patent Application WO 99/21013, all to Tsien et al. and all entitled “Synthetic Molecules that Specifically React with Target Sequences”). Such antibodies and small molecules sometimes are linked to a solid phase for convenient isolation of the target protein or target peptide.

A tag sometimes comprises a sequence that localizes a translated protein or peptide to a component in a system, which is referred to as a “signal sequence” or “localization signal sequence” herein. A signal sequence often is incorporated at the N-terminus of a target protein or target peptide, and sometimes is incorporated at the C-terminus. Examples of signal sequences are known to the artisan, are readily incorporated into a nucleic acid reagent, and often are selected according to the organism in which expression of the nucleic acid reagent is performed. A signal sequence in some embodiments localizes a translated protein or peptide to a cell membrane. Examples of signal sequences include, but are not limited to, a nucleus targeting signal (e.g., steroid receptor sequence and N-terminal sequence of SV40 virus large T antigen); mitochondrial targeting signal (e.g., amino acid sequence that forms an amphipathic helix); peroxisome targeting signal (e.g., C-terminal sequence in YFG from S. cerevisiae); and a secretion signal (e.g., N-terminal sequences from invertase, mating factor alpha, PHO5 and SUC2 in S. cerevisiae; multiple N-terminal sequences of B. subtilis proteins (e.g., Tjalsma et al., Microbiol. Molec. Biol. Rev. 64: 515-547 (2000)); alpha amylase signal sequence (e.g., U.S. Pat. No. 6,288,302); pectate lyase signal sequence (e.g., U.S. Pat. No. 5,846,818); precollagen signal sequence (e.g., U.S. Pat. No. 5,712,114); OmpA signal sequence (e.g., U.S. Pat. No. 5,470,719); lam beta signal sequence (e.g., U.S. Pat. No. 5,389,529); B. brevis signal sequence (e.g., U.S. Pat. No. 5,232,841); and P. pastoris signal sequence (e.g., U.S. Pat. No. 5,268,273)).

A tag sometimes is directly adjacent to the amino acid sequence encoded by an ORF (i.e., there is no intervening sequence) and sometimes a tag is substantially adjacent to an ORF encoded amino acid sequence (e.g., an intervening sequence is present). An intervening sequence sometimes includes a recognition site for a protease, which is useful for cleaving a tag from a target protein or peptide. In some embodiments, the intervening sequence is cleaved by Factor Xa (e.g., recognition site I (E/D)GR), thrombin (e.g., recognition site LVPRGS (SEQ ID NO: 89)), enterokinase (e.g., recognition site DDDDK (SEQ ID NO: 90)), TEV protease (e.g., recognition site ENLYFQG (SEQ ID NO: 91)) or PreScission™ protease (e.g., recognition site LEVLFQGP (SEQ ID NO: 92)), for example.

An intervening sequence sometimes is referred to herein as a “linker sequence,” and may be of any suitable length selected by the artisan. A linker sequence sometimes is about 1 to about 20 amino acids in length, and sometimes about 5 to about 10 amino acids in length. The artisan may select the linker length to substantially preserve target protein or peptide function (e.g., a tag may reduce target protein or peptide function unless separated by a linker), to enhance disassociation of a tag from a target protein or peptide when a protease cleavage site is present (e.g., cleavage may be enhanced when a linker is present), and to enhance interaction of a tag/target protein product with a solid phase. A linker can be of any suitable amino acid content, and often comprises a higher proportion of amino acids having relatively short side chains (e.g., glycine, alanine, serine, and threonine).

A nucleic acid reagent sometimes includes a stop codon between a tag element and an insertion element or ORF, which can be useful for translating an ORF with or without the tag. Mutant tRNA molecules that recognize stop codons (described above) suppress translation termination and thereby are designated “suppressor tRNAs.” Suppressor tRNAs can result in the insertion of amino acids and continuation of translation past stop codons (e.g., U.S. Patent Application No. 60/587,583, filed Jul. 14, 2004, entitled “Production of Fusion Proteins by Cell-Free Protein Synthesis,”; Eggertsson, et al., (1988) Microbiological Review 52(3):354-374, and Engleerg-Kukla, et al. (1996) in Escherichia coli and Salmonella Cellular and Molecular Biology, Chapter 60, pps 909-921, Neidhardt, et al. eds., ASM Press, Washington, D.C.). A number of suppressor tRNAs are known, including but not limited to, supE, supP, supD, supF and supZ suppressors, which suppress the termination of translation of the amber stop codon; supB, glT, supL, supN, supC and supM suppressors, which suppress the function of the ochre stop codon and glyT, trpT and Su-9 suppressors, which suppress the function of the opal stop codon. In general, suppressor tRNAs contain one or more mutations in the anti-codon loop of the tRNA that allows the tRNA to base pair with a codon that ordinarily functions as a stop codon. The mutant tRNA is charged with its cognate amino acid residue and the cognate amino acid residue is inserted into the translating polypeptide when the stop codon is encountered. Mutations that enhance the efficiency of termination suppressors (i.e., increase stop codon read-through) have been identified. These include, but are not limited to, mutations in the uar gene (also known as the prfA gene), mutations in the ups gene, mutations in the sueA, sueB and sueC genes, mutations in the rpsD (ramA) and rpsE (spcA) genes and mutations in the rplL gene.

Thus, a nucleic acid reagent comprising a stop codon located between an ORF and a tag can yield a translated ORF alone when no suppressor tRNA is present in the translation system and can yield a translated ORF-tag fusion when a suppressor tRNA is present in the system. Suppressor tRNA can be generated in cells transfected with a nucleic acid encoding the tRNA (e.g., a replication incompetent adenovirus containing the human tRNA-Ser suppressor gene can be transfected into cells, or a YAC containing a yeast or bacterial tRNA suppressor gene can be transfected into yeast cells, for example). Vectors for synthesizing suppressor tRNA and for translating ORFs with or without a tag are available to the artisan (e.g., Tag-On-Demand™ kit (Invitrogen Corporation, California); Tag-On-Demand™ Suppressor Supernatant Instruction Manual, Version B, 6 Jun. 2003, at http address at world wide web uniform resource locator invitrogen.com/content/sfs/manuals/tagondemand_supernatant_man.pdf; Tag-On-Demand™ Gateway® Vector Instruction Manual, Version B, 20 Jun. 2003 at http address at world wide web uniform resource locator invitrogen.com/content/sfs/manuals/tagondemand_vectors_man.pdf; and Capone et al., Amber, ochre and opal suppressor tRNA genes derived from a human serine tRNA gene. EMBO J. 4:213, 1985).

Any convenient cloning strategy known in the art may be utilized to incorporate an element, such as an ORF, into a nucleic acid reagent. Known methods can be utilized to insert an element into the template independent of an insertion element, such as (1) cleaving the template at one or more existing restriction enzyme sites and ligating an element of interest and (2) adding restriction enzyme sites to the template by hybridizing oligonucleotide primers that include one or more suitable restriction enzyme sites and amplifying by polymerase chain reaction (described in greater detail herein). Other cloning strategies take advantage of one or more insertion sites present or inserted into the nucleic acid reagent, such as an oligonucleotide primer hybridization site for PCR, for example, and others described hereafter. In some embodiments, a cloning strategy can be combined with genetic manipulation such as recombination (e.g., recombination of a nucleic acid reagent with a nucleic acid sequence of interest into the genome of the organism to be modified, as described further below). In some embodiments, the cloned ORF(s) can produce (directly or indirectly) a desired product, by engineering a microorganism with one or more ORFs of interest, which microorganism comprises one or more altered activities selected from the group consisting of phosphofructokinase activity, phosphogluconate dehydratase activity, 2-keto-3-deoxygluconate-6-phosphate aldolase activity, xylose isomerase activity, phosphoenolpyruvate carboxylase activity, alcohol dehydrogenase 2 activity, sugar transport activity, phosphoglucoisomerase activity, transaldolase activity, transketolase activity, glucose-6-phosphate dehydrogenase activity, 6-phosphogluconolactonase activity, 6-phosphogluconate dehydrogenase (decarboxylating) activity, and thymidylate synthase activity.

In some embodiments, the nucleic acid reagent includes one or more recombinase insertion sites. A recombinase insertion site is a recognition sequence on a nucleic acid molecule that participates in an integration/recombination reaction by recombination proteins. For example, the recombination site for Cre recombinase is IoxP, which is a 34 base pair sequence comprised of two 13 base pair inverted repeats (serving as the recombinase binding sites) flanking an 8 base pair core sequence (e.g., FIG. 1 of Sauer, B., Curr. Opin. Biotech. 5:521-527 (1994)). Other examples of recombination sites include attB, attP, attL, and attR sequences, and mutants, fragments, variants and derivatives thereof, which are recognized by the recombination protein λ Int and by the auxiliary proteins integration host factor (IHF), FIS and excisionase (Xis) (e.g., U.S. Pat. Nos. 5,888,732; 6,143,557; 6,171,861; 6,270,969; 6,277,608; and 6,720,140; U.S. patent application Ser. No. 09/517,466, filed Mar. 2, 2000, and Ser. No. 09/732,914, filed Aug. 14, 2003, and in U.S. patent publication no. 2002-0007051-A1; Landy, Curr. Opin. Biotech. 3:699-707 (1993)).

Examples of recombinase cloning nucleic acids are in Gateway® systems (Invitrogen, California), which include at least one recombination site for cloning a desired nucleic acid molecules in vivo or in vitro. In some embodiments, the system utilizes vectors that contain at least two different site-specific recombination sites, often based on the bacteriophage lambda system (e.g., att1 and att2), and are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the Gateway® system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

A recombination system useful for engineering yeast is outlined briefly. The system makes use of the ura3 gene (e.g., for S. cerevisiae and C. albicans, for example) or ura4 and ura5 genes (e.g., for S. pombe, for example) and toxicity of the nucleotide analogue 5-Fluoroorotic acid (5-FOA). The ura3 or ura4 and ura5 genes encode orotine-5′-monophosphate (OMP) dicarboxylase. Yeast with an active ura3 or ura4 and ura5 gene (phenotypically Ura+) convert 5-FOA to fluorodeoxyuridine, which is toxic to yeast cells. Yeast carrying a mutation in the appropriate gene(s) or having a knock out of the appropriate gene(s) can grow in the presence of 5-FOA, if the media is also supplemented with uracil.

A nucleic acid engineering construct can be made which may comprise the URA3 gene or cassette (for S. cerevisiae), flanked on either side by the same nucleotide sequence in the same orientation. The ura3 cassette comprises a promoter, the ura3 gene and a functional transcription terminator. Target sequences which direct the construct to a particular nucleic acid region of interest in the organism to be engineered are added such that the target sequences are adjacent to and abut the flanking sequences on either side of the ura3 cassette. Yeast can be transformed with the engineering construct and plated on minimal media without uracil. Colonies can be screened by PCR to determine those transformants that have the engineering construct inserted in the proper location in the genome. Checking insertion location prior to selecting for recombination of the ura3 cassette may reduce the number of incorrect clones carried through to later stages of the procedure. Correctly inserted transformants can then be replica plated on minimal media containing 5-FOA to select for recombination of the ura3 cassette out of the construct, leaving a disrupted gene and an identifiable footprint (e.g., nucleic acid sequence) that can be use to verify the presence of the disrupted gene. The technique described is useful for disrupting or “knocking out” gene function, but also can be used to insert genes or constructs into a host organisms genome in a targeted, sequence specific manner. Further detail will be described below in the engineering section and in the example section.

In certain embodiments, a nucleic acid reagent includes one or more topoisomerase insertion sites. A topoisomerase insertion site is a defined nucleotide sequence recognized and bound by a site-specific topoisomerase. For example, the nucleotide sequence 5′-(C/T)CCTT-3′ is a topoisomerase recognition site bound specifically by most poxvirus topoisomerases, including vaccinia virus DNA topoisomerase I. After binding to the recognition sequence, the topoisomerase cleaves the strand at the 3′-most thymidine of the recognition site to produce a nucleotide sequence comprising 5′-(C/T)CCTT-PO4-TOPO, a complex of the topoisomerase covalently bound to the 3′ phosphate via a tyrosine in the topoisomerase (e.g., Shuman, J. Biol. Chem. 266:11372-11379, 1991; Sekiguchi and Shuman, Nucl. Acids Res. 22:5360-5365, 1994; U.S. Pat. No. 5,766,891; PCT/US95/16099; and PCT/US98/12372). In comparison, the nucleotide sequence 5′-GCAACTT-3′ is a topoisomerase recognition site for type IA E. coli topoisomerase III. An element to be inserted often is combined with topoisomerase-reacted template and thereby incorporated into the nucleic acid reagent (e.g., http address at world wide web uniform resource locator invitrogen.com/downloads/F-13512_Topo_Flyer.pdf; http address at world wide web uniform resource locator invitrogen.com/content/sfs/brochures/710_021849%20_B_TOPOCloning_bro.pdf; TOPO TA Cloning® Kit and Zero Blunt® TOPO® Cloning Kit product information).

A nucleic acid reagent sometimes contains one or more origin of replication (ORI) elements. In some embodiments, a template comprises two or more ORIs, where one functions efficiently in one organism (e.g., a bacterium) and another functions efficiently in another organism (e.g., a eukaryote, like yeast for example). In some embodiments, an ORI may function efficiently in one species (e.g., S. cerevisiae, for example) and another ORI may function efficiently in a different species (e.g., S. pombe, for example). A nucleic acid reagent also sometimes includes one or more transcription regulation sites.

A nucleic acid reagent can include one or more selection elements (e.g., elements for selection of the presence of the nucleic acid reagent, and not for activation of a promoter element which can be selectively regulated). Selection elements often are utilized using known processes to determine whether a nucleic acid reagent is included in a cell. In some embodiments, a nucleic acid reagent includes two or more selection elements, where one functions efficiently in one organism and another functions efficiently in another organism. Examples of selection elements include, but are not limited to, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like).

A nucleic acid reagent is of any form useful for in vivo transcription and/or translation. A nucleic acid sometimes is a plasmid, such as a supercoiled plasmid, sometimes is a yeast artificial chromosome (e.g., YAC), sometimes is a linear nucleic acid (e.g., a linear nucleic acid produced by PCR or by restriction digest), sometimes is single-stranded and sometimes is double-stranded. A nucleic acid reagent sometimes is prepared by an amplification process, such as a polymerase chain reaction (PCR) process or transcription-mediated amplification process (TMA). In TMA, two enzymes are used in an isothermal reaction to produce amplification products detected by light emission (see, e.g., Biochemistry 1996 Jun. 25; 35(25):8429-38 and http address world wide web uniform resource locator devicelink.com/ivdt/archive/00/11/007.html). Standard PCR processes are known (e.g., U.S. Pat. Nos. 4,683,202; 4,683,195; 4,965,188; and 5,656,493), and generally are performed in cycles. Each cycle includes heat denaturation, in which hybrid nucleic acids dissociate; cooling, in which primer oligonucleotides hybridize; and extension of the oligonucleotides by a polymerase (i.e., Taq polymerase). An example of a PCR cyclical process is treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Multiple cycles frequently are performed using a commercially available thermal cycler. PCR amplification products sometimes are stored for a time at a lower temperature (e.g., at 4° C.) and sometimes are frozen (e.g., at −20° C.) before analysis.

In some embodiments, a nucleic acid reagent, protein reagent, protein fragment reagent or other reagent described herein is isolated or purified. The term “isolated” as used herein refers to material removed from its original environment (e.g., the natural environment if it is naturally occurring, or a host cell if expressed exogenously), and thus is altered “by the hand of man” from its original environment. The term “purified” as used herein with reference to molecules does not refer to absolute purity. Rather, “purified” refers to a substance in a composition that contains fewer substance species in the same class (e.g., nucleic acid or protein species) other than the substance of interest in comparison to the sample from which it originated. “Purified,” if a nucleic acid or protein for example, refers to a substance in a composition that contains fewer nucleic acid species or protein species other than the nucleic acid or protein of interest in comparison to the sample from which it originated. Sometimes, a protein or nucleic acid is “substantially pure,” indicating that the protein or nucleic acid represents at least 50% of protein or nucleic acid on a mass basis of the composition. Often, a substantially pure protein or nucleic acid is at least 75% on a mass basis of the composition, and sometimes at least 95% on a mass basis of the composition.

### Engineering and Alteration Methods

Methods and compositions (e.g., nucleic acid reagents) described herein can be used to generate engineered microorganisms. As noted above, the term “engineered microorganism” as used herein refers to a modified organism that includes one or more activities distinct from an activity present in a microorganism utilized as a starting point for modification (e.g., host microorganism or unmodified organism). Engineered microorganisms typically arise as a result of a genetic modification, usually introduced or selected for, by one of skill in the art using readily available techniques. Non-limiting examples of methods useful for generating an altered activity include, introducing a heterologous polynucleotide (e.g., nucleic acid or gene integration, also referred to as “knock in”), removing an endogenous polynucleotide, altering the sequence of an existing endogenous nucleic acid sequence (e.g., site-directed mutagenesis), disruption of an existing endogenous nucleic acid sequence (e.g., knock outs and transposon or insertion element mediated mutagenesis), selection for an altered activity where the selection causes a change in a naturally occurring activity that can be stably inherited (e.g., causes a change in a nucleic acid sequence in the genome of the organism or in an epigenetic nucleic acid that is replicated and passed on to daughter cells), PCR-based mutagenesis, and the like. The term “mutagenesis” as used herein refers to any modification to a nucleic acid (e.g., nucleic acid reagent, or host chromosome, for example) that is subsequently used to generate a product in a host or modified organism. Non-limiting examples of mutagenesis include deletion, insertion, substitution, rearrangement, point mutations, suppressor mutations and the like. Mutagenesis methods are known in the art and are readily available to the artisan. Non-limiting examples of mutagenesis methods are described herein and can also be found in Maniatis, T., E. F. Fritsch and J. Sambrook (1982) Molecular Cloning: a Laboratory Manual; Cold Spring Harbor Laboratory, Cold Spring Harbor, N.Y.

The term “genetic modification” as used herein refers to any suitable nucleic acid addition, removal or alteration that facilitates production of a target product (e.g., phosphogluconate dehydratase activity, 2-keto-3-deoxygluconate-6-phosphate aldolase activity, xylose isomerase activity, or phosphoenolpyruvate carboxylase activity, for example). in an engineered microorganism. Genetic modifications include, without limitation, insertion of one or more nucleotides in a native nucleic acid of a host organism in one or more locations, deletion of one or more nucleotides in a native nucleic acid of a host organism in one or more locations, modification or substitution of one or more nucleotides in a native nucleic acid of a host organism in one or more locations, insertion of a non-native nucleic acid into a host organism (e.g., insertion of an autonomously replicating vector), and removal of a non-native nucleic acid in a host organism (e.g., removal of a vector).

The term “heterologous polynucleotide” as used herein refers to a nucleotide sequence not present in a host microorganism in some embodiments. In certain embodiments, a heterologous polynucleotide is present in a different amount (e.g., different copy number) than in a host microorganism, which can be accomplished, for example, by introducing more copies of a particular nucleotide sequence to a host microorganism (e.g., the particular nucleotide sequence may be in a nucleic acid autonomous of the host chromosome or may be inserted into a chromosome). A heterologous polynucleotide is from a different organism in some embodiments, and in certain embodiments, is from the same type of organism but from an outside source (e.g., a recombinant source).

The term “altered activity” as used herein refers to an activity in an engineered microorganism that is added or modified relative to the host microorganism (e.g., added, increased, reduced, inhibited, or removed activity). An activity can be altered by introducing a genetic modification to a host microorganism that yields an engineered microorganism having added, increased, reduced, inhibited, or removed activity.

An added activity often is an activity not detectable in a host microorganism. An increased activity generally is an activity detectable in a host microorganism that has been increased in an engineered microorganism. An activity can be increased to any suitable level for production of a target product (e.g., adipic acid, 6-hydroxyhexanoic acid), including but not limited to less than 2-fold (e.g., about 10% increase to about 99% increase; about 20%, 30%, 40%, 50%, 60%, 70%, 80%, 90% increase), 2-fold, 3-fold, 4-fold, 5-fold, 6-fold, 7-fold, 8-fold, 9-fold, of 10-fold increase, or greater than about 10-fold increase. A reduced or inhibited activity generally is an activity detectable in a host microorganism that has been reduced or inhibited in an engineered microorganism. An activity can be reduced to undetectable levels in some embodiments, or detectable levels in certain embodiments. An activity can be decreased to any suitable level for production of a target product (e.g., adipic acid, 6-hydroxyhexanoic acid), including but not limited to less than 2-fold (e.g., about 10% decrease to about 99% decrease; about 20%, 30%, 40%, 50%, 60%, 70%, 80%, 90% decrease), 2-fold, 3-fold, 4-fold, 5-fold, 6-fold, 7-fold, 8-fold, 9-fold, of 10-fold decrease, or greater than about 10-fold decrease.

An altered activity sometimes is an activity not detectable in a host organism and is added to an engineered organism. An altered activity also may be an activity detectable in a host organism and is increased in an engineered organism. An activity may be added or increased by increasing the number of copies of a polynucleotide that encodes a polypeptide having a target activity, in some embodiments. In certain embodiments an activity can be added or increased by inserting into a host microorganism a heterologous polynucleotide that encodes a polypeptide having the added activity. In certain embodiments, an activity can be added or increased by inserting into a host microorganism a heterologous polynucleotide that is (i) operably linked to another polynucleotide that encodes a polypeptide having the added activity, and (ii) up regulates production of the polynucleotide. Thus, an activity can be added or increased by inserting or modifying a regulatory polynucleotide operably linked to another polynucleotide that encodes a polypeptide having the target activity. In certain embodiments, an activity can be added or increased by subjecting a host microorganism to a selective environment and screening for microorganisms that have a detectable level of the target activity. Examples of a selective environment include, without limitation, a medium containing a substrate that a host organism can process and a medium lacking a substrate that a host organism can process.

An altered activity sometimes is an activity detectable in a host organism and is reduced, inhibited, or removed (i.e., not detectable) in an engineered organism. An activity may be reduced or removed by decreasing the number of copies of a polynucleotide that encodes a polypeptide having a target activity, in some embodiments. In some embodiments, an activity can be reduced or removed by (i) inserting a polynucleotide within a polynucleotide that encodes a polypeptide having the target activity (disruptive insertion), and/or (ii) removing a portion of or all of a polynucleotide that encodes a polypeptide having the target activity (deletion or knock out, respectively). In certain embodiments, an activity can be reduced or removed by inserting into a host microorganism a heterologous polynucleotide that is (i) operably linked to another polynucleotide that encodes a polypeptide having the target activity, and (ii) down regulates production of the polynucleotide. Thus, an activity can be reduced or removed by inserting or modifying a regulatory polynucleotide operably linked to another polynucleotide that encodes a polypeptide having the target activity.

An activity also can be reduced or removed by (i) inhibiting a polynucleotide that encodes a polypeptide having the activity or (ii) inhibiting a polynucleotide operably linked to another polynucleotide that encodes a polypeptide having the activity. A polynucleotide can be inhibited by a suitable technique known in the art, such as by contacting an RNA encoded by the polynucleotide with a specific inhibitory RNA (e.g., RNAi, siRNA, ribozyme). An activity also can be reduced or removed by contacting a polypeptide having the activity with a molecule that specifically inhibits the activity (e.g., enzyme inhibitor, antibody). In certain embodiments, an activity can be reduced or removed by subjecting a host microorganism to a selective environment and screening for microorganisms that have a reduced level or removal of the target activity.

In some embodiments, an untranslated ribonucleic acid, or a cDNA can be used to reduce the expression of a particular activity or enzyme. For example, a microorganism can be engineered by genetic modification to express a nucleic acid reagent that reduces the expression of an activity by producing an RNA molecule that is partially or substantially homologous to a nucleic acid sequence of interest which encodes the activity of interest. The RNA molecule can bind to the nucleic acid sequence of interest and inhibit the nucleic acid sequence from performing its natural function, in certain embodiments. In some embodiments, the RNA may alter the nucleic acid sequence of interest which encodes the activity of interest in a manner that the nucleic acid sequence of interest is no longer capable of performing its natural function (e.g., the action of a ribozyme for example).

In certain embodiments, nucleotide sequences sometimes are added to, modified, or removed from one or more of the nucleic acid reagent elements, such as the promoter, 5′UTR, target sequence, or 3′UTR elements, to enhance, potentially enhance, reduce, or potentially reduce transcription and/or translation before or after such elements are incorporated in a nucleic acid reagent. In some embodiments, one or more of the following sequences may be modified or removed if they are present in a 5′UTR: a sequence that forms a stable secondary structure (e.g., quadruplex structure or stem loop stem structure (e.g., EMBL sequences X12949, AF274954, AF139980, AF152961, S95936, U194144, AF116649 or substantially identical sequences that form such stem loop stem structures)); a translation initiation codon upstream of the target nucleotide sequence start codon; a stop codon upstream of the target nucleotide sequence translation initiation codon; an ORF upstream of the target nucleotide sequence translation initiation codon; an iron responsive element (IRE) or like sequence; and a 5′ terminal oligopyrimidine tract (TOP, e.g., consisting of 5-15 pyrimidines adjacent to the cap). A translational enhancer sequence and/or an internal ribosome entry site (IRES) sometimes is inserted into a 5′UTR (e.g., EMBL nucleotide sequences J04513, X87949, M95825, M12783, AF025841, AF013263, AF006822, M17169, M13440, M22427, D14838 and M17446 and substantially identical nucleotide sequences).

An AU-rich element (ARE, e.g., AUUUA repeats) and/or splicing junction that follows a non-sense codon sometimes is removed from or modified in a 3′UTR. A polyadenosine tail sometimes is inserted into a 3′UTR if none is present, sometimes is removed if it is present, and adenosine moieties sometimes are added to or removed from a polyadenosine tail present in a 3′UTR. Thus, some embodiments are directed to a process comprising: determining whether any nucleotide sequences that increase, potentially increase, reduce, or potentially reduce translation efficiency are present in the elements, and adding, removing, or modifying one or more of such sequences if they are identified. Certain embodiments are directed to a process comprising: determining whether any nucleotide sequences that increase or potentially increase translation efficiency are not present in the elements, and incorporating such sequences into the nucleic acid reagent.

In some embodiments, an activity can be altered by modifying the nucleotide sequence of an ORF. An ORF sometimes is mutated or modified (for example, by point mutation, deletion mutation, insertion mutation, PCR based mutagenesis and the like) to alter, enhance or increase, reduce, substantially reduce, or eliminate the activity of the encoded protein or peptide. The protein or peptide encoded by a modified ORF sometimes is produced in a lower amount or may not be produced at detectable levels, and in other embodiments, the product or protein encoded by the modified ORF is produced at a higher level (e.g., codons sometimes are modified so they are compatible with tRNA's preferentially used in the host organism or engineered organism). To determine the relative activity, the activity from the product of the mutated ORF (or cell containing it) can be compared to the activity of the product or protein encoded by the unmodified ORF (or cell containing it).

In some embodiments, an ORF nucleotide sequence sometimes is mutated or modified to alter the triplet nucleotide sequences used to encode amino acids (e.g., amino acid codon triplets, for example). Modification of the nucleotide sequence of an ORF to alter codon triplets sometimes is used to change the codon found in the original sequence to better match the preferred codon usage of the organism in which the ORF or nucleic acid reagent will be expressed. For example, the codon usage, and therefore the codon triplets encoded by a nucleic acid sequence from bacteria may be different from the preferred codon usage in eukaryotes like yeast or plants. Preferred codon usage also may be different between bacterial species. In certain embodiments an ORF nucleotide sequences sometimes is modified to eliminate codon pairs and/or eliminate mRNA secondary structures that can cause pauses during translation of the mRNA encoded by the ORF nucleotide sequence. Translational pausing sometimes occurs when nucleic acid secondary structures exist in an mRNA, and sometimes occurs due to the presence of codon pairs that slow the rate of translation by causing ribosomes to pause. In some embodiments, the use of lower abundance codon triplets can reduce translational pausing due to a decrease in the pause time needed to load a charged tRNA into the ribosome translation machinery. Therefore, to increase transcriptional and translational efficiency in bacteria (e.g., where transcription and translation are concurrent, for example) or to increase translational efficiency in eukaryotes (e.g., where transcription and translation are functionally separated), the nucleotide sequence of a nucleotide sequence of interest can be altered to better suit the transcription and/or translational machinery of the host and/or genetically modified microorganism. In certain embodiment, slowing the rate of translation by the use of lower abundance codons, which slow or pause the ribosome, can lead to higher yields of the desired product due to an increase in correctly folded proteins and a reduction in the formation of inclusion bodies.

Codons can be altered and optimized according to the preferred usage by a given organism by determining the codon distribution of the nucleotide sequence donor organism and comparing the distribution of codons to the distribution of codons in the recipient or host organism. Techniques described herein (e.g., site directed mutagenesis and the like) can then be used to alter the codons accordingly. Comparisons of codon usage can be done by hand, or using nucleic acid analysis software commercially available to the artisan.

Modification of the nucleotide sequence of an ORF also can be used to correct codon triplet sequences that have diverged in different organisms. For example, certain yeast (e.g., C. tropicalis and C. maltosa) use the amino acid triplet CUG (e.g., CTG in the DNA sequence) to encode serine. CUG typically encodes leucine in most organisms. In order to maintain the correct amino acid in the resultant polypeptide or protein, the CUG codon must be altered to reflect the organism in which the nucleic acid reagent will be expressed. Thus, if an ORF from a bacterial donor is to be expressed in either Candida yeast strain mentioned above, the heterologous nucleotide sequence must first be altered or modified to the appropriate leucine codon. Therefore, in some embodiments, the nucleotide sequence of an ORF sometimes is altered or modified to correct for differences that have occurred in the evolution of the amino acid codon triplets between different organisms. In some embodiments, the nucleotide sequence can be left unchanged at a particular amino acid codon, if the amino acid encoded is a conservative or neutral change in amino acid when compared to the originally encoded amino acid.

In some embodiments, an activity can be altered by modifying translational regulation signals, like a stop codon for example. A stop codon at the end of an ORF sometimes is modified to another stop codon, such as an amber stop codon described above. In some embodiments, a stop codon is introduced within an ORF, sometimes by insertion or mutation of an existing codon. An ORF comprising a modified terminal stop codon and/or internal stop codon often is translated in a system comprising a suppressor tRNA that recognizes the stop codon. An ORF comprising a stop codon sometimes is translated in a system comprising a suppressor tRNA that incorporates an unnatural amino acid during translation of the target protein or target peptide. Methods for incorporating unnatural amino acids into a target protein or peptide are known, which include, for example, processes utilizing a heterologous tRNA/synthetase pair, where the tRNA recognizes an amber stop codon and is loaded with an unnatural amino acid (e.g., World Wide Web URL iupac.org/news/prize/2003/wang.pdf).

Depending on the portion of a nucleic acid reagent (e.g., Promoter, 5′ or 3′ UTR, ORI, ORF, and the like) chosen for alteration (e.g., by mutagenesis, introduction or deletion, for example) the modifications described above can alter a given activity by (i) increasing or decreasing feedback inhibition mechanisms, (ii) increasing or decreasing promoter initiation, (iii) increasing or decreasing translation initiation, (iv) increasing or decreasing translational efficiency, (v) modifying localization of peptides or products expressed from nucleic acid reagents described herein, or (vi) increasing or decreasing the copy number of a nucleotide sequence of interest, (vii) expression of an anti-sense RNA, RNAi, siRNA, ribozyme and the like. In some embodiments, alteration of a nucleic acid reagent or nucleotide sequence can alter a region involved in feedback inhibition (e.g., 5′ UTR, promoter, and the like). A modification sometimes is made that can add or enhance binding of a feedback regulator and sometimes a modification is made that can reduce, inhibit, or eliminate binding of a feedback regulator.

In certain embodiments, alteration of a nucleic acid reagent or nucleotide sequence can alter sequences involved in transcription initiation (e.g., promoters, 5′ UTR, and the like). A modification sometimes can be made that can enhance or increase initiation from an endogenous or heterologous promoter element. A modification sometimes can be made that removes or disrupts sequences that increase or enhance transcription initiation, resulting in a decrease or elimination of transcription from an endogenous or heterologous promoter element.

In some embodiments, alteration of a nucleic acid reagent or nucleotide sequence can alter sequences involved in translational initiation or translational efficiency (e.g., 5′ UTR, 3′ UTR, codon triplets of higher or lower abundance, translational terminator sequences and the like, for example). A modification sometimes can be made that can increase or decrease translational initiation, modifying a ribosome binding site for example. A modification sometimes can be made that can increase or decrease translational efficiency. Removing or adding sequences that form hairpins and changing codon triplets to a more or less preferred codon are non-limiting examples of genetic modifications that can be made to alter translation initiation and translation efficiency.

In certain embodiments, alteration of a nucleic acid reagent or nucleotide sequence can alter sequences involved in localization of peptides, proteins, or other desired products (e.g., adipic acid, for example). A modification sometimes can be made that can alter, add, or remove sequences responsible for targeting a polypeptide, protein or product to an intracellular organelle, the periplasm, cellular membranes, or extracellularly. Transport of a heterologous product to a different intracellular space or extracellularly sometimes can reduce or eliminate the formation of inclusion bodies (e.g., insoluble aggregates of the desired product).

In some embodiments, alteration of a nucleic acid reagent or nucleotide sequence can alter sequences involved in increasing or decreasing the copy number of a nucleotide sequence of interest. A modification sometimes can be made that increases or decreases the number of copies of an ORF stably integrated into the genome of an organism or on an epigenetic nucleic acid reagent. Non-limiting examples of alterations that can increase the number of copies of a sequence of interest include, adding copies of the sequence of interest by duplication of regions in the genome (e.g., adding additional copies by recombination or by causing gene amplification of the host genome, for example), cloning additional copies of a sequence onto a nucleic acid reagent, or altering an ORI to increase the number of copies of an epigenetic nucleic acid reagent. Non-limiting examples of alterations that can decrease the number of copies of a sequence of interest include, removing copies of the sequence of interest by deletion or disruption of regions in the genome, removing additional copies of the sequence from epigenetic nucleic acid reagents, or altering an ORI to decrease the number of copies of an epigenetic nucleic acid reagent.

In certain embodiments, increasing or decreasing the expression of a nucleotide sequence of interest can also be accomplished by altering, adding, or removing sequences involved in the expression of an anti-sense RNA, RNAi, siRNA, ribozyme, and the like. The methods described above can be used to modify expression of anti-sense RNA, RNAi, siRNA, ribozyme and the like.

Engineered microorganisms can be prepared by altering, introducing, or removing nucleotide sequences in the host genome or in stably maintained epigenetic nucleic acid reagents, as noted above. The nucleic acid reagents used to alter, introduce, or remove nucleotide sequences in the host genome or epigenetic nucleic acids can be prepared using the methods described herein or available to the artisan.

Nucleic acid sequences having a desired activity can be isolated from cells of a suitable organism using lysis and nucleic acid purification procedures available in Maniatis, T., E. F. Fritsch and J. Sambrook (1982) Molecular Cloning: a Laboratory Manual; Cold Spring Harbor Laboratory, Cold Spring Harbor, N.Y. or with commercially available cell lysis and DNA purification reagents and kits. In some embodiments, nucleic acids used to engineer microorganisms can be provided for conducting methods described herein after processing of the organism containing the nucleic acid. For example, the nucleic acid of interest may be extracted, isolated, purified or amplified from a sample (e.g., from an organism of interest or culture containing a plurality of organisms of interest, like yeast or bacteria for example). The term “isolated” as used herein refers to nucleic acid removed from its original environment (e.g., the natural environment if it is naturally occurring, or a host cell if expressed exogenously), and thus is altered “by the hand of man” from its original environment. An isolated nucleic acid generally is provided with fewer non-nucleic acid components (e.g., protein, lipid) than the amount of components present in a source sample. A composition comprising isolated sample nucleic acid can be substantially isolated (e.g., about 90%, 91%, 92%, 93%, 94%, 95%, 96%, 97%, 98%, 99% or greater than 99% free of non-nucleic acid components). The term “purified” as used herein refers to sample nucleic acid provided that contains fewer nucleic acid species than in the sample source from which the sample nucleic acid is derived. A composition comprising sample nucleic acid may be substantially purified (e.g., about 90%, 91%, 92%, 93%, 94%, 95%, 96%, 97%, 98%, 99% or greater than 99% free of other nucleic acid species). The term “amplified” as used herein refers to subjecting nucleic acid of a cell, organism or sample to a process that linearly or exponentially generates amplicon nucleic acids having the same or substantially the same nucleotide sequence as the nucleotide sequence of the nucleic acid in the sample, or portion thereof. As noted above, the nucleic acids used to prepare nucleic acid reagents as described herein can be subjected to fragmentation or cleavage.

Amplification of nucleic acids is sometimes necessary when dealing with organisms that are difficult to culture. Where amplification may be desired, any suitable amplification technique can be utilized. Non-limiting examples of methods for amplification of polynucleotides include polymerase chain reaction (PCR); ligation amplification (or ligase chain reaction (LCR)); amplification methods based on the use of Q-beta replicase or template-dependent polymerase (see US Patent Publication Number US20050287592); helicase-dependent isothermal amplification (Vincent et al., “Helicase-dependent isothermal DNA amplification”. EMBO reports 5 (8): 795-800 (2004)); strand displacement amplification (SDA); thermophilic SDA nucleic acid sequence based amplification (3SR or NASBA) and transcription-associated amplification (TAA). Non-limiting examples of PCR amplification methods include standard PCR, AFLP-PCR, Allele-specific PCR, Alu-PCR, Asymmetric PCR, Colony PCR, Hot start PCR, Inverse PCR (IPCR), In situ PCR (ISH), Intersequence-specific PCR (ISSR-PCR), Long PCR, Multiplex PCR, Nested PCR, Quantitative PCR, Reverse Transcriptase PCR (RT-PCR), Real Time PCR, Single cell PCR, Solid phase PCR, combinations thereof, and the like. Reagents and hardware for conducting PCR are commercially available.

Protocols for conducting the various type of PCR listed above are readily available to the artisan. PCR conditions can be dependent upon primer sequences, target abundance, and the desired amount of amplification, and therefore, one of skill in the art may choose from a number of PCR protocols available (see, e.g., U.S. Pat. Nos. 4,683,195 and 4,683,202; and PCR Protocols: A Guide to Methods and Applications, Innis et al., eds, 1990. PCR often is carried out as an automated process with a thermostable enzyme. In this process, the temperature of the reaction mixture is cycled through a denaturing region, a primer-annealing region, and an extension reaction region automatically. Machines specifically adapted for this purpose are commercially available. A non-limiting example of a PCR protocol that may be suitable for embodiments described herein is, treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Additional PCR protocols are described in the example section. Multiple cycles frequently are performed using a commercially available thermal cycler. Suitable isothermal amplification processes known and selected by the person of ordinary skill in the art also may be applied, in certain embodiments. In some embodiments, nucleic acids encoding polypeptides with a desired activity can be isolated by amplifying the desired sequence from an organism having the desired activity using oligonucleotides or primers designed based on sequences described herein.

Amplified, isolated and/or purified nucleic acids can be cloned into the recombinant DNA vectors described in Figures herein or into suitable commercially available recombinant DNA vectors. Cloning of nucleic acid sequences of interest into recombinant DNA vectors can facilitate further manipulations of the nucleic acids for preparation of nucleic acid reagents, (e.g., alteration of nucleotide sequences by mutagenesis, homologous recombination, amplification, and the like, for example). Standard cloning procedures (e.g., enzymic digestion, ligation, and the like) are readily available to the artisan and can be found in Maniatis, T., E. F. Fritsch and J. Sambrook (1982) Molecular Cloning: a Laboratory Manual; Cold Spring Harbor Laboratory, Cold Spring Harbor, N.Y.

In some embodiments, nucleic acid sequences prepared by isolation or amplification can be used, without any further modification, to add an activity to a microorganism and thereby generate a genetically modified or engineered microorganism. In certain embodiments, nucleic acid sequences prepared by isolation or amplification can be genetically modified to alter (e.g., increase or decrease, for example) a desired activity. In some embodiments, nucleic acids, used to add an activity to an organism, sometimes are genetically modified to optimize the heterologous polynucleotide sequence encoding the desired activity (e.g., polypeptide or protein, for example). The term “optimize” as used herein can refer to alteration to increase or enhance expression by preferred codon usage. The term optimize can also refer to modifications to the amino acid sequence to increase the activity of a polypeptide or protein, such that the activity exhibits a higher catalytic activity as compared to the “natural” version of the polypeptide or protein.

Nucleic acid sequences of interest can be genetically modified using methods known in the art. Mutagenesis techniques are particularly useful for small scale (e.g., 1, 2, 5, 10 or more nucleotides) or large scale (e.g., 50, 100, 150, 200, 500, or more nucleotides) genetic modification. Mutagenesis allows the artisan to alter the genetic information of an organism in a stable manner, either naturally (e.g., isolation using selection and screening) or experimentally by the use of chemicals, radiation, or inaccurate DNA replication (e.g., PCR mutagenesis). In some embodiments, genetic modification can be performed by whole scale synthetic synthesis of nucleic acids, using a native nucleotide sequence as the reference sequence, and modifying nucleotides that can result in the desired alteration of activity. Mutagenesis methods sometimes are specific or targeted to specific regions or nucleotides (e.g., site-directed mutagenesis, PCR-based site-directed mutagenesis, and in vitro mutagenesis techniques such as transplacement and in vivo oligonucleotide site-directed mutagenesis, for example). Mutagenesis methods sometimes are non-specific or random with respect to the placement of genetic modifications (e.g., chemical mutagenesis, insertion element (e.g., insertion or transposon elements) and inaccurate PCR based methods, for example).

Site directed mutagenesis is a procedure in which a specific nucleotide or specific nucleotides in a DNA molecule are mutated or altered. Site directed mutagenesis typically is performed using a nucleic acid sequence of interest cloned into a circular plasmid vector. Site-directed mutagenesis requires that the wild type sequence be known and used as a platform for the genetic alteration. Site-directed mutagenesis sometimes is referred to as oligonucleotide-directed mutagenesis because the technique can be performed using oligonucleotides which have the desired genetic modification incorporated into the complement a nucleotide sequence of interest. The wild type sequence and the altered nucleotide are allowed to hybridize and the hybridized nucleic acids are extended and replicated using a DNA polymerase. The double stranded nucleic acids are introduced into a host (e.g., E. coli, for example) and further rounds of replication are carried out in vivo. The transformed cells carrying the mutated nucleic acid sequence are then selected and/or screened for those cells carrying the correctly mutagenized sequence. Cassette mutagenesis and PCR-based site-directed mutagenesis are further modifications of the site-directed mutagenesis technique. Site-directed mutagenesis can also be performed in vivo (e.g., transplacement “pop-in pop-out”, In vivo site-directed mutagenesis with synthetic oligonucleotides and the like, for example).

PCR-based mutagenesis can be performed using PCR with oligonucleotide primers that contain the desired mutation or mutations. The technique functions in a manner similar to standard site-directed mutagenesis, with the exception that a thermocycler and PCR conditions are used to replace replication and selection of the clones in a microorganism host. As PCR-based mutagenesis also uses a circular plasmid vector, the amplified fragment (e.g., linear nucleic acid molecule) containing the incorporated genetic modifications can be separated from the plasmid containing the template sequence after a sufficient number of rounds of thermocycler amplification, using standard electrophoretic procedures. A modification of this method uses linear amplification methods and a pair of mutagenic primers that amplify the entire plasmid. The procedure takes advantage of the E. coli Dam methylase system which causes DNA replicated in vivo to be sensitive to the restriction endonucleases DpnI. PCR synthesized DNA is not methylated and is therefore resistant to DpnI. This approach allows the template plasmid to be digested, leaving the genetically modified, PCR synthesized plasmids to be isolated and transformed into a host bacteria for DNA repair and replication, thereby facilitating subsequent cloning and identification steps. A certain amount of randomness can be added to PCR-based sited directed mutagenesis by using partially degenerate primers.

Recombination sometimes can be used as a tool for mutagenesis. Homologous recombination allows the artisan to specifically target regions of known sequence for insertion of heterologous nucleotide sequences using the host organisms natural DNA replication and repair enzymes. Homologous recombination methods sometimes are referred to as “pop in pop out” mutagenesis, transplacement, knock out mutagenesis or knock in mutagenesis. Integration of a nucleic acid sequence into a host genome is a single cross over event, which inserts the entire nucleic acid reagent (e.g., pop in). A second cross over event excises all but a portion of the nucleic acid reagent, leaving behind a heterologous sequence, often referred to as a “footprint” (e.g., pop out). Mutagenesis by insertion (e.g., knock in) or by double recombination leaving behind a disrupting heterologous nucleic acid (e.g., knock out) both serve to disrupt or “knock out” the function of the gene or nucleic acid sequence in which insertion occurs. By combining selectable markers and/or auxotrophic markers with nucleic acid reagents designed to provide the appropriate nucleic acid target sequences, the artisan can target a selectable nucleic acid reagent to a specific region, and then select for recombination events that “pop out” a portion of the inserted (e.g., “pop in”) nucleic acid reagent.

Such methods take advantage of nucleic acid reagents that have been specifically designed with known target nucleic acid sequences at or near a nucleic acid or genomic region of interest. Popping out typically leaves a “foot print” of left over sequences that remain after the recombination event. The left over sequence can disrupt a gene and thereby reduce or eliminate expression of that gene. In some embodiments, the method can be used to insert sequences, upstream or downstream of genes that can result in an enhancement or reduction in expression of the gene. In certain embodiments, new genes can be introduced into the genome of a host organism using similar recombination or “pop in” methods. An example of a yeast recombination system using the ura3 gene and 5-FOA were described briefly above and further detail is presented herein.

A method for modification is described in Alani et al., “A method for gene disruption that allows repeated use of URA3 selection in the construction of multiply disrupted yeast strains”, Genetics 116(4):541-545 August 1987. The original method uses a Ura3 cassette with 1000 base pairs (bp) of the same nucleotide sequence cloned in the same orientation on either side of the URA3 cassette. Targeting sequences of about 50 bp are added to each side of the construct. The double stranded targeting sequences are complementary to sequences in the genome of the host organism. The targeting sequences allow site-specific recombination in a region of interest. The modification of the original technique replaces the two 1000 bp sequence direct repeats with two 200 bp direct repeats. The modified method also uses 50 bp targeting sequences. The modification reduces or eliminates recombination of a second knock out into the 1000 bp repeat left behind in a first mutagenesis, therefore allowing multiply knocked out yeast. Additionally, the 200 bp sequences used herein are uniquely designed, self-assembling sequences that leave behind identifiable footprints. The technique used to design the sequences incorporate design features such as low identity to the yeast genome, and low identity to each other. Therefore a library of the self-assembling sequences can be generated to allow multiple knockouts in the same organism, while reducing or eliminating the potential for integration into a previous knockout.

In yeast the cassettes are typically of two different overall structures, as follows:
<5′ homology region—promoter—coding sequence—terminator—3′ homology region>
<5′ homology region—promoter—coding sequence—terminator—marker—3′ homology region>

The parts of the DNA transformation cassette possess the following properties:

5′ and 3′ homology regions—these DNA sequences dictate the specific location in the chromosome where the DNA transformation cassette will insert through homologous recombination. The 5′ homology region indicates the upstream boundary for insertion while the 3′ homology indicates the downstream boundary. The homology regions may constitute a gene that rescues auxotrophy in an engineered microorganism, i.e. the homology regions mediate insertion into a non-functional gene that results in an auxotrophic phenotype and insertion of the cassette restores function to the gene. For example, the 5′ and 3′ homology regions may represent the two halves of the URA3 gene and direct homologous recombination in a mutant or loss-of-function ura3 gene, which rescues the loss-of-function.

Promoter—this DNA sequence drives transcription of the coding sequence immediately downstream of the promoter. A promoter will often be turned on or off when the microorganism is exposed to specific compounds or grown under certain conditions.

Coding sequence—the sequence of codons that are translated into the desired protein. The codons can be optimized to reflect the endogenous codon frequency of the engineered microorganism.

Terminator—this DNA sequence marks the end of the sequence to be transcribed, as indicated by the promoter. It may or may not contain sequences that positively or negatively regulate the activity of the promoter.

Marker—this DNA sequence encodes information that will confer properties to a yeast cell that mediate growth under selective or auxotrophic conditions. For example, if the initial cell line is auxotrophic for uracil and is transformed with a cassette containing a URA3 marker, any transformant that contains the URA3 marker will now be able to grow in the absence of uracil.

The DNA transformation cassette is generated through conventional molecular biology methods such as PCR, restriction enzyme digestion and DNA ligation and/or Gibson assembly. The cassette is transformed into the yeast and clones of interest are identified as colonies that grow on the appropriate selective or auxotrophic media, e.g. synthetic complete yeast media lacking uracil.

As noted above, the URA3 cassette makes use of the toxicity of 5-FOA in yeast carrying a functional URA3 gene. Uracil synthesis deficient yeast are transformed with the modified URA3 cassette, using standard yeast transformation protocols, and the transformed cells are plated on minimal media minus uracil. In some embodiments, PCR can be used to verify correct insertion into the region of interest in the host genome, and certain embodiments the PCR step can be omitted. Inclusion of the PCR step can reduce the number of transformants that need to be counter selected to “pop out” the URA3 cassette. The transformants (e.g., all or the ones determined to be correct by PCR, for example) can then be counter-selected on media containing 5-FOA, which will select for recombination out (e.g., popping out) of the URA3 cassette, thus rendering the yeast ura3 deficient again, and resistant to 5-FOA toxicity. Targeting sequences used to direct recombination events to specific regions are presented herein. A modification of the method described above can be used to integrate genes into the chromosome, where after recombination a functional gene is left in the chromosome next to the 200 bp footprint.

In some embodiments, other auxotrophic or dominant selection markers can be used in place of URA3 (e.g., an auxotrophic selectable marker), with the appropriate change in selection media and selection agents. Auxotrophic selectable markers are used in strains deficient for synthesis of a required biological molecule (e.g., amino acid or nucleoside, for example). Non-limiting examples of additional auxotrophic markers include; HIS3, TRP1, LEU2, LEU2-d, and LYS2. Certain auxotrophic markers (e.g., URA3 and LYS2) allow counter selection to select for the second recombination event that pops out all but one of the direct repeats of the recombination construct. HIS3 encodes an activity involved in histidine synthesis. TRP1 encodes an activity involved in tryptophan synthesis. LEU2 encodes an activity involved in leucine synthesis. LEU2-d is a low expression version of LEU2 that selects for increased copy number (e.g., gene or plasmid copy number, for example) to allow survival on minimal media without leucine. LYS2 encodes an activity involved in lysine synthesis and allows counter selection for recombination out of the LYS2 gene using alpha-amino adipate (α-amino adipate).

Dominant selectable markers are useful because they also allow industrial and/or prototrophic strains to be used for genetic manipulations. Additionally, dominant selectable markers provide the advantage that rich medium can be used for plating and culture growth, and thus growth rates are markedly increased. Non-limiting examples of dominant selectable markers include; Tn903 kan^{r}, Cm^{r}, Hyg^{r}, CUP1, and DHFR. Tn903 kan^{r} encodes an activity involved in kanamycin antibiotic resistance (e.g., typically neomycin phosphotransferase II or NPTII, for example). Cm^{r} encodes an activity involved in chloramphenicol antibiotic resistance (e.g., typically chloramphenicol acetyl transferase or CAT, for example). Hyg^{r} encodes an activity involved in hygromycin resistance by phosphorylation of hygromycin B (e.g., hygromycin phosphotransferase, or HPT). CUP1 encodes an activity involved in resistance to heavy metal (e.g., copper, for example) toxicity. DHFR encodes a dihydrofolate reductase activity which confers resistance to methotrexate and sulfanilamide compounds.

In contrast to site-directed or specific mutagenesis, random mutagenesis does not require any sequence information and can be accomplished by a number of widely different methods. Random mutagenesis often is used to generate mutant libraries that can be used to screen for the desired genotype or phenotype. Non-limiting examples of random mutagenesis include; chemical mutagenesis, UV-induced mutagenesis, insertion element or transposon-mediated mutagenesis, DNA shuffling, error-prone PCR mutagenesis, and the like.

Chemical mutagenesis often involves chemicals like ethyl methanesulfonate (EMS), nitrous acid, mitomycin C, N-methyl-N-nitrosourea (MNU), diepoxybutane (DEB), 1,2,7,8-diepoxyoctane (DEO), methyl methane sulfonate (MMS), N-methyl-N′-nitro-N-nitrosoguanidine (MNNG), 4-nitroquinoline 1-oxide (4-NQO), 2-methyloxy-6-chloro-9(3-[ethyl-2-chloroethyl]-aminopropylamino)-acridinedihydrochloride (ICR-170), 2-amino purine (2AP), and hydroxylamine (HA), provided herein as non-limiting examples. These chemicals can cause base-pair substitutions, frameshift mutations, deletions, transversion mutations, transition mutations, incorrect replication, and the like. In some embodiments, the mutagenesis can be carried out in vivo. Sometimes the mutagenic process involves the use of the host organism's DNA replication and repair mechanisms to incorporate and replicate the mutagenized base or bases.

Another type of chemical mutagenesis involves the use of base-analogs. The use of base-analogs causes incorrect base pairing which in the following round of replication is corrected to a mismatched nucleotide when compared to the starting sequence. Base analog mutagenesis introduces a small amount of non-randomness to random mutagenesis, because specific base analogs can be chosen which can be incorporated at certain nucleotides in the starting sequence. Correction of the mispairing typically yields a known substitution. For example, Bromo-deoxyuridine (BrdU) can be incorporated into DNA and replaces T in the sequence. The host DNA repair and replication machinery can sometime correct the defect, but sometimes will mispair the BrdU with a G. The next round of replication then causes a G-C transversion from the original A-T in the native sequence.

Ultraviolet (UV) induced mutagenesis is caused by the formation of thymidine dimers when UV light irradiates chemical bonds between two adjacent thymine residues. Excision repair mechanism of the host organism corrects the lesion in the DNA, but occasionally the lesion is incorrectly repaired typically resulting in a C to T transition.

Insertion element or transposon-mediated mutagenesis makes use of naturally occurring or modified naturally occurring mobile genetic elements. Transposons often encode accessory activities in addition to the activities necessary for transposition (e.g., movement using a transposase activity, for example). In many examples, transposon accessory activities are antibiotic resistance markers (e.g., see Tn903 kan^{r} described above, for example). Insertion elements typically only encode the activities necessary for movement of the nucleic acid sequence. Insertion element and transposon mediated mutagenesis often can occur randomly, however specific target sequences are known for some transposons. Mobile genetic elements like IS elements or Transposons (Tn) often have inverted repeats, direct repeats or both inverted and direct repeats flanking the region coding for the transposition genes. Recombination events catalyzed by the transposase cause the element to remove itself from the genome and move to a new location, leaving behind a portion of an inverted or direct repeat. Classic examples of transposons are the “mobile genetic elements” discovered in maize. Transposon mutagenesis kits are commercially available which are designed to leave behind a 5 codon insert (e.g., Mutation Generation System kit, Finnzymes, World Wide Web URL finnzymes.us, for example). This allows the artisan to identify the insertion site, without fully disrupting the function of most genes.

DNA shuffling is a method which uses DNA fragments from members of a mutant library and reshuffles the fragments randomly to generate new mutant sequence combinations. The fragments are typically generated using DNaseI, followed by random annealing and re-joining using self-priming PCR. The DNA overhanging ends, from annealing of random fragments, provide “primer” sequences for the PCR process. Shuffling can be applied to libraries generated by any of the above mutagenesis methods.

Error prone PCR and its derivative rolling circle error prone PCR uses increased magnesium and manganese concentrations in conjunction with limiting amounts of one or two nucleotides to reduce the fidelity of the Taq polymerase. The error rate can be as high as 2% under appropriate conditions, when the resultant mutant sequence is compared to the wild type starting sequence. After amplification, the library of mutant coding sequences must be cloned into a suitable plasmid. Although point mutations are the most common types of mutation in error prone PCR, deletions and frameshift mutations are also possible. There are a number of commercial error-prone PCR kits available, including those from Stratagene and Clontech (e.g., World Wide Web URL strategene.com and World Wide Web URL clontech.com, respectively, for example). Rolling circle error-prone PCR is a variant of error-prone PCR in which wild-type sequence is first cloned into a plasmid, the whole plasmid is then amplified under error-prone conditions.

As noted above, organisms with altered activities can also be isolated using genetic selection and screening of organisms challenged on selective media or by identifying naturally occurring variants from unique environments. For example, 2-Deoxy-D-glucose is a toxic glucose analog. Growth of yeast on this substance yields mutants that are glucose-deregulated. A number of mutants have been isolated using 2-Deoxy-D-glucose including transport mutants, and mutants that ferment glucose and galactose simultaneously instead of glucose first then galactose when glucose is depleted. Similar techniques have been used to isolate mutant microorganisms that can metabolize plastics (e.g., from landfills), petrochemicals (e.g., from oil spills), and the like, either in a laboratory setting or from unique environments.

Similar methods can be used to isolate naturally occurring mutations in a desired activity when the activity exists at a relatively low or nearly undetectable level in the organism of choice, in some embodiments. The method generally consists of growing the organism to a specific density in liquid culture, concentrating the cells, and plating the cells on various concentrations of the substance to which an increase in metabolic activity is desired. The cells are incubated at a moderate growth temperature, for 5 to 10 days. To enhance the selection process, the plates can be stored for another 5 to 10 days at a low temperature. The low temperature sometimes can allow strains that have gained or increased an activity to continue growing while other strains are inhibited for growth at the low temperature. Following the initial selection and secondary growth at low temperature, the plates can be replica plated on higher or lower concentrations of the selection substance to further select for the desired activity.

A native, heterologous, or mutagenized polynucleotide can be introduced into a nucleic acid reagent for introduction into a host organism, thereby generating an engineered microorganism. Standard recombinant DNA techniques (restriction enzyme digests, ligation, and the like) can be used by the artisan to combine the mutagenized nucleic acid of interest into a suitable nucleic acid reagent capable of (i) being stably maintained by selection in the host organism, or (ii) being integrating into the genome of the host organism. As noted above, sometimes nucleic acid reagents comprise two replication origins to allow the same nucleic acid reagent to be manipulated in bacterial before final introduction of the final product into the host organism (e.g., yeast or fungus for example). Standard molecular biology and recombinant DNA methods available to one of skill in the art can be found in Maniatis, T., E. F. Fritsch and J. Sambrook (1982) Molecular Cloning: a Laboratory Manual; Cold Spring Harbor Laboratory, Cold Spring Harbor, N.Y.

Nucleic acid reagents can be introduced into microorganisms using various techniques. Non-limiting examples of methods used to introduce heterologous nucleic acids into various organisms include; transformation, transfection, transduction, electroporation, ultrasound-mediated transformation, particle bombardment and the like. In some instances, the addition of carrier molecules (e.g., bis-benzimdazolyl compounds, for example, see U.S. Pat. No. 5,595,899) can increase the uptake of DNA in cells typically though to be difficult to transform by conventional methods. Conventional methods of transformation are readily available to the artisan and can be found in Maniatis, T., E. F. Fritsch and J. Sambrook (1982) Molecular Cloning: a Laboratory Manual; Cold Spring Harbor Laboratory, Cold Spring Harbor, N.Y.

Usually a DNA piece is constructed (called cassette) for the integration of the gene of interest into the genome.

### Culture, Production and Process Methods

Engineered microorganisms often are cultured under conditions that optimize yield of a target molecule. A non-limiting example of such a target molecule is ethanol. Culture conditions often can alter (e.g., add, optimize, reduce, or eliminate, for example) activity of one or more of the following activities: phosphofructokinase activity, phosphogluconate dehydratase activity, 2-keto-3-deoxygluconate-6-phosphate aldolase activity, xylose isomerase activity, phosphoenolpyruvate carboxylase activity, alcohol dehydrogenase 2 activity and thymidylate synthase activities. In general, conditions that may be optimized include the type and amount of carbon source, the type and amount of nitrogen source, the carbon-to-nitrogen ratio, the oxygen level, growth temperature, pH, length of the biomass production phase, length of target product accumulation phase, and time of cell harvest.

The term “fermentation conditions” as used herein refers to any culture conditions suitable for maintaining a microorganism (e.g., in a static or proliferative state). Fermentation conditions can include several parameters, including without limitation, temperature, oxygen content, nutrient content (e.g., glucose content), pH, agitation level (e.g., revolutions per minute), gas flow rate (e.g., air, oxygen, nitrogen gas), redox potential, cell density (e.g., optical density), cell viability and the like. A change in fermentation conditions (e.g., switching fermentation conditions) is an alteration, modification or shift of one or more fermentation parameters. For example, one can change fermentation conditions by increasing or decreasing temperature, increasing or decreasing pH (e.g., adding or removing an acid, a base or carbon dioxide), increasing or decreasing oxygen content (e.g., introducing air, oxygen, carbon dioxide, nitrogen) and/or adding or removing a nutrient (e.g., one or more sugars or sources of sugar, biomass, vitamin and the like), or combinations of the foregoing. Examples of fermentation conditions are described herein. Aerobic conditions often comprise greater than about 50% dissolved oxygen (e.g., about 52%, 54%, 56%, 58%, 60%, 62%, 64%, 66%, 68%, 70%, 72%, 74%, 76%, 78%, 80%, 82%, 84%, 86%, 88%, 90%, 91%, 92%, 93%, 94%, 95%, 96%, 97%, 98% or 99%, or greater than any one of the foregoing). Anaerobic conditions often comprise less than about 50% dissolved oxygen (e.g., about 1%, 2%, 4%, 6%, 8%, 10%, 12%, 14%, 16%, 18%, 20%, 22%, 24%, 26%, 28%, 30%, 32%, 34%, 36%, 38%, 40%, 42%, 44%, 46%, 48%, or less than any one of the foregoing).

Culture media generally contain a suitable carbon source. Carbon sources may include, but are not limited to, monosaccharides (e.g., glucose, fructose, xylose), disaccharides (e.g., lactose, sucrose), oligosaccharides, polysaccharides (e.g., starch, cellulose, hemicellulose, other lignocellulosic materials, or mixtures thereof), sugar alcohols (e.g., glycerol), and renewable feedstocks (e.g., cheese whey permeate, corn steep liquor, sugar beet molasses, barley malt). Carbon sources also can be selected from one or more of the following non-limiting examples: linear or branched alkanes (e.g., hexane), linear or branched alcohols (e.g., hexanol), fatty acids (e.g., about 10 carbons to about 22 carbons), esters of fatty acids, monoglycerides, diglycerides, triglycerides, phospholipids, and various commercial sources of fatty acids including vegetable oils (e.g., soybean oil) and animal fats. A carbon source may include one-carbon sources (e.g., carbon dioxide, methanol, formaldehyde, formate, and carbon-containing amines) from which metabolic conversion into key biochemical intermediates can occur. It is expected that the source of carbon utilized may encompass a wide variety of carbon-containing sources and will only be limited by the choice of the engineered microorganism(s).

Nitrogen may be supplied from an inorganic (e.g., (NH.sub.4).sub.2SO.sub.4) or organic source (e.g., urea or glutamate). In addition to appropriate carbon and nitrogen sources, culture media also can contain suitable minerals, salts, cofactors, buffers, vitamins, metal ions (e.g., Mn.sup.+2, Co.sup.+2, Zn.sup.+2, Mg.sup.+2) and other components suitable for culture of microorganisms. Engineered microorganisms sometimes are cultured in complex media (e.g., yeast extract-peptone-dextrose broth (YPD)). In some embodiments, engineered microorganisms are cultured in a defined minimal media that lacks a component necessary for growth and thereby forces selection of a desired expression cassette (e.g., Yeast Nitrogen Base (DIFCO Laboratories, Detroit, Mich.)). Culture media in some embodiments are common commercially prepared media, such as Yeast Nitrogen Base (DIFCO Laboratories, Detroit, Mich.). Other defined or synthetic growth media may also be used and the appropriate medium for growth of the particular microorganism are known.

A variety of host organisms can be selected for the production of engineered microorganisms. Non-limiting examples include yeast and fungi. In specific embodiments, yeast are cultured in YPD media (10 g/L Bacto Yeast Extract, 20 g/L Bacto Peptone, and 20 g/L Dextrose). Filamentous fungi, in particular embodiments, are grown in CM (Complete Medium) containing 10 g/L Dextrose, 2 g/L Bacto Peptone, 1 g/L Bacto Yeast Extract, 1 g/L Casamino acids, 50 mL/L 20× Nitrate Salts (120 g/L NaNO₃, 10.4 g/L KCl, 10.4 g/L MgSO₄·7H₂O), 1 mL/L 1000× Trace Elements (22 g/L ZnSO₄·7H₂O, 11 g/L H₃BO₃, 5 g/L MnCl₂·7 H₂O, 5 g/L FeSO₄·7H₂O, 1.7 g/L CoCl₂·6H₂O, 1.6 g/L CuSO₄·5H₂O, 1.5 g/L Na₂MoO₄·2H₂O, and 50 g/L Na₄EDTA), and 1 mL/L Vitamin Solution (100 mg each of Biotin, pyridoxine, thiamine, riboflavin, p-aminobenzoic acid, and nicotinic acid in 100 mL water).

A suitable pH range for the fermentation often is between about pH 4.0 to about pH 8.0, where a pH in the range of about pH 5.5 to about pH 7.0 sometimes is utilized for initial culture conditions. Culturing may be conducted under aerobic or anaerobic conditions, where microaerobic conditions sometimes are maintained. A two-stage process may be utilized, where one stage promotes microorganism proliferation, and another state promotes production of target molecule. In a two-stage process, the first stage may be conducted under aerobic conditions (e.g., introduction of air and/or oxygen) and the second stage may be conducted under anaerobic conditions (e.g., air or oxygen are not introduced to the culture conditions).

A variety of fermentation processes may be applied for commercial biological production of a target product. In some embodiments, commercial production of a target product from a recombinant microbial host is conducted using a batch, fed batch or continuous fermentation process, for example.

A batch fermentation process often is a closed system where the media composition is fixed at the beginning of the process and not subject to further additions beyond those required for maintenance of pH and oxygen level during the process. At the beginning of the culturing process the media is inoculated with the desired organism and growth or metabolic activity is permitted to occur without adding additional sources (i.e., carbon and nitrogen sources) to the medium. In batch processes the metabolite and biomass compositions of the system change constantly up to the time the culture is terminated. In a typical batch process, cells proceed through a static lag phase to a high-growth log phase and finally to a stationary phase, wherein the growth rate is diminished or halted. Left untreated, cells in the stationary phase will eventually die.

A variation of the standard batch process is the fed-batch process, where the carbon source is continually added to the fermentor over the course of the fermentation process. Fed-batch processes are useful when catabolite repression is apt to inhibit the metabolism of the cells or where it is desirable to have limited amounts of carbon source in the media at any one time. Measurement of the carbon source concentration in fed-batch systems may be estimated on the basis of the changes of measurable factors such as pH, dissolved oxygen, and the partial pressure of waste gases (e.g., CO.sub.2). Batch and fed-batch culturing methods are known in the art. Examples of such methods may be found in Thomas D. Brock in Biotechnology: A Textbook of Industrial Microbiology, 2.sup.nd ed., (1989) Sinauer Associates Sunderland, Mass. and Deshpande, Mukund V., Appl. Biochem. Biotechnol., 36:227 (1992).

In continuous fermentation process a defined media often is continuously added to a bioreactor while an equal amount of culture volume is removed simultaneously for product recovery. Continuous cultures generally maintain cells in the log phase of growth at a constant cell density. Continuous or semi-continuous culture methods permit the modulation of one factor or any number of factors that affect cell growth or end product concentration. For example, an approach may limit the carbon source and allow all other parameters to moderate metabolism. In some systems, a number of factors affecting growth may be altered continuously while the cell concentration, measured by media turbidity, is kept constant. Continuous systems often maintain steady state growth and thus the cell growth rate often is balanced against cell loss due to media being drawn off the culture. Methods of modulating nutrients and growth factors for continuous culture processes, as well as techniques for maximizing the rate of product formation, are known and a variety of methods are detailed by Brock, supra.

In various embodiments ethanol may be purified from the culture media or extracted from the engineered microorganisms. Culture media may be tested for ethanol concentration and drawn off when the concentration reaches a predetermined level. Detection methods are known in the art, including but not limited to the use of a hydrometer and infrared measurement of vibrational frequency of dissolved ethanol using the CH band at 2900 cm⁻¹. Ethanol may be present at a range of levels as described herein.

A target product sometimes is retained within an engineered microorganism after a culture process is completed, and in certain embodiments, the target product is secreted out of the microorganism into the culture medium. For the latter embodiments, (i) culture media may be drawn from the culture system and fresh medium may be supplemented, and/or (ii) target product may be extracted from the culture media during or after the culture process is completed. Engineered microorganisms may be cultured on or in solid, semi-solid or liquid media. In some embodiments media is drained from cells adhering to a plate. In certain embodiments, a liquid-cell mixture is centrifuged at a speed sufficient to pellet the cells but not disrupt the cells and allow extraction of the media, as known in the art. The cells may then be resuspended in fresh media. Target product may be purified from culture media according to methods known in the art.

In certain embodiments, target product is extracted from the cultured engineered microorganisms. The microorganism cells may be concentrated through centrifugation at a speed sufficient to shear the cell membranes. In some embodiments, the cells may be physically disrupted (e.g., shear force, sonication) or chemically disrupted (e.g., contacted with detergent or other lysing agent).

The phases may be separated by centrifugation or other method known in the art and target product may be isolated according to known methods.

Commercial grade target product sometimes is provided in substantially pure form (e.g., 90% pure or greater, 95% pure or greater, 99% pure or greater or 99.5% pure or greater). In some embodiments, target product may be modified into any one of a number of downstream products. For example, cannabidiolic acid may be derivatized or further processed to be an ingredient in food, drinks, vape pens, gum, skin lotions, pharmaceuticals, and supplements.

Target product may be provided within cultured microbes containing target product, and cultured microbes may be supplied fresh or frozen in a liquid media or dried. Fresh or frozen microbes may be contained in appropriate moisture-proof containers that may also be temperature controlled as necessary. Target product sometimes is provided in culture medium that is substantially cell-free. In some embodiments target product or modified target product purified from microbes is provided, and target product sometimes is provided in substantially pure form. In certain embodiments, ethanol can be provided in anhydrous or hydrous forms. Ethanol may be transported in a variety of containers including pints, quarts, liters, gallons, drums (e.g., 10 gallon or 55 gallon, for example) and the like.

### EXAMPLES

The examples set forth below illustrate certain embodiments and do not limit the technology. Certain examples set forth below utilize standard recombinant DNA and other biotechnology protocols known in the art. Many such techniques are described in detail in Maniatis, T., E. F. Fritsch and J. Sambrook (1982) Molecular Cloning: a Laboratory Manual; Cold Spring Harbor Laboratory, Cold Spring Harbor, N.Y. DNA mutagenesis can be accomplished using the Stratagene (San Diego, Calif) “QuickChange” kit according to the manufacturer's instructions, or by one of the other types of mutagenesis described above. 5,000 bp DNA pieces can also be manufactured to order by companies such as Twist Biosciences.

### Example 1—Construction of Plasmids for Candida viswanathii

Protein sequence was reverse translated into DNA sequence to reflect the use of yeast alternative genetic code and codon usage in Candida viswanathii, reduction of secondary structure, and absence of certain restriction enzyme recognition sites. Other DNA sequences that encode the same protein may also be used. The following sequences were used as open reading frames for genes used to genetically modify Candida viswanathii.

CvACO1P Gene
(SEQ ID NO: 93)
ATGACAGAAGTAGTAGATAGAGCAAGTTCCCCAGCAAGTCCAGGATCTACGACCGC
CGCCGCAGACGGTGCTAAGGTGGCGGTGGAGCCACGCGTAGATGTAGCGGCCCTTG
GCGAGCAGTTGCTAGGGCGATGGGCTGACATCAGATTGCACGCACGAGACTTAGCA
GGCCGCGAAGTGGTCCAAAAGGTTGAAGGACTTACGCACACTGAGCATCGGAGTAG
AGTCTTTGGACAGTTGAAGTACTTGGTAGACAACAATGCTGTTCACAGAGCTTTTCC
CTCCAGGCTAGGTGGATCAGATGACCATGGCGGTAATATAGCTGGATTCGAGGAAT
TAGTTACTGCTGATCCATCATTGCAAATAAAGGCCGGCGTTCAGTGGGGTTTGTTTG
GTTCTGCAGTGATGCACTTGGGAACCCGTGAACATCATGACAAGTGGTTGCCAGGTA
TTATGTCGTTAGAAATACCGGGGTGTTTCGCGATGACAGAAACCGGGCACGGTAGC
GACGTGGCCTCTATTGCTACAACAGCAACTTATGATGAGGAAACCCAAGAGTTTGTT
ATTGATACCCCGTTCAGAGCCGCTTGGAAAGATTATATCGGTAATGCAGCGAACGAT
GGTTTGGCGGCAGTTGTTTTCGCACAATTAATCACGAGGAAAGTGAACCATGGTGTA
CACGCCTTTTACGTGGATCTCAGAGATCCTGCGACTGGAGACTTCCTACCCGGAATA
GGAGGAGAGGACGATGGAATCAAGGGGGGATTGAATGGCATTGACAACGGTAGAC
TACATTTTACGAACGTACGCATTCCTAGAACTAATCTTCTTAACAGATATGGCGATG
TGGCGGTCGACGGCACATACCTGTCGACCATCGAATCACCAGGGCGCCGGTTCTTTA
CGATGCTTGGTACTCTAGTCCAGGGTAGAGTTAGTCTCGATGGAGCAGCTGTCGCTG
CACTGAAGGTCGCATTGCAAAGTGCAATTCACTACGCTGCGGAGAGGAGACAATTT
AATGCGACTTCACCTACTGAAGAAGAGGTCCTTCTTGATTATCAGAGGCATCAAAGG
AGACTCTTTACACGACTTGCAACGACGTACGCCGCATCTTTCGCCCACGAGCAGCTA
TTGCAAAAGTTCGATGATGTCTTTTCAGGGGCACATGATACCGACGCCGACCGGCAG
GACTTGGAAACCCTAGCCGCCGCTTTGAAGCCATTGAGCACATGGCATGCACTTGAC
ACGTTACAAGAATGCAGAGAGGCCTGTGGGGGGGCCGGATTTTTGATAGAAAACCG
TTTCGCGAGCTTGCGTGCTGACTTGGACGTTTACGTCACATTCGAGGGTGATAACAC
AGTTTTATTGCAATTGGTTGCTAAACGGCTCTTGGCAGACTACGCAAAAGAGTTCAG
AGGGGCCAACTTCGGCGTTCTTGCCAGGTATGTGGTTGACCAAGCCGCGGGAGTGG
CGCTCCACCGAACAGGACTAAGGCAAGTCGCTCAATTTGTTGCAGACAGCGGGTCC
GTTCAGAAGTCGGCTCTTGCGCTTCGCGATGAAGAGGGTCAACGAACATTGTTAACG
GACAGAGTACAGAGCATGGTTGCCGAAGTGGGGGCTGCCTTGAAAGGCGCAGGCAA
ATTACCCCAACATCAAGCAGCTGCATTGTTCAACCAACACCAGAACGAACTTATTGA
GGCTGCCCAGGCCCATGCAGAACTCCTCCAATGGGAGGCATTTACAGAAGCTCTCG
CTAAAGTCGACGATGCTGGTACAAAGGAAGTGCTTACTCGATTGCGAGATCTCTTTG
GTTTGTCCTTGATTGAAAAACACTTGCTGTGGTATCTTATGAATGGACGTTTGTCCAT
GCAAAGAGGCAGGACAGTTGGAACTTACATTAATCGTTTACTTGTCAAGATCCGTCC
ACACGCACTAGACTTGGTTGATGCCTTCGGTTACGGCGCGGAGCATTTGCGTGCTGC
TATCGCCACCGGAGCGGAAGCAACCCGACAGGATGAAGCCCGAACGTATTTTAGAC
AACAACGGGCATCGGGACTGGCCCCGGCCGATGAAAAGACCTTACTCGCTATCAAA
GCTGGTAAATCAAGAGGGCGAAGGGCAAAGCTATGA
CvHXS1 Gene
(SEQ ID NO: 94)
ATGGGAAAAAATTATAAATCTTTGGACTCAGTTGTGGCTAGTGACTTCATTGCACTT
GGGATCACATCAGAAGTTGCTGAGACATTGCACGGACGCTTGGCAGAGATAGTTTG
CAACTACGGCGCCGCAACACCTCAGACCTGGATTAACATCGCAAACCATATTCTAA
GTCCAGATCTTCCATTTAGTCTCCATCAGATGTTGTTCTACGGTTGTTATAAGGACTT
TGGTCCAGCACCCCCAGCTTGGATACCAGACCCCGAAAAAGTAAAGTCCACGAACT
TAGGTGCCTTGTTAGAAAAGCGGGGAAAGGAGTTTCTAGGCGTTAAGTATAAGGAC
CCAATAAGTCTGTTTTCTCACTTCCAGGAGTTTAGCGTTCGAAATCCGGAAGTCTACT
GGCGGACGGTACTTATGGATGAAATGAAGATACTGTTCAGCAAAGATCCCGAATGT
ATCCTCAGACGCGACGACATTAACAACCCAGGGGGCTCTGAGTGGCTACCAGGTGG
ATATCTCAACCTGGCCAAGAACTGTTTGAATGTAAATAGTAACAAAAAACTTAACG
ACACTATGATAGTGTGGAGAGATGAAGGAAATGACGATCTCCCATTGAATAAATTG
ACTCTTGATCAATTACGAAAACGAGTCTGGTTGGTTGGATACGCCCTAGAAGAGATG
GGCCTTGAGAAGGGATGTGCGATTGCAATTGACATGCCCATGCACGTAGATGCGGTT
GTGATCTATTTAGCTATCGTCTTGGCAGGCTACGTCGTTGTCTCCATTGCAGATTCAT
TCTCAGCACCGGAAATTTCCACAAGATTGCGTCTATCAAAGGCTAAGGCTATTTTTA
CACAAGATCATATCATCCGAGGGAAAAAGCGTATACCTTTGTACCTGCGTGTCGTCG
AGGCCAAGTCTCCGATGGCAATAGTTATCCCGTGTTCGGGTTCAAATATTGGTGCGG
AATTGCGGGATGGTGATATTCTGTGGGATTACTTCTTAGAACGCGCAAAGGAATTTA
AGAACTGCGAATTTACAGCCCGTGAACAGCCAGTGGACGCGTACACAAATATTTTGT
TCTCATCGGGAACCACCGGAGAGCCAAAGGCGATACCATGGACTCAAGCTACGCCT
CTCAAGGCGGCTGCTGATGGTTGGTCACACTTGGACATTAGAAAGGGTGACGTAATT
GTATGGCCTACCAATTTGGGGTGGATGATGGGGCCTTGGTTGGTCTATGCTTCACTC
CTTAACGGGGCAAGCATCGCATTGTATAACGGATCTCCACTAGTGTCCGGCTTTGCC
AAATTCGTTCAAGATGCGAAAGTTACTATGCTAGGAGTTGTCCCCTCCATCGTACGA
AGCTGGAAAAGCACTAATTGCGTTAGTGGGTACGATTGGTCTACAATCAGATGCTTC
TCCTCATCGGGTGAGGCATCGAATGTCGATGAATACTTATGGCTAATGGGAAGGGCT
AACTACAAACCGGTCATCGAAATGTGCGGTGGCACAGAGATCGGGGGTGCCTTCAG
CGCCGGTTCGTTTTTACAAGCCCAATCTTTGAGTAGCTTCTCATCCCAATGTATGGGA
TGCACCTTGTACATTCTCGACAAGAATGGCTACCCGATGCCAAAGAACAAGCCGGG
TATAGGTGAATTGGCCTTGGGACCCGTGATGTTCGGTGCTTCCAAGACTTTACTTAA
CGGAAACCATCATGACGTTTATTTCAAAGGCATGCCCACCTTGAACGGAGAAGTCTT
GAGGAGACACGGAGATATCTTCGAACTCACTTCGAACGGCTATTATCACGCTCATGG
TAGAGCAGATGACACGATGAATATCGGGGGGATTAAAATTTCCTCAATCGAGATTG
AAAGGGTGTGTAATGAAGTTGACGATAGAGTGTTTGAGACTACGGCCATTGGAGTG
CCTCCATTGGGCGGAGGTCCAGAGCAGCTCGTTATCTTTTTTGTTCTTAAGGACAGC
AATGATACGACCATCGACCTAAACCAATTGCGACTTAGTTTTAATCTTGGGTTACAA
AAGAAATTGAACCCACTTTTTAAGGTGACGAGGGTTGTGCCACTTTCGCTGTTGCCT
AGGACAGCCACCAACAAAATAATGAGAAGAGTGCTTAGACAGCAATTTAGTCATTT
CGAGTGA
CvTKS1 Gene
(SEQ ID NO: 95)
ATGAATCATTTAAGAGCAGAAGGACCCGCATCAGTGTTAGCGATAGGTACAGCTAA
CCCAGAGAATATCTTAATCCAAGATGAATTTCCTGACTACTATTTCCGTGTTACTAA
ATCGGAACATATGACTCAACTTAAAGAGAAGTTCCGGAAAATCTGCGATAAATCCA
TGATCCGAAAGAGAAACTGTTTCCTTAACGAAGAACATCTCAAGCAAAACCCGAGG
TTGGTAGAGCACGAAATGCAGACCTTGGATGCTAGGCAGGACATGTTGGTGGTCGA
AGTGCCAAAACTCGGCAAGGACGCGTGCGCTAAGGCAATCAAGGAGTGGGGTCAAC
CGAAGTCTAAAATCACGCATCTAATATTTACATCTGCACTGACAACCGACATGCCGG
GTGCCGATTATCACTGCGCCAAGCTACTTGGATTGAGTCCACTGGTTAAGAGAGTTA
TGATGTATCAATTGGGGTGTTACGGAGGGGGCACAGTCCTCAGAATTGCTAAGGAT
ATTGCGGAAAATAACAAGGGCGCGAGGGTCCTTGCTGTATGTTGTGATATTATGGCC
TGTTTGTTTCGCGGGCCCTCGGATTCAGATTTGGAATTGCTTGTCGGACAGGCAATTT
TTGGTGACGGGGCCGCAGCAGTCATAGTGGGAGCCGAACCAGACGAAAGCGTGGGT
GAAAGACCAATCTTTGAGTTGGTTCTGACCGGACAAACGATCTTACCTAACTCGGAA
GGTACGATTGGAGGACATATTAGAGAAGCCGGCCTAATTTTCGATCTTCACAAAGAC
GTTCCAATGTTAATCTCCAATAACATAGAAAAGTGCTTGATAGAAGCATTTACTCCC
ATTGGTATTAGTGACTGGAACAGCATTTTCTGGATCACCCACCCTGGAGGAAAAGCT
ATACTCGATAAGGTTGAAGAGAAACTCGACTTGAAAAAGGAGAAATTCGTTGACTC
ACGACATGTGTTATCAGAGCACGGGAATATGAGTTCATCCACAGTCTTGTTCGTAAT
GGATGAATTGCGAAAACGCTCTCTTGAGGAGGGAAAGAGCACAACCGGTGACGGGT
TTGAGTGGGGCGTGCTATTCGGTTTTGGCCCAGGTTTGACTGTCGAGCGGGTTGTTG
TTCGTAGTGTACCAATTAAGTACTGA
CvTKS1P Gene
(SEQ ID NO: 96)
ATGAATCATTTAAGAGCAGAAGGACCCGCATCAGTGTTAGCGATAGGTACAGCTAA
CCCAGAGAATATCTTAATCCAAGATGAATTTCCTGACTACTATTTCCGTGTTACTAA
ATCGGAACATATGACTCAACTTAAAGAGAAGTTCCGGAAAATCTGCGATAAATCCA
TGATCCGAAAGAGAAACTGTTTCCTTAACGAAGAACATCTCAAGCAAAACCCGAGG
TTGGTAGAGCACGAAATGCAGACCTTGGATGCTAGGCAGGACATGTTGGTGGTCGA
AGTGCCAAAACTCGGCAAGGACGCGTGCGCTAAGGCAATCAAGGAGTGGGGTCAAC
CGAAGTCTAAAATCACGCATCTAATATTTACATCTGCACTGACAACCGACATGCCGG
GTGCCGATTATCACTGCGCCAAGCTACTTGGATTGAGTCCACTGGTTAAGAGAGTTA
TGATGTATCAATTGGGGTGTTACGGAGGGGGCACAGTCCTCAGAATTGCTAAGGAT
ATTGCGGAAAATAACAAGGGCGCGAGGGTCCTTGCTGTATGTTGTGATATTATGGCC
TGTTTGTTTCGCGGGCCCTCGGATTCAGATTTGGAATTGCTTGTCGGACAGGCAATTT
TTGGTGACGGGGCCGCAGCAGTCATAGTGGGAGCCGAACCAGACGAAAGCGTGGGT
GAAAGACCAATCTTTGAGTTGGTTCTGACCGGACAAACGATCTTACCTAACTCGGAA
GGTACGATTGGAGGACATATTAGAGAAGCCGGCCTAATTTTCGATCTTCACAAAGAC
GTTCCAATGTTAATCTCCAATAACATAGAAAAGTGCTTGATAGAAGCATTTACTCCC
ATTGGTATTAGTGACTGGAACAGCATTTTCTGGATCACCCACCCTGGAGGAAAAGCT
ATACTCGATAAGGTTGAAGAGAAACTCGACTTGAAAAAGGAGAAATTCGTTGACTC
ACGACATGTGTTATCAGAGCACGGGAATATGAGTTCATCCACAGTCTTGTTCGTAAT
GGATGAATTGCGAAAACGCTCTCTTGAGGAGGGAAAGAGCACAACCGGTGACGGGT
TTGAGTGGGGCGTGCTATTCGGTTTTGGCCCAGGTTTGACTGTCGAGCGGGTTGTTG
TTCGTAGTGTACCAATTAAGTACGGAAGAAGGGCAAAGTTGTGA
CvOAC1 Gene
(SEQ ID NO: 97)
ATGGCAGTCAAACACCTAATAGTTCTCAAATTTAAAGACGAGATTACTGAAGCTCAG
AAGGAAGAGTTCTTTAAGACATATGTTAACTTAGTCAACATCATCCCCGCGATGAAG
GACGTCTACTGGGGCAAGGATGTGACGCAAAAAAATAAGGAAGAAGGATACACAC
ATATCGTTGAGGTGACCTTTGAGAGTGTGGAAACTATTCAAGATTATATTATTCACC
CAGCCCATGTAGGGTTCGGTGACGTTTATCGATCATTCTGGGAAAAGTTGCTTATAT
TTGATTACACCCCAAGAAAATTGAAGCCTAAGTGA
CvOACP1 Gene
(SEQ ID NO: 98)
ATGGCAGTCAAACACCTAATAGTTCTCAAATTTAAAGACGAGATTACTGAAGCTCAG
AAGGAAGAGTTCTTTAAGACATATGTTAACTTAGTCAACATCATCCCCGCGATGAAG
GACGTCTACTGGGGCAAGGATGTGACGCAAAAAAATAAGGAAGAAGGATACACAC
ATATCGTTGAGGTGACCTTTGAGAGTGTGGAAACTATTCAAGATTATATTATTCACC
CAGCCCATGTAGGGTTCGGTGACGTTTATCGATCATTCTGGGAAAAGTTGCTTATAT
TTGATTACACCCCAAGAAAATTGAAGCCTAAGGGAAGACGAGCTAAGTTGTGA
CvPTS1 Gene
(SEQ ID NO: 99)
ATGGGTTTATCGTCAGTGTGCACTTTTTCTTTTCAAACAAACTACCACACCCTCCTAA
ACCCTCACAATAATAACCCAAAAACCTCCTTGCTATGTTACAGACATCCAAAGACAC
CGATCAAGTATTCATACAACAATTTTCCCAGTAAACATTGCTCAACGAAGTCCTTCC
ACTTGCAAAACAAATGCAGCGAATCATTGTCGATAGCTAAAAACTCGATACGTGCG
GCAACCACTAACCAAACTGAGCCACCAGAGAGCGATAATCATTCAGTCGCCACCAA
GATTTTGAACTTTGGAAAAGCCTGTTGGAAACTTCAAAGGCCTTACACCATTATCGC
ATTTACCAGTTGCGCATGTGGTTTGTTCGGGAAGGAATTATTACACAACACAAATTT
GATCAGCTGGAGCCTAATGTTTAAGGCATTTTTCTTCTTAGTTGCAATTTTGTGTATA
GCTTCGTTTACAACGACCATTAATCAGATTTACGACCTTCACATCGATCGGATCAAT
AAACCAGACTTGCCCCTTGCCTCTGGGGAAATCTCTGTAAATACTGCATGGATCATG
CTGATAATCGTGGCTTTGTTTGGATTGATTATTACAATTAAGATGAAGGGGGGTCCA
TTATATATATTCGGGTACTGCTTCGGCATTTTCGGTGGTATCGTTTACTCCGTCCCAC
CCTTTAGATGGAAACAGAACCCCAGTACGGCCTTTCTACTCAATTTCTTGGCTCATAT
CATCACAAACTTCACATTCTATTATGCAAGCCGAGCGGCGCTTGGTTTGCCGTTCGA
ACTCAGACCGAGTTTTACATTTCTCCTTGCCTTCATGAAACTGATGGGACTGGCCCTT
GCATTGATCAAGGATGCGTCAGATGTCGAAGGCGACACTAAGTTCGGCATTCTGAC
GCTTGCTTCCAAGTATGGAAGTAGAAATCTAACGCTTTTTTGTTCAGGAATAGTGCT
ACTTAGTTATGTTGCTGCTATACTCGCTGGCATTATTTGGCCTCAGGCCTTCAACTCT
AACGTAATGTTGTTATCCCATGCTATTTTGGCGTTCTGGTTGATCTTGCAAACGCGAG
ATTTTGCACTCACTAACTACGACCCAGAGGCAGGAAGGCGCTTTTACGAGTTTATGT
GGAAGTTGTATTATGCCGAATACTTGGTTTATGTTTTCATTTGA
CVPTS1dN Gene
(SEQ ID NO: 100)
ATGGCGGCAACCACTAACCAAACTGAGCCACCAGAGAGCGATAATCATTCAGTCGC
CACCAAGATTTTGAACTTTGGAAAAGCCTGTTGGAAACTTCAAAGGCCTTACACCAT
TATCGCATTTACCAGTTGCGCATGTGGTTTGTTCGGGAAGGAATTATTACACAACAC
AAATTTGATCAGCTGGAGCCTAATGTTTAAGGCATTTTTCTTCTTAGTTGCAATTTTG
TGTATAGCTTCGTTTACAACGACCATTAATCAGATTTACGACCTTCACATCGATCGG
ATCAATAAACCAGACTTGCCCCTTGCCTCTGGGGAAATCTCTGTAAATACTGCATGG
ATCATGCTGATAATCGTGGCTTTGTTTGGATTGATTATTACAATTAAGATGAAGGGG
GGTCCATTATATATATTCGGGTACTGCTTCGGCATTTTCGGTGGTATCGTTTACTCCG
TCCCACCCTTTAGATGGAAACAGAACCCCAGTACGGCCTTTCTACTCAATTTCTTGG
CTCATATCATCACAAACTTCACATTCTATTATGCAAGCCGAGCGGCGCTTGGTTTGC
CGTTCGAACTCAGACCGAGTTTTACATTTCTCCTTGCCTTCATGAAACTGATGGGACT
GGCCCTTGCATTGATCAAGGATGCGTCAGATGTCGAAGGCGACACTAAGTTCGGCAT
TCTGACGCTTGCTTCCAAGTATGGAAGTAGAAATCTAACGCTTTTTTGTTCAGGAAT
AGTGCTACTTAGTTATGTTGCTGCTATACTCGCTGGCATTATTTGGCCTCAGGCCTTC
AACTCTAACGTAATGTTGTTATCCCATGCTATTTTGGCGTTCTGGTTGATCTTGCAAA
CGCGAGATTTTGCACTCACTAACTACGACCCAGAGGCAGGAAGGCGCTTTTACGAGT
TTATGTGGAAGTTGTATTATGCCGAATACTTGGTTTATGTTTTCATTTGA
CvPTS2 Gene
(SEQ ID NO: 101)
ATGGGGTTGTCCTTAGTTTGTACGTTCAGTTTCCAAACTAACTACCACACACTACTAA
ATCCGCACAACAAAAACCCGAAAAATTCATTGCTCTCCTATCAGCACCCAAAAACA
CCCATTATCAAGTCTAGTTACGACAACTTTCCATCAAAATACTGTCTAACGAAAAAC
TTTCATTTGTTGGGCTTAAATTCTCATAATCGTATTTCCAGTCAGTCCCGATCGATCA
GGGCCGGGAGTGACCAAATTGAAGGTTCTCCACATCATGAAAGTGACAATTCAATT
GCTACGAAGATTTTAAACTTTGGGCATACATGCTGGAAGCTACAGCGACCGTATGTA
GTTAAGGGGATGATCAGCATTGCCTGCGGCCTATTCGGAAGGGAACTCTTCAATAAT
AGACATCTTTTTTCTTGGGGTTTAATGTGGAAAGCTTTTTTCGCTTTGGTTCCTATCCT
TAGTTTTAACTTCTTCGCCGCTATTATGAATCAAATTTACGATGTTGACATCGACCGT
ATTAACAAACCCGATCTCCCCCTTGTTTCAGGCGAGATGTCCATTGAAACGGCATGG
ATTTTGTCCATCATTGTTGCGCTTACTGGCTTGATTGTTACCATTAAGCTTAAAAGCG
CTCCCTTGTTCGTTTTTATATACATTTTCGGCATTTTTGCCGGATTCGCATACAGTGTC
CCGCCTATACGTTGGAAACAATATCCATTCACGAACTTCTTGATCACGATCTCATCA
CATGTTGGATTGGCCTTTACGTCCTACAGTGCTACCACATCTGCCCTTGGATTGCCTT
TCGTTTGGAGGCCTGCCTTCTCGTTTATCATTGCATTTATGACAGTGATGGGAATGAC
TATCGCATTTGCTAAAGATATCAGCGACATAGAGGGCGATGCAAAATATGGGGTGA
GTACTGTTGCGACGAAGTTGGGCGCCCGAAATATGACCTTCGTTGTTTCCGGCGTTC
TTTTACTTAACTATTTAGTATCGATTAGCATCGGGATCATCTGGCCACAGGTGTTTAA
ATCAAATATTATGATCTTGTCGCATGCCATCCTAGCTTTCTGTCTTATATTTCAAACA
AGAGAATTAGCCCTAGCGAACTACGCCTCAGCACCAAGTCGTCAGTTCTTCGAATTT
ATATGGCTACTCTACTACGCCGAATACTTCGTCTATGTCTTCATTTAG
CvCBD1 Gene
(SEQ ID NO: 102)
ATGAAGTGTTCTACGTTTAGTTTTTGGTTTGTTTGTAAAATTATATTCTTCTTTTTTTC
CTTCAACATTCAGACATCAATCGCCAACCCAAGGGAAAACTTCCTTAAGTGTTTTCT
GCAGTACATCCCTAACAATGCAACAAACCTCAAGTTGGTGTACACTCAAAACAATC
CACTCTATATGAGCGTGCTTAATAGCACAATCCACAACTTGCGCTTCACGTCAGATA
CTACGCCTAAGCCACTAGTGATCGTTACACCATCACACGTCAGCCATATTCAAGGAA
CGATCCTATGTCTGAAAAAGGTCGGGTTGCAAATCAGGACTCGATCAGGAGGGCAC
GATAGTGAGGGAATGAGTTACATCTCGCAAGTACCCTTCGTGATAGTTGACTTGCGA
AATATGCGGTCTATTAAAATTGACGTACATAGCCAGACCGCCTGGGTTGAAGCAGG
GGCAACCTTGGGTGAAGTTTATTACTGGGTCAATGAAAAAAACGAAAACCTAAGTC
TTGCTGCTGGATATTGCCCCACCGTTTGCGCGGGTGGTCATTTTGGAGGCGGCGGAT
ATGGTCCGTTGATGAGAAATTATGGACTTGCAGCAGACAATATTATAGATGCCCACT
TGGTGAACGTTCATGGAAAGGTCTTGGACCGTAAGTCCATGGGTGAAGATCTTTTCT
GGGCCTTGAGAGGTGGTGGAGCGGAATCGTTTGGCATCATCGTTGCCTGGAAAATTA
GGTTGGTTGCGGTCCCGAAGAGTACAATGTTCTCCGTGAAGAAGATTATGGAAATAC
ATGAGCTTGTCAAGTTAGTTAACAAGTGGCAAAATATCGCTTATAAGTATGATAAAG
ACTTGCTTTTGATGACTCATTTTATTACGCGAAACATAACCGATAACCAGGGCAAGA
ACAAGACTGCTATTCACACGTACTTCTCCTCTGTATTTCTTGGAGGAGTAGACTCCTT
AGTTGACTTGATGAACAAGAGTTTCCCAGAATTGGGGATTAAGAAGACAGATTGCA
GACAATTATCGTGGATAGATACAATCATATTCTATAGCGGTGTCGTCAATTACGATA
CTGATAATTTTAATAAAGAAATCCTCCTAGATCGTTCAGCTGGGCAAAACGGGGCAT
TCAAAATTAAATTGGATTATGTGAAGAAACCAATTCCAGAGCTGGTGTTTGTTCAGA
TATTGGAAAAACTTTACGAAGAAGACATTGGCGCAGGTATGTACGCTTTGTATCCAT
ATGGAGGCATTATGGACGAGATCTCAGAGCTGGCGATCCCCTTCCCGCACAGAGCT
GGGATACTCTACGAGCTATGGTACATCTGCTCTTGGGAGAAACAAGAAGACAACGA
GAAACATCTCAATTGGATTCGGAACATATACAACTTTATGACCCCATACGTATCAAA
AAACCCGCGCTTAGCATACTTGAATTACAGAGACTTAGATATCGGTATCAATGATCC
TAAGAATCCTAACAATTACACCCAAGCCCGTATTTGGGGTGAGAAATATTTCGGCAA
GAATTTTGACAGATTAGTTAAGGTCAAAACACTCGTGGACCCCAACAACTTTTTCCG
AAACGAGCAGTCGATTCCACCACTACCCAGGCATAGACACTGA
CBD1dNS1
(SEQ ID NO: 103)
ATGTTCTTGAAACACATTTTTGTTGCTCTCGCTTTTGCCTTGTTAGCTGACGCTACCC
CAGCCCAGAAGAGATCTCCCGGCTTCGTTGCTTTAGACTTTGACATCGTCAAGGTTC
AAAAGAACGTGACTGCCAACGACGACGCCGCTGCCATTGTTGCCAAGAGACAGACC
AACCCAAGGGAAAACTTCCTTAAGTGTTTTCTGCAGTACATCCCTAACAATGCAACA
AACCTCAAGTTGGTGTACACTCAAAACAATCCACTCTATATGAGCGTGCTTAATAGC
ACAATCCACAACTTGCGCTTCACGTCAGATACTACGCCTAAGCCACTAGTGATCGTT
ACACCATCACACGTCAGCCATATTCAAGGAACGATCCTATGTCTGAAAAAGGTCGG
GTTGCAAATCAGGACTCGATCAGGAGGGCACGATAGTGAGGGAATGAGTTACATCT
CGCAAGTACCCTTCGTGATAGTTGACTTGCGAAATATGCGGTCTATTAAAATTGACG
TACATAGCCAGACCGCCTGGGTTGAAGCAGGGGCAACCTTGGGTGAAGTTTATTACT
GGGTCAATGAAAAAAACGAAAACCTAAGTCTTGCTGCTGGATATTGCCCCACCGTTT
GCGCGGGTGGTCATTTTGGAGGCGGCGGATATGGTCCGTTGATGAGAAATTATGGA
CTTGCAGCAGACAATATTATAGATGCCCACTTGGTGAACGTTCATGGAAAGGTCTTG
GACCGTAAGTCCATGGGTGAAGATCTTTTCTGGGCCTTGAGAGGTGGTGGAGCGGA
ATCGTTTGGCATCATCGTTGCCTGGAAAATTAGGTTGGTTGCGGTCCCGAAGAGTAC
AATGTTCTCCGTGAAGAAGATTATGGAAATACATGAGCTTGTCAAGTTAGTTAACAA
GTGGCAAAATATCGCTTATAAGTATGATAAAGACTTGCTTTTGATGACTCATTTTATT
ACGCGAAACATAACCGATAACCAGGGCAAGAACAAGACTGCTATTCACACGTACTT
CTCCTCTGTATTTCTTGGAGGAGTAGACTCCTTAGTTGACTTGATGAACAAGAGTTTC
CCAGAATTGGGGATTAAGAAGACAGATTGCAGACAATTATCGTGGATAGATACAAT
CATATTCTATAGCGGTGTCGTCAATTACGATACTGATAATTTTAATAAAGAAATCCT
CCTAGATCGTTCAGCTGGGCAAAACGGGGCATTCAAAATTAAATTGGATTATGTGAA
GAAACCAATTCCAGAGCTGGTGTTTGTTCAGATATTGGAAAAACTTTACGAAGAAG
ACATTGGCGCAGGTATGTACGCTTTGTATCCATATGGAGGCATTATGGACGAGATCT
CAGAGCTGGCGATCCCCTTCCCGCACAGAGCTGGGATACTCTACGAGCTATGGTACA
TCTGCTCTTGGGAGAAACAAGAAGACAACGAGAAACATCTCAATTGGATTCGGAAC
ATATACAACTTTATGACCCCATACGTATCAAAAAACCCGCGCTTAGCATACTTGAAT
TACAGAGACTTAGATATCGGTATCAATGATCCTAAGAATCCTAACAATTACACCCAA
GCCCGTATTTGGGGTGAGAAATATTTCGGCAAGAATTTTGACAGATTAGTTAAGGTC
AAAACACTCGTGGACCCCAACAACTTTTTCCGAAACGAGCAGTCGATTCCACCACTA
CCCAGGCATAGACACTGA
CBD1dNS2
(SEQ ID NO: 104)
ATGCAATTGTCATTGTCAGTTTTGTCAACAGTTGCAACAGCATTGTTGTCATTGACAA
CAGCAGTTGATGCAAAGTCACATAACCCAAGGGAAAACTTCCTTAAGTGTTTTCTGC
AGTACATCCCTAACAATGCAACAAACCTCAAGTTGGTGTACACTCAAAACAATCCA
CTCTATATGAGCGTGCTTAATAGCACAATCCACAACTTGCGCTTCACGTCAGATACT
ACGCCTAAGCCACTAGTGATCGTTACACCATCACACGTCAGCCATATTCAAGGAACG
ATCCTATGTCTGAAAAAGGTCGGGTTGCAAATCAGGACTCGATCAGGAGGGCACGA
TAGTGAGGGAATGAGTTACATCTCGCAAGTACCCTTCGTGATAGTTGACTTGCGAAA
TATGCGGTCTATTAAAATTGACGTACATAGCCAGACCGCCTGGGTTGAAGCAGGGG
CAACCTTGGGTGAAGTTTATTACTGGGTCAATGAAAAAAACGAAAACCTAAGTCTTG
CTGCTGGATATTGCCCCACCGTTTGCGCGGGTGGTCATTTTGGAGGCGGCGGATATG
GTCCGTTGATGAGAAATTATGGACTTGCAGCAGACAATATTATAGATGCCCACTTGG
TGAACGTTCATGGAAAGGTCTTGGACCGTAAGTCCATGGGTGAAGATCTTTTCTGGG
CCTTGAGAGGTGGTGGAGCGGAATCGTTTGGCATCATCGTTGCCTGGAAAATTAGGT
TGGTTGCGGTCCCGAAGAGTACAATGTTCTCCGTGAAGAAGATTATGGAAATACATG
AGCTTGTCAAGTTAGTTAACAAGTGGCAAAATATCGCTTATAAGTATGATAAAGACT
TGCTTTTGATGACTCATTTTATTACGCGAAACATAACCGATAACCAGGGCAAGAACA
AGACTGCTATTCACACGTACTTCTCCTCTGTATTTCTTGGAGGAGTAGACTCCTTAGT
TGACTTGATGAACAAGAGTTTCCCAGAATTGGGGATTAAGAAGACAGATTGCAGAC
AATTATCGTGGATAGATACAATCATATTCTATAGCGGTGTCGTCAATTACGATACTG
ATAATTTTAATAAAGAAATCCTCCTAGATCGTTCAGCTGGGCAAAACGGGGCATTCA
AAATTAAATTGGATTATGTGAAGAAACCAATTCCAGAGCTGGTGTTTGTTCAGATAT
TGGAAAAACTTTACGAAGAAGACATTGGCGCAGGTATGTACGCTTTGTATCCATATG
GAGGCATTATGGACGAGATCTCAGAGCTGGCGATCCCCTTCCCGCACAGAGCTGGG
ATACTCTACGAGCTATGGTACATCTGCTCTTGGGAGAAACAAGAAGACAACGAGAA
ACATCTCAATTGGATTCGGAACATATACAACTTTATGACCCCATACGTATCAAAAAA
CCCGCGCTTAGCATACTTGAATTACAGAGACTTAGATATCGGTATCAATGATCCTAA
GAATCCTAACAATTACACCCAAGCCCGTATTTGGGGTGAGAAATATTTCGGCAAGA
ATTTTGACAGATTAGTTAAGGTCAAAACACTCGTGGACCCCAACAACTTTTTCCGAA
ACGAGCAGTCGATTCCACCACTACCCAGGCATAGACACTGA
CBD1dNV1
(SEQ ID NO: 105)
ATGCAATTGTCCTTGTCGGTTTTATCAACCGTTGCCACGGCCTTGTTGTCCCTAACCA
CCGCCGTCGATGCTAAGTCCCACAACATCAAGTTGTCCAAGTTGTCCAACGAAGAA
ACATTGGACGCCTCCACATTCCAAGAATACACGAGCTCCTTGGCCAACAAGTACATG
AACTTGTTCAACGCCGCTCACGGTAACCCAACCAGCTTTGGCTTGCAACACGTCTTG
TCCAACCAAGAAGCTGAAGTCCCATTCGTTACCCCACAAAAGGGTGGCAACCCAAG
GGAAAACTTCCTTAAGTGTTTTCTGCAGTACATCCCTAACAATGCAACAAACCTCAA
GTTGGTGTACACTCAAAACAATCCACTCTATATGAGCGTGCTTAATAGCACAATCCA
CAACTTGCGCTTCACGTCAGATACTACGCCTAAGCCACTAGTGATCGTTACACCATC
ACACGTCAGCCATATTCAAGGAACGATCCTATGTCTGAAAAAGGTCGGGTTGCAAA
TCAGGACTCGATCAGGAGGGCACGATAGTGAGGGAATGAGTTACATCTCGCAAGTA
CCCTTCGTGATAGTTGACTTGCGAAATATGCGGTCTATTAAAATTGACGTACATAGC
CAGACCGCCTGGGTTGAAGCAGGGGCAACCTTGGGTGAAGTTTATTACTGGGTCAAT
GAAAAAAACGAAAACCTAAGTCTTGCTGCTGGATATTGCCCCACCGTTTGCGCGGGT
GGTCATTTTGGAGGCGGCGGATATGGTCCGTTGATGAGAAATTATGGACTTGCAGCA
GACAATATTATAGATGCCCACTTGGTGAACGTTCATGGAAAGGTCTTGGACCGTAAG
TCCATGGGTGAAGATCTTTTCTGGGCCTTGAGAGGTGGTGGAGCGGAATCGTTTGGC
ATCATCGTTGCCTGGAAAATTAGGTTGGTTGCGGTCCCGAAGAGTACAATGTTCTCC
GTGAAGAAGATTATGGAAATACATGAGCTTGTCAAGTTAGTTAACAAGTGGCAAAA
TATCGCTTATAAGTATGATAAAGACTTGCTTTTGATGACTCATTTTATTACGCGAAAC
ATAACCGATAACCAGGGCAAGAACAAGACTGCTATTCACACGTACTTCTCCTCTGTA
TTTCTTGGAGGAGTAGACTCCTTAGTTGACTTGATGAACAAGAGTTTCCCAGAATTG
GGGATTAAGAAGACAGATTGCAGACAATTATCGTGGATAGATACAATCATATTCTAT
AGCGGTGTCGTCAATTACGATACTGATAATTTTAATAAAGAAATCCTCCTAGATCGT
TCAGCTGGGCAAAACGGGGCATTCAAAATTAAATTGGATTATGTGAAGAAACCAAT
TCCAGAGCTGGTGTTTGTTCAGATATTGGAAAAACTTTACGAAGAAGACATTGGCGC
AGGTATGTACGCTTTGTATCCATATGGAGGCATTATGGACGAGATCTCAGAGCTGGC
GATCCCCTTCCCGCACAGAGCTGGGATACTCTACGAGCTATGGTACATCTGCTCTTG
GGAGAAACAAGAAGACAACGAGAAACATCTCAATTGGATTCGGAACATATACAACT
TTATGACCCCATACGTATCAAAAAACCCGCGCTTAGCATACTTGAATTACAGAGACT
TAGATATCGGTATCAATGATCCTAAGAATCCTAACAATTACACCCAAGCCCGTATTT
GGGGTGAGAAATATTTCGGCAAGAATTTTGACAGATTAGTTAAGGTCAAAACACTC
GTGGACCCCAACAACTTTTTCCGAAACGAGCAGTCGATTCCACCACTACCCAGGCAT
AGACACTGA
CvCBD1dNP1
(SEQ ID NO: 106)
ATGAACCCAAGGGAAAACTTCCTTAAGTGTTTTCTGCAGTACATCCCTAACAATGCA
ACAAACCTCAAGTTGGTGTACACTCAAAACAATCCACTCTATATGAGCGTGCTTAAT
AGCACAATCCACAACTTGCGCTTCACGTCAGATACTACGCCTAAGCCACTAGTGATC
GTTACACCATCACACGTCAGCCATATTCAAGGAACGATCCTATGTCTGAAAAAGGTC
GGGTTGCAAATCAGGACTCGATCAGGAGGGCACGATAGTGAGGGAATGAGTTACAT
CTCGCAAGTACCCTTCGTGATAGTTGACTTGCGAAATATGCGGTCTATTAAAATTGA
CGTACATAGCCAGACCGCCTGGGTTGAAGCAGGGGCAACCTTGGGTGAAGTTTATT
ACTGGGTCAATGAAAAAAACGAAAACCTAAGTCTTGCTGCTGGATATTGCCCCACC
GTTTGCGCGGGTGGTCATTTTGGAGGCGGCGGATATGGTCCGTTGATGAGAAATTAT
GGACTTGCAGCAGACAATATTATAGATGCCCACTTGGTGAACGTTCATGGAAAGGTC
TTGGACCGTAAGTCCATGGGTGAAGATCTTTTCTGGGCCTTGAGAGGTGGTGGAGCG
GAATCGTTTGGCATCATCGTTGCCTGGAAAATTAGGTTGGTTGCGGTCCCGAAGAGT
ACAATGTTCTCCGTGAAGAAGATTATGGAAATACATGAGCTTGTCAAGTTAGTTAAC
AAGTGGCAAAATATCGCTTATAAGTATGATAAAGACTTGCTTTTGATGACTCATTTT
ATTACGCGAAACATAACCGATAACCAGGGCAAGAACAAGACTGCTATTCACACGTA
CTTCTCCTCTGTATTTCTTGGAGGAGTAGACTCCTTAGTTGACTTGATGAACAAGAGT
TTCCCAGAATTGGGGATTAAGAAGACAGATTGCAGACAATTATCGTGGATAGATAC
AATCATATTCTATAGCGGTGTCGTCAATTACGATACTGATAATTTTAATAAAGAAAT
CCTCCTAGATCGTTCAGCTGGGCAAAACGGGGCATTCAAAATTAAATTGGATTATGT
GAAGAAACCAATTCCAGAGCTGGTGTTTGTTCAGATATTGGAAAAACTTTACGAAG
AAGACATTGGCGCAGGTATGTACGCTTTGTATCCATATGGAGGCATTATGGACGAGA
TCTCAGAGCTGGCGATCCCCTTCCCGCACAGAGCTGGGATACTCTACGAGCTATGGT
ACATCTGCTCTTGGGAGAAACAAGAAGACAACGAGAAACATCTCAATTGGATTCGG
AACATATACAACTTTATGACCCCATACGTATCAAAAAACCCGCGCTTAGCATACTTG
AATTACAGAGACTTAGATATCGGTATCAATGATCCTAAGAATCCTAACAATTACACC
CAAGCCCGTATTTGGGGTGAGAAATATTTCGGCAAGAATTTTGACAGATTAGTTAAG
GTCAAAACACTCGTGGACCCCAACAACTTTTTCCGAAACGAGCAGTCGATTCCACCA
CTACCCAGGCATAGACACGGAAGAAGGGCAAAGTTGTAA
CvTHC1 Gene
(SEQ ID NO: 107)
ATGAATTGTTCAGCATTTAGTTTTTGGTTTGTTTGTAAGATTATTTTCTTCTTTTTGTC
ATTTAACATTCAAATTTCAATTGCAAACCCACAAGAAAACTTTTTGAAGTGTTTTTCA
GAATACATTCCAAACAATCCAGCTAACCCAAAGTTTATTTACACACAACATGATCAA
TTGTACATGTCAGTTTTGAACTCAACAATTCAAAACTTGAGATTTACATCAGATACC
ACACCAAAGCCATTGGTTATTGTTACACCATCAAACGTTTCCCATATTCAAGCATCA
ATCTTGTGTTCAAAGAAGGTTGGATTGCAAATTAGAACCAGATCAGGAGGACACGA
TGCAGAAGGAATGTCATACATTTCACAAGTTCCATTCGTTGTTGTTGATTTGAGAAA
CATGCACTCAATTAAGATTGATGTTCATTCACAAACAGCATGGGTTGAAGCAGGAGC
AACATTGGGTGAAGTTTACTACTGGATTAACGAAAAGAACGAAAACTTCAGTTTTCC
AGGAGGTTACTGTCCAACAGTTGGAGTTGGAGGACATTTTTCAGGTGGAGGATACG
GAGCATTGATGAGAAACTACGGATTGGCAGCAGATAACATTATTGATGCACACTTG
GTTAACGTTGATGGAAAGGTTTTGGATAGAAAGTCAATGGGAGAAGATTTGTTTTGG
GCAATTAGAGGAGGTGGTGGAGAGAACTTTGGAATTATTGCAGCATGGAAGATCAA
GTTGGTTGCAGTTCCATCAAAGTCAACAATCTTTTCAGTTAAGAAGAACATGGAAAT
TCATGGTTTGGTTAAGTTGTTTAACAAGTGGCAAAACATTGCATACAAGTACGATAA
GGATTTGGTTTTGATGACACATTTTATTACAAAGAACATTACAGATAACCATGGAAA
GAACAAGACAACAGTTCACGGATACTTTTCATCAATTTTTCACGGAGGAGTTGATTC
ATTGGTTGACTTGATGAACAAGTCATTTCCAGAATTGGGAATCAAGAAGACAGATTG
TAAGGAATTTTCATGGATTGATACAACAATTTTCTACTCAGGAGTTGTTAACTTTAAC
ACAGCAAACTTTAAGAAGGAAATTTTGTTGGACAGATCAGCAGGAAAGAAGACCGC
ATTTTCCATTAAGTTGGATTACGTTAAGAAACCAATTCCAGAAACAGCAATGGTTAA
GATTTTGGAAAAGTTGTACGAAGAAGATGTTGGTGTTGGAATGTACGTTTTGTACCC
ATACGGAGGAATTATGGAAGAAATCTCAGAATCAGCAATTCCATTTCCACATAGAG
CAGGTATTATGTACGAATTGTGGTACACAGCATCATGGGAAAAGCAAGAAGATAAT
GAAAAGCATATTAACTGGGTTAGATCAGTTTACAACTTTACAACACCATACGTTTCA
CAAAACCCAAGATTGGCATACTTGAACTACAGAGATTTGGATTTGGGAAAGACAAA
CCCAGAATCACCAAACAACTATACACAAGCTAGAATTTGGGGAGAAAAGTACTTTG
GTAAGAACTTCAACAGATTGGTTAAAGTTAAGACAAAGGCAGATCCAAATAACTTC
TTTAGAAACGAACAATCAATTCCACCATTGCCACCACATCATCATTAA
THC1dNS1 Gene
(SEQ ID NO: 108)
ATGTTCTTGAAACACATTTTTGTTGCTCTCGCTTTTGCCTTGTTAGCTGACGCTACCC
CAGCCCAGAAGAGATCTCCCGGCTTCGTTGCTTTAGACTTTGACATCGTCAAGGTTC
AAAAGAACGTGACTGCCAACGACGACGCCGCTGCCATTGTTGCCAAGAGACAGACC
AACCCACAAGAAAACTTTTTGAAGTGTTTTTCAGAATACATTCCAAACAATCCAGCT
AACCCAAAGTTTATTTACACACAACATGATCAATTGTACATGTCAGTTTTGAACTCA
ACAATTCAAAACTTGAGATTTACATCAGATACCACACCAAAGCCATTGGTTATTGTT
ACACCATCAAACGTTTCCCATATTCAAGCATCAATCTTGTGTTCAAAGAAGGTTGGA
TTGCAAATTAGAACCAGATCAGGAGGACACGATGCAGAAGGAATGTCATACATTTC
ACAAGTTCCATTCGTTGTTGTTGATTTGAGAAACATGCACTCAATTAAGATTGATGTT
CATTCACAAACAGCATGGGTTGAAGCAGGAGCAACATTGGGTGAAGTTTACTACTG
GATTAACGAAAAGAACGAAAACTTCAGTTTTCCAGGAGGTTACTGTCCAACAGTTG
GAGTTGGAGGACATTTTTCAGGTGGAGGATACGGAGCATTGATGAGAAACTACGGA
TTGGCAGCAGATAACATTATTGATGCACACTTGGTTAACGTTGATGGAAAGGTTTTG
GATAGAAAGTCAATGGGAGAAGATTTGTTTTGGGCAATTAGAGGAGGTGGTGGAGA
GAACTTTGGAATTATTGCAGCATGGAAGATCAAGTTGGTTGCAGTTCCATCAAAGTC
AACAATCTTTTCAGTTAAGAAGAACATGGAAATTCATGGTTTGGTTAAGTTGTTTAA
CAAGTGGCAAAACATTGCATACAAGTACGATAAGGATTTGGTTTTGATGACACATTT
TATTACAAAGAACATTACAGATAACCATGGAAAGAACAAGACAACAGTTCACGGAT
ACTTTTCATCAATTTTTCACGGAGGAGTTGATTCATTGGTTGACTTGATGAACAAGTC
ATTTCCAGAATTGGGAATCAAGAAGACAGATTGTAAGGAATTTTCATGGATTGATAC
AACAATTTTCTACTCAGGAGTTGTTAACTTTAACACAGCAAACTTTAAGAAGGAAAT
TTTGTTGGACAGATCAGCAGGAAAGAAGACCGCATTTTCCATTAAGTTGGATTACGT
TAAGAAACCAATTCCAGAAACAGCAATGGTTAAGATTTTGGAAAAGTTGTACGAAG
AAGATGTTGGTGTTGGAATGTACGTTTTGTACCCATACGGAGGAATTATGGAAGAAA
TCTCAGAATCAGCAATTCCATTTCCACATAGAGCAGGTATTATGTACGAATTGTGGT
ACACAGCATCATGGGAAAAGCAAGAAGATAATGAAAAGCATATTAACTGGGTTAGA
TCAGTTTACAACTTTACAACACCATACGTTTCACAAAACCCAAGATTGGCATACTTG
AACTACAGAGATTTGGATTTGGGAAAGACAAACCCAGAATCACCAAACAACTATAC
ACAAGCTAGAATTTGGGGAGAAAAGTACTTTGGTAAGAACTTCAACAGATTGGTTA
AAGTTAAGACAAAGGCAGATCCAAATAACTTCTTTAGAAACGAACAATCAATTCCA
CCATTGCCACCACATCATCATTAATAA
THC1dNS2
(SEQ ID NO: 109)
ATGCAATTGTCATTGTCAGTTTTGTCAACAGTTGCAACAGCATTGTTGTCATTGACAA
CAGCAGTTGATGCAAAGTCACATAACCCACAAGAAAACTTTTTGAAGTGTTTTTCAG
AATACATTCCAAACAATCCAGCTAACCCAAAGTTTATTTACACACAACATGATCAAT
TGTACATGTCAGTTTTGAACTCAACAATTCAAAACTTGAGATTTACATCAGATACCA
CACCAAAGCCATTGGTTATTGTTACACCATCAAACGTTTCCCATATTCAAGCATCAA
TCTTGTGTTCAAAGAAGGTTGGATTGCAAATTAGAACCAGATCAGGAGGACACGAT
GCAGAAGGAATGTCATACATTTCACAAGTTCCATTCGTTGTTGTTGATTTGAGAAAC
ATGCACTCAATTAAGATTGATGTTCATTCACAAACAGCATGGGTTGAAGCAGGAGC
AACATTGGGTGAAGTTTACTACTGGATTAACGAAAAGAACGAAAACTTCAGTTTTCC
AGGAGGTTACTGTCCAACAGTTGGAGTTGGAGGACATTTTTCAGGTGGAGGATACG
GAGCATTGATGAGAAACTACGGATTGGCAGCAGATAACATTATTGATGCACACTTG
GTTAACGTTGATGGAAAGGTTTTGGATAGAAAGTCAATGGGAGAAGATTTGTTTTGG
GCAATTAGAGGAGGTGGTGGAGAGAACTTTGGAATTATTGCAGCATGGAAGATCAA
GTTGGTTGCAGTTCCATCAAAGTCAACAATCTTTTCAGTTAAGAAGAACATGGAAAT
TCATGGTTTGGTTAAGTTGTTTAACAAGTGGCAAAACATTGCATACAAGTACGATAA
GGATTTGGTTTTGATGACACATTTTATTACAAAGAACATTACAGATAACCATGGAAA
GAACAAGACAACAGTTCACGGATACTTTTCATCAATTTTTCACGGAGGAGTTGATTC
ATTGGTTGACTTGATGAACAAGTCATTTCCAGAATTGGGAATCAAGAAGACAGATTG
TAAGGAATTTTCATGGATTGATACAACAATTTTCTACTCAGGAGTTGTTAACTTTAAC
ACAGCAAACTTTAAGAAGGAAATTTTGTTGGACAGATCAGCAGGAAAGAAGACCGC
ATTTTCCATTAAGTTGGATTACGTTAAGAAACCAATTCCAGAAACAGCAATGGTTAA
GATTTTGGAAAAGTTGTACGAAGAAGATGTTGGTGTTGGAATGTACGTTTTGTACCC
ATACGGAGGAATTATGGAAGAAATCTCAGAATCAGCAATTCCATTTCCACATAGAG
CAGGTATTATGTACGAATTGTGGTACACAGCATCATGGGAAAAGCAAGAAGATAAT
GAAAAGCATATTAACTGGGTTAGATCAGTTTACAACTTTACAACACCATACGTTTCA
CAAAACCCAAGATTGGCATACTTGAACTACAGAGATTTGGATTTGGGAAAGACAAA
CCCAGAATCACCAAACAACTATACACAAGCTAGAATTTGGGGAGAAAAGTACTTTG
GTAAGAACTTCAACAGATTGGTTAAAGTTAAGACAAAGGCAGATCCAAATAACTTC
TTTAGAAACGAACAATCAATTCCACCATTGCCACCACATCATCATTAATAA
THC1dNV1
(SEQ ID NO: 110)
ATGCAATTGTCCTTGTCGGTTTTATCAACCGTTGCCACGGCCTTGTTGTCCCTAACCA
CCGCCGTCGATGCTAAGTCCCACAACATCAAGTTGTCCAAGTTGTCCAACGAAGAA
ACATTGGACGCCTCCACATTCCAAGAATACACGAGCTCCTTGGCCAACAAGTACATG
AACTTGTTCAACGCCGCTCACGGTAACCCAACCAGCTTTGGCTTGCAACACGTCTTG
TCCAACCAAGAAGCTGAAGTCCCATTCGTTACCCCACAAAAGGGTGGCAACCCACA
AGAAAACTTTTTGAAGTGTTTTTCAGAATACATTCCAAACAATCCAGCTAACCCAAA
GTTTATTTACACACAACATGATCAATTGTACATGTCAGTTTTGAACTCAACAATTCA
AAACTTGAGATTTACATCAGATACCACACCAAAGCCATTGGTTATTGTTACACCATC
AAACGTTTCCCATATTCAAGCATCAATCTTGTGTTCAAAGAAGGTTGGATTGCAAAT
TAGAACCAGATCAGGAGGACACGATGCAGAAGGAATGTCATACATTTCACAAGTTC
CATTCGTTGTTGTTGATTTGAGAAACATGCACTCAATTAAGATTGATGTTCATTCACA
AACAGCATGGGTTGAAGCAGGAGCAACATTGGGTGAAGTTTACTACTGGATTAACG
AAAAGAACGAAAACTTCAGTTTTCCAGGAGGTTACTGTCCAACAGTTGGAGTTGGA
GGACATTTTTCAGGTGGAGGATACGGAGCATTGATGAGAAACTACGGATTGGCAGC
AGATAACATTATTGATGCACACTTGGTTAACGTTGATGGAAAGGTTTTGGATAGAAA
GTCAATGGGAGAAGATTTGTTTTGGGCAATTAGAGGAGGTGGTGGAGAGAACTTTG
GAATTATTGCAGCATGGAAGATCAAGTTGGTTGCAGTTCCATCAAAGTCAACAATCT
TTTCAGTTAAGAAGAACATGGAAATTCATGGTTTGGTTAAGTTGTTTAACAAGTGGC
AAAACATTGCATACAAGTACGATAAGGATTTGGTTTTGATGACACATTTTATTACAA
AGAACATTACAGATAACCATGGAAAGAACAAGACAACAGTTCACGGATACTTTTCA
TCAATTTTTCACGGAGGAGTTGATTCATTGGTTGACTTGATGAACAAGTCATTTCCA
GAATTGGGAATCAAGAAGACAGATTGTAAGGAATTTTCATGGATTGATACAACAAT
TTTCTACTCAGGAGTTGTTAACTTTAACACAGCAAACTTTAAGAAGGAAATTTTGTT
GGACAGATCAGCAGGAAAGAAGACCGCATTTTCCATTAAGTTGGATTACGTTAAGA
AACCAATTCCAGAAACAGCAATGGTTAAGATTTTGGAAAAGTTGTACGAAGAAGAT
GTTGGTGTTGGAATGTACGTTTTGTACCCATACGGAGGAATTATGGAAGAAATCTCA
GAATCAGCAATTCCATTTCCACATAGAGCAGGTATTATGTACGAATTGTGGTACACA
GCATCATGGGAAAAGCAAGAAGATAATGAAAAGCATATTAACTGGGTTAGATCAGT
TTACAACTTTACAACACCATACGTTTCACAAAACCCAAGATTGGCATACTTGAACTA
CAGAGATTTGGATTTGGGAAAGACAAACCCAGAATCACCAAACAACTATACACAAG
CTAGAATTTGGGGAGAAAAGTACTTTGGTAAGAACTTCAACAGATTGGTTAAAGTTA
AGACAAAGGCAGATCCAAATAACTTCTTTAGAAACGAACAATCAATTCCACCATTG
CCACCACATCATCATTAATAA
CvCBC1 Gene
(SEQ ID NO: 111)
ATGAATTGTAGCACTTTCTCATTCTGGTTTGTTTGTAAGATTATTTTCTTTTTCTTGTC
ATTTAACATTCAAATTTCAATTGCAAACCCACAAGAGAACTTTTTGAAGTGTTTCTC
AGAATACATTCCAAACAACCCAGCTAACCCAAAGTTTATTTACACCCAACACGATCA
ATTGTACATGTCAGTTTTGAACTCAACAATTCAAAACTTGAGATTTACATCAGATAC
AACACCAAAGCCATTGGTTATTGTTACACCATCAAACGTTAGTCATATTCAAGCATC
AATCTTGTGTTCAAAGAAGGTTGGATTGCAAATTAGAACTAGATCAGGAGGACATG
ATGCAGAAGGATTGTCATACATTTCACAAGTTCCATTTGCAATTGTTGATTTGAGAA
ACATGCACACAGTTAAGGTTGATATTCATTCACAAACAGCATGGGTTGAAGCAGGA
GCAACATTGGGTGAAGTTTACTACTGGATTAACGAAATGAACGAAAACTTCTCATTT
CCAGGAGGATACTGTCCAACAGTTGGTGTTGGAGGACACTTTTCAGGTGGTGGATAC
GGAGCATTGATGAGAAACTACGGATTGGCAGCAGATAACATTATTGATGCACATTT
GGTTAACGTTGATGGAAAGGTTTTGGATAGAAAGTCAATGGGAGAAGATTTGTTTTG
GGCAATTAGAGGAGGTGGAGGAGAAAACTTTGGAATCATTGCAGCATGTAAGATCA
AGTTGGTTGTTGTTCCATCAAAGGCAACAATCTTTTCAGTTAAGAAGAACATGGAAA
TCCATGGATTGGTTAAGTTGTTTAACAAGTGGCAAAACATTGCATACAAGTACGATA
AGGATTTGATGTTGACAACACATTTTAGAACAAGAAACATTACAGATAACCACGGA
AAGAATAAGACAACAGTTCATGGATACTTTTCATCAATTTTCTTGGGAGGAGTTGAT
TCATTGGTTGACTTGATGAACAAGAGTTTTCCAGAATTGGGAATCAAGAAGACAGAT
TGTAAGGAATTGTCATGGATCGATACAACCATTTTCTACTCAGGAGTTGTTAACTAC
AACACAGCTAACTTTAAGAAGGAAATTTTGTTGGACAGATCAGCAGGTAAAAAGAC
AGCATTTTCAATTAAGTTGGATTACGTTAAGAAATTGATTCCAGAAACAGCAATGGT
TAAGATTTTGGAAAAGTTGTACGAAGAAGAAGTTGGAGTTGGAATGTACGTTTTGTA
CCCATACGGAGGAATTATGGATGAAATTTCAGAATCAGCAATTCCATTTCCACATAG
AGCAGGTATTATGTACGAATTGTGGTACACAGCAACATGGGAAAAGCAAGAAGATA
ACGAAAAGCATATTAACTGGGTTAGATCAGTTTACAACTTTACAACCCCATACGTTT
CACAAAACCCAAGATTGGCATACTTGAACTACAGAGATTTGGATTTGGGAAAGACA
AACCCAGAATCACCAAATAACTACACACAAGCTAGAATTTGGGGAGAAAAGTACTT
TGGTAAGAACTTTAACAGATTGGTGAAGGTTAAGACAAAGGCAGACCCAAACAATT
TCTTTAGAAACGAACAATCAATTCCACCATTGCCACCAAGACATCATTAA

The following plasmids were constructed using modern molecular biology techniques described herein.

TABLE 1

| | | Important |
|---|---|---|
| Plasmid | Seq ID | Features |
| pLD1 | 157 | Empty URA3 multi integration |
| pLD10 | 158 | PPOX4-CvTKS1 URA3 multi |
| | | integration |
| pLD12 | 159 | PPOX4-CvOAC1 URA3 multi |
| | | integration |
| pLD14 | 160 | PPOX4-CvOAC1P URA3 multi |
| | | integration |
| pLD16 | 161 | PPOX4-CvHXS1 URA3 multi |
| | | integration |
| pLD19 | 180 | PPOX4-PTS1 URA3 multi integration |
| pLD20 | 162 | PPOX4-CvCBD1 URA3 multi integration |
| pLD22 | 163 | PPOX4-CvACO1P URA3 multi integration |
| pLD24 | 164 | PPOX4-CvTKS1P URA3 multi integration |
| pLD26 | 181 | PPOX4-PTS1dN URA3 multi integration |
| pLD56 | 165 | PPOX4-CvPTS2 URA3 multi integration |
| pLD111 | 169 | PPOX4-CvTHC1 URA3 multi integration |
| pLD112 | 170 | PPOX4-CvCBC1 URA3 multi integration |
| pLD125 | 172 | PPOX4-CvCBD1dNS1 URA3 multi |
| | | integration |
| pLD127 | 173 | PPOX4-CvCBD1dNV1 URA3 multi |
| | | integration |
| pLD139 | 179 | PPOX4-CvCBD1dNP1 URA3 multi |
| | | integration |

### Example 2—Construction of Plasmids for Yarrowia lipolytica

Protein sequence was reverse translated into DNA sequence to reflect the use of a universal genetic code and codon usage in Yarrowia lipolytica, reduction of secondary structure, and absence of certain restriction enzyme recognition sites. Other DNA sequences that encode the same protein may also be used. The following sequences were used as open reading frames for genes used to genetically modify Yarrowia lipolytica.

YlACO1P Gene
(SEQ ID NO: 112)
ATGACCGAAGTAGTTGACAGAGCCTCATCCCCCGC
ATCCCCTGGCTCAACTACGGCCGCCGCAGACGGTG
CTAAGGTGGCCGTCGAGCCCCGAGTAGATGTGGCT
GCGCTGGGAGAGCAGCTGCTGGGCCGATGGGCTGA
TATCCGTCTCCACGCCCGGGACCTTGCGGGACGAG
AGGTAGTTCAGAAGGTGGAGGGTCTGACTCATACA
GAGCACCGCTCTCGCGTCTTTGGCCAGCTCAAGTA
CTTGGTCGATAACAACGCAGTTCACCGAGCCTTTC
CTTCTCGACTGGGTGGTAGTGACGACCACGGCGGA
AACATCGCTGGTTTTGAGGAGCTTGTCACGGCGGA
CCCCTCCCTCCAGATCAAGGCCGGCGTCCAGTGGG
GACTGTTCGGCTCCGCTGTTATGCACTTGGGAACT
AGGGAGCACCACGACAAGTGGCTCCCAGGCATCAT
GTCTCTGGAAATCCCTGGTTGCTTTGCCATGACTG
AGACTGGCCATGGCTCCGATGTCGCTTCCATTGCT
ACAACGGCCACCTATGATGAGGAAACCCAGGAGTT
CGTTATTGACACCCCGTTCCGAGCCGCCTGGAAGG
ACTACATTGGAAACGCCGCTAACGACGGTTTGGCC
GCTGTCGTGTTTGCCCAACTGATTACTCGAAAGGT
TAACCATGGAGTGCACGCCTTCTACGTCGATCTGA
GAGATCCCGCCACCGGAGACTTTCTCCCTGGTATT
GGTGGAGAGGACGACGGTATTAAGGGGGGACTAAA
CGGAATTGATAACGGACGTCTCCATTTCACCAATG
TTCGCATTCCCCGAACCAACCTGCTTAACCGTTAC
GGCGATGTTGCCGTCGATGGCACCTACAGCTCAAC
CATCGAATCTCCGGGGCGAAGATTCTTTACAATGC
TAGGTACGCTGGTCCAGGGCCGAGTCAGCCTGGAC
GGTGCTGCAGTGGCTGCATCGAAGGTTGCTCTGCA
ATCCGCCATCCACTACGCCGCTGAGCGAAGACAGT
TCAACGCCACTTCGCCCACAGAGGAGGAGGTGCTC
CTGGATTACCAGCGACACCAGCGGCGCCTCTTTAC
CCGACTCGCCACCACCTACGCCGCATCGTTCGCCC
ATGAGCAACTGCTGCAGAAATTCGACGACGTGTTC
TCGGGTGCTCATGATACTGACGCCGACCGTCAGGA
CCTTGAGACACTGGCTGCTGCTCTGAAGCCCCTTT
CTACCTGGCATGCTCTCGATACCCTACAAGAGTGC
CGAGAAGCGTGTGGGGGTGCAGGTTTTCTGATTGA
GAACCGATTCGCTTCTCTCCGGGCCGATCTCGACG
TCTACGTGACCTTCGAAGGAGACAACACCGTGCTT
CTTCAGTTGGTGGCCAAGAGGCTGCTCGCTGACTA
TGCTAAGGAGTTCCGAGGTGCCAACTTCGGCGTGC
TCGCGCGGTACGTCGTGGACCAGGCTGCCGGAGTC
GCGCTACACCGAACCGGACTGCGACAGGTCGCTCA
GTTCGTGGCCGACAGTGGATCTGTCCAGAAATCTG
CTCTTGCCCTCCGAGACGAAGAAGGTCAGCGAACT
CTGCTGACCGACAGAGTCCAGTCCATGGTTGCAGA
GGTTGGCGCTGCTCTCAAAGGCGCGGGCAAGCTCC
CCCAGCACCAGGCGGCAGCACTGTTCAATCAGCAT
CAAAACGAACTGATCGAGGCTGCCCAGGCCCACGC
TGAGCTTTTACAGTGGGAGGCCTTTACTGAGGCTT
TGGCCAAGGTGGACGACGCTGGCACTAAGGAAGTG
TTGACCCGATTGCGTGACCTTTTTGGTCTGTCCCT
TATCGAGAAGCACCTCAGCTGGTATCTGATGAACG
GTAGGCTCTCGATGCAGAGAGGCCGAACGGTCGGC
ACTTACATTAATCGTCTTCTCGTTAAGATCCGACC
ACACGCACTTGATCTGGTTGATGCCTTCGGCTACG
GAGCCGAGCACCTTCGGGCCGCTATCGCCACCGGC
GCTGAGGCCACCCGACAGGACGAGGCCCGAACCTA
CTTCAGACAGCAACGAGCCTCCGGTAGCGCCCCTG
CTGACGAGAAGACACTCCTCGCTATCAAGGCCGGC
AAGTCTCGGGGACGACGAGCCAAACTGTAA
YlHXS1 Gene
(SEQ ID NO: 113)
ATGGGAAAGAATTACAAAAGTCTAGATTCTGTCGT
TGCCAGTGACTTCATCGCGTTAGGCATTACATCCG
AGGTCGCTGAGACTCTGCACGGACGGCTTGCCGAG
ATTGTGTGCAACTACGGAGCCGCTACCCCTCAGAC
TTGGATTAACATCGCCAACCACATTCTGTCGCCGG
ACCTCCCCTTCTCTTTGCACCAGATGTTATTCTAC
GGATGCTACAAGGATTTTGGCCCTGCACCTCCTGC
CTGGATTCCGGACCCCGAAAAAGTCAAGTCCACCA
ACCTAGGTGCCCTGCTGGAAAAGCGAGGAAAGGAG
TTCCTTGGTGTCAAGTACAAGGACCCCATTTCTTC
TTTTTCTCATTTCCAGGAATTTTCGGTGCGTAATC
CTGAGGTGTATTGGCGAACTGTGCTCATGGACGAG
ATGAAAATCTCCTTCAGCAAGGACCCAGAGTGTAT
CCTGCGACGAGACGACATTAACAACCCAGGAGGCT
CGGAGTGGCTTCCCGGCGGATACCTAAACTCAGCT
AAGAATTGTCTCAACGTGAACTCTAACAAGAAGTT
GAACGACACCATGATCGTGTGGCGTGACGAAGGCA
ACGACGACCTGCCCCTGAACAAGTTGACTCTGGAC
CAGCTGCGAAAGAGGGTCTGGTTGGTTGGCTACGC
CCTCGAAGAGATGGGCTTAGAGAAGGGTTGCGCTA
TTGCTATTGATATGCCCATGCACGTCGATGCTGTA
GTGATCTACCTTGCCATTGTGTTAGCCGGTTACGT
GGTCGTATCGATTGCCGATTCGTTCTCCGCTCCGG
AGATTTCCACCCGACTCAGACTTAGCAAGGCCAAG
GCAATCTTTACTCAAGATCACATCATCCGAGGTAA
GAAGAGAATCCCTCTCTATTCTCGCGTGGTTGAGG
CCAAGTCCCCAATGGCTATCGTCATACCTTGCAGC
GGATCAAACATCGGGGCTGAGCTACGGGACGGTGA
TATCTCCTGGGATTACTTCCTGGAGCGAGCCAAAG
AGTTCAAGAACTGCGAGTTTACAGCGCGTGAGCAG
CCCGTCGATGCCTACACGAACATTCTATTCTCATC
GGGCACAACGGGAGAGCCCAAGGCCATCCCCTGGA
CCCAAGCTACCCCCTTGAAAGCTGCCGCTGATGGT
TGGTCCCATCTCGACATCAGAAAAGGCGATGTGAT
CGTTTGGCCCACTAACCTGGGCTGGATGATGGGTC
CTTGGCTGGTATATGCCAGCCTACTGAACGGCGCT
TCAATCGCACTGTACAACGGATCTCCACTCGTCAG
CGGCTTTGCCAAGTTTGTTCAAGACGCCAAAGTCA
CCATGCTGGGTGTTGTTCCTTCAATCGTGCGAAGT
TGGAAGAGTACCAACTGTGTCTCTGGATACGACTG
GAGCACCATTCGATGCTTCAGTTCCTCCGGCGAGG
CTTCCAACGTTGATGAGTACCTCTGGCTTATGGGT
CGTGCGAATTACAAGCCTGTGATCGAGATGTGTGG
TGGAACAGAAATTGGTGGTGCTTTTTCGGCCGGGT
CCTTTCTTCAGGCTCAGTCTCTCTCCTCTTTCTCT
TCCCAGTGTATGGGATGCACCCTGTATATTCTCGA
CAAGAACGGTTACCCCATGCCGAAGAATAAACCCG
GTATTGGGGAGCTTGCTCTTGGCCCCGTCATGTTT
GGTGCATCGAAGACCCTCCTGAACGGAAACCATCA
CGACGTCTACTTCAAGGGCATGCCCACACTTAACG
GCGAAGTTCTTCGGCGACATGGAGACATTTTCGAA
CTTACATCGAACGGATACTACCACGCCCACGGGCG
AGCAGATGATACGATGAACATCGGGGGCATCAAGA
TATCTTCTATCGAGATTGAAAGAGTGTGTAACGAG
GTAGACGACCGCGTCTTCGAGACTACTGCGATCGG
CGTCCCCCCCCTGGGCGGTGGCCCGGAGCAGCTAG
TCATTTTTTTTGTGCTCAAGGACTCTAACGACACG
ACCATCGACCTCAATCAGCTGCGACTCTCCTTCAA
CCTTGGATTGCAGAAGAAGCTGAACCCTCTCTTCA
AGGTCACTCGGGTTGTTCCCTTGTCCTCTCTTCCT
CGAACCGCCACCAACAAGATTATGCGACGAGTGCT
CCGACAGCAGTTCTCCCACTTCGAGTAA
YlTKS1 Gene
(SEQ ID NO: 114)
ATGAATCATCTAAGAGCCGAAGGACCAGCAAGTGT
ACTCGCTATTGGTACTGCTAACCCCGAGAACATTC
TTATTCAGGATGAGTTCCCGGACTACTATTTCAGG
GTTACCAAGAGCGAGCATATGACCCAGCTTAAAGA
GAAGTTCCGCAAGATATGCGACAAGTCCATGATCC
GAAAGCGGAACTGCTTTCTCAATGAGGAGCACTTG
AAGCAAAACCCCCGACTGGTTGAGCACGAGATGCA
GACCTTGGACGCGCGACAAGACATGCTCGTCGTCG
AGGTGCCCAAACTCGGTAAGGATGCTTGCGCTAAG
GCCATTAAGGAGTGGGGTCAGCCCAAGTCGAAGAT
TACCCACCTAATCTTCACCTCCGCAAGCACTACAG
ACATGCCCGGTGCAGACTACCACTGTGCCAAGCTG
CTCGGACTGTCACCGTCGGTCAAGCGAGTGATGAT
GTACCAGCTCGGCTGTTACGGGGGTGGAACCGTTC
TCCGTATCGCCAAGGATATCGCTGAGAACAACAAA
GGAGCTCGTGTCCTGGCTGTGTGTTGCGACATCAT
GGCCTGCTTGTTCAGAGGCCCTAGTGATTCCGATC
TGGAATTACTTGTCGGTCAGGCCATCTTTGGAGAT
GGCGCCGCCGCTGTCATCGTGGGTGCCGAACCCGA
CGAGTCTGTTGGAGAAAGACCCATCTTTGAGCTTG
TCTCCACGGGCCAGACCATCCTCCCTAACAGCGAG
GGCACAATTGGAGGCCATATTCGAGAGGCCGGTCT
GATTTTTGACCTGCATAAGGACGTGCCTATGCTGA
TTTCGAACAACATCGAGAAGTGTCTCATCGAGGCC
TTCACTCCCATCGGCATTTCGGACTGGAACTCAAT
CTTCTGGATCACCCACCCAGGAGGCAAGGCGATTC
TGGATAAAGTTGAGGAAAAGCTCGACCTTAAGAAG
GAGAAGTTTGTGGATTCTCGACACGTCCTGTCTGA
ACACGGTAACATGTCTTCCTCTACTGTCCTGTTCG
TAATGGACGAGCTTCGAAAGCGATCTCTGGAGGAA
GGAAAGTCCACGACCGGCGACGGTTTTGAGTGGGG
CGTGCTGTTCGGGTTCGGTCCTGGCCTCACTGTGG
AGCGAGTTGTTGTCCGGTCCGTGCCTATTAAGTAC
TAA
YlTKS1P Gene
(SEQ ID NO: 115)
ATGAATCATCTAAGAGCCGAAGGACCAGCAAGTGT
ACTCGCTATTGGTACTGCTAACCCCGAGAACATTC
TTATTCAGGATGAGTTCCCGGACTACTATTTCAGG
GTTACCAAGAGCGAGCATATGACCCAGCTTAAAGA
GAAGTTCCGCAAGATATGCGACAAGTCCATGATCC
GAAAGCGGAACTGCTTTCTCAATGAGGAGCACTTG
AAGCAAAACCCCCGACTGGTTGAGCACGAGATGCA
GACCTTGGACGCGCGACAAGACATGCTCGTCGTCG
AGGTGCCCAAACTCGGTAAGGATGCTTGCGCTAAG
GCCATTAAGGAGTGGGGTCAGCCCAAGTCGAAGAT
TACCCACCTAATCTTCACCTCCGCAAGCACTACAG
ACATGCCCGGTGCAGACTACCACTGTGCCAAGCTG
CTCGGACTGTCACCGTCGGTCAAGCGAGTGATGAT
GTACCAGCTCGGCTGTTACGGGGGTGGAACCGTTC
TCCGTATCGCCAAGGATATCGCTGAGAACAACAAA
GGAGCTCGTGTCCTGGCTGTGTGTTGCGACATCAT
GGCCTGCTTGTTCAGAGGCCCTAGTGATTCCGATC
TGGAATTACTTGTCGGTCAGGCCATCTTTGGAGAT
GGCGCCGCCGCTGTCATCGTGGGTGCCGAACCCGA
CGAGTCTGTTGGAGAAAGACCCATCTTTGAGCTTG
TCTCCACGGGCCAGACCATCCTCCCTAACAGCGAG
GGCACAATTGGAGGCCATATTCGAGAGGCCGGTCT
GATTTTTGACCTGCATAAGGACGTGCCTATGCTGA
TTTCGAACAACATCGAGAAGTGTCTCATCGAGGCC
TTCACTCCCATCGGCATTTCGGACTGGAACTCAAT
CTTCTGGATCACCCACCCAGGAGGCAAGGCGATTC
TGGATAAAGTTGAGGAAAAGCTCGACCTTAAGAAG
GAGAAGTTTGTGGATTCTCGACACGTCCTGTCTGA
ACACGGTAACATGTCTTCCTCTACTGTCCTGTTCG
TAATGGACGAGCTTCGAAAGCGATCTCTGGAGGAA
GGAAAGTCCACGACCGGCGACGGTTTTGAGTGGGG
CGTGCTGTTCGGGTTCGGTCCTGGCCTCACTGTGG
AGCGAGTTGTTGTCCGGTCCGTGCCTATTAAGTAC
GGAAGAAGGGCAAAGTTGTAA
YlOAC1 Gene
(SEQ ID NO: 116)
ATGGCCGTCAAACACCTTATTGTCCTCAAGTTCAA
AGATGAGATCACTGAAGCCCAGAAGGAGGAGTTTT
TCAAGACCTACGTCAATTTGGTCAACATCATTCCA
GCAATGAAGGATGTGTACTGGGGCAAGGACGTGAC
CCAGAAGAACAAGGAAGAGGGTTATACCCATATCG
TTGAGGTTACGTTCGAGTCTGTGGAGACAATCCAA
GACTACATCATTCACCCCGCTCACGTGGGCTTTGG
AGACGTTTACAGATCCTTCTGGGAGAAGCTCCTGA
TTTTTGACTACACTCCTCGAAAGCTGAAGCCCAAG
TAA
YlOAC1P Gene
(SEQ ID NO: 117)
ATGGCCGTCAAACACCTTATTGTCCTCAAGTTCAA
AGATGAGATCACTGAAGCCCAGAAGGAGGAGTTTT
TCAAGACCTACGTCAATTTGGTCAACATCATTCCA
GCAATGAAGGATGTGTACTGGGGCAAGGACGTGAC
CCAGAAGAACAAGGAAGAGGGTTATACCCATATCG
TTGAGGTTACGTTCGAGTCTGTGGAGACAATCCAA
GACTACATCATTCACCCCGCTCACGTGGGCTTTGG
AGACGTTTACAGATCCTTCTGGGAGAAGCTCCTGA
TTTTTGACTACACTCCTCGAAAGCTGAAGCCCAAG
GGAAGAAGGGCAAAGTTGTAA
YlPTS2 Gene
(SEQ ID NO: 118)
ATGGGCCTCTCTCTAGTATGTACCTTCTCTTTCCA
GACCAACTATCACACTCTACTGAACCCCCATAACA
AGAACCCTAAAAATTCTCTTCTCAGTTACCAGCAC
CCCAAGACGCCTATCATTAAGTCCTCCTACGACAA
CTTTCCCTCTAAGTACTGCCTGACCAAAAACTTCC
ATCTCCTGGGACTGAACTCTCATAACAGAATTAGT
AGCCAGTCCCGATCTATCCGAGCTGGCTCTGACCA
GATTGAGGGCTCCCCTCACCATGAATCCGACAACA
GCATCGCTACCAAGATTTTGAATTTTGGTCACACA
TGCTGGAAGCTCCAGCGACCGTACGTCGTGAAGGG
TATGATCTCGATTGCCTGTGGACTGTTCGGACGTG
AGCTTTTTAATAATCGACACTTGTTTTCATGGGGC
CTCATGTGGAAGGCTTTTTTCGCCCTCGTGCCCAT
TCTGTCTTTCAACTTCTTTGCCGCTATTATGAACC
AAATCTACGACGTTGATATTGATAGGATCAACAAG
CCTGACCTGCCGCTCGTCTCGGGGGAGATGTCTAT
CGAGACAGCGTGGATTCTTTCGATTATCGTCGCGC
TGACTGGCCTTATCGTTACCATAAAGTTGAAGTCT
GCACCCCTCTTCGTGTTTATCTACATTTTCGGTAT
TTTTGCTGGATTCGCGTACTCCGTTCCCCCTATCA
GATGGAAGCAGTACCCCTTTACTAACTTTCTGATT
ACTATCAGCAGCCACGTCGGTTTAGCCTTTACCTC
ATATTCGGCCACCACCAGTGCACTGGGCCTCCCCT
TCGTCTGGCGACCTGCATTTTCATTCATCATCGCC
TTCATGACTGTGATGGGTATGACCATCGCTTTCGC
TAAGGACATCTCCGACATCGAGGGTGATGCTAAAT
ATGGAGTGTCCACCGTGGCCACTAAGCTGGGAGCC
CGGAACATGACGTTCGTCGTCTCTGGTGTTCTGCT
CCTTAACTACTTGGTTTCGATCTCCATTGGCATTA
TCTGGCCACAAGTCTTCAAGTCCAACATTATGATT
CTGTCCCACGCCATTCTTGCCTTTTGCCTGATCTT
CCAGACACGCGAACTCGCTCTCGCTAACTACGCCT
CCGCCCCATCGCGACAGTTCTTCGAGTTCATCTGG
CTGCTTTACTACGCCGAGTACTTCGTTTACGTGTT
CATCTAA
YlCBD1 Gene
(SEQ ID NO: 119)
ATGAAGTGTTCGACGTTTTCTTTTTGGTTTGTTTG
TAAAATCATTTTCTTTTTCTTTTCTTTCAACATCC
AAACGTCGATCGCAAACCCTAGAGAGAACTTTCTT
AAGTGCTTCTCGCAGTACATCCCTAATAACGCTAC
CAACCTTAAGCTGGTGTACACCCAGAACAACCCTC
TTTACATGTCTGTTCTAAACAGCACCATCCACAAT
CTTAGATTCACATCAGACACCACTCCCAAGCCGCT
CGTCATCGTGACCCCGAGTCATGTGTCCCATATCC
AAGGCACTATCCTGTGCTCTAAAAAGGTCGGTCTG
CAGATTCGGACTCGCTCCGGTGGACATGATTCGGA
GGGCATGTCCTACATTAGCCAGGTCCCCTTTGTGA
TCGTGGACCTGAGGAACATGCGGTCTATTAAGATT
GATGTGCACTCACAGACCGCTTGGGTCGAGGCTGG
TGCGACATTGGGTGAGGTGTACTACTGGGTGAACG
AGAAGAACGAGAACCTGAGCCTCGCCGCTGGCTAC
TGTCCCACCGTTTGTGCCGGTGGACACTTCGGCGG
AGGCGGATACGGTCCACTTATGCGAAACTACGGGC
TCGCAGCTGATAATATCATCGACGCACACCTTGTT
AACGTTCACGGCAAGGTGCTGGACCGAAAAAGCAT
GGGTGAGGACCTATTTTGGGCCTTGCGAGGCGGTG
GTGCCGAATCCTTCGGAATTATCGTGGCCTGGAAG
ATCCGACTGGTCGCTGTGCCAAAGTCCACTATGTT
CTCCGTCAAGAAAATTATGGAGATCCACGAACTCG
TAAAGCTCGTCAATAAGTGGCAGAACATCGCCTAC
AAGTATGACAAGGATCTGCTGCTCATGACTCACTT
CATCACGCGAAACATTACAGACAACCAGGGAAAGA
ACAAGACCGCTATCCATACCTACTTCTCCTCTGTC
TTCCTTGGGGGTGTCGATTCCCTCGTTGATCTCAT
GAACAAATCTTTTCCAGAGCTCGGAATCAAGAAGA
CCGACTGCCGACAGCTCTCTTGGATCGACACCATT
ATTTTCTACTCAGGAGTCGTAAACTACGATACTGA
CAACTTTAACAAGGAGATTCTGTTAGATCGATCGG
CCGGCCAGAACGGTGCCTTCAAGATCAAGCTCGAC
TATGTCAAAAAGCCCATTCCTGAATCCGTCTTCGT
TCAAATTCTTGAAAAGTTGTACGAGGAGGATATCG
GCGCCGGAATGTACGCGCTGTACCCCTACGGTGGC
ATTATGGACGAGATTTCTGAAAGTGCTATTCCCTT
CCCCCACCGTGCTGGCATTCTGTATGAGCTGTGGT
ACATTTGCTCCTGGGAAAAGCAGGAGGACAACGAG
AAGCACTTGAACTGGATACGAAACATTTACAATTT
CATGACCCCCTATGTTTCGAAGAACCCTCGACTGG
CCTACCTGAATTACCGCGACCTCGACATCGGAATT
AACGACCCTAAGAACCCCAATAACTATACTCAGGC
CAGAATCTGGGGCGAGAAGTACTTCGGCAAGAACT
TTGACCGTCTGGTTAAGGTCAAGACCCTCGTGGAC
CCTAACAACTTCTTCCGAAACGAGCAGTCTATCCC
CCCTCTGCCCCGACACCGGCATTAA
YlTHC1 Gene
(SEQ ID NO: 120)
ATGAATTGTTCAGCTTTCTCTTTTTGGTTTGTCTG
TAAGATCATTTTCTTCTTTCTATCCTTTCACATCC
AAATTTCCATAGCCAACCCTCGTGAGAACTTCCTT
AAGTGCTTTTCCAAGCACATTCCAAATAACGTCGC
CAATCCCAAGCTGGTGTACACGCAGCATGACCAGC
TCTACATGTCGATCCTCAATTCCACCATTCAAAAC
CTTAGATTCATTAGTGACACCACTCCCAAGCCCCT
AGTCATTGTCACCCCTTCGAACAACTCGCATATTC
AGGCAACTATTCTCTGCTCCAAGAAGGTTGGTTTA
CAGATCCGAACCCGGTCAGGTGGTCACGACGCTGA
GGGCATGTCTTACATTTCCCAGGTCCCCTTTGTGG
TGGTCGATCTGCGCAACATGCACTCCATTAAAATC
GACGTCCACTCGCAGACTGCCTGGGTCGAGGCTGG
AGCCACCCTTGGCGAGGTCTACTACTGGATTAACG
AGAAGAACGAAAACCTGTCGTTCCCTGGCGGCTAC
TGTCCGACTGTTGGAGTCGGCGGACACTTTTCTGG
TGGCGGATATGGTGCTCTCATGCGAAACTACGGAC
TGGCGGCAGACAACATCATCGATGCCCACCTTGTG
AACGTTGACGGTAAGGTACTGGACCGAAAGTCTAT
GGGCGAGGACTTGTTTTGGGCCATCCGAGGTGGAG
GTGGTGAGAACTTCGGGATCATCGCCGCCTGGAAG
ATCAAGCTGGTGGATGTGCCCAGTAAGTCTACCAT
TTTTAGCGTGAAGAAGAACATGGAGATCCACGGGC
TGGTGAAGCTGTTCAACAAGTGGCAGAATATTGCG
TACAAATACGACAAGGACCTGGTGCTTATGACCCA
TTTCATCACCAAGAACATCACGGATAACCACGGTA
AAAACAAGACTACTGTTCACGGTTACTTCTCTTCA
ATTTTCCATGGTGGTGTGGATTCCCTCGTTGATTT
GATGAACAAGTCCTTCCCAGAGCTGGGCATTAAGA
AGACAGACTGCAAGGAATTTAGCTGGATTGATACC
ACCATCTTCTACTCTGGAGTTGTCAACTTCAACAC
CGCAAACTTCAAGAAGGAAATCCTCTTGGACCGAT
CTGCCGGCAAGAAGACAGCTTTTTCGATTAAACTG
GATTACGTGAAGAAGCCCATCCCTGAGACAGCTAT
GGTCAAGATCCTTGAAAAACTTTATGAGGAGGACG
TCGGAGCCGGAATGTACGTTCTCTATCCTTACGGC
GGCATCATGGAGGAAATTTCTGAGTCTGCTATCCC
CTTCCCCCATCGAGCCGGAATCATGTACGAGCTGT
GGTACACCGCTAGTTGGGAGAAGCAGGAGGATAAC
GAGAAACATATCAATTGGGTCCGTAGCGTATACAA
TTTCACGACACCCTACGTGTCCCAGAACCCTCGAC
TCGCTTACCTGAACTATAGGGACCTGGACCTCGGC
AAGACTAACCACGCTAGCCCGAACAACTACACCCA
GGCCAGAATTTGGGGCGAAAAGTACTTCGGAAAGA
ACTTCAACCGACTCGTTAAGGTTAAGACCAAAGTT
GACCCCAACAACTTTTTCCGGAACGAGCAGTCCAT
CCCTCCACTCCCTCCCCACCATCACTGA

The following plasmids were constructed using modern molecular biology techniques described herein.

TABLE 2

| | | Important |
|---|---|---|
| Plasmid | Seq ID | Features |
| pLD87 | 166 | KU70 Disruption cassette |
| | | with LoxP-URA3-LoxP |
| pLD101 | 167 | POX5 Disruption cassette |
| | | with LoxP-URA3-LoxP |
| pLD102 | 168 | POX3 Disruption cassette |
| | | with LoxP-URA3-LoxP |
| pLD131 | 174 | CEN-ARS LEU2 PPOX2-PTS2 |
| pLD132 | 175 | CEN-ARS URA3 PPOX-CBD1 |
| pLD135 | 176 | POX3 Disruption cassette |
| | | URA3 ACO1P |
| pLD137 | 177 | CEN-ARS LEU |
| pLD138 | 178 | CEN-ARS URA |

### Example 3—Transformation of Candida viswanathii

A starting Candida viswanathii strain, such as the uracil auxotroph produced in Example 2, is propagated in a 5 mL YPD culture that is incubated overnight at 30° C., 250 rpm. The next day, a 50 mL culture is initiated with part of the 5 mL YPD overnight culture and grown for a few hours to an OD (600 nm) absorbance of 1.0-2.0. The resulting cells are pelleted by centrifugation at 1000×g for 10 minutes. The cells are washed by resuspension in sterile water, centrifuged (10000×g, 1 min) and resuspended in 1 mL sterile TE/LiOAC solution, pH 7.5. The cells were centrifuged (10000×g, 1 min) again and resuspended in 500 μl of TE/LiOAC solution and incubated with shaking at 30° C. for 30 minutes.

For each transformation reaction, 50 μl aliquots of cell suspension are to be used. To 50 μl of cells, add 5 μl of carrier DNA (boiled and cooled salmon sperm DNA, 10 mg/mL) and incubate for 1-2 minutes at room temperature. The DNA that is to be used to transform the yeast is optimally inserted into the yeast genome as linearized DNA. Therefore, 2-5 μg of linearized DNA for integration is added to the mixture of cells and salmon sperm DNA. To this mixture, 300 μl of sterile PEG solution (40% PEG 3500, 1×TE, 1×LiOAC) is added and the final mixture is incubated at 30° C. for 30-60 minutes while being gently agitated. The cells are then pelleted by centrifugation at 1000×g 30 seconds, resuspended in 500 μl of YPD media and incubated at 30° C., 250 rpm for 1-2 hours.

After recovery in YPD the cells are then pelleted by centrifugation and washed twice with 1 mL of 1×TE before plating cells on the appropriate auxotrophic or selective media to identify transformants.

### Example 4—Transformation of Yarrowia lipolytica

A starting Yarrowia lipolytica strain is propagated in 2 mL YPD culture that is incubated overnight (˜20 hrs) at 30° C., 250 rpm. the resulting cells are pelleted by centrifugation at 6000×g. The cells are washed by resuspension in 250 μl of 0.3 M Li Acetate 10 mM Tris-HCl pH 8.0. The cells are then pelleted and resuspended in 100 μl of 0.3 M Li Acetate 10 mM Tris-HCl pH 8.0. To the cells 5 μl of salmon sperm DNA solution (8 mg/ml ssDNA 10 mM Tris-HCL pH 8.0 1 mM EDTA) is added, 1 to 10 μl of DNA (up to 1 μg) and 15 μl of triacetic solution (95 μl of triacetin+5 μl beta-mercaptoethanol). Cells are mixed by pipetting and incubated 30 min at room temperature.

150 μl of PEG solution is added (40% PEG 3500, 1×TE, 1×LiOAC) and mix via pipetting. Incubate at 30 min at 30 min at room temperature. Heat shock at 37 C for 15 to 25 minutes in water batch. Add 1 ml water, mix well and then pellet at 6000×g. Decant, resuspend in 100 μl of TE (10 mM Tris-HCl pH 8.0+1 mM EDTA and plate in desired media.

### Example 5—Production of an Uracil Auxotroph of ATCC 20962

ATCC 20962 is a prototrophic yeast strain that is able to grow in the absence of supplemented uracil. In order to utilize this strain for experiments, it must first be made auxotrophic for uracil. In the case of ATCC 20962, the URA3 gene must be inactivated to make the strain auxotrophic. This will allow for uracil auxotrophy to be rescued by the introduction of a functional URA3 gene via transformation, as described in Example 3.

To convert ATCC 20962 to a uracil auxotroph, an individual colony of the strain was grown in 5 mL of YPD overnight at 30° C., shaken at 250 rpm. From the overnight culture, 20 and 100 μl of culture were plated on plates containing 5-FOA (recipe). The chemical 5-FOA is converted into a toxic compound, fluorodeoxyuridine, by the enzyme encoded by the URA3 gene. Therefore, growth on 5-FOA selects for uracil auxotrophs that have spontaneously produced loss-of-function ura3 mutants. The plate was placed at 30° C. for 3-6 days to produce colonies. The resulting colonies were tested for growth on media lacking uracil, e.g. synthetic complete yeast media lacking uracil and 5-FOA plates. One of these colonies did not grow on media lacking uracil but grew on 5-FOA plates, it was confirmed as uracil auxotrophs and named LCV32.

### Example 6—Genomic DNA Extraction for PCR Analysis

An overnight culture in YPD or selective media was grown at 30° C. 225 rpm. Cells were pelleted in a screw-cap 1.5 ml microcentrifuge tube. Supernatant was decanted and cells resuspended in 250 μl of extraction buffer (100 mM NaCl, 2% Triton X-100, 1% SDS, 10 mM Tris-Cl, 1 mM EDTA pH 8.0). 200 μl of acid washed glass-beads (425-600), and 300 μl of phenol:chorofom:isoamyl alcohol solution (25:24:1 ratio) that has been equilibrated with 100 mM Tris Cl pH 8.0 is added and vortexed for 5 min. The microcentrifuge is then centrifuge and the supernatant is removed. The supernatant then is extracted with 300 μl of chloroform. The aqueous solution is then transferred to a new microcentrifuge tube. 1.2 ml of ice-cold ethanol is added to the microcentrifuge tube. The tube is then mixed and placed at −20° C. or colder for 1 hr. The DNA is then pelleted by centrifugation at 10,000×G. The pellets are then air-dried and resuspended in 500 μl of TE (10 mM Tris-HCl 1 mM EDTA pH 8.0).

### Example 7—Verification of Integrations in Candida viswanathii

To verify the integration of exogenous genes into the genome of Candida viswanathii a PCR based method was developed where one or two of different exogenous genes were amplified in the same reaction as a section of the actin gene that serves as a control.

TABLE 3

| | Forward | Reverse | |
|---|---|---|---|
| Gene | Primer | Primer | Length |
| CvACT1 | 4 | 5 | 859 |
| CvHXS1 | 6 | 7 | 612 |
| CvTKS1 | 8 | 9 | 617 |
| CvOAC1 | 10 | 11 | 461 |
| CvPTS1 | 12 | 13 | 611 |
| CvCBD1 | 14 | 15 | 411 |
| CvACO1 | 16 | 17 | 414 |
| CvPTS2 | 41 | 42 | 616 |
| CvTHC1 | 168 | 169 | 397 |

For a 25 μl reaction, 12.5 μl of the 2× Master Mix, 0.5 μl of primers (10 μM of each), 1 μl of DNA template, and 11 μl of water. Standard running reactions are 95° C. for 2 min, 30 cycles of 95° C. for 30 sec, 55° C. for 30 sec, and 72° C. for 1 min, and a final step of 72° C. for 2 min. 10 μl to 20 μl is loaded in a 1.4 to 2.0% agarose TAE gel.

TABLE 4

| Primer | Sequence |
|---|---|
| number | |
| 4 | CCCAATTCCTGTGGTGGGTTGATTCG |
| | (SEQ ID NO: 121) |
| 5 | CTCTCAATTCGTTGTAGAAGGTGTGGTGC |
| | (SEQ ID NO: 122) |
| 6 | CTTTGGACTCAGTTGTGGCTAGTGACTTC |
| | (SEQ ID NO: 123) |
| 7 | GATCAAGAGTCAATTTATTCAATGGGAGATCGTC |
| | (SEQ ID NO: 124) |
| 8 | GAGCAGAAGGACCCGCATCAGTG |
| | (SEQ ID NO: 125) |
| 9 | GTCACCAAAAATTGCCTGTCCGACAAGC |
| | (SEQ ID NO: 126) |
| 10 | CACGACATAATGGCAGTCAAACACCTAATAG |
| | (SEQ ID NO: 127) |
| 11 | CTAGTTTTGTGTTCGGAGTATGCATACAACG |
| | (SEQ ID NO: 128) |
| 12 | CTACCACACCCTCCTAAACCCTCAC |
| | (SEQ ID NO: 129) |
| 13 | CGAAGCAGTACCCGAATATATATAATGGACC |
| | (SEQ ID NO: 130) |
| 14 | CCTTCAACATTCAGACATCAATCGCCAAC |
| | (SEQ ID NO: 131) |
| 15 | CACCCAAGGTTGCCCCTGCTTC |
| | (SEQ ID NO: 132) |
| 16 | GAGCAAGTTCCCCAGCAAGTCCAG |
| | (SEQ ID NO: 133) |
| 17 | CATGATGTTCACGGGTTCCCAAGTGC |
| | (SEQ ID NO: 134) |
| 41 | CATACATGCTGGAAGCTACAGCGAC |
| | (SEQ ID NO: 135) |
| 42 | CAGTACTCACCCCATATTTTGCATCGC |
| | (SEQ ID NO: 136) |
| 168 | GTTAAGTTGTTTAACAAGTGGCAAAACATTGCATAC |
| | (SEQ ID NO: 137) |
| 169 | CCAAAATCTTAACCATTGCTGTTTCTGGAATTGG |
| | (SEQ ID NO: 138) |
| 170 | GATTTGTTTTGGGCAATTAGAGGAGGTGG |
| | (SEQ ID NO: 139) |
| 171 | CAACTCCTGAGTAGAAAATGGTTGTATCGATC |
| | (SEQ ID NO: 140) |

### Example 8—Construction of Yeast Strains in Candida viswanathii

The following strains were constructed by transformation as described in the previous example with a PacI digested plasmid as shown in Table 5. Ura+ strains were tested for successful integration of the gene or genes of interest by using the PCR-based method described above. A correct strain was named according to Table 5.

TABLE 5

| Strain | Parental Strain | Plasmid Transformed |
|---|---|---|
| LCV13 | ATCC20913 | pLD1 |
| LCV14 | ATCC20913 | pLD1 |
| LCV22 | ATCC20913 | pLD10, pLD12, pLD16 |
| LCV34 and LCV35 | LCV32 | pLD22 |
| LCV36 | LCV32 | pLD14, pLD22, pLD24 |
| LCV38 | LCV32 | pLD10, pLD12, |
| | | pLD116, pLD22 |
| LCV40 | LCV32 | pLD1 |
| LCV49 | LCV32 | pLD14, pLD22, pLD24, |
| | | pLD26 |
| LCV50 | ATCC20913 | pLD56, pLD111 |
| LCV51 | ATCC20913 | pLD19 |
| LCV55 | ATCC20913 | pLD56 |
| LCV59 | ATCC20913 | pLD20, pLD56 |
| LCV61 | ATCC20913 | pLD56, pLD111 |
| LCV63 | ATCC20913 | pLD56, pLD126 |
| LCV67 | ATCC20913 | pLD56, pLD140 |
| LCV70 | ATCC20913 | pLD56, pLD139 |

### Example 9—Construction of Yeast Strains in Yarrowia lipolytica

MYA-2613 (ATCC) was transformed with PacI digested pLD87 and Ura+ transformants selected by growth in ScD-ura plates. Genomic DNA from the ura+ strains was purified as described above. To identify the disruption of the KU70 gene a four oligo PCR method was designed. Two primers (61+87) amplifies a 796 bp piece of the actin gene, and primers 63 and 64 amplifies a 558 bp piece that is replaced by the URA3 gene if the KU70 gene is disrupted.

Using genomic DNA as the template for the method described allowed to identify if the strain still had the KU70 gene (presence of both PCR fragments) or if the strain had a disrupted KU70 (presence of only the actin PCR fragment). One strain was identified with the correct PCR profile and named YYL2.

YYL2 was transformed with pLD113 and Leu+ transformants selected by growth in ScD-leu plates. Leu+ transformant was then streaked in ScD+FOA plates. FOA resistant strains were isolated and its genomic DNA purified. To identify loss of the URA3 a four oligo PCR method was designed. Two primers (61+87) amplifies a 796 bp piece of the actin gene, and primers 115 and 116 amplifies a 594 bp piece ku70-URA3 piece that is lost if the URA3 piece is removed from the KU70 loci.

Using genomic DNA as the template for the method described allowed to identify if the strain still had the URA3 at the KU70 loci (presence of both PCR fragments) or if the strain had lost the URA3 at the KU70 loci (presence of only the actin PCR fragment). One strain was identified with the correct PCR profile and named YYL4.

TABLE 6

| | Parental | Plasmid |
|---|---|---|
| Strain | Strain | Transform |
| YYL2 | MYA-2613 | Pac1 (pLD87) |
| YYL2L | YYL2 | pLD113 |
| YYL4 | YYL2L | |
| YYL6 | YYL4 | pLD101 |
| YYL27 | YYL25 | pLD102 |
| YYL29 | YYL25 | pLD135 |

TABLE 7

| Strain | Background | Plasmid |
|---|---|---|
| YYL7 | MYA-2613 | pLD131 |
| YYL9 | MYA-2613 | pLD132 |
| YYL11 | MYA-2613 | pLD137 |
| YYL13 | MYA-2613 | pLD138 |
| YYL17 | MYA-2613 | pLD131, pLD132 |
| YYL19 | MYA-2613 | pLD131, pLD138 |
| YYL21 | MYA-2613 | pLD137, pLD138 |

TABLE 8

| Primer | |
|---|---|
| number | Sequence |
| 61 | TTGTTACCAACTGGGATGACATGGAGAAG |
| | (SEQ ID NO: 141) |
| 63 | CTGATGGACGTGTTTTTCGACATGAACC |
| | (SEQ ID NO: 142) |
| 64 | GAAAGGAACATAGTCATTTCCAAACTTGAAAGTC |
| | (SEQ ID NO: 143) |
| 87 | CAGACGGAGTACTTTCGCTCGAGG |
| | (SEQ ID NO: 144) |
| 115 | CCCAAATTTAGCTGCATCATTCATCAACC |
| | (SEQ ID NO: 145) |
| 116 | CCGTGCTTAAGAGCAAGTTCCTTGAGG |
| | (SEQ ID NO: 146) |
| 177 | CTACGACATGCCCAAGGAGCAGC |
| | (SEQ ID NO: 147) |
| 178 | GGATTCGCACATTGGTGAACTGGATC |
| | (SEQ ID NO: 148) |
| 179 | CCAAGCGACGACAAGCTGTTGAGC |
| | (SEQ ID NO: 149) |
| 180 | CGTGTGGGTAGCAGAGTGGGC |
| | (SEQ ID NO: 150) |
| 181 | CTTTGCCATGACTGAGACTGGCCATG |
| | (SEQ ID NO: 151) |
| 182 | GAGGCGCCGCTGGTGTCG |
| | (SEQ ID NO: 152) |
| 183 | TCCGAAAGCGGAACTGCTTTCTCAATG |
| | (SEQ ID NO: 153) |
| 184 | CAGACCGGCCTCTCGAATATGGC |
| | (SEQ ID NO: 154) |
| 185 | CTAGACTACACGGGCAACCTTAACCC |
| | (SEQ ID NO: 155) |
| 186 | CTTGGGCTTCAGCTTTCGAGGAGTG |
| | (SEQ ID NO: 156) |

YYL4 was transformed with PacI digested pLD101 and Ura+ transformants selected by growth in ScD-ura plates. Genomic DNA from the ura+ strains was purified as described above. To identify the disruption of the POX5 gene a four oligo PCR method was designed. Two primers (61+87) amplifies a 796 bp piece of the actin gene, and primers 179 and 180 amplifies a 395 bp POX5 segment that is replaced by the URA3 gene.

Using genomic DNA as the template for the method described allowed to identify if the strain still had the PO5 gene (presence of both PCR fragments) or if the strain had been disrupted for POX5 (presence of only the actin PCR fragment). One strain was identified with the correct PCR profile and named YYL6.

YYL6 is transformed with pLD113 and Leu+ transformants are selected by growth in ScD-leu plates. Leu+ transformant was then streaked in ScD+FOA plates. FOA resistant strains will be isolated and its genomic DNA purified. To identify loss of the URA3 a four oligo PCR method was designed. Two primers (61+87) amplifies a 796 bp piece of the actin gene, and primers 116 and 237 amplifies a 598 bp piecePOX5-URA3 piece that is lost if the URA3 piece is removed from the URA loci. A strain with the correct PCR products was named YYL0025.

YYL25 is transformed with Pac digested pLD102 or pLD135, and Ura+ transformants selected by growth in ScD-ura plates. Genomic DNA from the ura+ strains is purified as described above. To identify the disruption of the POX4 gene a four oligo PCR method was designed. Two primers (61+87) amplifies a 796 bp piece of the actin gene, and primers 177 and 178 amplifies a 596 bp POX3 segment that is replaced by the URA3 gene. One strain transformed with either pLD102 or pLD135 is identified with the correct PCR profile and named YYL27 and YYL29, respectively.

Strain with plasmids were either transformed once (if containing one plasmid) or sequentially (if containing more than one plasmid).

### Example 10—Quantification of Olivetolic Acid and Cannabinoids

To a 100 to 500 mg wet yeast pellet 50 μl of HCl was added and vortexed for 30 seconds. 400 μl of dichloromethane: ethyl ether (1:2) was added and vortexed for 1 min and centrifuged for 2 min at 10 k rpm. The top layer was removed, and the pellet was reextracted. The top layers were combined and dried under vacuum. The powder was reconstituted in 200 μl of acetonitrile and sonicated for 1 min. The solution was centrifuge for 2 min at 10 k rpm. Solution was then used for LC MS MS analysis.

For 0.5 ml of supernatant, 0.5 ml 1 M HCl was added and vortexed for 30s. 500 μl of dichloromethane was added, vortexed for 30 sec and centrifuged for 2 min at 10 k rpm. The bottom layer was removed and the aqueous layer re-extracted. Both organic layer samples were combined and dried under vacuum. The powder was reconstituted in 200 μl of acetonitrile and sonicated for 1 min. The solution was centrifuge for 2 min at 10 k rpm. Solution was then used for LC MS MS analysis.

### Example 11—Quantification of Hexanoic Acid and Fatty Acids

To 1 ml of supernatant, 0.8 ml of 6N HCl was added. 400 μl of dichloromethane: ethyl ether (1:2) was added, vortexed for 2 min and centrifuge for 2 min. The top layer was removed the extraction was repeated. The top layers were combined and dried under vacuum. The powder was reconstituted in 167 μl acetonitrile and 33 μl of acetonitrile with 20 mg/ml isopropyl alcohol and sonicated for 1 min. The solution was centrifuged for 2m in at 10 k rpm. Solution was then used for GC FID analysis.

### Example 12—Yeast Media

### ScD-ura

1 L of liquid media was made by making a 100 ml solution of 20% dextrose and a 900 ml solution with 1.7 g of Yeast Nitrogen Base without ammonium sulfate and amino acids, 5 g of ammonium sulfate, and 2 g of dropout amino acid mix without uracil (Sunrise Science Products). Both solutions were combined, and filter sterilized or were autoclaved separately and combined.

1 L worth of plates (˜40 plates) was made by making a 100 ml solution of 20% dextrose, a 450 ml solution with 1.7 g of Yeast Nitrogen Base without ammonium sulfate and amino acids, 5 g of ammonium sulfate, and 2 g of amino acid mix without uracil, and a 450 ml solution with 20 g of agar. The solutions were autoclaved separately and combined.

### ScD-leu

1 L of liquid media was made by making a 100 ml solution of 20% dextrose and a 900 ml solution with 1.7 g of Yeast Nitrogen Base without ammonium sulfate and amino acids, 5 g of ammonium sulfate, and 2 g of dropout amino acid mix without leucine (Sunrise Science Products). Both solutions were combined, and filter sterilized or were autoclaved separately and combined.

1 L worth of plates (˜40 plates) was made by making a 100 ml solution of 20% dextrose, a 450 ml solution with 1.7 g of Yeast Nitrogen Base without ammonium sulfate and amino acids, 5 g of ammonium sulfate, and 2 g of amino acid mix without leucine and a 450 ml solution with 20 g of agar. Both solutions were combined, and filter sterilized or autoclaved separately and combined.

### ScD-ura-leu

1 L of liquid media was made by making a 100 ml solution of 20% dextrose and a 900 ml solution with 1.7 1.7 g of Yeast Nitrogen Base without ammonium sulfate and amino acids, 5 g of ammonium sulfate, and 2 g of dropout amino acid mix without leucine and uracil (Sunrise Science Products). Both solutions were combined, and filter sterilized or autoclaved separately and combined.

1 L worth of plates (˜40 plates) was made by making a 100 ml solution of 20% dextrose, a 450 ml solution with 1.7 g of Yeast Nitrogen Base without ammonium sulfate and amino acids, 5 g of ammonium sulfate, and 2 g of amino acid mix without leucine and uracil, and a 450 ml solution with 20 g of agar were made. The solutions were autoclaved separately and combined.

ScG-ura, ScG-leu, ScG-ura-leu was made the same as ScD-ura, ScD-leu or ScD-ura-leu respectively except the dextrose was replaced with glycerol at a 40 g/L concentration. ScGP-ura, ScGP-leu, ScGP-ura-leu was the same is the same as ScG-ura, ScG-leu or ScG-ura-leu, except, monopotassium and dipotassium phosphate was added to a final concentration of 1 g/L.

### YPD

1 L of liquid media was made by making a 100 ml solution of 20% dextrose and a 900 ml solution with 10 g yeast extract and 20 g of peptone. Both solutions were combined, and filter sterilized or autoclaved separately and combined.

1 L worth of plates (˜40 plates) was made by making a 100 ml solution of 20% dextrose, a 450 ml solution with 10 g yeast extract and 20 g of peptone, and a 450 ml solution with 20 g of agar. The solutions were autoclaved separately and combined afterwards.

### SD+FOA

1 L worth of plates (˜40 plates) was made by making a 100 ml solution of 20% dextrose, a 450 ml solution with 1.7 g of yeast nitrogen base without ammonium sulfate and amino acids, 5 g of ammonium sulfate, and 2 g of amino acid mix without uracil, 0.5 g 5-FOA, 250 mg uracil, and a 450 ml solution with 20 g of agar. The solutions were autoclaved separately and combined.

### SmP

1 L of liquid media was made by dissolving 1.7 g of yeast nitrogen base without ammonium sulfate and amino acids, 5 g of ammonium sulfate, 1 g potassium phosphate, monobasic and 1 g potassium phosphate, bibasic. The solution was then filter sterilized.

### YNEP Media

1 L of YNEP was made by dissolving 3 g yeast extract, 1.7 g of yeast nitrogen base without ammonium sulfate and amino acids, 5 g of ammonium sulfate, 1 g potassium phosphate, monobasic and 1 g/l of dextrose or glycerol per liter. The solution was then filter sterilized.

### MLM Media

1 L of MLM media was made by dissolving 3 g yeast extract,1.7 g of yeast nitrogen base without ammonium sulfate and amino acids, 1 g potassium phosphate, monobasic and 1 g/l of dextrose or glycerol. Filter sterilized.

ScP-ura, ScP-leu, ScP-ura-leu was the same as Sc-ura, Sc-leu, Sc-ura-leu, respectively except monopotassium and dipotassium phosphate was added to a final concentration of 1 g/L.

For Yarrowia media a supplement of thiamine, biotin and Myo-inositol can be added to the media to a final concentration of 300 μg/L, 8 μg/L and 4 μg/L.

### Example 13—Shake Flask Fermentation Candida

A shake flask fermentation is a small-scale culture to test for production of a cannabinoid or cannabinoid precursor. A yeast strain to be tested is grown on ScD-ura, or YPD for 2-3 days. Individual colonies from those plates are propagated overnight in 5 mL of YPD or ScD-ura (30° C., shaking at 250 rpm). The resulting culture is propagated overnight in 50 mL of YPD or 25 ml of YNEP media (30° C., shaking at 250 rpm). The biomass from the 50 mL culture is spun down and washed with water, then resuspended in 15 mL of 1×SMP or MLM media with 300 μl of oleic acid (technical grade, 90%). Yeast cells, media and oleic acid are placed into a 250 mL culture flask (it may be a baffled flask for increased aeration of the culture) and incubated at 30° C. while shaking (250 rpm) for a 48-72 hours. The contents of the shake flask are spun down to produce a cell pellet and a supernatant, which is the culture media. The cell pellet and/or supernatant are analyzed for their contents by the appropriate method(s) as needed for the analyte(s) of interest.

### Example 14—Shake Flask Fermentation Yarrowia

A shake flask fermentation is a small-scale culture to test for production of a cannabinoid or cannabinoid precursor. A yeast strain to be tested is grown on ScD-ura, ScD-leu, ScD-ura-leu or YPD for 2-3 days. Individual colonies from those plates are propagated overnight in 5 mL of YPD, ScD-ura, ScD-leu, or ScD-ura-leu (30° C., shaking at 250 rpm). The resulting culture is propagated overnight in 25 mL ScGP, ScGP-ura, ScGP-leu, or ScGP-ura-leu (30° C., shaking at 250 rpm). The biomass from the 25 mL culture is spun down and washed with water, then resuspended in 20 mL of ScP, ScP-ura, ScP-leu or ScP-ura-leu media with 800 μl of oleic acid (technical grade, 90%). Yeast cells, media and oleic acid are placed into a 250 mL culture flask (it may be a baffled flask for increased aeration of the culture) and incubated at 30° C. while shaking (250 rpm) for a 24-72 hours. The contents of the shake flask are spun down to produce a cell pellet and a supernatant, which is the culture media. The cell pellet and/or supernatant are analyzed for their contents by the appropriate method(s) as needed for the analyte(s) of interest.

### Example 15—Production of Olivetolic Acid from Hexanoic Acid or Hexanoate-Esters in Candida

Strain LCV22 (HXS1 TKS1 OAC) was grown overnight in 25 ml of YPD in a 250 ml flask at 225 rpm and 30° C. 3 ml of the overnight culture was used to inoculate 50 ml of YPD and grown overnight in a 250 ml flask at 225 rpm and 30° C. A total of seven flasks were started. The biomass from the 50 mL culture was spun down and washed with water, then resuspended in 15 mL of 1×SMP media with 300 μl of oleic acid (technical grade, 90%). Yeast cells, media and oleic acid are placed into a baffled 250 mL culture flask. Different amounts of hexanoic acid, ethyl hexanoate, and geranyl hexanoate were added to different flask as described in Table 9. and incubated at room temperature while shaking (250 rpm) for 72 hours. The contents of the shake flask were spun down to produce a cell pellet and a supernatant, which is the culture media. The cell pellets were analyzed for olivetolic acid using an LC-MS MS method.

As shown in Table 9, there was an increase in olivetolic production using hexanoate ester as opposed to hexanoic acid. This is consistent with hexanoic acid being more toxic per mole than geranyl hexanoate and ethyl hexanoate.

TABLE 9

| | | Amount | Olivetolic acid |
|---|---|---|---|
| Flask | Addition | (ul) | (Area per mg) |
| 1 | — | — | 5 |
| 2 | Hexanoic acid | 15 | 13 |
| 3 | Ethyl hexanoate | 20 | 56 |
| 4 | Geranyl hexanoate | 34 | 179 |
| 5 | Hexanoic acid | 30 | 101 |
| 6 | Ethyl hexanoate | 40 | 62 |
| 7 | Geranyl hexanoate | 68 | 344 |

### Example 16—Production of Hexanoic Acid from Oleic Acid in Candida

Strain LCV14 (wildtype) and LCV35 (Δpox+ACO1P) were grown in 3 ml of YPD. These overnight cultures were used to inoculate 50 ml of YPD and grown overnight in a 250 ml flask at 225 rpm and 30° C. The biomass from the 50 mL culture was spun down and washed with water, then resuspended in 15 mL of 1×SMP media with 300 μl of oleic acid (technical grade, 90%). Yeast cells, media and oleic acid were placed into a 250 mL culture flask and incubated at 30° C. while shaking (250 rpm) for 48 hours. The contents of the shake flask were spun down to produce a cell pellet and a supernatant, which is the culture media. The cell pellets were analyzed for hexanoic using an LC-MS MS method. As shown in Table 10, LCV35 produced more hexanoic acid than LCV35, which is consistent with the presence of an active pathway making hexanoic acid from oleic acid.

TABLE 10

| | Hexanoic Acid |
|---|---|
| Strain | (mg/L) |
| LCV14 | 9.7 |
| LCV35 | 61.2 |

### Example 17—Production of Hexanoic Acid from Soybean Oil in Candida

Strain LCV14 (wildtype) and LCV34 (Δpox+ACO1P) were grown in 3 ml of YPD. These overnight cultures were used to inoculate 50 ml of YPD and grown overnight in a 250 ml flask at 225 rpm and 30° C. The biomass from the 50 mL culture was spun down and washed with water, then resuspended in 15 mL of 1×SMP media with 300 μl of soybean oil. Yeast cells, media and soybean oil were placed into a 250 mL culture flask and incubated at 30° C. while shaking (250 rpm) for 48 hours. The contents of the shake flask were spun down to produce a cell pellet and a supernatant, which is the culture media. The cell pellets were analyzed for hexanoic acid using an LC-MS MS method. As shown in Table 11, LCV34 produced more hexanoic acid than LCV14 when using soybean and palm oil, consistent with the presence of an active pathway making hexanoic acid from soybean oil and palm oil.

### Example 18—Production of Hexanoic Acid from Alkane in Candida

Strain LCV14 (wildtype) and LCV34 (Δpox+ACO1P) were grown in 3 ml of YPD. These overnight cultures were used to inoculate 50 ml of YPD and grown overnight in a 250 ml flask at 225 rpm and 30° C. The biomass from the 50 mL culture was spun down and washed with water, then resuspended in 15 mL of 1×SMP media with 300 μl of hexadecane or octadecane. Yeast cells, media and alkane were placed into a 250 mL culture flask and incubated at 30° C. while shaking (250 rpm) for 48 hours. The contents of the shake flask were spun down to produce a cell pellet and a supernatant, which is the culture media. The cell pellets were analyzed for hexanoic acid using an LC-MS MS method. As shown in Table 11, LCV34 produced more hexanoic acid than LCV14 from hexadecane, which is consistent with the presence of an active pathway making hexanoic acid from hexadecane. Octadecane may not be utilized effectively by the yeast strains.

TABLE 11

| | | Hexanoic Acid |
|---|---|---|
| Strain | Substrate | (mg/L) |
| LCV14 | Soybean Oil | 70 |
| LCV34 | Soybean Oil | 190 |
| LCV14 | Palm Oil | 47 |
| LCV34 | Palm Oil | 222 |
| LCV14 | Octadecane | 10 |
| LCV34 | Octadecane | 3 |
| LCV14 | Hexadecane | 2 |
| LCV34 | Hexadecane | 174 |

### Example 19—Production of Olivetolic Acid from Oleic Acid in Candida

Strain LCV14 (wildtype), LCV36 (Δpox+ACO1P+TKS1P+OAC1P), and LCV38 (Δpox+ACO1P+HKS1+TKS1+OAC1) were grown in 3 ml of YPD. These overnight cultures were used to inoculate 50 ml of YPD and grown overnight in a 250 ml flask at 225 rpm and 30° C. The biomass from the 50 mL culture was spun down and washed with water, then resuspended in 15 mL of 1×SMP media with 300 μl of oleic acid (technical grade, 90%). Yeast cells, media and oleic acid were placed into a 250 mL culture flask and incubated at 30° C. while shaking (250 rpm) for 48 hours. The contents of the shake flask were spun down to produce a cell pellet and a supernatant, which is the culture media. The supernatants were analyzed for olivetolic acid using an LC-MS MS method. As shown in Table 12, LCV36 and LCV38 produced more olivetolic acid than LCV14, which is consistent with the presence of an active pathway making olivetolic acid from oleic acid with either a peroxisomal TKS1P and OAC1P or a non-peroxisomal HKS1, TKS1 and OAC1.

TABLE 12

| Strain | Olivetolic Acid (Area/g) |
|---|---|
| LCV14 | 611 |
| LCV36 | 1094 |
| LCV38 | 1026 |

### Example 20—Production of Olivetolic Acid from Soybean Oil and Palm Oil in Candida

Strain LCV14 (wildtype) and LCV36 (Δpox+ACO1P+TKS1P+OAC1P) are grown in 3 ml of YPD. These overnights are used to inoculate 50 ml of YPD and grown overnight in a 250 ml flask at 225 rpm and 30° C. The biomass from the 50 mL culture is spun down and washed with water. It is resuspended in 15 mL of 1×SMP media with 300 μl of soybean oil or 300 μl of palm oil. Yeast cells, media and soybean oil are placed into a 250 mL culture flask. and incubated at 30° C. while shaking (250 rpm) for 48 hours. The contents of the shake flask str spun down to produce a cell pellet and a supernatant, which is the culture media. The cell pellets and supernatants are analyzed for olivetolic acid using an LC-MS MS method.

### Example 21—Production of Olivetolic Acid from Alkanes in Candida

Strain LCV14 (wildtype) and LCV36 (Δpox+ACO1P+TKS1P+OAC1P) are grown in 3 ml of YPD. These overnights are used to inoculate 50 ml of YPD and grown overnight in a 250 ml flask at 225 rpm and 30° C. The biomass from the 50 mL culture is spun down and washed with water. It is resuspended in 15 mL of 1×SMP media with 300 μl of hexadecane or octadecane. Yeast cells, media and alkane is placed into a 250 mL culture flask, and incubated at 30° C. while shaking (250 rpm) for 48 hours. The contents of the shake flask are spun down to produce a cell pellet and a supernatant, which is the culture media. The cell pellets and supernatant are analyzed for olivetolic acid using an LC-MS MS method.

### Example 22—Production of CBGA from Olivetolic Acid in Candida

Strain LCV51 (PTS1), LCV55 (PTS2) and LCV13 (wildtype) were grown in 3 ml of YPD. These overnight cultures were used to inoculate 2× (for LCV55) or 1× (for LCV13 and LCV5150 ml cultures of YPD and grown overnight in a 250 ml flask at 225 rpm and 30° C. The biomass from the 50 mL cultures were spun down and washed with water, then resuspended in 15 mL of 1×SMP media with 300 μl of oleic acid. Yeast cells, media and oleic acid were placed into a 250 mL culture flask. To one of the LCV55 flasks and the LCV13 flask, olivetolic acid was added to a final concentration of 1 mM. The flasks were then incubated at 30° C. while shaking (250 rpm) for 48 hours. The contents of the shake flask were spun down to produce a cell pellet and a supernatant, which is the culture media. The cell supernatants were analyzed for CBGA using an LC-MS MS method. As shown in Table 13, LCV51 and LCV55 produced more CBGA than LCV13 and LCV55 without olivetolic acid, which is consistent with the presence of an active pathway making CBGA acid from oleic acid when either PTS1 or PTS2 is expressed.

TABLE 13

| StrainStrain | | CBGA Area/ul |
|---|---|---|
| LCV13 | Yes | 18,200 |
| LCV51 | Yes | 21,920 |
| LCV55 | No | 0 |
| LCV55 | Yes | 36,000 |

### Example 23—Production of CBDA from Olivetolic Acid in Candida

Strain LCV55 (PTS2) and LCV59 (PTS2 CBD1) is grown in 3 ml of YPD. These overnights are used to inoculate 2× (for LCV59) or 1× (for LCV55) 50 ml of YPD and grown overnight in a 250 ml flask at 225 rpm and 30° C. The biomass from the 50 mL culture is spun down and washed with water, then resuspended in 15 mL of 1×SMP media with 300 μl of oleic acid. Yeast cells, media and oleic acid are placed into a 250 mL culture flask. To one of the LCV59 flasks and the LCV55 flask, olivetolic acid is added to a final concentration of 1 mM. The flasks are then incubated at 30° C. while shaking (250 rpm) for 48 hours. The contents of the shake flask are spun down to produce a cell pellet and a supernatant, which is the culture media. The cell pellets and supernatants are analyzed for CBDA using an LC-MS MS method.

### Example 24—Production of THCA from Olivetolic Acid in Candida

Strain LCV55 (PTS2) and LC61 (PTS2 THC1) are grown in 3 ml of YPD. These overnight cultures are used to inoculate 2× (for LCV61) or 1× (for LCV155) 50 ml of YPD and grown overnight in a 250 ml flask at 225 rpm and 30° C. The biomass from the 50 mL culture is spun down and washed with water, then resuspended in 15 mL of 1×SMP media with 300 μl of oleic acid. Yeast cells, media and oleic acid are placed into a 250 mL culture flask. To one of the LCV61 flasks and the LCV55 flask, olivetolic acid is added to a final concentration of 1 mM. The flasks are then incubated at 30° C. while shaking (250 rpm) for 48 hours. The contents of the shake flask are spun down to produce a cell pellet and a supernatant, which is the culture media. The cell pellets and supernatants are analyzed for THCA using an LC-MS MS method.

### Example 25—Production of CBGA from Fatty Acids in Candida

Strain LCV36 (Δpox+ACO1P+TKS1P+OAC1P) and LCV49 (Δpox+ACO1P+TKS1P+OAC1P+PTS1dN), were grown in 3 ml of YPD. These overnight cultures were used to inoculate 50 ml of YPD and grown overnight in a 250 ml flask at 225 rpm and 30° C. The biomass from the 50 mL culture was spun down and washed with water, then resuspended in 15 mL of 1×SMP media with 300 μl of oleic acid (technical grade, 90%). Yeast cells, media and oleic acid were placed into a 250 mL culture flask and incubated at 30° C. while shaking (250 rpm) for 48 hours. The contents of the shake flask were spun down to produce a cell pellet and a supernatant, which is the culture media. The supernatants were analyzed for CBGA using an LC-MS MS method. As shown in Table, LCV49 produced more CBGA than LCV36, which is consistent with the presence of an active pathway making CBGA from oleic acid.

TABLE 14

| Strain | CBGA Area/ul |
|---|---|
| LCV36 | 1,058 |
| LCV49 | 2,000 |

### Example 26—Production of CBDA from Fatty Acids in Candida

A strain is created that expresses ACO1P, TKS1P, AOC1P, PTS2 and CBD1 in a pox deleted background. This strain is grown overnight in 3 mol of YPD. This overnight culture is used to inoculate 50 ml of YPD and grown overnight in a 250 ml flask at 225 rpm and 30° C. The biomass from the 50 mL culture is spun down and washed with water. It is resuspended in 15 mL of 1×SMP media supplemented with 300 μl of oleic acid. Yeast cells, media and oleic are placed into a 250 mL culture flask. The flasks are then incubated at 30° C. while shaking (250 rpm) for 48 hours. The contents of the shake flask are spun down to produce a cell pellet and a supernatant. The cell pellet and supernatants are analyzed for CBDA acid using an LC-MS MS method.

### Example 27—Production of THCA from Fatty Acids in Candida

A strain is created that expresses ACO1P, TKS1P, AOC1P, PTS2 and THC1 or ACO1, HXS1, TKS1, AOC1, PTS2, and THC1 in a pox deleted background. This strain is grown overnight in 3 mol of YPD. This overnight is used to inoculate 50 ml of YPD and grown overnight in a 250 ml flask at 225 rpm and 30° C. The biomass from the 50 mL culture is spun down and washed with water. It is resuspended in 15 mL of 1×SMP media with 300 μl of oleic acid. Yeast cells, media and oleic acid are placed into a 250 mL culture flask. The flask is then incubated at 30° C. while shaking (250 rpm) for 48 hours. The contents of the shake flask are spun down to produce a cell pellet and a supernatant, which is the culture media. The cell pellet and supernatant is analyzed for THCA acid using an LC-MS MS method.

### Example 28—Enhanced Secretion of CBDA in Candida

Strain LCV59 (PTS2 CBD1), LCV63 (PTS2 CBD1dNS1), LCV67 (PTS2 CBDdNV1), and LCV70 (PTS2 CBD1dNP1) are grown in 3 ml of ScD-ura. These overnight cultures are used to inoculate 50 ml of YPD and grown overnight in 1 250 ml flask at 225 rpm and 30° C. The biomass from the 50 mL culture is spun down and washed with water, then resuspended in 15 mL of 1×SMP media with 300 μl of oleic acid. Yeast cells, media and oleic acid is placed into a 250 mL culture flask. Olivetolic acid is added to a final concentration of 1 mM. The flasks are then incubated at 30° C. while shaking (250 rpm) for 48 hours. The contents of the shake flask are spun down to produce a cell pellet and a supernatant, which is the culture media. The cell pellets and supernatants are analyzed for CBDA using an LC-MS MS method.

### Example 29—Enhanced Secretion of THCA in Candida

Strains expressing either (PTS2 THC1), (PTS2 THC1dNS1), (PTS2 THCdNV1), or (PTS2 THC1dNP1) were grown in 3 ml of ScD-ura. These overnight cultures are used to inoculate 50 ml of YPD and grown overnight in a 250 ml flask at 225 rpm and 30° C. The biomass from the 50 mL culture is spun down and washed with water, then resuspended in 15 mL of 1×SMP media with 300 μl of oleic acid. Yeast cells, media and oleic acid is placed into a 250 mL culture flask. Olivetolic acid is added to a final concentration of 1 mM. The flasks are then incubated at 30° C. while shaking (250 rpm) for 48 hours. The contents of the shake flask are spun down to produce a cell pellet and a supernatant, which is the culture media. The cell pellets and supernatants are analyzed for THCA using an LC-MS MS method.

### Example 30—Production of CBCA in Candida from Fatty Acids

A strain is created that expresses ACO1P, TKS1P, OAC1P, PTS2 and CBC1 or ACO1P, HXS1, TKS1, OAC1, PTS2, and CBC1 in a pox deleted background. This strain is grown overnight in 3 mol of YPD. This overnight culture is used to inoculate 50 ml of YPD and grown overnight in a 250 ml flask at 225 rpm and 30° C. The biomass from the 50 mL culture is spun down and washed with water, then resuspended in 15 mL of 1×SMP media with 300 μl of oleic acid. Yeast cells, media and oleic acid are placed into a 250 mL culture flask. The flask is then incubated at 30° C. while shaking (250 rpm) for 48 hours. The contents of the shake flask are spun down to produce a cell pellet and a supernatant, which is the culture media. The cell pellet and supernatant are analyzed for CBCA acid using an LC-MS MS method.

### Example 31—Production of Hexanoic Acid from Fatty Acids in Yarrowia

Strain YYL2 (wildtype), YYL6 (Δpox5), YYL27(Δpox3 Δpox5), and YYL29(Δpox3 Δpox5+ACO1P) are grown in 3 ml of YPD. These overnight cultures are used to inoculate 25 ml of ScGP and grown overnight in a 250 ml flask at 225 rpm and 30° C. The biomass from the 25 mL culture was spun down and washed with water, then resuspended in 20 mL of ScP media with 800 μl of oleic acid (technical grade, 90%). Yeast cells, media and oleic acid were placed into a 250 mL culture flask and incubated at 30° C. while shaking (250 rpm) for 48 hours. The fermentation is analyzed for hexanoic acid using a GC-FID method.

### Example 32—Production of Hexanoic Acid from Soybean Oil and Palm Oil in Yarrowia

Strain YYL2 (wildtype), YYL6 (Δpox5), YYL27(Δpox3 Δpox5), and YYL29(Δpox3 Δpox5+ACO1P) are grown in 3 ml of YPD. These overnight cultures are used to inoculate 25 ml of ScGP and grown overnight in a 250 ml flask at 225 rpm and 30° C. The biomass from the 25 mL culture was spun down and washed with water, then resuspended in 20 mL of ScP media with 800 μl of oleic acid (technical grade, 90%). Yeast cells, media and vegetable oil were placed into a 250 mL culture flask and incubated at 30° C. while shaking (250 rpm) for 48 hours. The fermentation is analyzed for hexanoic acid using a GC-FID method.

### Example 33—Production of Hexanoic Acid from Alkanes in Yarrowia

Strain YYL2 (wildtype), YYL6 (Δpox5), YYL27(Δpox3 Δpox5), and YYL29(Δpox3 Δpox5+ACO1P) are grown in 3 ml of YPD. These overnight cultures are used to inoculate 25 ml of ScGP and grown overnight in a 250 ml flask at 225 rpm and 30° C. The biomass from the 25 mL culture is spun down and washed with water, then resuspended in 20 mL of ScP media with 800 μl of either hexadecane and octadecane. Yeast cells, media and oleic acid are placed into a 250 mL culture flask and incubated at 30° C. while shaking (250 rpm) for 48 hours. The fermentation is analyzed for hexanoic acid using a GC-FID method.

### Example 34—Production of Olivetolic Acid from Hexanoic Acid or Hexanoate-Esters in Yarrowia

A strain is created that expresses HXS1, TKS1 and OAC1 is grown overnight in 3 ml of YPD. This overnight culture is used to inoculate 25 ml of ScGP and grown overnight in a 250 ml flask at 225 rpm and 30° C. The biomass from the 25 mL culture is spun down and washed with water, then resuspended in 20 mL of ScP media with 800 μl oleic acid. Yeast cells, media and oleic acid is placed into a 250 mL culture flask. Hexanoic acid, ethyl-hexanoate, geranyl hexanoate or other hexanoate ester is added to the flask. The culture flask is incubated at 30° C. while shaking (250 rpm) for 48 hours. The contents of the shake flask are spun down to produce a cell pellet and a supernatant. The cell pellets and supernatants are analyzed for olivetolic acid using an LC-MS MS method.

### Example 35—Production of Olivetolic Acid from Fatty Acids in Yarrowia

A strain that produces hexanoic acid that expresses TKS1P and OAC1P or HXS1 TKS1 OAC1 is grown overnight in 3 ml of YPD. This overnight culture is used to inoculate 25 ml of ScGP and grown overnight in a 250 ml flask at 225 rpm and 30° C. The biomass from the 25 mL culture is spun down and is washed with water. It is resuspended in 20 mL of ScP media with 800 μl oleic acid. Yeast cells, media and oleic acid is placed into a 250 mL culture flask. The culture flask is incubated at 30° C. while shaking (250 rpm) for 48 hours. The contents of the shake flask are spun down to produce a cell pellet and a supernatant, which is the culture media. The cell pellets and supernatants are analyzed for olivetolic acid using an LC-MS MS method.

### Example 36—Production of Olivetolic Acid 1 from Soybean Oil or Palm Oil in Yarrowia

A strain that produces hexanoic acid that expresses TKS1P and OAC1P or HXS1 TKS1 OAC1 is grown overnight in 3 ml of YPD. This overnight culture is used to inoculate 25 ml of ScGP and grown overnight in a 250 ml flask at 225 rpm and 30° C. The biomass from the 25 mL culture is spun down and is washed with water, then resuspended in 20 mL of ScP media with 800 μl soybean or palm oil. Yeast cells, media and vegetable oil is placed into a 250 mL culture flask. The culture flask is incubated at 30° C. while shaking (250 rpm) for 48 hours. The contents of the shake flask are spun down to produce a cell pellet and a supernatant, which is the culture media. The cell pellets and supernatants are analyzed for olivetolic acid using an LC MS MS method.

### Example 37—Production of Olivetolic Acid from Alkanes in Yarrowia

A strain that produces hexanoic acid that expresses TKS1P and OAC1P or HXS1 TKS1 OAC1 is grown overnight in 3 ml of YPD. This overnight culture is used to inoculate 25 ml of ScGP and grown overnight in a 250 ml flask at 225 rpm and 30° C. The biomass from the 25 mL culture is spun down and is washed with water. It is resuspended in 20 mL of ScP media with 800 μl of hexadecane or octadecane. Yeast cells, media and alkane are placed into a 250 mL culture flask. The culture flask is incubated at 30° C. while shaking (250 rpm) for 48 hours. The contents of the shake flask are spun down to produce a cell pellet and a supernatant, which is the culture media. The cell pellets and supernatants are analyzed for olivetolic acid using an LC MS MS method.

### Example 38—Production of CBGA from Olivetolic Acid in Yarrowia

YYL7 is grown overnight in 3 ml of ScD-leu. This overnight culture is used to inoculate 25 ml of ScGP-leu and grown overnight in a 250 ml flask at 225 rpm and 30° C. The biomass from the 25 mL culture is spun down and is washed with water, then resuspended in 20 mL of Sc-leu media with 800 μl oleic acid. Yeast cells, media and oleic are placed into a 250 mL culture flask. Olivetolic acid is added to a final concentration of 1 mM to the culture flask. The culture flask is incubated at 30° C. while shaking (250 rpm) for 48 hours. The contents of the shake flask are spun down to produce a cell pellet and a supernatant, which is the culture media. The cell pellets and supernatants are analyzed for CBGA using an LC-MS MS method.

### Example 39—Production of CBDA from Olivetolic Acid in Yarrowia

YYL17 is grown overnight in 3 ml of ScD-leu-ura. This overnight culture is used to inoculate 25 ml of ScGP-leu-ura and grown overnight in a 250 ml flask at 225 rpm and 30° C. The biomass from the 25 mL culture is spun down and is washed with water, then resuspended in 20 mL of ScP-ura-leu media with 800 μl oleic acid. Yeast cells, media and oleic are placed into a 250 mL culture flask. Olivetolic acid is added to a final concentration of 1 mM to the culture flask. The culture flask is incubated at 30° C. while shaking (250 rpm) for 48 hours. The contents of the shake flask are spun down to produce a cell pellet and a supernatant, which is the culture media. The cell pellets and supernatants are analyzed for CBDA using an LC-MS MS method.

### Example 40—Production of THCA from Olivetolic Acid in Yarrowia

A strain expressing PTS2 and THC1 is grown overnight in 3 ml of ScD-leu-ura (if genes are in a plasmid) or YPD (if genes are integrated). This overnight is used to inoculate 25 ml of ScGP-leu-ura if genes are in a plasmid) or ScGP (if genes are integrated) and grown overnight in a 250 ml flask at 225 rpm and 30° C. The biomass from the 25 mL culture is spun down and is washed with water, then resuspended in 20 mL of ScP-ura-leu media with 800 μl oleic acid (if genes are in a plasmid) or ScP (if genes are integrated) media with 800 μl oleic acid. Yeast cells, media and oleic are placed into a 250 mL culture flask. Olivetolic acid is added to a final concentration of 1 mM to the culture flask. The culture flask is incubated at 30° C. while shaking (250 rpm) for 48 hours. The contents of the shake flask are spun down to produce a cell pellet and a supernatant. The cell pellets and supernatants are analyzed for THCA using an LC-MS MS method.

### Example 41—Production of CBGA from Fatty Acids in Yarrowia

A strain that produces hexanoic acid that expresses TKS1P OAC1P PTS2 or HXS1 TKS1 OAC1 PTS2 is grown overnight in 3 ml of YPD. This overnight culture is used to inoculate 25 ml of ScGP and grown overnight in a 250 ml flask at 225 rpm and 30° C. The biomass from the 25 mL culture is spun down and is washed with water, then resuspended in 20 mL of ScP media with 800 μl oleic acid. Yeast cells, media and oleic acid are placed into a 250 mL culture flask. The culture flask is incubated at 30° C. while shaking (250 rpm) for 48 hours. The contents of the shake flask are spun down to produce a cell pellet and a supernatant. The cell pellets and supernatants are analyzed for CBGA using an LC-MS MS method.

### Example 42—Production of CBDA from Fatty Acids in Yarrowia

A strain that produces hexanoic acid that expresses TKS1P OAC1P PTS2 CBD1 or HXS1 TKS1 OAC1 PTS2 CBD1 is grown overnight in 3 ml of YPD. This overnight culture is used to inoculate 25 ml of ScGP and grown overnight in a 250 ml flask at 225 rpm and 30° C. The biomass from the 25 mL culture is spun down and is washed with water, then resuspended in 20 mL of ScP media with 800 μl oleic acid. Yeast cells, media and oleic acid are placed into a 250 mL culture flask. The culture flask is incubated at 30° C. while shaking (250 rpm) for 48 hours. The contents of the shake flask are spun down to produce a cell pellet and a supernatant. The cell pellets and supernatants are analyzed for CBDA using an LC-MS MS method.

### Example 43—Production of THCA from Fatty Acids in Yarrowia

A strain that produces hexanoic acid that expresses TKS1P OAC1P PTS2 THC1 or HXS1 TKS1 OAC1 PTS2 THC1 is grown overnight in 3 ml of YPD. This overnight culture is used to inoculate 25 ml of ScGP and grown overnight in a 250 ml flask at 225 rpm and 30° C. The biomass from the 25 mL culture is spun down and is washed with water, then resuspended in 20 mL of ScP media with 800 μl oleic acid. Yeast cells, media and oleic acid are placed into a 250 mL culture flask. The culture flask is incubated at 30° C. while shaking (250 rpm) for 48 hours. The contents of the shake flask are spun down to produce a cell pellet and a supernatant, which is the culture media. The cell pellets and supernatants are analyzed for THCA using an LC MS MS method.

### Example 44—Production of CBCA from Fatty Acids in Yarrowia

A strain that produces hexanoic acid that expresses TKS1P OAC1P PTS2 CBC1 or HXS1 TKS1 OAC1 PTS2 CBC1 is grown overnight in 3 ml of YPD. This overnight culture is used to inoculate 25 ml of ScGP and grown overnight in a 250 ml flask at 225 rpm and 30° C. The biomass from the 25 mL culture is spun down and is washed with water, then resuspended in 20 mL of ScP media with 800 μl oleic acid. Yeast cells, media and oleic acid are placed into a 250 mL culture flask. The culture flask is incubated at 30° C. while shaking (250 rpm) for 48 hours. The contents of the shake flask are spun down to produce a cell pellet and a supernatant, which is the culture media. The cell pellets and supernatants are analyzed for CBC1 using an LC MS MS method.

pLD1
(SEQ ID NO: 157)
TTAATTAATTCCGCTTAATGGAGTCCAAAAAGACCAACCTCTGCGCCTCGATCGACG
TGACCACAACCGCCGAGTTCCTTTCGCTCATCGACAAGCTCGGTCCCCACATCTGTC
TCGTGAAGACGCACATCGATATCATCTCAGACTTCAGCTACGAGGGCACGATTGAGC
CGTTGCTTGTGCTTGCAGAGCGCCACGGGTTCTTGATATTCGAGGACAGGAAGTTTG
CTGATATCGGAAACACCGTGATGTTGCAGTACACCTCGGGGGTATACCGGATCGCG
GCGTGGAGTGACATCACGAACGCGCACGGAGTGACTGGGAAGGGCGTCGTTGAAGG
GTTGAAACGCGGTGCGGAGGGGGTAGAAAAGGAAAGGGGCGTGTTGATGTTGGCG
GAGTTGTCGAGTAAAGGCTCGTTGGCGCATGGTGAATATACCCGTGAGACGATCGA
GATTGCGAAGAGTGATCGGGAGTTCGTGATTGGGTTCATCGCGCAGCGGGACATGG
GGGGTAGAGAAGAAGGGTTTGATTGGATCATCATGACGCCTGGTGTGGGGTTGGAT
GATAAAGGCGATGCGTTGGGCCAGCAGTATAGGACTGTTGATGAGGTGGTTCTGAC
TGGTACCGATGTGATTATTGTCGGGAGAGGGTTGTTTGGAAAAGGAAGAGACCCTG
AGGTGGAGGGAAAGAGATACAGGGATGCTGGATGGAAGGCATACTTGAAGAGAAC
TGGTCAGTTAGAATAAATATTGTAATAAATAGGTCTATATACATACACTAAGCTTCT
AGGACGTCATTGTAGTCTTCGAAGTTGTCTGCTAGTTTAGTTCTCATGATTTCGAAAA
CCAATAACGCAATGGATGTAGCAGGGATGGTGGTTAGTGCGTTCCTGACAAACCCA
GAGTACGCCGCCTCAAACCACGTCACATTCGCCCTTTGCTTCATCCGCATCACTTGCT
TGAAGGTATCCACGTACGAGTTGTAATACACCTTGAAGAACGGCTTCGTCTAGTTCG
GCATGGCAGATCATCATGCCTGCAGGAGCTCCAATTGTAATATTTCGGGAGAAATAT
CGTTGGGGTAAAACAACAGAGAGAGAGAGGGAGAGATGGTTCTGGTAGAATTATAA
TCTGGTTGTTGCAAATGCTACTGATCGACTCTGGCAATGTCTGTAGCTCGCTAGTTGT
ATGCAACTTAGGTGTTATGCATACACACGGTTATTCGGTTGAATTGTGGAGTAAAAA
TTGTCTGAGTTGTGTCTTAGCTACTGGCTGGCCCCCCGCGAAAGATAATCAAAATTA
CACTTGTGAATTTTTGCACACACACCGATTAACATTTCCCTTTTTTGTCCACCGATAC
ACGCTTGCCTCTTCTTATTTTCTCTGTGCTTCCCCCTCCTGTGACTTTTTCCACCATTG
ATATAAAATCAACTCCATTTCCCTAAAATCTCCCCAGATTCTAAAAACAACTTCTTCT
CTTCTGCTTTTCCTTATTTTTGTTATATTTATTTACCATCCCTTATTTTGAATAGTTATT
CCCCACTAACATTGTTCAAATCTTCACGACATAAGAAGAGCCCGGGTCTAGATGTGT
GCTCTTCCGAGTGACTCTTTTGATAAGAGTCGCAAATTTGATTTCATAAGTATATATT
CATTATGTAAAGTAGTAAATGGAAAATTCATTAAAAAAAAAGCAAATTTCCGTTGTA
TGCATACTCCGAACACAAAACTAGCCCCGGAAAAACCCTTAGTTGATAGTTGCGAAT
TTAGGTCGACCATATGCGACGGGTACAACGAGAATTGTATTGAATTGATCAAGAAC
ATGATCTTGGTGTTACAGAACATCAAGTTCTTGGACCAGACTGAGAATGCACAGATA
TACAAGGCGTCATGTGATAAAATGGATGAGATTTATCCACAATTGAAGAAAGAGTT
TATGGAAAGTGGTCAACCAGAAGCTAAACAGGAAGAAGCAAACGAAGAGGTGAAA
CAAGAAGAAGAAGGTAAATAAGTATTTTGTATTATATAACAAACAAAGTAAGGAAT
ACAGATTTATACAATAAATTGCCATACTAGTCACGTGAGATATCTCATCCATTCCCC
AACTCCCAAGAAAATAAAAAAGTGAAAAATAAAATCAAACCCAAAGATCAACCTCC
CCATCATCATCGTCATCAAACCCCCAGCTCAATTCGCAATGGTTAGCACAAAAACAT
ACACAGAAAGGGCATCAGCACACCCCTCCAAGGTTGCCCAACGTTTATTAATTAAA
GGCTAGGTGGAGGCTCAGTGATGATAAGTCTGCGATGGTGGATGCATGTGTCATGGT
CATAGCTGTTTCCTGTGTGAAATTGTTATCCGCTCAGAGGGCACAATCCTATTCCGC
GCTATCCGACAATCTCCAAGACATTAGGTGGAGTTCAGTTCGGCGTATGGCATATGT
CGCTGGAAAGAACATGTGAGCAAAAGGCCAGCAAAAGGCCAGGAACCGTAAAAAG
GCCGCGTTGCTGGCGTTTTTCCATAGGCTCCGCCCCCCTGACGAGCATCACAAAAAT
CGACGCTCAAGTCAGAGGTGGCGAAACCCGACAGGACTATAAAGATACCAGGCGTT
TCCCCCTGGAAGCTCCCTCGTGCGCTCTCCTGTTCCGACCCTGCCGCTTACCGGATAC
CTGTCCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCG
TTCAGCCCGACCGCTGCGCCTTATCCGGTAACTATCGTCTTGAGTCCAACCCGGTAA
GACACGACTTATCGCCACTGGCAGCAGCCACTGGTAACAGGATTAGCAGAGCGAGG
TATGTAGGCGGTGCTACAGAGTTCTTGAAGTGGTGGCCTAACTACGGCTACACTAGA
AGAACAGTATTTGGTATCTGCGCTCTGCTGAAGCCAGTTACCTTCGGAAAAAGAGTT
GGTAGCTCTTGATCCGGCAAACAAACCACCGCTGGTAGCGGTGGTTTTTTTGTTTGC
AAGCAGCAGATTACGCGCAGAAAAAAAGGATCTCAAGAAGATCCTTTGATCTTTTCT
ACGGGGTCTGACGCTCTATTCAACAAAGCCGCCGTCCCGTCAAGTCAGCGTAAATG
GGTAGGGGGCTTCAAATCGTCCGCTCTGCCAGTGTTACAACCAATTAACAAATTCTG
ATTAGAAAAACTCATCGAGCATCAAATGAAACTGCAATTTATTCATATCAGGATTAT
CAATACCATATTTTTGAAAAAGCCGTTTCTGTAATGAAGGAGAAAACTCACCGAGGC
AGTTCCATAGGATGGCAAGATCCTGGTATCGGTCTGCGATTCCGACTCGTCCAACAT
CAATACAACCTATTAATTTCCCCTCGTCAAAAATAAGGTTATCAAGTGAGAAATCAC
CATGAGTGACGACTGAATCCGGTGAGAATGGCAAAAGCTTATGCATTTCTTTCCAGA
CTTGTTCAACAGGCCAGCCATTACGCTCGTCATCAAAATCACTCGCATCAACCAAAC
CGTTATTCATTCGTGATTGCGCCTGAGCGAGACGAAATACGCGATCGCTGTTAAAAG
GACAATTACAAACAGGAATCGAATGCAACCGGCGCAGGAACACTGCCAGCGCATCA
ACAATATTTTCACCTGAATCAGGATATTCTTCTAATACCTGGAATGCTGTTTTCCCGG
GGATCGCAGTGGTGAGTAACCATGCATCATCAGGAGTACGGATAAAATGCTTGATG
GTCGGAAGAGGCATAAATTCCGTCAGCCAGTTTAGTCTGACCATCTCATCTGTAACA
TCATTGGCAACGCTACCTTTGCCATGTTTCAGAAACAACTCTGGCGCATCGGGCTTC
CCATACAATCGATAGATTGTCGCACCTGATTGCCCGACATTATCGCGAGCCCATTTA
TACCCATATAAATCAGCATCCATGTTGGAATTTAATCGCGGCCTCGAGCAAGACGTT
TCCCGTTGAATATGGCTCATAACACCCCTTGTATTACTGTTTATGTAAGCAGACAGTT
TTATTGTTCATGATGATATATTTTTATCTTGTGCAATGTAACATCAGAGATTTTGAGA
CACAACGTGGCTTTCCCCCGCCGCTCTAGAACTAGTGGATCCAAATAAAACGAAAG
GCTCAGTCGAAAGACTGGGCCTTTCGTTTTATCTGTTGTTTGTCGCATTATACGAGAC
GTCCAGGTTGGGATACCTGAAACAAAACCCATCGTACGGCCAAGGAAGTCTCCAAT
AACTGTGATCCACCACAAGCGCCAGGGTTTTCCCAGTCACGACGTTGTAAAACGAC
GGCCAGTCATGCATAATCCGCACGCATCTGGAATAAGGAAGTGCCATTCCGCCTGA
CCT
pLD10
(SEQ ID NO: 158)
TTAATTAATTCCGCTTAATGGAGTCCAAAAAGACCAACCTCTGCGCCTCGATCGACG
TGACCACAACCGCCGAGTTCCTTTCGCTCATCGACAAGCTCGGTCCCCACATCTGTC
TCGTGAAGACGCACATCGATATCATCTCAGACTTCAGCTACGAGGGCACGATTGAGC
CGTTGCTTGTGCTTGCAGAGCGCCACGGGTTCTTGATATTCGAGGACAGGAAGTTTG
CTGATATCGGAAACACCGTGATGTTGCAGTACACCTCGGGGGTATACCGGATCGCG
GCGTGGAGTGACATCACGAACGCGCACGGAGTGACTGGGAAGGGCGTCGTTGAAGG
GTTGAAACGCGGTGCGGAGGGGGTAGAAAAGGAAAGGGGCGTGTTGATGTTGGCG
GAGTTGTCGAGTAAAGGCTCGTTGGCGCATGGTGAATATACCCGTGAGACGATCGA
GATTGCGAAGAGTGATCGGGAGTTCGTGATTGGGTTCATCGCGCAGCGGGACATGG
GGGGTAGAGAAGAAGGGTTTGATTGGATCATCATGACGCCTGGTGTGGGGTTGGAT
GATAAAGGCGATGCGTTGGGCCAGCAGTATAGGACTGTTGATGAGGTGGTTCTGAC
TGGTACCGATGTGATTATTGTCGGGAGAGGGTTGTTTGGAAAAGGAAGAGACCCTG
AGGTGGAGGGAAAGAGATACAGGGATGCTGGATGGAAGGCATACTTGAAGAGAAC
TGGTCAGTTAGAATAAATATTGTAATAAATAGGTCTATATACATACACTAAGCTTCT
AGGACGTCATTGTAGTCTTCGAAGTTGTCTGCTAGTTTAGTTCTCATGATTTCGAAAA
CCAATAACGCAATGGATGTAGCAGGGATGGTGGTTAGTGCGTTCCTGACAAACCCA
GAGTACGCCGCCTCAAACCACGTCACATTCGCCCTTTGCTTCATCCGCATCACTTGCT
TGAAGGTATCCACGTACGAGTTGTAATACACCTTGAAGAACGGCTTCGTCTAGTTCG
GCATGGCAGATCATCATGCCTGCAGGAGCTCCAATTGTAATATTTCGGGAGAAATAT
CGTTGGGGTAAAACAACAGAGAGAGAGAGGGAGAGATGGTTCTGGTAGAATTATAA
TCTGGTTGTTGCAAATGCTACTGATCGACTCTGGCAATGTCTGTAGCTCGCTAGTTGT
ATGCAACTTAGGTGTTATGCATACACACGGTTATTCGGTTGAATTGTGGAGTAAAAA
TTGTCTGAGTTGTGTCTTAGCTACTGGCTGGCCCCCCGCGAAAGATAATCAAAATTA
CACTTGTGAATTTTTGCACACACACCGATTAACATTTCCCTTTTTTGTCCACCGATAC
ACGCTTGCCTCTTCTTATTTTCTCTGTGCTTCCCCCTCCTGTGACTTTTTCCACCATTG
ATATAAAATCAACTCCATTTCCCTAAAATCTCCCCAGATTCTAAAAACAACTTCTTCT
CTTCTGCTTTTCCTTATTTTTGTTATATTTATTTACCATCCCTTATTTTGAATAGTTATT
CCCCACTAACATTGTTCAAATCTTCACGACataATGAATCATTTAAGAGCAGAAGGAC
CCGCATCAGTGTTAGCGATAGGTACAGCTAACCCAGAGAATATCTTAATCCAAGATG
AATTTCCTGACTACTATTTCCGTGTTACTAAATCGGAACATATGACTCAACTTAAAG
AGAAGTTCCGGAAAATCTGCGATAAATCCATGATCCGAAAGAGAAACTGTTTCCTTA
ACGAAGAACATCTCAAGCAAAACCCGAGGTTGGTAGAGCACGAAATGCAGACCTTG
GATGCTAGGCAGGACATGTTGGTGGTCGAAGTGCCAAAACTCGGCAAGGACGCGTG
CGCTAAGGCAATCAAGGAGTGGGGTCAACCGAAGTCTAAAATCACGCATCTAATAT
TTACATCTGCACTGACAACCGACATGCCGGGTGCCGATTATCACTGCGCCAAGCTAC
TTGGATTGAGTCCACTGGTTAAGAGAGTTATGATGTATCAATTGGGGTGTTACGGAG
GGGGCACAGTCCTCAGAATTGCTAAGGATATTGCGGAAAATAACAAGGGCGCGAGG
GTCCTTGCTGTATGTTGTGATATTATGGCCTGTTTGTTTCGCGGGCCCTCGGATTCAG
ATTTGGAATTGCTTGTCGGACAGGCAATTTTTGGTGACGGGGCCGCAGCAGTCATAG
TGGGAGCCGAACCAGACGAAAGCGTGGGTGAAAGACCAATCTTTGAGTTGGTTCTG
ACCGGACAAACGATCTTACCTAACTCGGAAGGTACGATTGGAGGACATATTAGAGA
AGCCGGCCTAATTTTCGATCTTCACAAAGACGTTCCAATGTTAATCTCCAATAACAT
AGAAAAGTGCTTGATAGAAGCATTTACTCCCATTGGTATTAGTGACTGGAACAGCAT
TTTCTGGATCACCCACCCTGGAGGAAAAGCTATACTCGATAAGGTTGAAGAGAAAC
TCGACTTGAAAAAGGAGAAATTCGTTGACTCACGACATGTGTTATCAGAGCACGGG
AATATGAGTTCATCCACAGTCTTGTTCGTAATGGATGAATTGCGAAAACGCTCTCTT
GAGGAGGGAAAGAGCACAACCGGTGACGGGTTTGAGTGGGGCGTGCTATTCGGTTT
TGGCCCAGGTTTGACTGTCGAGCGGGTTGTTGTTCGTAGTGTACCAATTAAGTACTG
ATAAGAGTGACTCTTTTGATAAGAGTCGCAAATTTGATTTCATAAGTATATATTCATT
ATGTAAAGTAGTAAATGGAAAATTCATTAAAAAAAAAGCAAATTTCCGTTGTATGC
ATACTCCGAACACAAAACTAGCCCCGGAAAAACCCTTAGTTGATAGTTGCGAATTTA
GGTCGACCATATGCGACGGGTACAACGAGAATTGTATTGAATTGATCAAGAACATG
ATCTTGGTGTTACAGAACATCAAGTTCTTGGACCAGACTGAGAATGCACAGATATAC
AAGGCGTCATGTGATAAAATGGATGAGATTTATCCACAATTGAAGAAAGAGTTTAT
GGAAAGTGGTCAACCAGAAGCTAAACAGGAAGAAGCAAACGAAGAGGTGAAACAA
GAAGAAGAAGGTAAATAAGTATTTTGTATTATATAACAAACAAAGTAAGGAATACA
GATTTATACAATAAATTGCCATACTAGTCACGTGAGATATCTCATCCATTCCCCAAC
TCCCAAGAAAATAAAAAAGTGAAAAATAAAATCAAACCCAAAGATCAACCTCCCCA
TCATCATCGTCATCAAACCCCCAGCTCAATTCGCAATGGTTAGCACAAAAACATACA
CAGAAAGGGCATCAGCACACCCCTCCAAGGTTGCCCAACGTTTATTAATTAAAGGCT
AGGTGGAGGCTCAGTGATGATAAGTCTGCGATGGTGGATGCATGTGTCATGGTCATA
GCTGTTTCCTGTGTGAAATTGTTATCCGCTCAGAGGGCACAATCCTATTCCGCGCTAT
CCGACAATCTCCAAGACATTAGGTGGAGTTCAGTTCGGCGTATGGCATATGTCGCTG
GAAAGAACATGTGAGCAAAAGGCCAGCAAAAGGCCAGGAACCGTAAAAAGGCCGC
GTTGCTGGCGTTTTTCCATAGGCTCCGCCCCCCTGACGAGCATCACAAAAATCGACG
CTCAAGTCAGAGGTGGCGAAACCCGACAGGACTATAAAGATACCAGGCGTTTCCCC
CTGGAAGCTCCCTCGTGCGCTCTCCTGTTCCGACCCTGCCGCTTACCGGATACCTGTC
CGCCTTTCTCCCTTCGGGAAGCGTGGCGCTTTCTCATAGCTCACGCTGTAGGTATCTC
AGTTCGGTGTAGGTCGTTCGCTCCAAGCTGGGCTGTGTGCACGAACCCCCCGTTCAG
CCCGACCGCTGCGCCTTATCCGGTAACTATCGTCTTGAGTCCAACCCGGTAAGACAC
GACTTATCGCCACTGGCAGCAGCCACTGGTAACAGGATTAGCAGAGCGAGGTATGT
AGGCGGTGCTACAGAGTTCTTGAAGTGGTGGCCTAACTACGGCTACACTAGAAGAA
CAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTTTCTACGG
GGTCTGACGCTCTATTCAACAAAGCCGCCGTCCCGTCAAGTCAGCGTAAATGGGTAG
GGGGCTTCAAATCGTCCGCTCTGCCAGTGTTACAACCAATTAACAAATTCTGATTAG
AAAAACTCATCGAGCATCAAATGAAACTGCAATTTATTCATATCAGGATTATCAATA
CCATATTTTTGAAAAAGCCGTTTCTGTAATGAAGGAGAAAACTCACCGAGGCAGTTC
CATAGGATGGCAAGATCCTGGTATCGGTCTGCGATTCCGACTCGTCCAACATCAATA
CAACCTATTAATTTCCCCTCGTCAAAAATAAGGTTATCAAGTGAGAAATCACCATGA
GTGACGACTGAATCCGGTGAGAATGGCAAAAGCTTATGCATTTCTTTCCAGACTTGT
TCAACAGGCCAGCCATTACGCTCGTCATCAAAATCACTCGCATCAACCAAACCGTTA
TTCATTCGTGATTGCGCCTGAGCGAGACGAAATACGCGATCGCTGTTAAAAGGACA
ATTACAAACAGGAATCGAATGCAACCGGCGCAGGAACACTGCCAGCGCATCAACAA
TATTTTCACCTGAATCAGGATATTCTTCTAATACCTGGAATGCTGTTTTCCCGGGGAT
CGCAGTGGTGAGTAACCATGCATCATCAGGAGTACGGATAAAATGCTTGATGGTCG
GAAGAGGCATAAATTCCGTCAGCCAGTTTAGTCTGACCATCTCATCTGTAACATCAT
TGGCAACGCTACCTTTGCCATGTTTCAGAAACAACTCTGGCGCATCGGGCTTCCCAT
ACAATCGATAGATTGTCGCACCTGATTGCCCGACATTATCGCGAGCCCATTTATACC
CATATAAATCAGCATCCATGTTGGAATTTAATCGCGGCCTCGAGCAAGACGTTTCCC
GTTGAATATGGCTCATAACACCCCTTGTATTACTGTTTATGTAAGCAGACAGTTTTAT
TGTTCATGATGATATATTTTTATCTTGTGCAATGTAACATCAGAGATTTTGAGACACA
ACGTGGCTTTCCCCCGCCGCTCTAGAACTAGTGGATCCAAATAAAACGAAAGGCTC
AGTCGAAAGACTGGGCCTTTCGTTTTATCTGTTGTTTGTCGCATTATACGAGACGTCC
AGGTTGGGATACCTGAAACAAAACCCATCGTACGGCCAAGGAAGTCTCCAATAACT
GTGATCCACCACAAGCGCCAGGGTTTTCCCAGTCACGACGTTGTAAAACGACGGCC
AGTCATGCATAATCCGCACGCATCTGGAATAAGGAAGTGCCATTCCGCCTGACCT
pLD12
(SEQ ID NO: 159)
TTAATTAATTCCGCTTAATGGAGTCCAAAAAGACCAACCTCTGCGCCTCGATCGACG
TGACCACAACCGCCGAGTTCCTTTCGCTCATCGACAAGCTCGGTCCCCACATCTGTC
TCGTGAAGACGCACATCGATATCATCTCAGACTTCAGCTACGAGGGCACGATTGAGC
CGTTGCTTGTGCTTGCAGAGCGCCACGGGTTCTTGATATTCGAGGACAGGAAGTTTG
CTGATATCGGAAACACCGTGATGTTGCAGTACACCTCGGGGGTATACCGGATCGCG
GCGTGGAGTGACATCACGAACGCGCACGGAGTGACTGGGAAGGGCGTCGTTGAAGG
GTTGAAACGCGGTGCGGAGGGGGTAGAAAAGGAAAGGGGCGTGTTGATGTTGGCG
GAGTTGTCGAGTAAAGGCTCGTTGGCGCATGGTGAATATACCCGTGAGACGATCGA
GATTGCGAAGAGTGATCGGGAGTTCGTGATTGGGTTCATCGCGCAGCGGGACATGG
GGGGTAGAGAAGAAGGGTTTGATTGGATCATCATGACGCCTGGTGTGGGGTTGGAT
GATAAAGGCGATGCGTTGGGCCAGCAGTATAGGACTGTTGATGAGGTGGTTCTGAC
TGGTACCGATGTGATTATTGTCGGGAGAGGGTTGTTTGGAAAAGGAAGAGACCCTG
AGGTGGAGGGAAAGAGATACAGGGATGCTGGATGGAAGGCATACTTGAAGAGAAC
TGGTCAGTTAGAATAAATATTGTAATAAATAGGTCTATATACATACACTAAGCTTCT
AGGACGTCATTGTAGTCTTCGAAGTTGTCTGCTAGTTTAGTTCTCATGATTTCGAAAA
CCAATAACGCAATGGATGTAGCAGGGATGGTGGTTAGTGCGTTCCTGACAAACCCA
GAGTACGCCGCCTCAAACCACGTCACATTCGCCCTTTGCTTCATCCGCATCACTTGCT
TGAAGGTATCCACGTACGAGTTGTAATACACCTTGAAGAACGGCTTCGTCTAGTTCG
GCATGGCAGATCATCATGCCTGCAGGAGCTCCAATTGTAATATTTCGGGAGAAATAT
CGTTGGGGTAAAACAACAGAGAGAGAGAGGGAGAGATGGTTCTGGTAGAATTATAA
TCTGGTTGTTGCAAATGCTACTGATCGACTCTGGCAATGTCTGTAGCTCGCTAGTTGT
ATGCAACTTAGGTGTTATGCATACACACGGTTATTCGGTTGAATTGTGGAGTAAAAA
TTGTCTGAGTTGTGTCTTAGCTACTGGCTGGCCCCCCGCGAAAGATAATCAAAATTA
CACTTGTGAATTTTTGCACACACACCGATTAACATTTCCCTTTTTTGTCCACCGATAC
ACGCTTGCCTCTTCTTATTTTCTCTGTGCTTCCCCCTCCTGTGACTTTTTCCACCATTG
ATATAAAATCAACTCCATTTCCCTAAAATCTCCCCAGATTCTAAAAACAACTTCTTCT
CTTCTGCTTTTCCTTATTTTTGTTATATTTATTTACCATCCCTTATTTTGAATAGTTATT
CCCCACTAACATTGTTCAAATCTTCACGACataATGGCAGTCAAACACCTAATAGTTCT
CAAATTTAAAGACGAGATTACTGAAGCTCAGAAGGAAGAGTTCTTTAAGACATATG
TTAACTTAGTCAACATCATCCCCGCGATGAAGGACGTCTACTGGGGCAAGGATGTGA
CGCAAAAAAATAAGGAAGAAGGATACACACATATCGTTGAGGTGACCTTTGAGAGT
GTGGAAACTATTCAAGATTATATTATTCACCCAGCCCATGTAGGGTTCGGTGACGTT
TATCGATCATTCTGGGAAAAGTTGCTTATATTTGATTACACCCCAAGAAAATTGAAG
CCTAAGTGATAAGAGTGACTCTTTTGATAAGAGTCGCAAATTTGATTTCATAAGTAT
ATATTCATTATGTAAAGTAGTAAATGGAAAATTCATTAAAAAAAAAGCAAATTTCCG
TTGTATGCATACTCCGAACACAAAACTAGCCCCGGAAAAACCCTTAGTTGATAGTTG
CGAATTTAGGTCGACCATATGCGACGGGTACAACGAGAATTGTATTGAATTGATCAA
GAACATGATCTTGGTGTTACAGAACATCAAGTTCTTGGACCAGACTGAGAATGCACA
GATATACAAGGCGTCATGTGATAAAATGGATGAGATTTATCCACAATTGAAGAAAG
AGTTTATGGAAAGTGGTCAACCAGAAGCTAAACAGGAAGAAGCAAACGAAGAGGT
GAAACAAGAAGAAGAAGGTAAATAAGTATTTTGTATTATATAACAAACAAAGTAAG
GAATACAGATTTATACAATAAATTGCCATACTAGTCACGTGAGATATCTCATCCATT
CCCCAACTCCCAAGAAAATAAAAAAGTGAAAAATAAAATCAAACCCAAAGATCAA
CCTCCCCATCATCATCGTCATCAAACCCCCAGCTCAATTCGCAATGGTTAGCACAAA
AACATACACAGAAAGGGCATCAGCACACCCCTCCAAGGTTGCCCAACGTTTATTAA
TTAAAGGCTAGGTGGAGGCTCAGTGATGATAAGTCTGCGATGGTGGATGCATGTGTC
ATGGTCATAGCTGTTTCCTGTGTGAAATTGTTATCCGCTCAGAGGGCACAATCCTATT
CCGCGCTATCCGACAATCTCCAAGACATTAGGTGGAGTTCAGTTCGGCGTATGGCAT
ATGTCGCTGGAAAGAACATGTGAGCAAAAGGCCAGCAAAAGGCCAGGAACCGTAA
AAAGGCCGCGTTGCTGGCGTTTTTCCATAGGCTCCGCCCCCCTGACGAGCATCACAA
AAATCGACGCTCAAGTCAGAGGTGGCGAAACCCGACAGGACTATAAAGATACCAGG
CGTTTCCCCCTGGAAGCTCCCTCGTGCGCTCTCCTGTTCCGACCCTGCCGCTTACCGG
ATACCTGTCCGCCTTTCTCCCTTCGGGAAGCGTGGCGCTTTCTCATAGCTCACGCTGT
AGGTATCTCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTCCAACCCG
GTAAGACACGACTTATCGCCACTGGCAGCAGCCACTGGTAACAGGATTAGCAGAGC
GAGGTATGTAGGCGGTGCTACAGAGTTCTTGAAGTGGTGGCCTAACTACGGCTACAC
TAGAAGAACAGTATTTGGTATCTGCGCTCTGCTGAAGCCAGTTACCTTCGGAAAAAG
AGTTGGTAGCTCTTGATCCGGCAAACAAACCACCGCTGGTAGCGGTGGTTTTTTTGT
TTGCAAGCAGCAGATTACGCGCAGAAAAAAAGGATCTCAAGAAGATCCTTTGATCT
TTTCTACGGGGTCTGACGCTCTATTCAACAAAGCCGCCGTCCCGTCAAGTCAGCGTA
AATGGGTAGGGGGCTTCAAATCGTCCGCTCTGCCAGTGTTACAACCAATTAACAAAT
TCTGATTAGAAAAACTCATCGAGCATCAAATGAAACTGCAATTTATTCATATCAGGA
TTATCAATACCATATTTTTGAAAAAGCCGTTTCTGTAATGAAGGAGAAAACTCACCG
AGGCAGTTCCATAGGATGGCAAGATCCTGGTATCGGTCTGCGATTCCGACTCGTCCA
ACATCAATACAACCTATTAATTTCCCCTCGTCAAAAATAAGGTTATCAAGTGAGAAA
TCACCATGAGTGACGACTGAATCCGGTGAGAATGGCAAAAGCTTATGCATTTCTTTC
CAGACTTGTTCAACAGGCCAGCCATTACGCTCGTCATCAAAATCACTCGCATCAACC
AAACCGTTATTCATTCGTGATTGCGCCTGAGCGAGACGAAATACGCGATCGCTGTTA
AAAGGACAATTACAAACAGGAATCGAATGCAACCGGCGCAGGAACACTGCCAGCG
CATCAACAATATTTTCACCTGAATCAGGATATTCTTCTAATACCTGGAATGCTGTTTT
CCCGGGGATCGCAGTGGTGAGTAACCATGCATCATCAGGAGTACGGATAAAATGCT
TGATGGTCGGAAGAGGCATAAATTCCGTCAGCCAGTTTAGTCTGACCATCTCATCTG
TAACATCATTGGCAACGCTACCTTTGCCATGTTTCAGAAACAACTCTGGCGCATCGG
GCTTCCCATACAATCGATAGATTGTCGCACCTGATTGCCCGACATTATCGCGAGCCC
ATTTATACCCATATAAATCAGCATCCATGTTGGAATTTAATCGCGGCCTCGAGCAAG
ACGTTTCCCGTTGAATATGGCTCATAACACCCCTTGTATTACTGTTTATGTAAGCAGA
CAGTTTTATTGTTCATGATGATATATTTTTATCTTGTGCAATGTAACATCAGAGATTT
TGAGACACAACGTGGCTTTCCCCCGCCGCTCTAGAACTAGTGGATCCAAATAAAAC
GAAAGGCTCAGTCGAAAGACTGGGCCTTTCGTTTTATCTGTTGTTTGTCGCATTATAC
GAGACGTCCAGGTTGGGATACCTGAAACAAAACCCATCGTACGGCCAAGGAAGTCT
CCAATAACTGTGATCCACCACAAGCGCCAGGGTTTTCCCAGTCACGACGTTGTAAAA
CGACGGCCAGTCATGCATAATCCGCACGCATCTGGAATAAGGAAGTGCCATTCCGC
CTGACCT
pLD14
(SEQ ID NO: 160)
TTAATTAATTCCGCTTAATGGAGTCCAAAAAGACCAACCTCTGCGCCTCGATCGACG
TGACCACAACCGCCGAGTTCCTTTCGCTCATCGACAAGCTCGGTCCCCACATCTGTC
TCGTGAAGACGCACATCGATATCATCTCAGACTTCAGCTACGAGGGCACGATTGAGC
CGTTGCTTGTGCTTGCAGAGCGCCACGGGTTCTTGATATTCGAGGACAGGAAGTTTG
CTGATATCGGAAACACCGTGATGTTGCAGTACACCTCGGGGGTATACCGGATCGCG
GCGTGGAGTGACATCACGAACGCGCACGGAGTGACTGGGAAGGGCGTCGTTGAAGG
GTTGAAACGCGGTGCGGAGGGGGTAGAAAAGGAAAGGGGCGTGTTGATGTTGGCG
GAGTTGTCGAGTAAAGGCTCGTTGGCGCATGGTGAATATACCCGTGAGACGATCGA
GATTGCGAAGAGTGATCGGGAGTTCGTGATTGGGTTCATCGCGCAGCGGGACATGG
GGGGTAGAGAAGAAGGGTTTGATTGGATCATCATGACGCCTGGTGTGGGGTTGGAT
GATAAAGGCGATGCGTTGGGCCAGCAGTATAGGACTGTTGATGAGGTGGTTCTGAC
TGGTACCGATGTGATTATTGTCGGGAGAGGGTTGTTTGGAAAAGGAAGAGACCCTG
AGGTGGAGGGAAAGAGATACAGGGATGCTGGATGGAAGGCATACTTGAAGAGAAC
TGGTCAGTTAGAATAAATATTGTAATAAATAGGTCTATATACATACACTAAGCTTCT
AGGACGTCATTGTAGTCTTCGAAGTTGTCTGCTAGTTTAGTTCTCATGATTTCGAAAA
CCAATAACGCAATGGATGTAGCAGGGATGGTGGTTAGTGCGTTCCTGACAAACCCA
GAGTACGCCGCCTCAAACCACGTCACATTCGCCCTTTGCTTCATCCGCATCACTTGCT
TGAAGGTATCCACGTACGAGTTGTAATACACCTTGAAGAACGGCTTCGTCTAGTTCG
GCATGGCAGATCATCATGCCTGCAGGAGCTCCAATTGTAATATTTCGGGAGAAATAT
CGTTGGGGTAAAACAACAGAGAGAGAGAGGGAGAGATGGTTCTGGTAGAATTATAA
TCTGGTTGTTGCAAATGCTACTGATCGACTCTGGCAATGTCTGTAGCTCGCTAGTTGT
ATGCAACTTAGGTGTTATGCATACACACGGTTATTCGGTTGAATTGTGGAGTAAAAA
TTGTCTGAGTTGTGTCTTAGCTACTGGCTGGCCCCCCGCGAAAGATAATCAAAATTA
CACTTGTGAATTTTTGCACACACACCGATTAACATTTCCCTTTTTTGTCCACCGATAC
ACGCTTGCCTCTTCTTATTTTCTCTGTGCTTCCCCCTCCTGTGACTTTTTCCACCATTG
ATATAAAATCAACTCCATTTCCCTAAAATCTCCCCAGATTCTAAAAACAACTTCTTCT
CTTCTGCTTTTCCTTATTTTTGTTATATTTATTTACCATCCCTTATTTTGAATAGTTATT
CCCCACTAACATTGTTCAAATCTTCACGACataATGGCAGTCAAACACCTAATAGTTCT
CAAATTTAAAGACGAGATTACTGAAGCTCAGAAGGAAGAGTTCTTTAAGACATATG
TTAACTTAGTCAACATCATCCCCGCGATGAAGGACGTCTACTGGGGCAAGGATGTGA
CGCAAAAAAATAAGGAAGAAGGATACACACATATCGTTGAGGTGACCTTTGAGAGT
GTGGAAACTATTCAAGATTATATTATTCACCCAGCCCATGTAGGGTTCGGTGACGTT
TATCGATCATTCTGGGAAAAGTTGCTTATATTTGATTACACCCCAAGAAAATTGAAG
CCTAAGGGAAGACGAGCTAAGTTGTGATAAGAGTGACTCTTTTGATAAGAGTCGCA
AATTTGATTTCATAAGTATATATTCATTATGTAAAGTAGTAAATGGAAAATTCATTA
AAAAAAAAGCAAATTTCCGTTGTATGCATACTCCGAACACAAAACTAGCCCCGGAA
AAACCCTTAGTTGATAGTTGCGAATTTAGGTCGACCATATGCGACGGGTACAACGAG
AATTGTATTGAATTGATCAAGAACATGATCTTGGTGTTACAGAACATCAAGTTCTTG
GACCAGACTGAGAATGCACAGATATACAAGGCGTCATGTGATAAAATGGATGAGAT
TTATCCACAATTGAAGAAAGAGTTTATGGAAAGTGGTCAACCAGAAGCTAAACAGG
AAGAAGCAAACGAAGAGGTGAAACAAGAAGAAGAAGGTAAATAAGTATTTTGTAT
TATATAACAAACAAAGTAAGGAATACAGATTTATACAATAAATTGCCATACTAGTC
ACGTGAGATATCTCATCCATTCCCCAACTCCCAAGAAAATAAAAAAGTGAAAAATA
AAATCAAACCCAAAGATCAACCTCCCCATCATCATCGTCATCAAACCCCCAGCTCAA
TTCGCAATGGTTAGCACAAAAACATACACAGAAAGGGCATCAGCACACCCCTCCAA
GGTTGCCCAACGTTTATTAATTAAAGGCTAGGTGGAGGCTCAGTGATGATAAGTCTG
CGATGGTGGATGCATGTGTCATGGTCATAGCTGTTTCCTGTGTGAAATTGTTATCCGC
TCAGAGGGCACAATCCTATTCCGCGCTATCCGACAATCTCCAAGACATTAGGTGGAG
TTCAGTTCGGCGTATGGCATATGTCGCTGGAAAGAACATGTGAGCAAAAGGCCAGC
AAAAGGCCAGGAACCGTAAAAAGGCCGCGTTGCTGGCGTTTTTCCATAGGCTCCGC
CCCCCTGACGAGCATCACAAAAATCGACGCTCAAGTCAGAGGTGGCGAAACCCGAC
AGGACTATAAAGATACCAGGCGTTTCCCCCTGGAAGCTCCCTCGTGCGCTCTCCTGT
TCCGACCCTGCCGCTTACCGGATACCTGTCCGCCTTTCTCCCTTCGGGAAGCGTGGC
GCTTTCTCATAGCTCACGCTGTAGGTATCTCAGTTCGGTGTAGGTCGTTCGCTCCAAG
CTGGGCTGTGTGCACGAACCCCCCGTTCAGCCCGACCGCTGCGCCTTATCCGGTAAC
TATCGTCTTGAGTCCAACCCGGTAAGACACGACTTATCGCCACTGGCAGCAGCCACT
GGTAACAGGATTAGCAGAGCGAGGTATGTAGGCGGTGCTACAGAGTTCTTGAAGTG
GTGGCCTAACTACGGCTACACTAGAAGAACAGTATTTGGTATCTGCGCTCTGCTGAA
GCCAGTTACCTTCGGAAAAAGAGTTGGTAGCTCTTGATCCGGCAAACAAACCACCG
CTGGTAGCGGTGGTTTTTTTGTTTGCAAGCAGCAGATTACGCGCAGAAAAAAAGGAT
CTCAAGAAGATCCTTTGATCTTTTCTACGGGGTCTGACGCTCTATTCAACAAAGCCG
CCGTCCCGTCAAGTCAGCGTAAATGGGTAGGGGGCTTCAAATCGTCCGCTCTGCCAG
TGTTACAACCAATTAACAAATTCTGATTAGAAAAACTCATCGAGCATCAAATGAAAC
TGCAATTTATTCATATCAGGATTATCAATACCATATTTTTGAAAAAGCCGTTTCTGTA
ATGAAGGAGAAAACTCACCGAGGCAGTTCCATAGGATGGCAAGATCCTGGTATCGG
TCTGCGATTCCGACTCGTCCAACATCAATACAACCTATTAATTTCCCCTCGTCAAAA
ATAAGGTTATCAAGTGAGAAATCACCATGAGTGACGACTGAATCCGGTGAGAATGG
CAAAAGCTTATGCATTTCTTTCCAGACTTGTTCAACAGGCCAGCCATTACGCTCGTC
ATCAAAATCACTCGCATCAACCAAACCGTTATTCATTCGTGATTGCGCCTGAGCGAG
ACGAAATACGCGATCGCTGTTAAAAGGACAATTACAAACAGGAATCGAATGCAACC
GGCGCAGGAACACTGCCAGCGCATCAACAATATTTTCACCTGAATCAGGATATTCTT
CTAATACCTGGAATGCTGTTTTCCCGGGGATCGCAGTGGTGAGTAACCATGCATCAT
CAGGAGTACGGATAAAATGCTTGATGGTCGGAAGAGGCATAAATTCCGTCAGCCAG
TTTAGTCTGACCATCTCATCTGTAACATCATTGGCAACGCTACCTTTGCCATGTTTCA
GAAACAACTCTGGCGCATCGGGCTTCCCATACAATCGATAGATTGTCGCACCTGATT
GCCCGACATTATCGCGAGCCCATTTATACCCATATAAATCAGCATCCATGTTGGAAT
TTAATCGCGGCCTCGAGCAAGACGTTTCCCGTTGAATATGGCTCATAACACCCCTTG
TATTACTGTTTATGTAAGCAGACAGTTTTATTGTTCATGATGATATATTTTTATCTTGT
GCAATGTAACATCAGAGATTTTGAGACACAACGTGGCTTTCCCCCGCCGCTCTAGAA
CTAGTGGATCCAAATAAAACGAAAGGCTCAGTCGAAAGACTGGGCCTTTCGTTTTAT
CTGTTGTTTGTCGCATTATACGAGACGTCCAGGTTGGGATACCTGAAACAAAACCCA
TCGTACGGCCAAGGAAGTCTCCAATAACTGTGATCCACCACAAGCGCCAGGGTTTTC
CCAGTCACGACGTTGTAAAACGACGGCCAGTCATGCATAATCCGCACGCATCTGGA
ATAAGGAAGTGCCATTCCGCCTGACCT
pLD16
(SEQ ID NO: 161)
TTAATTAATTCCGCTTAATGGAGTCCAAAAAGACCAACCTCTGCGCCTCGATCGACG
TGACCACAACCGCCGAGTTCCTTTCGCTCATCGACAAGCTCGGTCCCCACATCTGTC
TCGTGAAGACGCACATCGATATCATCTCAGACTTCAGCTACGAGGGCACGATTGAGC
CGTTGCTTGTGCTTGCAGAGCGCCACGGGTTCTTGATATTCGAGGACAGGAAGTTTG
CTGATATCGGAAACACCGTGATGTTGCAGTACACCTCGGGGGTATACCGGATCGCG
GCGTGGAGTGACATCACGAACGCGCACGGAGTGACTGGGAAGGGCGTCGTTGAAGG
GTTGAAACGCGGTGCGGAGGGGGTAGAAAAGGAAAGGGGCGTGTTGATGTTGGCG
GAGTTGTCGAGTAAAGGCTCGTTGGCGCATGGTGAATATACCCGTGAGACGATCGA
GATTGCGAAGAGTGATCGGGAGTTCGTGATTGGGTTCATCGCGCAGCGGGACATGG
GGGGTAGAGAAGAAGGGTTTGATTGGATCATCATGACGCCTGGTGTGGGGTTGGAT
GATAAAGGCGATGCGTTGGGCCAGCAGTATAGGACTGTTGATGAGGTGGTTCTGAC
TGGTACCGATGTGATTATTGTCGGGAGAGGGTTGTTTGGAAAAGGAAGAGACCCTG
AGGTGGAGGGAAAGAGATACAGGGATGCTGGATGGAAGGCATACTTGAAGAGAAC
TGGTCAGTTAGAATAAATATTGTAATAAATAGGTCTATATACATACACTAAGCTTCT
AGGACGTCATTGTAGTCTTCGAAGTTGTCTGCTAGTTTAGTTCTCATGATTTCGAAAA
CCAATAACGCAATGGATGTAGCAGGGATGGTGGTTAGTGCGTTCCTGACAAACCCA
GAGTACGCCGCCTCAAACCACGTCACATTCGCCCTTTGCTTCATCCGCATCACTTGCT
TGAAGGTATCCACGTACGAGTTGTAATACACCTTGAAGAACGGCTTCGTCTAGTTCG
GCATGGCAGATCATCATGCCTGCAGGAGCTCCAATTGTAATATTTCGGGAGAAATAT
CGTTGGGGTAAAACAACAGAGAGAGAGAGGGAGAGATGGTTCTGGTAGAATTATAA
TCTGGTTGTTGCAAATGCTACTGATCGACTCTGGCAATGTCTGTAGCTCGCTAGTTGT
ATGCAACTTAGGTGTTATGCATACACACGGTTATTCGGTTGAATTGTGGAGTAAAAA
TTGTCTGAGTTGTGTCTTAGCTACTGGCTGGCCCCCCGCGAAAGATAATCAAAATTA
CACTTGTGAATTTTTGCACACACACCGATTAACATTTCCCTTTTTTGTCCACCGATAC
ACGCTTGCCTCTTCTTATTTTCTCTGTGCTTCCCCCTCCTGTGACTTTTTCCACCATTG
ATATAAAATCAACTCCATTTCCCTAAAATCTCCCCAGATTCTAAAAACAACTTCTTCT
CTTCTGCTTTTCCTTATTTTTGTTATATTTATTTACCATCCCTTATTTTGAATAGTTATT
CCCCACTAACATTGTTCAAATCTTCACGACataATGGGAAAAAATTATAAATCTTTGG
ACTCAGTTGTGGCTAGTGACTTCATTGCACTTGGGATCACATCAGAAGTTGCTGAGA
CATTGCACGGACGCTTGGCAGAGATAGTTTGCAACTACGGCGCCGCAACACCTCAG
ACCTGGATTAACATCGCAAACCATATTCTAAGTCCAGATCTTCCATTTAGTCTCCATC
AGATGTTGTTCTACGGTTGTTATAAGGACTTTGGTCCAGCACCCCCAGCTTGGATAC
CAGACCCCGAAAAAGTAAAGTCCACGAACTTAGGTGCCTTGTTAGAAAAGCGGGGA
AAGGAGTTTCTAGGCGTTAAGTATAAGGACCCAATAAGTCTGTTTTCTCACTTCCAG
GAGTTTAGCGTTCGAAATCCGGAAGTCTACTGGCGGACGGTACTTATGGATGAAATG
AAGATACTGTTCAGCAAAGATCCCGAATGTATCCTCAGACGCGACGACATTAACAA
CCCAGGGGGCTCTGAGTGGCTACCAGGTGGATATCTCAACCTGGCCAAGAACTGTTT
GAATGTAAATAGTAACAAAAAACTTAACGACACTATGATAGTGTGGAGAGATGAAG
GAAATGACGATCTCCCATTGAATAAATTGACTCTTGATCAATTACGAAAACGAGTCT
GGTTGGTTGGATACGCCCTAGAAGAGATGGGCCTTGAGAAGGGATGTGCGATTGCA
ATTGACATGCCCATGCACGTAGATGCGGTTGTGATCTATTTAGCTATCGTCTTGGCA
GGCTACGTCGTTGTCTCCATTGCAGATTCATTCTCAGCACCGGAAATTTCCACAAGA
TTGCGTCTATCAAAGGCTAAGGCTATTTTTACACAAGATCATATCATCCGAGGGAAA
AAGCGTATACCTTTGTACCTGCGTGTCGTCGAGGCCAAGTCTCCGATGGCAATAGTT
ATCCCGTGTTCGGGTTCAAATATTGGTGCGGAATTGCGGGATGGTGATATTCTGTGG
GATTACTTCTTAGAACGCGCAAAGGAATTTAAGAACTGCGAATTTACAGCCCGTGAA
CAGCCAGTGGACGCGTACACAAATATTTTGTTCTCATCGGGAACCACCGGAGAGCC
AAAGGCGATACCATGGACTCAAGCTACGCCTCTCAAGGCGGCTGCTGATGGTTGGTC
ACACTTGGACATTAGAAAGGGTGACGTAATTGTATGGCCTACCAATTTGGGGTGGAT
GATGGGGCCTTGGTTGGTCTATGCTTCACTCCTTAACGGGGCAAGCATCGCATTGTA
TAACGGATCTCCACTAGTGTCCGGCTTTGCCAAATTCGTTCAAGATGCGAAAGTTAC
TATGCTAGGAGTTGTCCCCTCCATCGTACGAAGCTGGAAAAGCACTAATTGCGTTAG
TGGGTACGATTGGTCTACAATCAGATGCTTCTCCTCATCGGGTGAGGCATCGAATGT
CGATGAATACTTATGGCTAATGGGAAGGGCTAACTACAAACCGGTCATCGAAATGT
GCGGTGGCACAGAGATCGGGGGTGCCTTCAGCGCCGGTTCGTTTTTACAAGCCCAAT
CTTTGAGTAGCTTCTCATCCCAATGTATGGGATGCACCTTGTACATTCTCGACAAGA
ATGGCTACCCGATGCCAAAGAACAAGCCGGGTATAGGTGAATTGGCCTTGGGACCC
GTGATGTTCGGTGCTTCCAAGACTTTACTTAACGGAAACCATCATGACGTTTATTTCA
AAGGCATGCCCACCTTGAACGGAGAAGTCTTGAGGAGACACGGAGATATCTTCGAA
CTCACTTCGAACGGCTATTATCACGCTCATGGTAGAGCAGATGACACGATGAATATC
GGGGGGATTAAAATTTCCTCAATCGAGATTGAAAGGGTGTGTAATGAAGTTGACGA
TAGAGTGTTTGAGACTACGGCCATTGGAGTGCCTCCATTGGGCGGAGGTCCAGAGC
AGCTCGTTATCTTTTTTGTTCTTAAGGACAGCAATGATACGACCATCGACCTAAACC
AATTGCGACTTAGTTTTAATCTTGGGTTACAAAAGAAATTGAACCCACTTTTTAAGG
TGACGAGGGTTGTGCCACTTTCGCTGTTGCCTAGGACAGCCACCAACAAAATAATGA
GAAGAGTGCTTAGACAGCAATTTAGTCATTTCGAGTGATAAGAGTGACTCTTTTGAT
AAGAGTCGCAAATTTGATTTCATAAGTATATATTCATTATGTAAAGTAGTAAATGGA
AAATTCATTAAAAAAAAAGCAAATTTCCGTTGTATGCATACTCCGAACACAAAACTA
GCCCCGGAAAAACCCTTAGTTGATAGTTGCGAATTTAGGTCGACCATATGCGACGGG
TACAACGAGAATTGTATTGAATTGATCAAGAACATGATCTTGGTGTTACAGAACATC
AAGTTCTTGGACCAGACTGAGAATGCACAGATATACAAGGCGTCATGTGATAAAAT
GGATGAGATTTATCCACAATTGAAGAAAGAGTTTATGGAAAGTGGTCAACCAGAAG
CTAAACAGGAAGAAGCAAACGAAGAGGTGAAACAAGAAGAAGAAGGTAAATAAGT
ATTTTGTATTATATAACAAACAAAGTAAGGAATACAGATTTATACAATAAATTGCCA
TACTAGTCACGTGAGATATCTCATCCATTCCCCAACTCCCAAGAAAATAAAAAAGTG
AAAAATAAAATCAAACCCAAAGATCAACCTCCCCATCATCATCGTCATCAAACCCC
CAGCTCAATTCGCAATGGTTAGCACAAAAACATACACAGAAAGGGCATCAGCACAC
CCCTCCAAGGTTGCCCAACGTTTATTAATTAAAGGCTAGGTGGAGGCTCAGTGATGA
TAAGTCTGCGATGGTGGATGCATGTGTCATGGTCATAGCTGTTTCCTGTGTGAAATT
GTTATCCGCTCAGAGGGCACAATCCTATTCCGCGCTATCCGACAATCTCCAAGACAT
TAGGTGGAGTTCAGTTCGGCGTATGGCATATGTCGCTGGAAAGAACATGTGAGCAA
AAGGCCAGCAAAAGGCCAGGAACCGTAAAAAGGCCGCGTTGCTGGCGTTTTTCCAT
AGGCTCCGCCCCCCTGACGAGCATCACAAAAATCGACGCTCAAGTCAGAGGTGGCG
AAACCCGACAGGACTATAAAGATACCAGGCGTTTCCCCCTGGAAGCTCCCTCGTGC
GCTCTCCTGTTCCGACCCTGCCGCTTACCGGATACCTGTCCGCCTTTCTCCCTTCGGG
AAGCGTGGCGCTTTCTCATAGCTCACGCTGTAGGTATCTCAGTTCGGTGTAGGTCGT
TCGCTCCAAGCTGGGCTGTGTGCACGAACCCCCCGTTCAGCCCGACCGCTGCGCCTT
ATCCGGTAACTATCGTCTTGAGTCCAACCCGGTAAGACACGACTTATCGCCACTGGC
AGCAGCCACTGGTAACAGGATTAGCAGAGCGAGGTATGTAGGCGGTGCTACAGAGT
TCTTGAAGTGGTGGCCTAACTACGGCTACACTAGAAGAACAGTATTTGGTATCTGCG
CTCTGCTGAAGCCAGTTACCTTCGGAAAAAGAGTTGGTAGCTCTTGATCCGGCAAAC
AAACCACCGCTGGTAGCGGTGGTTTTTTTGTTTGCAAGCAGCAGATTACGCGCAGAA
AAAAAGGATCTCAAGAAGATCCTTTGATCTTTTCTACGGGGTCTGACGCTCTATTCA
ACAAAGCCGCCGTCCCGTCAAGTCAGCGTAAATGGGTAGGGGGCTTCAAATCGTCC
GCTCTGCCAGTGTTACAACCAATTAACAAATTCTGATTAGAAAAACTCATCGAGCAT
CAAATGAAACTGCAATTTATTCATATCAGGATTATCAATACCATATTTTTGAAAAAG
CCGTTTCTGTAATGAAGGAGAAAACTCACCGAGGCAGTTCCATAGGATGGCAAGAT
CCTGGTATCGGTCTGCGATTCCGACTCGTCCAACATCAATACAACCTATTAATTTCCC
CTCGTCAAAAATAAGGTTATCAAGTGAGAAATCACCATGAGTGACGACTGAATCCG
GTGAGAATGGCAAAAGCTTATGCATTTCTTTCCAGACTTGTTCAACAGGCCAGCCAT
TACGCTCGTCATCAAAATCACTCGCATCAACCAAACCGTTATTCATTCGTGATTGCG
CCTGAGCGAGACGAAATACGCGATCGCTGTTAAAAGGACAATTACAAACAGGAATC
GAATGCAACCGGCGCAGGAACACTGCCAGCGCATCAACAATATTTTCACCTGAATC
AGGATATTCTTCTAATACCTGGAATGCTGTTTTCCCGGGGATCGCAGTGGTGAGTAA
CCATGCATCATCAGGAGTACGGATAAAATGCTTGATGGTCGGAAGAGGCATAAATT
CCGTCAGCCAGTTTAGTCTGACCATCTCATCTGTAACATCATTGGCAACGCTACCTTT
GCCATGTTTCAGAAACAACTCTGGCGCATCGGGCTTCCCATACAATCGATAGATTGT
CGCACCTGATTGCCCGACATTATCGCGAGCCCATTTATACCCATATAAATCAGCATC
CATGTTGGAATTTAATCGCGGCCTCGAGCAAGACGTTTCCCGTTGAATATGGCTCAT
AACACCCCTTGTATTACTGTTTATGTAAGCAGACAGTTTTATTGTTCATGATGATATA
TTTTTATCTTGTGCAATGTAACATCAGAGATTTTGAGACACAACGTGGCTTTCCCCCG
CCGCTCTAGAACTAGTGGATCCAAATAAAACGAAAGGCTCAGTCGAAAGACTGGGC
CTTTCGTTTTATCTGTTGTTTGTCGCATTATACGAGACGTCCAGGTTGGGATACCTGA
AACAAAACCCATCGTACGGCCAAGGAAGTCTCCAATAACTGTGATCCACCACAAGC
GCCAGGGTTTTCCCAGTCACGACGTTGTAAAACGACGGCCAGTCATGCATAATCCGC
ACGCATCTGGAATAAGGAAGTGCCATTCCGCCTGACCT
pLD20
(SEQ ID NO: 162)
TTAATTAATTCCGCTTAATGGAGTCCAAAAAGACCAACCTCTGCGCCTCGATCGACG
TGACCACAACCGCCGAGTTCCTTTCGCTCATCGACAAGCTCGGTCCCCACATCTGTC
TCGTGAAGACGCACATCGATATCATCTCAGACTTCAGCTACGAGGGCACGATTGAGC
CGTTGCTTGTGCTTGCAGAGCGCCACGGGTTCTTGATATTCGAGGACAGGAAGTTTG
CTGATATCGGAAACACCGTGATGTTGCAGTACACCTCGGGGGTATACCGGATCGCG
GCGTGGAGTGACATCACGAACGCGCACGGAGTGACTGGGAAGGGCGTCGTTGAAGG
GTTGAAACGCGGTGCGGAGGGGGTAGAAAAGGAAAGGGGCGTGTTGATGTTGGCG
GAGTTGTCGAGTAAAGGCTCGTTGGCGCATGGTGAATATACCCGTGAGACGATCGA
GATTGCGAAGAGTGATCGGGAGTTCGTGATTGGGTTCATCGCGCAGCGGGACATGG
GGGGTAGAGAAGAAGGGTTTGATTGGATCATCATGACGCCTGGTGTGGGGTTGGAT
GATAAAGGCGATGCGTTGGGCCAGCAGTATAGGACTGTTGATGAGGTGGTTCTGAC
TGGTACCGATGTGATTATTGTCGGGAGAGGGTTGTTTGGAAAAGGAAGAGACCCTG
AGGTGGAGGGAAAGAGATACAGGGATGCTGGATGGAAGGCATACTTGAAGAGAAC
TGGTCAGTTAGAATAAATATTGTAATAAATAGGTCTATATACATACACTAAGCTTCT
AGGACGTCATTGTAGTCTTCGAAGTTGTCTGCTAGTTTAGTTCTCATGATTTCGAAAA
CCAATAACGCAATGGATGTAGCAGGGATGGTGGTTAGTGCGTTCCTGACAAACCCA
GAGTACGCCGCCTCAAACCACGTCACATTCGCCCTTTGCTTCATCCGCATCACTTGCT
TGAAGGTATCCACGTACGAGTTGTAATACACCTTGAAGAACGGCTTCGTCTAGTTCG
GCATGGCAGATCATCATGCCTGCAGGAGCTCCAATTGTAATATTTCGGGAGAAATAT
CGTTGGGGTAAAACAACAGAGAGAGAGAGGGAGAGATGGTTCTGGTAGAATTATAA
TCTGGTTGTTGCAAATGCTACTGATCGACTCTGGCAATGTCTGTAGCTCGCTAGTTGT
ATGCAACTTAGGTGTTATGCATACACACGGTTATTCGGTTGAATTGTGGAGTAAAAA
TTGTCTGAGTTGTGTCTTAGCTACTGGCTGGCCCCCCGCGAAAGATAATCAAAATTA
CACTTGTGAATTTTTGCACACACACCGATTAACATTTCCCTTTTTTGTCCACCGATAC
ACGCTTGCCTCTTCTTATTTTCTCTGTGCTTCCCCCTCCTGTGACTTTTTCCACCATTG
ATATAAAATCAACTCCATTTCCCTAAAATCTCCCCAGATTCTAAAAACAACTTCTTCT
CTTCTGCTTTTCCTTATTTTTGTTATATTTATTTACCATCCCTTATTTTGAATAGTTATT
CCCCACTAACATTGTTCAAATCTTCACGACataATGAAGTGTTCTACGTTTAGTTTTTG
GTTTGTTTGTAAAATTATATTCTTCTTTTTTTCCTTCAACATTCAGACATCAATCGCCA
ACCCAAGGGAAAACTTCCTTAAGTGTTTTCTGCAGTACATCCCTAACAATGCAACAA
ACCTCAAGTTGGTGTACACTCAAAACAATCCACTCTATATGAGCGTGCTTAATAGCA
CAATCCACAACTTGCGCTTCACGTCAGATACTACGCCTAAGCCACTAGTGATCGTTA
CACCATCACACGTCAGCCATATTCAAGGAACGATCCTATGTCTGAAAAAGGTCGGGT
TGCAAATCAGGACTCGATCAGGAGGGCACGATAGTGAGGGAATGAGTTACATCTCG
CAAGTACCCTTCGTGATAGTTGACTTGCGAAATATGCGGTCTATTAAAATTGACGTA
CATAGCCAGACCGCCTGGGTTGAAGCAGGGGCAACCTTGGGTGAAGTTTATTACTG
GGTCAATGAAAAAAACGAAAACCTAAGTCTTGCTGCTGGATATTGCCCCACCGTTTG
CGCGGGTGGTCATTTTGGAGGCGGCGGATATGGTCCGTTGATGAGAAATTATGGACT
TGCAGCAGACAATATTATAGATGCCCACTTGGTGAACGTTCATGGAAAGGTCTTGGA
CCGTAAGTCCATGGGTGAAGATCTTTTCTGGGCCTTGAGAGGTGGTGGAGCGGAATC
GTTTGGCATCATCGTTGCCTGGAAAATTAGGTTGGTTGCGGTCCCGAAGAGTACAAT
GTTCTCCGTGAAGAAGATTATGGAAATACATGAGCTTGTCAAGTTAGTTAACAAGTG
GCAAAATATCGCTTATAAGTATGATAAAGACTTGCTTTTGATGACTCATTTTATTACG
CGAAACATAACCGATAACCAGGGCAAGAACAAGACTGCTATTCACACGTACTTCTC
CTCTGTATTTCTTGGAGGAGTAGACTCCTTAGTTGACTTGATGAACAAGAGTTTCCC
AGAATTGGGGATTAAGAAGACAGATTGCAGACAATTATCGTGGATAGATACAATCA
TATTCTATAGCGGTGTCGTCAATTACGATACTGATAATTTTAATAAAGAAATCCTCCT
AGATCGTTCAGCTGGGCAAAACGGGGCATTCAAAATTAAATTGGATTATGTGAAGA
AACCAATTCCAGAGCTGGTGTTTGTTCAGATATTGGAAAAACTTTACGAAGAAGACA
TTGGCGCAGGTATGTACGCTTTGTATCCATATGGAGGCATTATGGACGAGATCTCAG
AGCTGGCGATCCCCTTCCCGCACAGAGCTGGGATACTCTACGAGCTATGGTACATCT
GCTCTTGGGAGAAACAAGAAGACAACGAGAAACATCTCAATTGGATTCGGAACATA
TACAACTTTATGACCCCATACGTATCAAAAAACCCGCGCTTAGCATACTTGAATTAC
AGAGACTTAGATATCGGTATCAATGATCCTAAGAATCCTAACAATTACACCCAAGCC
CGTATTTGGGGTGAGAAATATTTCGGCAAGAATTTTGACAGATTAGTTAAGGTCAAA
ACACTCGTGGACCCCAACAACTTTTTCCGAAACGAGCAGTCGATTCCACCACTACCC
AGGCATAGACACTGATAAGAGTGACTCTTTTGATAAGAGTCGCAAATTTGATTTCAT
AAGTATATATTCATTATGTAAAGTAGTAAATGGAAAATTCATTAAAAAAAAAGCAA
ATTTCCGTTGTATGCATACTCCGAACACAAAACTAGCCCCGGAAAAACCCTTAGTTG
ATAGTTGCGAATTTAGGTCGACCATATGCGACGGGTACAACGAGAATTGTATTGAAT
TGATCAAGAACATGATCTTGGTGTTACAGAACATCAAGTTCTTGGACCAGACTGAGA
ATGCACAGATATACAAGGCGTCATGTGATAAAATGGATGAGATTTATCCACAATTG
AAGAAAGAGTTTATGGAAAGTGGTCAACCAGAAGCTAAACAGGAAGAAGCAAACG
AAGAGGTGAAACAAGAAGAAGAAGGTAAATAAGTATTTTGTATTATATAACAAACA
AAGTAAGGAATACAGATTTATACAATAAATTGCCATACTAGTCACGTGAGATATCTC
ATCCATTCCCCAACTCCCAAGAAAATAAAAAAGTGAAAAATAAAATCAAACCCAAA
GATCAACCTCCCCATCATCATCGTCATCAAACCCCCAGCTCAATTCGCAATGGTTAG
CACAAAAACATACACAGAAAGGGCATCAGCACACCCCTCCAAGGTTGCCCAACGTT
TATTAATTAAAGGCTAGGTGGAGGCTCAGTGATGATAAGTCTGCGATGGTGGATGC
ATGTGTCATGGTCATAGCTGTTTCCTGTGTGAAATTGTTATCCGCTCAGAGGGCACA
ATCCTATTCCGCGCTATCCGACAATCTCCAAGACATTAGGTGGAGTTCAGTTCGGCG
TATGGCATATGTCGCTGGAAAGAACATGTGAGCAAAAGGCCAGCAAAAGGCCAGGA
ACCGTAAAAAGGCCGCGTTGCTGGCGTTTTTCCATAGGCTCCGCCCCCCTGACGAGC
ATCACAAAAATCGACGCTCAAGTCAGAGGTGGCGAAACCCGACAGGACTATAAAGA
TACCAGGCGTTTCCCCCTGGAAGCTCCCTCGTGCGCTCTCCTGTTCCGACCCTGCCGC
TTACCGGATACCTGTCCGCCTTTCTCCCTTCGGGAAGCGTGGCGCTTTCTCATAGCTC
ACGCTGTAGGTATCTCAGTTCGGTGTAGGTCGTTCGCTCCAAGCTGGGCTGTGTGCA
CGAACCCCCCGTTCAGCCCGACCGCTGCGCCTTATCCGGTAACTATCGTCTTGAGTC
CAACCCGGTAAGACACGACTTATCGCCACTGGCAGCAGCCACTGGTAACAGGATTA
GCAGAGCGAGGTATGTAGGCGGTGCTACAGAGTTCTTGAAGTGGTGGCCTAACTAC
GGCTACACTAGAAGAACAGTATTTGGTATCTGCGCTCTGCTGAAGCCAGTTACCTTC
GGAAAAAGAGTTGGTAGCTCTTGATCCGGCAAACAAACCACCGCTGGTAGCGGTGG
TTTTTTTGTTTGCAAGCAGCAGATTACGCGCAGAAAAAAAGGATCTCAAGAAGATCC
TTTGATCTTTTCTACGGGGTCTGACGCTCTATTCAACAAAGCCGCCGTCCCGTCAAGT
CAGCGTAAATGGGTAGGGGGCTTCAAATCGTCCGCTCTGCCAGTGTTACAACCAATT
AACAAATTCTGATTAGAAAAACTCATCGAGCATCAAATGAAACTGCAATTTATTCAT
ATCAGGATTATCAATACCATATTTTTGAAAAAGCCGTTTCTGTAATGAAGGAGAAAA
CTCACCGAGGCAGTTCCATAGGATGGCAAGATCCTGGTATCGGTCTGCGATTCCGAC
TCGTCCAACATCAATACAACCTATTAATTTCCCCTCGTCAAAAATAAGGTTATCAAG
TGAGAAATCACCATGAGTGACGACTGAATCCGGTGAGAATGGCAAAAGCTTATGCA
TTTCTTTCCAGACTTGTTCAACAGGCCAGCCATTACGCTCGTCATCAAAATCACTCGC
ATCAACCAAACCGTTATTCATTCGTGATTGCGCCTGAGCGAGACGAAATACGCGATC
GCTGTTAAAAGGACAATTACAAACAGGAATCGAATGCAACCGGCGCAGGAACACTG
CCAGCGCATCAACAATATTTTCACCTGAATCAGGATATTCTTCTAATACCTGGAATG
CTGTTTTCCCGGGGATCGCAGTGGTGAGTAACCATGCATCATCAGGAGTACGGATAA
AATGCTTGATGGTCGGAAGAGGCATAAATTCCGTCAGCCAGTTTAGTCTGACCATCT
CATCTGTAACATCATTGGCAACGCTACCTTTGCCATGTTTCAGAAACAACTCTGGCG
CATCGGGCTTCCCATACAATCGATAGATTGTCGCACCTGATTGCCCGACATTATCGC
GAGCCCATTTATACCCATATAAATCAGCATCCATGTTGGAATTTAATCGCGGCCTCG
AGCAAGACGTTTCCCGTTGAATATGGCTCATAACACCCCTTGTATTACTGTTTATGTA
AGCAGACAGTTTTATTGTTCATGATGATATATTTTTATCTTGTGCAATGTAACATCAG
AGATTTTGAGACACAACGTGGCTTTCCCCCGCCGCTCTAGAACTAGTGGATCCAAAT
AAAACGAAAGGCTCAGTCGAAAGACTGGGCCTTTCGTTTTATCTGTTGTTTGTCGCA
TTATACGAGACGTCCAGGTTGGGATACCTGAAACAAAACCCATCGTACGGCCAAGG
AAGTCTCCAATAACTGTGATCCACCACAAGCGCCAGGGTTTTCCCAGTCACGACGTT
GTAAAACGACGGCCAGTCATGCATAATCCGCACGCATCTGGAATAAGGAAGTGCCA
TTCCGCCTGACCT
pLD22
(SEQ ID NO: 163)
TTAATTAATTCCGCTTAATGGAGTCCAAAAAGACCAACCTCTGCGCCTCGATCGACG
TGACCACAACCGCCGAGTTCCTTTCGCTCATCGACAAGCTCGGTCCCCACATCTGTC
TCGTGAAGACGCACATCGATATCATCTCAGACTTCAGCTACGAGGGCACGATTGAGC
CGTTGCTTGTGCTTGCAGAGCGCCACGGGTTCTTGATATTCGAGGACAGGAAGTTTG
CTGATATCGGAAACACCGTGATGTTGCAGTACACCTCGGGGGTATACCGGATCGCG
GCGTGGAGTGACATCACGAACGCGCACGGAGTGACTGGGAAGGGCGTCGTTGAAGG
GTTGAAACGCGGTGCGGAGGGGGTAGAAAAGGAAAGGGGCGTGTTGATGTTGGCG
GAGTTGTCGAGTAAAGGCTCGTTGGCGCATGGTGAATATACCCGTGAGACGATCGA
GATTGCGAAGAGTGATCGGGAGTTCGTGATTGGGTTCATCGCGCAGCGGGACATGG
GGGGTAGAGAAGAAGGGTTTGATTGGATCATCATGACGCCTGGTGTGGGGTTGGAT
GATAAAGGCGATGCGTTGGGCCAGCAGTATAGGACTGTTGATGAGGTGGTTCTGAC
TGGTACCGATGTGATTATTGTCGGGAGAGGGTTGTTTGGAAAAGGAAGAGACCCTG
AGGTGGAGGGAAAGAGATACAGGGATGCTGGATGGAAGGCATACTTGAAGAGAAC
TGGTCAGTTAGAATAAATATTGTAATAAATAGGTCTATATACATACACTAAGCTTCT
AGGACGTCATTGTAGTCTTCGAAGTTGTCTGCTAGTTTAGTTCTCATGATTTCGAAAA
CCAATAACGCAATGGATGTAGCAGGGATGGTGGTTAGTGCGTTCCTGACAAACCCA
GAGTACGCCGCCTCAAACCACGTCACATTCGCCCTTTGCTTCATCCGCATCACTTGCT
TGAAGGTATCCACGTACGAGTTGTAATACACCTTGAAGAACGGCTTCGTCTAGTTCG
GCATGGCAGATCATCATGCCTGCAGGAGCTCCAATTGTAATATTTCGGGAGAAATAT
CGTTGGGGTAAAACAACAGAGAGAGAGAGGGAGAGATGGTTCTGGTAGAATTATAA
TCTGGTTGTTGCAAATGCTACTGATCGACTCTGGCAATGTCTGTAGCTCGCTAGTTGT
ATGCAACTTAGGTGTTATGCATACACACGGTTATTCGGTTGAATTGTGGAGTAAAAA
TTGTCTGAGTTGTGTCTTAGCTACTGGCTGGCCCCCCGCGAAAGATAATCAAAATTA
CACTTGTGAATTTTTGCACACACACCGATTAACATTTCCCTTTTTTGTCCACCGATAC
ACGCTTGCCTCTTCTTATTTTCTCTGTGCTTCCCCCTCCTGTGACTTTTTCCACCATTG
ATATAAAATCAACTCCATTTCCCTAAAATCTCCCCAGATTCTAAAAACAACTTCTTCT
CTTCTGCTTTTCCTTATTTTTGTTATATTTATTTACCATCCCTTATTTTGAATAGTTATT
CCCCACTAACATTGTTCAAATCTTCACGACataATGACAGAAGTAGTAGATAGAGCAA
GTTCCCCAGCAAGTCCAGGATCTACGACCGCCGCCGCAGACGGTGCTAAGGTGGCG
GTGGAGCCACGCGTAGATGTAGCGGCCCTTGGCGAGCAGTTGCTAGGGCGATGGGC
TGACATCAGATTGCACGCACGAGACTTAGCAGGCCGCGAAGTGGTCCAAAAGGTTG
AAGGACTTACGCACACTGAGCATCGGAGTAGAGTCTTTGGACAGTTGAAGTACTTG
GTAGACAACAATGCTGTTCACAGAGCTTTTCCCTCCAGGCTAGGTGGATCAGATGAC
CATGGCGGTAATATAGCTGGATTCGAGGAATTAGTTACTGCTGATCCATCATTGCAA
ATAAAGGCCGGCGTTCAGTGGGGTTTGTTTGGTTCTGCAGTGATGCACTTGGGAACC
CGTGAACATCATGACAAGTGGTTGCCAGGTATTATGTCGTTAGAAATACCGGGGTGT
TTCGCGATGACAGAAACCGGGCACGGTAGCGACGTGGCCTCTATTGCTACAACAGC
AACTTATGATGAGGAAACCCAAGAGTTTGTTATTGATACCCCGTTCAGAGCCGCTTG
GAAAGATTATATCGGTAATGCAGCGAACGATGGTTTGGCGGCAGTTGTTTTCGCACA
ATTAATCACGAGGAAAGTGAACCATGGTGTACACGCCTTTTACGTGGATCTCAGAGA
TCCTGCGACTGGAGACTTCCTACCCGGAATAGGAGGAGAGGACGATGGAATCAAGG
GGGGATTGAATGGCATTGACAACGGTAGACTACATTTTACGAACGTACGCATTCCTA
GAACTAATCTTCTTAACAGATATGGCGATGTGGCGGTCGACGGCACATACCTGTCGA
CCATCGAATCACCAGGGCGCCGGTTCTTTACGATGCTTGGTACTCTAGTCCAGGGTA
GAGTTAGTCTCGATGGAGCAGCTGTCGCTGCACTGAAGGTCGCATTGCAAAGTGCA
ATTCACTACGCTGCGGAGAGGAGACAATTTAATGCGACTTCACCTACTGAAGAAGA
GGTCCTTCTTGATTATCAGAGGCATCAAAGGAGACTCTTTACACGACTTGCAACGAC
GTACGCCGCATCTTTCGCCCACGAGCAGCTATTGCAAAAGTTCGATGATGTCTTTTC
AGGGGCACATGATACCGACGCCGACCGGCAGGACTTGGAAACCCTAGCCGCCGCTT
TGAAGCCATTGAGCACATGGCATGCACTTGACACGTTACAAGAATGCAGAGAGGCC
TGTGGGGGGGCCGGATTTTTGATAGAAAACCGTTTCGCGAGCTTGCGTGCTGACTTG
GACGTTTACGTCACATTCGAGGGTGATAACACAGTTTTATTGCAATTGGTTGCTAAA
CGGCTCTTGGCAGACTACGCAAAAGAGTTCAGAGGGGCCAACTTCGGCGTTCTTGCC
AGGTATGTGGTTGACCAAGCCGCGGGAGTGGCGCTCCACCGAACAGGACTAAGGCA
AGTCGCTCAATTTGTTGCAGACAGCGGGTCCGTTCAGAAGTCGGCTCTTGCGCTTCG
CGATGAAGAGGGTCAACGAACATTGTTAACGGACAGAGTACAGAGCATGGTTGCCG
AAGTGGGGGCTGCCTTGAAAGGCGCAGGCAAATTACCCCAACATCAAGCAGCTGCA
TTGTTCAACCAACACCAGAACGAACTTATTGAGGCTGCCCAGGCCCATGCAGAACTC
CTCCAATGGGAGGCATTTACAGAAGCTCTCGCTAAAGTCGACGATGCTGGTACAAA
GGAAGTGCTTACTCGATTGCGAGATCTCTTTGGTTTGTCCTTGATTGAAAAACACTTG
CTGTGGTATCTTATGAATGGACGTTTGTCCATGCAAAGAGGCAGGACAGTTGGAACT
TACATTAATCGTTTACTTGTCAAGATCCGTCCACACGCACTAGACTTGGTTGATGCCT
TCGGTTACGGCGCGGAGCATTTGCGTGCTGCTATCGCCACCGGAGCGGAAGCAACC
CGACAGGATGAAGCCCGAACGTATTTTAGACAACAACGGGCATCGGGACTGGCCCC
GGCCGATGAAAAGACCTTACTCGCTATCAAAGCTGGTAAATCAAGAGGGCGAAGGG
CAAAGCTATGATAAGAGTGACTCTTTTGATAAGAGTCGCAAATTTGATTTCATAAGT
ATATATTCATTATGTAAAGTAGTAAATGGAAAATTCATTAAAAAAAAAGCAAATTTC
CGTTGTATGCATACTCCGAACACAAAACTAGCCCCGGAAAAACCCTTAGTTGATAGT
TGCGAATTTAGGTCGACCATATGCGACGGGTACAACGAGAATTGTATTGAATTGATC
AAGAACATGATCTTGGTGTTACAGAACATCAAGTTCTTGGACCAGACTGAGAATGC
ACAGATATACAAGGCGTCATGTGATAAAATGGATGAGATTTATCCACAATTGAAGA
AAGAGTTTATGGAAAGTGGTCAACCAGAAGCTAAACAGGAAGAAGCAAACGAAGA
GGTGAAACAAGAAGAAGAAGGTAAATAAGTATTTTGTATTATATAACAAACAAAGT
AAGGAATACAGATTTATACAATAAATTGCCATACTAGTCACGTGAGATATCTCATCC
ATTCCCCAACTCCCAAGAAAATAAAAAAGTGAAAAATAAAATCAAACCCAAAGATC
AACCTCCCCATCATCATCGTCATCAAACCCCCAGCTCAATTCGCAATGGTTAGCACA
AAAACATACACAGAAAGGGCATCAGCACACCCCTCCAAGGTTGCCCAACGTTTATT
AATTAAAGGCTAGGTGGAGGCTCAGTGATGATAAGTCTGCGATGGTGGATGCATGT
GTCATGGTCATAGCTGTTTCCTGTGTGAAATTGTTATCCGCTCAGAGGGCACAATCC
TATTCCGCGCTATCCGACAATCTCCAAGACATTAGGTGGAGTTCAGTTCGGCGTATG
GCATATGTCGCTGGAAAGAACATGTGAGCAAAAGGCCAGCAAAAGGCCAGGAACC
GTAAAAAGGCCGCGTTGCTGGCGTTTTTCCATAGGCTCCGCCCCCCTGACGAGCATC
ACAAAAATCGACGCTCAAGTCAGAGGTGGCGAAACCCGACAGGACTATAAAGATAC
CAGGCGTTTCCCCCTGGAAGCTCCCTCGTGCGCTCTCCTGTTCCGACCCTGCCGCTTA
CCGGATACCTGTCCGCCTTTCTCCCTTCGGGAAGCGTGGCGCTTTCTCATAGCTCACG
CTGTAGGTATCTCAGTTCGGTGTAGGTCGTTCGCTCCAAGCTGGGCTGTGTGCACGA
ACCCCCCGTTCAGCCCGACCGCTGCGCCTTATCCGGTAACTATCGTCTTGAGTCCAA
CCCGGTAAGACACGACTTATCGCCACTGGCAGCAGCCACTGGTAACAGGATTAGCA
GAGCGAGGTATGTAGGCGGTGCTACAGAGTTCTTGAAGTGGTGGCCTAACTACGGC
TACACTAGAAGAACAGTATTTGGTATCTGCGCTCTGCTGAAGCCAGTTACCTTCGGA
AAAAGAGTTGGTAGCTCTTGATCCGGCAAACAAACCACCGCTGGTAGCGGTGGTTTT
TTTGTTTGCAAGCAGCAGATTACGCGCAGAAAAAAAGGATCTCAAGAAGATCCTTT
GATCTTTTCTACGGGGTCTGACGCTCTATTCAACAAAGCCGCCGTCCCGTCAAGTCA
GCGTAAATGGGTAGGGGGCTTCAAATCGTCCGCTCTGCCAGTGTTACAACCAATTAA
CAAATTCTGATTAGAAAAACTCATCGAGCATCAAATGAAACTGCAATTTATTCATAT
CAGGATTATCAATACCATATTTTTGAAAAAGCCGTTTCTGTAATGAAGGAGAAAACT
CACCGAGGCAGTTCCATAGGATGGCAAGATCCTGGTATCGGTCTGCGATTCCGACTC
GTCCAACATCAATACAACCTATTAATTTCCCCTCGTCAAAAATAAGGTTATCAAGTG
AGAAATCACCATGAGTGACGACTGAATCCGGTGAGAATGGCAAAAGCTTATGCATT
TCTTTCCAGACTTGTTCAACAGGCCAGCCATTACGCTCGTCATCAAAATCACTCGCA
TCAACCAAACCGTTATTCATTCGTGATTGCGCCTGAGCGAGACGAAATACGCGATCG
CTGTTAAAAGGACAATTACAAACAGGAATCGAATGCAACCGGCGCAGGAACACTGC
CAGCGCATCAACAATATTTTCACCTGAATCAGGATATTCTTCTAATACCTGGAATGC
TGTTTTCCCGGGGATCGCAGTGGTGAGTAACCATGCATCATCAGGAGTACGGATAAA
ATGCTTGATGGTCGGAAGAGGCATAAATTCCGTCAGCCAGTTTAGTCTGACCATCTC
ATCTGTAACATCATTGGCAACGCTACCTTTGCCATGTTTCAGAAACAACTCTGGCGC
ATCGGGCTTCCCATACAATCGATAGATTGTCGCACCTGATTGCCCGACATTATCGCG
AGCCCATTTATACCCATATAAATCAGCATCCATGTTGGAATTTAATCGCGGCCTCGA
GCAAGACGTTTCCCGTTGAATATGGCTCATAACACCCCTTGTATTACTGTTTATGTAA
GCAGACAGTTTTATTGTTCATGATGATATATTTTTATCTTGTGCAATGTAACATCAGA
GATTTTGAGACACAACGTGGCTTTCCCCCGCCGCTCTAGAACTAGTGGATCCAAATA
AAACGAAAGGCTCAGTCGAAAGACTGGGCCTTTCGTTTTATCTGTTGTTTGTCGCAT
TATACGAGACGTCCAGGTTGGGATACCTGAAACAAAACCCATCGTACGGCCAAGGA
AGTCTCCAATAACTGTGATCCACCACAAGCGCCAGGGTTTTCCCAGTCACGACGTTG
TAAAACGACGGCCAGTCATGCATAATCCGCACGCATCTGGAATAAGGAAGTGCCAT
TCCGCCTGACCT
pLD24
(SEQ ID NO: 164)
TTAATTAATTCCGCTTAATGGAGTCCAAAAAGACCAACCTCTGCGCCTCGATCGACG
TGACCACAACCGCCGAGTTCCTTTCGCTCATCGACAAGCTCGGTCCCCACATCTGTC
TCGTGAAGACGCACATCGATATCATCTCAGACTTCAGCTACGAGGGCACGATTGAGC
CGTTGCTTGTGCTTGCAGAGCGCCACGGGTTCTTGATATTCGAGGACAGGAAGTTTG
CTGATATCGGAAACACCGTGATGTTGCAGTACACCTCGGGGGTATACCGGATCGCG
GCGTGGAGTGACATCACGAACGCGCACGGAGTGACTGGGAAGGGCGTCGTTGAAGG
GTTGAAACGCGGTGCGGAGGGGGTAGAAAAGGAAAGGGGCGTGTTGATGTTGGCG
GAGTTGTCGAGTAAAGGCTCGTTGGCGCATGGTGAATATACCCGTGAGACGATCGA
GATTGCGAAGAGTGATCGGGAGTTCGTGATTGGGTTCATCGCGCAGCGGGACATGG
GGGGTAGAGAAGAAGGGTTTGATTGGATCATCATGACGCCTGGTGTGGGGTTGGAT
GATAAAGGCGATGCGTTGGGCCAGCAGTATAGGACTGTTGATGAGGTGGTTCTGAC
TGGTACCGATGTGATTATTGTCGGGAGAGGGTTGTTTGGAAAAGGAAGAGACCCTG
AGGTGGAGGGAAAGAGATACAGGGATGCTGGATGGAAGGCATACTTGAAGAGAAC
TGGTCAGTTAGAATAAATATTGTAATAAATAGGTCTATATACATACACTAAGCTTCT
AGGACGTCATTGTAGTCTTCGAAGTTGTCTGCTAGTTTAGTTCTCATGATTTCGAAAA
CCAATAACGCAATGGATGTAGCAGGGATGGTGGTTAGTGCGTTCCTGACAAACCCA
GAGTACGCCGCCTCAAACCACGTCACATTCGCCCTTTGCTTCATCCGCATCACTTGCT
TGAAGGTATCCACGTACGAGTTGTAATACACCTTGAAGAACGGCTTCGTCTAGTTCG
GCATGGCAGATCATCATGCCTGCAGGAGCTCCAATTGTAATATTTCGGGAGAAATAT
CGTTGGGGTAAAACAACAGAGAGAGAGAGGGAGAGATGGTTCTGGTAGAATTATAA
TCTGGTTGTTGCAAATGCTACTGATCGACTCTGGCAATGTCTGTAGCTCGCTAGTTGT
ATGCAACTTAGGTGTTATGCATACACACGGTTATTCGGTTGAATTGTGGAGTAAAAA
TTGTCTGAGTTGTGTCTTAGCTACTGGCTGGCCCCCCGCGAAAGATAATCAAAATTA
CACTTGTGAATTTTTGCACACACACCGATTAACATTTCCCTTTTTTGTCCACCGATAC
ACGCTTGCCTCTTCTTATTTTCTCTGTGCTTCCCCCTCCTGTGACTTTTTCCACCATTG
ATATAAAATCAACTCCATTTCCCTAAAATCTCCCCAGATTCTAAAAACAACTTCTTCT
CTTCTGCTTTTCCTTATTTTTGTTATATTTATTTACCATCCCTTATTTTGAATAGTTATT
CCCCACTAACATTGTTCAAATCTTCACGACataATGAATCATTTAAGAGCAGAAGGAC
CCGCATCAGTGTTAGCGATAGGTACAGCTAACCCAGAGAATATCTTAATCCAAGATG
AATTTCCTGACTACTATTTCCGTGTTACTAAATCGGAACATATGACTCAACTTAAAG
AGAAGTTCCGGAAAATCTGCGATAAATCCATGATCCGAAAGAGAAACTGTTTCCTTA
ACGAAGAACATCTCAAGCAAAACCCGAGGTTGGTAGAGCACGAAATGCAGACCTTG
GATGCTAGGCAGGACATGTTGGTGGTCGAAGTGCCAAAACTCGGCAAGGACGCGTG
CGCTAAGGCAATCAAGGAGTGGGGTCAACCGAAGTCTAAAATCACGCATCTAATAT
TTACATCTGCACTGACAACCGACATGCCGGGTGCCGATTATCACTGCGCCAAGCTAC
TTGGATTGAGTCCACTGGTTAAGAGAGTTATGATGTATCAATTGGGGTGTTACGGAG
GGGGCACAGTCCTCAGAATTGCTAAGGATATTGCGGAAAATAACAAGGGCGCGAGG
GTCCTTGCTGTATGTTGTGATATTATGGCCTGTTTGTTTCGCGGGCCCTCGGATTCAG
ATTTGGAATTGCTTGTCGGACAGGCAATTTTTGGTGACGGGGCCGCAGCAGTCATAG
TGGGAGCCGAACCAGACGAAAGCGTGGGTGAAAGACCAATCTTTGAGTTGGTTCTG
ACCGGACAAACGATCTTACCTAACTCGGAAGGTACGATTGGAGGACATATTAGAGA
AGCCGGCCTAATTTTCGATCTTCACAAAGACGTTCCAATGTTAATCTCCAATAACAT
AGAAAAGTGCTTGATAGAAGCATTTACTCCCATTGGTATTAGTGACTGGAACAGCAT
TTTCTGGATCACCCACCCTGGAGGAAAAGCTATACTCGATAAGGTTGAAGAGAAAC
TCGACTTGAAAAAGGAGAAATTCGTTGACTCACGACATGTGTTATCAGAGCACGGG
AATATGAGTTCATCCACAGTCTTGTTCGTAATGGATGAATTGCGAAAACGCTCTCTT
GAGGAGGGAAAGAGCACAACCGGTGACGGGTTTGAGTGGGGCGTGCTATTCGGTTT
TGGCCCAGGTTTGACTGTCGAGCGGGTTGTTGTTCGTAGTGTACCAATTAAGTACGG
AAGAAGGGCAAAGTTGTGATAAGAGTGACTCTTTTGATAAGAGTCGCAAATTTGATT
TCATAAGTATATATTCATTATGTAAAGTAGTAAATGGAAAATTCATTAAAAAAAAAG
CAAATTTCCGTTGTATGCATACTCCGAACACAAAACTAGCCCCGGAAAAACCCTTAG
TTGATAGTTGCGAATTTAGGTCGACCATATGCGACGGGTACAACGAGAATTGTATTG
AATTGATCAAGAACATGATCTTGGTGTTACAGAACATCAAGTTCTTGGACCAGACTG
AGAATGCACAGATATACAAGGCGTCATGTGATAAAATGGATGAGATTTATCCACAA
TTGAAGAAAGAGTTTATGGAAAGTGGTCAACCAGAAGCTAAACAGGAAGAAGCAA
ACGAAGAGGTGAAACAAGAAGAAGAAGGTAAATAAGTATTTTGTATTATATAACAA
ACAAAGTAAGGAATACAGATTTATACAATAAATTGCCATACTAGTCACGTGAGATA
TCTCATCCATTCCCCAACTCCCAAGAAAATAAAAAAGTGAAAAATAAAATCAAACC
CAAAGATCAACCTCCCCATCATCATCGTCATCAAACCCCCAGCTCAATTCGCAATGG
TTAGCACAAAAACATACACAGAAAGGGCATCAGCACACCCCTCCAAGGTTGCCCAA
CGTTTATTAATTAAAGGCTAGGTGGAGGCTCAGTGATGATAAGTCTGCGATGGTGGA
TGCATGTGTCATGGTCATAGCTGTTTCCTGTGTGAAATTGTTATCCGCTCAGAGGGC
ACAATCCTATTCCGCGCTATCCGACAATCTCCAAGACATTAGGTGGAGTTCAGTTCG
GCGTATGGCATATGTCGCTGGAAAGAACATGTGAGCAAAAGGCCAGCAAAAGGCCA
GGAACCGTAAAAAGGCCGCGTTGCTGGCGTTTTTCCATAGGCTCCGCCCCCCTGACG
AGCATCACAAAAATCGACGCTCAAGTCAGAGGTGGCGAAACCCGACAGGACTATAA
AGATACCAGGCGTTTCCCCCTGGAAGCTCCCTCGTGCGCTCTCCTGTTCCGACCCTG
CCGCTTACCGGATACCTGTCCGCCTTTCTCCCTTCGGGAAGCGTGGCGCTTTCTCATA
GCTCACGCTGTAGGTATCTCAGTTCGGTGTAGGTCGTTCGCTCCAAGCTGGGCTGTG
TGCACGAACCCCCCGTTCAGCCCGACCGCTGCGCCTTATCCGGTAACTATCGTCTTG
AGTCCAACCCGGTAAGACACGACTTATCGCCACTGGCAGCAGCCACTGGTAACAGG
ATTAGCAGAGCGAGGTATGTAGGCGGTGCTACAGAGTTCTTGAAGTGGTGGCCTAA
CTACGGCTACACTAGAAGAACAGTATTTGGTATCTGCGCTCTGCTGAAGCCAGTTAC
CTTCGGAAAAAGAGTTGGTAGCTCTTGATCCGGCAAACAAACCACCGCTGGTAGCG
GTGGTTTTTTTGTTTGCAAGCAGCAGATTACGCGCAGAAAAAAAGGATCTCAAGAA
GATCCTTTGATCTTTTCTACGGGGTCTGACGCTCTATTCAACAAAGCCGCCGTCCCGT
CAAGTCAGCGTAAATGGGTAGGGGGCTTCAAATCGTCCGCTCTGCCAGTGTTACAAC
CAATTAACAAATTCTGATTAGAAAAACTCATCGAGCATCAAATGAAACTGCAATTTA
TTCATATCAGGATTATCAATACCATATTTTTGAAAAAGCCGTTTCTGTAATGAAGGA
GAAAACTCACCGAGGCAGTTCCATAGGATGGCAAGATCCTGGTATCGGTCTGCGATT
CCGACTCGTCCAACATCAATACAACCTATTAATTTCCCCTCGTCAAAAATAAGGTTA
TCAAGTGAGAAATCACCATGAGTGACGACTGAATCCGGTGAGAATGGCAAAAGCTT
ATGCATTTCTTTCCAGACTTGTTCAACAGGCCAGCCATTACGCTCGTCATCAAAATC
ACTCGCATCAACCAAACCGTTATTCATTCGTGATTGCGCCTGAGCGAGACGAAATAC
GCGATCGCTGTTAAAAGGACAATTACAAACAGGAATCGAATGCAACCGGCGCAGGA
ACACTGCCAGCGCATCAACAATATTTTCACCTGAATCAGGATATTCTTCTAATACCT
GGAATGCTGTTTTCCCGGGGATCGCAGTGGTGAGTAACCATGCATCATCAGGAGTAC
GGATAAAATGCTTGATGGTCGGAAGAGGCATAAATTCCGTCAGCCAGTTTAGTCTGA
CCATCTCATCTGTAACATCATTGGCAACGCTACCTTTGCCATGTTTCAGAAACAACTC
TGGCGCATCGGGCTTCCCATACAATCGATAGATTGTCGCACCTGATTGCCCGACATT
ATCGCGAGCCCATTTATACCCATATAAATCAGCATCCATGTTGGAATTTAATCGCGG
CCTCGAGCAAGACGTTTCCCGTTGAATATGGCTCATAACACCCCTTGTATTACTGTTT
ATGTAAGCAGACAGTTTTATTGTTCATGATGATATATTTTTATCTTGTGCAATGTAAC
ATCAGAGATTTTGAGACACAACGTGGCTTTCCCCCGCCGCTCTAGAACTAGTGGATC
CAAATAAAACGAAAGGCTCAGTCGAAAGACTGGGCCTTTCGTTTTATCTGTTGTTTG
TCGCATTATACGAGACGTCCAGGTTGGGATACCTGAAACAAAACCCATCGTACGGC
CAAGGAAGTCTCCAATAACTGTGATCCACCACAAGCGCCAGGGTTTTCCCAGTCACG
ACGTTGTAAAACGACGGCCAGTCATGCATAATCCGCACGCATCTGGAATAAGGAAG
TGCCATTCCGCCTGACCT
pLD56
(SEQ ID NO: 165)
TTAATTAATTCCGCTTAATGGAGTCCAAAAAGACCAACCTCTGCGCCTCGATCGACG
TGACCACAACCGCCGAGTTCCTTTCGCTCATCGACAAGCTCGGTCCCCACATCTGTC
TCGTGAAGACGCACATCGATATCATCTCAGACTTCAGCTACGAGGGCACGATTGAGC
CGTTGCTTGTGCTTGCAGAGCGCCACGGGTTCTTGATATTCGAGGACAGGAAGTTTG
CTGATATCGGAAACACCGTGATGTTGCAGTACACCTCGGGGGTATACCGGATCGCG
GCGTGGAGTGACATCACGAACGCGCACGGAGTGACTGGGAAGGGCGTCGTTGAAGG
GTTGAAACGCGGTGCGGAGGGGGTAGAAAAGGAAAGGGGCGTGTTGATGTTGGCG
GAGTTGTCGAGTAAAGGCTCGTTGGCGCATGGTGAATATACCCGTGAGACGATCGA
GATTGCGAAGAGTGATCGGGAGTTCGTGATTGGGTTCATCGCGCAGCGGGACATGG
GGGGTAGAGAAGAAGGGTTTGATTGGATCATCATGACGCCTGGTGTGGGGTTGGAT
GATAAAGGCGATGCGTTGGGCCAGCAGTATAGGACTGTTGATGAGGTGGTTCTGAC
TGGTACCGATGTGATTATTGTCGGGAGAGGGTTGTTTGGAAAAGGAAGAGACCCTG
AGGTGGAGGGAAAGAGATACAGGGATGCTGGATGGAAGGCATACTTGAAGAGAAC
TGGTCAGTTAGAATAAATATTGTAATAAATAGGTCTATATACATACACTAAGCTTCT
AGGACGTCATTGTAGTCTTCGAAGTTGTCTGCTAGTTTAGTTCTCATGATTTCGAAAA
CCAATAACGCAATGGATGTAGCAGGGATGGTGGTTAGTGCGTTCCTGACAAACCCA
GAGTACGCCGCCTCAAACCACGTCACATTCGCCCTTTGCTTCATCCGCATCACTTGCT
TGAAGGTATCCACGTACGAGTTGTAATACACCTTGAAGAACGGCTTCGTCTAGTTCG
GCATGGCAGATCATCATGCCTGCAGGAGCTCCAATTGTAATATTTCGGGAGAAATAT
CGTTGGGGTAAAACAACAGAGAGAGAGAGGGAGAGATGGTTCTGGTAGAATTATAA
TCTGGTTGTTGCAAATGCTACTGATCGACTCTGGCAATGTCTGTAGCTCGCTAGTTGT
ATGCAACTTAGGTGTTATGCATACACACGGTTATTCGGTTGAATTGTGGAGTAAAAA
TTGTCTGAGTTGTGTCTTAGCTACTGGCTGGCCCCCCGCGAAAGATAATCAAAATTA
CACTTGTGAATTTTTGCACACACACCGATTAACATTTCCCTTTTTTGTCCACCGATAC
ACGCTTGCCTCTTCTTATTTTCTCTGTGCTTCCCCCTCCTGTGACTTTTTCCACCATTG
ATATAAAATCAACTCCATTTCCCTAAAATCTCCCCAGATTCTAAAAACAACTTCTTCT
CTTCTGCTTTTCCTTATTTTTGTTATATTTATTTACCATCCCTTATTTTGAATAGTTATT
CCCCACTAACATTGTTCAAATCTTCACGACataATGGGGTTGTCCTTAGTTTGTACGTT
CAGTTTCCAAACTAACTACCACACACTACTAAATCCGCACAACAAAAACCCGAAAA
ATTCATTGCTCTCCTATCAGCACCCAAAAACACCCATTATCAAGTCTAGTTACGACA
ACTTTCCATCAAAATACTGTCTAACGAAAAACTTTCATTTGTTGGGCTTAAATTCTCA
TAATCGTATTTCCAGTCAGTCCCGATCGATCAGGGCCGGGAGTGACCAAATTGAAGG
TTCTCCACATCATGAAAGTGACAATTCAATTGCTACGAAGATTTTAAACTTTGGGCA
TACATGCTGGAAGCTACAGCGACCGTATGTAGTTAAGGGGATGATCAGCATTGCCTG
CGGCCTATTCGGAAGGGAACTCTTCAATAATAGACATCTTTTTTCTTGGGGTTTAATG
TGGAAAGCTTTTTTCGCTTTGGTTCCTATCCTTAGTTTTAACTTCTTCGCCGCTATTAT
GAATCAAATTTACGATGTTGACATCGACCGTATTAACAAACCCGATCTCCCCCTTGT
TTCAGGCGAGATGTCCATTGAAACGGCATGGATTTTGTCCATCATTGTTGCGCTTACT
GGCTTGATTGTTACCATTAAGCTTAAAAGCGCTCCCTTGTTCGTTTTTATATACATTT
TCGGCATTTTTGCCGGATTCGCATACAGTGTCCCGCCTATACGTTGGAAACAATATC
CATTCACGAACTTCTTGATCACGATCTCATCACATGTTGGATTGGCCTTTACGTCCTA
CAGTGCTACCACATCTGCCCTTGGATTGCCTTTCGTTTGGAGGCCTGCCTTCTCGTTT
ATCATTGCATTTATGACAGTGATGGGAATGACTATCGCATTTGCTAAAGATATCAGC
GACATAGAGGGCGATGCAAAATATGGGGTGAGTACTGTTGCGACGAAGTTGGGCGC
CCGAAATATGACCTTCGTTGTTTCCGGCGTTCTTTTACTTAACTATTTAGTATCGATT
AGCATCGGGATCATCTGGCCACAGGTGTTTAAATCAAATATTATGATCTTGTCGCAT
GCCATCCTAGCTTTCTGTCTTATATTTCAAACAAGAGAATTAGCCCTAGCGAACTAC
GCCTCAGCACCAAGTCGTCAGTTCTTCGAATTTATATGGCTACTCTACTACGCCGAA
TACTTCGTCTATGTCTTCATTTAGTAATAAGAGTGACTCTTTTGATAAGAGTCGCAAA
TTTGATTTCATAAGTATATATTCATTATGTAAAGTAGTAAATGGAAAATTCATTAAA
AAAAAAGCAAATTTCCGTTGTATGCATACTCCGAACACAAAACTAGCCCCGGAAAA
ACCCTTAGTTGATAGTTGCGAATTTAGGTCGACCATATGCGACGGGTACAACGAGAA
TTGTATTGAATTGATCAAGAACATGATCTTGGTGTTACAGAACATCAAGTTCTTGGA
CCAGACTGAGAATGCACAGATATACAAGGCGTCATGTGATAAAATGGATGAGATTT
ATCCACAATTGAAGAAAGAGTTTATGGAAAGTGGTCAACCAGAAGCTAAACAGGAA
GAAGCAAACGAAGAGGTGAAACAAGAAGAAGAAGGTAAATAAGTATTTTGTATTAT
ATAACAAACAAAGTAAGGAATACAGATTTATACAATAAATTGCCATACTAGTCACG
TGAGATATCTCATCCATTCCCCAACTCCCAAGAAAATAAAAAAGTGAAAAATAAAA
TCAAACCCAAAGATCAACCTCCCCATCATCATCGTCATCAAACCCCCAGCTCAATTC
GCAATGGTTAGCACAAAAACATACACAGAAAGGGCATCAGCACACCCCTCCAAGGT
TGCCCAACGTTTATTAATTAAAGGCTAGGTGGAGGCTCAGTGATGATAAGTCTGCGA
TGGTGGATGCATGTGTCATGGTCATAGCTGTTTCCTGTGTGAAATTGTTATCCGCTCA
GAGGGCACAATCCTATTCCGCGCTATCCGACAATCTCCAAGACATTAGGTGGAGTTC
AGTTCGGCGTATGGCATATGTCGCTGGAAAGAACATGTGAGCAAAAGGCCAGCAAA
AGGCCAGGAACCGTAAAAAGGCCGCGTTGCTGGCGTTTTTCCATAGGCTCCGCCCCC
CTGACGAGCATCACAAAAATCGACGCTCAAGTCAGAGGTGGCGAAACCCGACAGGA
CTATAAAGATACCAGGCGTTTCCCCCTGGAAGCTCCCTCGTGCGCTCTCCTGTTCCG
ACCCTGCCGCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCCAAGCTGG
GCTGTGTGCACGAACCCCCCGTTCAGCCCGACCGCTGCGCCTTATCCGGTAACTATC
GTCTTGAGTCCAACCCGGTAAGACACGACTTATCGCCACTGGCAGCAGCCACTGGTA
ACAGGATTAGCAGAGCGAGGTATGTAGGCGGTGCTACAGAGTTCTTGAAGTGGTGG
CCTAACTACGGCTACACTAGAAGAACAGTATTTGGTATCTGCGCTCTGCTGAAGCCA
GTTACCTTCGGAAAAAGAGTTGGTAGCTCTTGATCCGGCAAACAAACCACCGCTGGT
AGCGGTGGTTTTTTTGTTTGCAAGCAGCAGATTACGCGCAGAAAAAAAGGATCTCAA
GAAGATCCTTTGATCTTTTCTACGGGGTCTGACGCTCTATTCAACAAAGCCGCCGTC
CCGTCAAGTCAGCGTAAATGGGTAGGGGGCTTCAAATCGTCCGCTCTGCCAGTGTTA
CAACCAATTAACAAATTCTGATTAGAAAAACTCATCGAGCATCAAATGAAACTGCA
ATTTATTCATATCAGGATTATCAATACCATATTTTTGAAAAAGCCGTTTCTGTAATGA
AGGAGAAAACTCACCGAGGCAGTTCCATAGGATGGCAAGATCCTGGTATCGGTCTG
CGATTCCGACTCGTCCAACATCAATACAACCTATTAATTTCCCCTCGTCAAAAATAA
GGTTATCAAGTGAGAAATCACCATGAGTGACGACTGAATCCGGTGAGAATGGCAAA
AGCTTATGCATTTCTTTCCAGACTTGTTCAACAGGCCAGCCATTACGCTCGTCATCAA
AATCACTCGCATCAACCAAACCGTTATTCATTCGTGATTGCGCCTGAGCGAGACGAA
ATACGCGATCGCTGTTAAAAGGACAATTACAAACAGGAATCGAATGCAACCGGCGC
AGGAACACTGCCAGCGCATCAACAATATTTTCACCTGAATCAGGATATTCTTCTAAT
ACCTGGAATGCTGTTTTCCCGGGGATCGCAGTGGTGAGTAACCATGCATCATCAGGA
GTACGGATAAAATGCTTGATGGTCGGAAGAGGCATAAATTCCGTCAGCCAGTTTAGT
CTGACCATCTCATCTGTAACATCATTGGCAACGCTACCTTTGCCATGTTTCAGAAAC
AACTCTGGCGCATCGGGCTTCCCATACAATCGATAGATTGTCGCACCTGATTGCCCG
ACATTATCGCGAGCCCATTTATACCCATATAAATCAGCATCCATGTTGGAATTTAAT
CGCGGCCTCGAGCAAGACGTTTCCCGTTGAATATGGCTCATAACACCCCTTGTATTA
CTGTTTATGTAAGCAGACAGTTTTATTGTTCATGATGATATATTTTTATCTTGTGCAA
TGTAACATCAGAGATTTTGAGACACAACGTGGCTTTCCCCCGCCGCTCTAGAACTAG
TGGATCCAAATAAAACGAAAGGCTCAGTCGAAAGACTGGGCCTTTCGTTTTATCTGT
TGTTTGTCGCATTATACGAGACGTCCAGGTTGGGATACCTGAAACAAAACCCATCGT
ACGGCCAAGGAAGTCTCCAATAACTGTGATCCACCACAAGCGCCAGGGTTTTCCCA
GTCACGACGTTGTAAAACGACGGCCAGTCATGCATAATCCGCACGCATCTGGAATA
AGGAAGTGCCATTCCGCCTGACCT
pLD87
(SEQ ID NO: 166)
AGGCTAGGTGGAGGCTCAGTGATGATAAGTCTGCGATGGTGGATGCATGTGTCATG
GTCATAGCTGTTTCCTGTGTGAAATTGTTATCCGCTCAGAGGGCACAATCCTATTCCG
CGCTATCCGACAATCTCCAAGACATTAGGTGGAGTTCAGTTCGGCGTATGGCATATG
TCGCTGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAAAA
TCGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCC
GTTCAGCCCGACCGCTGCGCCTTATCCGGTAACTATCGTCTTGAGTCCAACCCGGTA
AGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAG
AAGAACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTT
CTACGGGGTCTGACGCTCTATTCAACAAAGCCGCCGTCCCGTCAAGTCAGCGTAAAT
GGGTAGGGGGCTTCAAATCGTCCGCTCTGCCAGTGTTACAACCAATTAACAAATTCT
GATTAGAAAAACTCATCGAGCATCAAATGAAACTGCAATTTATTCATATCAGGATTA
TCAATACCATATTTTTGAAAAAGCCGTTTCTGTAATGAAGGAGAAAACTCACCGAGG
CAGTTCCATAGGATGGCAAGATCCTGGTATCGGTCTGCGATTCCGACTCGTCCAACA
TCAATACAACCTATTAATTTCCCCTCGTCAAAAATAAGGTTATCAAGTGAGAAATCA
CCATGAGTGACGACTGAATCCGGTGAGAATGGCAAAAGCTTATGCATTTCTTTCCAG
ACTTGTTCAACAGGCCAGCCATTACGCTCGTCATCAAAATCACTCGCATCAACCAAA
CCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATC
AACAATATTTTCACCTGAATCAGGATATTCTTCTAATACCTGGAATGCTGTTTTCCCG
GGGATCGCAGTGGTGAGTAACCATGCATCATCAGGAGTACGGATAAAATGCTTGAT
GGTCGGAAGAGGCATAAATTCCGTCAGCCAGTTTAGTCTGACCATCTCATCTGTAAC
ATCATTGGCAACGCTACCTTTGCCATGTTTCAGAAACAACTCTGGCGCATCGGGCTT
CCCATACAATCGATAGATTGTCGCACCTGATTGCCCGACATTATCGCGAGCCCATTT
ATACCCATATAAATCAGCATCCATGTTGGAATTTAATCGCGGCCTCGAGCAAGACGT
TTCCCGTTGAATATGGCTCATAACACCCCTTGTATTACTGTTTATGTAAGCAGACAGT
TTTATTGTTCATGATGATATATTTTTATCTTGTGCAATGTAACATCAGAGATTTTGAG
ACACAACGTGGCTTTCCCCCGCCGCTCTAGAACTAGTGGATCCAAATAAAACGAAA
GGCTCAGTCGAAAGACTGGGCCTTTCGTTTTATCTGTTGTTTGTCGCATTATACGAGA
CGTCCAGGTTGGGATACCTGAAACAAAACCCATCGTACGGCCAAGGAAGTCTCCAA
TAACTGTGATCCACCACAAGCGCCAGGGTTTTCCCAGTCACGACGTTGTAAAACGAC
GGCCAGTCATGCATAATCCGCACGCATCTGGAATAAGGAAGTGCCATTCCGCCTGA
CCTTTAATTAAGTCGACTTGATGTTTAGAGTGTCCAGATCCGCAAGATCGGCTCGCA
CTTGTGTTGTGTTGTTTCAAATCAGCCTGTCGTTTTGTGTCGTTTGAGATCATTCTGTC
TCACTCTTAGGCTCGCTTAGAACCGACAACGGAGAATCCGGGCTCGGTTTTTCGGTC
GGCCTTGATCTGGGCCTTGGACTTGTACTGGTCGGCCATCTCCACGTTGACCAGCTC
CTTGACCTTGTAGAGCTGACCGGCGATACCAGGAGACACCTTGTAGTACTTCTGGGA
GCCGACCTTGCCCAGACCGAGGGTCTTGAGCACGTCACGTGTTCTCCACGGCATTCG
CAGGATAGATCGGACCTGTGTGACTTTGTAGAACATGGCGTTTCAGGTGGTTGCGTG
AGTGTGTAAAATCGTGTCTTTCAGAAGTTACAAATTTCACCGCATTTAGAGTTTATG
CAGATGGGCGGTGTGTGGTTGGGAGTTCGATTTCCGTGCGTGCATTTGATCTTGATG
AATTGGATTTGTACATGGGGAATAGCACGTCAAGAACCGCCTACTGCAAACTCGTG
AATATTGAGATTATTGAGGAAATTCAAGGAAAATTCAGATCAGATTTGAGAGCAAA
GTCCAACAATACTACACAATCCCTTTCCTGTATTCTTCCACCATCGTCATCGTCGTCT
GTCTTCTCTTCAGCTTTTTAATTTCACTCCCCACAAACCCAAATTTAGCTGCATCATT
CATCAACCTCCAATTATAACTATACATCGCGACACGAACACGAAACACGAACCACG
AACCGCCGCTTTGTCGACGGAGTGAGACGGGAGAGAGACCATGGTCTCTCGCTCGT
CTCACGCTTAGCGGCCGCGTCGACATAACTTCGTATAGCATACATTATACGAAGTTA
TTTTCTAATTTGGACCGATAGCCGTATAGTCCAGTCTATCTATAAGTTCAACTAACTC
GTAACTATTACCATAACATATACTTCACTGCCCCAGATAAGGTTCCGATAAAAAGTT
GTGCAGACTAAATTTATTTCAGTCTCCTCTTCACCACCAAAATGCCCTCCTACGAAG
CGCGAGCTAACGTCCACAAGTCCGCCTTTGCCGCCCGAGTGCTCAAGCTCGTGGCAG
CCAAGAAAACCAACCTGTGTGCTTCTCTGGATGTTACCACCACCAAGGAGCTCATTG
AGCTTGCCGATAAGGTCGGACCTTATGTGTGCATGATCAAGACCCATATCGACATCA
TTGACGACTTCACCTACGCCGGAACTGTGCTCCCCCTCAAGGAACTTGCTCTTAAGC
ACGGTTTCTTCCTGTTCGAGGACAGAAAGTTCGCAGATATTGGCAACACTGTCAAGC
ACCAGTACAAGAACGGTGTCTACCGAATCGCCGAGTGGTCCGATATCACCAACGCC
CACGGTGTACCCGGAACCGGAATCATTGCTGGCCTGCGAGCTGGTGCCGAGGAAAC
TGTCTCTGAACAGAAGAAGGAGGATGTCTCTGACTACGAGAACTCCCAGTACAAGG
AGTTCCTGGTCCCCTCTCCCAACGAGAAGCTGGCCAGAGGTCTGCTCATGCTGGCCG
AGCTGTCTTGCAAGGGCTCTCTGGCCACTGGCGAGTACTCCAAGCAGACCATTGAGC
TTGCCCGATCCGACCCCGAGTTTGTGGTTGGCTTCATTGCCCAGAACCGACCTAAGG
GCGACTCTGAGGACTGGCTTATTCTGACCCCCGGGGTGGGTCTTGACGACAAGGGA
GATGCTCTCGGACAGCAGTACCGAACTGTTGAGGATGTCATGTCTACCGGAACGGAT
ATCATAATTGTCGGCCGAGGTCTGTACGGCCAGAACCGAGATCCTATTGAGGAGGC
CAAGCGATACCAGAAGGCTGGCTGGGAGGCTTACCAGAAGATTAACTGTTAGAGGT
TAGACTATGGATATGTCATTTAACTGTGTATATAGAGAGCGTGCAAGTATGGAGCGC
TTGTTCAGCTTGTATGATGGTCAGACGACCTGTCTGATCGAGTATGTATGATACTGC
ACAACCTGATAACTTCGTATAGCATACATTATACGAAGTTATCTCGAGGGATCCCTA
GGGAGGCACATCTAAACGAATAACGAATATTAATGATACCATCATATCTCAGAACA
TGTATGACTGCTGCTTCCAAACGATATGAGGATGAGTCCTCTTTCAGATTAAGATAG
AGTACAAATATATTATCTATATACTGGTGTCTGTGCGATGTCGTATGAGCGGTGAAT
CATGTGACTGTCACGTGGTTTGGCCCAAGTTACACCGTAGCTACGCCTTTCTTGACC
GACTCCATGGTCTTCTGGGCGGGTTGACAGTTTCCACTGGATGAGCGTCCGCCTCCT
GTTCCTGTCGTTGTCCCTGCAGCTCAGCCTCAATCTTCTGACCGAGCTCGGAGTCCA
GGGAAATGCCAACAGGTTGTCCAAGCAACATCATGGTTTGGTGGGCAGCCGTGATC
TCATCGTCGTTGGATACCATTCGGTACTTGGCCTCAATCTGCACAAAGTAGCGGTAC
CACTGGTTTCGAGCAAACCGCTCCAATTGAGCCTCTCCGTCGAGAGAGAGAGTAGGT
GATTGCTCCAACTTGCGGCCAAAATGAAGTTCTCGACTCACCTTTTTGAAGCGGTTC
TTCTTGCCCATCTTGGTGGCGAAAGTAGTGGCTAGTGGTGGATGACTTTGTATAATG
TACCGATGAAGAGGGTTGTATTTGCTCAGTAAGAAGTAGCGAGTGAAATCAGATCA
CTTAACGAGAGCAAAGGGCAATGGAATACCTGCTGCCTGATTAACAACAGCTTCTGT
GTCGTTTCTCTCTTGTGAATGAGTGTGTTGCTAGAGGTAGGTTGGCACTCCAATGTTA
CTTAATTAA
pLD101
(SEQ ID NO: 167)
AGGCTAGGTGGAGGCTCAGTGATGATAAGTCTGCGATGGTGGATGCATGTGTCATG
GTCATAGCTGTTTCCTGTGTGAAATTGTTATCCGCTCAGAGGGCACAATCCTATTCCG
CGCTATCCGACAATCTCCAAGACATTAGGTGGAGTTCAGTTCGGCGTATGGCATATG
TCGCTGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAAAA
TCGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCC
GTTCAGCCCGACCGCTGCGCCTTATCCGGTAACTATCGTCTTGAGTCCAACCCGGTA
AGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAG
AAGAACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTT
CTACGGGGTCTGACGCTCTATTCAACAAAGCCGCCGTCCCGTCAAGTCAGCGTAAAT
GGGTAGGGGGCTTCAAATCGTCCTCGTGATACCAATTCGGAGCCTGCTTTTTTGTAC
AAACTTGTTGATAATGGCAATTCAAGGATCTTCACCTAGATCCTTTTAAATTAAAAA
TGAAGTTTTAAATCAATCTAAAGTATATATGAGTAAACTTGGTCTGACAGTTACCAA
TGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCA
GTGCTGCAATGATACCGCGAGAGCCACGCTCACCGGCTCCAGATTTATCAGCAATA
AACCAGCCAGCCGGAAGGGCCGAGCGCAGAAGTGGTCCTGCAACTTTATCCGCCTC
CATCCAGTCTATTAATTGTTGCCGGGAAGCTAGAGTAAGTAGTTCGCCAGTTAATAG
TTTGCGCAACGTTGTTGCCATTGCTACAGGCATCGTGGTGTCACGCTCGTCGTTTGGT
ATGGCTTCATTCAGCTCCGGTTCCCAACGATCAAGGCGAGTTACATGATCCCCCATG
TTGTGCAAAAAAGCGGTTAGCTCCTTCGGTCCTCCGATCGTTGTCAGAAGTAAGTTG
GCCGCAGTGTTATCACTCATGGTTATGGCAGCACTGCATAATTCTCTTACTGTCATGC
CATCCGTAAGATGCTTTTCTGTGACTGGTGAGTACTCAACCAAGTCATTCTGAGAAT
AGTGTATGCGGCGACCGAGTTGCTCTTGCCCGGCGTCAATACGGGATAATACCGCGC
CACATAGCAGAACTTTAAAAGTGCTCATCATTGGAAAACGTTCTTCGGGGCGAAAA
CTCTCAAGGATCTTACCGCTGTTGAGATCCAGTTCGATGTAACCCACTCGTGCACCC
AACTGATCTTCAGCATCTTTTACTTTCACCAGCGTTTCTGGGTGAGCAAAAACAGGA
AGGCAAAATGCCGCAAAAAAGGGAATAAGGGCGACACGGAAATGTTGAATACTCA
TACTCTTCCTTTTTCAATATTATTGAAGCATTTATCAGGGTTATTGTCTCATGAGCGG
ATACATATTTGAATGTATTTAGAAAAATAAACAAATAGGGGTTCCGCGCACATTTCC
CCGAAAAGTGCCAGATACCTGAAACAAAACCCATCGTACGGCCAAGGAAGTCTCCA
ATAACTGTGATCCACCACAAGCGCCAGGGTTTTCCCAGTCACGACGTTGTAAAACGA
CGGCCAGTCATGCATAATCCGCACGCATCTGGAATAAGGAAGTGCCATTCCGCCTG
ACCTTTAATTAACGATCCGCGTAAATTCAGAGCAATGCCAAGATCCTTATTTCCGAT
AACAGCGAGATACACATCAGAAGAGGTGGAGACTCTGGCCCCGCACGACCACCACG
TCGTCACTCAAAGCAAAGTCACGATGTAGAAATCACAATCGTCCCCATAAGCACGT
GGATTCCCCTGGTTTTGTTTCGGCCTCGGCAGTGGCAATTCTGGGGTATATAAACCA
GCAAGGTTATGACCTGATTGACCTGCTTGTGGCCTGATAAACCGCCTGACTTTTGTG
ATAAGGTTGGGAGCATGCGGCTCGGGCCATTGTGAGCATTTTCGTCAAGGACTGGCC
AAGTCCAACTGGAAGAGACATGGGCAAAATGTCGACTTTACAGATGTACCCGAAAT
TGCTTCAATTGCTCTCAGCTGCCGGAACCGGATCTATCGGTGCAAGTATCGTACAGT
AGACATGTGCTATTGGTAACCCTCGGTATTGGCTAGGTTTCGTATCAGGGATACAGT
TCAACGCTGATCGCATATGGCATGATTCCGGCTCGACACAGCGACCAAGAACCAAG
CGTGTATGTCGTAGACTTGCAAATCATGTGGGGCTTATCCCCGGATTTCCCCAAGTC
ACGTTTTCACAAAGGCTGTCTCCCGAATGCATGAGCCGAGGCAGGCTAAACTGGTTT
GTTCATGTACCCCACACAACGTAAAGATGCACCCCATGTGCAGTGAAATACCACAA
GTATATATATACCGACCTACCCGAGATAGCAAATTGATTCTACACTTACACTACCAA
TTCTTACATCAAACCAAACCGCTTGAGACCATCCGGTCTCTGATCATCCTCGAGATA
ACTTCGTATAATGTATGCTATACGAAGTTATCAGGTTGTGCAGTATCATACATACTC
GATCAGACAGGTCGTCTGACCATCATACAAGCTGAACAAGCGCTCCATACTTGCAC
GCTCTCTATATACACAGTTAAATGACATATCCATAGTCTAACCTCTAACAGTTAATCT
TCTGGTAAGCCTCCCAGCCAGCCTTCTGGTATCGCTTGGCCTCCTCAATAGGATCTC
GGTTCTGGCCGTACAGACCTCGGCCGACAATTATGATATCCGTTCCGGTAGACATGA
CATCCTCAACAGTTCGGTACTGCTGTCCGAGAGCATCTCCCTTGTCGTCAAGACCCA
CCCCGGGGGTCAGAATAAGCCAGTCCTCAGAGTCGCCCTTAGGTCGGTTCTGGGCA
ATGAAGCCAACCACAAACTCGGGGTCGGATCGGGCAAGCTCAATGGTCTGCTTGGA
GTACTCGCCAGTGGCCAGAGAGCCCTTGCAAGACAGCTCGGCCAGCATGAGCAGAC
CTCTGGCCAGCTTCTCGTTGGGAGAGGGGACCAGGAACTCCTTGTACTGGGAGTTCT
CGTAGTCAGAGACATCCTCCTTCTTCTGTTCAGAGACAGTTTCCTCGGCACCAGCTC
GCAGGCCAGCAATGATTCCGGTTCCGGGTACACCGTGGGCGTTGGTGATATCGGACC
ACTCGGCGATTCGGTAGACACCGTTCTTGTACTGGTGCTTGACAGTGTTGCCAATAT
CTGCGAACTTTCTGTCCTCGAACAGGAAGAAACCGTGCTTAAGAGCAAGTTCCTTGA
GGGGGAGCACAGTTCCGGCGTAGGTGAAGTCGTCAATGATGTCGATATGGGTCTTG
ATCATGCACACATAAGGTCCGACCTTATCGGCAAGCTCAATGAGCTCCTTGGTGGTG
GTAACATCCAGAGAAGCACACAGGTTGGTTTTCTTGGCTGCCACGAGCTTGAGCACT
CGGGCGGCAAAGGCGGACTTGTGGACGTTAGCTCGCGCTTCGTAGGAGGGCATTTT
GGTGGTGAAGAGGAGACTGAAATAAATTTAGTCTGCACAACTTTTTATCGGAACCTT
ATCTGGGGCAGTGAAGTATATGTTATGGTAATAGTTACGAGTTAGTTGAACTTATAG
ATAGACTGGACTATACGGCTATCGGTCCAAATTAGAAAATAACTTCGTATAATGTAT
GCTATACGAAGTTATGTCGACGCGGCCGCACAAGCACTACATGGACGAGGTCAAGG
CTGCCAACAACCCTCGTAACACCCATGCTCCTTACTACGAGACAAAGCTGCGACCCT
TCCTGTTCCGACCCGATGAGGACGAGGAGATTTGCGACCTGGACGAGTAGGTTGTTG
TAATACTATGATTTATTGTGTTTATATGTTATTGATACTATTGAAAGAGTTATTGTGT
AATTTTAGATGCTGTATGTTAACTAGAAGCTCAGATTCTACAAAGAGATCCTCAGAT
CTGAGGAATGATCTACGTTCTGCAATAGAAGGGACAACTGCAGCTCTGAATGACCA
CAAAAAGAATACACCACAAGCAGTTGTAACTGAGCTATTAGCCTTGCTTTCGCACCA
TTCGCTTTTCTGGATGGTAGCCCTTTACTACAAGTAGCTAATATGGAATGTACATTAC
CGTCTCATTACAATATGTATATGCAAGTTCATGGCACTTCATGCACACCAGCCCCTTC
GTTAAGTACCTTCCAAAAAGTGATCAATGATAAGTGATATATCTAATTTAGAGATCT
GGACACACGAACAAGTCGGGAACACAAATCCCGAGATGATTGCCTGCTCAGAGGAG
TCCAATTAGTCTTTACACCATTCGTGCTAATGAAGGGCACAGAATATTCCACTTTGA
AAGTTTAAGATTAAGCTCGGCTCGCAATTATGCATGAAAAATATGTAGGGAGAGAA
CGATCCCACGAGTTCTGTTTGGTTGCGAGAGTGTTCGGGTTTTCTCCTAAAAAGAAT
AGGGGAGGGAAAAATTATCGCCTAAGTCACCATTAATTAA
pLD102
(SEQ ID NO: 168)
AGGCTAGGTGGAGGCTCAGTGATGATAAGTCTGCGATGGTGGATGCATGTGTCATG
GTCATAGCTGTTTCCTGTGTGAAATTGTTATCCGCTCAGAGGGCACAATCCTATTCCG
CGCTATCCGACAATCTCCAAGACATTAGGTGGAGTTCAGTTCGGCGTATGGCATATG
TCGCTGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAAAA
TCGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCC
GTTCAGCCCGACCGCTGCGCCTTATCCGGTAACTATCGTCTTGAGTCCAACCCGGTA
AGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAG
AAGAACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTT
CTACGGGGTCTGACGCTCTATTCAACAAAGCCGCCGTCCCGTCAAGTCAGCGTAAAT
GGGTAGGGGGCTTCAAATCGTCCTCGTGATACCAATTCGGAGCCTGCTTTTTTGTAC
AAACTTGTTGATAATGGCAATTCAAGGATCTTCACCTAGATCCTTTTAAATTAAAAA
TGAAGTTTTAAATCAATCTAAAGTATATATGAGTAAACTTGGTCTGACAGTTACCAA
TGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCA
GTGCTGCAATGATACCGCGAGAGCCACGCTCACCGGCTCCAGATTTATCAGCAATA
AACCAGCCAGCCGGAAGGGCCGAGCGCAGAAGTGGTCCTGCAACTTTATCCGCCTC
CATCCAGTCTATTAATTGTTGCCGGGAAGCTAGAGTAAGTAGTTCGCCAGTTAATAG
TTTGCGCAACGTTGTTGCCATTGCTACAGGCATCGTGGTGTCACGCTCGTCGTTTGGT
ATGGCTTCATTCAGCTCCGGTTCCCAACGATCAAGGCGAGTTACATGATCCCCCATG
TTGTGCAAAAAAGCGGTTAGCTCCTTCGGTCCTCCGATCGTTGTCAGAAGTAAGTTG
GCCGCAGTGTTATCACTCATGGTTATGGCAGCACTGCATAATTCTCTTACTGTCATGC
CATCCGTAAGATGCTTTTCTGTGACTGGTGAGTACTCAACCAAGTCATTCTGAGAAT
AGTGTATGCGGCGACCGAGTTGCTCTTGCCCGGCGTCAATACGGGATAATACCGCGC
CACATAGCAGAACTTTAAAAGTGCTCATCATTGGAAAACGTTCTTCGGGGCGAAAA
CTCTCAAGGATCTTACCGCTGTTGAGATCCAGTTCGATGTAACCCACTCGTGCACCC
AACTGATCTTCAGCATCTTTTACTTTCACCAGCGTTTCTGGGTGAGCAAAAACAGGA
AGGCAAAATGCCGCAAAAAAGGGAATAAGGGCGACACGGAAATGTTGAATACTCA
TACTCTTCCTTTTTCAATATTATTGAAGCATTTATCAGGGTTATTGTCTCATGAGCGG
ATACATATTTGAATGTATTTAGAAAAATAAACAAATAGGGGTTCCGCGCACATTTCC
CCGAAAAGTGCCAGATACCTGAAACAAAACCCATCGTACGGCCAAGGAAGTCTCCA
ATAACTGTGATCCACCACAAGCGCCAGGGTTTTCCCAGTCACGACGTTGTAAAACGA
CGGCCAGTCATGCATAATCCGCACGCATCTGGAATAAGGAAGTGCCATTCCGCCTG
ACCTTTAATTAATTGGCAAATTTTACTGTGGCCTTCAGAACGGTAAAAATAGACCAA
TCAGAATTCTGAAAAGCACATCTTGATCTCCTCATTGCGGGGAGTCCAACGGTGGTC
TTATTCCCCCGAATTTCCCGCTCAATCTCGTTCCAGACCGACCCGGACACAGTGCTT
AACGCCGTTCCGAAACTCTACCGCAGATATGCTCCAACGGACTGGGCTGCATAGAT
GTGATCCTCGGCTTGGAGAAATGGATAAAAGCCGGCCAAAAAAAAAGCGGAAAAA
AGCGGAAAAAAAGAGAAAAAAAATCGCAAAATTTGAAAAATAGGGGGAAAAGACG
CAAAAACGCAAGGAGGGGGGAGTATATGACACTGATAAGCAAGCTCACAACGGTTC
CTCTTATTTTTTTCCTCATCTTCTGCCTAGGTTCCCAAAATCCCAGATGCTTCTCTCCA
GTGCCAAAAGTAAGTACCCCACAGGTTTTCGGCCGAAAATTCCACGTGCAGCAACG
TCGTGTGGGGTGTTAAAATGTGGGGGCGGGGAACCAGGACAAGAGGCTCTTGTGGG
AGCCGAATGAGAGCACAAAGCGGGCGGGTGTGATAAGGGCATTTTTGCCCATTTTC
CCTTCTCCTGTCTCTCCGACGGTGATGGCGTTGTGCGTCCTCTATCTATTTCTTTTTAT
TTCTTTTTGTTTTATTTCTCTGACTACCGATTTGGCTTGATTTCCTCAACCCCACACAA
ATAAGCTCGGGCCGAGGAATATATATATACACGGACACAGTCGCCCTGTGGACAAC
ACGTCACTACCTCTACGACGCTTGAGACCATCCGGTCTCTGATCATCCTCGAGATAA
CTTCGTATAATGTATGCTATACGAAGTTATCAGGTTGTGCAGTATCATACATACTCG
ATCAGACAGGTCGTCTGACCATCATACAAGCTGAACAAGCGCTCCATACTTGCACGC
TCTCTATATACACAGTTAAATGACATATCCATAGTCTAACCTCTAACAGTTAATCTTC
TGGTAAGCCTCCCAGCCAGCCTTCTGGTATCGCTTGGCCTCCTCAATAGGATCTCGG
TTCTGGCCGTACAGACCTCGGCCGACAATTATGATATCCGTTCCGGTAGACATGACA
TCCTCAACAGTTCGGTACTGCTGTCCGAGAGCATCTCCCTTGTCGTCAAGACCCACC
CCGGGGGTCAGAATAAGCCAGTCCTCAGAGTCGCCCTTAGGTCGGTTCTGGGCAATG
AAGCCAACCACAAACTCGGGGTCGGATCGGGCAAGCTCAATGGTCTGCTTGGAGTA
CTCGCCAGTGGCCAGAGAGCCCTTGCAAGACAGCTCGGCCAGCATGAGCAGACCTC
TGGCCAGCTTCTCGTTGGGAGAGGGGACCAGGAACTCCTTGTACTGGGAGTTCTCGT
AGTCAGAGACATCCTCCTTCTTCTGTTCAGAGACAGTTTCCTCGGCACCAGCTCGCA
GGCCAGCAATGATTCCGGTTCCGGGTACACCGTGGGCGTTGGTGATATCGGACCACT
CGGCGATTCGGTAGACACCGTTCTTGTACTGGTGCTTGACAGTGTTGCCAATATCTG
CGAACTTTCTGTCCTCGAACAGGAAGAAACCGTGCTTAAGAGCAAGTTCCTTGAGGG
GGAGCACAGTTCCGGCGTAGGTGAAGTCGTCAATGATGTCGATATGGGTCTTGATCA
TGCACACATAAGGTCCGACCTTATCGGCAAGCTCAATGAGCTCCTTGGTGGTGGTAA
CATCCAGAGAAGCACACAGGTTGGTTTTCTTGGCTGCCACGAGCTTGAGCACTCGGG
CGGCAAAGGCGGACTTGTGGACGTTAGCTCGCGCTTCGTAGGAGGGCATTTTGGTGG
TGAAGAGGAGACTGAAATAAATTTAGTCTGCACAACTTTTTATCGGAACCTTATCTG
GGGCAGTGAAGTATATGTTATGGTAATAGTTACGAGTTAGTTGAACTTATAGATAGA
CTGGACTATACGGCTATCGGTCCAAATTAGAAAATAACTTCGTATAATGTATGCTAT
ACGAAGTTATGTCGACGCGGCCGCATGGAGCGTGTGTTCTGAGTCGATGTTTTCTAT
GGAGTTGTGAGTGTTAGTAGACATGATGGGTTTATATATGATGAATGAATAGATGTG
ATTTTGATTTGCACGATGGAATTGAGAACTTTGTAAACGTACATGGGAATGTATGAA
TGTGGGGGTTTTGTGACTGGATAACTGACGGTCAGTGGACGCCGTTGTTCAAATATC
CAAGAGATGCGAGAAACTTTGGGTCAAGTGAACATGTCCTCTCTGTTCAAGTAAACC
ATCAACTATGGGTAGTATATTTAGTAAGGACAAGAGTTGAGATTCTTTGGAGTCCTA
GAAACGTATTTTCGCGTTCCAAGATCAAATTAGTAGAGTAATACGGGCACGGGAAT
CCATTCATAGTCTCAATTTTCCCATAGGTGTGCTACAAGGTGTTGAGATGTGGTACA
GTACCACCATGATTCGAGGTAAAGAGCCCAGAAGTCATTGATGAGGTCAAGAAATA
CACAGATCTACAGCTCAATACAATGAATATCTTCTTTCATATTCTTCAGGTGACACC
AAGGGTGTCTATTTTCCCCAGAAATGCGTGAAAAGGCGCGTGTGTAGCGTGGAGTAT
GGGTTCGGTTGGCGTATCCTTCATATATCGACGAAATAGTAGGGCAAGAGATGACA
AAAAGTATCTATATGTAGACAGCGTAGAATATGGATTTGATTGGTATAAATTCATTT
ATTGCGTGTCTCACAAATACTCTCGATAAGTTGGGGTTAAACTGGAGATGGAACAAT
GTCGATATCTCGACATATTTTGATATTTGTTAATTAA
pLD111
(SEQ ID NO: 169)
TTAATTAATTCCGCTTAATGGAGTCCAAAAAGACCAACCTCTGCGCCTCGATCGACG
TGACCACAACCGCCGAGTTCCTTTCGCTCATCGACAAGCTCGGTCCCCACATCTGTC
TCGTGAAGACGCACATCGATATCATCTCAGACTTCAGCTACGAGGGCACGATTGAGC
CGTTGCTTGTGCTTGCAGAGCGCCACGGGTTCTTGATATTCGAGGACAGGAAGTTTG
CTGATATCGGAAACACCGTGATGTTGCAGTACACCTCGGGGGTATACCGGATCGCG
GCGTGGAGTGACATCACGAACGCGCACGGAGTGACTGGGAAGGGCGTCGTTGAAGG
GTTGAAACGCGGTGCGGAGGGGGTAGAAAAGGAAAGGGGCGTGTTGATGTTGGCG
GAGTTGTCGAGTAAAGGCTCGTTGGCGCATGGTGAATATACCCGTGAGACGATCGA
GATTGCGAAGAGTGATCGGGAGTTCGTGATTGGGTTCATCGCGCAGCGGGACATGG
GGGGTAGAGAAGAAGGGTTTGATTGGATCATCATGACGCCTGGTGTGGGGTTGGAT
GATAAAGGCGATGCGTTGGGCCAGCAGTATAGGACTGTTGATGAGGTGGTTCTGAC
TGGTACCGATGTGATTATTGTCGGGAGAGGGTTGTTTGGAAAAGGAAGAGACCCTG
AGGTGGAGGGAAAGAGATACAGGGATGCTGGATGGAAGGCATACTTGAAGAGAAC
TGGTCAGTTAGAATAAATATTGTAATAAATAGGTCTATATACATACACTAAGCTTCT
AGGACGTCATTGTAGTCTTCGAAGTTGTCTGCTAGTTTAGTTCTCATGATTTCGAAAA
CCAATAACGCAATGGATGTAGCAGGGATGGTGGTTAGTGCGTTCCTGACAAACCCA
GAGTACGCCGCCTCAAACCACGTCACATTCGCCCTTTGCTTCATCCGCATCACTTGCT
TGAAGGTATCCACGTACGAGTTGTAATACACCTTGAAGAACGGCTTCGTCTAGTTCG
GCATGGCAGATCATCATGCCTGCAGGAGCTCCAATTGTAATATTTCGGGAGAAATAT
CGTTGGGGTAAAACAACAGAGAGAGAGAGGGAGAGATGGTTCTGGTAGAATTATAA
TCTGGTTGTTGCAAATGCTACTGATCGACTCTGGCAATGTCTGTAGCTCGCTAGTTGT
ATGCAACTTAGGTGTTATGCATACACACGGTTATTCGGTTGAATTGTGGAGTAAAAA
TTGTCTGAGTTGTGTCTTAGCTACTGGCTGGCCCCCCGCGAAAGATAATCAAAATTA
CACTTGTGAATTTTTGCACACACACCGATTAACATTTCCCTTTTTTGTCCACCGATAC
ACGCTTGCCTCTTCTTATTTTCTCTGTGCTTCCCCCTCCTGTGACTTTTTCCACCATTG
ATATAAAATCAACTCCATTTCCCTAAAATCTCCCCAGATTCTAAAAACAACTTCTTCT
CTTCTGCTTTTCCTTATTTTTGTTATATTTATTTACCATCCCTTATTTTGAATAGTTATT
CCCCACTAACATTGTTCAAATCTTCACGACataATGAATTGTTCAGCATTTAGTTTTTG
GTTTGTTTGTAAGATTATTTTCTTCTTTTTGTCATTTAACATTCAAATTTCAATTGCAA
ACCCACAAGAAAACTTTTTGAAGTGTTTTTCAGAATACATTCCAAACAATCCAGCTA
ACCCAAAGTTTATTTACACACAACATGATCAATTGTACATGTCAGTTTTGAACTCAA
CAATTCAAAACTTGAGATTTACATCAGATACCACACCAAAGCCATTGGTTATTGTTA
CACCATCAAACGTTTCCCATATTCAAGCATCAATCTTGTGTTCAAAGAAGGTTGGAT
TGCAAATTAGAACCAGATCAGGAGGACACGATGCAGAAGGAATGTCATACATTTCA
CAAGTTCCATTCGTTGTTGTTGATTTGAGAAACATGCACTCAATTAAGATTGATGTTC
ATTCACAAACAGCATGGGTTGAAGCAGGAGCAACATTGGGTGAAGTTTACTACTGG
ATTAACGAAAAGAACGAAAACTTCAGTTTTCCAGGAGGTTACTGTCCAACAGTTGG
AGTTGGAGGACATTTTTCAGGTGGAGGATACGGAGCATTGATGAGAAACTACGGAT
TGGCAGCAGATAACATTATTGATGCACACTTGGTTAACGTTGATGGAAAGGTTTTGG
ATAGAAAGTCAATGGGAGAAGATTTGTTTTGGGCAATTAGAGGAGGTGGTGGAGAG
AACTTTGGAATTATTGCAGCATGGAAGATCAAGTTGGTTGCAGTTCCATCAAAGTCA
ACAATCTTTTCAGTTAAGAAGAACATGGAAATTCATGGTTTGGTTAAGTTGTTTAAC
AAGTGGCAAAACATTGCATACAAGTACGATAAGGATTTGGTTTTGATGACACATTTT
ATTACAAAGAACATTACAGATAACCATGGAAAGAACAAGACAACAGTTCACGGATA
CTTTTCATCAATTTTTCACGGAGGAGTTGATTCATTGGTTGACTTGATGAACAAGTCA
TTTCCAGAATTGGGAATCAAGAAGACAGATTGTAAGGAATTTTCATGGATTGATACA
ACAATTTTCTACTCAGGAGTTGTTAACTTTAACACAGCAAACTTTAAGAAGGAAATT
TTGTTGGACAGATCAGCAGGAAAGAAGACCGCATTTTCCATTAAGTTGGATTACGTT
AAGAAACCAATTCCAGAAACAGCAATGGTTAAGATTTTGGAAAAGTTGTACGAAGA
AGATGTTGGTGTTGGAATGTACGTTTTGTACCCATACGGAGGAATTATGGAAGAAAT
CTCAGAATCAGCAATTCCATTTCCACATAGAGCAGGTATTATGTACGAATTGTGGTA
CACAGCATCATGGGAAAAGCAAGAAGATAATGAAAAGCATATTAACTGGGTTAGAT
CAGTTTACAACTTTACAACACCATACGTTTCACAAAACCCAAGATTGGCATACTTGA
ACTACAGAGATTTGGATTTGGGAAAGACAAACCCAGAATCACCAAACAACTATACA
CAAGCTAGAATTTGGGGAGAAAAGTACTTTGGTAAGAACTTCAACAGATTGGTTAA
AGTTAAGACAAAGGCAGATCCAAATAACTTCTTTAGAAACGAACAATCAATTCCAC
CATTGCCACCACATCATCATTAATAAGAGTGACTCTTTTGATAAGAGTCGCAAATTT
GATTTCATAAGTATATATTCATTATGTAAAGTAGTAAATGGAAAATTCATTAAAAAA
AAAGCAAATTTCCGTTGTATGCATACTCCGAACACAAAACTAGCCCCGGAAAAACC
CTTAGTTGATAGTTGCGAATTTAGGTCGACCATATGCGACGGGTACAACGAGAATTG
TATTGAATTGATCAAGAACATGATCTTGGTGTTACAGAACATCAAGTTCTTGGACCA
GACTGAGAATGCACAGATATACAAGGCGTCATGTGATAAAATGGATGAGATTTATC
CACAATTGAAGAAAGAGTTTATGGAAAGTGGTCAACCAGAAGCTAAACAGGAAGA
AGCAAACGAAGAGGTGAAACAAGAAGAAGAAGGTAAATAAGTATTTTGTATTATAT
AACAAACAAAGTAAGGAATACAGATTTATACAATAAATTGCCATACTAGTCACGTG
AGATATCTCATCCATTCCCCAACTCCCAAGAAAATAAAAAAGTGAAAAATAAAATC
AAACCCAAAGATCAACCTCCCCATCATCATCGTCATCAAACCCCCAGCTCAATTCGC
AATGGTTAGCACAAAAACATACACAGAAAGGGCATCAGCACACCCCTCCAAGGTTG
CCCAACGTTTATTAATTAAAGGCTAGGTGGAGGCTCAGTGATGATAAGTCTGCGATG
GTGGATGCATGTGTCATGGTCATAGCTGTTTCCTGTGTGAAATTGTTATCCGCTCAGA
GGGCACAATCCTATTCCGCGCTATCCGACAATCTCCAAGACATTAGGTGGAGTTCAG
TTCGGCGTATGGCATATGTCGCTGGAAAGAACATGTGAGCAAAAGGCCAGCAAAAG
GCCAGGAACCGTAAAAAGGCCGCGTTGCTGGCGTTTTTCCATAGGCTCCGCCCCCCT
GACGAGCATCACAAAAATCGACGCTCAAGTCAGAGGTGGCGAAACCCGACAGGACT
ATAAAGATACCAGGCGTTTCCCCCTGGAAGCTCCCTCGTGCGCTCTCCTGTTCCGAC
CCTGCCGCTTACCGGATACCTGTCCGCCTTTCTCCCTTCGGGAAGCGTGGCGCTTTCT
CATAGCTCACGCTGTAGGTATCTCAGTTCGGTGTAGGTCGTTCGCTCCAAGCTGGGC
TGTGTGCACGAACCCCCCGTTCAGCCCGACCGCTGCGCCTTATCCGGTAACTATCGT
CTTGAGTCCAACCCGGTAAGACACGACTTATCGCCACTGGCAGCAGCCACTGGTAA
CAGGATTAGCAGAGCGAGGTATGTAGGCGGTGCTACAGAGTTCTTGAAGTGGTGGC
CTAACTACGGCTACACTAGAAGAACAGTATTTGGTATCTGCGCTCTGCTGAAGCCAG
TTACCTTCGGAAAAAGAGTTGGTAGCTCTTGATCCGGCAAACAAACCACCGCTGGTA
GCGGTGGTTTTTTTGTTTGCAAGCAGCAGATTACGCGCAGAAAAAAAGGATCTCAAG
AAGATCCTTTGATCTTTTCTACGGGGTCTGACGCTCTATTCAACAAAGCCGCCGTCCC
GTCAAGTCAGCGTAAATGGGTAGGGGGCTTCAAATCGTCCGCTCTGCCAGTGTTACA
ACCAATTAACAAATTCTGATTAGAAAAACTCATCGAGCATCAAATGAAACTGCAATT
TATTCATATCAGGATTATCAATACCATATTTTTGAAAAAGCCGTTTCTGTAATGAAG
GAGAAAACTCACCGAGGCAGTTCCATAGGATGGCAAGATCCTGGTATCGGTCTGCG
ATTCCGACTCGTCCAACATCAATACAACCTATTAATTTCCCCTCGTCAAAAATAAGG
TTATCAAGTGAGAAATCACCATGAGTGACGACTGAATCCGGTGAGAATGGCAAAAG
CTTATGCATTTCTTTCCAGACTTGTTCAACAGGCCAGCCATTACGCTCGTCATCAAAA
TCACTCGCATCAACCAAACCGTTATTCATTCGTGATTGCGCCTGAGCGAGACGAAAT
ACGCGATCGCTGTTAAAAGGACAATTACAAACAGGAATCGAATGCAACCGGCGCAG
GAACACTGCCAGCGCATCAACAATATTTTCACCTGAATCAGGATATTCTTCTAATAC
CTGGAATGCTGTTTTCCCGGGGATCGCAGTGGTGAGTAACCATGCATCATCAGGAGT
ACGGATAAAATGCTTGATGGTCGGAAGAGGCATAAATTCCGTCAGCCAGTTTAGTCT
GACCATCTCATCTGTAACATCATTGGCAACGCTACCTTTGCCATGTTTCAGAAACAA
CTCTGGCGCATCGGGCTTCCCATACAATCGATAGATTGTCGCACCTGATTGCCCGAC
ATTATCGCGAGCCCATTTATACCCATATAAATCAGCATCCATGTTGGAATTTAATCG
CGGCCTCGAGCAAGACGTTTCCCGTTGAATATGGCTCATAACACCCCTTGTATTACT
GTTTATGTAAGCAGACAGTTTTATTGTTCATGATGATATATTTTTATCTTGTGCAATG
TAACATCAGAGATTTTGAGACACAACGTGGCTTTCCCCCGCCGCTCTAGAACTAGTG
GATCCAAATAAAACGAAAGGCTCAGTCGAAAGACTGGGCCTTTCGTTTTATCTGTTG
TTTGTCGCATTATACGAGACGTCCAGGTTGGGATACCTGAAACAAAACCCATCGTAC
GGCCAAGGAAGTCTCCAATAACTGTGATCCACCACAAGCGCCAGGGTTTTCCCAGTC
ACGACGTTGTAAAACGACGGCCAGTCATGCATAATCCGCACGCATCTGGAATAAGG
AAGTGCCATTCCGCCTGACCT
pLD112
(SEQ ID NO: 170)
TTAATTAATTCCGCTTAATGGAGTCCAAAAAGACCAACCTCTGCGCCTCGATCGACG
TGACCACAACCGCCGAGTTCCTTTCGCTCATCGACAAGCTCGGTCCCCACATCTGTC
TCGTGAAGACGCACATCGATATCATCTCAGACTTCAGCTACGAGGGCACGATTGAGC
CGTTGCTTGTGCTTGCAGAGCGCCACGGGTTCTTGATATTCGAGGACAGGAAGTTTG
CTGATATCGGAAACACCGTGATGTTGCAGTACACCTCGGGGGTATACCGGATCGCG
GCGTGGAGTGACATCACGAACGCGCACGGAGTGACTGGGAAGGGCGTCGTTGAAGG
GTTGAAACGCGGTGCGGAGGGGGTAGAAAAGGAAAGGGGCGTGTTGATGTTGGCG
GAGTTGTCGAGTAAAGGCTCGTTGGCGCATGGTGAATATACCCGTGAGACGATCGA
GATTGCGAAGAGTGATCGGGAGTTCGTGATTGGGTTCATCGCGCAGCGGGACATGG
GGGGTAGAGAAGAAGGGTTTGATTGGATCATCATGACGCCTGGTGTGGGGTTGGAT
GATAAAGGCGATGCGTTGGGCCAGCAGTATAGGACTGTTGATGAGGTGGTTCTGAC
TGGTACCGATGTGATTATTGTCGGGAGAGGGTTGTTTGGAAAAGGAAGAGACCCTG
AGGTGGAGGGAAAGAGATACAGGGATGCTGGATGGAAGGCATACTTGAAGAGAAC
TGGTCAGTTAGAATAAATATTGTAATAAATAGGTCTATATACATACACTAAGCTTCT
AGGACGTCATTGTAGTCTTCGAAGTTGTCTGCTAGTTTAGTTCTCATGATTTCGAAAA
CCAATAACGCAATGGATGTAGCAGGGATGGTGGTTAGTGCGTTCCTGACAAACCCA
GAGTACGCCGCCTCAAACCACGTCACATTCGCCCTTTGCTTCATCCGCATCACTTGCT
TGAAGGTATCCACGTACGAGTTGTAATACACCTTGAAGAACGGCTTCGTCTAGTTCG
GCATGGCAGATCATCATGCCTGCAGGAGCTCCAATTGTAATATTTCGGGAGAAATAT
CGTTGGGGTAAAACAACAGAGAGAGAGAGGGAGAGATGGTTCTGGTAGAATTATAA
TCTGGTTGTTGCAAATGCTACTGATCGACTCTGGCAATGTCTGTAGCTCGCTAGTTGT
ATGCAACTTAGGTGTTATGCATACACACGGTTATTCGGTTGAATTGTGGAGTAAAAA
TTGTCTGAGTTGTGTCTTAGCTACTGGCTGGCCCCCCGCGAAAGATAATCAAAATTA
CACTTGTGAATTTTTGCACACACACCGATTAACATTTCCCTTTTTTGTCCACCGATAC
ACGCTTGCCTCTTCTTATTTTCTCTGTGCTTCCCCCTCCTGTGACTTTTTCCACCATTG
ATATAAAATCAACTCCATTTCCCTAAAATCTCCCCAGATTCTAAAAACAACTTCTTCT
CTTCTGCTTTTCCTTATTTTTGTTATATTTATTTACCATCCCTTATTTTGAATAGTTATT
CCCCACTAACATTGTTCAAATCTTCACGACataATGAATTGTAGCACTTTCTCATTCTG
GTTTGTTTGTAAGATTATTTTCTTTTTCTTGTCATTTAACATTCAAATTTCAATTGCAA
ACCCACAAGAGAACTTTTTGAAGTGTTTCTCAGAATACATTCCAAACAACCCAGCTA
ACCCAAAGTTTATTTACACCCAACACGATCAATTGTACATGTCAGTTTTGAACTCAA
CAATTCAAAACTTGAGATTTACATCAGATACAACACCAAAGCCATTGGTTATTGTTA
CACCATCAAACGTTAGTCATATTCAAGCATCAATCTTGTGTTCAAAGAAGGTTGGAT
TGCAAATTAGAACTAGATCAGGAGGACATGATGCAGAAGGATTGTCATACATTTCA
CAAGTTCCATTTGCAATTGTTGATTTGAGAAACATGCACACAGTTAAGGTTGATATT
CATTCACAAACAGCATGGGTTGAAGCAGGAGCAACATTGGGTGAAGTTTACTACTG
GATTAACGAAATGAACGAAAACTTCTCATTTCCAGGAGGATACTGTCCAACAGTTGG
TGTTGGAGGACACTTTTCAGGTGGTGGATACGGAGCATTGATGAGAAACTACGGATT
GGCAGCAGATAACATTATTGATGCACATTTGGTTAACGTTGATGGAAAGGTTTTGGA
TAGAAAGTCAATGGGAGAAGATTTGTTTTGGGCAATTAGAGGAGGTGGAGGAGAAA
ACTTTGGAATCATTGCAGCATGTAAGATCAAGTTGGTTGTTGTTCCATCAAAGGCAA
CAATCTTTTCAGTTAAGAAGAACATGGAAATCCATGGATTGGTTAAGTTGTTTAACA
AGTGGCAAAACATTGCATACAAGTACGATAAGGATTTGATGTTGACAACACATTTTA
GAACAAGAAACATTACAGATAACCACGGAAAGAATAAGACAACAGTTCATGGATAC
TTTTCATCAATTTTCTTGGGAGGAGTTGATTCATTGGTTGACTTGATGAACAAGAGTT
TTCCAGAATTGGGAATCAAGAAGACAGATTGTAAGGAATTGTCATGGATCGATACA
ACCATTTTCTACTCAGGAGTTGTTAACTACAACACAGCTAACTTTAAGAAGGAAATT
TTGTTGGACAGATCAGCAGGTAAAAAGACAGCATTTTCAATTAAGTTGGATTACGTT
AAGAAATTGATTCCAGAAACAGCAATGGTTAAGATTTTGGAAAAGTTGTACGAAGA
AGAAGTTGGAGTTGGAATGTACGTTTTGTACCCATACGGAGGAATTATGGATGAAAT
TTCAGAATCAGCAATTCCATTTCCACATAGAGCAGGTATTATGTACGAATTGTGGTA
CACAGCAACATGGGAAAAGCAAGAAGATAACGAAAAGCATATTAACTGGGTTAGAT
CAGTTTACAACTTTACAACCCCATACGTTTCACAAAACCCAAGATTGGCATACTTGA
ACTACAGAGATTTGGATTTGGGAAAGACAAACCCAGAATCACCAAATAACTACACA
CAAGCTAGAATTTGGGGAGAAAAGTACTTTGGTAAGAACTTTAACAGATTGGTGAA
GGTTAAGACAAAGGCAGACCCAAACAATTTCTTTAGAAACGAACAATCAATTCCAC
CATTGCCACCAAGACATCATTAATAAGAGTGACTCTTTTGATAAGAGTCGCAAATTT
GATTTCATAAGTATATATTCATTATGTAAAGTAGTAAATGGAAAATTCATTAAAAAA
AAAGCAAATTTCCGTTGTATGCATACTCCGAACACAAAACTAGCCCCGGAAAAACC
CTTAGTTGATAGTTGCGAATTTAGGTCGACCATATGCGACGGGTACAACGAGAATTG
TATTGAATTGATCAAGAACATGATCTTGGTGTTACAGAACATCAAGTTCTTGGACCA
GACTGAGAATGCACAGATATACAAGGCGTCATGTGATAAAATGGATGAGATTTATC
CACAATTGAAGAAAGAGTTTATGGAAAGTGGTCAACCAGAAGCTAAACAGGAAGA
AGCAAACGAAGAGGTGAAACAAGAAGAAGAAGGTAAATAAGTATTTTGTATTATAT
AACAAACAAAGTAAGGAATACAGATTTATACAATAAATTGCCATACTAGTCACGTG
AGATATCTCATCCATTCCCCAACTCCCAAGAAAATAAAAAAGTGAAAAATAAAATC
AAACCCAAAGATCAACCTCCCCATCATCATCGTCATCAAACCCCCAGCTCAATTCGC
AATGGTTAGCACAAAAACATACACAGAAAGGGCATCAGCACACCCCTCCAAGGTTG
CCCAACGTTTATTAATTAAAGGCTAGGTGGAGGCTCAGTGATGATAAGTCTGCGATG
GTGGATGCATGTGTCATGGTCATAGCTGTTTCCTGTGTGAAATTGTTATCCGCTCAGA
GGGCACAATCCTATTCCGCGCTATCCGACAATCTCCAAGACATTAGGTGGAGTTCAG
TTCGGCGTATGGCATATGTCGCTGGAAAGAACATGTGAGCAAAAGGCCAGCAAAAG
GCCAGGAACCGTAAAAAGGCCGCGTTGCTGGCGTTTTTCCATAGGCTCCGCCCCCCT
GACGAGCATCACAAAAATCGACGCTCAAGTCAGAGGTGGCGAAACCCGACAGGACT
ATAAAGATACCAGGCGTTTCCCCCTGGAAGCTCCCTCGTGCGCTCTCCTGTTCCGAC
CCTGCCGCTTACCGGATACCTGTCCGCCTTTCTCCCTTCGGGAAGCGTGGCGCTTTCT
CATAGCTCACGCTGTAGGTATCTCAGTTCGGTGTAGGTCGTTCGCTCCAAGCTGGGC
TGTGTGCACGAACCCCCCGTTCAGCCCGACCGCTGCGCCTTATCCGGTAACTATCGT
CTTGAGTCCAACCCGGTAAGACACGACTTATCGCCACTGGCAGCAGCCACTGGTAA
CAGGATTAGCAGAGCGAGGTATGTAGGCGGTGCTACAGAGTTCTTGAAGTGGTGGC
CTAACTACGGCTACACTAGAAGAACAGTATTTGGTATCTGCGCTCTGCTGAAGCCAG
TTACCTTCGGAAAAAGAGTTGGTAGCTCTTGATCCGGCAAACAAACCACCGCTGGTA
GCGGTGGTTTTTTTGTTTGCAAGCAGCAGATTACGCGCAGAAAAAAAGGATCTCAAG
AAGATCCTTTGATCTTTTCTACGGGGTCTGACGCTCTATTCAACAAAGCCGCCGTCCC
GTCAAGTCAGCGTAAATGGGTAGGGGGCTTCAAATCGTCCGCTCTGCCAGTGTTACA
ACCAATTAACAAATTCTGATTAGAAAAACTCATCGAGCATCAAATGAAACTGCAATT
TATTCATATCAGGATTATCAATACCATATTTTTGAAAAAGCCGTTTCTGTAATGAAG
GAGAAAACTCACCGAGGCAGTTCCATAGGATGGCAAGATCCTGGTATCGGTCTGCG
ATTCCGACTCGTCCAACATCAATACAACCTATTAATTTCCCCTCGTCAAAAATAAGG
TTATCAAGTGAGAAATCACCATGAGTGACGACTGAATCCGGTGAGAATGGCAAAAG
CTTATGCATTTCTTTCCAGACTTGTTCAACAGGCCAGCCATTACGCTCGTCATCAAAA
TCACTCGCATCAACCAAACCGTTATTCATTCGTGATTGCGCCTGAGCGAGACGAAAT
ACGCGATCGCTGTTAAAAGGACAATTACAAACAGGAATCGAATGCAACCGGCGCAG
GAACACTGCCAGCGCATCAACAATATTTTCACCTGAATCAGGATATTCTTCTAATAC
CTGGAATGCTGTTTTCCCGGGGATCGCAGTGGTGAGTAACCATGCATCATCAGGAGT
ACGGATAAAATGCTTGATGGTCGGAAGAGGCATAAATTCCGTCAGCCAGTTTAGTCT
GACCATCTCATCTGTAACATCATTGGCAACGCTACCTTTGCCATGTTTCAGAAACAA
CTCTGGCGCATCGGGCTTCCCATACAATCGATAGATTGTCGCACCTGATTGCCCGAC
ATTATCGCGAGCCCATTTATACCCATATAAATCAGCATCCATGTTGGAATTTAATCG
CGGCCTCGAGCAAGACGTTTCCCGTTGAATATGGCTCATAACACCCCTTGTATTACT
GTTTATGTAAGCAGACAGTTTTATTGTTCATGATGATATATTTTTATCTTGTGCAATG
TAACATCAGAGATTTTGAGACACAACGTGGCTTTCCCCCGCCGCTCTAGAACTAGTG
GATCCAAATAAAACGAAAGGCTCAGTCGAAAGACTGGGCCTTTCGTTTTATCTGTTG
TTTGTCGCATTATACGAGACGTCCAGGTTGGGATACCTGAAACAAAACCCATCGTAC
GGCCAAGGAAGTCTCCAATAACTGTGATCCACCACAAGCGCCAGGGTTTTCCCAGTC
ACGACGTTGTAAAACGACGGCCAGTCATGCATAATCCGCACGCATCTGGAATAAGG
AAGTGCCATTCCGCCTGACCT
pLD113
(SEQ ID NO: 171)
AGGCTAGGTGGAGGCTCAGTGATGATAAGTCTGCGATGGTGGATGCATGTGTCATG
GTCATAGCTGTTTCCTGTGTGAAATTGTTATCCGCTCAGAGGGCACAATCCTATTCCG
CGCTATCCGACAATCTCCAAGACATTAGGTGGAGTTCAGTTCGGCGTATGGCATATG
TCGCTGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAAAA
TCGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCC
GTTCAGCCCGACCGCTGCGCCTTATCCGGTAACTATCGTCTTGAGTCCAACCCGGTA
AGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAG
AAGAACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTT
CTACGGGGTCTGACGCTCTATTCAACAAAGCCGCCGTCCCGTCAAGTCAGCGTAAAT
GGGTAGGGGGCTTCAAATCGTCCGCTCTGCCAGTGTTACAACCAATTAACAAATTCT
GATTAGAAAAACTCATCGAGCATCAAATGAAACTGCAATTTATTCATATCAGGATTA
TCAATACCATATTTTTGAAAAAGCCGTTTCTGTAATGAAGGAGAAAACTCACCGAGG
CAGTTCCATAGGATGGCAAGATCCTGGTATCGGTCTGCGATTCCGACTCGTCCAACA
TCAATACAACCTATTAATTTCCCCTCGTCAAAAATAAGGTTATCAAGTGAGAAATCA
CCATGAGTGACGACTGAATCCGGTGAGAATGGCAAAAGCTTATGCATTTCTTTCCAG
ACTTGTTCAACAGGCCAGCCATTACGCTCGTCATCAAAATCACTCGCATCAACCAAA
CCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATC
AACAATATTTTCACCTGAATCAGGATATTCTTCTAATACCTGGAATGCTGTTTTCCCG
GGGATCGCAGTGGTGAGTAACCATGCATCATCAGGAGTACGGATAAAATGCTTGAT
GGTCGGAAGAGGCATAAATTCCGTCAGCCAGTTTAGTCTGACCATCTCATCTGTAAC
ATCATTGGCAACGCTACCTTTGCCATGTTTCAGAAACAACTCTGGCGCATCGGGCTT
CCCATACAATCGATAGATTGTCGCACCTGATTGCCCGACATTATCGCGAGCCCATTT
ATACCCATATAAATCAGCATCCATGTTGGAATTTAATCGCGGCCTCGAGCAAGACGT
TTCCCGTTGAATATGGCTCATAACACCCCTTGTATTACTGTTTATGTAAGCAGACAGT
TTTATTGTTCATGATGATATATTTTTATCTTGTGCAATGTAACATCAGAGATTTTGAG
ACACAACGTGGCTTTCCCCCGCCGCTCTAGAACTAGTGGATCCAAATAAAACGAAA
GGCTCAGTCGAAAGACTGGGCCTTTCGTTTTATCTGTTGTTTGTCGCATTATACGAGA
CGTCCAGGTTGGGATACCTGAAACAAAACCCATCGTACGGCCAAGGAAGTCTCCAA
TAACTGTGATCCACCACAAGCGCCAGGGTTTTCCCAGTCACGACGTTGTAAAACGAC
GGCCAGTCATGCATAATCCGCACGCATCTGGAATAAGGAAGTGCCATTCCGCCTGA
CCTGATCCCAATATTACACCCAAGTAGCATGCATAAGCTAAAAGTAACTCGCAGCG
CACACCGTGCAGATTCATAAGTCTATGATTAATTGAACGCCAATAACCCGGCTTACT
ACAAGTACAAGTAGGTATACATAGCGGTAATGAATCATTAGAAAAATAAAAAACAA
AAAAAAACAAAACAAACTGTTGTGGATGCATCAACAGTAGTACATAGTTGTACGAT
GTACTTGTACTTGTAAAAGCAAAAATGTACAATATCTCAGGGAGCGCAACTTTTACG
TTCGAAGAACAATGTACCGCATACCGCATTCTAGATTCTGCGGAACGTCTAACCTGG
AAATACGATTTTTTATTTCTTTCATTTTTTTTGCTTCTTCAAAAGTATGGTAATTTCCT
ACCATTACAGTTGACACTGAACGAGGGGGGATTGAATTTAAGCAAAAAATTAAATC
AAAATACCTTTATGTATCCAGCCCATGTAATAAACAAAAGGATTATATAACAAGAA
ATAAATATATACCTTTAATGGATCATTAGAATAAAAATAAATACGAGAAGCACACC
AGAGAAGCTTTTTGATTGCCACTATACCGCTACTTTGGTATATCTTATTATAATTGTT
GAATTTGCAAGATAGAATGTCATTCATTGGAGAGAAATCCAAGGAATATGTGGGAT
GAAATGACTAGAAGTATGAACAATGAGAATAGTACATACTTGTACCTGTATTTCTAG
AAGAGAGAAAGACAGTTGAGTGTGTGATTCTCGTCCAATAATAATCTCAATAGTAA
CGTGTGAATAGCTGTTCTTTGATAGTTGATATTTCTCGATGACTATTTATGTTGTACA
AGGGATTTTTTTCGTTGCTGTTGATTTCGAATTAGGCAATGCAGATATCATTTATGCT
ATCCATATTTAAGATTTCCCATACGCATTTATAACATTTATTCTACATAAATTGTTAA
ATGAACGAACTGCCATTATAAATTGTTTCCTAAATAGGAAGTGTTTTTCATAAAGCA
AGTAAGTTGTCTAATAATACTAAGTAATAAAAATAAGTTCATACAATATATTTTGAG
AACATCATTTGGAGGCGGTAGATGGAGTCTGTTTATTATTAAACAATGCGAGATGAC
CCCTTAAATATTGAGAACATCAGTTGGAGGCGGCAGATGGAGTCTGTCTATTTAGCA
ATGGGACATGACTGTCAGTATCATCATATGTATATATATAATACATATAATATTATA
TAACACGATTTTTTTAAATTATTGGCCCGAAAATTAATCAGTGTAGACTGGATCCTC
GAGAACCATTTAATTAACAAGTCGAGAACGTACCACTGTCCTCCACTACAAACACA
CCCAATCTGCTTCTTCTAGTCAAGGTTGCTACACCGGTAAATTATAAATCATCATTTC
ATTAGCAGGGCTGGGCCCTTTTTATAGAGTCTTATACACTAGCGGACCCTGCCGGTA
GACCAACCCGCAGGCGCGTCAGTTTGCTCCTTCCATCAATGCGTCGTAGAAACGACT
TACTCCTTCTTGAGCAGCTCCTTGACCTTGTTGGCAACAAAGTCTCCGACCTCGGAG
GTGGAGGAGGAGCCTCCGATATCGGCGGTAGTGATACCAGCCTCGACGGACTCCTT
GACGGCAGCCTCAACAGCGTCACCGGCGGGCTTCATGTTAAGAGAGAACTTGAGCA
TCATGGCGGCAGACAGAATGGTGGCAATGGGGTTGACCTTCTGCTTGCCGAGATCG
GGGGCAGATCCGTGACAGGGCTCGTACAGACCGAACGCCTCGTTGGTGTCGGGCAG
AGAAGCCAGAGAGGCGGAGGGCAGCAGACCCAGAGAACCGGGGATGACGGAGGCC
TCGTCGGAGATGATATCGCCAAACATGTTGGTGGTGATGATGATACCATTCATCTTG
GAGGGCTGCTTGATGAGGATCATGGCGGCCGAGTCGATCAGCTGGTGGTTGAGCTC
CAGCTGGGGGAATTCGTCCTTGAGGACTCGGGTGACGGTCTTTCGCCAAAGTCGAGA
GGAGGCCAGCACGTTGGCCTTGTCAAGGGACCACACGGGAAGAGGGGGGTTGTGCT
GAAGGGCCAGGAAGGCGGCCATTCGGGCAATTCGCTCAACCTCAGGAACGGAGTAA
GTCTCAGTGTCGGAAGCGACGCCAGATCCGTCATCCTCCTTTCGCTCTCCAAAGTAG
ATACCTCCGACGAGCTCTCGGACAATGATGAAGTCGGTGCCCTCAACGTTTCGGATG
GGGGAGAGATCGGCGAGCTTGGGCGACAGCAGCTGGCAGGGTCGCAGGTTGGCGTA
CAGGTTCAGGTCCTTTCGCAGCTTGAGAAGACCCTGCTCGGGTCGCACGTCGGTTCG
TCCGTCGGGAGTGGTCCATACGGTGTTGGCAGCGCCTCCGACAGCACCGAGCATAA
TAGAGTCAGCCTTTCGGCAGATGTCGAGAGTAGCGTCGGTGATGGGCTCGCCCTCCT
TCTCAATGGCAGCTCCTCCAATGAGTCGGTCCTCAAACACAAACTCGGTGCCGGAGG
CCTCAGCAACAGACTTGAGCACCTTGACGGCCTCGGCAATCACCTCGGGGCCACAG
AAGTCGCCGCCGAGAAGAACAATCTTCTTGGAGTCAGTCTTGGTCTTCTTAGTTTCG
GGTTCCATTGTGGATGTGTGTGGTTGTATGTGTGATGTGGTGTGTGGAGTGAAAATC
TGTGGCTGGCAAACGCTCTTGTATATATACGCACTTTTGCCCGTGCTATGTGGAAGA
CTAAACCTCCGAAGATTGTGACTCAGGTAGTGCGGTATCGGCTAGGGACCCAAACCT
TGTCGATGCCGATAGCGCTATCGAACGTACCCCAGCCGGCCGGGAGTATGTCGGAG
GGGACATACGAGATCGTCAAGGGTTTGTGGCCAACTGGTAAATAAATGATGACTCA
GGCGACGACGGAATTCGACAGCAACTACTCCTTTCACCAACCATGTGCATTTTAGCT
CGAATAACATTCACAGGCTTGGTGATCTACATCCATGGTGTCTGGCCGATTACCGTG
GTGTTTTGGCAGTAACGAGAATATTGAGTGAACTCTTCCCATCACCAATAAAGACTC
ATACTACAATCACGAGCGCTTCAGCTGCCACTATAGTGTTGGTGACACAATACCCCT
CGATGCTGGGCATTACTGTAGCAAGAGATATTATTTCATGGCGCATTTTCCAGTCTA
CCTGACTTTTTAGTGTGATTTCTTCTCCACATTTTATGCTCAGTGTGAAAAGTTGGAG
TGCACACTTAATTATCGCCGGTTTTCGGAAAGTACTATGTGCTCAAGGTTGCACCCC
ACGTTACGTATGCAGCACATTGAGCAGCCTTTGGACCGTGGAGATAACGGTGTGGA
GATAGCAACGGGTAGTCTTCGTATTAATTCAATGCATTGTTAGTTTTATATGATATGG
TGTCGAGCGGCCGCGACCGGGTTGGCGGCGCATTTGTGTCCCAAAAAACAGCCCCA
ATTGCCCCAATTGACCCCAAATTGACCCAGTAGCGGGCCCAACCCCGGCGAGAGCC
CCCTTCTCCCCACATATCAAACCTCCCCCGGTTCCCACACTTGCCGTTAAGGGCGTA
GGGTACTGCAGTCTGGAATCTACGCTTGTTCAGACTTTGTACTAGTTTCTTTGTCTGG
CCATCCGGGTAACCCATGCCGGACGCAAAATAGACTACTGAAAATTTTTTTGCTTTG
TGGTTGGGACTTTAGCCAAGGGTATAAAAGACCACCGTCCCCGAATTACCTTTCCTC
TTCTTTTCTCTCTCTCCTTGTCAACTCACACCCGAAATCGTTAAGCATTTCCTTCTGAG
TATAAGAATCATTCAAAATGTCCAACCTCCTCACCGTCCACCAGAACCTTCCCGCCC
TTCCCGTGGACGCCACCTCCGACGAGGTCCGAAAGAACCTCATGGACATGTTCCGA
GATCGACAGGCCTTCTCCGAGCACACTTGGAAGATGCTCCTCTCCGTCTGCCGATCC
TGGGCCGCCTGGTGCAAGCTCAACAACCGAAAGTGGTTCCCCGCCGAGCCCGAGGA
CGTCCGAGACTACCTCCTTTACCTCCAGGCCCGAGGCCTCGCCGTCAAGACCATCCA
GCAGCACCTCGGCCAGCTCAACATGCTCCACCGACGATCCGGCCTCCCCCGACCCTC
CGACTCCAACGCCGTGTCCCTCGTCATGCGACGAATCCGAAAGGAGAACGTGGACG
CCGGCGAGCGAGCCAAGCAGGCCCTTGCCTTCGAGCGAACCGACTTCGACCAGGTC
CGATCCCTCATGGAGAACTCCGACCGATGCCAGGACATCCGAAACCTCGCCTTCCTT
GGCATCGCCTACAACACCCTCCTTCGAATCGCCGAGATCGCCCGAATCCGAGTCAAG
GACATCTCCCGAACCGACGGCGGCCGAATGCTCATCCACATCGGCCGAACCAAGAC
CCTCGTGTCCACCGCCGGCGTCGAGAAGGCCCTCTCCCTCGGCGTCACCAAGCTCGT
CGAGCGATGGATCTCCGTGTCCGGCGTCGCTGACGACCCCAACAACTACCTCTTCTG
CCGAGTCCGAAAGAACGGCGTCGCTGCTCCCTCCGCCACCTCCCAGCTCTCCACCCG
AGCCCTTGAGGGCATCTTCGAGGCCACCCACCGACTCATCTACGGCGCCAAGGACG
ACTCCGGCCAGCGATACCTCGCCTGGTCCGGCCACTCTGCTCGAGTCGGTGCCGCCC
GAGACATGGCCCGAGCCGGTGTCTCCATCCCCGAGATCATGCAGGCCGGCGGCTGG
ACCAACGTCAACATCGTCATGAACTACATCCGAAACCTCGACTCCGAGACTGGCGC
CATGGTCCGACTTCTTGAGGACGGCGACTGATGATCATATGATTACATTAATAGCTA
ATTACGTGTATCCGATATATATACTAATTACAATAGTACATATTAGAACATACAATA
GTTTTAATTAA
pLD125
(SEQ ID NO: 172)
TTAATTAATTCCGCTTAATGGAGTCCAAAAAGACCAACCTCTGCGCCTCGATCGACG
TGACCACAACCGCCGAGTTCCTTTCGCTCATCGACAAGCTCGGTCCCCACATCTGTC
TCGTGAAGACGCACATCGATATCATCTCAGACTTCAGCTACGAGGGCACGATTGAGC
CGTTGCTTGTGCTTGCAGAGCGCCACGGGTTCTTGATATTCGAGGACAGGAAGTTTG
CTGATATCGGAAACACCGTGATGTTGCAGTACACCTCGGGGGTATACCGGATCGCG
GCGTGGAGTGACATCACGAACGCGCACGGAGTGACTGGGAAGGGCGTCGTTGAAGG
GTTGAAACGCGGTGCGGAGGGGGTAGAAAAGGAAAGGGGCGTGTTGATGTTGGCG
GAGTTGTCGAGTAAAGGCTCGTTGGCGCATGGTGAATATACCCGTGAGACGATCGA
GATTGCGAAGAGTGATCGGGAGTTCGTGATTGGGTTCATCGCGCAGCGGGACATGG
GGGGTAGAGAAGAAGGGTTTGATTGGATCATCATGACGCCTGGTGTGGGGTTGGAT
GATAAAGGCGATGCGTTGGGCCAGCAGTATAGGACTGTTGATGAGGTGGTTCTGAC
TGGTACCGATGTGATTATTGTCGGGAGAGGGTTGTTTGGAAAAGGAAGAGACCCTG
AGGTGGAGGGAAAGAGATACAGGGATGCTGGATGGAAGGCATACTTGAAGAGAAC
TGGTCAGTTAGAATAAATATTGTAATAAATAGGTCTATATACATACACTAAGCTTCT
AGGACGTCATTGTAGTCTTCGAAGTTGTCTGCTAGTTTAGTTCTCATGATTTCGAAAA
CCAATAACGCAATGGATGTAGCAGGGATGGTGGTTAGTGCGTTCCTGACAAACCCA
GAGTACGCCGCCTCAAACCACGTCACATTCGCCCTTTGCTTCATCCGCATCACTTGCT
TGAAGGTATCCACGTACGAGTTGTAATACACCTTGAAGAACGGCTTCGTCTAGTTCG
GCATGGCAGATCATCATGCCTGCAGGAGCTCCAATTGTAATATTTCGGGAGAAATAT
CGTTGGGGTAAAACAACAGAGAGAGAGAGGGAGAGATGGTTCTGGTAGAATTATAA
TCTGGTTGTTGCAAATGCTACTGATCGACTCTGGCAATGTCTGTAGCTCGCTAGTTGT
ATGCAACTTAGGTGTTATGCATACACACGGTTATTCGGTTGAATTGTGGAGTAAAAA
TTGTCTGAGTTGTGTCTTAGCTACTGGCTGGCCCCCCGCGAAAGATAATCAAAATTA
CACTTGTGAATTTTTGCACACACACCGATTAACATTTCCCTTTTTTGTCCACCGATAC
ACGCTTGCCTCTTCTTATTTTCTCTGTGCTTCCCCCTCCTGTGACTTTTTCCACCATTG
ATATAAAATCAACTCCATTTCCCTAAAATCTCCCCAGATTCTAAAAACAACTTCTTCT
CTTCTGCTTTTCCTTATTTTTGTTATATTTATTTACCATCCCTTATTTTGAATAGTTATT
CCCCACTAACATTGTTCAAATCTTCACGACATAATGTTCTTGAAACACATTTTTGTTG
CTCTCGCTTTTGCCTTGTTAGCTGACGCTACCCCAGCCCAGAAGAGATCTCCCGGCTT
CGTTGCTTTAGACTTTGACATCGTCAAGGTTCAAAAGAACGTGACTGCCAACGACGA
CGCCGCTGCCATTGTTGCCAAGAGACAGACCAACCCAAGGGAAAACTTCCTTAAGT
GTTTTCTGCAGTACATCCCTAACAATGCAACAAACCTCAAGTTGGTGTACACTCAAA
ACAATCCACTCTATATGAGCGTGCTTAATAGCACAATCCACAACTTGCGCTTCACGT
CAGATACTACGCCTAAGCCACTAGTGATCGTTACACCATCACACGTCAGCCATATTC
AAGGAACGATCCTATGTCTGAAAAAGGTCGGGTTGCAAATCAGGACTCGATCAGGA
GGGCACGATAGTGAGGGAATGAGTTACATCTCGCAAGTACCCTTCGTGATAGTTGAC
TTGCGAAATATGCGGTCTATTAAAATTGACGTACATAGCCAGACCGCCTGGGTTGAA
GCAGGGGCAACCTTGGGTGAAGTTTATTACTGGGTCAATGAAAAAAACGAAAACCT
AAGTCTTGCTGCTGGATATTGCCCCACCGTTTGCGCGGGTGGTCATTTTGGAGGCGG
CGGATATGGTCCGTTGATGAGAAATTATGGACTTGCAGCAGACAATATTATAGATGC
CCACTTGGTGAACGTTCATGGAAAGGTCTTGGACCGTAAGTCCATGGGTGAAGATCT
TTTCTGGGCCTTGAGAGGTGGTGGAGCGGAATCGTTTGGCATCATCGTTGCCTGGAA
AATTAGGTTGGTTGCGGTCCCGAAGAGTACAATGTTCTCCGTGAAGAAGATTATGGA
AATACATGAGCTTGTCAAGTTAGTTAACAAGTGGCAAAATATCGCTTATAAGTATGA
TAAAGACTTGCTTTTGATGACTCATTTTATTACGCGAAACATAACCGATAACCAGGG
CAAGAACAAGACTGCTATTCACACGTACTTCTCCTCTGTATTTCTTGGAGGAGTAGA
CTCCTTAGTTGACTTGATGAACAAGAGTTTCCCAGAATTGGGGATTAAGAAGACAGA
TTGCAGACAATTATCGTGGATAGATACAATCATATTCTATAGCGGTGTCGTCAATTA
CGATACTGATAATTTTAATAAAGAAATCCTCCTAGATCGTTCAGCTGGGCAAAACGG
GGCATTCAAAATTAAATTGGATTATGTGAAGAAACCAATTCCAGAGCTGGTGTTTGT
TCAGATATTGGAAAAACTTTACGAAGAAGACATTGGCGCAGGTATGTACGCTTTGTA
TCCATATGGAGGCATTATGGACGAGATCTCAGAGCTGGCGATCCCCTTCCCGCACAG
AGCTGGGATACTCTACGAGCTATGGTACATCTGCTCTTGGGAGAAACAAGAAGACA
ACGAGAAACATCTCAATTGGATTCGGAACATATACAACTTTATGACCCCATACGTAT
CAAAAAACCCGCGCTTAGCATACTTGAATTACAGAGACTTAGATATCGGTATCAATG
ATCCTAAGAATCCTAACAATTACACCCAAGCCCGTATTTGGGGTGAGAAATATTTCG
GCAAGAATTTTGACAGATTAGTTAAGGTCAAAACACTCGTGGACCCCAACAACTTTT
TCCGAAACGAGCAGTCGATTCCACCACTACCCAGGCATAGACACTGAGAGTGACTC
TTTTGATAAGAGTCGCAAATTTGATTTCATAAGTATATATTCATTATGTAAAGTAGTA
AATGGAAAATTCATTAAAAAAAAAGCAAATTTCCGTTGTATGCATACTCCGAACAC
AAAACTAGCCCCGGAAAAACCCTTAGTTGATAGTTGCGAATTTAGGTCGACCATATG
CGACGGGTACAACGAGAATTGTATTGAATTGATCAAGAACATGATCTTGGTGTTACA
GAACATCAAGTTCTTGGACCAGACTGAGAATGCACAGATATACAAGGCGTCATGTG
ATAAAATGGATGAGATTTATCCACAATTGAAGAAAGAGTTTATGGAAAGTGGTCAA
CCAGAAGCTAAACAGGAAGAAGCAAACGAAGAGGTGAAACAAGAAGAAGAAGGTA
AATAAGTATTTTGTATTATATAACAAACAAAGTAAGGAATACAGATTTATACAATAA
ATTGCCATACTAGTCACGTGAGATATCTCATCCATTCCCCAACTCCCAAGAAAATAA
AAAAGTGAAAAATAAAATCAAACCCAAAGATCAACCTCCCCATCATCATCGTCATC
AAACCCCCAGCTCAATTCGCAATGGTTAGCACAAAAACATACACAGAAAGGGCATC
AGCACACCCCTCCAAGGTTGCCCAACGTTTATTAATTAAAGGCTAGGTGGAGGCTCA
GTGATGATAAGTCTGCGATGGTGGATGCATGTGTCATGGTCATAGCTGTTTCCTGTG
TGAAATTGTTATCCGCTCAGAGGGCACAATCCTATTCCGCGCTATCCGACAATCTCC
AAGACATTAGGTGGAGTTCAGTTCGGCGTATGGCATATGTCGCTGGAAAGAACATG
TGAGCAAAAGGCCAGCAAAAGGCCAGGAACCGTAAAAAGGCCGCGTTGCTGGCGTT
TTTCCATAGGCTCCGCCCCCCTGACGAGCATCACAAAAATCGACGCTCAAGTCAGAG
GTGGCGAAACCCGACAGGACTATAAAGATACCAGGCGTTTCCCCCTGGAAGCTCCC
TCGTGCGCTCTCCTGTTCCGACCCTGCCGCTTACCGGATACCTGTCCGCCTTTCTCCC
TTCGGGAAGCGTGGCGCTTTCTCATAGCTCACGCTGTAGGTATCTCAGTTCGGTGTA
GGTCGTTCGCTCCAAGCTGGGCTGTGTGCACGAACCCCCCGTTCAGCCCGACCGCTG
CGCCTTATCCGGTAACTATCGTCTTGAGTCCAACCCGGTAAGACACGACTTATCGCC
ACTGGCAGCAGCCACTGGTAACAGGATTAGCAGAGCGAGGTATGTAGGCGGTGCTA
CAGAGTTCTTGAAGTGGTGGCCTAACTACGGCTACACTAGAAGAACAGTATTTGGTA
TCTGCGCTCTGCTGAAGCCAGTTACCTTCGGAAAAAGAGTTGGTAGCTCTTGATCCG
GCAAACAAACCACCGCTGGTAGCGGTGGTTTTTTTGTTTGCAAGCAGCAGATTACGC
GCAGAAAAAAAGGATCTCAAGAAGATCCTTTGATCTTTTCTACGGGGTCTGACGCTC
TATTCAACAAAGCCGCCGTCCCGTCAAGTCAGCGTAAATGGGTAGGGGGCTTCAAA
TCGTCCGCTCTGCCAGTGTTACAACCAATTAACAAATTCTGATTAGAAAAACTCATC
GAGCATCAAATGAAACTGCAATTTATTCATATCAGGATTATCAATACCATATTTTTG
AAAAAGCCGTTTCTGTAATGAAGGAGAAAACTCACCGAGGCAGTTCCATAGGATGG
CAAGATCCTGGTATCGGTCTGCGATTCCGACTCGTCCAACATCAATACAACCTATTA
ATTTCCCCTCGTCAAAAATAAGGTTATCAAGTGAGAAATCACCATGAGTGACGACTG
AATCCGGTGAGAATGGCAAAAGCTTATGCATTTCTTTCCAGACTTGTTCAACAGGCC
AGCCATTACGCTCGTCATCAAAATCACTCGCATCAACCAAACCGTTATTCATTCGTG
ATTGCGCCTGAGCGAGACGAAATACGCGATCGCTGTTAAAAGGACAATTACAAACA
GGAATCGAATGCAACCGGCGCAGGAACACTGCCAGCGCATCAACAATATTTTCACC
TGAATCAGGATATTCTTCTAATACCTGGAATGCTGTTTTCCCGGGGATCGCAGTGGT
GAGTAACCATGCATCATCAGGAGTACGGATAAAATGCTTGATGGTCGGAAGAGGCA
TAAATTCCGTCAGCCAGTTTAGTCTGACCATCTCATCTGTAACATCATTGGCAACGCT
ACCTTTGCCATGTTTCAGAAACAACTCTGGCGCATCGGGCTTCCCATACAATCGATA
GATTGTCGCACCTGATTGCCCGACATTATCGCGAGCCCATTTATACCCATATAAATC
AGCATCCATGTTGGAATTTAATCGCGGCCTCGAGCAAGACGTTTCCCGTTGAATATG
GCTCATAACACCCCTTGTATTACTGTTTATGTAAGCAGACAGTTTTATTGTTCATGAT
GATATATTTTTATCTTGTGCAATGTAACATCAGAGATTTTGAGACACAACGTGGCTTT
CCCCCGCCGCTCTAGAACTAGTGGATCCAAATAAAACGAAAGGCTCAGTCGAAAGA
CTGGGCCTTTCGTTTTATCTGTTGTTTGTCGCATTATACGAGACGTCCAGGTTGGGAT
ACCTGAAACAAAACCCATCGTACGGCCAAGGAAGTCTCCAATAACTGTGATCCACC
ACAAGCGCCAGGGTTTTCCCAGTCACGACGTTGTAAAACGACGGCCAGTCATGCAT
AATCCGCACGCATCTGGAATAAGGAAGTGCCATTCCGCCTGACCT
pLD127
(SEQ ID NO: 173)
TTAATTAATTCCGCTTAATGGAGTCCAAAAAGACCAACCTCTGCGCCTCGATCGACG
TGACCACAACCGCCGAGTTCCTTTCGCTCATCGACAAGCTCGGTCCCCACATCTGTC
TCGTGAAGACGCACATCGATATCATCTCAGACTTCAGCTACGAGGGCACGATTGAGC
CGTTGCTTGTGCTTGCAGAGCGCCACGGGTTCTTGATATTCGAGGACAGGAAGTTTG
CTGATATCGGAAACACCGTGATGTTGCAGTACACCTCGGGGGTATACCGGATCGCG
GCGTGGAGTGACATCACGAACGCGCACGGAGTGACTGGGAAGGGCGTCGTTGAAGG
GTTGAAACGCGGTGCGGAGGGGGTAGAAAAGGAAAGGGGCGTGTTGATGTTGGCG
GAGTTGTCGAGTAAAGGCTCGTTGGCGCATGGTGAATATACCCGTGAGACGATCGA
GATTGCGAAGAGTGATCGGGAGTTCGTGATTGGGTTCATCGCGCAGCGGGACATGG
GGGGTAGAGAAGAAGGGTTTGATTGGATCATCATGACGCCTGGTGTGGGGTTGGAT
GATAAAGGCGATGCGTTGGGCCAGCAGTATAGGACTGTTGATGAGGTGGTTCTGAC
TGGTACCGATGTGATTATTGTCGGGAGAGGGTTGTTTGGAAAAGGAAGAGACCCTG
AGGTGGAGGGAAAGAGATACAGGGATGCTGGATGGAAGGCATACTTGAAGAGAAC
TGGTCAGTTAGAATAAATATTGTAATAAATAGGTCTATATACATACACTAAGCTTCT
AGGACGTCATTGTAGTCTTCGAAGTTGTCTGCTAGTTTAGTTCTCATGATTTCGAAAA
CCAATAACGCAATGGATGTAGCAGGGATGGTGGTTAGTGCGTTCCTGACAAACCCA
GAGTACGCCGCCTCAAACCACGTCACATTCGCCCTTTGCTTCATCCGCATCACTTGCT
TGAAGGTATCCACGTACGAGTTGTAATACACCTTGAAGAACGGCTTCGTCTAGTTCG
GCATGGCAGATCATCATGCCTGCAGGAGCTCCAATTGTAATATTTCGGGAGAAATAT
CGTTGGGGTAAAACAACAGAGAGAGAGAGGGAGAGATGGTTCTGGTAGAATTATAA
TCTGGTTGTTGCAAATGCTACTGATCGACTCTGGCAATGTCTGTAGCTCGCTAGTTGT
ATGCAACTTAGGTGTTATGCATACACACGGTTATTCGGTTGAATTGTGGAGTAAAAA
TTGTCTGAGTTGTGTCTTAGCTACTGGCTGGCCCCCCGCGAAAGATAATCAAAATTA
CACTTGTGAATTTTTGCACACACACCGATTAACATTTCCCTTTTTTGTCCACCGATAC
ACGCTTGCCTCTTCTTATTTTCTCTGTGCTTCCCCCTCCTGTGACTTTTTCCACCATTG
ATATAAAATCAACTCCATTTCCCTAAAATCTCCCCAGATTCTAAAAACAACTTCTTCT
CTTCTGCTTTTCCTTATTTTTGTTATATTTATTTACCATCCCTTATTTTGAATAGTTATT
CCCCACTAACATTGTTCAAATCTTCACGACataATGCAATTGTCCTTGTCGGTTTTATC
AACCGTTGCCACGGCCTTGTTGTCCCTAACCACCGCCGTCGATGCTAAGTCCCACAA
CATCAAGTTGTCCAAGTTGTCCAACGAAGAAACATTGGACGCCTCCACATTCCAAGA
ATACACGAGCTCCTTGGCCAACAAGTACATGAACTTGTTCAACGCCGCTCACGGTAA
CCCAACCAGCTTTGGCTTGCAACACGTCTTGTCCAACCAAGAAGCTGAAGTCCCATT
CGTTACCCCACAAAAGGGTggcAACCCAAGGGAAAACTTCCTTAAGTGTTTTCTGCAG
TACATCCCTAACAATGCAACAAACCTCAAGTTGGTGTACACTCAAAACAATCCACTC
TATATGAGCGTGCTTAATAGCACAATCCACAACTTGCGCTTCACGTCAGATACTACG
CCTAAGCCACTAGTGATCGTTACACCATCACACGTCAGCCATATTCAAGGAACGATC
CTATGTCTGAAAAAGGTCGGGTTGCAAATCAGGACTCGATCAGGAGGGCACGATAG
TGAGGGAATGAGTTACATCTCGCAAGTACCCTTCGTGATAGTTGACTTGCGAAATAT
GCGGTCTATTAAAATTGACGTACATAGCCAGACCGCCTGGGTTGAAGCAGGGGCAA
CCTTGGGTGAAGTTTATTACTGGGTCAATGAAAAAAACGAAAACCTAAGTCTTGCTG
CTGGATATTGCCCCACCGTTTGCGCGGGTGGTCATTTTGGAGGCGGCGGATATGGTC
CGTTGATGAGAAATTATGGACTTGCAGCAGACAATATTATAGATGCCCACTTGGTGA
ACGTTCATGGAAAGGTCTTGGACCGTAAGTCCATGGGTGAAGATCTTTTCTGGGCCT
TGAGAGGTGGTGGAGCGGAATCGTTTGGCATCATCGTTGCCTGGAAAATTAGGTTGG
TTGCGGTCCCGAAGAGTACAATGTTCTCCGTGAAGAAGATTATGGAAATACATGAG
CTTGTCAAGTTAGTTAACAAGTGGCAAAATATCGCTTATAAGTATGATAAAGACTTG
CTTTTGATGACTCATTTTATTACGCGAAACATAACCGATAACCAGGGCAAGAACAAG
ACTGCTATTCACACGTACTTCTCCTCTGTATTTCTTGGAGGAGTAGACTCCTTAGTTG
ACTTGATGAACAAGAGTTTCCCAGAATTGGGGATTAAGAAGACAGATTGCAGACAA
TTATCGTGGATAGATACAATCATATTCTATAGCGGTGTCGTCAATTACGATACTGAT
AATTTTAATAAAGAAATCCTCCTAGATCGTTCAGCTGGGCAAAACGGGGCATTCAAA
ATTAAATTGGATTATGTGAAGAAACCAATTCCAGAGCTGGTGTTTGTTCAGATATTG
GAAAAACTTTACGAAGAAGACATTGGCGCAGGTATGTACGCTTTGTATCCATATGGA
GGCATTATGGACGAGATCTCAGAGCTGGCGATCCCCTTCCCGCACAGAGCTGGGAT
ACTCTACGAGCTATGGTACATCTGCTCTTGGGAGAAACAAGAAGACAACGAGAAAC
ATCTCAATTGGATTCGGAACATATACAACTTTATGACCCCATACGTATCAAAAAACC
CGCGCTTAGCATACTTGAATTACAGAGACTTAGATATCGGTATCAATGATCCTAAGA
ATCCTAACAATTACACCCAAGCCCGTATTTGGGGTGAGAAATATTTCGGCAAGAATT
TTGACAGATTAGTTAAGGTCAAAACACTCGTGGACCCCAACAACTTTTTCCGAAACG
AGCAGTCGATTCCACCACTACCCAGGCATAGACACTGAGAGTGACTCTTTTGATAAG
AGTCGCAAATTTGATTTCATAAGTATATATTCATTATGTAAAGTAGTAAATGGAAAA
TTCATTAAAAAAAAAGCAAATTTCCGTTGTATGCATACTCCGAACACAAAACTAGCC
CCGGAAAAACCCTTAGTTGATAGTTGCGAATTTAGGTCGACCATATGCGACGGGTAC
AACGAGAATTGTATTGAATTGATCAAGAACATGATCTTGGTGTTACAGAACATCAAG
TTCTTGGACCAGACTGAGAATGCACAGATATACAAGGCGTCATGTGATAAAATGGA
TGAGATTTATCCACAATTGAAGAAAGAGTTTATGGAAAGTGGTCAACCAGAAGCTA
AACAGGAAGAAGCAAACGAAGAGGTGAAACAAGAAGAAGAAGGTAAATAAGTATT
TTGTATTATATAACAAACAAAGTAAGGAATACAGATTTATACAATAAATTGCCATAC
TAGTCACGTGAGATATCTCATCCATTCCCCAACTCCCAAGAAAATAAAAAAGTGAA
AAATAAAATCAAACCCAAAGATCAACCTCCCCATCATCATCGTCATCAAACCCCCA
GCTCAATTCGCAATGGTTAGCACAAAAACATACACAGAAAGGGCATCAGCACACCC
CTCCAAGGTTGCCCAACGTTTATTAATTAAAGGCTAGGTGGAGGCTCAGTGATGATA
AGTCTGCGATGGTGGATGCATGTGTCATGGTCATAGCTGTTTCCTGTGTGAAATTGTT
ATCCGCTCAGAGGGCACAATCCTATTCCGCGCTATCCGACAATCTCCAAGACATTAG
GTGGAGTTCAGTTCGGCGTATGGCATATGTCGCTGGAAAGAACATGTGAGCAAAAG
GCCAGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCGAAA
CCCGACAGGACTATAAAGATACCAGGCGTTTCCCCCTGGAAGCTCCCTCGTGCGCTC
TCCTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTCGCT
CCAAGCTGGGCTGTGTGCACGAACCCCCCGTTCAGCCCGACCGCTGCGCCTTATCCG
GTAACTATCGTCTTGAGTCCAACCCGGTAAGACACGACTTATCGCCACTGGCAGCAG
CCACTGGTAACAGGATTAGCAGAGCGAGGTATGTAGGCGGTGCTACAGAGTTCTTG
AAGTGGTGGCCTAACTACGGCTACACTAGAAGAACAGTATTTGGTATCTGCGCTCTG
CTGAAGCCAGTTACCTTCGGAAAAAGAGTTGGTAGCTCTTGATCCGGCAAACAAAC
CACCGCTGGTAGCGGTGGTTTTTTTGTTTGCAAGCAGCAGATTACGCGCAGAAAAAA
AGGATCTCAAGAAGATCCTTTGATCTTTTCTACGGGGTCTGACGCTCTATTCAACAA
AGCCGCCGTCCCGTCAAGTCAGCGTAAATGGGTAGGGGGCTTCAAATCGTCCGCTCT
GCCAGTGTTACAACCAATTAACAAATTCTGATTAGAAAAACTCATCGAGCATCAAAT
GAAACTGCAATTTATTCATATCAGGATTATCAATACCATATTTTTGAAAAAGCCGTT
TCTGTAATGAAGGAGAAAACTCACCGAGGCAGTTCCATAGGATGGCAAGATCCTGG
TATCGGTCTGCGATTCCGACTCGTCCAACATCAATACAACCTATTAATTTCCCCTCGT
CAAAAATAAGGTTATCAAGTGAGAAATCACCATGAGTGACGACTGAATCCGGTGAG
AATGGCAAAAGCTTATGCATTTCTTTCCAGACTTGTTCAACAGGCCAGCCATTACGC
TCGTCATCAAAATCACTCGCATCAACCAAACCGTTATTCATTCGTGATTGCGCCTGA
GCGAGACGAAATACGCGATCGCTGTTAAAAGGACAATTACAAACAGGAATCGAATG
CAACCGGCGCAGGAACACTGCCAGCGCATCAACAATATTTTCACCTGAATCAGGAT
ATTCTTCTAATACCTGGAATGCTGTTTTCCCGGGGATCGCAGTGGTGAGTAACCATG
CATCATCAGGAGTACGGATAAAATGCTTGATGGTCGGAAGAGGCATAAATTCCGTC
AGCCAGTTTAGTCTGACCATCTCATCTGTAACATCATTGGCAACGCTACCTTTGCCAT
GTTTCAGAAACAACTCTGGCGCATCGGGCTTCCCATACAATCGATAGATTGTCGCAC
CTGATTGCCCGACATTATCGCGAGCCCATTTATACCCATATAAATCAGCATCCATGT
TGGAATTTAATCGCGGCCTCGAGCAAGACGTTTCCCGTTGAATATGGCTCATAACAC
CCCTTGTATTACTGTTTATGTAAGCAGACAGTTTTATTGTTCATGATGATATATTTTT
ATCTTGTGCAATGTAACATCAGAGATTTTGAGACACAACGTGGCTTTCCCCCGCCGC
TCTAGAACTAGTGGATCCAAATAAAACGAAAGGCTCAGTCGAAAGACTGGGCCTTT
CGTTTTATCTGTTGTTTGTCGCATTATACGAGACGTCCAGGTTGGGATACCTGAAAC
AAAACCCATCGTACGGCCAAGGAAGTCTCCAATAACTGTGATCCACCACAAGCGCC
AGGGTTTTCCCAGTCACGACGTTGTAAAACGACGGCCAGTCATGCATAATCCGCACG
CATCTGGAATAAGGAAGTGCCATTCCGCCTGACCT
pLD131
(SEQ ID NO: 174)
AGGCTAGGTGGAGGCTCAGTGATGATAAGTCTGCGATGGTGGATGCATGTGTCATG
GTCATAGCTGTTTCCTGTGTGAAATTGTTATCCGCTCAGAGGGCACAATCCTATTCCG
CGCTATCCGACAATCTCCAAGACATTAGGTGGAGTTCAGTTCGGCGTATGGCATATG
TCGCTGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAAAA
TCGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCC
GTTCAGCCCGACCGCTGCGCCTTATCCGGTAACTATCGTCTTGAGTCCAACCCGGTA
AGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAG
AAGAACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTT
CTACGGGGTCTGACGCTCTATTCAACAAAGCCGCCGTCCCGTCAAGTCAGCGTAAAT
GGGTAGGGGGCTTCAAATCGTCCGCTCTGCCAGTGTTACAACCAATTAACAAATTCT
GATTAGAAAAACTCATCGAGCATCAAATGAAACTGCAATTTATTCATATCAGGATTA
TCAATACCATATTTTTGAAAAAGCCGTTTCTGTAATGAAGGAGAAAACTCACCGAGG
CAGTTCCATAGGATGGCAAGATCCTGGTATCGGTCTGCGATTCCGACTCGTCCAACA
TCAATACAACCTATTAATTTCCCCTCGTCAAAAATAAGGTTATCAAGTGAGAAATCA
CCATGAGTGACGACTGAATCCGGTGAGAATGGCAAAAGCTTATGCATTTCTTTCCAG
ACTTGTTCAACAGGCCAGCCATTACGCTCGTCATCAAAATCACTCGCATCAACCAAA
CCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATC
AACAATATTTTCACCTGAATCAGGATATTCTTCTAATACCTGGAATGCTGTTTTCCCG
GGGATCGCAGTGGTGAGTAACCATGCATCATCAGGAGTACGGATAAAATGCTTGAT
GGTCGGAAGAGGCATAAATTCCGTCAGCCAGTTTAGTCTGACCATCTCATCTGTAAC
ATCATTGGCAACGCTACCTTTGCCATGTTTCAGAAACAACTCTGGCGCATCGGGCTT
CCCATACAATCGATAGATTGTCGCACCTGATTGCCCGACATTATCGCGAGCCCATTT
ATACCCATATAAATCAGCATCCATGTTGGAATTTAATCGCGGCCTCGAGCAAGACGT
TTCCCGTTGAATATGGCTCATAACACCCCTTGTATTACTGTTTATGTAAGCAGACAGT
TTTATTGTTCATGATGATATATTTTTATCTTGTGCAATGTAACATCAGAGATTTTGAG
ACACAACGTGGCTTTCCCCCGCCGCTCTAGAACTAGTGGATCCAAATAAAACGAAA
GGCTCAGTCGAAAGACTGGGCCTTTCGTTTTATCTGTTGTTTGTCGCATTATACGAGA
CGTCCAGGTTGGGATACCTGAAACAAAACCCATCGTACGGCCAAGGAAGTCTCCAA
TAACTGTGATCCACCACAAGCGCCAGGGTTTTCCCAGTCACGACGTTGTAAAACGAC
GGCCAGTCATGCATAATCCGCACGCATCTGGAATAAGGAAGTGCCATTCCGCCTGA
CCTGATCCCAATATTACACCCAAGTAGCATGCATAAGCTAAAAGTAACTCGCAGCG
CACACCGTGCAGATTCATAAGTCTATGATTAATTGAACGCCAATAACCCGGCTTACT
ACAAGTACAAGTAGGTATACATAGCGGTAATGAATCATTAGAAAAATAAAAAACAA
AAAAAAACAAAACAAACTGTTGTGGATGCATCAACAGTAGTACATAGTTGTACGAT
GTACTTGTACTTGTAAAAGCAAAAATGTACAATATCTCAGGGAGCGCAACTTTTACG
TTCGAAGAACAATGTACCGCATACCGCATTCTAGATTCTGCGGAACGTCTAACCTGG
AAATACGATTTTTTATTTCTTTCATTTTTTTTGCTTCTTCAAAAGTATGGTAATTTCCT
ACCATTACAGTTGACACTGAACGAGGGGGGATTGAATTTAAGCAAAAAATTAAATC
AAAATACCTTTATGTATCCAGCCCATGTAATAAACAAAAGGATTATATAACAAGAA
ATAAATATATACCTTTAATGGATCATTAGAATAAAAATAAATACGAGAAGCACACC
AGAGAAGCTTTTTGATTGCCACTATACCGCTACTTTGGTATATCTTATTATAATTGTT
GAATTTGCAAGATAGAATGTCATTCATTGGAGAGAAATCCAAGGAATATGTGGGAT
GAAATGACTAGAAGTATGAACAATGAGAATAGTACATACTTGTACCTGTATTTCTAG
AAGAGAGAAAGACAGTTGAGTGTGTGATTCTCGTCCAATAATAATCTCAATAGTAA
CGTGTGAATAGCTGTTCTTTGATAGTTGATATTTCTCGATGACTATTTATGTTGTACA
AGGGATTTTTTTCGTTGCTGTTGATTTCGAATTAGGCAATGCAGATATCATTTATGCT
ATCCATATTTAAGATTTCCCATACGCATTTATAACATTTATTCTACATAAATTGTTAA
ATGAACGAACTGCCATTATAAATTGTTTCCTAAATAGGAAGTGTTTTTCATAAAGCA
AGTAAGTTGTCTAATAATACTAAGTAATAAAAATAAGTTCATACAATATATTTTGAG
AACATCATTTGGAGGCGGTAGATGGAGTCTGTTTATTATTAAACAATGCGAGATGAC
CCCTTAAATATTGAGAACATCAGTTGGAGGCGGCAGATGGAGTCTGTCTATTTAGCA
ATGGGACATGACTGTCAGTATCATCATATGTATATATATAATACATATAATATTATA
TAACACGATTTTTTTAAATTATTGGCCCGAAAATTAATCAGTGTAGACTGGATCCTC
GAGAACCATTTAATTAAGATCGTCGACGATTCCGCCAAGTGAGACTGGCGATCGGG
AGAAGGGTTGGTGGTCATGGGGGATAGAATTTGTACAAGTGGAAAAACCACTACGA
GTAGCGGATTTGATACCACAAGTAGCAGAGATATACAGCAATGGTGGGAGTGCAAG
TATCGGAATGTACTGTACCTCCTGTACTCGTACTCGTACGGCACTCGTAGAAACGGG
GCAATACGGGGGAGAAGCGATCGCCCGTCTGTTCAATCGCCACAAGTCCGAGTAAT
GCTTGAGTATCGAAGTCTTGTACCTCCCTGTCAATCATGGCACCACTGGTCTTGACTT
GTCTATTCATACTGGACAAGCGCCAGAGTTAAGCTTGTAGCGAATTTCGCCCTCGGA
CATCACCCCATACGACGGACACACATGCCCGACAAACAGCCTCTCTTATTGTAGCTG
AAAGTATATTGAATGTGAACGTGTACAATATCAGGTACCAGCGGGAGGTTACGGCC
AAGGTGATACCGGAATAACCCTGGCTTGGAGATGGTCGGTCCATTGTACTGAAGTGT
CCGTGTCGTTTCCGTCACTGCCCCAATTGGACATGTTTGTTTTTCCGATCTTTCGGGC
GCCCTCTCCTTGTCTCCTTGTCTGTCTCCTGGACTGTTGCTACCCCATTTCTTTGGCCT
CCATTGGTTCCTCCCCGTCTTTCACGTCGTCTATGGTTGCATGGTTTCCCTTATACTTT
TCCCCACAGTCACATGTTATGGAGGGGTCTAGATGGAGGCCTAATTTTGACGTGCAA
GGGGCGAATTGGGGCGAGAAACACGTCGTGGACATGGTGCAAGGCCCGCAGGGTTG
ATTCGACGCTTTTCCGCGAAAAAAACAAGTCCAAATACCCCCGTTTATTCTCCCTCG
GCTCTCGGTATTTCACATGAAAACTATAACCTAGACTACACGGGCAACCTTAACCCC
AGAGTATACTTATATACCAAAGGGATGGGTCCTCAAAAATCACACAAGCAACGACG
CCATGGGCCTCTCTCTAGTATGTACCTTCTCTTTCCAGACCAACTATCACACTCTACT
GAACCCCCATAACAAGAACCCTAAAAATTCTCTTCTCAGTTACCAGCACCCCAAGAC
GCCTATCATTAAGTCCTCCTACGACAACTTTCCCTCTAAGTACTGCCTGACCAAAAA
CTTCCATCTCCTGGGACTGAACTCTCATAACAGAATTAGTAGCCAGTCCCGATCTAT
CCGAGCTGGCTCTGACCAGATTGAGGGCTCCCCTCACCATGAATCCGACAACAGCAT
CGCTACCAAGATTTTGAATTTTGGTCACACATGCTGGAAGCTCCAGCGACCGTACGT
CGTGAAGGGTATGATCTCGATTGCCTGTGGACTGTTCGGACGTGAGCTTTTTAATAA
TCGACACTTGTTTTCATGGGGCCTCATGTGGAAGGCTTTTTTCGCCCTCGTGCCCATT
CTGTCTTTCAACTTCTTTGCCGCTATTATGAACCAAATCTACGACGTTGATATTGATA
GGATCAACAAGCCTGACCTGCCGCTCGTCTCGGGGGAGATGTCTATCGAGACAGCG
TGGATTCTTTCGATTATCGTCGCGCTGACTGGCCTTATCGTTACCATAAAGTTGAAGT
CTGCACCCCTCTTCGTGTTTATCTACATTTTCGGTATTTTTGCTGGATTCGCGTACTCC
GTTCCCCCTATCAGATGGAAGCAGTACCCCTTTACTAACTTTCTGATTACTATCAGCA
GCCACGTCGGTTTAGCCTTTACCTCATATTCGGCCACCACCAGTGCACTGGGCCTCC
CCTTCGTCTGGCGACCTGCATTTTCATTCATCATCGCCTTCATGACTGTGATGGGTAT
GACCATCGCTTTCGCTAAGGACATCTCCGACATCGAGGGTGATGCTAAATATGGAGT
GTCCACCGTGGCCACTAAGCTGGGAGCCCGGAACATGACGTTCGTCGTCTCTGGTGT
TCTGCTCCTTAACTACTTGGTTTCGATCTCCATTGGCATTATCTGGCCACAAGTCTTC
AAGTCCAACATTATGATTCTGTCCCACGCCATTCTTGCCTTTTGCCTGATCTTCCAGA
CACGCGAACTCGCTCTCGCTAACTACGCCTCCGCCCCATCGCGACAGTTCTTCGAGT
TCATCTGGCTGCTTTACTACGCCGAGTACTTCGTTTACGTGTTCATCTAATAAGAGTA
GGCAATTAACAGATAGTTTGCCGGTGATAATTCTCTTAACCTCCCACACTCCTTTGAC
ATAACGATTTATGTAACGAAACTGAAATTTGACCAGATATTGTTGTAAATAGAAAAT
CTGGCTTGTAGGTGGCAAAATGCGGCGTCTTTGTTCATCAATTCCCTCTGTGACTACT
CGTCATCCCTTTATGTTCGACTGTCGTATTTCTTATTTTCCATACATATGCAAGTGAG
ATGCCCGTGTCCGAATTCGCTATGGATCCATAGCGTCGACACCATATCATATAAAAC
TAACAATGCATTGAATTAATACGAAGACTACCCGTTGCTATCTCCACACCGTTATCT
CCACGGTCCAAAGGCTGCTCAATGTGCTGCATACGTAACGTGGGGTGCAACCTTGAG
CACATAGTACTTTCCGAAAACCGGCGATAATTAAGTGTGCACTCCAACTTTTCACAC
TGAGCATAAAATGTGGAGAAGAAATCACACTAAAAAGTCAGGTAGACTGGAAAATG
CGCCATGAAATAATATCTCTTGCTACAGTAATGCCCAGCATCGAGGGGTATTGTGTC
ACCAACACTATAGTGGCAGCTGAAGCGCTCGTGATTGTAGTATGAGTCTTTATTGGT
GATGGGAAGAGTTCACTCAATATTCTCGTTACTGCCAAAACACCACGGTAATCGGCC
AGACACCATGGATGTAGATCACCAAGCCTGTGAATGTTATTCGAGCTAAAATGCAC
ATGGTTGGTGAAAGGAGTAGTTGCTGTCGAATTCCGTCGTCGCCTGAGTCATCATTT
ATTTACCAGTTGGCCACAAACCCTTGACGATCTCGTATGTCCCCTCCGACATACTCC
CGGCCGGCTGGGGTACGTTCGATAGCGCTATCGGCATCGACAAGGTTTGGGTCCCTA
GCCGATACCGCACTACCTGAGTCACAATCTTCGGAGGTTTAGTCTTCCACATAGCAC
GGGCAAAAGTGCGTATATATACAAGAGCGTTTGCCAGCCACAGATTTTCACTCCACA
CACCACATCACACATACAACCACACACATCCACAATGGAACCCGAAACTAAGAAGA
CCAAGACTGACTCCAAGAAGATTGTTCTTCTCGGCGGCGACTTCTGTGGCCCCGAGG
TGATTGCCGAGGCCGTCAAGGTGCTCAAGTCTGTTGCTGAGGCCTCCGGCACCGAGT
TTGTGTTTGAGGACCGACTCATTGGAGGAGCTGCCATTGAGAAGGAGGGCGAGCCC
ATCACCGACGCTACTCTCGACATCTGCCGAAAGGCTGACTCTATTATGCTCGGTGCT
GTCGGAGGCGCTGCCAACACCGTATGGACCACTCCCGACGGACGAACCGACGTGCG
ACCCGAGCAGGGTCTTCTCAAGCTGCGAAAGGACCTGAACCTGTACGCCAACCTGC
GACCCTGCCAGCTGCTGTCGCCCAAGCTCGCCGATCTCTCCCCCATCCGAAACGTTG
AGGGCACCGACTTCATCATTGTCCGAGAGCTCGTCGGAGGTATCTACTTTGGAGAGC
GAAAGGAGGATGACGGATCTGGCGTCGCTTCCGACACTGAGACTTACTCCGTTCCTG
AGGTTGAGCGAATTGCCCGAATGGCCGCCTTCCTGGCCCTTCAGCACAACCCCCCTC
TTCCCGTGTGGTCCCTTGACAAGGCCAACGTGCTGGCCTCCTCTCGACTTTGGCGAA
AGACCGTCACCCGAGTCCTCAAGGACGAATTCCCCCAGCTGGAGCTCAACCACCAG
CTGATCGACTCGGCCGCCATGATCCTCATCAAGCAGCCCTCCAAGATGAATGGTATC
ATCATCACCACCAACATGTTTGGCGATATCATCTCCGACGAGGCCTCCGTCATCCCC
GGTTCTCTGGGTCTGCTGCCCTCCGCCTCTCTGGCTTCTCTGCCCGACACCAACGAGG
CGTTCGGTCTGTACGAGCCCTGTCACGGATCTGCCCCCGATCTCGGCAAGCAGAAGG
TCAACCCCATTGCCACCATTCTGTCTGCCGCCATGATGCTCAAGTTCTCTCTTAACAT
GAAGCCCGCCGGTGACGCTGTTGAGGCTGCCGTCAAGGAGTCCGTCGAGGCTGGTA
TCACTACCGCCGATATCGGAGGCTCCTCCTCCACCTCCGAGGTCGGAGACTTTGTTG
CCAACAAGGTCAAGGAGCTGCTCAAGAAGGAGTAAGTCGTTTCTACGACGCATTGA
TGGAAGGAGCAAACTGACGCGCCTGCGGGTTGGTCTACCGGCAGGGTCCGCTAGTG
TATAAGACTCTATAAAAAGGGCCCAGCCCTGCTAATGAAATGATGATTTATAATTTA
CCGGTGTAGCAACCTTGACTAGAAGAAGCAGATTGGGTGTGTTTGTAGTGGAGGAC
AGTGGTACGTTTTAATTAA
pLD132
(SEQ ID NO: 175)
TTAATTAAGATCGTCGACGATTCCGCCAAGTGAGACTGGCGATCGGGAGAAGGGTT
GGTGGTCATGGGGGATAGAATTTGTACAAGTGGAAAAACCACTACGAGTAGCGGAT
TTGATACCACAAGTAGCAGAGATATACAGCAATGGTGGGAGTGCAAGTATCGGAAT
GTACTGTACCTCCTGTACTCGTACTCGTACGGCACTCGTAGAAACGGGGCAATACGG
GGGAGAAGCGATCGCCCGTCTGTTCAATCGCCACAAGTCCGAGTAATGCTTGAGTAT
CGAAGTCTTGTACCTCCCTGTCAATCATGGCACCACTGGTCTTGACTTGTCTATTCAT
ACTGGACAAGCGCCAGAGTTAAGCTTGTAGCGAATTTCGCCCTCGGACATCACCCCA
TACGACGGACACACATGCCCGACAAACAGCCTCTCTTATTGTAGCTGAAAGTATATT
GAATGTGAACGTGTACAATATCAGGTACCAGCGGGAGGTTACGGCCAAGGTGATAC
CGGAATAACCCTGGCTTGGAGATGGTCGGTCCATTGTACTGAAGTGTCCGTGTCGTT
TCCGTCACTGCCCCAATTGGACATGTTTGTTTTTCCGATCTTTCGGGCGCCCTCTCCT
TGTCTCCTTGTCTGTCTCCTGGACTGTTGCTACCCCATTTCTTTGGCCTCCATTGGTTC
CTCCCCGTCTTTCACGTCGTCTATGGTTGCATGGTTTCCCTTATACTTTTCCCCACAGT
CACATGTTATGGAGGGGTCTAGATGGAGGCCTAATTTTGACGTGCAAGGGGCGAAT
TGGGGCGAGAAACACGTCGTGGACATGGTGCAAGGCCCGCAGGGTTGATTCGACGC
TTTTCCGCGAAAAAAACAAGTCCAAATACCCCCGTTTATTCTCCCTCGGCTCTCGGT
ATTTCACATGAAAACTATAACCTAGACTACACGGGCAACCTTAACCCCAGAGTATAC
TTATATACCAAAGGGATGGGTCCTCAAAAATCACACAAGCAACGACGCCATGAAGT
GTTCGACGTTTTCTTTTTGGTTTGTTTGTAAAATCATTTTCTTTTTCTTTTCTTTCAACA
TCCAAACGTCGATCGCAAACCCTAGAGAGAACTTTCTTAAGTGCTTCTCGCAGTACA
TCCCTAATAACGCTACCAACCTTAAGCTGGTGTACACCCAGAACAACCCTCTTTACA
TGTCTGTTCTAAACAGCACCATCCACAATCTTAGATTCACATCAGACACCACTCCCA
AGCCGCTCGTCATCGTGACCCCGAGTCATGTGTCCCATATCCAAGGCACTATCCTGT
GCTCTAAAAAGGTCGGTCTGCAGATTCGGACTCGCTCCGGTGGACATGATTCGGAGG
GCATGTCCTACATTAGCCAGGTCCCCTTTGTGATCGTGGACCTGAGGAACATGCGGT
CTATTAAGATTGATGTGCACTCACAGACCGCTTGGGTCGAGGCTGGTGCGACATTGG
GTGAGGTGTACTACTGGGTGAACGAGAAGAACGAGAACCTGAGCCTCGCCGCTGGC
TACTGTCCCACCGTTTGTGCCGGTGGACACTTCGGCGGAGGCGGATACGGTCCACTT
ATGCGAAACTACGGGCTCGCAGCTGATAATATCATCGACGCACACCTTGTTAACGTT
CACGGCAAGGTGCTGGACCGAAAAAGCATGGGTGAGGACCTATTTTGGGCCTTGCG
AGGCGGTGGTGCCGAATCCTTCGGAATTATCGTGGCCTGGAAGATCCGACTGGTCGC
TGTGCCAAAGTCCACTATGTTCTCCGTCAAGAAAATTATGGAGATCCACGAACTCGT
AAAGCTCGTCAATAAGTGGCAGAACATCGCCTACAAGTATGACAAGGATCTGCTGC
TCATGACTCACTTCATCACGCGAAACATTACAGACAACCAGGGAAAGAACAAGACC
GCTATCCATACCTACTTCTCCTCTGTCTTCCTTGGGGGTGTCGATTCCCTCGTTGATCT
CATGAACAAATCTTTTCCAGAGCTCGGAATCAAGAAGACCGACTGCCGACAGCTCT
CTTGGATCGACACCATTATTTTCTACTCAGGAGTCGTAAACTACGATACTGACAACT
TTAACAAGGAGATTCTGTTAGATCGATCGGCCGGCCAGAACGGTGCCTTCAAGATCA
AGCTCGACTATGTCAAAAAGCCCATTCCTGAATCCGTCTTCGTTCAAATTCTTGAAA
AGTTGTACGAGGAGGATATCGGCGCCGGAATGTACGCGCTGTACCCCTACGGTGGC
ATTATGGACGAGATTTCTGAAAGTGCTATTCCCTTCCCCCACCGTGCTGGCATTCTGT
ATGAGCTGTGGTACATTTGCTCCTGGGAAAAGCAGGAGGACAACGAGAAGCACTTG
AACTGGATACGAAACATTTACAATTTCATGACCCCCTATGTTTCGAAGAACCCTCGA
CTGGCCTACCTGAATTACCGCGACCTCGACATCGGAATTAACGACCCTAAGAACCCC
AATAACTATACTCAGGCCAGAATCTGGGGCGAGAAGTACTTCGGCAAGAACTTTGA
CCGTCTGGTTAAGGTCAAGACCCTCGTGGACCCTAACAACTTCTTCCGAAACGAGCA
GTCTATCCCCCCTCTGCCCCGACACCGGCATTAATAAGAGTAGGCAATTAACAGATA
GTTTGCCGGTGATAATTCTCTTAACCTCCCACACTCCTTTGACATAACGATTTATGTA
ACGAAACTGAAATTTGACCAGATATTGTTGTAAATAGAAAATCTGGCTTGTAGGTGG
CAAAATGCGGCGTCTTTGTTCATCAATTCCCTCTGTGACTACTCGTCATCCCTTTATG
TTCGACTGTCGTATTTCTTATTTTCCATACATATGCAAGTGAGATGCCCGTGTCCGAA
TTCGCTATGGATCCATAGCGCAGGTTGTGCAGTATCATACATACTCGATCAGACAGG
TCGTCTGACCATCATACAAGCTGAACAAGCGCTCCATACTTGCACGCTCTCTATATA
CACAGTTAAATGACATATCCATAGTCTAACCTCTAACAGTTAATCTTCTGGTAAGCC
TCCCAGCCAGCCTTCTGGTATCGCTTGGCCTCCTCAATAGGATCTCGGTTCTGGCCGT
ACAGACCTCGGCCGACAATTATGATATCCGTTCCGGTAGACATGACATCCTCAACAG
TTCGGTACTGCTGTCCGAGAGCATCTCCCTTGTCGTCAAGACCCACCCCGGGGGTCA
GAATAAGCCAGTCCTCAGAGTCGCCCTTAGGTCGGTTCTGGGCAATGAAGCCAACC
ACAAACTCGGGGTCGGATCGGGCAAGCTCAATGGTCTGCTTGGAGTACTCGCCAGT
GGCCAGAGAGCCCTTGCAAGACAGCTCGGCCAGCATGAGCAGACCTCTGGCCAGCT
TCTCGTTGGGAGAGGGGACCAGGAACTCCTTGTACTGGGAGTTCTCGTAGTCAGAGA
CATCCTCCTTCTTCTGTTCAGAGACAGTTTCCTCGGCACCAGCTCGCAGGCCAGCAA
TGATTCCGGTTCCGGGTACACCGTGGGCGTTGGTGATATCGGACCACTCGGCGATTC
GGTAGACACCGTTCTTGTACTGGTGCTTGACAGTGTTGCCAATATCTGCGAACTTTCT
GTCCTCGAACAGGAAGAAACCGTGCTTAAGAGCAAGTTCCTTGAGGGGGAGCACAG
TTCCGGCGTAGGTGAAGTCGTCAATGATGTCGATATGGGTCTTGATCATGCACACAT
AAGGTCCGACCTTATCGGCAAGCTCAATGAGCTCCTTGGTGGTGGTAACATCCAGAG
AAGCACACAGGTTGGTTTTCTTGGCTGCCACGAGCTTGAGCACTCGGGCGGCAAAG
GCGGACTTGTGGACGTTAGCTCGCGCTTCGTAGGAGGGCATTTTGGTGGTGAAGAGG
AGACTGAAATAAATTTAGTCTGCACAACTTTTTATCGGAACCTTATCTGGGGCAGTG
AAGTATATGTTATGGTAATAGTTACGAGTTAGTTGAACTTATAGATAGACTGGACTA
TACGGCTATCGGTCCAAATTAGAAATTAATTAAATGGTTCTCGAGGATCCAGTCTAC
ACTGATTAATTTTCGGGCCAATAATTTAAAAAAATCGTGTTATATAATATTATATGT
ATTATATATATACATATGATGATACTGACAGTCATGTCCCATTGCTAAATAGACAGA
CTCCATCTGCCGCCTCCAACTGATGTTCTCAATATTTAAGGGGTCATCTCGCATTGTT
TAATAATAAACAGACTCCATCTACCGCCTCCAAATGATGTTCTCAAAATATATTGTA
TGAACTTATTTTTATTACTTAGTATTATTAGACAACTTACTTGCTTTATGAAAAACAC
TTCCTATTTAGGAAACAATTTATAATGGCAGTTCGTTCATTTAACAATTTATGTAGAA
TAAATGTTATAAATGCGTATGGGAAATCTTAAATATGGATAGCATAAATGATATCTG
CATTGCCTAATTCGAAATCAACAGCAACGAAAAAAATCCCTTGTACAACATAAATA
GTCATCGAGAAATATCAACTATCAAAGAACAGCTATTCACACGTTACTATTGAGATT
ATTATTGGACGAGAATCACACACTCAACTGTCTTTCTCTCTTCTAGAAATACAGGTA
CAAGTATGTACTATTCTCATTGTTCATACTTCTAGTCATTTCATCCCACATATTCCTTG
GATTTCTCTCCAATGAATGACATTCTATCTTGCAAATTCAACAATTATAATAAGATAT
ACCAAAGTAGCGGTATAGTGGCAATCAAAAAGCTTCTCTGGTGTGCTTCTCGTATTT
ATTTTTATTCTAATGATCCATTAAAGGTATATATTTATTTCTTGTTATATAATCCTTTT
GTTTATTACATGGGCTGGATACATAAAGGTATTTTGATTTAATTTTTTGCTTAAATTC
AATCCCCCCTCGTTCAGTGTCAACTGTAATGGTAGGAAATTACCATACTTTTGAAGA
AGCAAAAAAAATGAAAGAAATAAAAAATCGTATTTCCAGGTTAGACGTTCCGCAGA
ATCTAGAATGCGGTATGCGGTACATTGTTCTTCGAACGTAAAAGTTGCGCTCCCTGA
GATATTGTACATTTTTGCTTTTACAAGTACAAGTACATCGTACAACTATGTACTACTG
TTGATGCATCCACAACAGTTTGTTTTGTTTTTTTTTGTTTTTTATTTTTCTAATGATTC
ATTACCGCTATGTATACCTACTTGTACTTGTAGTAAGCCGGGTTATTGGCGTTCAATT
AATCATAGACTTATGAATCTGCACGGTGTGCGCTGCGAGTTACTTTTAGCTTATGCA
TGCTACTTGGGTGTAATATTGGGATCAGGTCAGGCGGAATGGCACTTCCTTATTCCA
GATGCGTGCGGATTATGCATGACTGGCCGTCGTTTTACAACGTCGTGACTGGGAAAA
CCCTGGCGCTTGTGGTGGATCACAGTTATTGGAGACTTCCTTGGCCGTACGATGGGT
TTTGTTTCAGGTATCCCAACCTGGACGTCTCGTATAATGCGACAAACAACAGATAAA
ACGAAAGGCCCAGTCTTTCGACTGAGCCTTTCGTTTTATTTGGATCCACTAGTTCTAG
AGCGGCGGGGGAAAGCCACGTTGTGTCTCAAAATCTCTGATGTTACATTGCACAAG
ATAAAAATATATCATCATGAACAATAAAACTGTCTGCTTACATAAACAGTAATACAA
GGGGTGTTATGAGCCATATTCAACGGGAAACGTCTTGCTCGAGGCCGCGATTAAATT
CCAACATGGATGCTGATTTATATGGGTATAAATGGGCTCGCGATAATGTCGGGCAAT
CAGGTGCGACAATCTATCGATTGTATGGGAAGCCCGATGCGCCAGAGTTGTTTCTGA
AACATGGCAAAGGTAGCGTTGCCAATGATGTTACAGATGAGATGGTCAGACTAAAC
TGGCTGACGGAATTTATGCCTCTTCCGACCATCAAGCATTTTATCCGTACTCCTGATG
ATGCATGGTTACTCACCACTGCGATCCCCGGGAAAACAGCATTCCAGGTATTAGAAG
AATATCCTGATTCAGGTGAAAATATTGTTGATGCGCTGGCAGTGTTCCTGCGCCGGT
TGCATTCGATTCCTGTTTGTAATTGTCCTTTTAACAGCGATCGCGTATTTCGTCTCGC
TCAGGCGCAATCACGAATGAATAACGGTTTGGTTGATGCGAGTGATTTTGATGACGA
GCGTAATGGCTGGCCTGTTGAACAAGTCTGGAAAGAAATGCATAAGCTTTTGCCATT
CTCACCGGATTCAGTCGTCACTCATGGTGATTTCTCACTTGATAACCTTATTTTTGAC
GAGGGGAAATTAATAGGTTGTATTGATGTTGGACGAGTCGGAATCGCAGACCGATA
CCAGGATCTTGCCATCCTATGGAACTGCCTCGGTGAGTTTTCTCCTTCATTACAGAAA
CGGCTTTTTCAAAAATATGGTATTGATAATCCTGATATGAATAAATTGCAGTTTCATT
TGATGCTCGATGAGTTTTTCTAATCAGAATTTGTTAATTGGTTGTAACACTGGCAGA
GCGGACGATTTGAAGCCCCCTACCCATTTACGCTGACTTGACGGGACGGCGGCTTTG
TTGAATAGAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGCGGTGGTT
TGTTTGCCGGATCAAGAGCTACCAACTCTTTTTCCGAAGGTAACTGGCTTCAGCAGA
GCGCAGATACCAAATACTGTTCTTCTAGTGTAGCCGTAGTTAGGCCACCACTTCAAG
AACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCGGAT
AAGGCGCAGCGGTCGGGCTGAACGGGGGGTTCGTGCACACAGCCCAGCTTGGAGCG
AACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGG
AGAGCGCACGAGGGAGCTTCCAGGGGGAAACGCCTGGTATCTTTATAGTCCTGTCG
GGTTTCGCCACCTCTGACTTGAGCGTCGATTTTTGTGATGCTCGTCAGGGGGGCGGA
GCCTATGGAAAAACGCCAGCAACGCGGCCTTTTTACGGTTCCTGGCCTTTTGCTGGC
CTTTTGCTCACATGTTCTTTCCAGCGACATATGCCATACGCCGAACTGAACTCCACCT
AATGTCTTGGAGATTGTCGGATAGCGCGGAATAGGATTGTGCCCTCTGAGCGGATAA
CAATTTCACACAGGAAACAGCTATGACCATGACACATGCATCCACCATCGCAGACTT
ATCATCACTGAGCCTCCACCTAGCCT
pLD135
(SEQ ID NO: 176)
AGGCTAGGTGGAGGCTCAGTGATGATAAGTCTGCGATGGTGGATGCATGTGTCATG
GTCATAGCTGTTTCCTGTGTGAAATTGTTATCCGCTCAGAGGGCACAATCCTATTCCG
CGCTATCCGACAATCTCCAAGACATTAGGTGGAGTTCAGTTCGGCGTATGGCATATG
TCGCTGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAAAA
TCGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCC
GTTCAGCCCGACCGCTGCGCCTTATCCGGTAACTATCGTCTTGAGTCCAACCCGGTA
AGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAG
AAGAACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTT
CTACGGGGTCTGACGCTCTATTCAACAAAGCCGCCGTCCCGTCAAGTCAGCGTAAAT
GGGTAGGGGGCTTCAAATCGTCCTCGTGATACCAATTCGGAGCCTGCTTTTTTGTAC
AAACTTGTTGATAATGGCAATTCAAGGATCTTCACCTAGATCCTTTTAAATTAAAAA
TGAAGTTTTAAATCAATCTAAAGTATATATGAGTAAACTTGGTCTGACAGTTACCAA
TGCTTAATCAGTGAGGCACCTATCTCAGCGATCTGTCTATTTCGTTCATCCATAGTTG
CCTGACTCCCCGTCGTGTAGATAACTACGATACGGGAGGGCTTACCATCTGGCCCCA
GTGCTGCAATGATACCGCGAGAGCCACGCTCACCGGCTCCAGATTTATCAGCAATA
AACCAGCCAGCCGGAAGGGCCGAGCGCAGAAGTGGTCCTGCAACTTTATCCGCCTC
CATCCAGTCTATTAATTGTTGCCGGGAAGCTAGAGTAAGTAGTTCGCCAGTTAATAG
TTTGCGCAACGTTGTTGCCATTGCTACAGGCATCGTGGTGTCACGCTCGTCGTTTGGT
ATGGCTTCATTCAGCTCCGGTTCCCAACGATCAAGGCGAGTTACATGATCCCCCATG
TTGTGCAAAAAAGCGGTTAGCTCCTTCGGTCCTCCGATCGTTGTCAGAAGTAAGTTG
GCCGCAGTGTTATCACTCATGGTTATGGCAGCACTGCATAATTCTCTTACTGTCATGC
CATCCGTAAGATGCTTTTCTGTGACTGGTGAGTACTCAACCAAGTCATTCTGAGAAT
AGTGTATGCGGCGACCGAGTTGCTCTTGCCCGGCGTCAATACGGGATAATACCGCGC
CACATAGCAGAACTTTAAAAGTGCTCATCATTGGAAAACGTTCTTCGGGGCGAAAA
CTCTCAAGGATCTTACCGCTGTTGAGATCCAGTTCGATGTAACCCACTCGTGCACCC
AACTGATCTTCAGCATCTTTTACTTTCACCAGCGTTTCTGGGTGAGCAAAAACAGGA
AGGCAAAATGCCGCAAAAAAGGGAATAAGGGCGACACGGAAATGTTGAATACTCA
TACTCTTCCTTTTTCAATATTATTGAAGCATTTATCAGGGTTATTGTCTCATGAGCGG
ATACATATTTGAATGTATTTAGAAAAATAAACAAATAGGGGTTCCGCGCACATTTCC
CCGAAAAGTGCCAGATACCTGAAACAAAACCCATCGTACGGCCAAGGAAGTCTCCA
ATAACTGTGATCCACCACAAGCGCCAGGGTTTTCCCAGTCACGACGTTGTAAAACGA
CGGCCAGTCATGCATAATCCGCACGCATCTGGAATAAGGAAGTGCCATTCCGCCTG
ACCTTTAATTAATTGGCAAATTTTACTGTGGCCTTCAGAACGGTAAAAATAGACCAA
TCAGAATTCTGAAAAGCACATCTTGATCTCCTCATTGCGGGGAGTCCAACGGTGGTC
TTATTCCCCCGAATTTCCCGCTCAATCTCGTTCCAGACCGACCCGGACACAGTGCTT
AACGCCGTTCCGAAACTCTACCGCAGATATGCTCCAACGGACTGGGCTGCATAGAT
GTGATCCTCGGCTTGGAGAAATGGATAAAAGCCGGCCAAAAAAAAAGCGGAAAAA
AGCGGAAAAAAAGAGAAAAAAAATCGCAAAATTTGAAAAATAGGGGGAAAAGACG
CAAAAACGCAAGGAGGGGGGAGTATATGACACTGATAAGCAAGCTCACAACGGTTC
CTCTTATTTTTTTCCTCATCTTCTGCCTAGGTTCCCAAAATCCCAGATGCTTCTCTCCA
GTGCCAAAAGTAAGTACCCCACAGGTTTTCGGCCGAAAATTCCACGTGCAGCAACG
TCGTGTGGGGTGTTAAAATGTGGGGGCGGGGAACCAGGACAAGAGGCTCTTGTGGG
AGCCGAATGAGAGCACAAAGCGGGCGGGTGTGATAAGGGCATTTTTGCCCATTTTC
CCTTCTCCTGTCTCTCCGACGGTGATGGCGTTGTGCGTCCTCTATCTATTTCTTTTTAT
TTCTTTTTGTTTTATTTCTCTGACTACCGATTTGGCTTGATTTCCTCAACCCCACACAA
ATAAGCTCGGGCCGAGGAATATATATATACACGGACACAGTCGCCCTGTGGACAAC
ACGTCACTACCTCTACGACGCTATGGATCCATAGCGAATTCGGACACGGGCATCTCA
CTTGCATATGTATGGAAAATAAGAAATACGACAGTCGAACATAAAGGGATGACGAG
TAGTCACAGAGGGAATTGATGAACAAAGACGCCGCATTTTGCCACCTACAAGCCAG
ATTTTCTATTTACAACAATATCTGGTCAAATTTCAGTTTCGTTACATAAATCGTTATG
TCAAAGGAGTGTGGGAGGTTAAGAGAATTATCACCGGCAAACTATCTGTTAATTGCC
TACTCTTATTACAGTTTGGCTCGTCGTCCCCGAGACTTGCCGGCCTTGATAGCGAGG
AGTGTCTTCTCGTCAGCAGGGGCGCTACCGGAGGCTCGTTGCTGTCTGAAGTAGGTT
CGGGCCTCGTCCTGTCGGGTGGCCTCAGCGCCGGTGGCGATAGCGGCCCGAAGGTG
CTCGGCTCCGTAGCCGAAGGCATCAACCAGATCAAGTGCGTGTGGTCGGATCTTAAC
GAGAAGACGATTAATGTAAGTGCCGACCGTTCGGCCTCTCTGCATCGAGAGCCTACC
GTTCATCAGATACCAGCTGAGGTGCTTCTCGATAAGGGACAGACCAAAAAGGTCAC
GCAATCGGGTCAACACTTCCTTAGTGCCAGCGTCGTCCACCTTGGCCAAAGCCTCAG
TAAAGGCCTCCCACTGTAAAAGCTCAGCGTGGGCCTGGGCAGCCTCGATCAGTTCGT
TTTGATGCTGATTGAACAGTGCTGCCGCCTGGTGCTGGGGGAGCTTGCCCGCGCCTT
TGAGAGCAGCGCCAACCTCTGCAACCATGGACTGGACTCTGTCGGTCAGCAGAGTT
CGCTGACCTTCTTCGTCTCGGAGGGCAAGAGCAGATTTCTGGACAGATCCACTGTCG
GCCACGAACTGAGCGACCTGTCGCAGTCCGGTTCGGTGTAGCGCGACTCCGGCAGC
CTGGTCCACGACGTACCGCGCGAGCACGCCGAAGTTGGCACCTCGGAACTCCTTAG
CATAGTCAGCGAGCAGCCTCTTGGCCACCAACTGAAGAAGCACGGTGTTGTCTCCTT
CGAAGGTCACGTAGACGTCGAGATCGGCCCGGAGAGAAGCGAATCGGTTCTCAATC
AGAAAACCTGCACCCCCACACGCTTCTCGGCACTCTTGTAGGGTATCGAGAGCATGC
CAGGTAGAAAGGGGCTTCAGAGCAGCAGCCAGTGTCTCAAGGTCCTGACGGTCGGC
GTCAGTATCATGAGCACCCGAGAACACGTCGTCGAATTTCTGCAGCAGTTGCTCATG
GGCGAACGATGCGGCGTAGGTGGTGGCGAGTCGGGTAAAGAGGCGCCGCTGGTGTC
GCTGGTAATCCAGGAGCACCTCCTCCTCTGTGGGCGAAGTGGCGTTGAACTGTCTTC
GCTCAGCGGCGTAGTGGATGGCGGATTGCAGAGCAACCTTCGATGCAGCCACTGCA
GCACCGTCCAGGCTGACTCGGCCCTGGACCAGCGTACCTAGCATTGTAAAGAATCTT
CGCCCCGGAGATTCGATGGTTGAGCTGTAGGTGCCATCGACGGCAACATCGCCGTA
ACGGTTAAGCAGGTTGGTTCGGGGAATGCGAACATTGGTGAAATGGAGACGTCCGT
TATCAATTCCGTTTAGTCCCCCCTTAATACCGTCGTCCTCTCCACCAATACCAGGGAG
AAAGTCTCCGGTGGCGGGATCTCTCAGATCGACGTAGAAGGCGTGCACTCCATGGTT
AACCTTTCGAGTAATCAGTTGGGCAAACACGACAGCGGCCAAACCGTCGTTAGCGG
CGTTTCCAATGTAGTCCTTCCAGGCGGCTCGGAACGGGGTGTCAATAACGAACTCCT
GGGTTTCCTCATCATAGGTGGCCGTTGTAGCAATGGAAGCGACATCGGAGCCATGGC
CAGTCTCAGTCATGGCAAAGCAACCAGGGATTTCCAGAGACATGATGCCTGGGAGC
CACTTGTCGTGGTGCTCCCTAGTTCCCAAGTGCATAACAGCGGAGCCGAACAGTCCC
CACTGGACGCCGGCCTTGATCTGGAGGGAGGGGTCCGCCGTGACAAGCTCCTCAAA
ACCAGCGATGTTTCCGCCGTGGTCGTCACTACCACCCAGTCGAGAAGGAAAGGCTC
GGTGAACTGCGTTGTTATCGACCAAGTACTTGAGCTGGCCAAAGACGCGAGAGCGG
TGCTCTGTATGAGTCAGACCCTCCACCTTCTGAACTACCTCTCGTCCCGCAAGGTCCC
GGGCGTGGAGACGGATATCAGCCCATCGGCCCAGCAGCTGCTCTCCCAGCGCAGCC
ACATCTACTCGGGGCTCGACGGCCACCTTAGCACCGTCTGCGGCGGCCGTAGTTGAG
CCAGGGGATGCGGGGGATGAGGCTCTGTCAACTACTTCGGTCATGGCGTCGTTGCTT
GTGTGATTTTTGAGGACCCATCCCTTTGGTATATAAGTATACTCTGGGGTTAAGGTTG
CCCGTGTAGTCTAGGTTATAGTTTTCATGTGAAATACCGAGAGCCGAGGGAGAATAA
ACGGGGGTATTTGGACTTGTTTTTTTCGCGGAAAAGCGTCGAATCAACCCTGCGGGC
CTTGCACCATGTCCACGACGTGTTTCTCGCCCCAATTCGCCCCTTGCACGTCAAAATT
AGGCCTCCATCTAGACCCCTCCATAACATGTGACTGTGGGGAAAAGTATAAGGGAA
ACCATGCAACCATAGACGACGTGAAAGACGGGGAGGAACCAATGGAGGCCAAAGA
AATGGGGTAGCAACAGTCCAGGAGACAGACAAGGAGACAAGGAGAGGGCGCCCGA
AAGATCGGAAAAACAAACATGTCCAATTGGGGCAGTGACGGAAACGACACGGACA
CTTCAGTACAATGGACCGACCATCTCCAAGCCAGGGTTATTCCGGTATCACCTTGGC
CGTAACCTCCCGCTGGTACCTGATATTGTACACGTTCACATTCAATATACTTTCAGCT
ACAATAAGAGAGGCTGTTTGTCGGGCATGTGTGTCCGTCGTATGGGGTGATGTCCGA
GGGCGAAATTCGCTACAAGCTTAACTCTGGCGCTTGTCCAGTATGAATAGACAAGTC
AAGACCAGTGGTGCCATGATTGACAGGGAGGTACAAGACTTCGATACTCAAGCATT
ACTCGGACTTGTGGCGATTGAACAGACGGGCGATCGCTTCTCCCCCGTATTGCCCCG
TTTCTACGAGTGCCGTACGAGTACGAGTACAGGAGGTACAGTACATTCCGATACTTG
CACTCCCACCATTGCTGTATATCTCTGCTACTTGTGGTATCAAATCCGCTACTCGTAG
TGGTTTTTCCACTTGTACAAATTCTATCCCCCATGACCACCAACCCTTCTCCCGATCG
CCAGTCTCACTTGGCGGAATCGTCGACGATCATCCTCGAGATGCGGCCGCGTCGACA
TAACTTCGTATAGCATACATTATACGAAGTTATTTTCTAATTTGGACCGATAGCCGTA
TAGTCCAGTCTATCTATAAGTTCAACTAACTCGTAACTATTACCATAACATATACTTC
ACTGCCCCAGATAAGGTTCCGATAAAAAGTTGTGCAGACTAAATTTATTTCAGTCTC
CTCTTCACCACCAAAATGCCCTCCTACGAAGCGCGAGCTAACGTCCACAAGTCCGCC
TTTGCCGCCCGAGTGCTCAAGCTCGTGGCAGCCAAGAAAACCAACCTGTGTGCTTCT
CTGGATGTTACCACCACCAAGGAGCTCATTGAGCTTGCCGATAAGGTCGGACCTTAT
GTGTGCATGATCAAGACCCATATCGACATCATTGACGACTTCACCTACGCCGGAACT
GTGCTCCCCCTCAAGGAACTTGCTCTTAAGCACGGTTTCTTCCTGTTCGAGGACAGA
AAGTTCGCAGATATTGGCAACACTGTCAAGCACCAGTACAAGAACGGTGTCTACCG
AATCGCCGAGTGGTCCGATATCACCAACGCCCACGGTGTACCCGGAACCGGAATCA
TTGCTGGCCTGCGAGCTGGTGCCGAGGAAACTGTCTCTGAACAGAAGAAGGAGGAT
GTCTCTGACTACGAGAACTCCCAGTACAAGGAGTTCCTGGTCCCCTCTCCCAACGAG
AAGCTGGCCAGAGGTCTGCTCATGCTGGCCGAGCTGTCTTGCAAGGGCTCTCTGGCC
ACTGGCGAGTACTCCAAGCAGACCATTGAGCTTGCCCGATCCGACCCCGAGTTTGTG
GTTGGCTTCATTGCCCAGAACCGACCTAAGGGCGACTCTGAGGACTGGCTTATTCTG
ACCCCCGGGGTGGGTCTTGACGACAAGGGAGATGCTCTCGGACAGCAGTACCGAAC
TGTTGAGGATGTCATGTCTACCGGAACGGATATCATAATTGTCGGCCGAGGTCTGTA
CGGCCAGAACCGAGATCCTATTGAGGAGGCCAAGCGATACCAGAAGGCTGGCTGGG
AGGCTTACCAGAAGATTAACTGTTAGAGGTTAGACTATGGATATGTCATTTAACTGT
GTATATAGAGAGCGTGCAAGTATGGAGCGCTTGTTCAGCTTGTATGATGGTCAGACG
ACCTGTCTGATCGAGTATGTATGATACTGCACAACCTGATAACTTCGTATAGCATAC
ATTATACGAAGTTATCTCGAGGCGGCCGCATGGAGCGTGTGTTCTGAGTCGATGTTT
TCTATGGAGTTGTGAGTGTTAGTAGACATGATGGGTTTATATATGATGAATGAATAG
ATGTGATTTTGATTTGCACGATGGAATTGAGAACTTTGTAAACGTACATGGGAATGT
ATGAATGTGGGGGTTTTGTGACTGGATAACTGACGGTCAGTGGACGCCGTTGTTCAA
ATATCCAAGAGATGCGAGAAACTTTGGGTCAAGTGAACATGTCCTCTCTGTTCAAGT
AAACCATCAACTATGGGTAGTATATTTAGTAAGGACAAGAGTTGAGATTCTTTGGAG
TCCTAGAAACGTATTTTCGCGTTCCAAGATCAAATTAGTAGAGTAATACGGGCACGG
GAATCCATTCATAGTCTCAATTTTCCCATAGGTGTGCTACAAGGTGTTGAGATGTGG
TACAGTACCACCATGATTCGAGGTAAAGAGCCCAGAAGTCATTGATGAGGTCAAGA
AATACACAGATCTACAGCTCAATACAATGAATATCTTCTTTCATATTCTTCAGGTGA
CACCAAGGGTGTCTATTTTCCCCAGAAATGCGTGAAAAGGCGCGTGTGTAGCGTGG
AGTATGGGTTCGGTTGGCGTATCCTTCATATATCGACGAAATAGTAGGGCAAGAGAT
GACAAAAAGTATCTATATGTAGACAGCGTAGAATATGGATTTGATTGGTATAAATTC
ATTTATTGCGTGTCTCACAAATACTCTCGATAAGTTGGGGTTAAACTGGAGATGGAA
CAATGTCGATATCTCGACATATTTTGATATTTGTTAATTAA
pLD137
(SEQ ID NO: 177)
AGGCTAGGTGGAGGCTCAGTGATGATAAGTCTGCGATGGTGGATGCATGTGTCATG
GTCATAGCTGTTTCCTGTGTGAAATTGTTATCCGCTCAGAGGGCACAATCCTATTCCG
CGCTATCCGACAATCTCCAAGACATTAGGTGGAGTTCAGTTCGGCGTATGGCATATG
TCGCTGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAAAA
TCGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCC
GTTCAGCCCGACCGCTGCGCCTTATCCGGTAACTATCGTCTTGAGTCCAACCCGGTA
AGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAG
AAGAACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTT
CTACGGGGTCTGACGCTCTATTCAACAAAGCCGCCGTCCCGTCAAGTCAGCGTAAAT
GGGTAGGGGGCTTCAAATCGTCCGCTCTGCCAGTGTTACAACCAATTAACAAATTCT
GATTAGAAAAACTCATCGAGCATCAAATGAAACTGCAATTTATTCATATCAGGATTA
TCAATACCATATTTTTGAAAAAGCCGTTTCTGTAATGAAGGAGAAAACTCACCGAGG
CAGTTCCATAGGATGGCAAGATCCTGGTATCGGTCTGCGATTCCGACTCGTCCAACA
TCAATACAACCTATTAATTTCCCCTCGTCAAAAATAAGGTTATCAAGTGAGAAATCA
CCATGAGTGACGACTGAATCCGGTGAGAATGGCAAAAGCTTATGCATTTCTTTCCAG
ACTTGTTCAACAGGCCAGCCATTACGCTCGTCATCAAAATCACTCGCATCAACCAAA
CCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATC
AACAATATTTTCACCTGAATCAGGATATTCTTCTAATACCTGGAATGCTGTTTTCCCG
GGGATCGCAGTGGTGAGTAACCATGCATCATCAGGAGTACGGATAAAATGCTTGAT
GGTCGGAAGAGGCATAAATTCCGTCAGCCAGTTTAGTCTGACCATCTCATCTGTAAC
ATCATTGGCAACGCTACCTTTGCCATGTTTCAGAAACAACTCTGGCGCATCGGGCTT
CCCATACAATCGATAGATTGTCGCACCTGATTGCCCGACATTATCGCGAGCCCATTT
ATACCCATATAAATCAGCATCCATGTTGGAATTTAATCGCGGCCTCGAGCAAGACGT
TTCCCGTTGAATATGGCTCATAACACCCCTTGTATTACTGTTTATGTAAGCAGACAGT
TTTATTGTTCATGATGATATATTTTTATCTTGTGCAATGTAACATCAGAGATTTTGAG
ACACAACGTGGCTTTCCCCCGCCGCTCTAGAACTAGTGGATCCAAATAAAACGAAA
GGCTCAGTCGAAAGACTGGGCCTTTCGTTTTATCTGTTGTTTGTCGCATTATACGAGA
CGTCCAGGTTGGGATACCTGAAACAAAACCCATCGTACGGCCAAGGAAGTCTCCAA
TAACTGTGATCCACCACAAGCGCCAGGGTTTTCCCAGTCACGACGTTGTAAAACGAC
GGCCAGTCATGCATAATCCGCACGCATCTGGAATAAGGAAGTGCCATTCCGCCTGA
CCTGATCCCAATATTACACCCAAGTAGCATGCATAAGCTAAAAGTAACTCGCAGCG
CACACCGTGCAGATTCATAAGTCTATGATTAATTGAACGCCAATAACCCGGCTTACT
ACAAGTACAAGTAGGTATACATAGCGGTAATGAATCATTAGAAAAATAAAAAACAA
AAAAAAACAAAACAAACTGTTGTGGATGCATCAACAGTAGTACATAGTTGTACGAT
GTACTTGTACTTGTAAAAGCAAAAATGTACAATATCTCAGGGAGCGCAACTTTTACG
TTCGAAGAACAATGTACCGCATACCGCATTCTAGATTCTGCGGAACGTCTAACCTGG
AAATACGATTTTTTATTTCTTTCATTTTTTTTGCTTCTTCAAAAGTATGGTAATTTCCT
ACCATTACAGTTGACACTGAACGAGGGGGGATTGAATTTAAGCAAAAAATTAAATC
AAAATACCTTTATGTATCCAGCCCATGTAATAAACAAAAGGATTATATAACAAGAA
ATAAATATATACCTTTAATGGATCATTAGAATAAAAATAAATACGAGAAGCACACC
AGAGAAGCTTTTTGATTGCCACTATACCGCTACTTTGGTATATCTTATTATAATTGTT
GAATTTGCAAGATAGAATGTCATTCATTGGAGAGAAATCCAAGGAATATGTGGGAT
GAAATGACTAGAAGTATGAACAATGAGAATAGTACATACTTGTACCTGTATTTCTAG
AAGAGAGAAAGACAGTTGAGTGTGTGATTCTCGTCCAATAATAATCTCAATAGTAA
CGTGTGAATAGCTGTTCTTTGATAGTTGATATTTCTCGATGACTATTTATGTTGTACA
AGGGATTTTTTTCGTTGCTGTTGATTTCGAATTAGGCAATGCAGATATCATTTATGCT
ATCCATATTTAAGATTTCCCATACGCATTTATAACATTTATTCTACATAAATTGTTAA
ATGAACGAACTGCCATTATAAATTGTTTCCTAAATAGGAAGTGTTTTTCATAAAGCA
AGTAAGTTGTCTAATAATACTAAGTAATAAAAATAAGTTCATACAATATATTTTGAG
AACATCATTTGGAGGCGGTAGATGGAGTCTGTTTATTATTAAACAATGCGAGATGAC
CCCTTAAATATTGAGAACATCAGTTGGAGGCGGCAGATGGAGTCTGTCTATTTAGCA
ATGGGACATGACTGTCAGTATCATCATATGTATATATATAATACATATAATATTATA
TAACACGATTTTTTTAAATTATTGGCCCGAAAATTAATCAGTGTAGACTGGATCCTC
GAGAACCATTTAATTAAGATCAGAGACCGGATGGTCTCAAGCGTCGACACCATATC
ATATAAAACTAACAATGCATTGAATTAATACGAAGACTACCCGTTGCTATCTCCACA
CCGTTATCTCCACGGTCCAAAGGCTGCTCAATGTGCTGCATACGTAACGTGGGGTGC
AACCTTGAGCACATAGTACTTTCCGAAAACCGGCGATAATTAAGTGTGCACTCCAAC
TTTTCACACTGAGCATAAAATGTGGAGAAGAAATCACACTAAAAAGTCAGGTAGAC
TGGAAAATGCGCCATGAAATAATATCTCTTGCTACAGTAATGCCCAGCATCGAGGG
GTATTGTGTCACCAACACTATAGTGGCAGCTGAAGCGCTCGTGATTGTAGTATGAGT
CTTTATTGGTGATGGGAAGAGTTCACTCAATATTCTCGTTACTGCCAAAACACCACG
GTAATCGGCCAGACACCATGGATGTAGATCACCAAGCCTGTGAATGTTATTCGAGCT
AAAATGCACATGGTTGGTGAAAGGAGTAGTTGCTGTCGAATTCCGTCGTCGCCTGAG
TCATCATTTATTTACCAGTTGGCCACAAACCCTTGACGATCTCGTATGTCCCCTCCGA
CATACTCCCGGCCGGCTGGGGTACGTTCGATAGCGCTATCGGCATCGACAAGGTTTG
GGTCCCTAGCCGATACCGCACTACCTGAGTCACAATCTTCGGAGGTTTAGTCTTCCA
CATAGCACGGGCAAAAGTGCGTATATATACAAGAGCGTTTGCCAGCCACAGATTTTC
ACTCCACACACCACATCACACATACAACCACACACATCCACAATGGAACCCGAAAC
TAAGAAGACCAAGACTGACTCCAAGAAGATTGTTCTTCTCGGCGGCGACTTCTGTGG
CCCCGAGGTGATTGCCGAGGCCGTCAAGGTGCTCAAGTCTGTTGCTGAGGCCTCCGG
CACCGAGTTTGTGTTTGAGGACCGACTCATTGGAGGAGCTGCCATTGAGAAGGAGG
GCGAGCCCATCACCGACGCTACTCTCGACATCTGCCGAAAGGCTGACTCTATTATGC
TCGGTGCTGTCGGAGGCGCTGCCAACACCGTATGGACCACTCCCGACGGACGAACC
GACGTGCGACCCGAGCAGGGTCTTCTCAAGCTGCGAAAGGACCTGAACCTGTACGC
CAACCTGCGACCCTGCCAGCTGCTGTCGCCCAAGCTCGCCGATCTCTCCCCCATCCG
AAACGTTGAGGGCACCGACTTCATCATTGTCCGAGAGCTCGTCGGAGGTATCTACTT
TGGAGAGCGAAAGGAGGATGACGGATCTGGCGTCGCTTCCGACACTGAGACTTACT
CCGTTCCTGAGGTTGAGCGAATTGCCCGAATGGCCGCCTTCCTGGCCCTTCAGCACA
ACCCCCCTCTTCCCGTGTGGTCCCTTGACAAGGCCAACGTGCTGGCCTCCTCTCGACT
TTGGCGAAAGACCGTCACCCGAGTCCTCAAGGACGAATTCCCCCAGCTGGAGCTCA
ACCACCAGCTGATCGACTCGGCCGCCATGATCCTCATCAAGCAGCCCTCCAAGATGA
ATGGTATCATCATCACCACCAACATGTTTGGCGATATCATCTCCGACGAGGCCTCCG
TCATCCCCGGTTCTCTGGGTCTGCTGCCCTCCGCCTCTCTGGCTTCTCTGCCCGACAC
CAACGAGGCGTTCGGTCTGTACGAGCCCTGTCACGGATCTGCCCCCGATCTCGGCAA
GCAGAAGGTCAACCCCATTGCCACCATTCTGTCTGCCGCCATGATGCTCAAGTTCTC
TCTTAACATGAAGCCCGCCGGTGACGCTGTTGAGGCTGCCGTCAAGGAGTCCGTCGA
GGCTGGTATCACTACCGCCGATATCGGAGGCTCCTCCTCCACCTCCGAGGTCGGAGA
CTTTGTTGCCAACAAGGTCAAGGAGCTGCTCAAGAAGGAGTAAGTCGTTTCTACGAC
GCATTGATGGAAGGAGCAAACTGACGCGCCTGCGGGTTGGTCTACCGGCAGGGTCC
GCTAGTGTATAAGACTCTATAAAAAGGGCCCAGCCCTGCTAATGAAATGATGATTTA
TAATTTACCGGTGTAGCAACCTTGACTAGAAGAAGCAGATTGGGTGTGTTTGTAGTG
GAGGACAGTGGTACGTTTTAATTAA
pLD138
(SEQ ID NO: 178)
AGGCTAGGTGGAGGCTCAGTGATGATAAGTCTGCGATGGTGGATGCATGTGTCATG
GTCATAGCTGTTTCCTGTGTGAAATTGTTATCCGCTCAGAGGGCACAATCCTATTCCG
CGCTATCCGACAATCTCCAAGACATTAGGTGGAGTTCAGTTCGGCGTATGGCATATG
TCGCTGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAAAA
TCGACGCTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCC
GTTCAGCCCGACCGCTGCGCCTTATCCGGTAACTATCGTCTTGAGTCCAACCCGGTA
AGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAG
AAGAACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGATCTTTT
CTACGGGGTCTGACGCTCTATTCAACAAAGCCGCCGTCCCGTCAAGTCAGCGTAAAT
GGGTAGGGGGCTTCAAATCGTCCGCTCTGCCAGTGTTACAACCAATTAACAAATTCT
GATTAGAAAAACTCATCGAGCATCAAATGAAACTGCAATTTATTCATATCAGGATTA
TCAATACCATATTTTTGAAAAAGCCGTTTCTGTAATGAAGGAGAAAACTCACCGAGG
CAGTTCCATAGGATGGCAAGATCCTGGTATCGGTCTGCGATTCCGACTCGTCCAACA
TCAATACAACCTATTAATTTCCCCTCGTCAAAAATAAGGTTATCAAGTGAGAAATCA
CCATGAGTGACGACTGAATCCGGTGAGAATGGCAAAAGCTTATGCATTTCTTTCCAG
ACTTGTTCAACAGGCCAGCCATTACGCTCGTCATCAAAATCACTCGCATCAACCAAA
CCGTTATTCATTCGTGATTGCGCCTGAGCGAGACGAAATACGCGATCGCTGTTAAAA
GGACAATTACAAACAGGAATCGAATGCAACCGGCGCAGGAACACTGCCAGCGCATC
AACAATATTTTCACCTGAATCAGGATATTCTTCTAATACCTGGAATGCTGTTTTCCCG
GGGATCGCAGTGGTGAGTAACCATGCATCATCAGGAGTACGGATAAAATGCTTGAT
GGTCGGAAGAGGCATAAATTCCGTCAGCCAGTTTAGTCTGACCATCTCATCTGTAAC
ATCATTGGCAACGCTACCTTTGCCATGTTTCAGAAACAACTCTGGCGCATCGGGCTT
CCCATACAATCGATAGATTGTCGCACCTGATTGCCCGACATTATCGCGAGCCCATTT
ATACCCATATAAATCAGCATCCATGTTGGAATTTAATCGCGGCCTCGAGCAAGACGT
TTCCCGTTGAATATGGCTCATAACACCCCTTGTATTACTGTTTATGTAAGCAGACAGT
TTTATTGTTCATGATGATATATTTTTATCTTGTGCAATGTAACATCAGAGATTTTGAG
ACACAACGTGGCTTTCCCCCGCCGCTCTAGAACTAGTGGATCCAAATAAAACGAAA
GGCTCAGTCGAAAGACTGGGCCTTTCGTTTTATCTGTTGTTTGTCGCATTATACGAGA
CGTCCAGGTTGGGATACCTGAAACAAAACCCATCGTACGGCCAAGGAAGTCTCCAA
TAACTGTGATCCACCACAAGCGCCAGGGTTTTCCCAGTCACGACGTTGTAAAACGAC
GGCCAGTCATGCATAATCCGCACGCATCTGGAATAAGGAAGTGCCATTCCGCCTGA
CCTGATCCCAATATTACACCCAAGTAGCATGCATAAGCTAAAAGTAACTCGCAGCG
CACACCGTGCAGATTCATAAGTCTATGATTAATTGAACGCCAATAACCCGGCTTACT
ACAAGTACAAGTAGGTATACATAGCGGTAATGAATCATTAGAAAAATAAAAAACAA
AAAAAAACAAAACAAACTGTTGTGGATGCATCAACAGTAGTACATAGTTGTACGAT
GTACTTGTACTTGTAAAAGCAAAAATGTACAATATCTCAGGGAGCGCAACTTTTACG
TTCGAAGAACAATGTACCGCATACCGCATTCTAGATTCTGCGGAACGTCTAACCTGG
AAATACGATTTTTTATTTCTTTCATTTTTTTTGCTTCTTCAAAAGTATGGTAATTTCCT
ACCATTACAGTTGACACTGAACGAGGGGGGATTGAATTTAAGCAAAAAATTAAATC
AAAATACCTTTATGTATCCAGCCCATGTAATAAACAAAAGGATTATATAACAAGAA
ATAAATATATACCTTTAATGGATCATTAGAATAAAAATAAATACGAGAAGCACACC
AGAGAAGCTTTTTGATTGCCACTATACCGCTACTTTGGTATATCTTATTATAATTGTT
GAATTTGCAAGATAGAATGTCATTCATTGGAGAGAAATCCAAGGAATATGTGGGAT
GAAATGACTAGAAGTATGAACAATGAGAATAGTACATACTTGTACCTGTATTTCTAG
AAGAGAGAAAGACAGTTGAGTGTGTGATTCTCGTCCAATAATAATCTCAATAGTAA
CGTGTGAATAGCTGTTCTTTGATAGTTGATATTTCTCGATGACTATTTATGTTGTACA
AGGGATTTTTTTCGTTGCTGTTGATTTCGAATTAGGCAATGCAGATATCATTTATGCT
ATCCATATTTAAGATTTCCCATACGCATTTATAACATTTATTCTACATAAATTGTTAA
ATGAACGAACTGCCATTATAAATTGTTTCCTAAATAGGAAGTGTTTTTCATAAAGCA
AGTAAGTTGTCTAATAATACTAAGTAATAAAAATAAGTTCATACAATATATTTTGAG
AACATCATTTGGAGGCGGTAGATGGAGTCTGTTTATTATTAAACAATGCGAGATGAC
CCCTTAAATATTGAGAACATCAGTTGGAGGCGGCAGATGGAGTCTGTCTATTTAGCA
ATGGGACATGACTGTCAGTATCATCATATGTATATATATAATACATATAATATTATA
TAACACGATTTTTTTAAATTATTGGCCCGAAAATTAATCAGTGTAGACTGGATCCTC
GAGAACCATTTAATTAATTTCTAATTTGGACCGATAGCCGTATAGTCCAGTCTATCT
ATAAGTTCAACTAACTCGTAACTATTACCATAACATATACTTCACTGCCCCAGATAA
GGTTCCGATAAAAAGTTGTGCAGACTAAATTTATTTCAGTCTCCTCTTCACCACCAA
AATGCCCTCCTACGAAGCGCGAGCTAACGTCCACAAGTCCGCCTTTGCCGCCCGAGT
GCTCAAGCTCGTGGCAGCCAAGAAAACCAACCTGTGTGCTTCTCTGGATGTTACCAC
CACCAAGGAGCTCATTGAGCTTGCCGATAAGGTCGGACCTTATGTGTGCATGATCAA
GACCCATATCGACATCATTGACGACTTCACCTACGCCGGAACTGTGCTCCCCCTCAA
GGAACTTGCTCTTAAGCACGGTTTCTTCCTGTTCGAGGACAGAAAGTTCGCAGATAT
TGGCAACACTGTCAAGCACCAGTACAAGAACGGTGTCTACCGAATCGCCGAGTGGT
CCGATATCACCAACGCCCACGGTGTACCCGGAACCGGAATCATTGCTGGCCTGCGA
GCTGGTGCCGAGGAAACTGTCTCTGAACAGAAGAAGGAGGATGTCTCTGACTACGA
GAACTCCCAGTACAAGGAGTTCCTGGTCCCCTCTCCCAACGAGAAGCTGGCCAGAG
GTCTGCTCATGCTGGCCGAGCTGTCTTGCAAGGGCTCTCTGGCCACTGGCGAGTACT
CCAAGCAGACCATTGAGCTTGCCCGATCCGACCCCGAGTTTGTGGTTGGCTTCATTG
CCCAGAACCGACCTAAGGGCGACTCTGAGGACTGGCTTATTCTGACCCCCGGGGTG
GGTCTTGACGACAAGGGAGATGCTCTCGGACAGCAGTACCGAACTGTTGAGGATGT
CATGTCTACCGGAACGGATATCATAATTGTCGGCCGAGGTCTGTACGGCCAGAACCG
AGATCCTATTGAGGAGGCCAAGCGATACCAGAAGGCTGGCTGGGAGGCTTACCAGA
AGATTAACTGTTAGAGGTTAGACTATGGATATGTCATTTAACTGTGTATATAGAGAG
CGTGCAAGTATGGAGCGCTTGTTCAGCTTGTATGATGGTCAGACGACCTGTCTGATC
GAGTATGTATGATACTGCACAACCTGCGCTTGAGACCATCCGGTCTCTGATCTTAAT
TAA
pLD139
(SEQ ID NO: 179)
TTAATTAATTCCGCTTAATGGAGTCCAAAAAGACCAACCTCTGCGCCTCGATCGACG
TGACCACAACCGCCGAGTTCCTTTCGCTCATCGACAAGCTCGGTCCCCACATCTGTC
TCGTGAAGACGCACATCGATATCATCTCAGACTTCAGCTACGAGGGCACGATTGAGC
CGTTGCTTGTGCTTGCAGAGCGCCACGGGTTCTTGATATTCGAGGACAGGAAGTTTG
CTGATATCGGAAACACCGTGATGTTGCAGTACACCTCGGGGGTATACCGGATCGCG
GCGTGGAGTGACATCACGAACGCGCACGGAGTGACTGGGAAGGGCGTCGTTGAAGG
GTTGAAACGCGGTGCGGAGGGGGTAGAAAAGGAAAGGGGCGTGTTGATGTTGGCG
GAGTTGTCGAGTAAAGGCTCGTTGGCGCATGGTGAATATACCCGTGAGACGATCGA
GATTGCGAAGAGTGATCGGGAGTTCGTGATTGGGTTCATCGCGCAGCGGGACATGG
GGGGTAGAGAAGAAGGGTTTGATTGGATCATCATGACGCCTGGTGTGGGGTTGGAT
GATAAAGGCGATGCGTTGGGCCAGCAGTATAGGACTGTTGATGAGGTGGTTCTGAC
TGGTACCGATGTGATTATTGTCGGGAGAGGGTTGTTTGGAAAAGGAAGAGACCCTG
AGGTGGAGGGAAAGAGATACAGGGATGCTGGATGGAAGGCATACTTGAAGAGAAC
TGGTCAGTTAGAATAAATATTGTAATAAATAGGTCTATATACATACACTAAGCTTCT
AGGACGTCATTGTAGTCTTCGAAGTTGTCTGCTAGTTTAGTTCTCATGATTTCGAAAA
CCAATAACGCAATGGATGTAGCAGGGATGGTGGTTAGTGCGTTCCTGACAAACCCA
GAGTACGCCGCCTCAAACCACGTCACATTCGCCCTTTGCTTCATCCGCATCACTTGCT
TGAAGGTATCCACGTACGAGTTGTAATACACCTTGAAGAACGGCTTCGTCTAGTTCG
GCATGGCAGATCATCATGCCTGCAGGAGCTCCAATTGTAATATTTCGGGAGAAATAT
CGTTGGGGTAAAACAACAGAGAGAGAGAGGGAGAGATGGTTCTGGTAGAATTATAA
TCTGGTTGTTGCAAATGCTACTGATCGACTCTGGCAATGTCTGTAGCTCGCTAGTTGT
ATGCAACTTAGGTGTTATGCATACACACGGTTATTCGGTTGAATTGTGGAGTAAAAA
TTGTCTGAGTTGTGTCTTAGCTACTGGCTGGCCCCCCGCGAAAGATAATCAAAATTA
CACTTGTGAATTTTTGCACACACACCGATTAACATTTCCCTTTTTTGTCCACCGATAC
ACGCTTGCCTCTTCTTATTTTCTCTGTGCTTCCCCCTCCTGTGACTTTTTCCACCATTG
ATATAAAATCAACTCCATTTCCCTAAAATCTCCCCAGATTCTAAAAACAACTTCTTCT
CTTCTGCTTTTCCTTATTTTTGTTATATTTATTTACCATCCCTTATTTTGAATAGTTATT
CCCCACTAACATTGTTCAAATCTTCACGACATAATGAACCCAAGGGAAAACTTCCTT
AAGTGTTTTCTGCAGTACATCCCTAACAATGCAACAAACCTCAAGTTGGTGTACACT
CAAAACAATCCACTCTATATGAGCGTGCTTAATAGCACAATCCACAACTTGCGCTTC
ACGTCAGATACTACGCCTAAGCCACTAGTGATCGTTACACCATCACACGTCAGCCAT
ATTCAAGGAACGATCCTATGTCTGAAAAAGGTCGGGTTGCAAATCAGGACTCGATC
AGGAGGGCACGATAGTGAGGGAATGAGTTACATCTCGCAAGTACCCTTCGTGATAG
TTGACTTGCGAAATATGCGGTCTATTAAAATTGACGTACATAGCCAGACCGCCTGGG
TTGAAGCAGGGGCAACCTTGGGTGAAGTTTATTACTGGGTCAATGAAAAAAACGAA
AACCTAAGTCTTGCTGCTGGATATTGCCCCACCGTTTGCGCGGGTGGTCATTTTGGA
GGCGGCGGATATGGTCCGTTGATGAGAAATTATGGACTTGCAGCAGACAATATTAT
AGATGCCCACTTGGTGAACGTTCATGGAAAGGTCTTGGACCGTAAGTCCATGGGTGA
AGATCTTTTCTGGGCCTTGAGAGGTGGTGGAGCGGAATCGTTTGGCATCATCGTTGC
CTGGAAAATTAGGTTGGTTGCGGTCCCGAAGAGTACAATGTTCTCCGTGAAGAAGAT
TATGGAAATACATGAGCTTGTCAAGTTAGTTAACAAGTGGCAAAATATCGCTTATAA
GTATGATAAAGACTTGCTTTTGATGACTCATTTTATTACGCGAAACATAACCGATAA
CCAGGGCAAGAACAAGACTGCTATTCACACGTACTTCTCCTCTGTATTTCTTGGAGG
AGTAGACTCCTTAGTTGACTTGATGAACAAGAGTTTCCCAGAATTGGGGATTAAGAA
GACAGATTGCAGACAATTATCGTGGATAGATACAATCATATTCTATAGCGGTGTCGT
CAATTACGATACTGATAATTTTAATAAAGAAATCCTCCTAGATCGTTCAGCTGGGCA
AAACGGGGCATTCAAAATTAAATTGGATTATGTGAAGAAACCAATTCCAGAGCTGG
TGTTTGTTCAGATATTGGAAAAACTTTACGAAGAAGACATTGGCGCAGGTATGTACG
CTTTGTATCCATATGGAGGCATTATGGACGAGATCTCAGAGCTGGCGATCCCCTTCC
CGCACAGAGCTGGGATACTCTACGAGCTATGGTACATCTGCTCTTGGGAGAAACAA
GAAGACAACGAGAAACATCTCAATTGGATTCGGAACATATACAACTTTATGACCCC
ATACGTATCAAAAAACCCGCGCTTAGCATACTTGAATTACAGAGACTTAGATATCGG
TATCAATGATCCTAAGAATCCTAACAATTACACCCAAGCCCGTATTTGGGGTGAGAA
ATATTTCGGCAAGAATTTTGACAGATTAGTTAAGGTCAAAACACTCGTGGACCCCAA
CAACTTTTTCCGAAACGAGCAGTCGATTCCACCACTACCCAGGCATAGACACGGAA
GAAGGGCAAAGTTGTAAGAGTGACTCTTTTGATAAGAGTCGCAAATTTGATTTCATA
AGTATATATTCATTATGTAAAGTAGTAAATGGAAAATTCATTAAAAAAAAAGCAAA
TTTCCGTTGTATGCATACTCCGAACACAAAACTAGCCCCGGAAAAACCCTTAGTTGA
TAGTTGCGAATTTAGGTCGACCATATGCGACGGGTACAACGAGAATTGTATTGAATT
GATCAAGAACATGATCTTGGTGTTACAGAACATCAAGTTCTTGGACCAGACTGAGA
ATGCACAGATATACAAGGCGTCATGTGATAAAATGGATGAGATTTATCCACAATTG
AAGAAAGAGTTTATGGAAAGTGGTCAACCAGAAGCTAAACAGGAAGAAGCAAACG
AAGAGGTGAAACAAGAAGAAGAAGGTAAATAAGTATTTTGTATTATATAACAAACA
AAGTAAGGAATACAGATTTATACAATAAATTGCCATACTAGTCACGTGAGATATCTC
ATCCATTCCCCAACTCCCAAGAAAATAAAAAAGTGAAAAATAAAATCAAACCCAAA
GATCAACCTCCCCATCATCATCGTCATCAAACCCCCAGCTCAATTCGCAATGGTTAG
CACAAAAACATACACAGAAAGGGCATCAGCACACCCCTCCAAGGTTGCCCAACGTT
TATTAATTAAAGGCTAGGTGGAGGCTCAGTGATGATAAGTCTGCGATGGTGGATGC
ATGTGTCATGGTCATAGCTGTTTCCTGTGTGAAATTGTTATCCGCTCAGAGGGCACA
ATCCTATTCCGCGCTATCCGACAATCTCCAAGACATTAGGTGGAGTTCAGTTCGGCG
TATGGCATATGTCGCTGGAAAGAACATGTGAGCAAAAGGCCAGCAAAAGGCCAGGA
ACCGTAAAAAGGCCGCGTTGCTGGCGTTTTTCCATAGGCTCCGCCCCCCTGACGAGC
ATCACAAAAATCGACGCTCAAGTCAGAGGTGGCGAAACCCGACAGGACTATAAAGA
TACCAGGCGTTTCCCCCTGGAAGCTCCCTCGTGCGCTCTCCTGTTCCGACCCTGCCGC
TTACCGGATACCTGTCCGCCTTTCTCCCTTCGGGAAGCGTGGCGCTTTCTCATAGCTC
ACGCTGTAGGTATCTCAGTTCGGTGTAGGTCGTTCGCTCCAAGCTGGGCTGTGTGCA
CGAACCCCCCGTTCAGCCCGACCGCTGCGCCTTATCCGGTAACTATCGTCTTGAGTC
CAACCCGGTAAGACACGACTTATCGCCACTGGCAGCAGCCACTGGTAACAGGATTA
GCAGAGCGAGGTATGTAGGCGGTGCTACAGAGTTCTTGAAGTGGTGGCCTAACTAC
GGCTACACTAGAAGAACAGTATTTGGTATCTGCGCTCTGCTGAAGCCAGTTACCTTC
GGAAAAAGAGTTGGTAGCTCTTGATCCGGCAAACAAACCACCGCTGGTAGCGGTGG
TTTTTTTGTTTGCAAGCAGCAGATTACGCGCAGAAAAAAAGGATCTCAAGAAGATCC
TTTGATCTTTTCTACGGGGTCTGACGCTCTATTCAACAAAGCCGCCGTCCCGTCAAGT
CAGCGTAAATGGGTAGGGGGCTTCAAATCGTCCGCTCTGCCAGTGTTACAACCAATT
AACAAATTCTGATTAGAAAAACTCATCGAGCATCAAATGAAACTGCAATTTATTCAT
ATCAGGATTATCAATACCATATTTTTGAAAAAGCCGTTTCTGTAATGAAGGAGAAAA
CTCACCGAGGCAGTTCCATAGGATGGCAAGATCCTGGTATCGGTCTGCGATTCCGAC
TCGTCCAACATCAATACAACCTATTAATTTCCCCTCGTCAAAAATAAGGTTATCAAG
TGAGAAATCACCATGAGTGACGACTGAATCCGGTGAGAATGGCAAAAGCTTATGCA
TTTCTTTCCAGACTTGTTCAACAGGCCAGCCATTACGCTCGTCATCAAAATCACTCGC
ATCAACCAAACCGTTATTCATTCGTGATTGCGCCTGAGCGAGACGAAATACGCGATC
GCTGTTAAAAGGACAATTACAAACAGGAATCGAATGCAACCGGCGCAGGAACACTG
CCAGCGCATCAACAATATTTTCACCTGAATCAGGATATTCTTCTAATACCTGGAATG
CTGTTTTCCCGGGGATCGCAGTGGTGAGTAACCATGCATCATCAGGAGTACGGATAA
AATGCTTGATGGTCGGAAGAGGCATAAATTCCGTCAGCCAGTTTAGTCTGACCATCT
CATCTGTAACATCATTGGCAACGCTACCTTTGCCATGTTTCAGAAACAACTCTGGCG
CATCGGGCTTCCCATACAATCGATAGATTGTCGCACCTGATTGCCCGACATTATCGC
GAGCCCATTTATACCCATATAAATCAGCATCCATGTTGGAATTTAATCGCGGCCTCG
AGCAAGACGTTTCCCGTTGAATATGGCTCATAACACCCCTTGTATTACTGTTTATGTA
AGCAGACAGTTTTATTGTTCATGATGATATATTTTTATCTTGTGCAATGTAACATCAG
AGATTTTGAGACACAACGTGGCTTTCCCCCGCCGCTCTAGAACTAGTGGATCCAAAT
AAAACGAAAGGCTCAGTCGAAAGACTGGGCCTTTCGTTTTATCTGTTGTTTGTCGCA
TTATACGAGACGTCCAGGTTGGGATACCTGAAACAAAACCCATCGTACGGCCAAGG
AAGTCTCCAATAACTGTGATCCACCACAAGCGCCAGGGTTTTCCCAGTCACGACGTT
GTAAAACGACGGCCAGTCATGCATAATCCGCACGCATCTGGAATAAGGAAGTGCCA
TTCCGCCTGACCT
pLD19
(SEQ ID NO: 180)
TTAATTAATTCCGCTTAATGGAGTCCAAAAAGACCAACCTCTGCGCCTCGATCGACG
TGACCACAACCGCCGAGTTCCTTTCGCTCATCGACAAGCTCGGTCCCCACATCTGTC
TCGTGAAGACGCACATCGATATCATCTCAGACTTCAGCTACGAGGGCACGATTGAGC
CGTTGCTTGTGCTTGCAGAGCGCCACGGGTTCTTGATATTCGAGGACAGGAAGTTTG
CTGATATCGGAAACACCGTGATGTTGCAGTACACCTCGGGGGTATACCGGATCGCG
GCGTGGAGTGACATCACGAACGCGCACGGAGTGACTGGGAAGGGCGTCGTTGAAGG
GTTGAAACGCGGTGCGGAGGGGGTAGAAAAGGAAAGGGGCGTGTTGATGTTGGCG
GAGTTGTCGAGTAAAGGCTCGTTGGCGCATGGTGAATATACCCGTGAGACGATCGA
GATTGCGAAGAGTGATCGGGAGTTCGTGATTGGGTTCATCGCGCAGCGGGACATGG
GGGGTAGAGAAGAAGGGTTTGATTGGATCATCATGACGCCTGGTGTGGGGTTGGAT
GATAAAGGCGATGCGTTGGGCCAGCAGTATAGGACTGTTGATGAGGTGGTTCTGAC
TGGTACCGATGTGATTATTGTCGGGAGAGGGTTGTTTGGAAAAGGAAGAGACCCTG
AGGTGGAGGGAAAGAGATACAGGGATGCTGGATGGAAGGCATACTTGAAGAGAAC
TGGTCAGTTAGAATAAATATTGTAATAAATAGGTCTATATACATACACTAAGCTTCT
AGGACGTCATTGTAGTCTTCGAAGTTGTCTGCTAGTTTAGTTCTCATGATTTCGAAAA
CCAATAACGCAATGGATGTAGCAGGGATGGTGGTTAGTGCGTTCCTGACAAACCCA
GAGTACGCCGCCTCAAACCACGTCACATTCGCCCTTTGCTTCATCCGCATCACTTGCT
TGAAGGTATCCACGTACGAGTTGTAATACACCTTGAAGAACGGCTTCGTCTAGTTCG
GCATGGCAGATCATCATGCCTGCAGGAGCTCCAATTGTAATATTTCGGGAGAAATAT
CGTTGGGGTAAAACAACAGAGAGAGAGAGGGAGAGATGGTTCTGGTAGAATTATAA
TCTGGTTGTTGCAAATGCTACTGATCGACTCTGGCAATGTCTGTAGCTCGCTAGTTGT
ATGCAACTTAGGTGTTATGCATACACACGGTTATTCGGTTGAATTGTGGAGTAAAAA
TTGTCTGAGTTGTGTCTTAGCTACTGGCTGGCCCCCCGCGAAAGATAATCAAAATTA
CACTTGTGAATTTTTGCACACACACCGATTAACATTTCCCTTTTTTGTCCACCGATAC
ACGCTTGCCTCTTCTTATTTTCTCTGTGCTTCCCCCTCCTGTGACTTTTTCCACCATTG
ATATAAAATCAACTCCATTTCCCTAAAATCTCCCCAGATTCTAAAAACAACTTCTTCT
CTTCTGCTTTTCCTTATTTTTGTTATATTTATTTACCATCCCTTATTTTGAATAGTTATT
CCCCACTAACATTGTTCAAATCTTCACGACataATGGGTTTATCGTCAGTGTGCACTTT
TTCTTTTCAAACAAACTACCACACCCTCCTAAACCCTCACAATAATAACCCAAAAAC
CTCCTTGCTATGTTACAGACATCCAAAGACACCGATCAAGTATTCATACAACAATTT
TCCCAGTAAACATTGCTCAACGAAGTCCTTCCACTTGCAAAACAAATGCAGCGAATC
ATTGTCGATAGCTAAAAACTCGATACGTGCGGCAACCACTAACCAAACTGAGCCAC
CAGAGAGCGATAATCATTCAGTCGCCACCAAGATTTTGAACTTTGGAAAAGCCTGTT
GGAAACTTCAAAGGCCTTACACCATTATCGCATTTACCAGTTGCGCATGTGGTTTGT
TCGGGAAGGAATTATTACACAACACAAATTTGATCAGCTGGAGCCTAATGTTTAAGG
CATTTTTCTTCTTAGTTGCAATTTTGTGTATAGCTTCGTTTACAACGACCATTAATCA
GATTTACGACCTTCACATCGATCGGATCAATAAACCAGACTTGCCCCTTGCCTCTGG
GGAAATCTCTGTAAATACTGCATGGATCATGCTGATAATCGTGGCTTTGTTTGGATT
GATTATTACAATTAAGATGAAGGGGGGTCCATTATATATATTCGGGTACTGCTTCGG
CATTTTCGGTGGTATCGTTTACTCCGTCCCACCCTTTAGATGGAAACAGAACCCCAG
TACGGCCTTTCTACTCAATTTCTTGGCTCATATCATCACAAACTTCACATTCTATTAT
GCAAGCCGAGCGGCGCTTGGTTTGCCGTTCGAACTCAGACCGAGTTTTACATTTCTC
CTTGCCTTCATGAAACTGATGGGACTGGCCCTTGCATTGATCAAGGATGCGTCAGAT
GTCGAAGGCGACACTAAGTTCGGCATTCTGACGCTTGCTTCCAAGTATGGAAGTAGA
AATCTAACGCTTTTTTGTTCAGGAATAGTGCTACTTAGTTATGTTGCTGCTATACTCG
CTGGCATTATTTGGCCTCAGGCCTTCAACTCTAACGTAATGTTGTTATCCCATGCTAT
TTTGGCGTTCTGGTTGATCTTGCAAACGCGAGATTTTGCACTCACTAACTACGACCC
AGAGGCAGGAAGGCGCTTTTACGAGTTTATGTGGAAGTTGTATTATGCCGAATACTT
GGTTTATGTTTTCATTTGATAAgagTGACTCTTTTGATAAGAGTCGCAAATTTGATTTC
ATAAGTATATATTCATTATGTAAAGTAGTAAATGGAAAATTCATTAAAAAAAAAGC
AAATTTCCGTTGTATGCATACTCCGAACACAAAACTAGCCCCGGAAAAACCCTTAGT
TGATAGTTGCGAATTTAGGTCGACCATATGCGACGGGTACAACGAGAATTGTATTGA
ATTGATCAAGAACATGATCTTGGTGTTACAGAACATCAAGTTCTTGGACCAGACTGA
GAATGCACAGATATACAAGGCGTCATGTGATAAAATGGATGAGATTTATCCACAAT
TGAAGAAAGAGTTTATGGAAAGTGGTCAACCAGAAGCTAAACAGGAAGAAGCAAA
CGAAGAGGTGAAACAAGAAGAAGAAGGTAAATAAGTATTTTGTATTATATAACAAA
CAAAGTAAGGAATACAGATTTATACAATAAATTGCCATACTAGTCACGTGAGATATC
TCATCCATTCCCCAACTCCCAAGAAAATAAAAAAGTGAAAAATAAAATCAAACCCA
AAGATCAACCTCCCCATCATCATCGTCATCAAACCCCCAGCTCAATTCGCAATGGTT
AGCACAAAAACATACACAGAAAGGGCATCAGCACACCCCTCCAAGGTTGCCCAACG
TTTATTAATTAAAGGCTAGGTGGAGGCTCAGTGATGATAAGTCTGCGATGGTGGATG
CATGTGTCATGGTCATAGCTGTTTCCTGTGTGAAATTGTTATCCGCTCAGAGGGCAC
AATCCTATTCCGCGCTATCCGACAATCTCCAAGACATTAGGTGGAGTTCAGTTCGGC
GTATGGCATATGTCGCTGGAAAGAACATGTGAGCAAAAGGCCAGCAAAAGGCCAGG
AACCGTAAAAAGGCCGCGTTGCTGGCGTTTTTCCATAGGCTCCGCCCCCCTGACGAG
CATCACAAAAATCGACGCTCAAGTCAGAGGTGGCGAAACCCGACAGGACTATAAAG
ATACCAGGCGTTTCCCCCTGGAAGCTCCCTCGTGCGCTCTCCTGTTCCGACCCTGCCG
CTTACCGGATACCTGTCCGCCTTTCTCCCTTCGGGAAGCGTGGCGCTTTCTCATAGCT
CACGCTGTAGGTATCTCAGTTCGGTGTAGGTCGTTCGCTCCAAGCTGGGCTGTGTGC
ACGAACCCCCCGTTCAGCCCGACCGCTGCGCCTTATCCGGTAACTATCGTCTTGAGT
CCAACCCGGTAAGACACGACTTATCGCCACTGGCAGCAGCCACTGGTAACAGGATT
AGCAGAGCGAGGTATGTAGGCGGTGCTACAGAGTTCTTGAAGTGGTGGCCTAACTA
CGGCTACACTAGAAGAACAGTATTTGGTATCTGCGCTCTGCTGAAGCCAGTTACCTT
CGGAAAAAGAGTTGGTAGCTCTTGATCCGGCAAACAAACCACCGCTGGTAGCGGTG
GTTTTTTTGTTTGCAAGCAGCAGATTACGCGCAGAAAAAAAGGATCTCAAGAAGATC
CTTTGATCTTTTCTACGGGGTCTGACGCTCTATTCAACAAAGCCGCCGTCCCGTCAAG
TCAGCGTAAATGGGTAGGGGGCTTCAAATCGTCCGCTCTGCCAGTGTTACAACCAAT
TAACAAATTCTGATTAGAAAAACTCATCGAGCATCAAATGAAACTGCAATTTATTCA
TATCAGGATTATCAATACCATATTTTTGAAAAAGCCGTTTCTGTAATGAAGGAGAAA
ACTCACCGAGGCAGTTCCATAGGATGGCAAGATCCTGGTATCGGTCTGCGATTCCGA
CTCGTCCAACATCAATACAACCTATTAATTTCCCCTCGTCAAAAATAAGGTTATCAA
GTGAGAAATCACCATGAGTGACGACTGAATCCGGTGAGAATGGCAAAAGCTTATGC
ATTTCTTTCCAGACTTGTTCAACAGGCCAGCCATTACGCTCGTCATCAAAATCACTCG
CATCAACCAAACCGTTATTCATTCGTGATTGCGCCTGAGCGAGACGAAATACGCGAT
CGCTGTTAAAAGGACAATTACAAACAGGAATCGAATGCAACCGGCGCAGGAACACT
GCCAGCGCATCAACAATATTTTCACCTGAATCAGGATATTCTTCTAATACCTGGAAT
GCTGTTTTCCCGGGGATCGCAGTGGTGAGTAACCATGCATCATCAGGAGTACGGATA
AAATGCTTGATGGTCGGAAGAGGCATAAATTCCGTCAGCCAGTTTAGTCTGACCATC
TCATCTGTAACATCATTGGCAACGCTACCTTTGCCATGTTTCAGAAACAACTCTGGC
GCATCGGGCTTCCCATACAATCGATAGATTGTCGCACCTGATTGCCCGACATTATCG
CGAGCCCATTTATACCCATATAAATCAGCATCCATGTTGGAATTTAATCGCGGCCTC
GAGCAAGACGTTTCCCGTTGAATATGGCTCATAACACCCCTTGTATTACTGTTTATGT
AAGCAGACAGTTTTATTGTTCATGATGATATATTTTTATCTTGTGCAATGTAACATCA
GAGATTTTGAGACACAACGTGGCTTTCCCCCGCCGCTCTAGAACTAGTGGATCCAAA
TAAAACGAAAGGCTCAGTCGAAAGACTGGGCCTTTCGTTTTATCTGTTGTTTGTCGC
ATTATACGAGACGTCCAGGTTGGGATACCTGAAACAAAACCCATCGTACGGCCAAG
GAAGTCTCCAATAACTGTGATCCACCACAAGCGCCAGGGTTTTCCCAGTCACGACGT
TGTAAAACGACGGCCAGTCATGCATAATCCGCACGCATCTGGAATAAGGAAGTGCC
ATTCCGCCTGACCT
pLD26
(SEQ ID NO: 181)
TTAATTAATTCCGCTTAATGGAGTCCAAAAAGACCAACCTCTGCGCCTCGATCGACG
TGACCACAACCGCCGAGTTCCTTTCGCTCATCGACAAGCTCGGTCCCCACATCTGTC
TCGTGAAGACGCACATCGATATCATCTCAGACTTCAGCTACGAGGGCACGATTGAGC
CGTTGCTTGTGCTTGCAGAGCGCCACGGGTTCTTGATATTCGAGGACAGGAAGTTTG
CTGATATCGGAAACACCGTGATGTTGCAGTACACCTCGGGGGTATACCGGATCGCG
GCGTGGAGTGACATCACGAACGCGCACGGAGTGACTGGGAAGGGCGTCGTTGAAGG
GTTGAAACGCGGTGCGGAGGGGGTAGAAAAGGAAAGGGGCGTGTTGATGTTGGCG
GAGTTGTCGAGTAAAGGCTCGTTGGCGCATGGTGAATATACCCGTGAGACGATCGA
GATTGCGAAGAGTGATCGGGAGTTCGTGATTGGGTTCATCGCGCAGCGGGACATGG
GGGGTAGAGAAGAAGGGTTTGATTGGATCATCATGACGCCTGGTGTGGGGTTGGAT
GATAAAGGCGATGCGTTGGGCCAGCAGTATAGGACTGTTGATGAGGTGGTTCTGAC
TGGTACCGATGTGATTATTGTCGGGAGAGGGTTGTTTGGAAAAGGAAGAGACCCTG
AGGTGGAGGGAAAGAGATACAGGGATGCTGGATGGAAGGCATACTTGAAGAGAAC
TGGTCAGTTAGAATAAATATTGTAATAAATAGGTCTATATACATACACTAAGCTTCT
AGGACGTCATTGTAGTCTTCGAAGTTGTCTGCTAGTTTAGTTCTCATGATTTCGAAAA
CCAATAACGCAATGGATGTAGCAGGGATGGTGGTTAGTGCGTTCCTGACAAACCCA
GAGTACGCCGCCTCAAACCACGTCACATTCGCCCTTTGCTTCATCCGCATCACTTGCT
TGAAGGTATCCACGTACGAGTTGTAATACACCTTGAAGAACGGCTTCGTCTAGTTCG
GCATGGCAGATCATCATGCCTGCAGGAGCTCCAATTGTAATATTTCGGGAGAAATAT
CGTTGGGGTAAAACAACAGAGAGAGAGAGGGAGAGATGGTTCTGGTAGAATTATAA
TCTGGTTGTTGCAAATGCTACTGATCGACTCTGGCAATGTCTGTAGCTCGCTAGTTGT
ATGCAACTTAGGTGTTATGCATACACACGGTTATTCGGTTGAATTGTGGAGTAAAAA
TTGTCTGAGTTGTGTCTTAGCTACTGGCTGGCCCCCCGCGAAAGATAATCAAAATTA
CACTTGTGAATTTTTGCACACACACCGATTAACATTTCCCTTTTTTGTCCACCGATAC
ACGCTTGCCTCTTCTTATTTTCTCTGTGCTTCCCCCTCCTGTGACTTTTTCCACCATTG
ATATAAAATCAACTCCATTTCCCTAAAATCTCCCCAGATTCTAAAAACAACTTCTTCT
CTTCTGCTTTTCCTTATTTTTGTTATATTTATTTACCATCCCTTATTTTGAATAGTTATT
CCCCACTAACATTGTTCAAATCTTCACGACataATGGCGGCAACCACTAACCAAACTG
AGCCACCAGAGAGCGATAATCATTCAGTCGCCACCAAGATTTTGAACTTTGGAAAA
GCCTGTTGGAAACTTCAAAGGCCTTACACCATTATCGCATTTACCAGTTGCGCATGT
GGTTTGTTCGGGAAGGAATTATTACACAACACAAATTTGATCAGCTGGAGCCTAATG
TTTAAGGCATTTTTCTTCTTAGTTGCAATTTTGTGTATAGCTTCGTTTACAACGACCA
TTAATCAGATTTACGACCTTCACATCGATCGGATCAATAAACCAGACTTGCCCCTTG
CCTCTGGGGAAATCTCTGTAAATACTGCATGGATCATGCTGATAATCGTGGCTTTGT
TTGGATTGATTATTACAATTAAGATGAAGGGGGGTCCATTATATATATTCGGGTACT
GCTTCGGCATTTTCGGTGGTATCGTTTACTCCGTCCCACCCTTTAGATGGAAACAGA
ACCCCAGTACGGCCTTTCTACTCAATTTCTTGGCTCATATCATCACAAACTTCACATT
CTATTATGCAAGCCGAGCGGCGCTTGGTTTGCCGTTCGAACTCAGACCGAGTTTTAC
ATTTCTCCTTGCCTTCATGAAACTGATGGGACTGGCCCTTGCATTGATCAAGGATGC
GTCAGATGTCGAAGGCGACACTAAGTTCGGCATTCTGACGCTTGCTTCCAAGTATGG
AAGTAGAAATCTAACGCTTTTTTGTTCAGGAATAGTGCTACTTAGTTATGTTGCTGCT
ATACTCGCTGGCATTATTTGGCCTCAGGCCTTCAACTCTAACGTAATGTTGTTATCCC
ATGCTATTTTGGCGTTCTGGTTGATCTTGCAAACGCGAGATTTTGCACTCACTAACTA
CGACCCAGAGGCAGGAAGGCGCTTTTACGAGTTTATGTGGAAGTTGTATTATGCCGA
ATACTTGGTTTATGTTTTCATTTGATAAgagTGACTCTTTTGATAAGAGTCGCAAATTT
GATTTCATAAGTATATATTCATTATGTAAAGTAGTAAATGGAAAATTCATTAAAAAA
AAAGCAAATTTCCGTTGTATGCATACTCCGAACACAAAACTAGCCCCGGAAAAACC
CTTAGTTGATAGTTGCGAATTTAGGTCGACCATATGCGACGGGTACAACGAGAATTG
TATTGAATTGATCAAGAACATGATCTTGGTGTTACAGAACATCAAGTTCTTGGACCA
GACTGAGAATGCACAGATATACAAGGCGTCATGTGATAAAATGGATGAGATTTATC
CACAATTGAAGAAAGAGTTTATGGAAAGTGGTCAACCAGAAGCTAAACAGGAAGA
AGCAAACGAAGAGGTGAAACAAGAAGAAGAAGGTAAATAAGTATTTTGTATTATAT
AACAAACAAAGTAAGGAATACAGATTTATACAATAAATTGCCATACTAGTCACGTG
AGATATCTCATCCATTCCCCAACTCCCAAGAAAATAAAAAAGTGAAAAATAAAATC
AAACCCAAAGATCAACCTCCCCATCATCATCGTCATCAAACCCCCAGCTCAATTCGC
AATGGTTAGCACAAAAACATACACAGAAAGGGCATCAGCACACCCCTCCAAGGTTG
CCCAACGTTTATTAATTAAAGGCTAGGTGGAGGCTCAGTGATGATAAGTCTGCGATG
GTGGATGCATGTGTCATGGTCATAGCTGTTTCCTGTGTGAAATTGTTATCCGCTCAGA
GGGCACAATCCTATTCCGCGCTATCCGACAATCTCCAAGACATTAGGTGGAGTTCAG
TTCGGCGTATGGCATATGTCGCTGGAAAGAACATGTGAGCAAAAGGCCAGCAAAAG
GCCAGGAACCGTAAAAAGGCCGCGTTGCTGGCGTTTTTCCATAGGCTCCGCCCCCCT
GACGAGCATCACAAAAATCGACGCTCAAGTCAGAGGTGGCGAAACCCGACAGGACT
ATAAAGATACCAGGCGTTTCCCCCTGGAAGCTCCCTCGTGCGCTCTCCTGTTCCGAC
CCTGCCGCTTACCGGATACCTGTCCGCCTTTCTCCCTTCGGGAAGCGTGGCGCTTTCT
CATAGCTCACGCTGTAGGTATCTCAGTTCGGTGTAGGTCGTTCGCTCCAAGCTGGGC
TGTGTGCACGAACCCCCCGTTCAGCCCGACCGCTGCGCCTTATCCGGTAACTATCGT
CTTGAGTCCAACCCGGTAAGACACGACTTATCGCCACTGGCAGCAGCCACTGGTAA
CAGGATTAGCAGAGCGAGGTATGTAGGCGGTGCTACAGAGTTCTTGAAGTGGTGGC
CTAACTACGGCTACACTAGAAGAACAGTATTTGGTATCTGCGCTCTGCTGAAGCCAG
TTACCTTCGGAAAAAGAGTTGGTAGCTCTTGATCCGGCAAACAAACCACCGCTGGTA
GCGGTGGTTTTTTTGTTTGCAAGCAGCAGATTACGCGCAGAAAAAAAGGATCTCAAG
AAGATCCTTTGATCTTTTCTACGGGGTCTGACGCTCTATTCAACAAAGCCGCCGTCCC
GTCAAGTCAGCGTAAATGGGTAGGGGGCTTCAAATCGTCCGCTCTGCCAGTGTTACA
ACCAATTAACAAATTCTGATTAGAAAAACTCATCGAGCATCAAATGAAACTGCAATT
TATTCATATCAGGATTATCAATACCATATTTTTGAAAAAGCCGTTTCTGTAATGAAG
GAGAAAACTCACCGAGGCAGTTCCATAGGATGGCAAGATCCTGGTATCGGTCTGCG
ATTCCGACTCGTCCAACATCAATACAACCTATTAATTTCCCCTCGTCAAAAATAAGG
TTATCAAGTGAGAAATCACCATGAGTGACGACTGAATCCGGTGAGAATGGCAAAAG
CTTATGCATTTCTTTCCAGACTTGTTCAACAGGCCAGCCATTACGCTCGTCATCAAAA
TCACTCGCATCAACCAAACCGTTATTCATTCGTGATTGCGCCTGAGCGAGACGAAAT
ACGCGATCGCTGTTAAAAGGACAATTACAAACAGGAATCGAATGCAACCGGCGCAG
GAACACTGCCAGCGCATCAACAATATTTTCACCTGAATCAGGATATTCTTCTAATAC
CTGGAATGCTGTTTTCCCGGGGATCGCAGTGGTGAGTAACCATGCATCATCAGGAGT
ACGGATAAAATGCTTGATGGTCGGAAGAGGCATAAATTCCGTCAGCCAGTTTAGTCT
GACCATCTCATCTGTAACATCATTGGCAACGCTACCTTTGCCATGTTTCAGAAACAA
CTCTGGCGCATCGGGCTTCCCATACAATCGATAGATTGTCGCACCTGATTGCCCGAC
ATTATCGCGAGCCCATTTATACCCATATAAATCAGCATCCATGTTGGAATTTAATCG
CGGCCTCGAGCAAGACGTTTCCCGTTGAATATGGCTCATAACACCCCTTGTATTACT
GTTTATGTAAGCAGACAGTTTTATTGTTCATGATGATATATTTTTATCTTGTGCAATG
TAACATCAGAGATTTTGAGACACAACGTGGCTTTCCCCCGCCGCTCTAGAACTAGTG
GATCCAAATAAAACGAAAGGCTCAGTCGAAAGACTGGGCCTTTCGTTTTATCTGTTG
TTTGTCGCATTATACGAGACGTCCAGGTTGGGATACCTGAAACAAAACCCATCGTAC
GGCCAAGGAAGTCTCCAATAACTGTGATCCACCACAAGCGCCAGGGTTTTCCCAGTC
ACGACGTTGTAAAACGACGGCCAGTCATGCATAATCCGCACGCATCTGGAATAAGG
AAGTGCCATTCCGCCTGACCT

References including disclosure related to compartmentalization of an enzyme through signal sequences include the following, each of which is incorporated by reference herein in its entirety: Agarwal et al. 2001 “Gene isolation and characterization of two acyl CoA oxidases from soybean with broad substrate specificities and enhanced expression in the growing seedling axis.” Plant Mol Biol. 2001 November; 47(4):519-31; Alconado and Juarez 2006. “Acyl-CoA oxidase activity from Beauveria bassiana, an entomopathogenic fungus”. J Basic Microbiol. 2006; 46(6):435-43; Aizpurua-Olaizola et al. “Identification and quantification of cannabinoids in Cannabis sativa L. plants by high performance liquid chromatography-mass spectrometry.” Anal Bioanal Chem. 2014 November; 406(29):7549-60; Backer et al. “Innovative development and validation of an HPLC/DAD method for the qualitative and quantitative determination of major cannabinoids in cannabis plant material.” J Chromatogr B Analyt Technol Biomed Life Sci. 2009 Dec. 15; 877(32):4115-24; Bakke et al. “N-ethylmaleimide-resistant acyl-coenzyme A oxidase from Arthrobacter ureafaciens NBRC 12140: molecular cloning, gene expression and characterization of the recombinant enzyme.” Biochim Biophys Acta. 2007 January; 1774(1):65-71; Barth and Gaillardin. “Physiology and genetics of the dimorphic fungus Yarrowia lipolytica.” FEMS Microbiol Rev. 1997 April; 19(4):219-37; Beggah et al. “Intra- and intermolecular events direct the propeptide-mediated maturation of the Candida albicans secreted aspartic proteinase Sap1p.” Microbiology. 2000 November; 146 (Pt 11):2765-73; Brocard and Hartig. “Peroxisome targeting signal 1: is it really a simple tripeptide?” Biochim Biophys Acta. 2006 December; 1763(12):1565-73; Brown et al. “Aspergillus has distinct fatty acid synthases for primary and secondary metabolism.: Proc Natl Acad Sci USA. 1996 Dec. 10; 93(25):14873-7; Carbalho et al. “Designing microorganisms for heterologous biosynthesis of cannabinoids.” FEMS Yeast Res. 2017 Jun. 1; 17(4); Gagne et al. “Identification of olivetolic acid cyclase from Cannabis sativa reveals a unique catalytic route to plant polyketides.” Proc Natl Acad Sci USA. 2012 Jul. 31; 109(31):12811-6; Gajewski et al. “Engineering fungal de novo fatty acid synthesis for short chain fatty acid production.” Nat Commun. 2017 Mar. 10; 8:14650; Gao et al. “Iterative integration of multiple-copy pathway genes in Yarrowia lipolytica for heterologous 0-carotene production”. Metab Eng. 2017 May; 41:192-201; Gietz and Woods “Transformation of Yeast by Lithium Acetate/Single-Stranded Carrier DNA/Polyethylene Glycol Method”. Methods Enzymol. 2002; 350:87-96.; Hong et al. “Engineering Yarrowia lipolytica to express secretory invertase with strong FBA1IN promoter”. Yeast. 2012 February; 29(2):59-72; Hooks et al. “Long-chain acyl-CoA oxidases of Arabidopsis.” Plant J. 1999 October; 20(1):1-13; Hunkova and Fenci. “Toxic effects of fatty acids on yeast cells: dependence of inhibitory effects on fatty acid concentration.” Biotechnol Bioeng. 1977 November; 19(11):1623-41; Kistler and Boz “Cellular compartmentalization of secondary metabolism” Front. Microbiology February 2015; Klionsky et al. “Intracellular sorting and processing of a yeast vacuolar hydrolase: proteinase A propeptide contains vacuolar targeting information.” Mol Cell Biol. 1988 May; 8(5):2105-16; Krink-Koutsoubelis et al.” Engineered Production of Short-Chain Acyl-Coenzyme A Esters in Saccharomyces cerevisiae.” ACS Synth Biol. 2018 Apr. 20; 7(4):1105-1115; Lametschwandtner et al. “The difference in recognition of terminal tripeptides as peroxisomal targeting signal 1 between yeast and human is due to different affinities of their receptor Pex5p to the cognate signal and to residues adjacent to it.” J Biol Chem. 1998 Dec. 11; 273(50):33635-43; Ledesma-Amaro and Nicaud. “Yarrowia lipolytica as a biotechnological chassis to produce usual and unusual fatty acids.” Prog Lipid Res. 2016 January; 61:40-50; Liang et al. “Structure, mechanism and function of prenyltransferase.” Eur J Biochem. 2002 July; 269(14):3339-54; Lui et al. “Membrane stress caused by octanoic acid in Saccharomyces cerevisiae” Appl Microbiol Biotechnol. 2013 April; 97(7):3239-51; Luo et. al 2002 “The acyl-CoA oxidases from the yeast Yarrowia lipolytica: characterization of Aox2p.” Arch Biochem Biophys. November 1; 407(1):32-8; Luo et al 2019 “Complete biosynthesis of cannabinoids and their unnatural analogues in yeast.” Nature. 2019 March; 567(7746):123-126; Pamplaniyi “Identification, isolation, and functional characterization of prenyltransferases in Cannabis sativa “Dissertation Dortmund 2016; Reiser et. al 2009 “AoxA is a major peroxisomal long chain fattyacyl-CoA oxidase required for beta-oxidation in A. nidulans”. Curr Genet. 2010 April; 56(2):139-50; Setoyama et. al 1995 “Functional expression of two forms of rat acyl-CoA oxidase and their substrate specificities” December 14; 217(2):482-7; Shimiu et al. “Type III Polyketide Synthases: Functional Classification and Phylogenomics. Chembiochem. 2017 Jan. 3; 18(1):50-65; Stout et al. “The hexanoyl-CoA precursor for cannabinoid biosynthesis is formed by an acyl-activating enzyme in Cannabis sativa trichomes.” Plant J. 2012 August; 71(3):353-65; Tan et al. “Synthetic Pathway for the Production of Olivetolic Acid in Escherichia coli”. ACS Synth Biol. 2018 Aug. 17; 7(8):1886-1896; Taura et al. “Characterization of olivetol synthase, a polyketide synthase putatively involved in cannabinoid biosynthetic pathway.” FEBS Lett. 2009 Jun. 18; 583(12):2061-6; Zirpel et al. “Production of Δ9-tetrahydrocannabinolic acid from cannabigerolic acid by whole cells of Pichia (Komagataella) pastoris expressing Δ9-tetrahydrocannabinolic acid synthase from Cannabis sativa L.” Biotechnol Lett. 2015 September; 37(9):1869-75; Zirpel et al. “Optimization of Δ9-tetrahydrocannabinolic acid synthase production in Komagataella phaffii via post-translational bottleneck identification.” J Biotechnol. 2018 Apr. 20; 272-273:40-47; Zirpel “Recombinant Expression and Functional Characterization of Cannabinoid Producing Enzymes in Komagataella phaffii” Dissertation Dortmund 2018; Yang et al “Structural basis for olivetolic acid formation by a polyketide cyclase from Cannabis sativa.” FEBS J. 2016 March; 283(6):1088-106; U.S. Pat. Nos. 7,851,199; 8,884,100; 9,546,362; 9,611,460; 9,765,308; 9,822,384; 10,059,971; 10,287,557; U.S. Pat. Publ. No. 2014/0228586 A1; U.S. Pat. Publ. No. 2016/0010126 A1; U.S. Pat. Publ. No. 2016/0298151 A1; U.S. Pat. Publ. No. 2017/0211049 A1; U.S. Pat. Publ. No. 2018/0073043 A1; U.S. Pat. Publ. No. 2018/0155748 A1; U.S. Pat. Publ. No. 2018/0334692A1; PCT Publ. No. WO2017139496A1; PCT Publ. No. WO2018200888A1; PCT Publ. No. WO2018219995A1; PCT Publ. No. WO2018148849A1; PCT Publ. No. WO2018148848A1; and PCT Publ. No. WO2019071000A1.

### Exemplary Methods, Microorganisms, and Compositions (e.g., Polyketides)

Method 1: A method, comprising: providing a microorganism selected from the group consisting of a fungi and a yeast, wherein the microorganism has been modified to produce a polyketide in fermentation, wherein at least one of the enzymes that mediate the polyketide production have been targeted to at least one compartment within a secretory pathway resulting in an increase in secretion of the polyketide.

Method 2: Method 1, wherein the polyketide is a cannabinoid.

Method 3: Method 2, wherein the poleketide is a cannabinoid that is selected from the group consisting of cannabigerolic acid, Δ9-tetrahydrocannabinolic acid, cannabidiolic acid, cannabichromenic acid, cannabigerovarinic acid, tetrahydrocannabivarin acid, cannabidivarinic acid, and cannabichromevarinic acid.

Method 4: Method 1, wherein the polyketide is olivetolic acid.

Method 5: Method 1, wherein the microorganism is a yeast.

Method 6: Method 5, wherein the yeast is from a genus selected from the group consisting of Candida, Arxula, Pichia, Scheffersomyces, Kluyveromyces, Saccharomyces, Yarrowia, or Schizosaccharomyces.

Method 7: Method 5, wherein the yeast is a Candida viswanathii.

Method 8: Method 5, wherein the yeast is Arxula adeninivorans.

Method 9: Method 5, wherein the yeast is Yarrowia lipolytica.

Method 10: Method 1, wherein the microorganism is a fungi.

Method 11: Method 10, wherein the fungi is from a genus selected from the group consisting of Aspergillus, Trichoderma or Myceliophthora.

Method 12: Method 10, wherein the fungi is Aspergillus niger.

Method 13: Method 10, wherein the fungi is Aspergillus terreus.

Method 14: Method 10, wherein the fungi is Trichoderma harzianum.

Method 15: Method 10, wherein the fungi is Myceliophthora thermophila.

Method 16: Method 1, wherein the compartment within the secretory pathway is an endoplasmic reticulum.

Method 17: Method 1, wherein the compartment within the secretory pathway is a Golgi apparatus.

Method 18: Method 1, wherein the compartment within the secretory pathway is a vacuole.

Method 19: Method 1, wherein the compartment within the secretory pathway is an endosome.

Method 20: Method 1, wherein the enzyme is removed of its endogenous amino-terminal localization sequence and/or carboxyl-terminal localization sequence.

Method 21: Method 16, whereby the enzyme has been modified with the N-terminal addition of the N-terminal 24 amino acids (MMWKFLIAIGHLIFSYCCNAQLLDS) from OST1 and a C-terminal addition of the amino acids HDEL to localize the enzyme to the endoplasmic reticulum.

Method 22: Method 18, whereby the enzyme has been modified by an N-terminal addition of N-terminal 73 amino acids (MGITNETQALLGGDSLSCLNKKKSNTKRNLSYLLNIITVSfIAYLCFFATHNHHNDSGIPK VDPHKKKNIIMM) of PHO8 to localize the enzyme to the vacuole.

Method 23: Method 18, whereby the enzyme has been modified with the N-terminal fusion with Sna4 to localize the enzyme to the vacuole.

Method 24: Method 18, whereby the enzyme has been modified with the N-terminal addition of N-terminal 92 amino acids (MQLSLSVLSTVATALLSLTTAVDAKSHNIKLSKLSNEETLDASTFQEYTSSLANKYMNL FNAAHGNPTSFGLQHVLSNQEAEVPFVTPQKGG) from vacuolar aspartic protease to localize the enzyme to the vacuole.

Method 25: Method 19, whereby the enzyme has been modified with the N-terminal addition of N-terminal 305 amino acids from DOA4 (MTLLLKPTSELDAGSRKIIERIQSNSPTFQHLFDLLLNLLPFFDKTVSLLSIGYCDYEVA YVTYQTCIQVVGLMKPKTNSLNQDIFKGVQLQTRKRASTFKAILSYFAEPETQEEDPLLN RFKSLSGGGSKTKSSQDEVFHEWITSSELQRLSSKKVLLIDFRPRKDYLNNHIKYKDLV HIEPTQLETLLDSASDQDLETLVKKSAPYDQYHIFLERHKYDLIVVYNYNYGSESTDRLL GIIDVVSKPNPFTKLITILMNNKYISSRLKVKPLFLSGGVLNWYKTFGIEYLERTLVQNGV AHT) to localize the enzyme to an endosomal compartment.

Method 26: Method 17, whereby the enzyme has been modified with the N-terminal addition of N-terminal 81 amino acids of beta-galactosyltransferase (MRLREPIISGSAAMPGASLQRACRIIVAVCALIILGVTLVYYLAGRDLSRLPQLVGVS TPLQGGSNSAAAIGQSSGELRTGG) to localize the enzyme to a Golgi compartment.

Method 27: Method 17, whereby the enzyme has been modified with N-terminal 34 amino acids of rat liver alpha-2,6-sialyltransferase (MIHTNLKKKFSLFILVFLLFAVICVWKKGSDYEA) to localize the enzyme to a Golgi compartment.

Method 28: Method 16 whereby the enzyme has been modified the N-terminal addition of the N-terminal 31 amino acids (MKFGVLFSVFAAIVSALPLQEGPLNKRAYPS) from glucoamylase and a C-terminal addition of the amino acids HDEL to localize the enzyme to the endoplasmic reticulum.

Method 29: Method 1, wherein the fermentation uses a fatty acid and/or sugar as a carbon source.

Method 30: Method 1, wherein the fermentation uses dextrose or sucrose as a carbon source.

Method 31: Method 1, wherein the enzyme is prenyltransferase.

Method 32: Method 1, wherein the enzyme is cannabidiolic acid synthase.

Method 33: Method 32, wherein the microorganism has been modified to express the protein sequence SEQ. ID *53, 54, 55 or 56.

Method 34: Method 1, wherein the enzyme is tetrahydrocannabidiolic acid synthase.

Method 35: Method 34, wherein the microorganism has been modified to express the protein sequence SEQ. ID *58, 59, 60 or 61.

Method 36: Method 1, wherein the enzyme is cannabichromenic acid synthase.

Microorganism 37: A microorganism selected from the group consisting of a fungi and a yeast, wherein the microorganism has been modified to produce a polyketide in fermentation, wherein at least one of the enzymes that mediate the polyketide production have been targeted to at least one compartment within a secretory pathway resulting in an increase in secretion of the one polyketide.

Microorganism 38: Microorganism 37, wherein the microorganism is a yeast.

Microorganism 39: Microorganism 38, wherein the yeast is from a genus selected from the group consisting of Candida, Arxula, Pichia, Scheffersomyces, Kluyveromyces, Saccharomyces, Yarrowia, or Schizosaccharomyces.

Microorganism 40: Microorganism 38, wherein the yeast is a Candida viswanathii.

Microorganism 41: Microorganism 38, wherein the yeast is Arxula adeninivorans.

Microorganism 42: Microorganism 38, wherein the yeast is Yarrowia lipolytica.

Microorganism 43: Microorganism 37, wherein the microorganism is a fungi.

Microorganism 44: Microorganism 43, wherein the fungi is from a genus selected from the group consisting of Aspergillus, Trichoderma or Myceliophthora.

Microorganism 45: Microorganism 43, wherein the fungi is Aspergillus niger.

Microorganism 46: Microorganism 43, wherein the fungi is Aspergillus terreus.

Microorganism 47: Microorganism 43, wherein the fungi is Trichoderma harzianum.

Microorganism 48: Microorganism 43, wherein the fungi is Myceliophthora thermophila.

Microorganism 49: Microorganism 37, wherein the polyketide is a cannabinoid.

Microorganism 50: Microorganism 37, wherein the polyketide is selected from the group consisting of cannabigerolic acid, Δ9-tetrahydrocannabinolic acid, cannabidiolic acid, cannabichromenic acid, cannabigerovarinic acid, tetrahydrocannabivarin acid, cannabidivarinic acid, and cannabichromevarinic acid.

Microorganism 51: Microorganism 37, wherein the polyketide is olivetolic acid.

Microorganism 52: Microorganism 37, wherein the compartment within the secretory pathway is an endoplasmic reticulum.

Microorganism 53: Microorganism 37, wherein the compartment within the secretory pathway is a Golgi apparatus.

Microorganism 54: Microorganism 37, wherein the compartment within the secretory pathway is a vacuole.

Microorganism 55: Microorganism 37, wherein the compartment within the secretory pathway is an endosome.

Microorganism 56: Microorganism 37, wherein the enzyme is removed of its endogenous amino-terminal localization sequence and/or carboxyl-terminal localization sequence.

Microorganism 57: Microorganism 52, whereby the enzyme has been modified with the N-terminal addition of the N-terminal 24 amino acids (MMWKFLIAIGLIFSYCCNAQLLDS) from OST1 and a C-terminal addition of the amino acids HDEL to localize the enzyme to the endoplasmic reticulum.

Microorganism 58: Microorganism 54, whereby the enzyme has been modified by an N-terminal addition of N-terminal 73 amino acids (MGITNETQALLGGDSLSCLNKKKSNTKRNLSYLLNIITVSIIAYLCFFATHNHHNDSGIPK VDPHKKKNIMM) of PHO8 to localize the enzyme to the vacuole.

Microorganism 59: Microorganism 54, whereby the enzyme has been modified with the N-terminal fusion with Sna4 to localize the enzyme to the vacuole.

Microorganism 60: Microorganism 54, whereby the enzyme has been modified with the N-terminal addition of N-terminal 92 amino acids (MQLSLSVLSTVATALLSLTTAVDAKSHNIKLSKLSNEETLDASTFQEYTSSLANKYMNL FNAAHGNPTSFGLQHVLSNQEAEVPFVTPQKGG) from vacuolar aspartic protease to localize the enzyme to the vacuole.

Microorganism 61: Microorganism 55, whereby the enzyme has been modified with the N-terminal addition of N-terminal 305 amino acids from DOA4 (MTLLLIKPTSELDATSRKlIERIQSNSPTFQI-ILFDLLLNLLPFFDKTVSLLGSIGYCDYEVA YVTYQTCIQVVGLMKPKTNSLNQDIFKGVQLQTRKRASTFKAILSYFAEPETQEEDPLLN RFKSLSGGGSKTKSSQDEVFHEWITSSELQRELSSKKVIILDFRPRKDYLNNHIKYKDLV HIEPTQLETLLDSASDQDLETLVKKSAPYDQYHIFLERHKYDLIVVYNYNYGSESTDRLL GIIDVVSKPNPFTKLITILMNNKYISSRLKVKPLFLSGGVLNWYKTFGIEYLERTLVQNGV AHT) to localize the enzyme to an endosomal compartment.

Microorganism 62: Microorganism 53, whereby the enzyme has been modified with the N-terminal addition of N-terminal 81 amino acids of beta-galactosyltransferase (MRLREPLLSGSAAMPGASLQRACRLLVAVCALHLGVTLVYYLAGRDLSRLPQLVGVS TPLQGGSNSAAAIGQSSGELRTGG) to localize the enzyme to a Golgi compartment.

Microorganism 63: Microorganism 53, whereby the enzyme has been modified with N-terminal 34 amino acids of rat liver alpha-2,6-sialyltransferase (MIHTNLKKKFSLFILVFLLFAVICVWKKGSDYEA) to localize the enzyme to a Golgi compartment.

Microorganism 64: Microorganism 52, whereby the enzyme has been modified the N-terminal addition of the N-terminal 31 amino acids (MKFGVLFSVFAAIVSALPLQEGPLNKRAYPS) from glucoamylase and a C-terminal addition of the amino acids HDEL to localize the enzyme to the endoplasmic reticulum.

Microorganism 65: Microorganism 37, wherein the fermentation uses a fatty acid and/or sugar as a carbon source.

Microorganism 66: Microorganism 37, wherein the fermentation uses dextrose or sucrose as a carbon source.

Microorganism 67: Microorganism 37, wherein the enzyme is prenyltransferase.

Microorganism 68: Microorganism 37, wherein the enzyme is cannabidiolic acid synthase.

Microorganism 69: Microorganism 68, wherein the microorganism has been modified to express the protein sequence SEQ. ID *53, 54, 55 or 56.

Microorganism 70: Microorganism 37, wherein the enzyme is tetrahydrocannabidiolic acid synthase.

Microorganism 71: Microorganism 70, wherein the microorganism has been modified to express the protein sequence SEQ. ID *58, 59, 60 or 61.

Microorganism 72: Microorganism 37, wherein the enzyme is cannabichromenic acid synthase.

Polyketide 73: A polyketide produced by any one of Methods 1-36.

Polyketide 74: A polyketide produced by any one of Microorganisms 37-72.

Method 75: A method, comprising: providing as a feedstock at least one of a fatty acid, vegetable oil, or an alkane to a microorganism which has a modified beta-oxidation pathway, wherein the beta-oxidation pathway has been modified by a modification to produce a fatty acid or a fatty acid-CoA that is a substrate for an acyl-CoA synthase or a polyketide synthase, respectively, wherein the microorganism produces a polyketide.

Method 76: Method 75, wherein at least one of an acyl-coA oxidase, enoyl CoA hydratase, 3-hydroxyacyl-CoA dehydrogenase, B-ketothiolase or thiolase has been modified and/or replaced in the microorganism.

Method 77: Method 75, wherein the modification is one or more of a deletion, a mutation, a replacement, or an expression of one of acyl-coA oxidase, enoyl CoA hydratase, 3-hydroxyacyl-CoA dehydrogenase, B-ketholiase, or thiolase.

Method 78: Method 75, wherein the modification is of acyl-coA oxidase.

Method 79: Method 75, wherein the microorganism has been modified to include wherein the microorganism has been modified to express the protein sequence SEQ. ID 10.

Method 80: Method 75, wherein the feedstock is a fatty acid or a vegetable oil.

Method 81: Method 80, wherein the fatty acid is selected from the group consisting of oleic acid, palmitic acid, stearic acid, linoleic acid, alpha-linolenic acid, palmitoleic acid, tridecanoic acid, pentadecanoic acid, and nonanoic acid.

Method 82: Method 80, wherein the vegetable oil is selected from the group consisting of palm oil, soybean oil, corn oil, canola oil, coconut oil, sunflower oil, olive oil, palm kernel oil, lard, castor oil, peanut oil, sesame oil, grapeseed, avocado oil, and flaxseed oil.

Method 83: Method 75, wherein the fatty acid is in a form of a methyl ester or ethyl ester.

Method 84: Method 75, wherein the alkane is selected from the group consisting of octadecane, hetadecane, hexadecane, pentadecane, tetradecane, tridecane, dodecane, and undecane.

Method 85: Method 75, wherein the polyketide synthase is a tetraketyde synthase.

Method 86: Method 75, wherein the polyketide synthase is TKS1 or TKS1p.

Method 87: Method 75, wherein the polyketide synthase is targeted to the peroxisome.

Method 88: Method 75, wherein an olivetolic acid synthase is expressed.

Method 89: Method 88, wherein the olivetolic acid synthase is CsOAC1.

Method 90: Method 88, where the olivetolic acid synthase is targeted to the peroxisome.

Method 91: Method 75, wherein an endogenous acyl-coA oxidase has been mutated.

Method 92: Method 75, wherein an endogenous acyl-coA oxidase has been replaced with a non-native acyl-coA oxidase.

Method 93: Method 92, wherein the non-native acyl-coA oxidase is selected from the group consisting of ACO1P, ACO2, ACO3, ACO4, ACO5, ACO6, ACO7, ACO8, ACO9 and ACO10.

Method 94: Method 75, wherein the polyketide is a cannabinoid.

Method 95: Method 75, wherein the polyketide is selected from the group consisting of cannabigerolic acid, Δ9-tetrahydrocannabinolic acid, cannabidiolic acid, cannabichromenic acid, cannabigerovarinic acid, tetrahydrocannabivarin acid, cannabidivarinic acid, and cannabichromevarinic acid.

Method 96: Method 75, wherein the polyketide is olivetolic acid.

Method 97: Method 75, wherein a polyketide synthase is expressed.

Method 98: Method 97, wherein the polyketide synthase is a tetraketyde synthase.

Method 99: Method 97, wherein the polyketide synthase is TKS1 or TKS1p

Method 100: Method 97, wherein the polyketide synthase is targeted to the peroxisome.

Method 101: Method 75, wherein an olivetolic acid synthase is expressed.

Method 102: Method 101, wherein the olivetolic acid synthase is CsOAC1.

Method 103: Method 101, where the olivetolic acid synthase is targeted to the peroxisome.

Method 104: Method 75, wherein beta-oxidation has been modified.

Method 105: Method 104, wherein an endogenous acyl-coA oxidase has been mutated.

Method 106: Method 104, wherein an endogenous acyl-coA oxidase has been replaced with a non-native acyl-coA oxidase.

Method 107: Method 106, wherein the non-native acyl-coA oxidase is selected from the group consisting of ACO1P, ACO2, ACO3, ACO4, ACO5, ACO6, ACO7, ACO8, ACO9, and ACO10.

Method 108: Method 75, wherein the microorganism is a yeast.

Method 109: Method 108, wherein the yeast is from a genus selected from the group consisting of Candida, Arxula, Pichia, Scheffersomyces, Kluyveromyces, Saccharomyces, Yarrowia, or Schizosaccharomyces.

Method 110: Method 108, wherein the yeast is a Candida viswanathii.

Method 111: Method 108, wherein the yeast is Arxula adeninivorans.

Method 112: Method 108, wherein the yeast is Yarrowia lipolytica.

Method 113: Method 75, wherein the microorganism is a fungi.

Method 114: Method 113, wherein the fungi is from a genus selected from the group consisting of Aspergillus, Trichoderma or Myceliophthora.

Method 115: Method 113, wherein the fungi is Aspergillus niger.

Method 116: Method 113, wherein the fungi is Aspergillus terreus.

Method 117: Method 113, wherein the fungi is Trichoderma harzianum.

Method 118: Method 113, wherein the fungi is Myceliophthora thermophila.

Microorganism 119: A microorganism, wherein the microorganism is a yeast or a fungi having a modified beta-oxidation pathway, wherein the beta-oxidation pathway has been modified by a modification to produce a fatty acid or a fatty acid-CoA that is a substrate for an acyl-CoA synthase or a polyketide synthase, respectively, wherein the microorganism produces a polyketide.

Microorganism 120: Microorganism 119, wherein the microorganism is a yeast.

Microorganism 121: Microorganism 120, wherein the yeast is from a genus selected from the group consisting of Candida, Arxula, Pichia, Scheffersomyces, Kluyveromyces, Saccharomyces, Yarrowia, or Schizosaccharomyces.

Microorganism 122: Microorganism 120, wherein the yeast is a Candida viswanathii.

Microorganism 123: Microorganism 120, wherein the yeast is Arxula adeninivorans.

Microorganism 124: Microorganism 120, wherein the yeast is Yarrowia lipolytica.

Microorganism 125: Microorganism 119, wherein the microorganism is a fungi.

Microorganism 126: Microorganism 125, wherein the fungi is from a genus selected from the group consisting of Aspergillus, Trichoderma or Myceliophthora.

Microorganism 127: Microorganism 125, wherein the fungi is Aspergillus niger.

Microorganism 128: Microorganism 125, wherein the fungi is Aspergillus terreus.

Microorganism 129: Microorganism 125, wherein the fungi is Trichoderma harzianum.

Microorganism 130: Microorganism 125, wherein the fungi is Myceliophthora thermophila.

Microorganism 131: Microorganism 119, wherein at least one of an acyl-coA oxidase, enoyl CoA hydratase, 3-hydroxyacyl-CoA dehydrogenase, B-ketholiase or thiolase has been modified and/or replaced in the microorganism.

Microorganism 132: Microorganism 119, wherein the modification is one or more of a deletion, a mutation, a replacement, or an expression of one of acyl-coA oxidase, enoyl CoA hydratase, 3-hydroxyacyl-CoA dehydrogenase, B-ketholiase, or thiolase.

Microorganism 133: Microorganism 119, wherein the modification is of acyl-coA oxidase.

Microorganism 134: Microorganism 119, wherein the microorganism has been modified to include SEQ. ID 10.

Microorganism 135: Microorganism 119, wherein the polyketide synthase is a tetraketyde synthase.

Microorganism 136: Microorganism 119, wherein the polyketide synthase is TKS1 or TKS1p.

Microorganism 137: Microorganism 119, wherein the polyketide synthase is targeted to the peroxisome.

Microorganism 138: Microorganism 119, wherein an olivetolic acid synthase is expressed.

Microorganism 139: Microorganism 138, wherein the olivetolic acid synthase is CsOAC1.

Microorganism 140: Microorganism 119, where the olivetolic acid synthase is targeted to the peroxisome.

Microorganism 141: Microorganism 119, wherein an endogenous acyl-coA oxidase has been mutated.

Microorganism 142: Microorganism 119, wherein an endogenous acyl-coA oxidase has been replaced with a non-native acyl-coA oxidase.

Microorganism 143: Microorganism 142, wherein the non-native acyl-coA oxidase is selected from the group consisting of ACO1P, ACO2, ACO3, ACO4, ACO5, ACO6, ACO7, ACO8, ACO9, and ACO10.

Microorganism 144: Microorganism 119, wherein the polyketide is a cannabinoid.

Microorganism 145: Microorganism 119, wherein the polyketide is selected from the group consisting of cannabigerolic acid, Δ9-tetrahydrocannabinolic acid, cannabidiolic acid, cannabichromenic acid, cannabigerovarinic acid, tetrahydrocannabivarin acid, cannabidivarinic acid, and cannabichromevarinic acid.

Microorganism 146: Microorganism 119, wherein the polyketide is olivetolic acid.

Polyketide 147: A polyketide produced by any one of Methods 75-118.

Polyketide 148: A polyketide produced by any one of Microorganisms 119-146.

Method 149: A method, comprising: providing a microorganism that has been modified to produce a polyketide; and providing the microorganism with a C5-11 fatty acid ester in fermentation to yield a corresponding C5-9 fatty acid, wherein the C5-9 fatty acid is activated by an Acyl-CoA synthase and is used as a substrate for a polyketide synthase, from which the polyketide is obtained.

Method 150: Method 149, wherein the microorganism has been genetically modified.

Method 151: Method 149, wherein the polyketide is a cannabinoid.

Method 152: Method 151, wherein the cannabinoid is selected from the group consisting of cannabigerolic acid, Δ9-tetrahydrocannabinolic acid, cannabidiolic acid, cannabichromenic acid, cannabigerovarinic acid, tetrahydrocannabivarin acid, cannabidivarinic acid, and cannabichromevarinic acid.

Method 153: Method 149, wherein the polyketide is olivetolic acid.

Method 154: Method 149, wherein the microorganism is a yeast.

Method 155: Method 154, wherein the yeast is from a genus selected from the group consisting of Candida, Arxula, Pichia, Scheffersomyces, Kluyveromyces, Saccharomyces, Yarrowia, or Schizosaccharomyces.

Method 156: Method 154, wherein the yeast is a Candida viswanathii.

Method 157: Method 154, wherein the yeast is Arxula adeninivorans.

Method 158: Method 154, wherein the yeast is Yarrowia lipolytica.

Method 159: Method 149, wherein the microorganism is a fungi.

Method 160: Method 159, wherein the fungi is from a genus selected from the group consisting of Aspergillus, Trichoderma or Myceliophthora.

Method 162: Method 159, wherein the fungi is Aspergillus niger.

Method 163: Method 159, wherein the fungi is Aspergillus terreus.

Method 164: Method 159, wherein the fungi is Trichoderma harzianum.

Method 164: Method 159, wherein the fungi is Myceliophthora thermophila.

Method 165: Method 159, wherein the C₅₋₁₁ fatty acid ester is a C₆ fatty acid ester.

Method 166: Method 159, wherein the C₅₋₁₁ fatty acid ester is an ester of an acid selected from the group consisting of pentanoic acid, hexanoic acid, heptanoic acid, octanoic acid, nonanoic acid, decanoic acid, and undecanoic acid.

Method 167: Method 159, wherein the C₅₋₁₁ fatty acid ester is an ester selected from the group consisting of methyl ester, ethyl ester, and genaryl ester.

Polyketide 168: A polyketide produced by any one of Methods 149-167.

The above description presents the best mode contemplated for carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and “one or more’ to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

1. (canceled)

2. A method of producing a compound in a peroxisome, the method comprising:
providing a fatty acid to a genetically modified microorganism, wherein the genetically modified organism is a yeast or a fungus, wherein the microorganism comprises a biosynthetic enzyme targeted to the peroxisome, wherein the biosynthetic enzyme is capable of catabolizing the fatty acid into the compound;
culturing the microorganism under conditions sufficient to produce the compound in the peroxisome; and
isolating the compound.

3. The method of claim 2, wherein the compound is a fatty acyl-CoA.

4. The method of claim 3, wherein the fatty acyl-CoA is acetyl-CoA, malonyl-CoA, or hexanoyl-CoA.

5. The method of claim 2, wherein the yeast is from a genus selected from the group consisting of Candida, Arxula, Pichia, Scheffersomyces, Kluyveromyces, Saccharomyces, Yarrowia, or Schizosaccharomyces.

6. The method of claim 2, wherein the fungus is Aspergillus parasiticus, Aspergillus nidulans, Thraustochytrium, Schizochytrium, Rhizopus arrhizus, Rhizopus oryzae, or Rhizopus nigricans.

7. The method of claim 2, wherein the biosynthetic enzyme is a fatty acyl-CoA synthetase, fatty acyl activating enzyme, acyl-CoA oxidase, acyl-CoA thioesterase, hexanoyl-CoA synthetase, acetyl-CoA carboxylase, enoyl-CoA hydratase, 3-hydroxyacyl-CoA dehydrogenase, beta-ketothiolase, thiolase, acyl-CoA synthase, cannabidiolic acid synthase, tetrahydrocannabidiolic acid, olivetolic acid synthase, or polyketide synthase.

8. The method of claim 7, wherein the polyketide synthase is a Type III polyketide synthase.

9. The method of claim 8, wherein the Type III polyketide synthase is tetraketide synthase, TKS1, or TKS1p.

10. The method of claim 2, wherein the biosynthetic enzyme comprises a peroxisomal targeting sequence.

11. The method of claim 10, wherein the peroxisomal targeting sequence has a consensus sequence of [S/A/H/C/E/P/Q/V]-[K/R/H/Q]-[L/F] as set forth in SEQ ID NO: 7, or GRRAKL as set forth in SEQ ID NO: 6.

12. The method of claim 2, wherein the enzyme is removed of its endogenous amino-terminal localization sequence and/or carboxyl-terminal localization sequence.

13. A genetically modified microorganism, comprising a biosynthetic enzyme targeted to a peroxisome in the microorganism, wherein the genetically modified microorganism is a yeast or a fungus, wherein the biosynthetic enzyme is capable of catabolizing a fatty acid into a compound of interest.

14. The genetically modified microorganism of claim 13, wherein the yeast is from a genus selected from the group consisting of Candida, Arxula, Pichia, Scheffersomyces, Kluyveromyces, Saccharomyces, Yarrowia, or Schizosaccharomyces.

15. The genetically modified microorganism of claim 13, wherein the fungus is Aspergillus parasiticus, Aspergillus nidulans, Thraustochytrium, Schizochytrium, Rhizopus arrhizus, Rhizopus oryzae, or Rhizopus nigricans.

16. The genetically modified microorganism of claim 13, wherein the biosynthetic enzyme is a fatty acyl-CoA synthetase, fatty acyl activating enzyme, acyl-CoA oxidase, acyl-CoA thioesterase, hexanoyl-CoA synthetase, acetyl-CoA carboxylase, enoyl-CoA hydratase, 3-hydroxyacyl-CoA dehydrogenase, beta-ketothiolase, thiolase, acyl-CoA synthase, cannabidiolic acid synthase, tetrahydrocannabidiolic acid, olivetolic acid synthase, or polyketide synthase.

17. The genetically modified microorganism of claim 13, wherein the biosynthetic enzyme comprises a peroxisomal targeting sequence.

18. The genetically modified microorganism of claim 17, wherein the peroxisomal targeting sequence has a consensus sequence of [S/A/H/C/E/P/Q/V]-[K/R/H/Q]-[L/F] as set forth in SEQ ID NO: 7, or GRRAKL as set forth in SEQ ID NO: 6.

19. The genetically modified microorganism of claim 13, wherein the microorganism is capable of producing in the peroxisome a fatty acyl-CoA.

20. The genetically modified microorganism of claim 19, wherein the fatty acyl-CoA is acetyl-CoA, malonyl-CoA, or hexanoyl-CoA.

21. The genetically modified microorganism of claim 13, wherein the enzyme is removed of its endogenous amino-terminal localization sequence and/or carboxyl-terminal localization sequence.
